(19) United States
(12) Patent Application Publication
(10) Pub. No.: US 2022/0048899 A1
(43) Pub. Date: Feb. 17, 2022

(54) HETEROCYCLIC COMPOUND, ORGANIC LIGHT-EMITTING DEVICE COMPRISING SAME, METHOD FOR MANUFACTURING SAME, AND COMPOSITION FOR ORGANIC MATERIAL LAYER

(71) Applicant: LT MATERIALS CO., LTD., Yongin-Si, Gyeonggi-do (KR)

(72) Inventors: Su-Yeon KIM, Yongin-si (KR); Geon-Yu PARK, Yongin-si (KR); Dong-Jun KIM, Yongin-si (KR)

(73) Assignee: LT MATERIALS CO., LTD., Yongin-Si, Gyeonggi-do (KR)

(21) Appl. No.: 17/291,061

(22) PCT Filed: Dec. 11, 2019
(86) PCT No.: PCT/KR2019/017447

§ 371 Date: May 4, 2021

(30) Foreign Application Priority Data
Dec. 11, 2018 (KR) 10-2018-0158770

(51) Int. Cl. C07D 405/14; C07D 495/04; C07D 471/04; C07D 417/14; C07D 409/14; C07F 9/53; C07F 7/08; C07D 405/04; C07D 417/04; C07D 405/10; C07D 403/14; C07D 403/10; H01L 51/00

(52) U.S. Cl. CPC C07D 405/14; H01L 51/5012; C07D 471/04; C07D 417/14; C07D 409/14; C07F 9/5325; C07F 7/0812; C07D 405/04; C07D 417/04; C07D 405/10; C07D 403/14; C07D 403/10; H01L 51/0054; H01L 51/0056; H01L 51/0058; H01L 51/006; H01L 51/0061; H01L 51/0067; H01L 51/0072; H01L 51/0073; H01L 51/0074; H01L 51/0094; H01L 51/001; C07D 495/04

(57) ABSTRACT

The present specification relates to a heterocyclic compound represented by Chemical Formula 1, an organic light emitting device comprising the same, a method for manufacturing the same, and a composition for an organic material layer.

### TECHNICAL FIELD

This application claims priority to and the benefits of Korean Patent Application No. 10-2018-0158770, filed with the Korean Intellectual Property Office on Dec. 11, 2018, the entire contents of which are incorporated herein by reference.

The present specification relates to a heterocyclic compound, an organic light emitting device comprising the same, a method for manufacturing the same, and a composition for an organic material layer.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENT

### Patent Document

U.S. Pat. No. 4,356,429

### DISCLOSURE

### Technical Problem

The present disclosure is directed to providing a heterocyclic compound, an organic light emitting device comprising the same, a method for manufacturing the same, and a composition for an organic material layer.

### Technical Solution

One embodiment of the present application provides a heterocyclic compound represented by the following Chemical Formula 1.

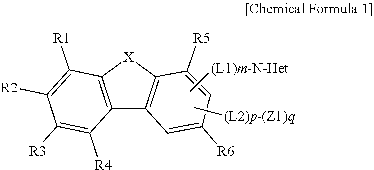

In Chemical Formula 1,

N-Het is a monocyclic or polycyclic heterocyclic group substituted or unsubstituted and comprising one or more Ns,

L1 and L2 are the same as or different from each other, and each independently a direct bond; a substituted or unsubstituted arylene group; or a substituted or unsubstituted heteroarylene group,

Z1 is selected from the group consisting of deuterium; halogen; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —P(═O)RR′; —SiRR′R″; and a substituted or unsubstituted amine group,

X is O; S; or NR7,

R7 is a substituted or unsubstituted alkyl group; a substituted or unsubstituted aryl group; or a substituted or unsubstituted heteroaryl group,

R1 to R6 are the same as or different from each other, and each independently selected from the group consisting of hydrogen; deuterium; halogen; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —P(═O)RR′; —SiRR′R″; and a substituted or unsubstituted amine group, or two or more groups adjacent to each other bond to each other to form a substituted or unsubstituted aliphatic or aromatic hydrocarbon ring or a substituted or unsubstituted heteroring,

R, R′ and R″ are the same as or different from each other, and each independently a substituted or unsubstituted alkyl group; a substituted or unsubstituted aryl group; or a substituted or unsubstituted heteroaryl group,

m and p are an integer of 0 to 3, and

q is an integer of 1 to 6.

In addition, one embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In addition, one embodiment of the present application provides an organic light emitting device, wherein the organic material layer comprising the heterocyclic compound of Chemical Formula 1 further comprises a heterocyclic compound represented by the following Chemical Formula 2.

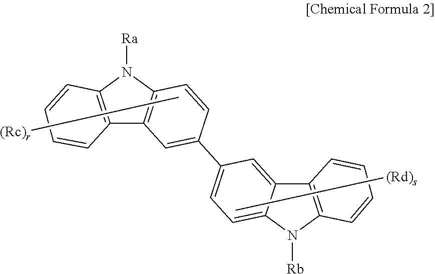

In Chemical Formula 2,

Rc and Rd are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiR₁₀R₁₁R₁₂; −P(═O)R₁₀R₁₁; and an amine group unsubstituted or substituted with a substituted or unsubstituted alkyl group, a substituted or unsubstituted aryl group or a substituted or unsubstituted heteroaryl group, or two or more groups adjacent to each other bond to each other to form a substituted or unsubstituted aromatic hydrocarbon ring or a substituted or unsubstituted heteroring,

R₁₀, R₁₁ and R₁₂ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group,

Ra and Rb are the same as or different from each other, and each independently a substituted or unsubstituted aryl group; or a substituted or unsubstituted heteroaryl group, and

r and s are an integer of 0 to 7.

In addition, another embodiment of the present application provides a composition for an organic material layer of an organic light emitting device, the composition comprising the heterocyclic compound represented by Chemical Formula 1 and the heterocyclic compound represented by Chemical Formula 2.

Lastly, one embodiment of the present application provides a method for manufacturing an organic light emitting device, the method comprising preparing a substrate; forming a first electrode on the substrate; forming one or more organic material layers on the first electrode; and forming a second electrode on the organic material layer, wherein the forming of organic material layers comprises forming one or more organic material layers using the composition for an organic material layer according to one embodiment of the present application.

### Advantageous Effects

A compound described in the present specification can be used as a material of an organic material layer of an organic light emitting device. The compound is capable of performing a role of a hole injection material, a hole transfer material, a light emitting material, an electron transfer material, an electron injection material and the like in an organic light emitting device. Particularly, the compound can be used as a light emitting layer material of an organic light emitting device.

Specifically, the compound can be used alone as a light emitting material, or can be used as a host material or a dopant material of a light emitting layer. When using the compound represented by Chemical Formula 1 in an organic material layer, a driving voltage of a device can be lowered, light efficiency can be enhanced, and lifetime properties of a device can be enhanced by thermal stability of the compound.

In addition, by having specific substituents at No. 1 and No. 3 positions of the core structure as in the heterocyclic compound represented by Chemical Formula 1, the HOMO orbital can be vertically delocalized decreasing hole mobility, and as a result, holes and electrons are evenly balanced in a light emitting layer, and an increased lifetime is obtained when used in a device.

Particularly, the heterocyclic compound represented by Chemical Formula 1 and the heterocyclic compound represented by Chemical Formula 2 can be used as a material of a light emitting layer of an organic light emitting device at the same time. In this case, a driving voltage of the device can be lowered, light efficiency can be enhanced, and lifetime properties of the device can be particularly enhanced by thermal stability of the compound.

### DESCRIPTION OF DRAWINGS

FIG. 1 to FIG. 3 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

In the present specification, a term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benxyloxy, p-methylbenzyloxy and the like, but are not limited thereto.

In the present specification, the cycloalkyl group comprises monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the phosphine oxide group is represented by —P(═O) R₁₀₁R₁₀₂, and R₁₀₁ and R₁₀₂ are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, examples described above may be used. Examples of the phosphine oxide group may comprise a diphenylphosphine oxide group, a dinaphthylphosphine oxide group and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

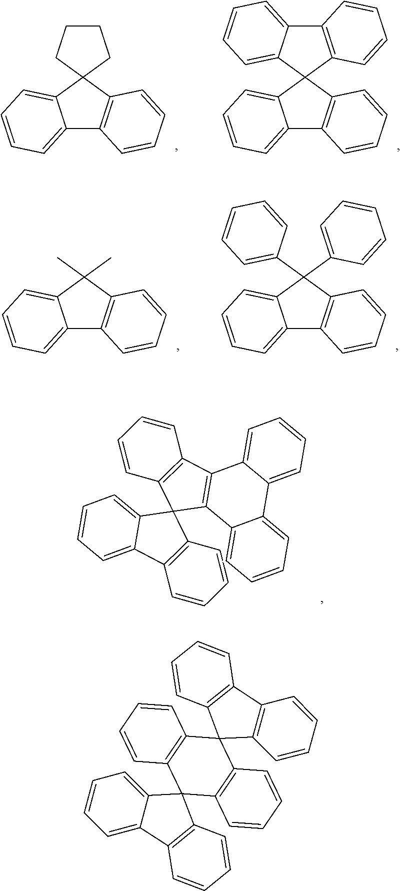

and the like may be included, however, the structure is not limited thereto.

In the present specification, the heteroaryl group comprises O, S, Se, N or Si as a heteroatom, comprises monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. Descriptions on the aryl group provided above may be applied thereto except for those that are each a divalent. In addition, the heteroarylene group means the heteroaryl group having two bonding sites, that is, a divalent group. Descriptions on the heteroaryl group provided above may be applied thereto except for those that are each a divalent.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

One embodiment of the present application provides a compound represented by Chemical Formula 1.

In one embodiment of the present application, Chemical Formula 1 may be represented by the following Chemical Formula 3 or Chemical Formula 4.

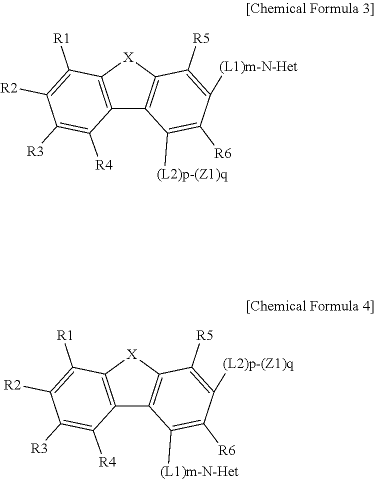

In Chemical Formulae 3 and 4,

R1 to R6, L1, L2, Z1, N-Het, X, m, p and q have the same definitions as in Chemical Formula 1.

In one embodiment of the present application, R1 to R6 are the same as or different from each other, and each independently selected from the group consisting of hydrogen; deuterium; halogen; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —P(═O)RR′; —SiRR′R″; and a substituted or unsubstituted amine group, or two or more groups adjacent to each other may bond to each other to form a substituted or unsubstituted aliphatic or aromatic hydrocarbon ring or a substituted or unsubstituted heteroring.

In another embodiment, R1 to R6 are the same as or different from each other, and may be each independently selected from the group consisting of hydrogen; a substituted or unsubstituted alkyl group; a substituted or unsubstituted aryl group; a substituted or unsubstituted heteroaryl group; and a substituted or unsubstituted amine group.

In another embodiment, R1 to R6 are the same as or different from each other, and may be each independently selected from the group consisting of hydrogen; a substituted or unsubstituted C1 to C60 alkyl group; a substituted or unsubstituted C6 to C60 aryl group; a substituted or unsubstituted C2 to C60 heteroaryl group; and a substituted or unsubstituted amine group.

In another embodiment, R1 to R6 are the same as or different from each other, and may be each independently selected from the group consisting of hydrogen; a substituted or unsubstituted C1 to C40 alkyl group; a substituted or unsubstituted C6 to C40 aryl group; a substituted or unsubstituted C2 to C40 heteroaryl group; and a substituted or unsubstituted amine group.

In another embodiment, R1 to R6 may be hydrogen.

In one embodiment of the present application, X may be O.

In one embodiment of the present application, X may be S.

In one embodiment of the present application, X may be NR7.

Particularly, when X is O or S, an excellent electron transfer ability is obtained by having O and S atoms having high electronegativity in the center of the core structure, and suitable properties are also obtained in exciton blocking.

In one embodiment of the present application, R7 may be a substituted or unsubstituted alkyl group; a substituted or unsubstituted aryl group; or a substituted or unsubstituted heteroaryl group.

In another embodiment, R7 may be a substituted or unsubstituted aryl group.

In another embodiment, R7 may be a substituted or unsubstituted C6 to C60 aryl group.

In another embodiment, R7 may be a substituted or unsubstituted C6 to C40 aryl group.

In another embodiment, R7 may be a C6 to C40 monocyclic or polycyclic aryl group.

In another embodiment, R7 may be a C6 to C40 monocyclic aryl group.

In another embodiment, R7 may be a phenyl group.

In one embodiment of the present application, the N-Het may be a monocyclic or polycyclic heterocyclic group substituted or unsubstituted and comprising one or more Ns.

In another embodiment, the N-Het may be a monocyclic or polycyclic heterocyclic group substituted or unsubstituted and comprising one or more and three or less Ns.

In another embodiment, the N-Het may be a monocyclic or polycyclic heterocyclic group substituted or unsubstituted and comprising one or more and two or less Ns.

In another embodiment, the N-Het may be a monocyclic or polycyclic C2 to C60 heterocyclic group unsubstituted or substituted with one or more substituents selected from the group consisting of a C6 to C60 aryl group and a C2 to C60 heteroaryl group, and comprising one or more Ns.

In another embodiment, the N-Het may be a triazine group; a pyrimidine group; a pyridine group; a quinoline group; a quinazoline group; a phenanthroline group; an imidazole group; a benzothiazole group; or a benzo[4,5]thieno[2,3-d]pyrimidine group unsubstituted or substituted with one or more substituents selected from the group consisting of a C6 to C60 aryl group and a C2 to C60 heteroaryl group.

In another embodiment, the N-Het may be a triazine group unsubstituted or substituted with one or more substituents selected from the group consisting of a phenyl group, a biphenyl group, a naphthyl group, a triphenylenyl group, a dibenzofuran group, a dibenzothiophene group, a pyridine group, a dimethylfluorene group, a diphenylfluorene group and a spirobifluorene group; a pyrimidine group unsubstituted or substituted with a phenyl group; a pyridine group unsubstituted or substituted with a phenyl group; a quinoline group unsubstituted or substituted with a phenyl group; a quinazoline group unsubstituted or substituted with a phenyl group; a phenanthroline group; an imidazole group unsubstituted or substituted with a phenyl group; a benzothiazole group; or a benzo[4,5]thieno[2,3-d]pyrimidine group unsubstituted or substituted with a phenyl group.

In one embodiment of the present application, the N-Het may be substituted again with —CN; a phenyl group; P(═O)RR′; or SiRR′R″.

In one embodiment of the present application, L1 and L2 are the same as or different from each other, and may be each independently a direct bond; a substituted or unsubstituted arylene group; or a substituted or unsubstituted heteroarylene group.

In another embodiment, L1 and L2 are the same as or different from each other, and may be each independently a direct bond; a substituted or unsubstituted C6 to C60 arylene group; or a substituted or unsubstituted C2 to C60 heteroarylene group.

In another embodiment, L1 and L2 are the same as or different from each other, and may be each independently a direct bond; a substituted or unsubstituted C6 to C40 arylene group; or a substituted or unsubstituted C2 to C40 heteroarylene group.

In another embodiment, L1 and L2 are the same as or different from each other, and may be each independently a direct bond; a C6 to C40 arylene group; or a C2 to C40 heteroarylene group.

In another embodiment, L1 and L2 are the same as or different from each other, and may be each independently a direct bond; a C6 to C40 arylene group; or a C2 to C40 heteroarylene group comprising N as a heteroatom.

In another embodiment, L1 and L2 are the same as or different from each other, and may be each independently a direct bond; a phenylene group; a biphenylene group; a naphthylene group; or a divalent pyridine group.

In one embodiment of the present application, Z1 may be selected from the group consisting of deuterium; halogen; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —P(═O)RR′; —SiRR′R″; and a substituted or unsubstituted amine group.

In one embodiment of the present application, Z1 is —CN; or a substituted or unsubstituted amine group, or may be represented by the following Chemical Formula 1-1.

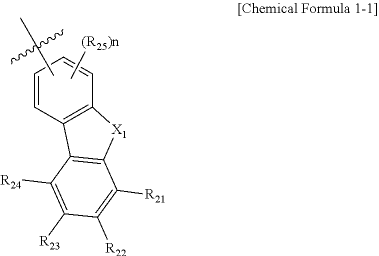

In Chemical Formula 1-1,

[symbol] means a site linked to L2 of Chemical Formula 1,

X₁ is O; S; NR₃₁; or CR₃₂R₃₃,

R₂₁ to R₂₅ are the same as or different from each other, and each independently selected from the group consisting of hydrogen; deuterium; halogen; —CN; a substituted or unsubstituted alkyl group; a substituted or unsubstituted aryl group; and a substituted or unsubstituted heteroaryl group, or two or more groups adjacent to each other bond to each other to form a substituted or unsubstituted aromatic ring, n is an integer of 0 to 3, and

R₃₁ to R₃₃ are the same as or different from each other, and each independently selected from the group consisting of a substituted or unsubstituted alkyl group; a substituted or unsubstituted aryl group; and a substituted or unsubstituted heteroaryl group, or two or more groups adjacent to each other may bond to each other to form a substituted or unsubstituted aromatic ring.

In one embodiment of the present application, Z1 is —CN; or an amine group unsubstituted or substituted with one or more substituents selected from the group consisting of a C6 to C60 aryl group and a C2 to C60 heteroaryl group, or may be represented by Chemical Formula 1-1.

In another embodiment, Z1 is —CN; or an amine group unsubstituted or substituted with one or more substituents selected from the group consisting of a C6 to C40 aryl group and a C2 to C40 heteroaryl group, or may be represented by Chemical Formula 1-1.

In another embodiment, Z1 is —CN; or an amine group unsubstituted or substituted with one or more substituents selected from the group consisting of a phenyl group, a biphenyl group, a naphthyl group, a dimethylfluorenyl group, a dibenzothiophene group and a dibenzofuran group, or may be represented by Chemical Formula 1-1.

In one embodiment of the present application, X1 may be O.

In one embodiment of the present application, X1 may be S.

In one embodiment of the present application, X1 may be NR₃₁.

In one embodiment of the present application, X1 may be CR₃₂R₃₃.

In one embodiment of the present application, R₃₁ to R₃₃ are the same as or different from each other, and each independently selected from the group consisting of a substituted or unsubstituted C1 to C60 alkyl group; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic ring.

In another embodiment, R₃₁ to R₃₃ are the same as or different from each other, and each independently selected from the group consisting of a substituted or unsubstituted C1 to C40 alkyl group; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic ring.

In another embodiment, R₃₁ to R₃₃ are the same as or different from each other, and each independently selected from the group consisting of a C1 to C40 alkyl group; and a C6 to C40 aryl group, or two or more groups adjacent to each other may bond to each other to form a C6 to C40 aromatic ring.

In another embodiment, R₃₁ to R₃₃ are the same as or different from each other, and each independently selected from the group consisting of a methyl group and a phenyl group, or two or more groups adjacent to each other may bond to each other to form a fluorenyl group.

In another embodiment, R₃₁ may be a phenyl group.

In another embodiment, R₃₂ and R₃₃ may be a methyl group.

In another embodiment, R₃₂ and R₃₃ may bond to each other to form a fluorenyl group.

In one embodiment of the present application, R₂₁ to R₂₅ are the same as or different from each other, and each independently selected from the group consisting of hydrogen; deuterium; halogen; —CN; a substituted or unsubstituted alkyl group; a substituted or unsubstituted aryl group; and a substituted or unsubstituted heteroaryl group, or two or more groups adjacent to each other may bond to each other to form a substituted or unsubstituted aromatic ring.

In another embodiment, R₂₁ to R₂₅ are the same as or different from each other, and each independently selected from the group consisting of a substituted or unsubstituted aryl group; and a substituted or unsubstituted heteroaryl group, or two or more groups adjacent to each other may bond to each other to form a substituted or unsubstituted aromatic ring.

In another embodiment, R₂₁ to R₂₅ are the same as or different from each other, and each independently selected from the group consisting of hydrogen; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic ring.

In another embodiment, R₂₁ to R₂₅ are the same as or different from each other, and each independently selected from the group consisting of hydrogen; a C6 to C60 aryl group; and a C2 to C60 heteroaryl group, or two or more groups adjacent to each other may bond to each other to form a C6 to C60 aromatic ring.

In another embodiment, R₂₁ to R₂₅ are the same as or different from each other, and each independently selected from the group consisting of hydrogen; a C6 to C40 aryl group; and a C2 to C40 heteroaryl group, or two or more groups adjacent to each other may bond to each other to form a C6 to C40 aromatic ring.

In another embodiment, R₂₁ to R₂₅ are the same as or different from each other, and each independently hydrogen; a phenyl group; a biphenyl group; a triphenylenyl group; or a dibenzothiophene group, or groups adjacent to each other may bond to each other to form a benzene ring.

In one embodiment of the present application, R, R′ and R″ are the same as or different from each other, and may be each independently a substituted or unsubstituted alkyl group; a substituted or unsubstituted aryl group; or a substituted or unsubstituted heteroaryl group.

In another embodiment, R, R′ and R″ are the same as or different from each other, and may be each independently a substituted or unsubstituted alkyl group; or a substituted or unsubstituted aryl group.

In another embodiment, R, R′ and R″ are the same as or different from each other, and may be each independently a substituted or unsubstituted C1 to C60 alkyl group; or a substituted or unsubstituted C6 to C60 aryl group.

In another embodiment, R, R′ and R″ are the same as or different from each other, and may be each independently a C1 to C60 alkyl group; or a C6 to C60 aryl group.

In another embodiment, R, R′ and R″ are the same as or different from each other, and may be each independently a methyl group; or a phenyl group.

In another embodiment, R, R′ and R″ may be a phenyl group.

In the heterocyclic compound provided in one embodiment of the present application, Chemical Formula 1 is represented by any one of the following compounds.

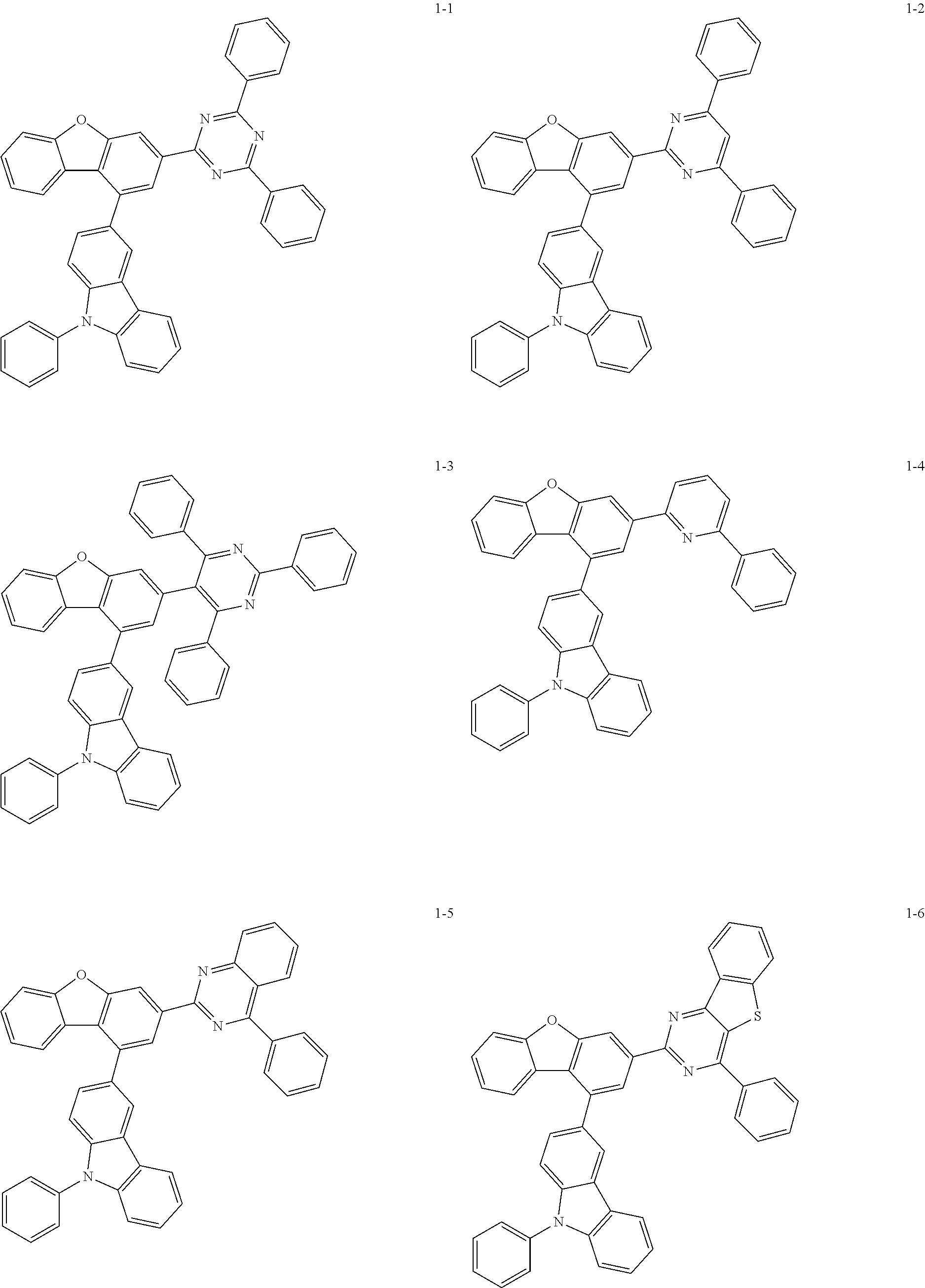

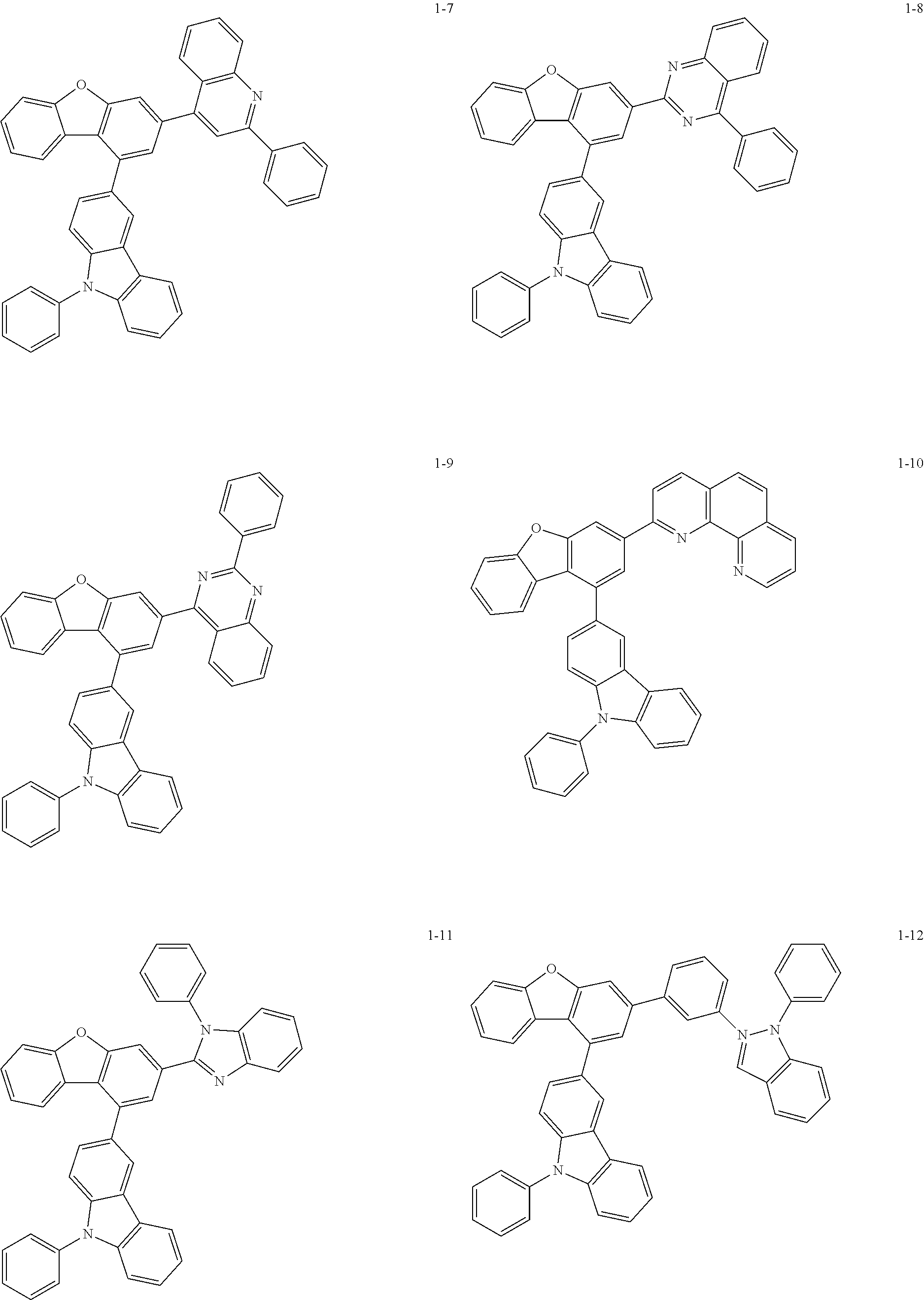

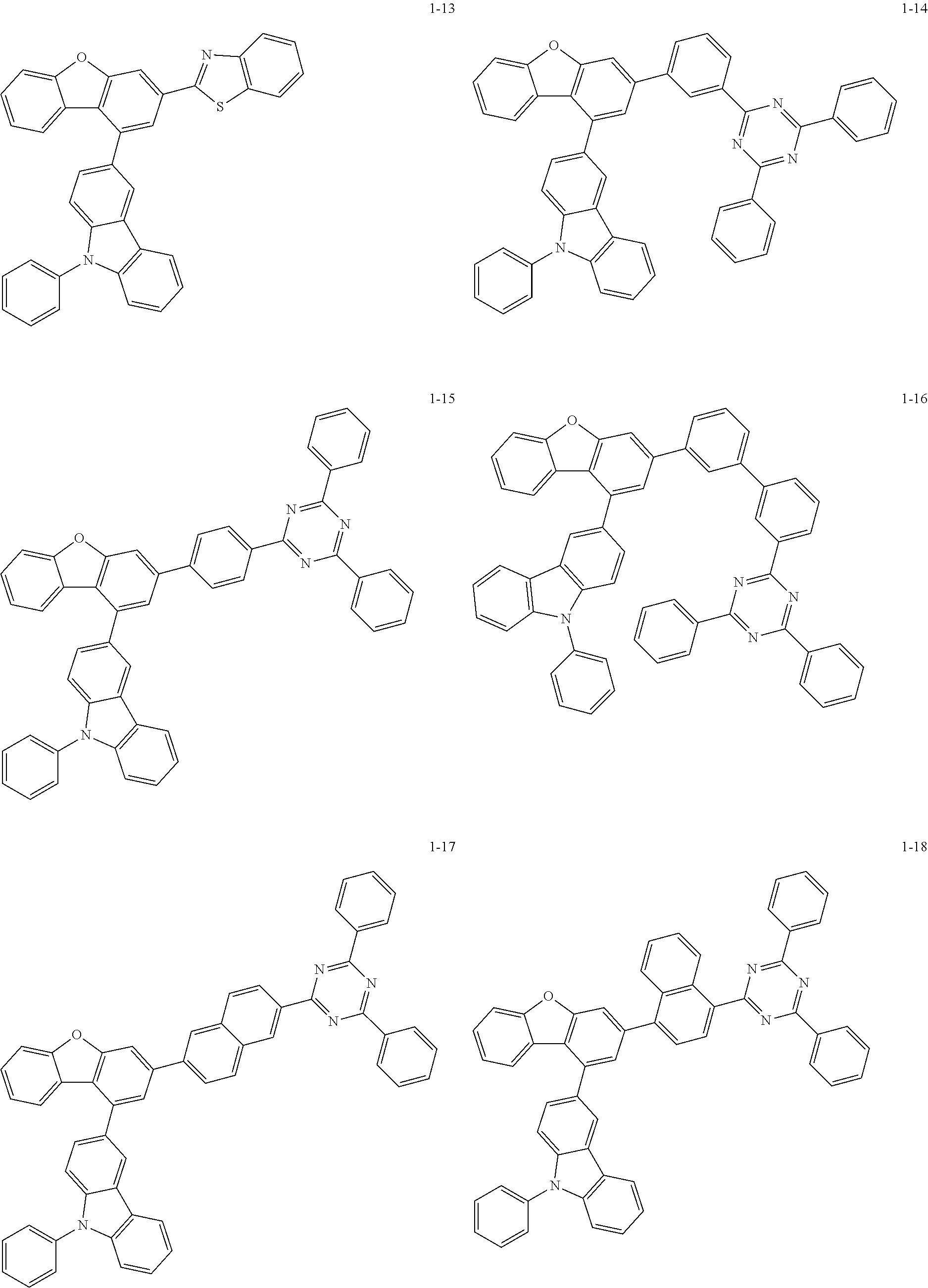

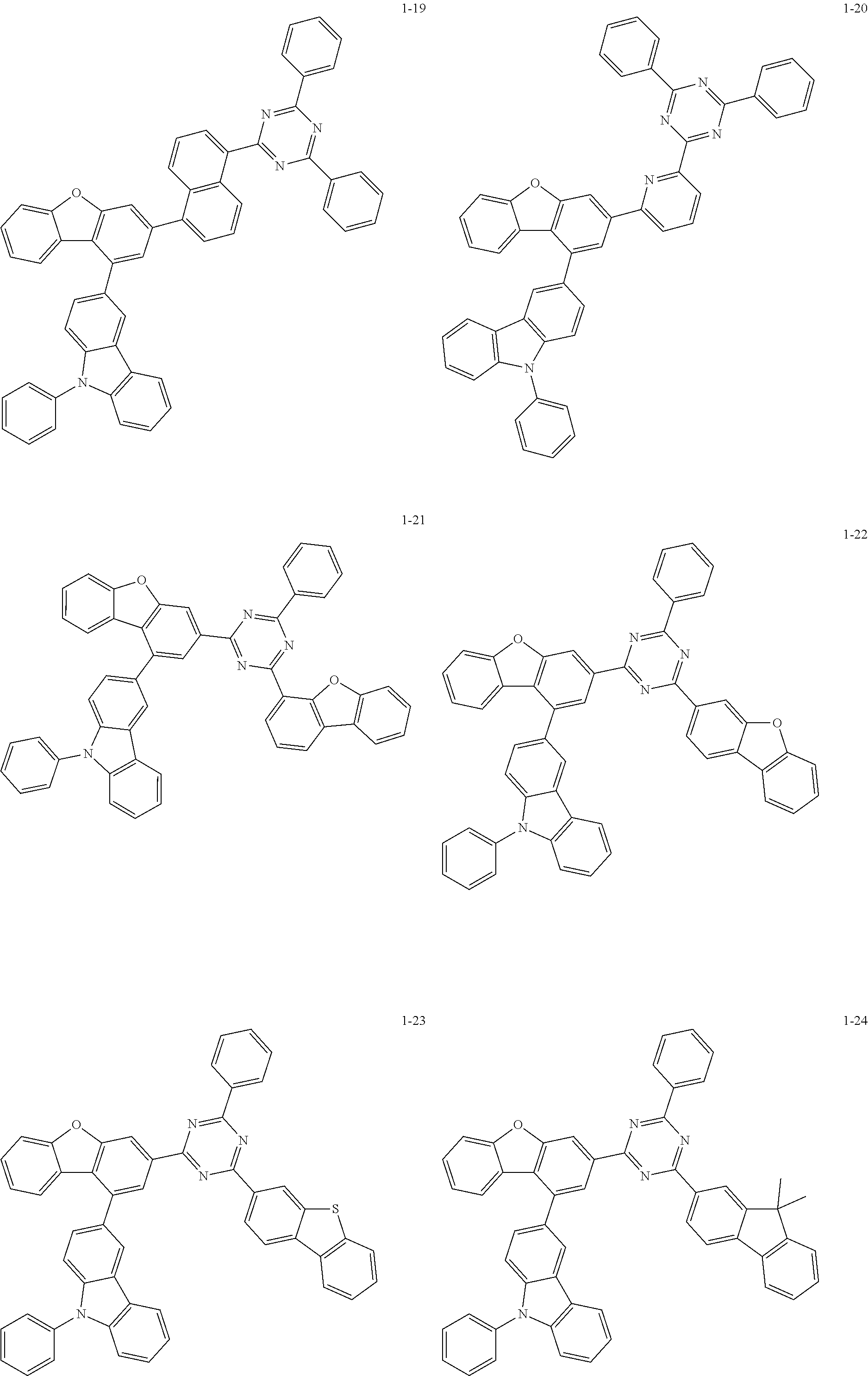

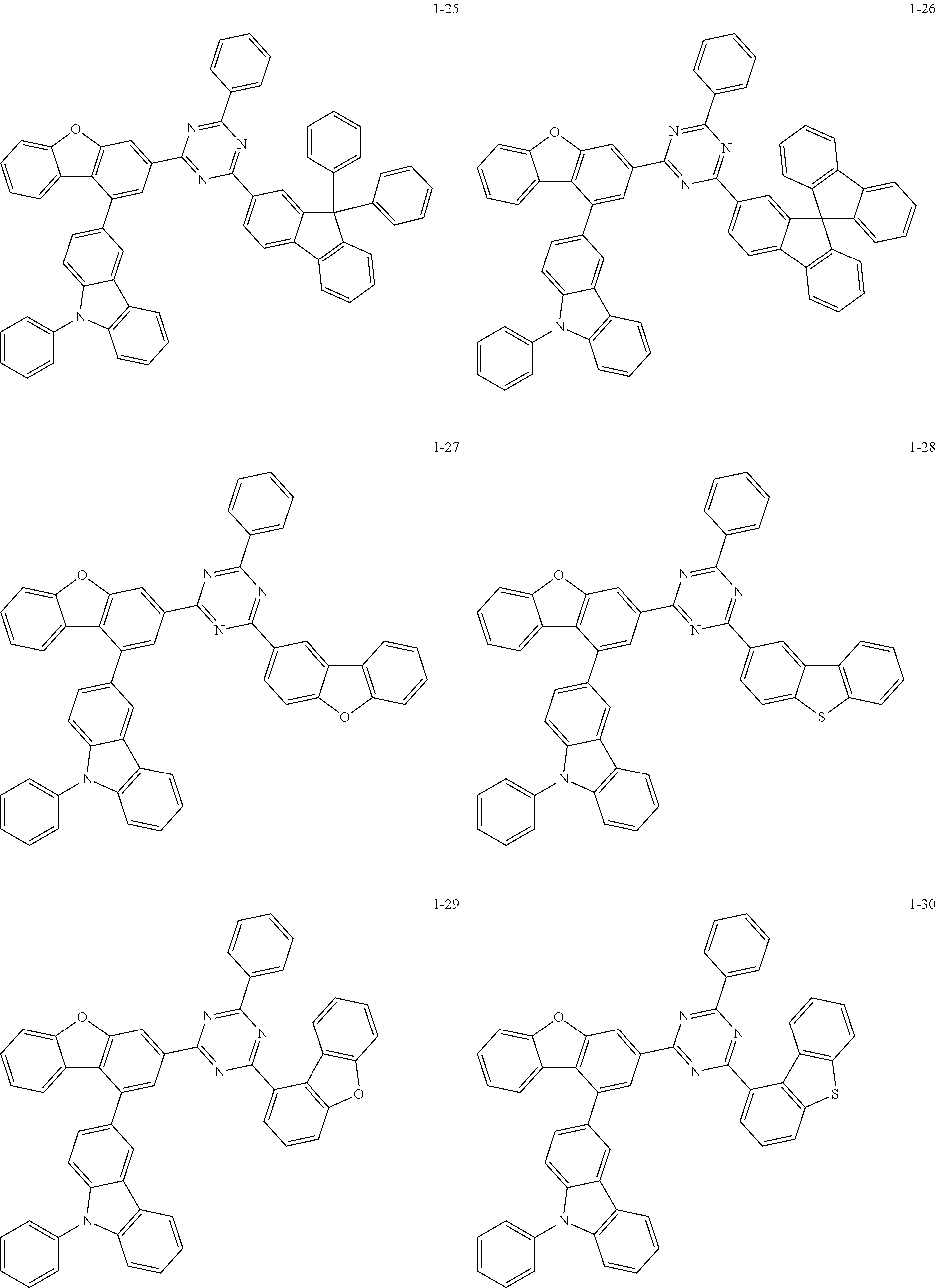

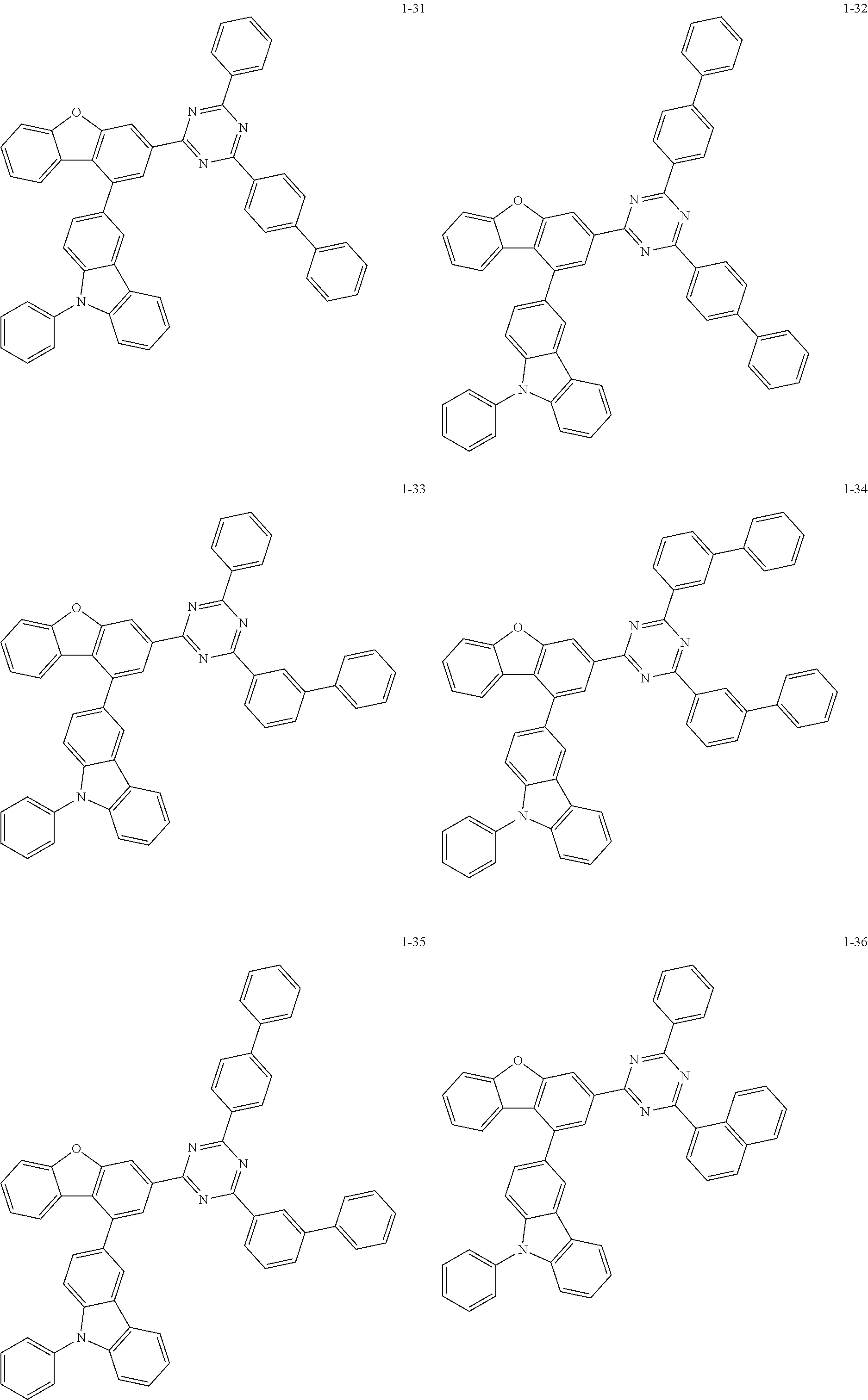

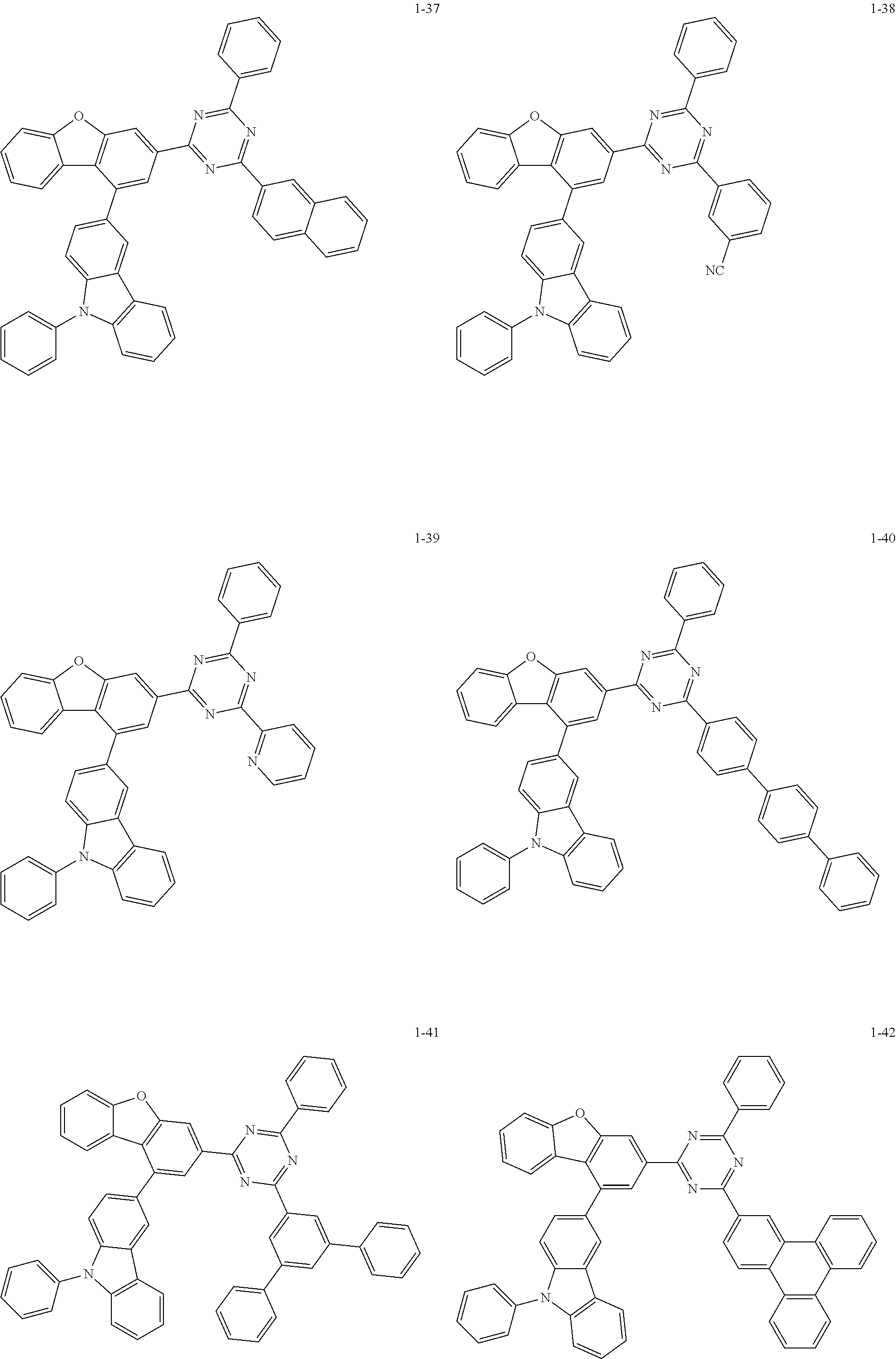

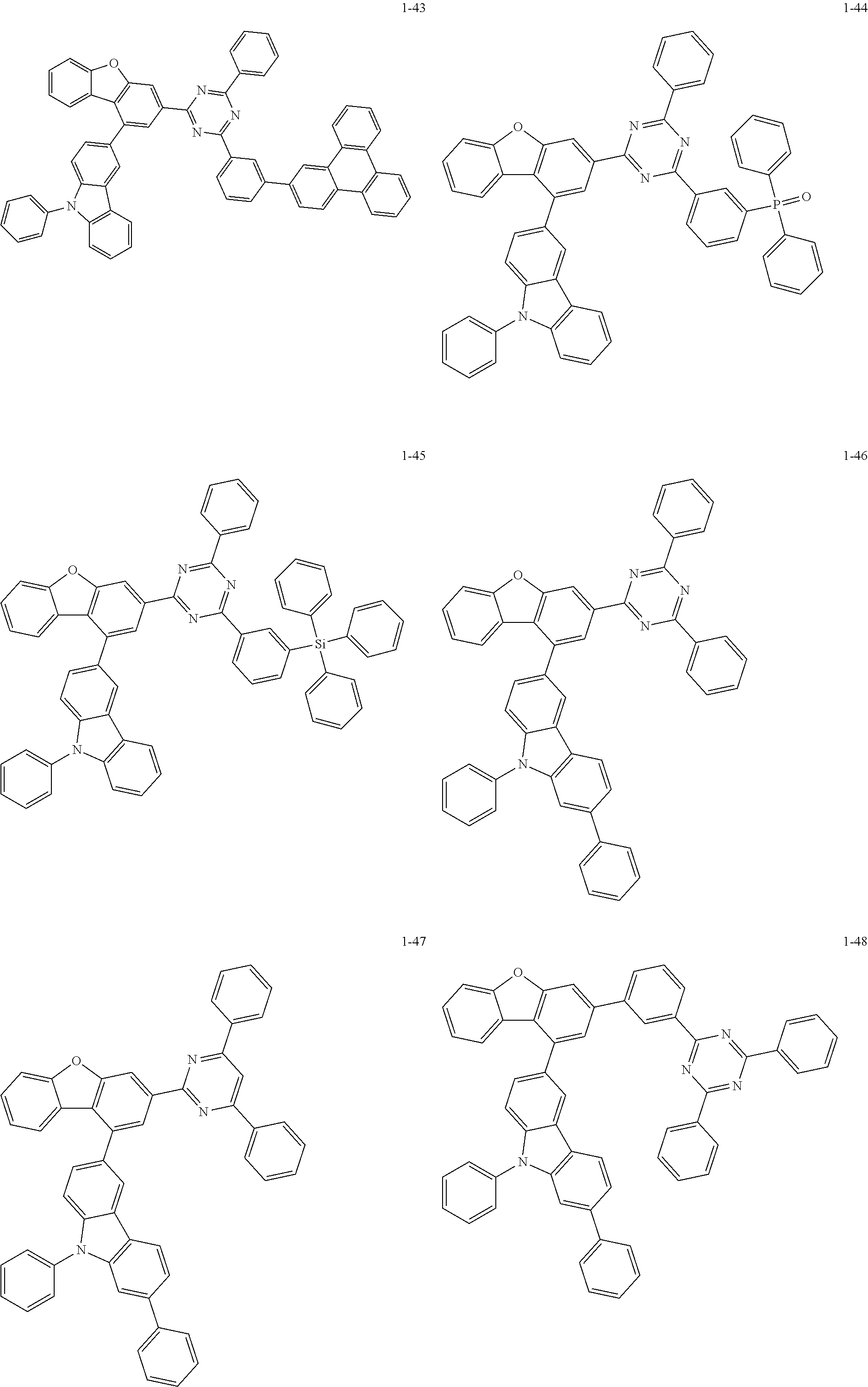

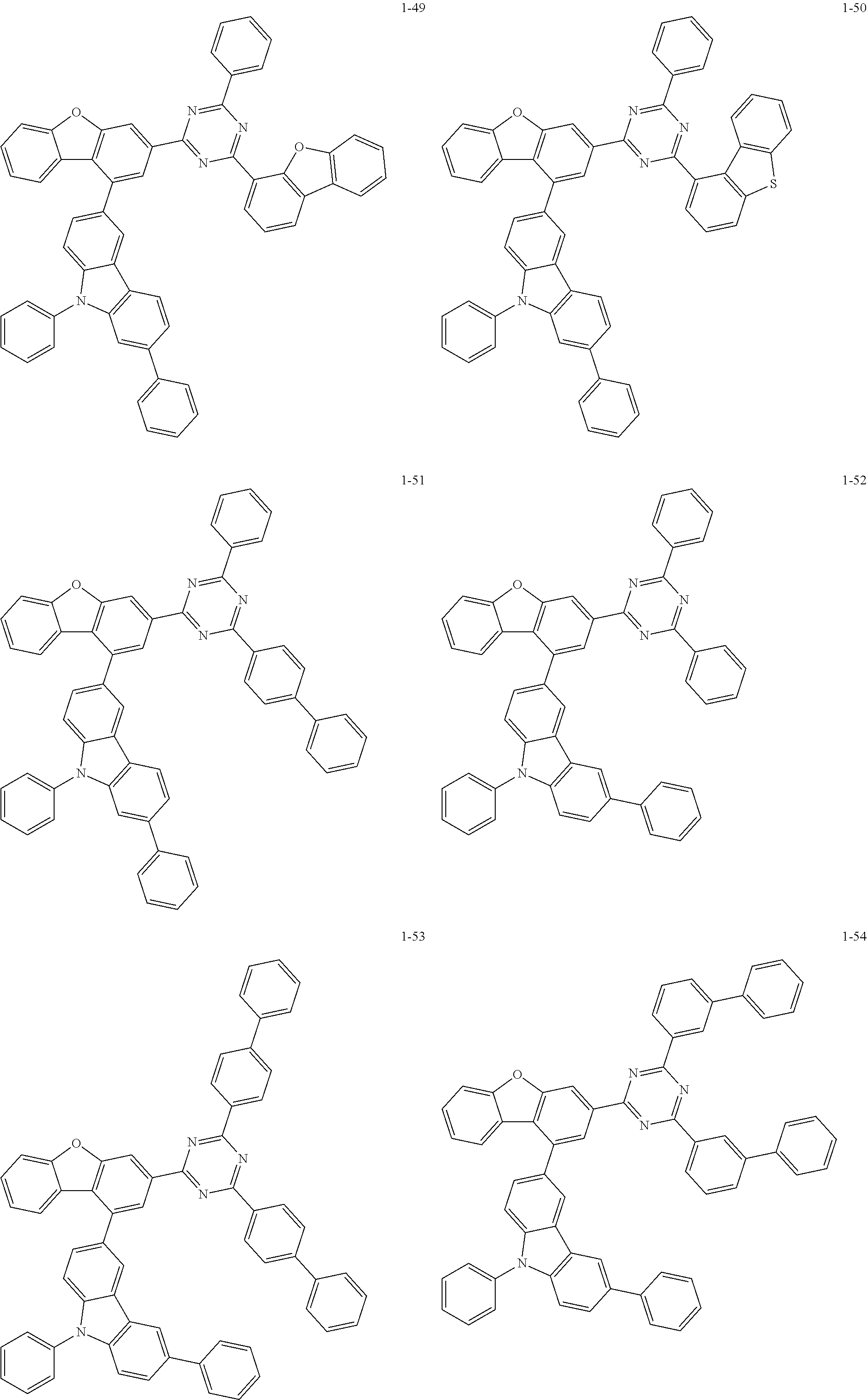

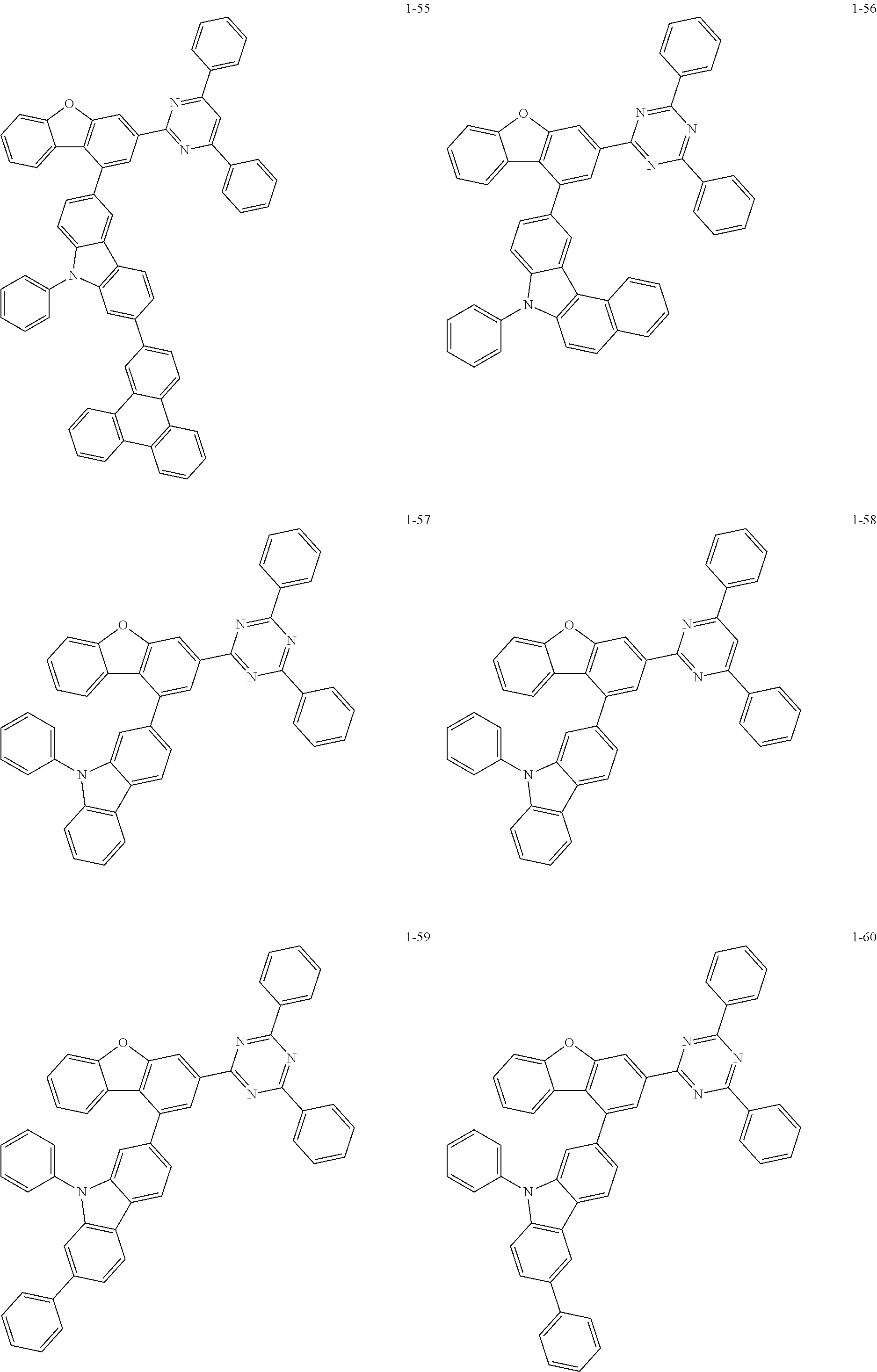

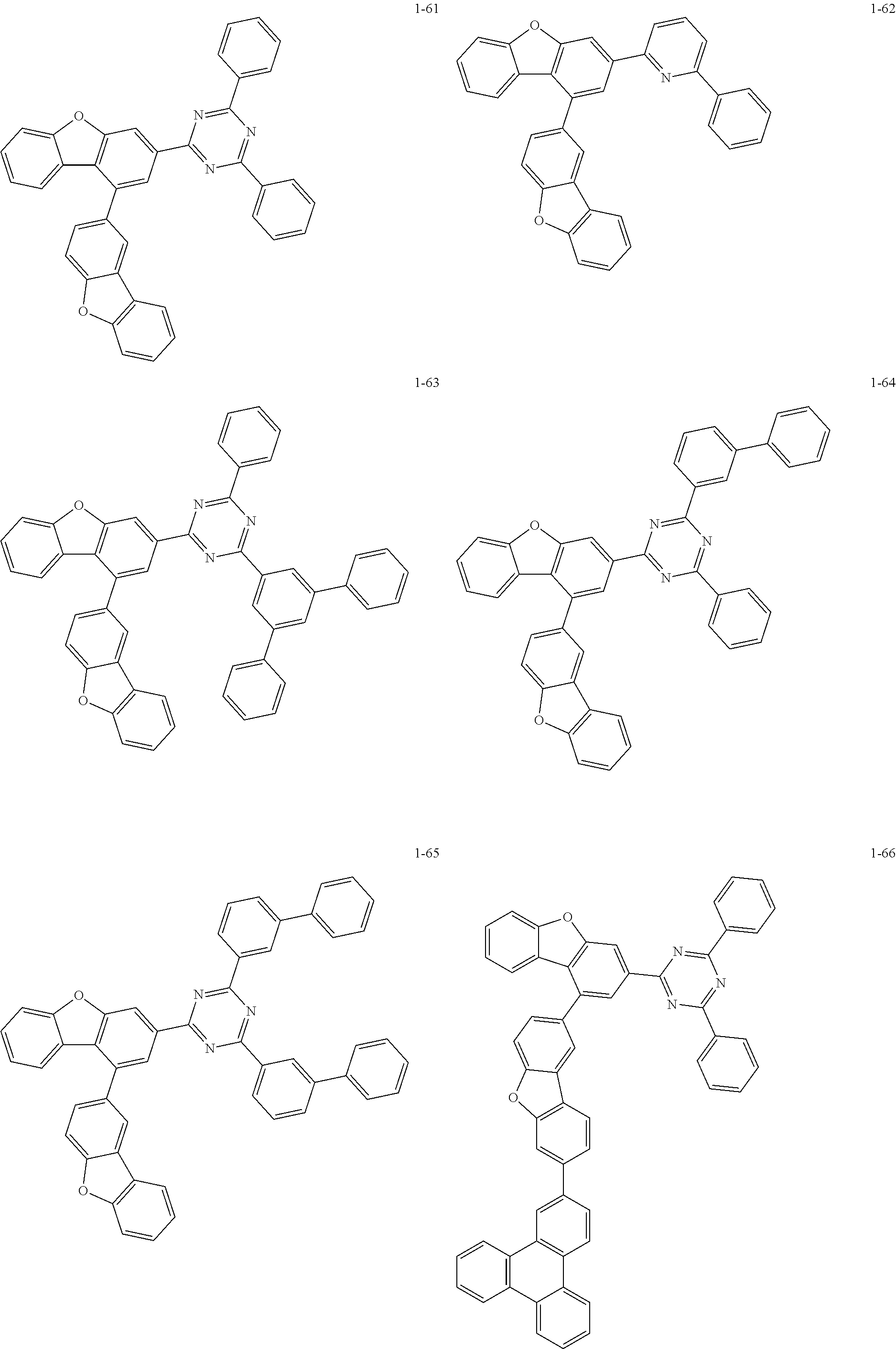

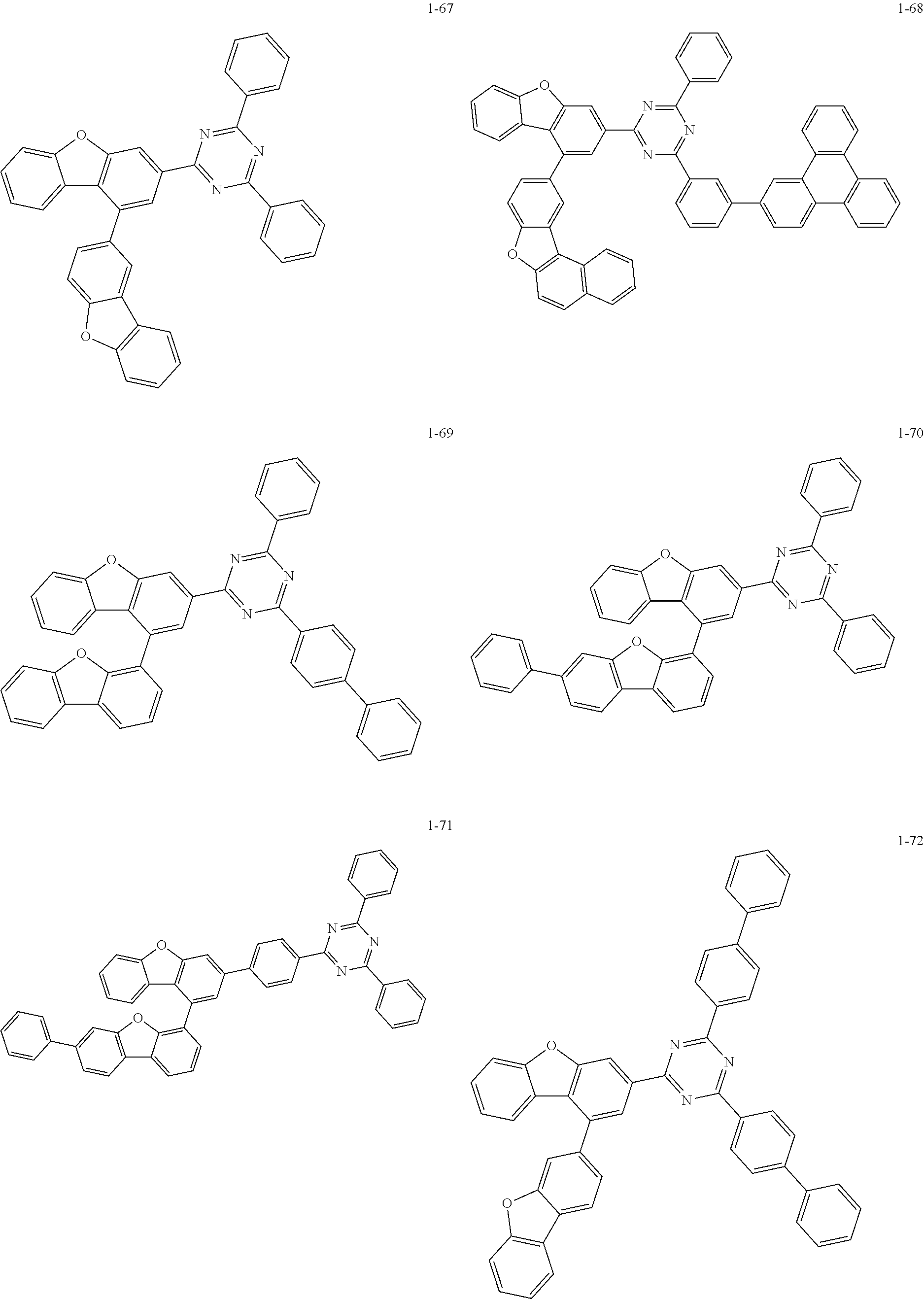

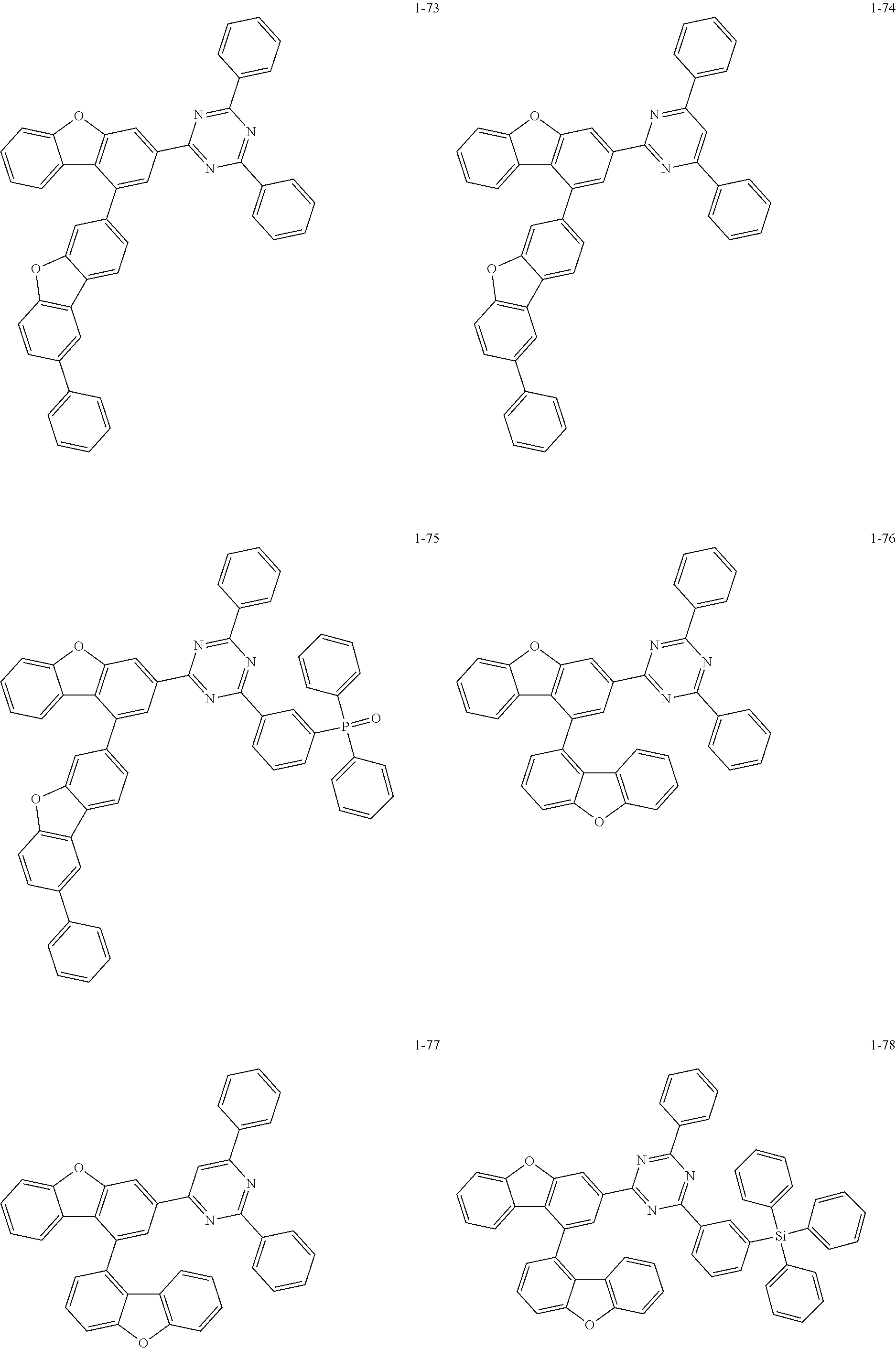

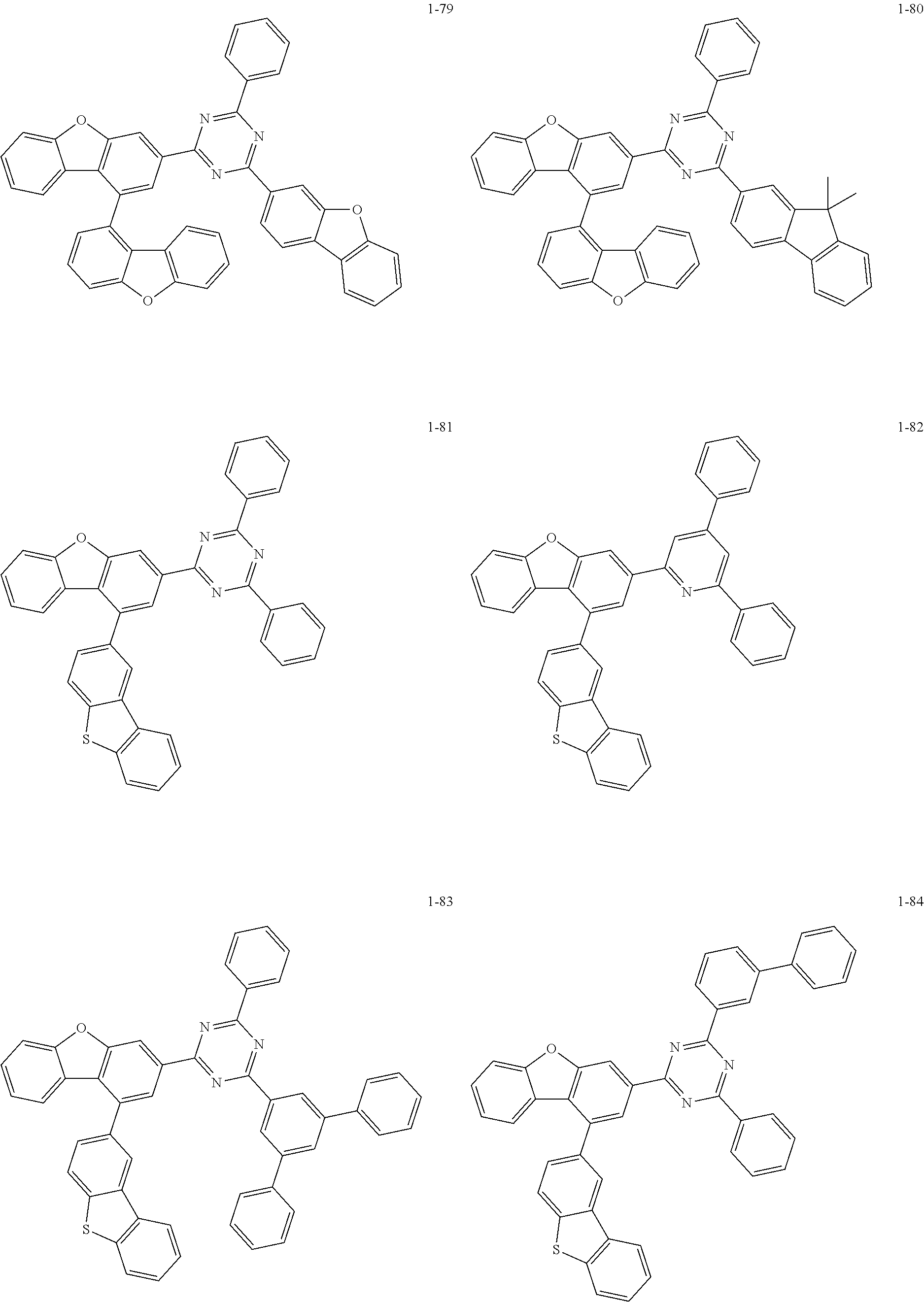

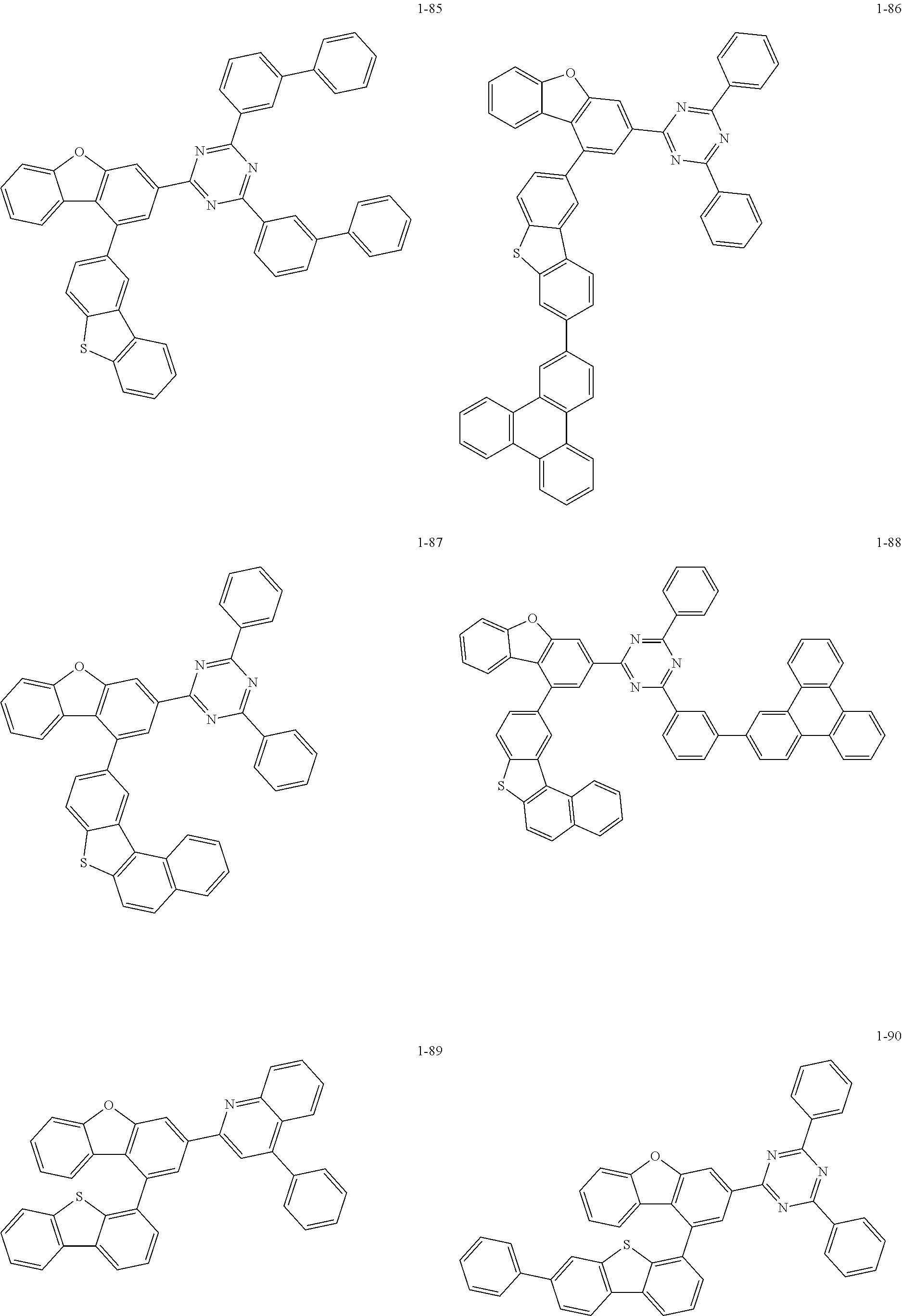

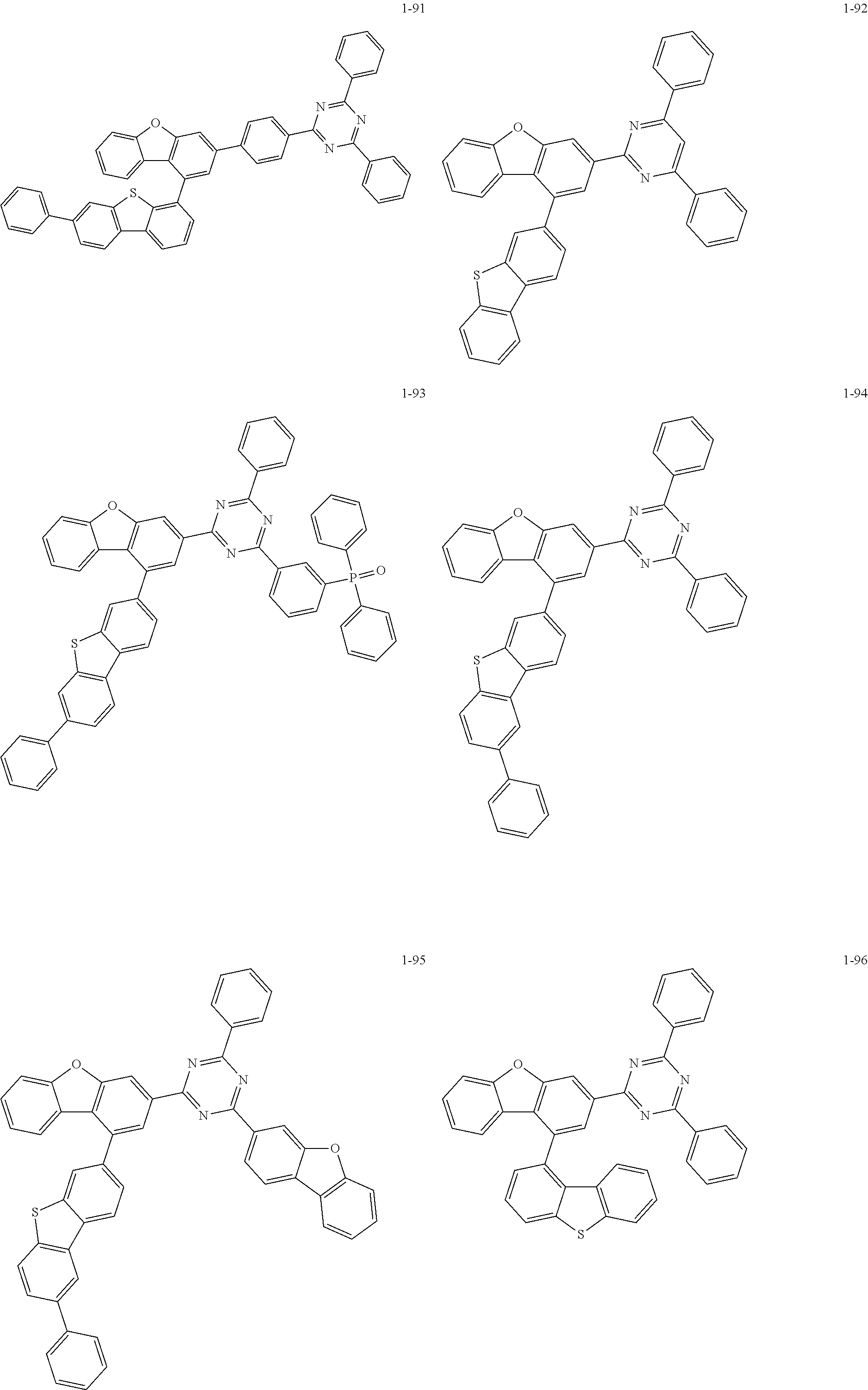

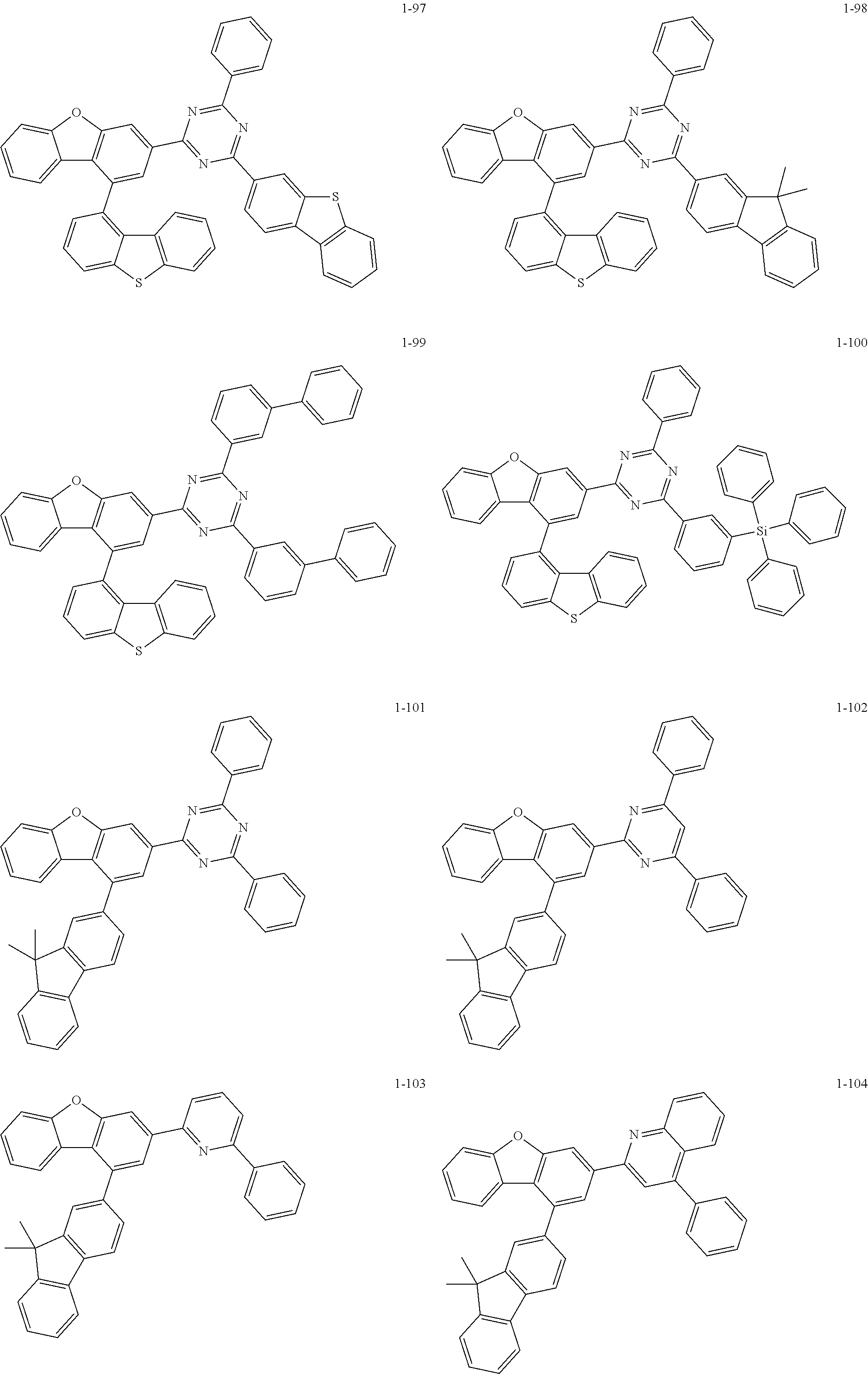

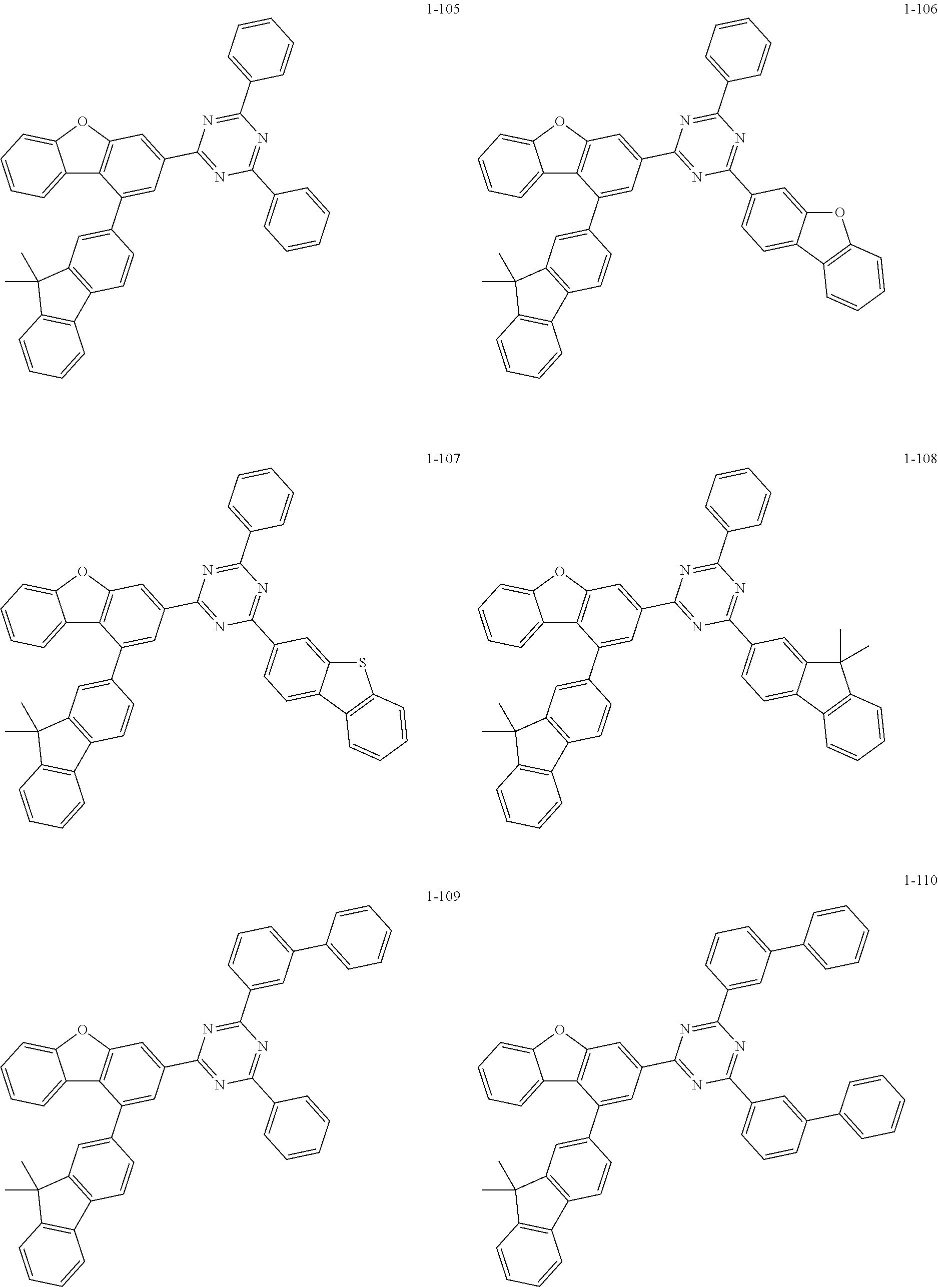

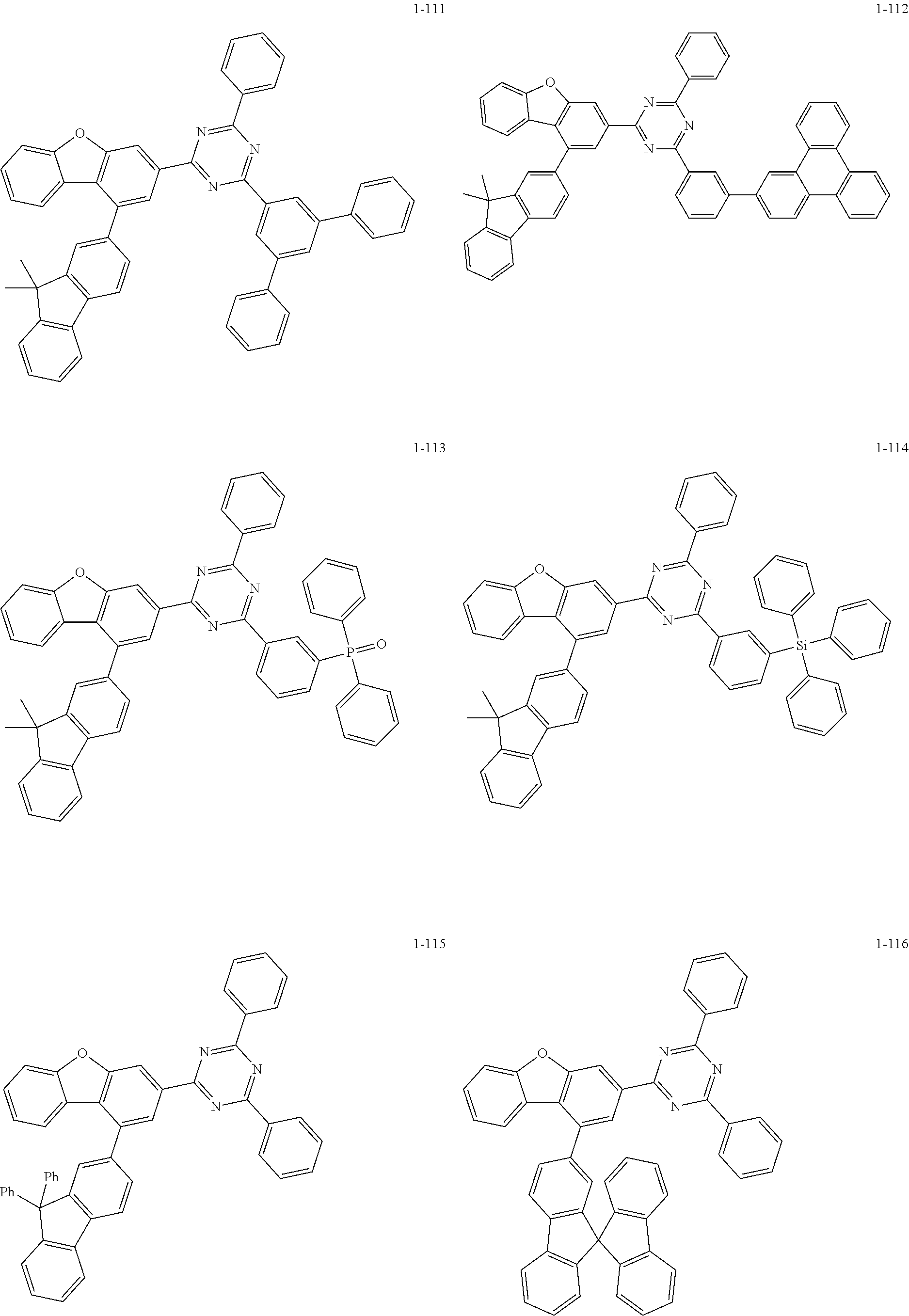

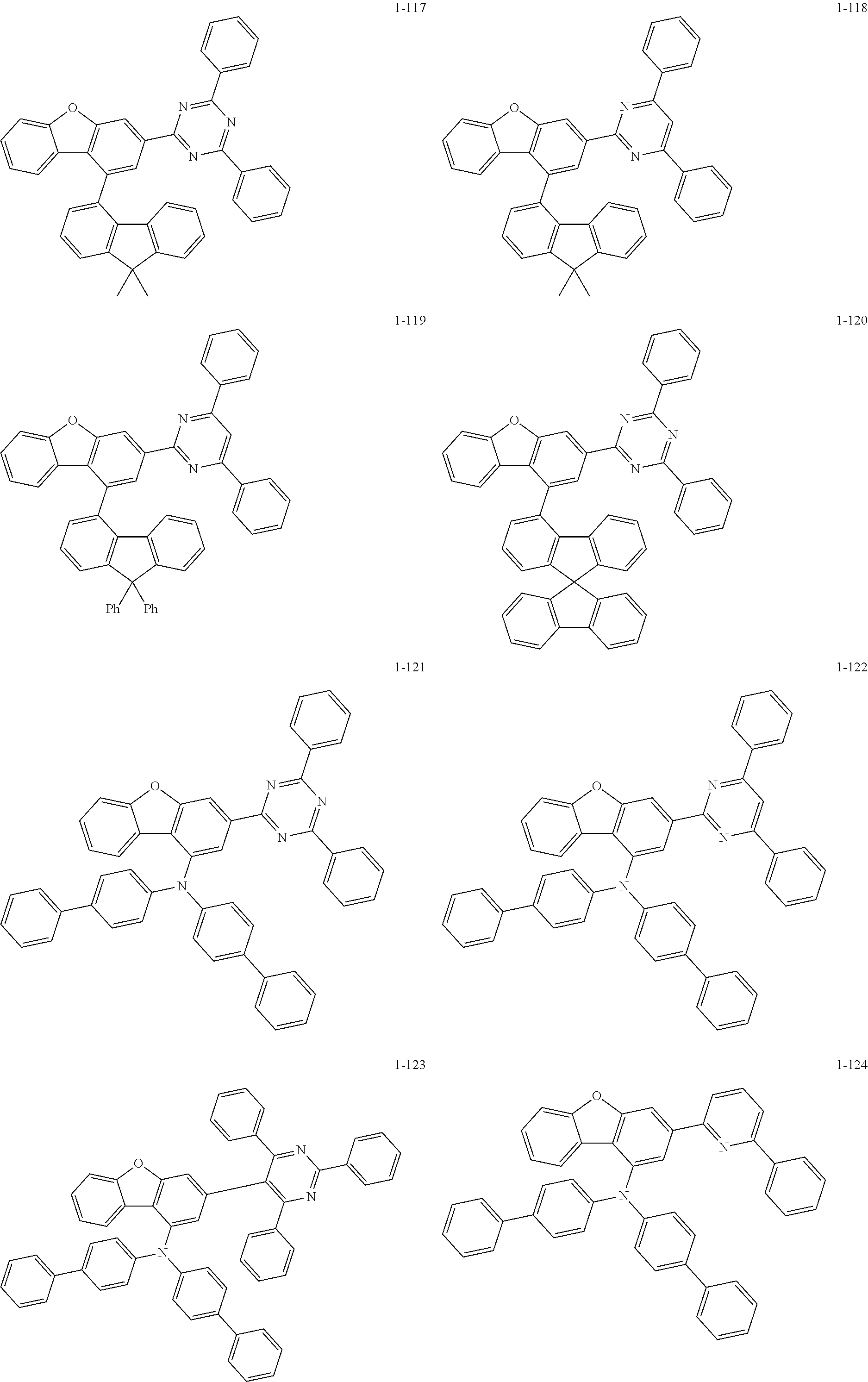

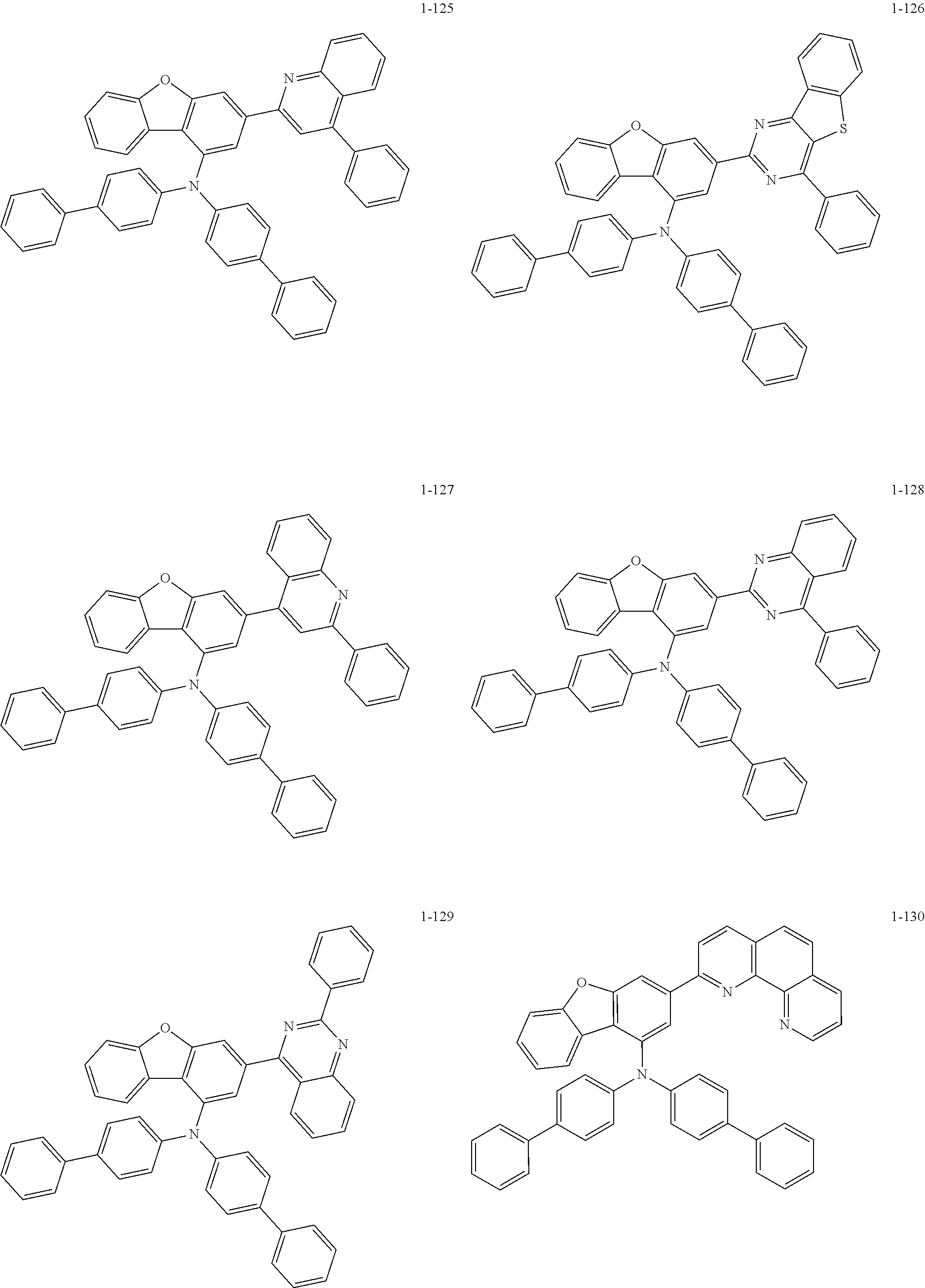

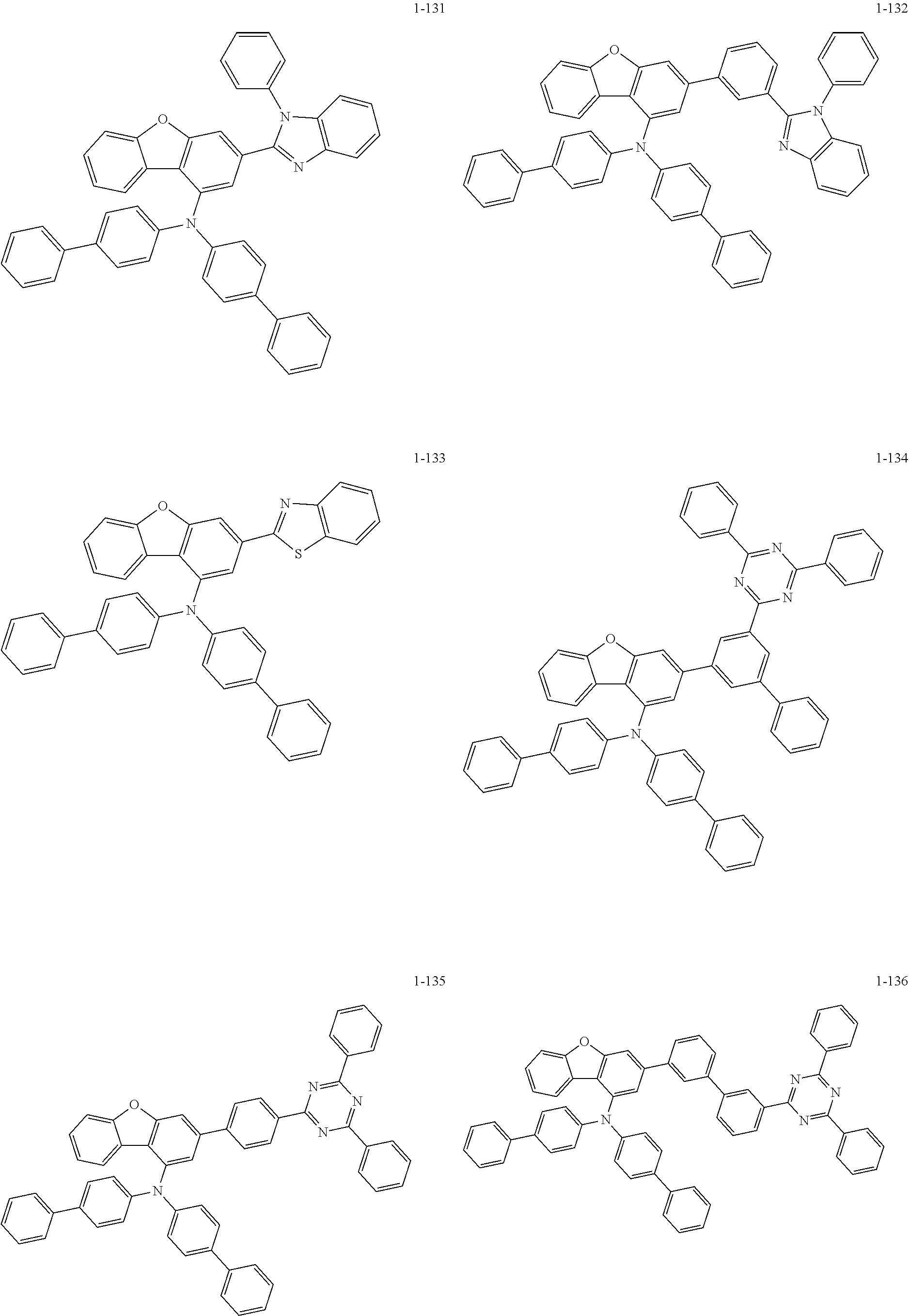

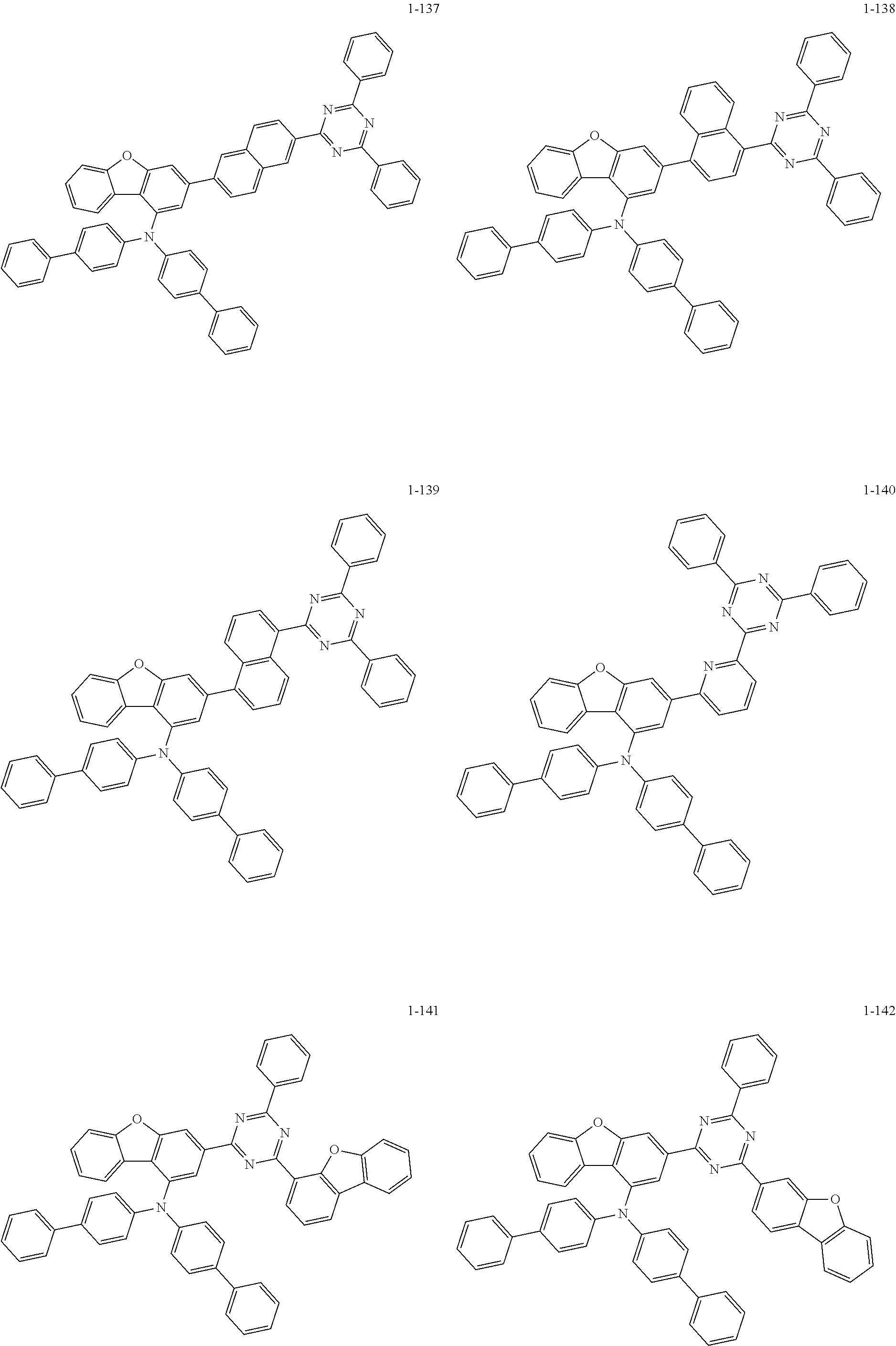

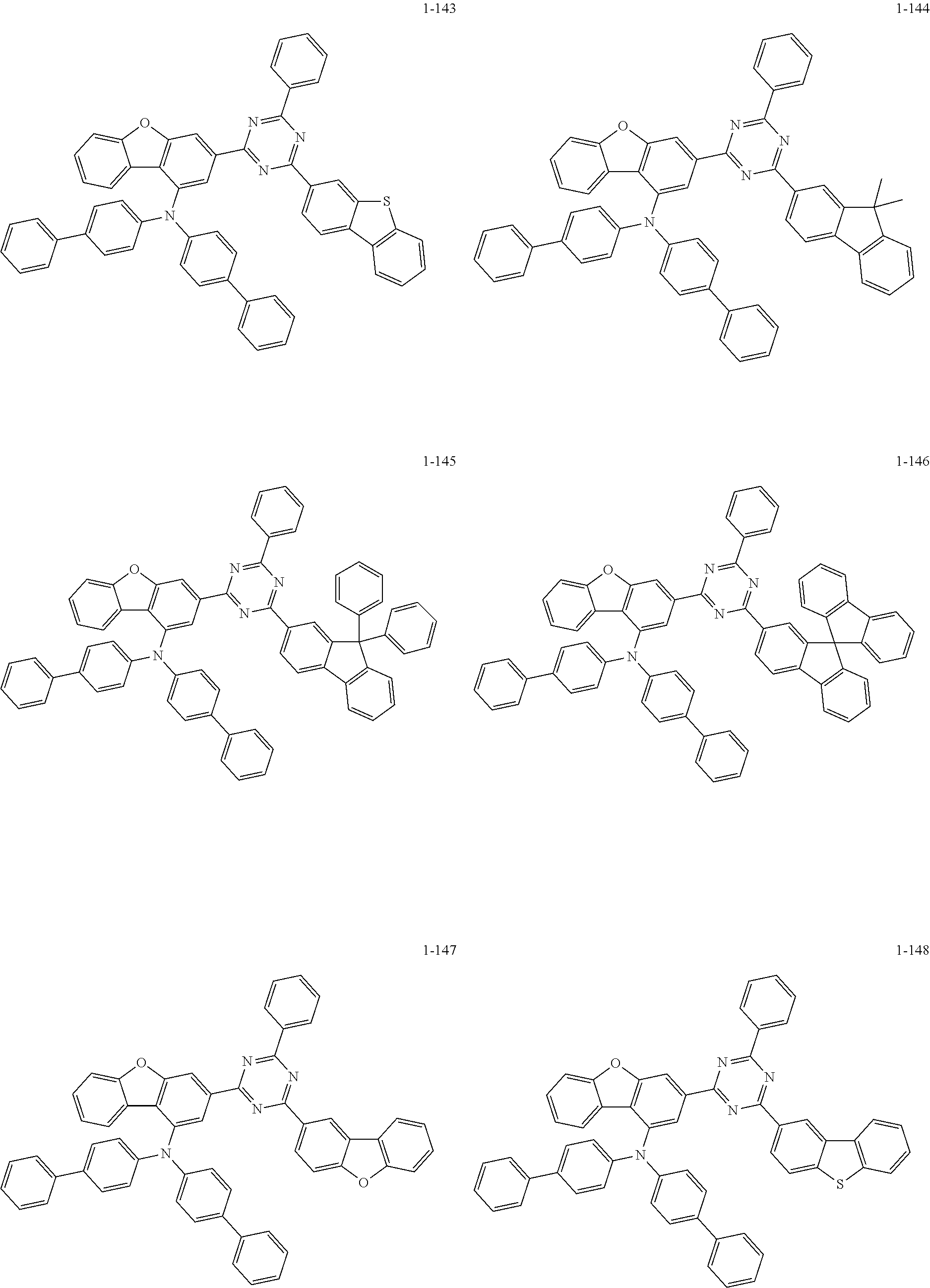

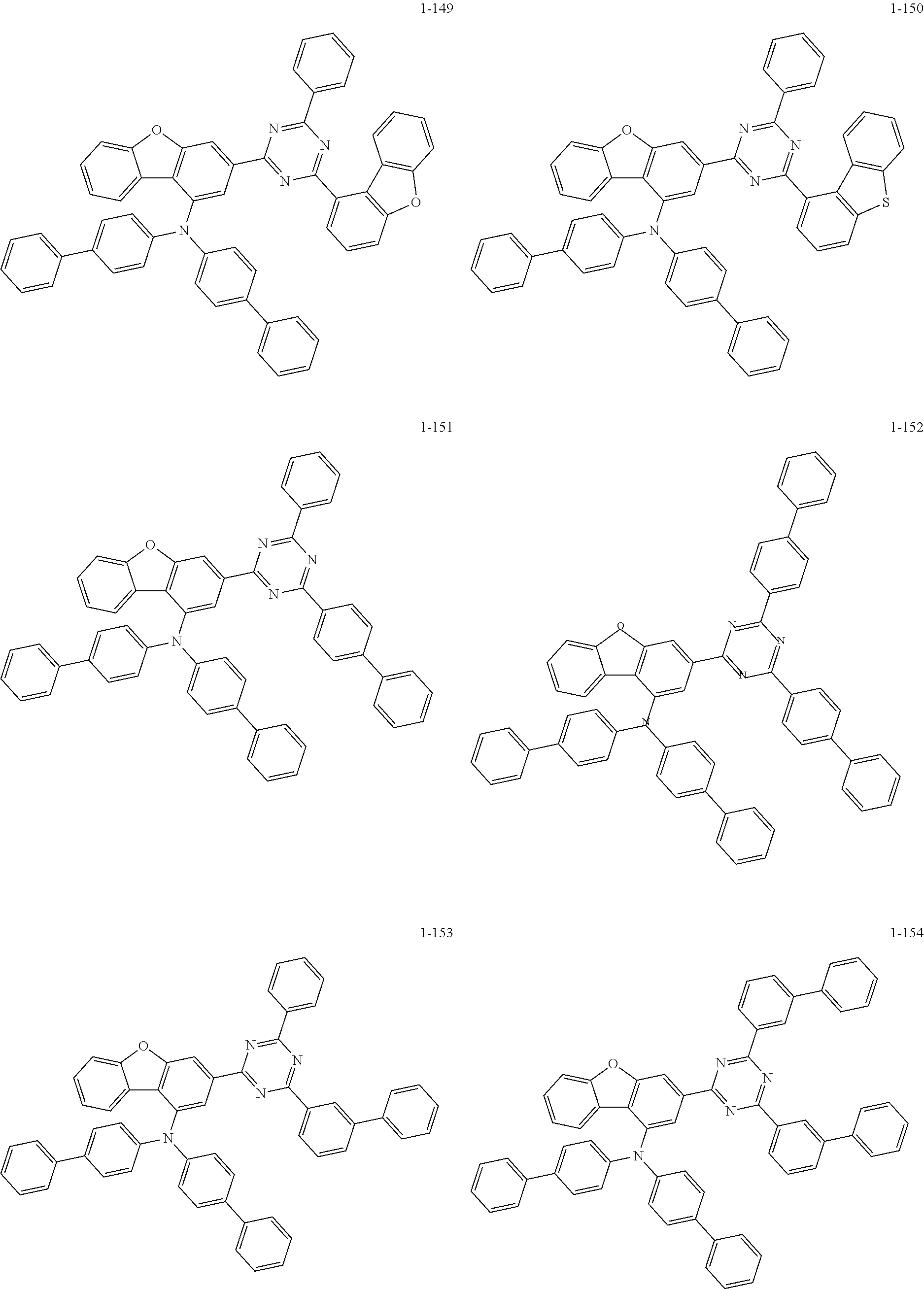

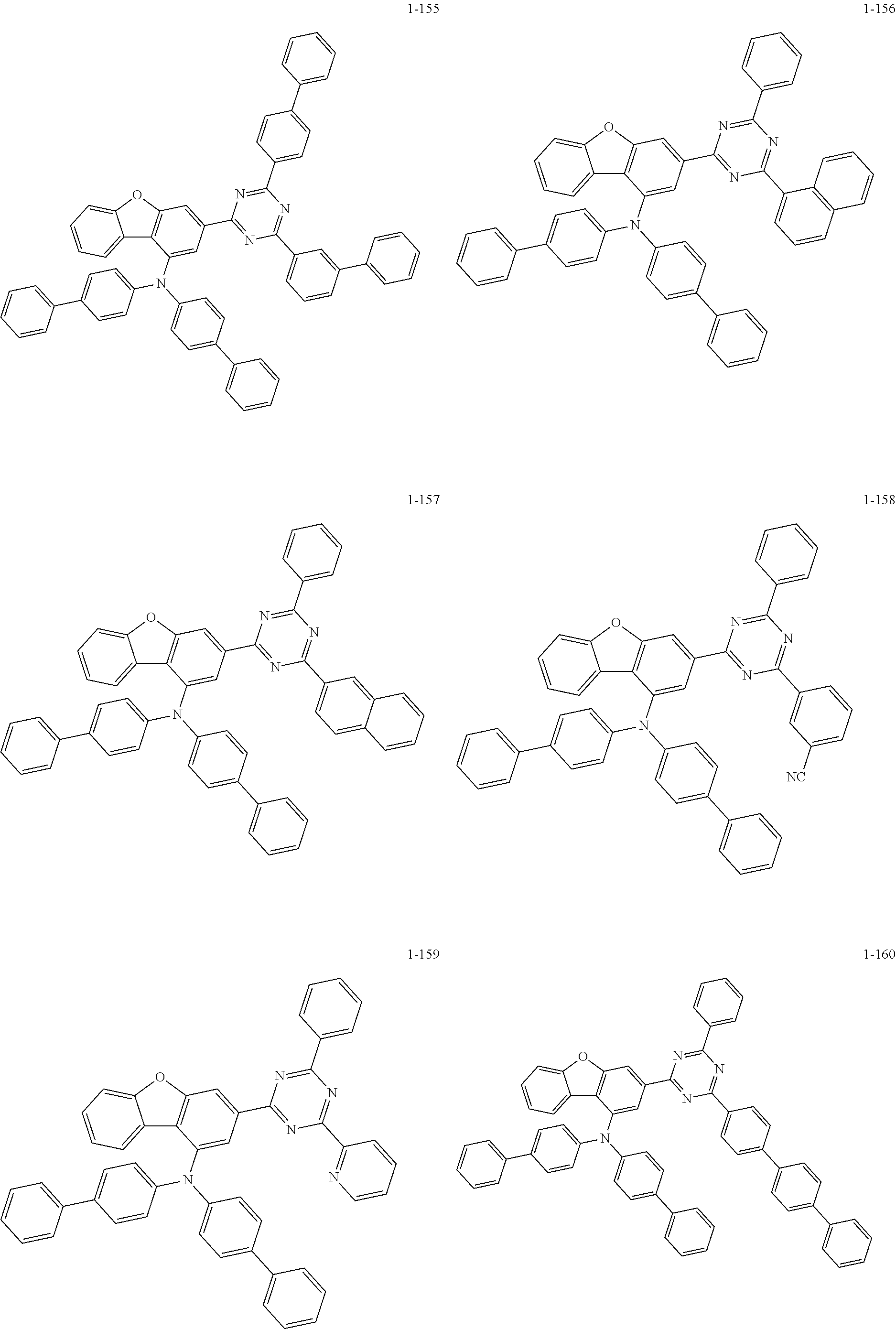

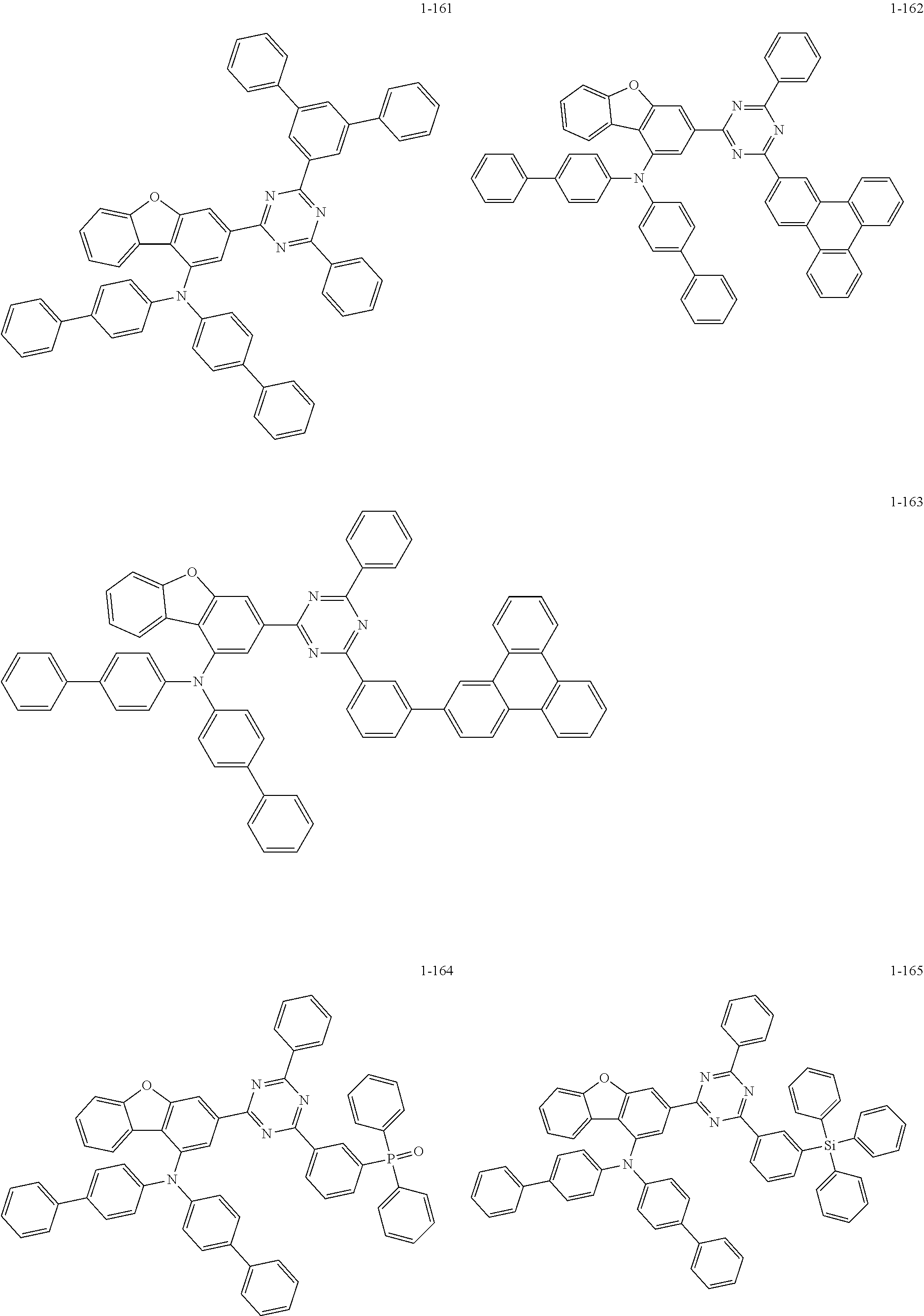

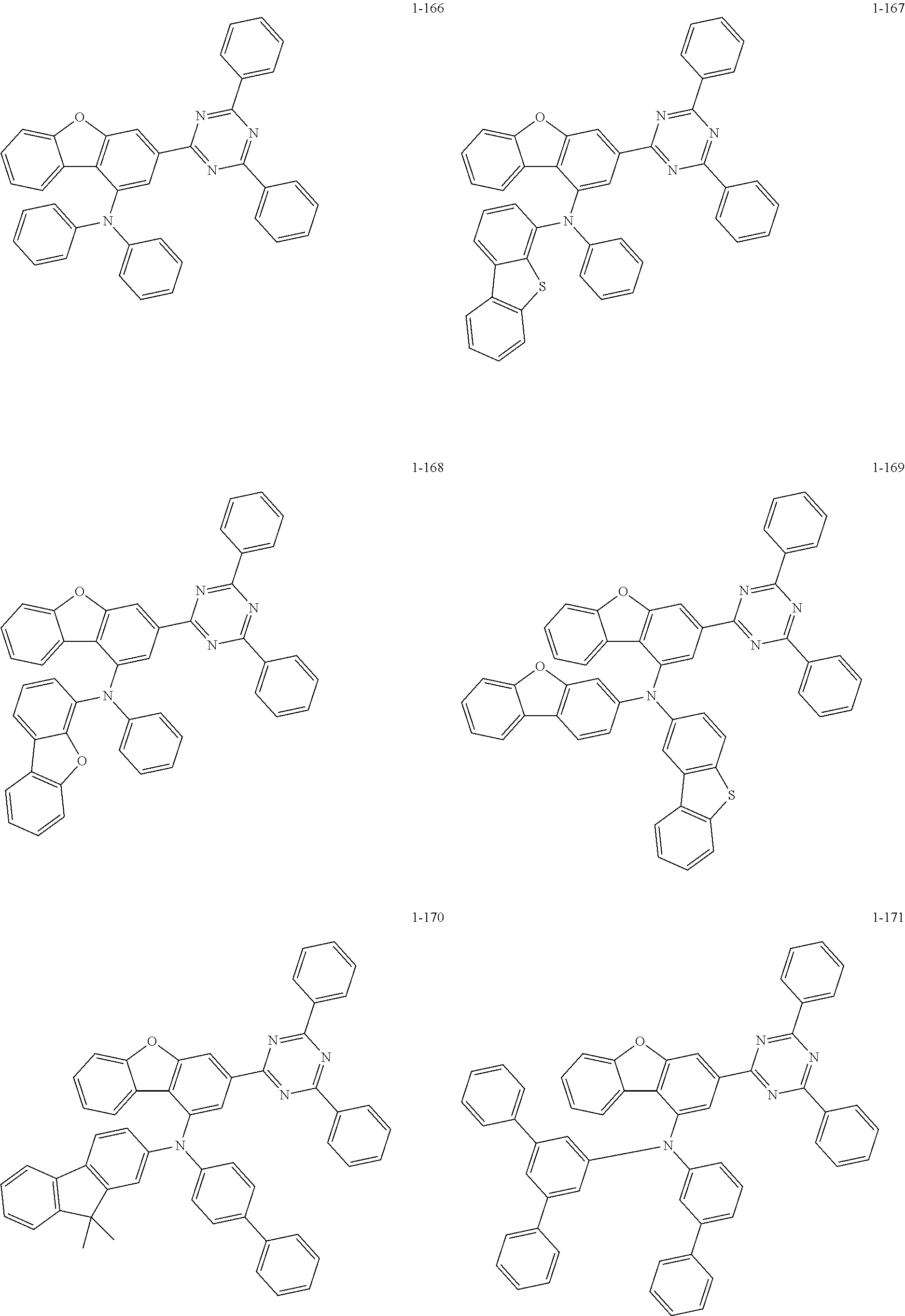

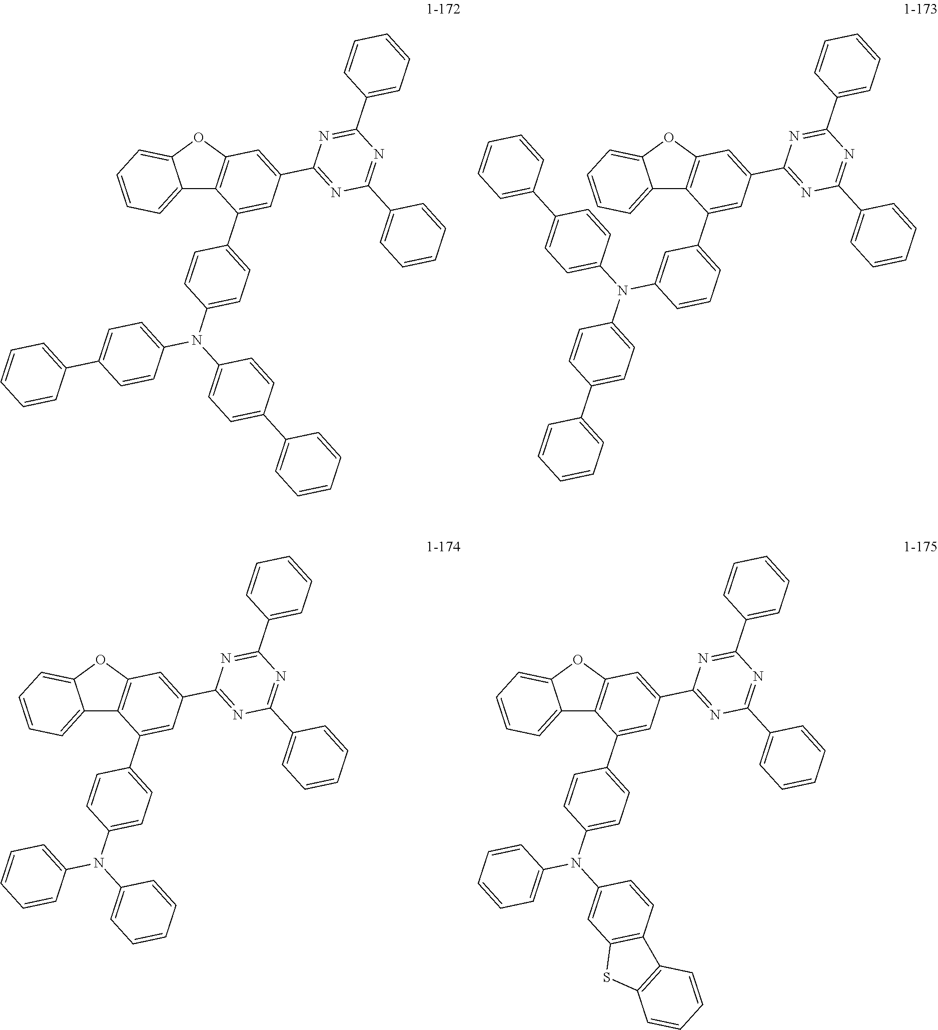 [SMILES: c1ccc(-c2ccc(N(c3ccc(-c4ccccc4)cc3)c3ccc(-c4cc(-c5nc(-c6ccccc6)nc(-c6ccccc6)n5)cc5oc6ccccc6c45)cc3)cc2)cc1 ; c1ccc(-c2ccc(N(c3ccc(-c4ccccc4)cc3)c3cccc(-c4cc(-c5nc(-c6ccccc6)nc(-c6ccccc6)n5)cc5oc6ccccc6c45)c3)cc2)cc1 ; c1ccc(-c2nc(-c3ccccc3)nc(-c3cc(-c4ccc(N(c5ccccc5)c5ccccc5)cc4)c4c(c3)oc3ccccc34)n2)cc1 ; c1ccc(-c2nc(-c3ccccc3)nc(-c3cc(-c4ccc(N(c5ccccc5)c5ccc6c(c5)sc5ccccc56)cc4)c4c(c3)oc3ccccc34)n2)cc1]

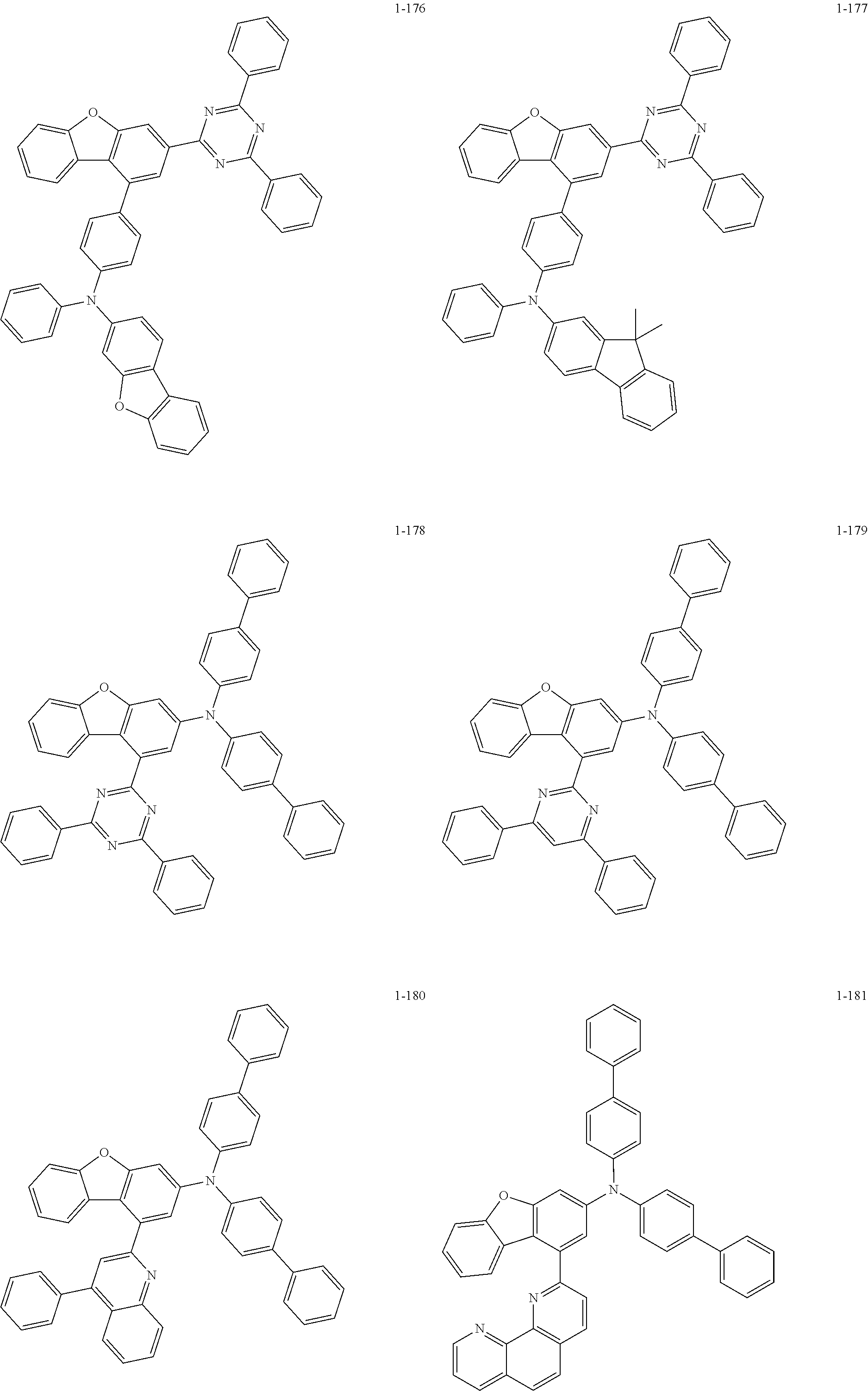

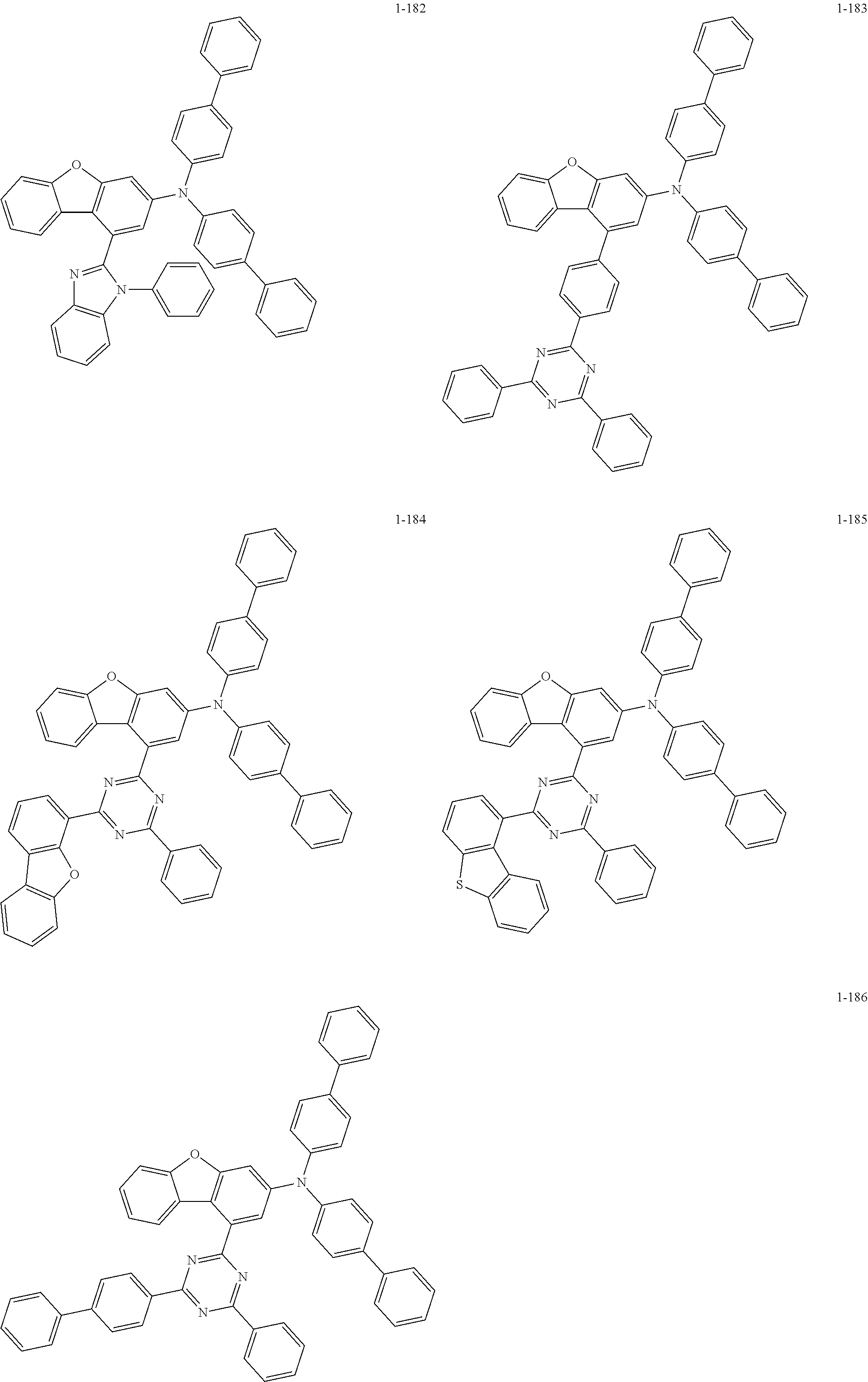

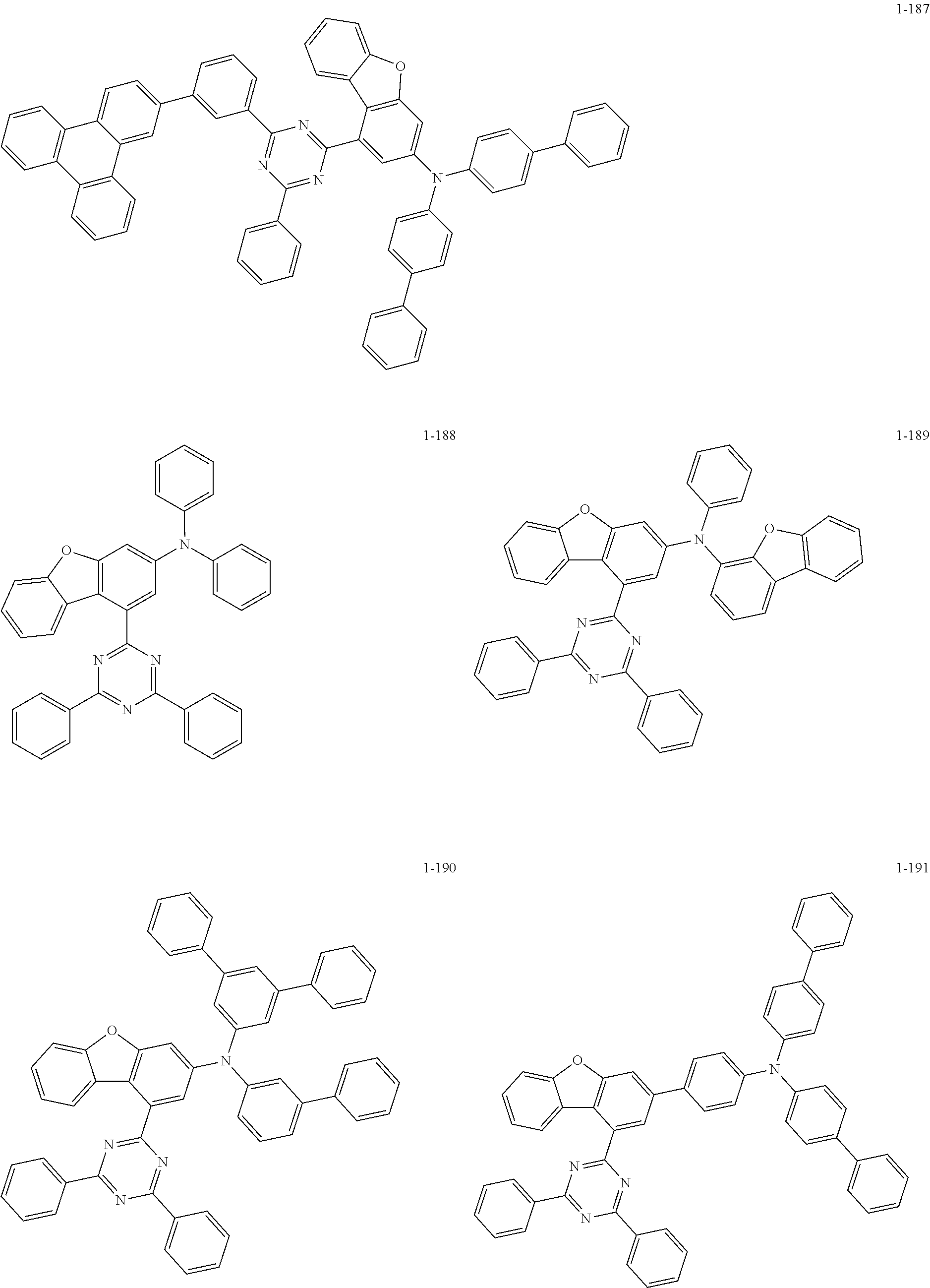

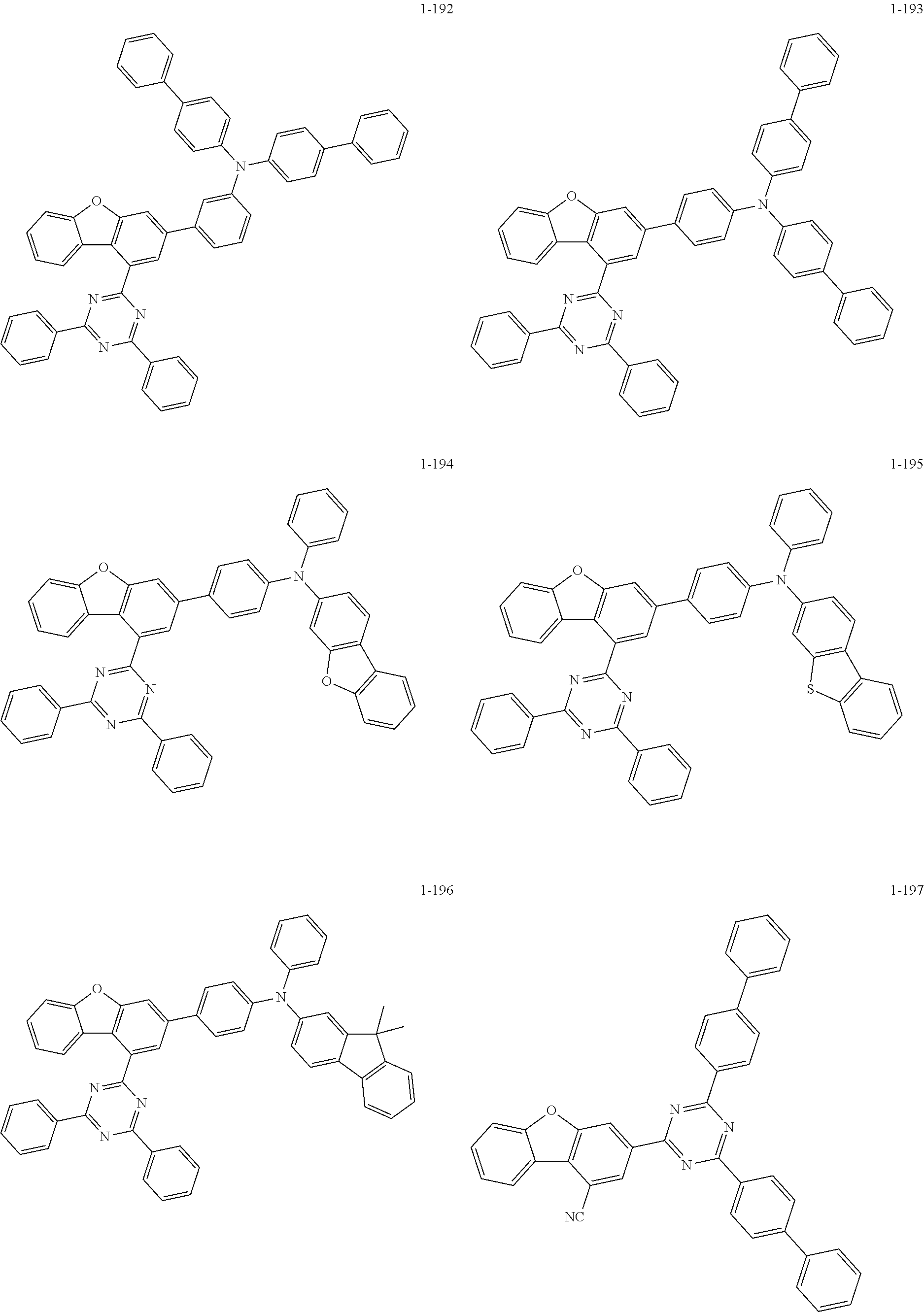

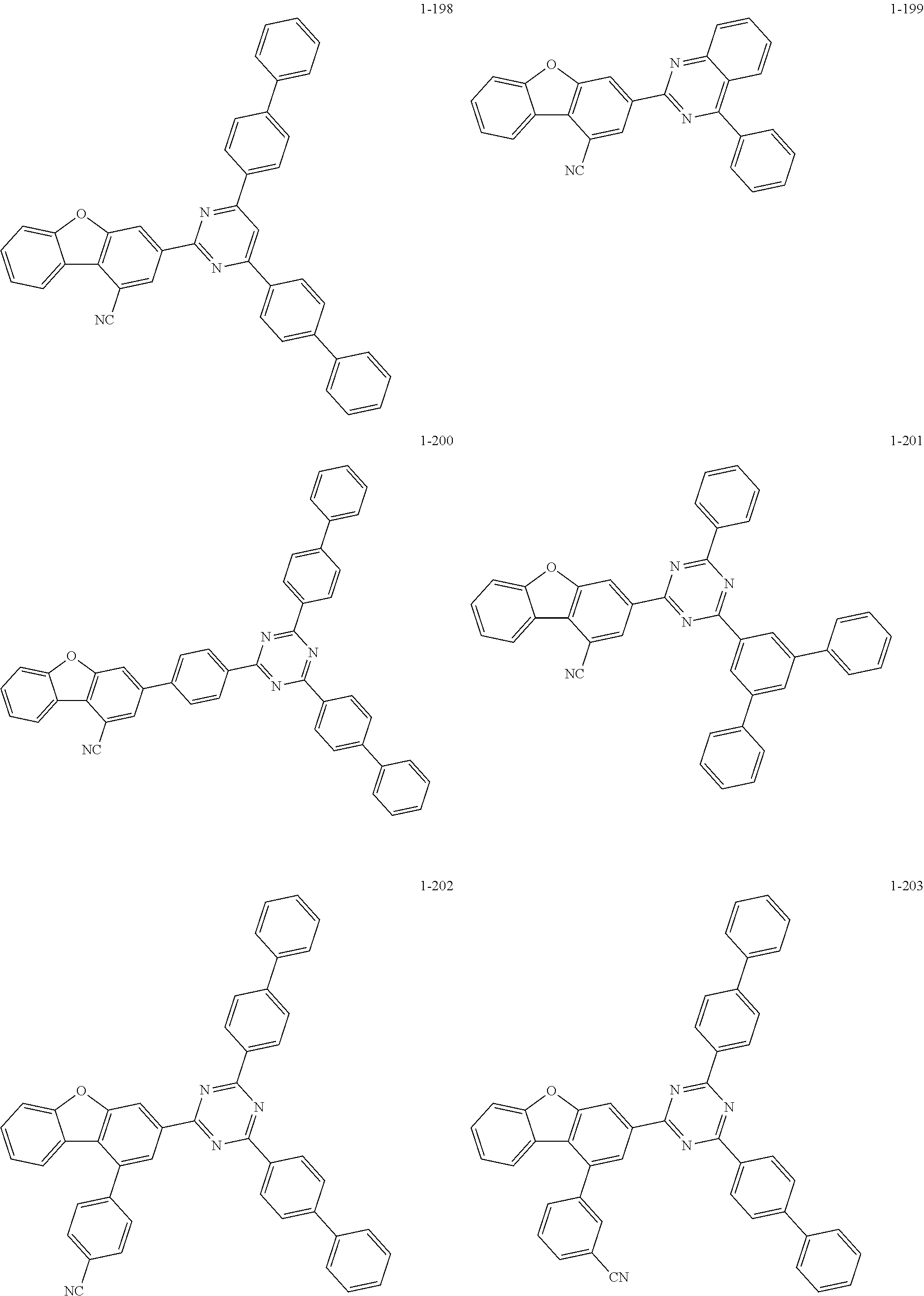 [SMILES: N#Cc1cc(-c2nc(-c3ccc(-c4ccccc4)cc3)cc(-c3ccc(-c4ccccc4)cc3)n2)cc2oc3ccccc3c12 ; N#Cc1cc(-c2nc(-c3ccccc3)c3ccccc3n2)cc2oc3ccccc3c12 ; N#Cc1cc(-c2ccc(-c3nc(-c4ccc(-c5ccccc5)cc4)nc(-c4ccc(-c5ccccc5)cc4)n3)cc2)cc2oc3ccccc3c12 ; N#Cc1cc(-c2nc(-c3ccccc3)nc(-c3cc(-c4ccccc4)cc(-c4ccccc4)c3)n2)cc2oc3ccccc3c12 ; N#Cc1ccc(-c2cc(-c3nc(-c4ccc(-c5ccccc5)cc4)nc(-c4ccc(-c5ccccc5)cc4)n3)cc3oc4ccccc4c23)cc1 ; N#Cc1cccc(-c2cc(-c3nc(-c4ccc(-c5ccccc5)cc4)nc(-c4ccc(-c5ccccc5)cc4)n3)cc3oc4ccccc4c23)c1]

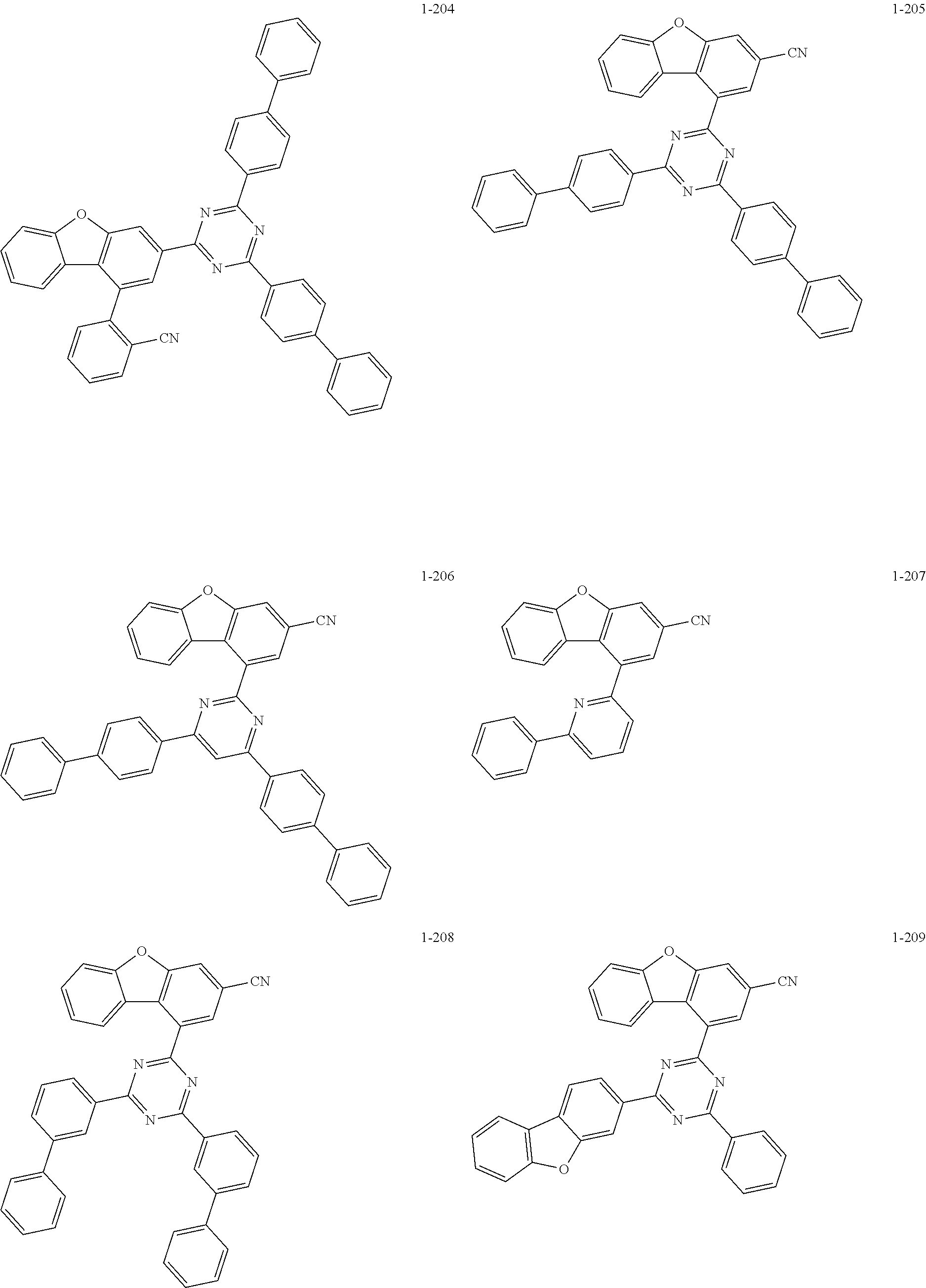 [SMILES: N#Cc1ccccc1-c1cc(-c2nc(-c3ccc(-c4ccccc4)cc3)nc(-c3ccc(-c4ccccc4)cc3)n2)cc2oc3ccccc3c12 ; N#Cc1cc(-c2nc(-c3ccc(-c4ccccc4)cc3)nc(-c3ccc(-c4ccccc4)cc3)n2)c2c(c1)oc1ccccc12 ; N#Cc1cc(-c2nc(-c3ccc(-c4ccccc4)cc3)cc(-c3ccc(-c4ccccc4)cc3)n2)c2c(c1)oc1ccccc12 ; N#Cc1cc(-c2cccc(-c3ccccc3)n2)c2c(c1)oc1ccccc12 ; N#Cc1cc(-c2nc(-c3cccc(-c4ccccc4)c3)nc(-c3cccc(-c4ccccc4)c3)n2)c2c(c1)oc1ccccc12 ; N#Cc1cc(-c2nc(-c3ccccc3)nc(-c3ccc4c(c3)oc3ccccc34)n2)c2c(c1)oc1ccccc12]

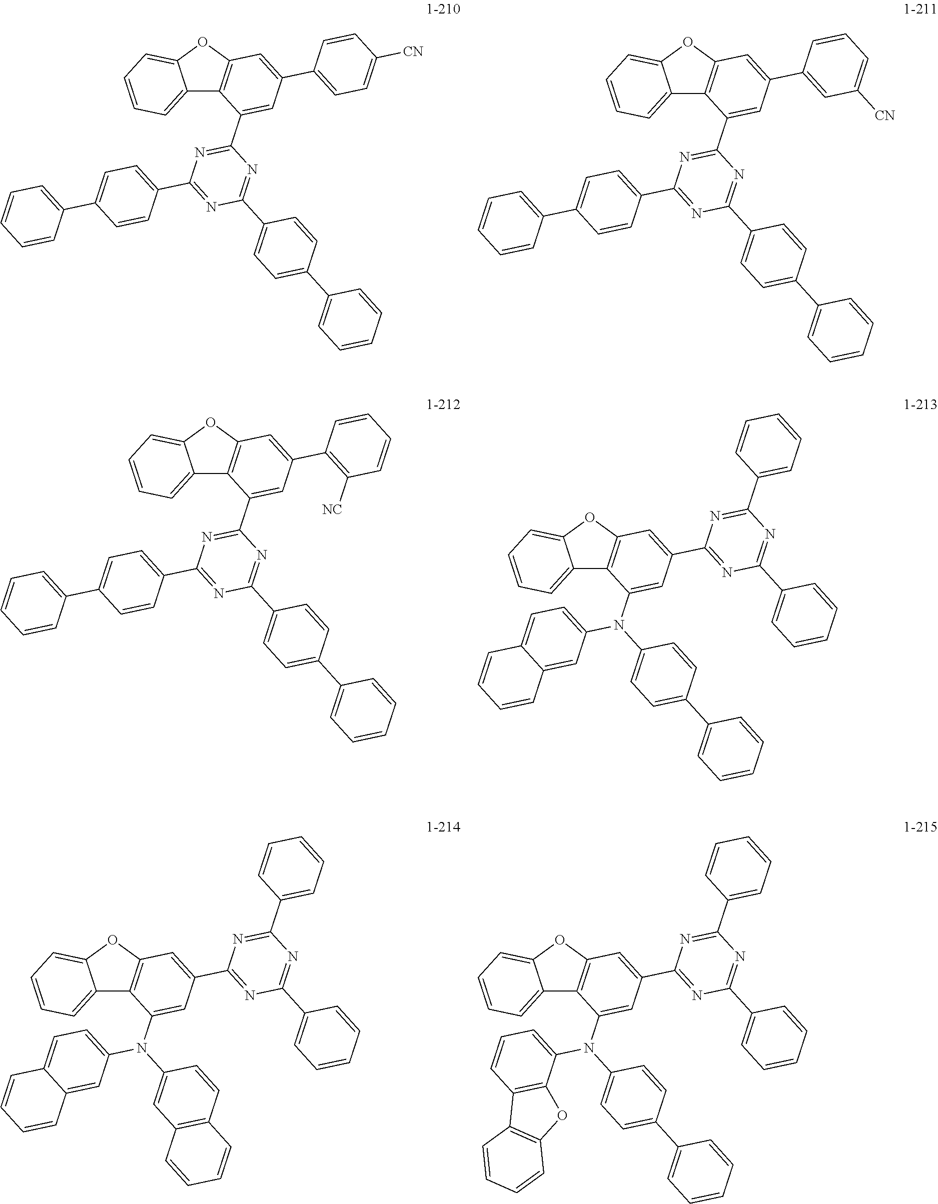

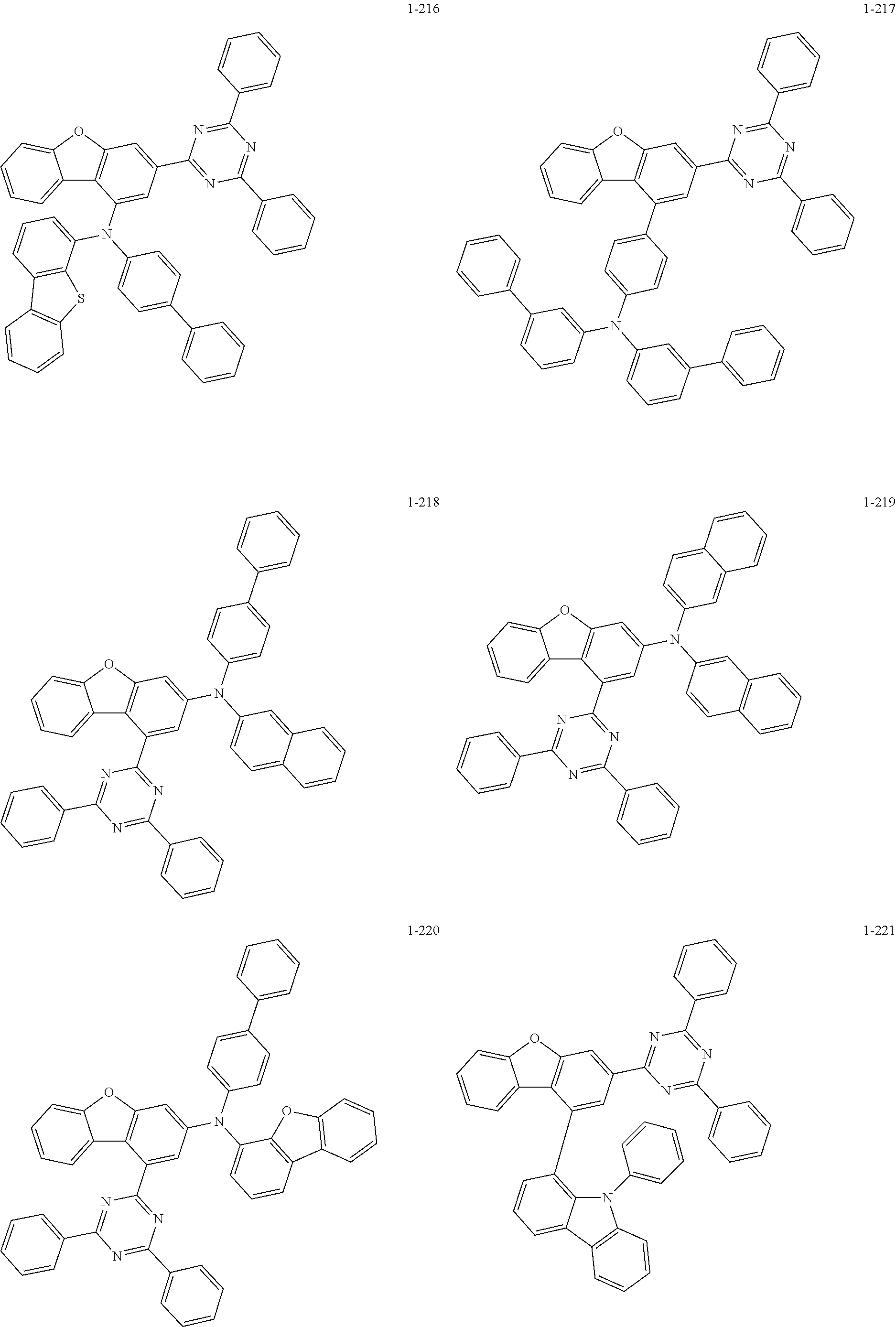

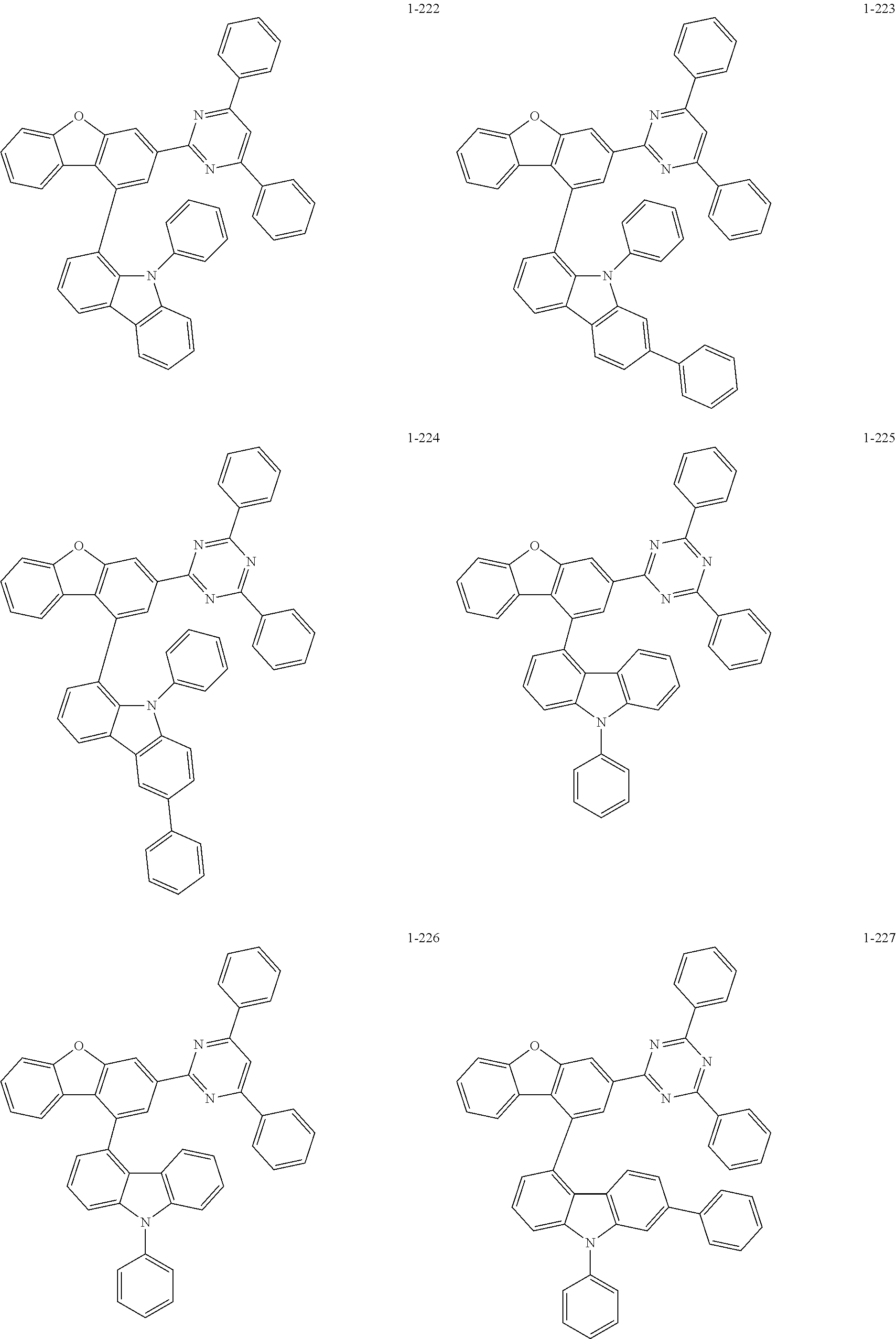

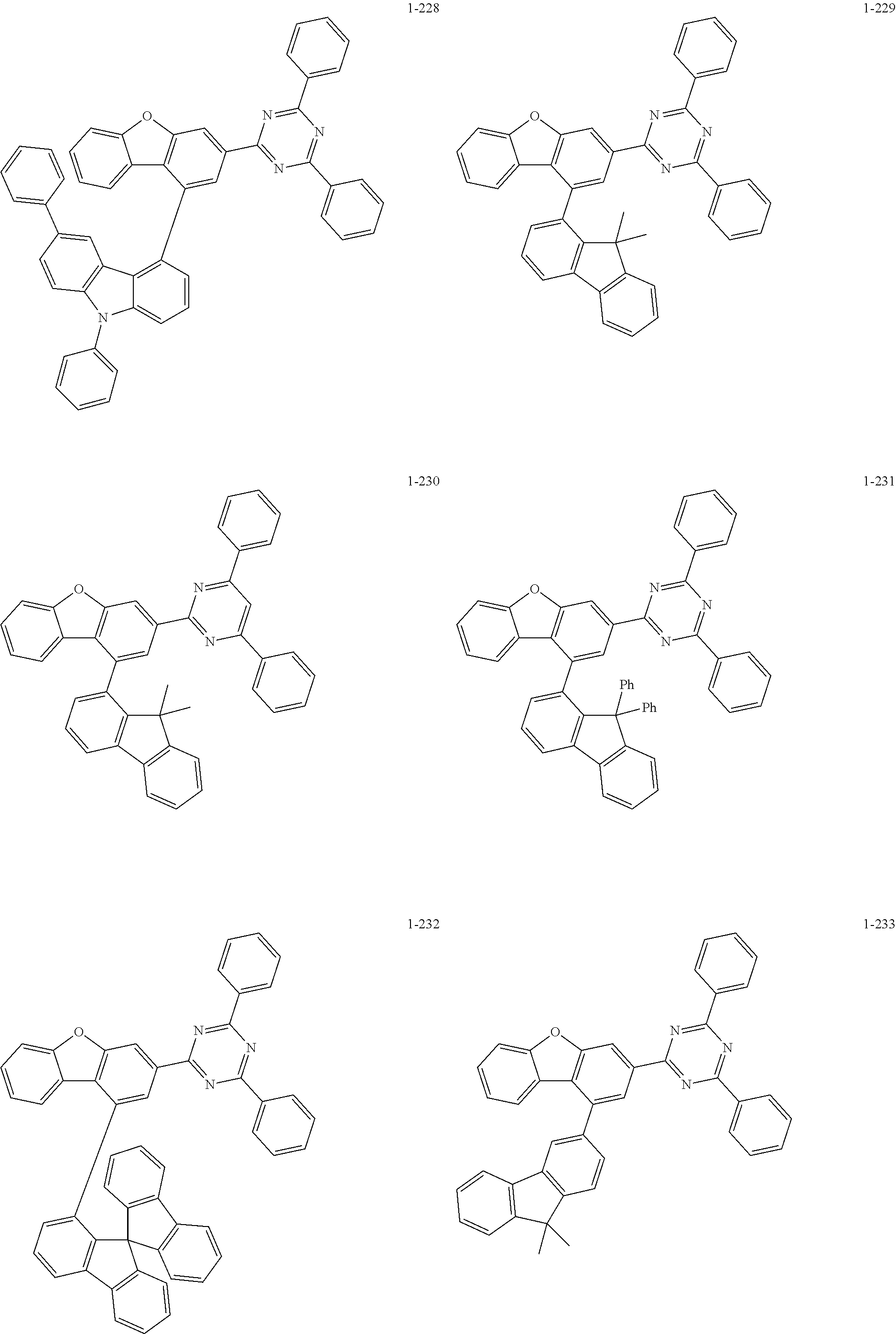

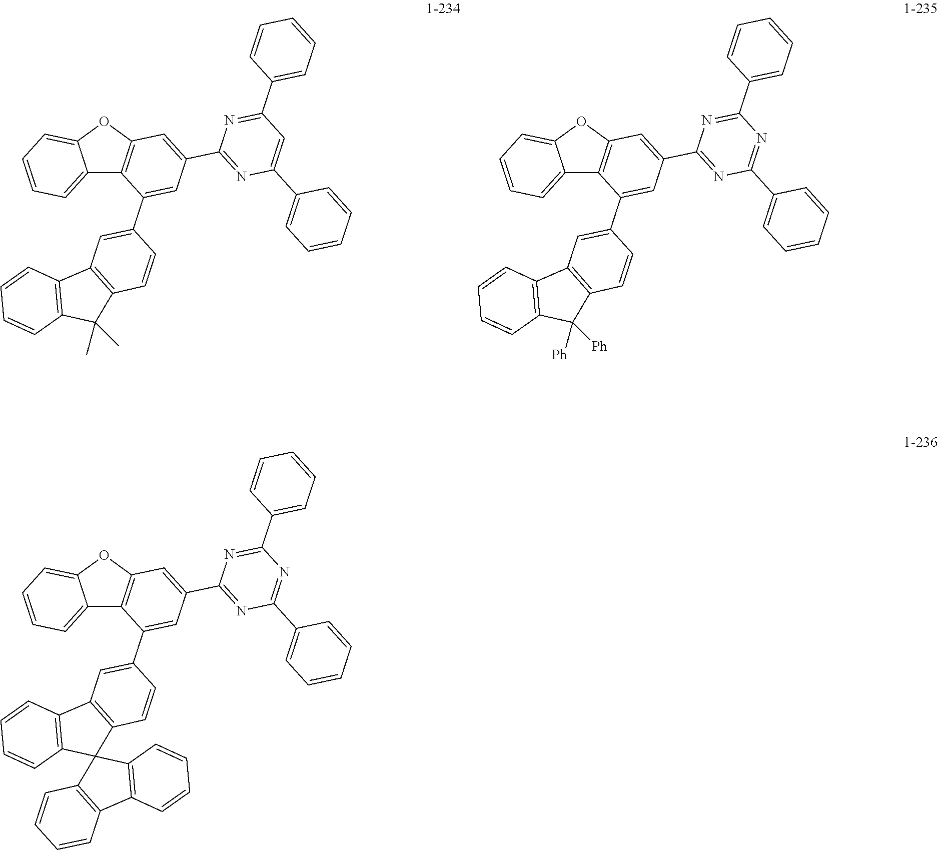 [SMILES: CC1(C)c2ccccc2-c2cc(-c3cc(-c4nc(-c5ccccc5)cc(-c5ccccc5)n4)cc4oc5ccccc5c34)ccc21 ; c1ccc(-c2nc(-c3ccccc3)nc(-c3cc(-c4ccc5c(c4)-c4ccccc4C5(c4ccccc4)c4ccccc4)c4c(c3)oc3ccccc34)n2)cc1 ; c1ccc(-c2nc(-c3ccccc3)nc(-c3cc(-c4ccc5c(c4)-c4ccccc4C54c5ccccc5-c5ccccc54)c4c(c3)oc3ccccc34)n2)cc1]

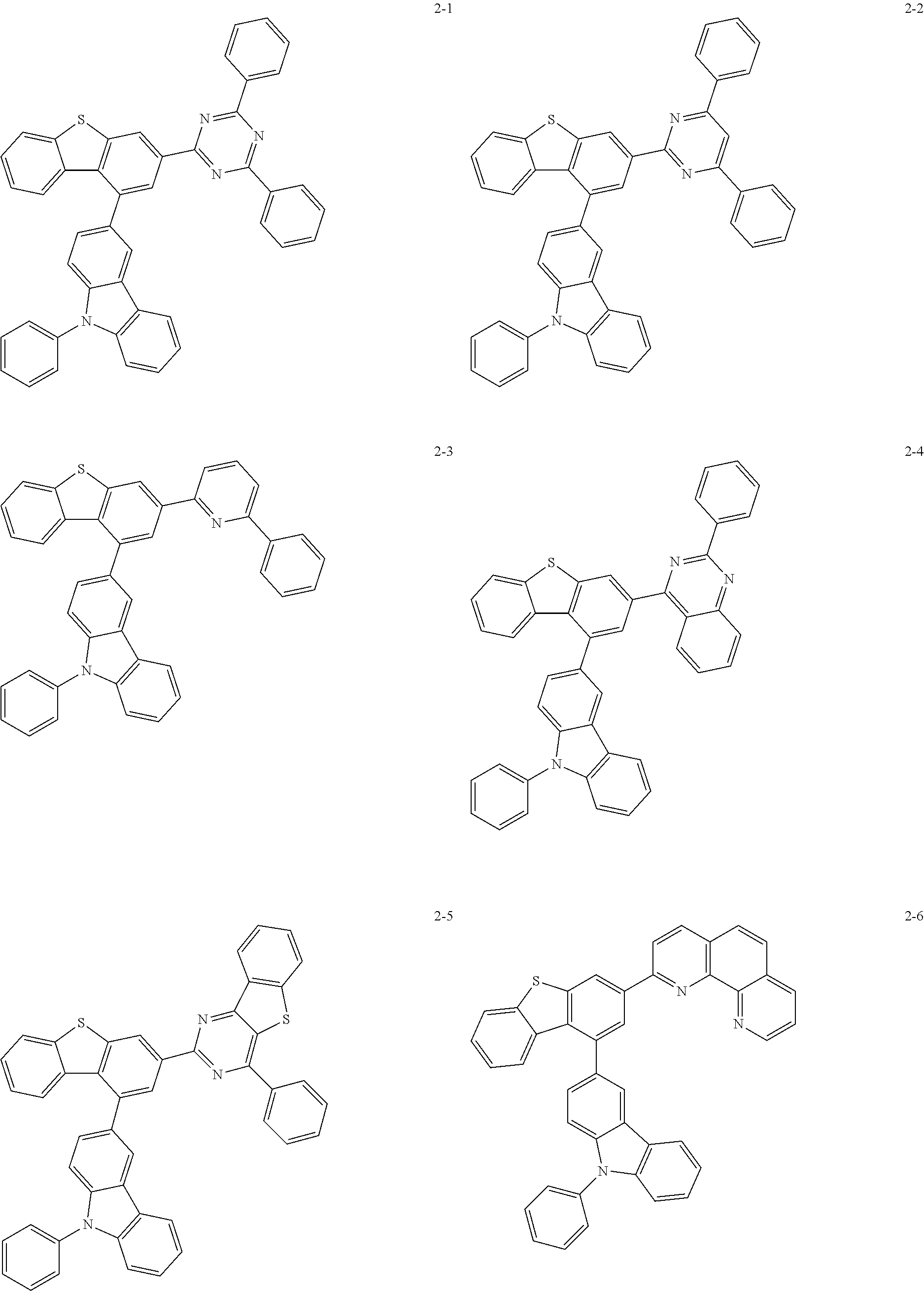

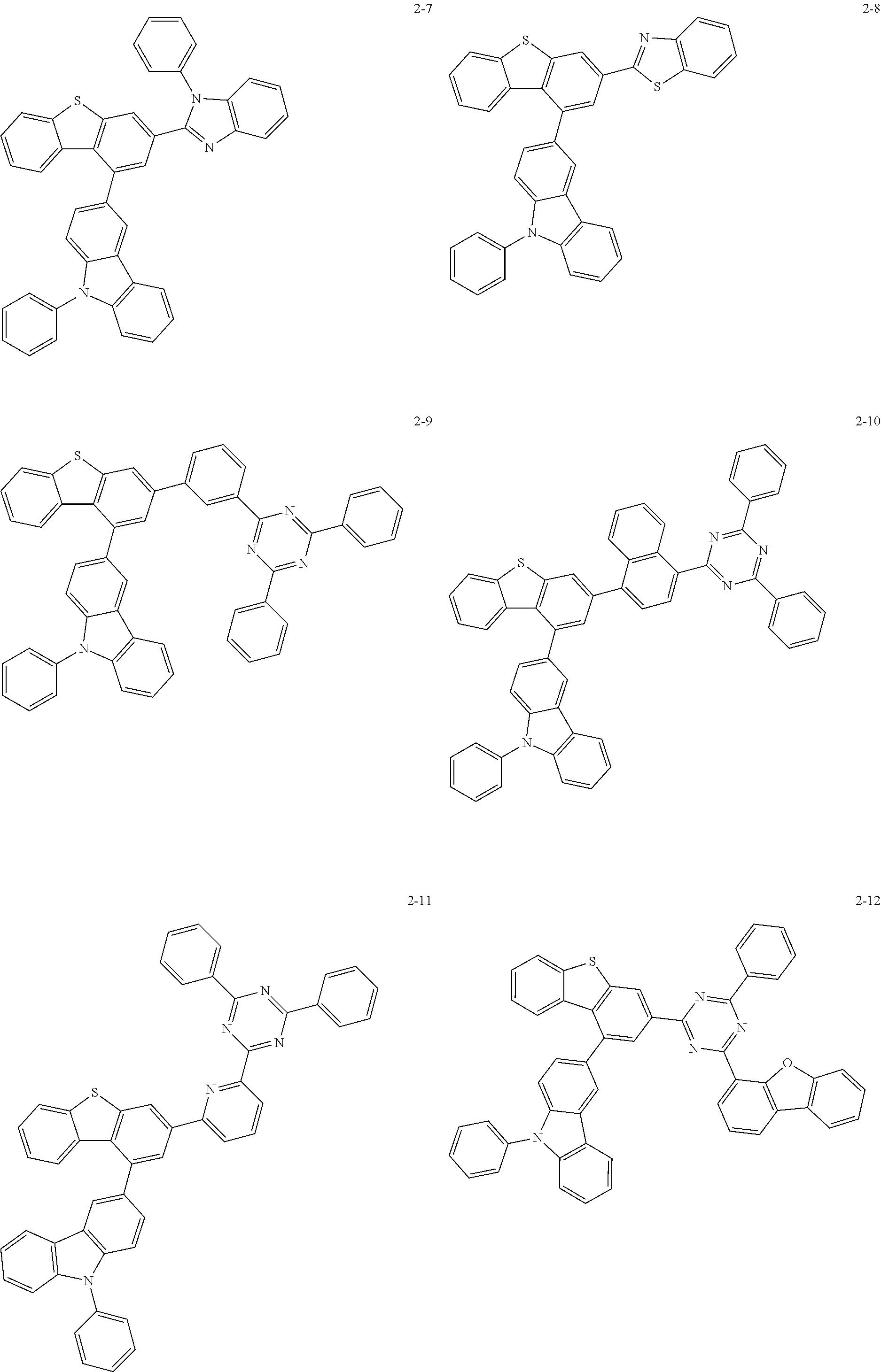

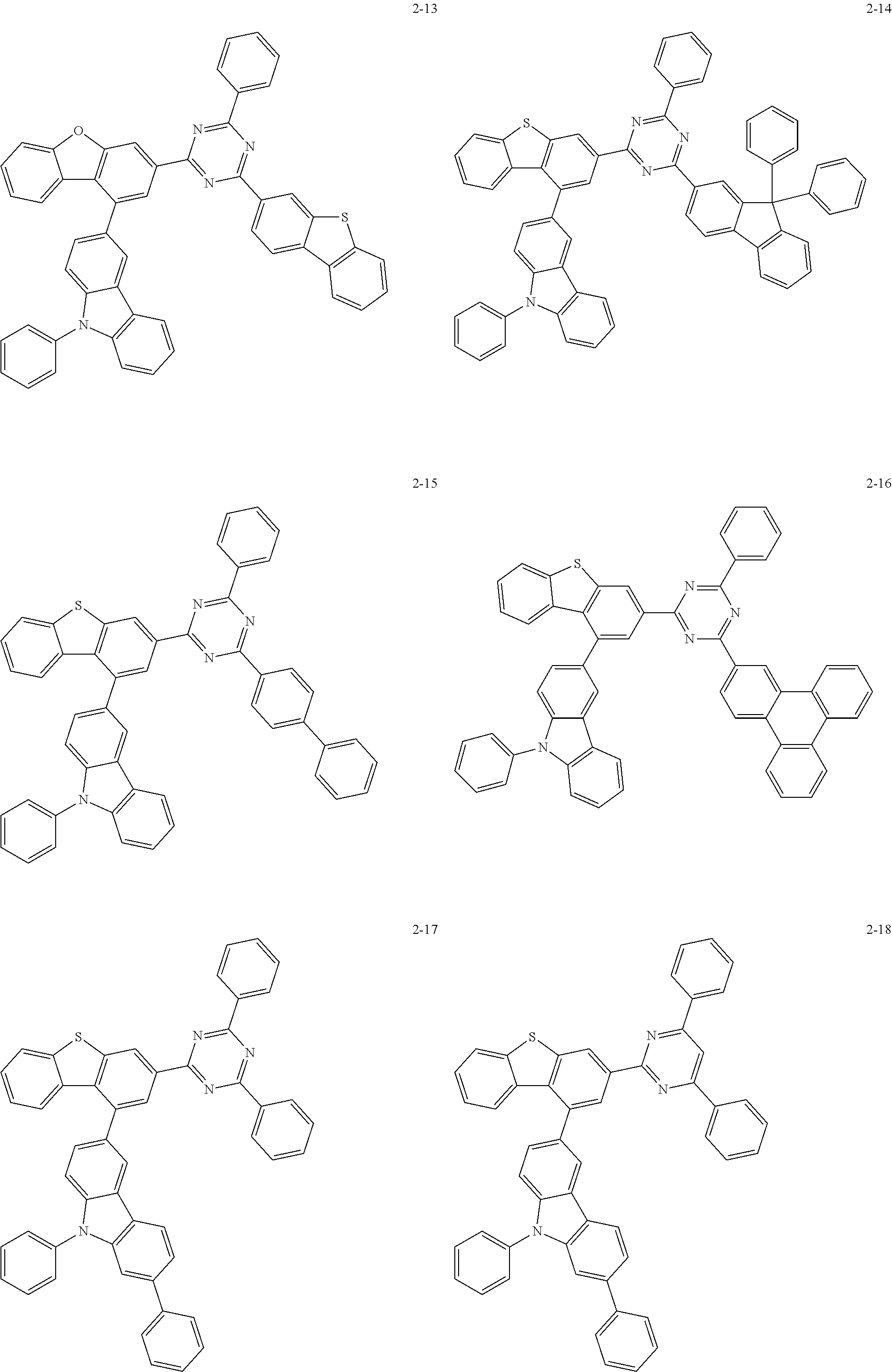

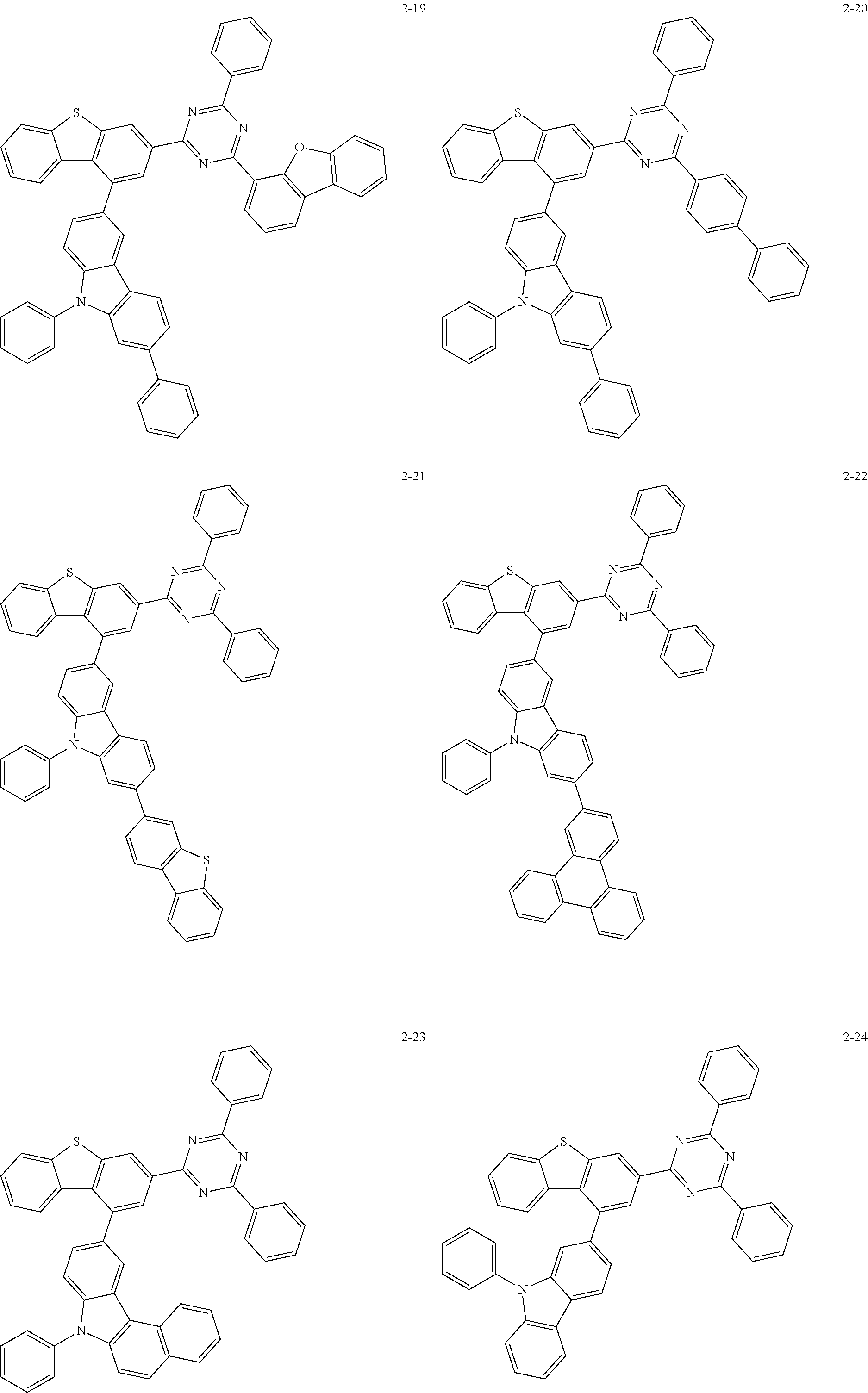

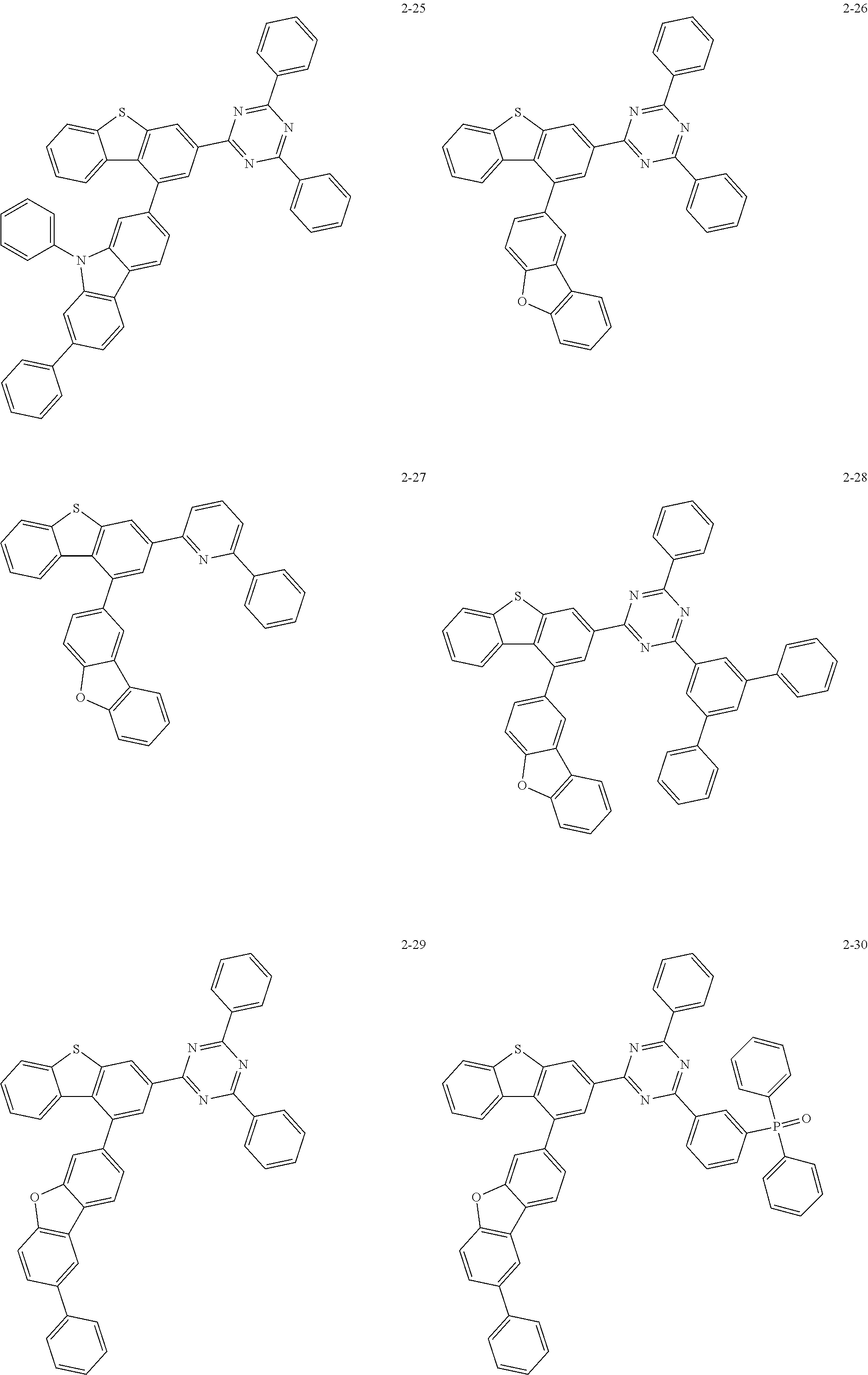

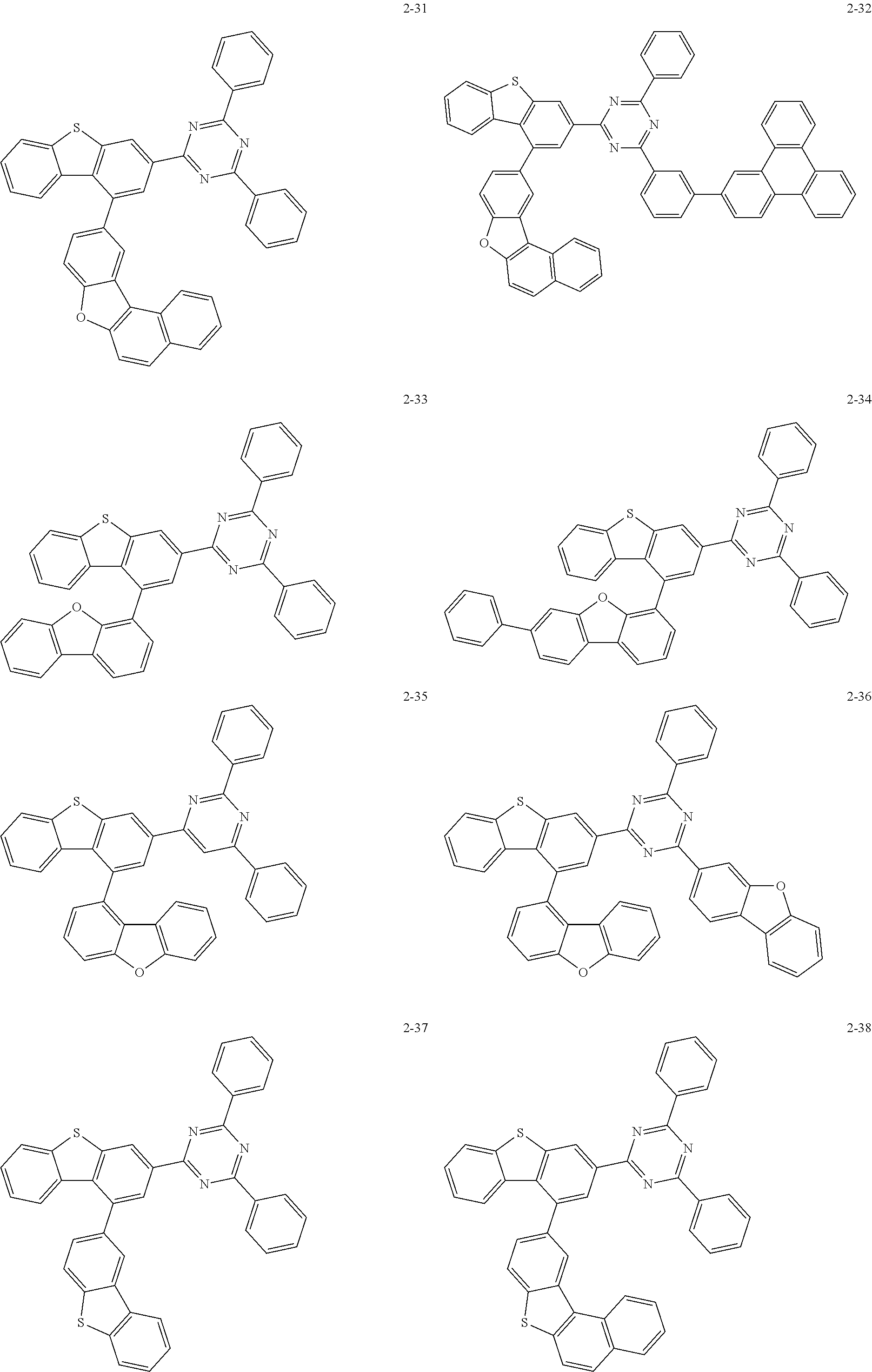

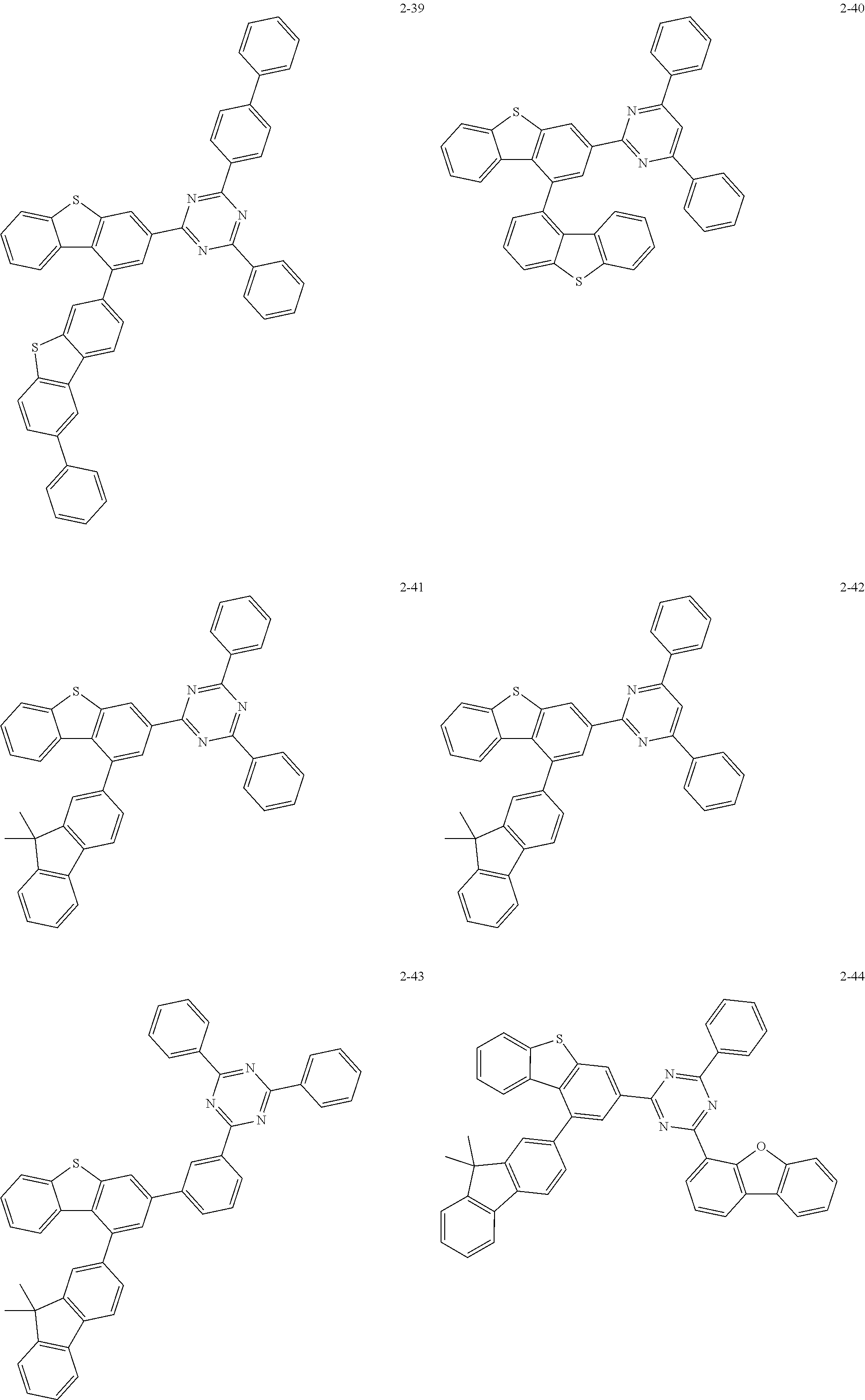

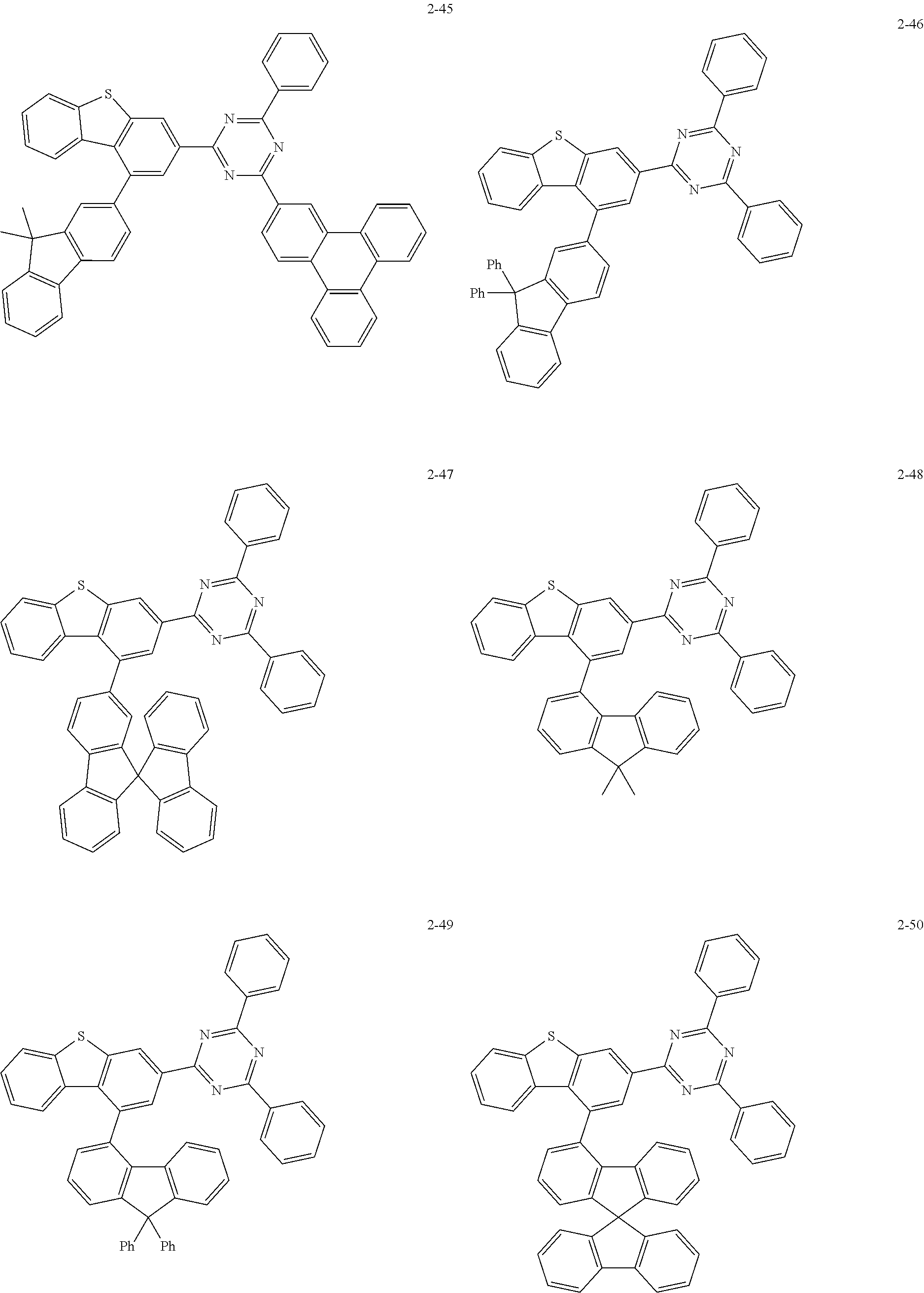

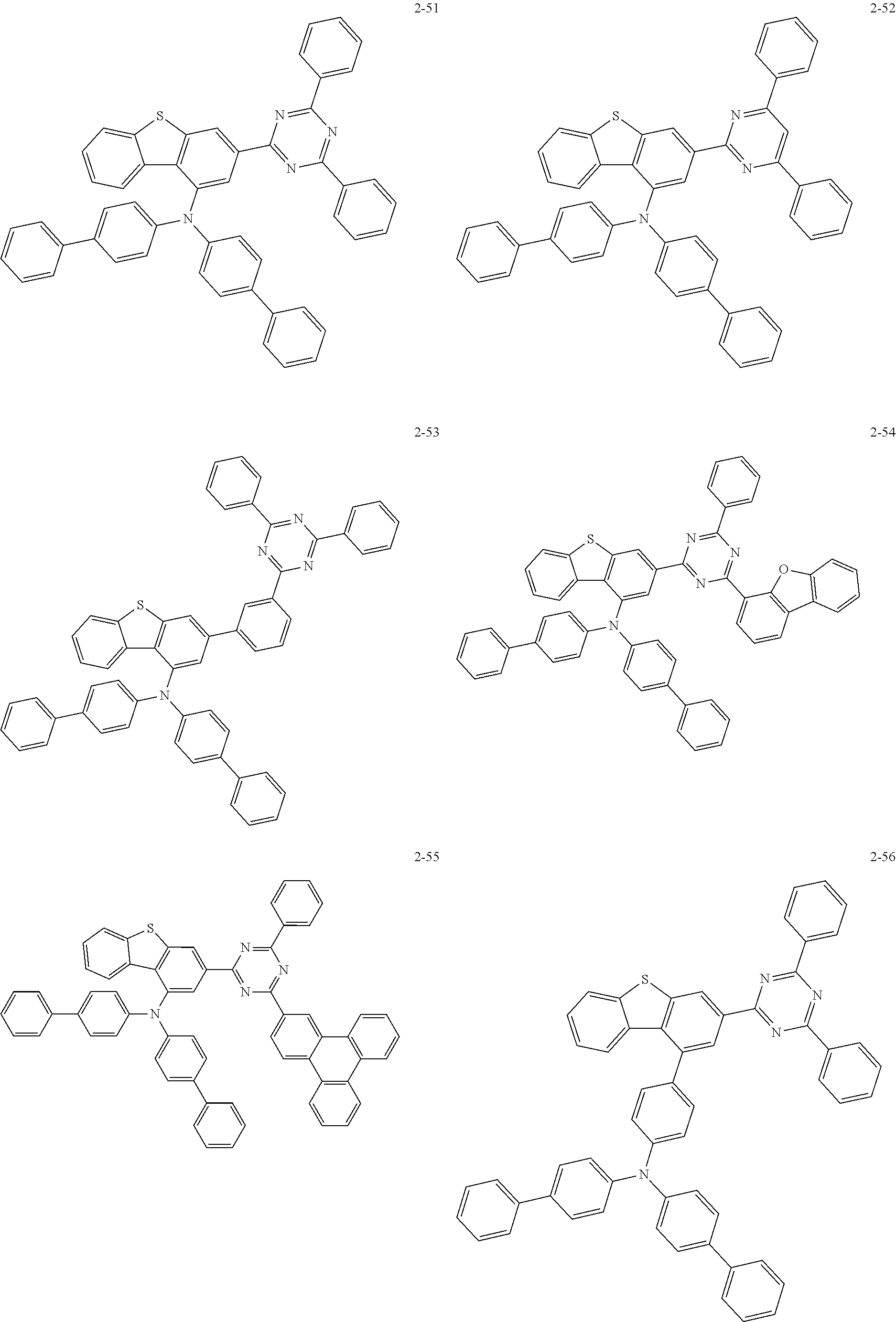

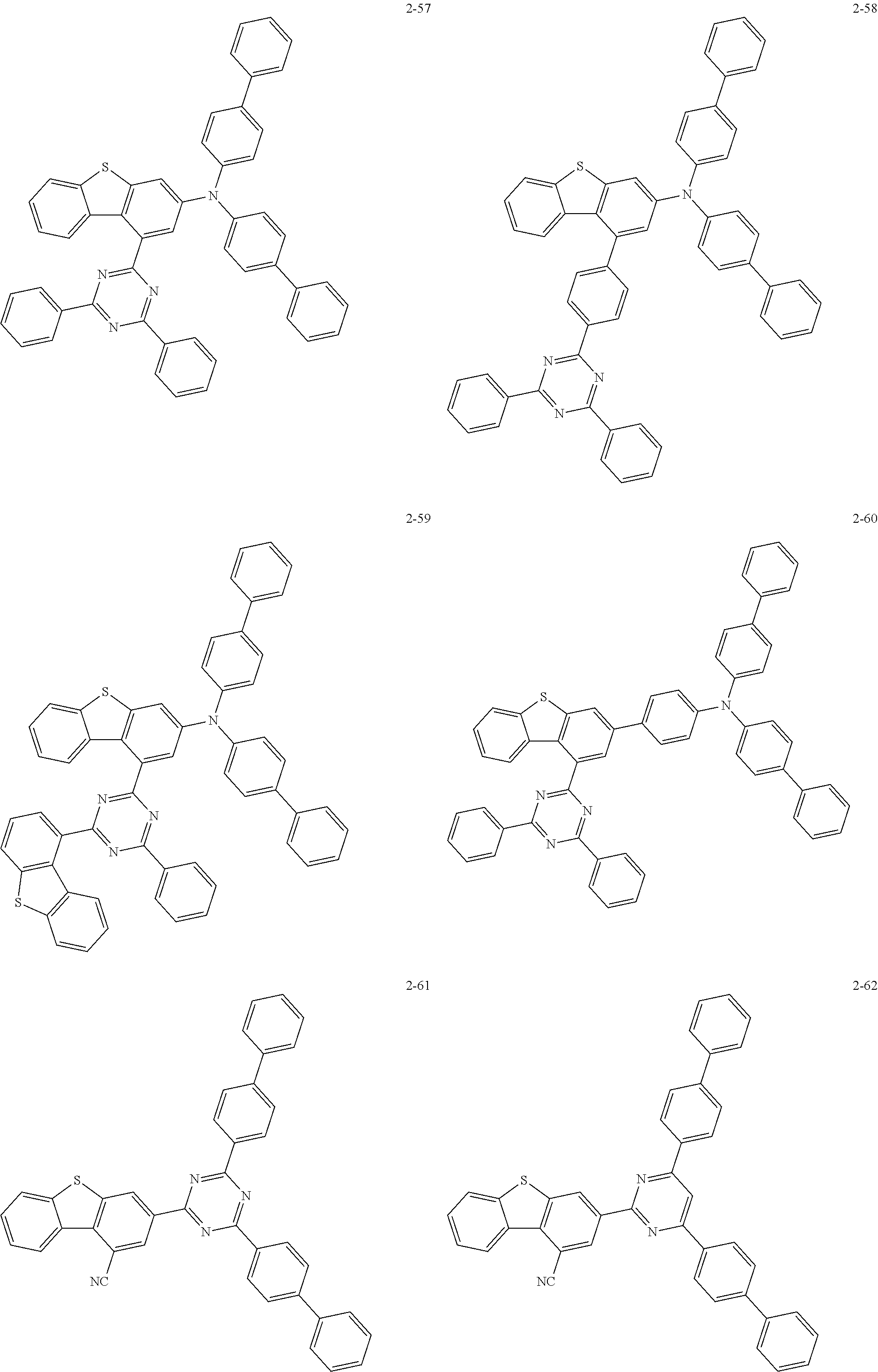

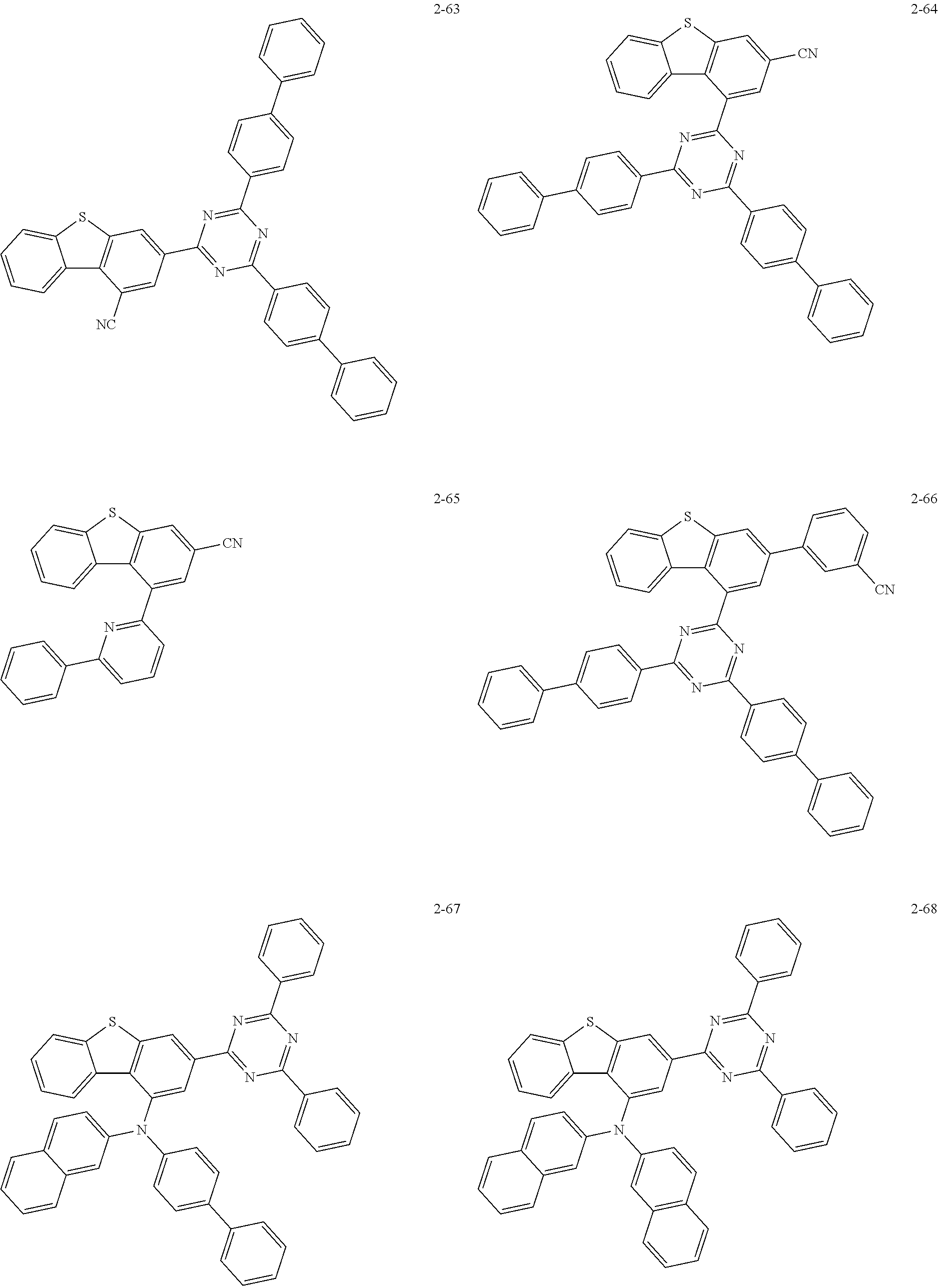 [SMILES: N#Cc1cc(-c2nc(-c3ccc(-c4ccccc4)cc3)nc(-c3ccc(-c4ccccc4)cc3)n2)cc2sc3ccccc3c12 ; N#Cc1cc(-c2nc(-c3ccc(-c4ccccc4)cc3)nc(-c3ccc(-c4ccccc4)cc3)n2)c2c(c1)sc1ccccc12 ; N#Cc1cc(-c2cccc(-c3ccccc3)n2)c2c(c1)sc1ccccc12 ; N#Cc1cccc(-c2cc(-c3nc(-c4ccc(-c5ccccc5)cc4)nc(-c4ccc(-c5ccccc5)cc4)n3)c3c(c2)sc2ccccc23)c1 ; c1ccc(-c2ccc(N(c3ccc4ccccc4c3)c3cc(-c4nc(-c5ccccc5)nc(-c5ccccc5)n4)cc4sc5ccccc5c34)cc2)cc1 ; c1ccc(-c2nc(-c3ccccc3)nc(-c3cc(N(c4ccc5ccccc5c4)c4ccc5ccccc5c4)c4c(c3)sc3ccccc34)n2)cc1]

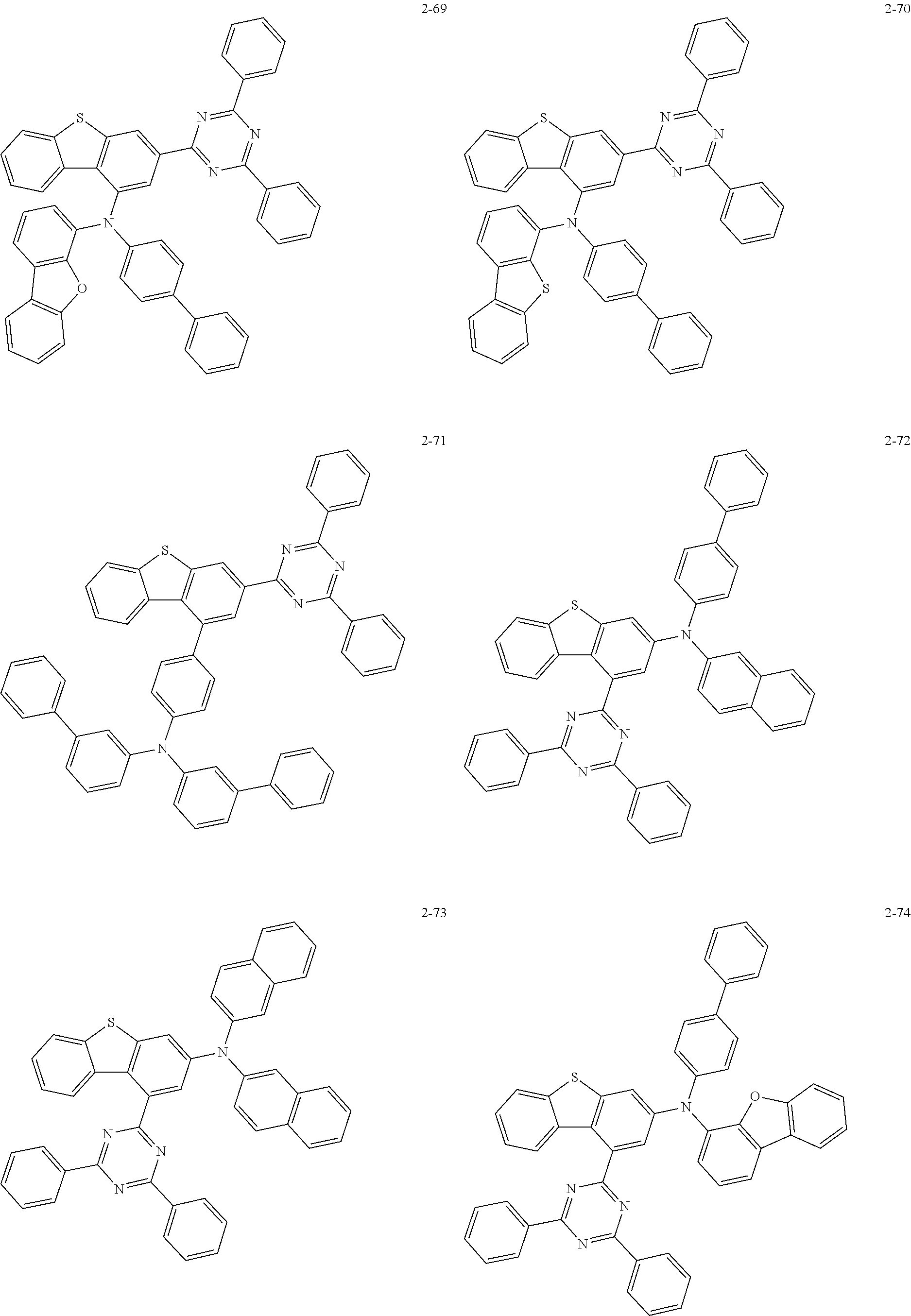

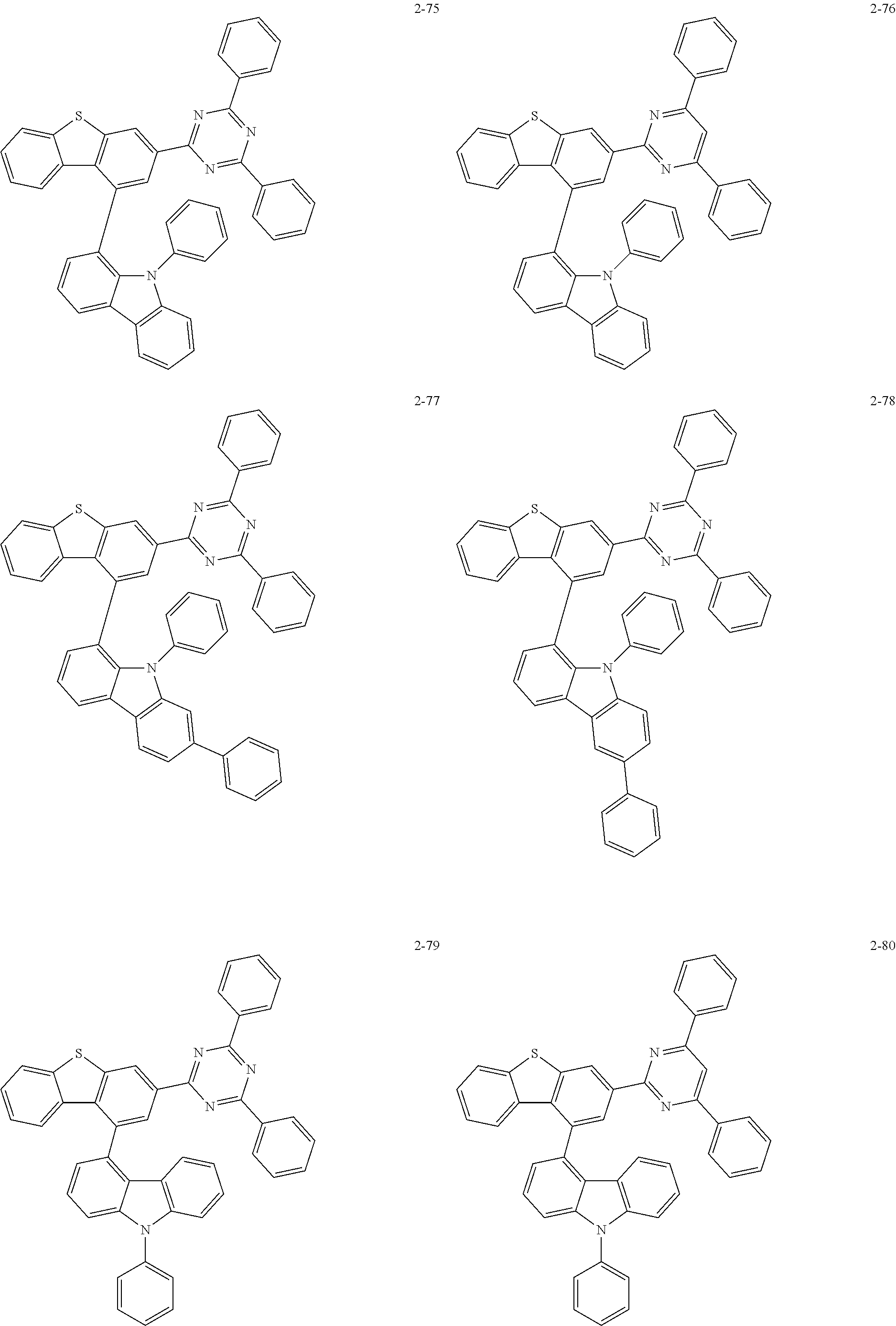

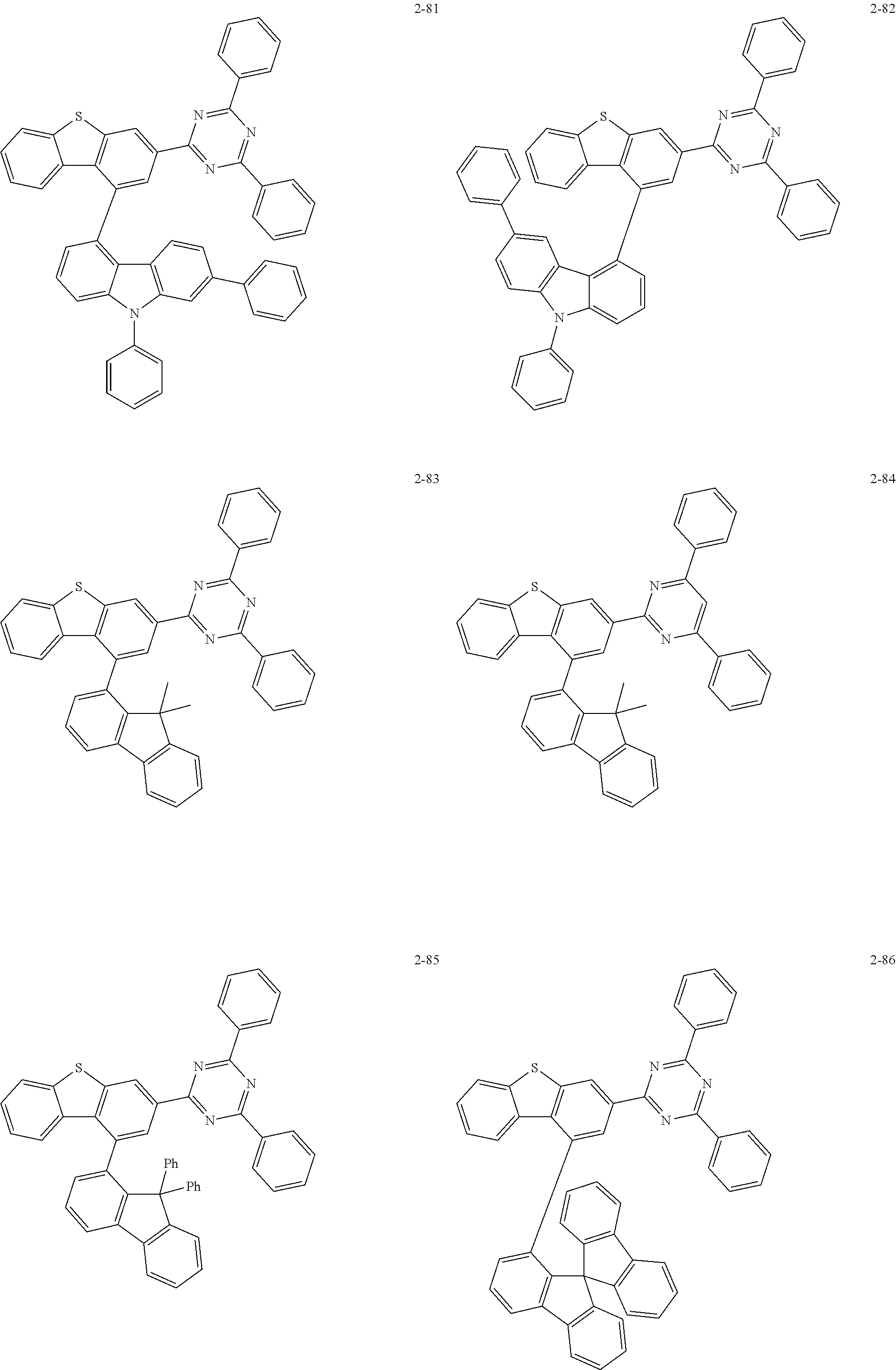

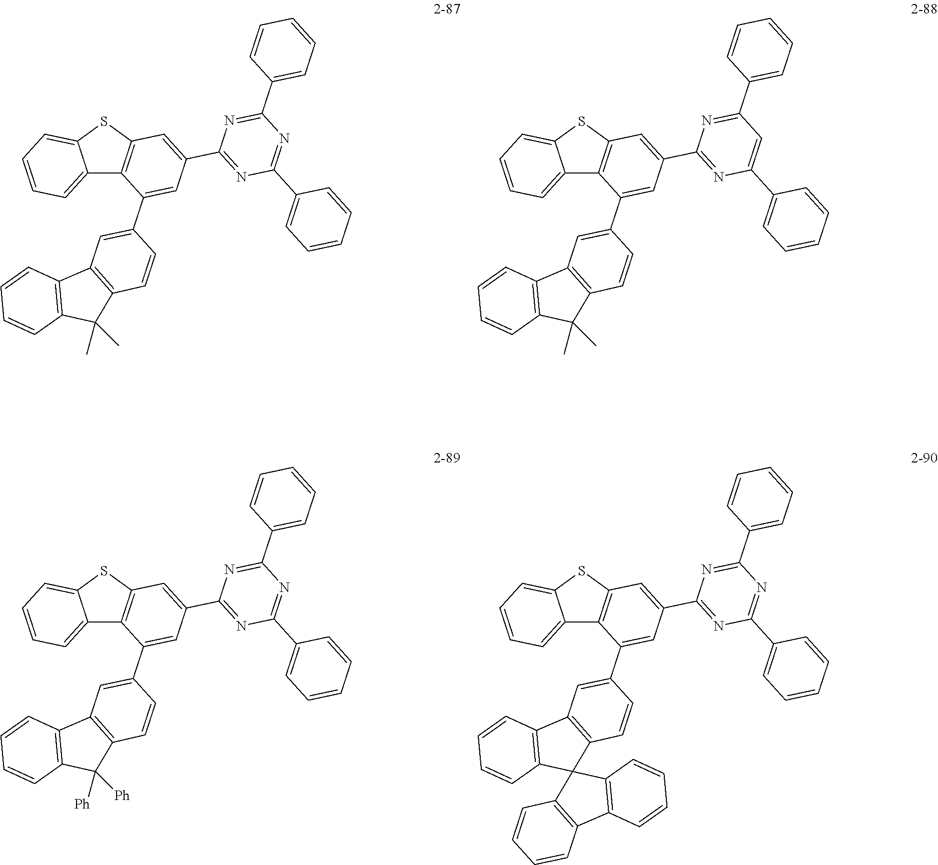 [SMILES: CC1(C)c2ccccc2-c2cc(-c3cc(-c4nc(-c5ccccc5)nc(-c5ccccc5)n4)cc4sc5ccccc5c34)ccc21 ; CC1(C)c2ccccc2-c2cc(-c3cc(-c4nc(-c5ccccc5)cc(-c5ccccc5)n4)cc4sc5ccccc5c34)ccc21 ; c1ccc(-c2nc(-c3ccccc3)nc(-c3cc(-c4ccc5c(c4)-c4ccccc4C5(c4ccccc4)c4ccccc4)c4c(c3)sc3ccccc34)n2)cc1 ; c1ccc(-c2nc(-c3ccccc3)nc(-c3cc(-c4ccc5c(c4)-c4ccccc4C54c5ccccc5-c5ccccc54)c4c(c3)sc3ccccc34)n2)cc1]

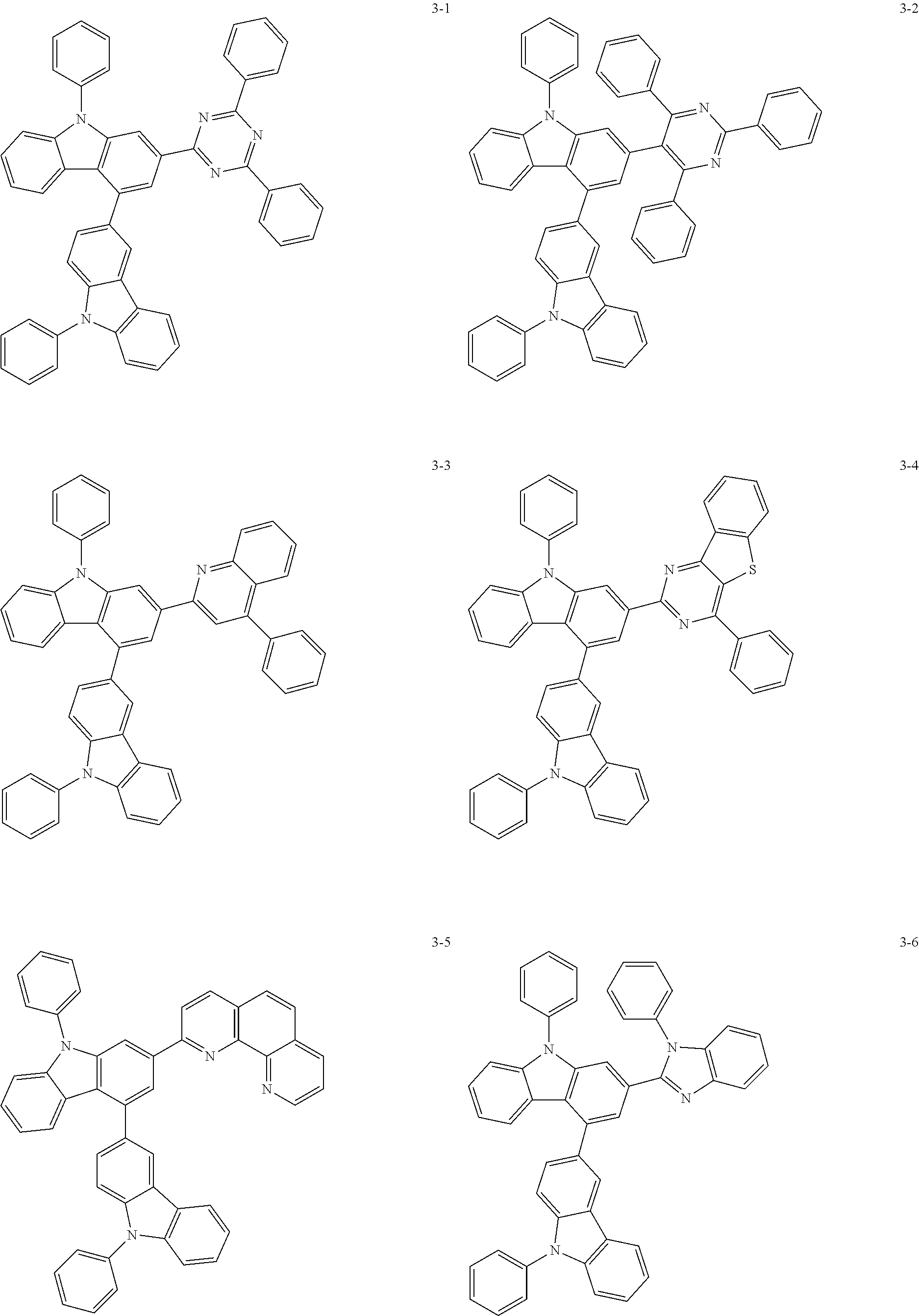

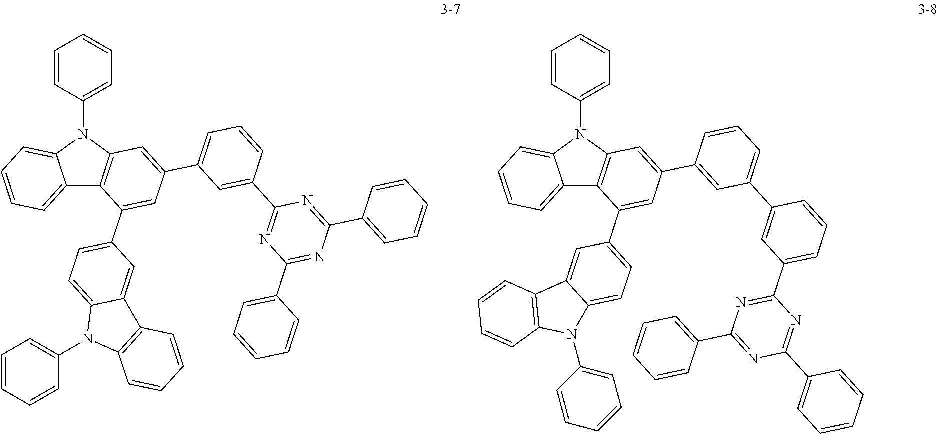 [SMILES: c1ccc(-c2nc(-c3ccccc3)nc(-c3cccc(-c4cc(-c5ccc6c(c5)c5ccccc5n6-c5ccccc5)c5c6ccccc6n(-c6ccccc6)c5c4)c3)n2)cc1 ; c1ccc(-c2nc(-c3ccccc3)nc(-c3cccc(-c4cccc(-c5cc(-c6ccc7c(c6)c6ccccc6n7-c6ccccc6)c6c7ccccc7n(-c7ccccc7)c6c5)c4)c3)n2)cc1]

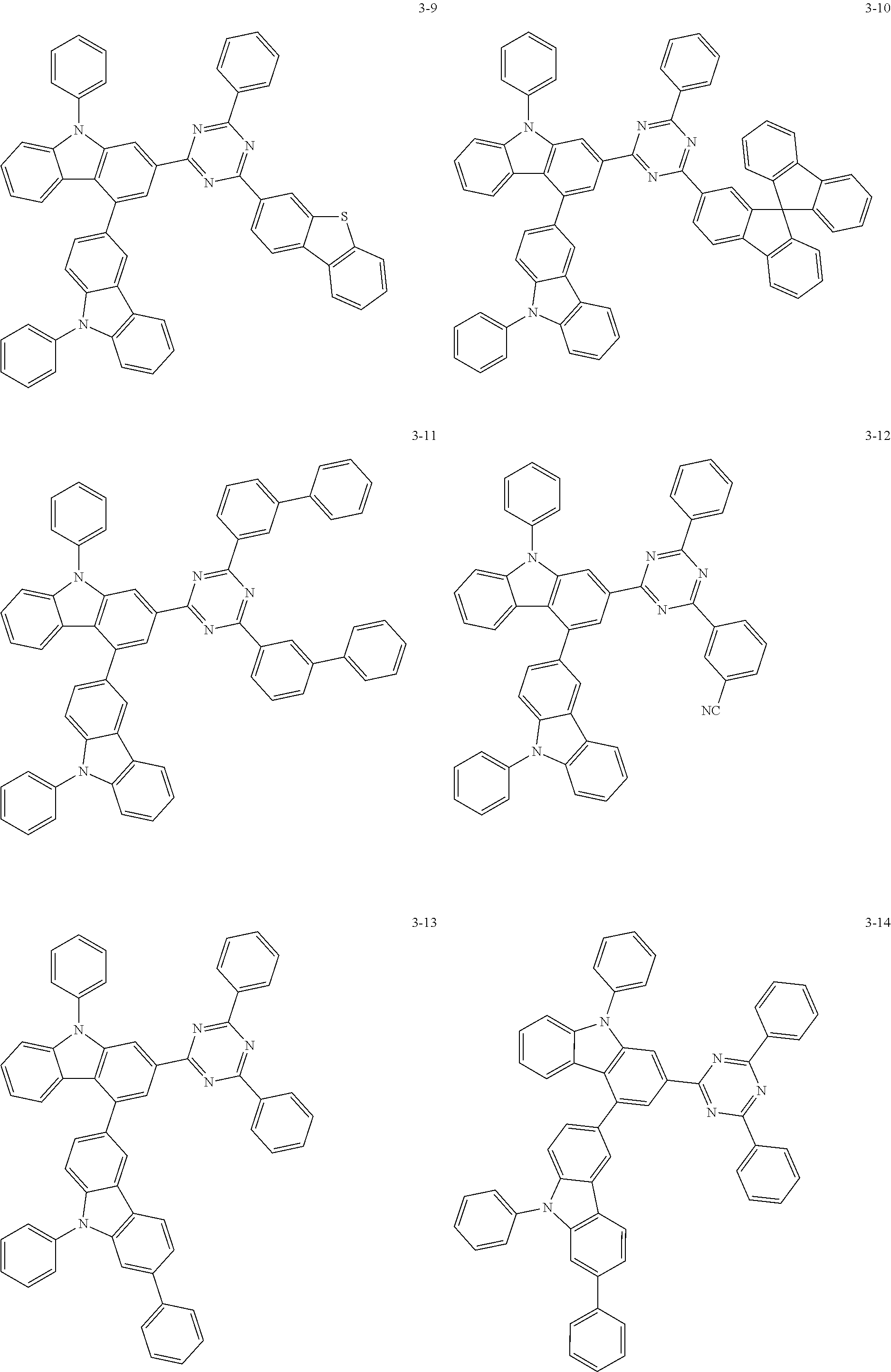

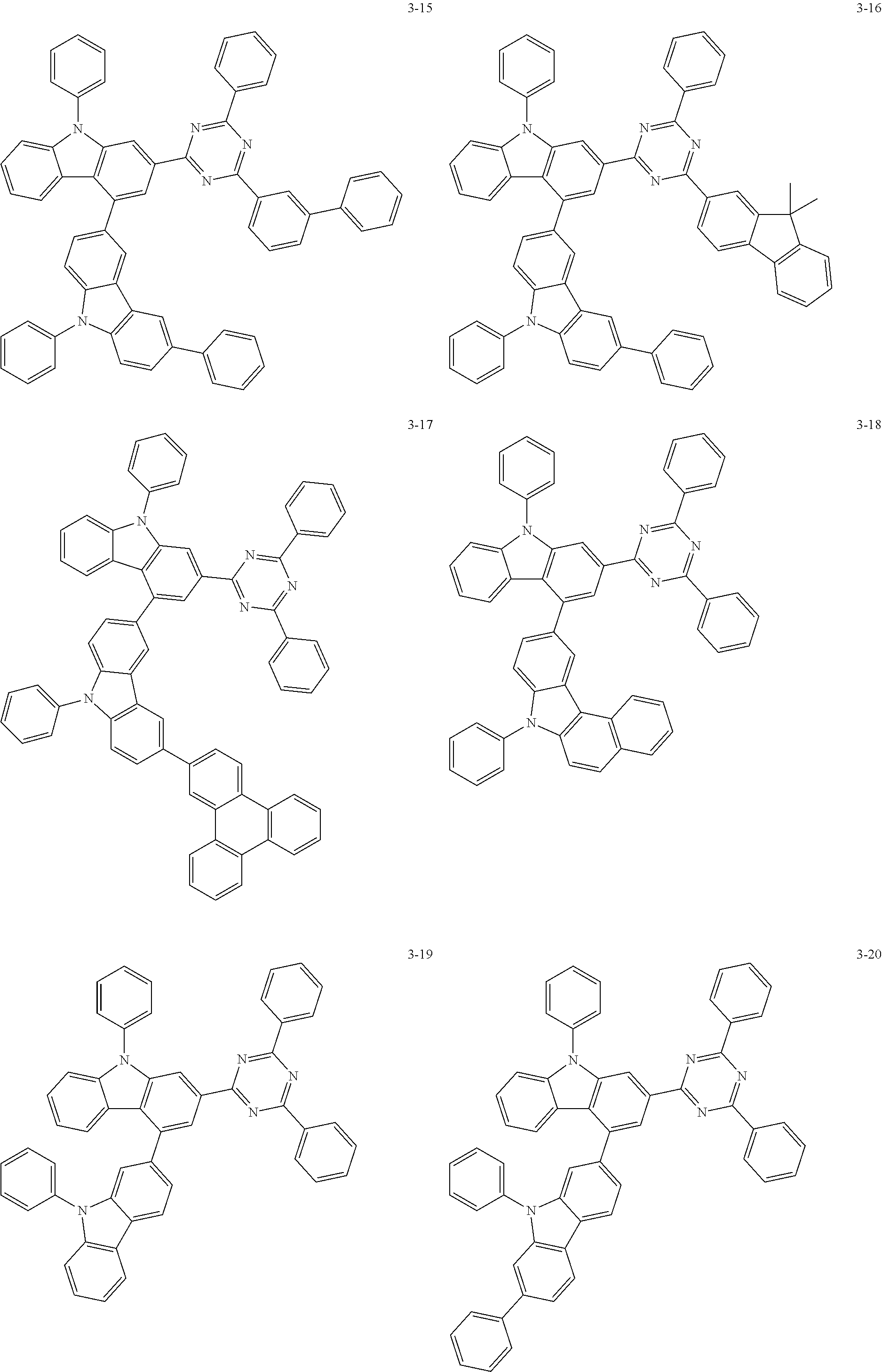

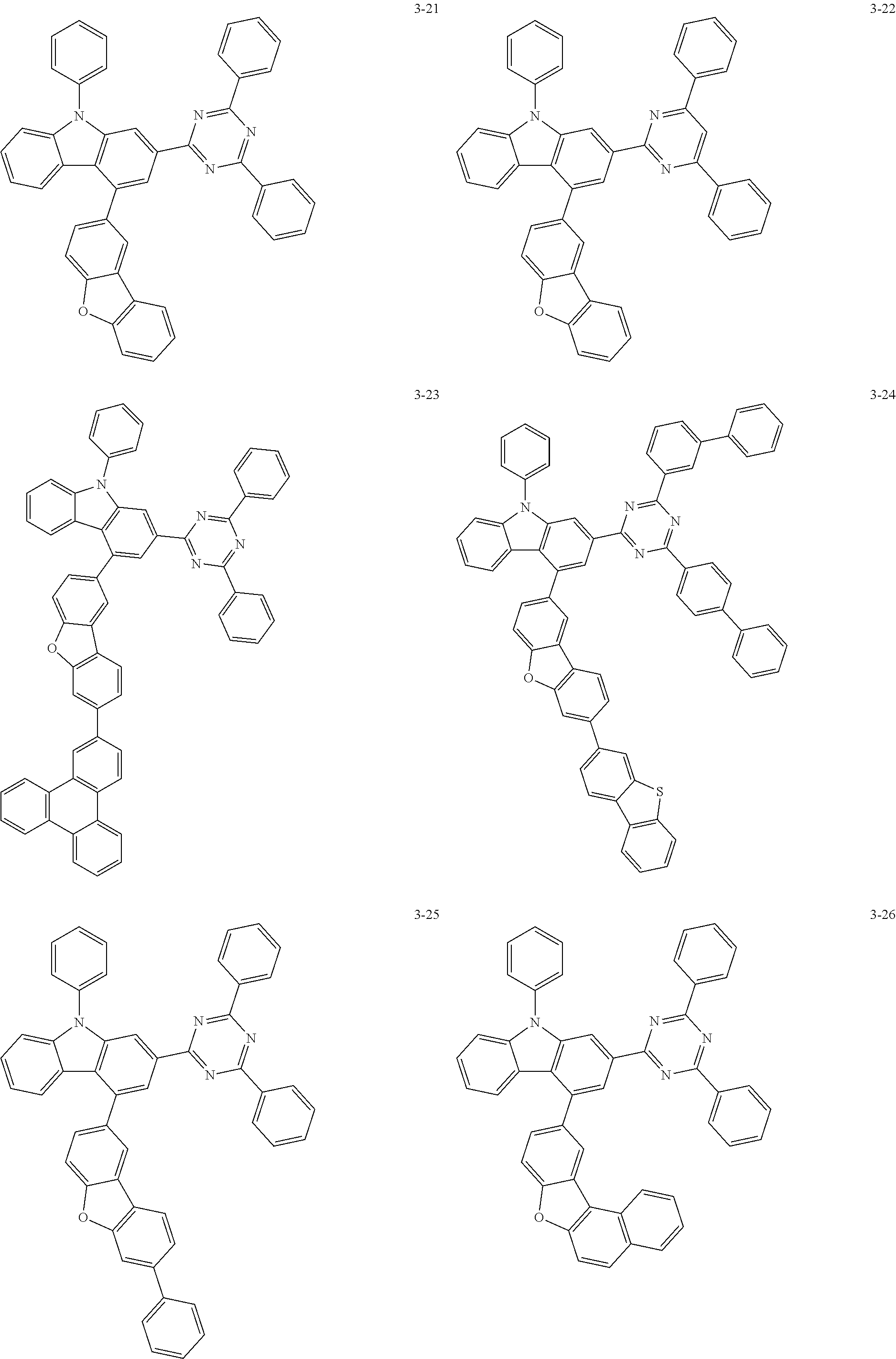

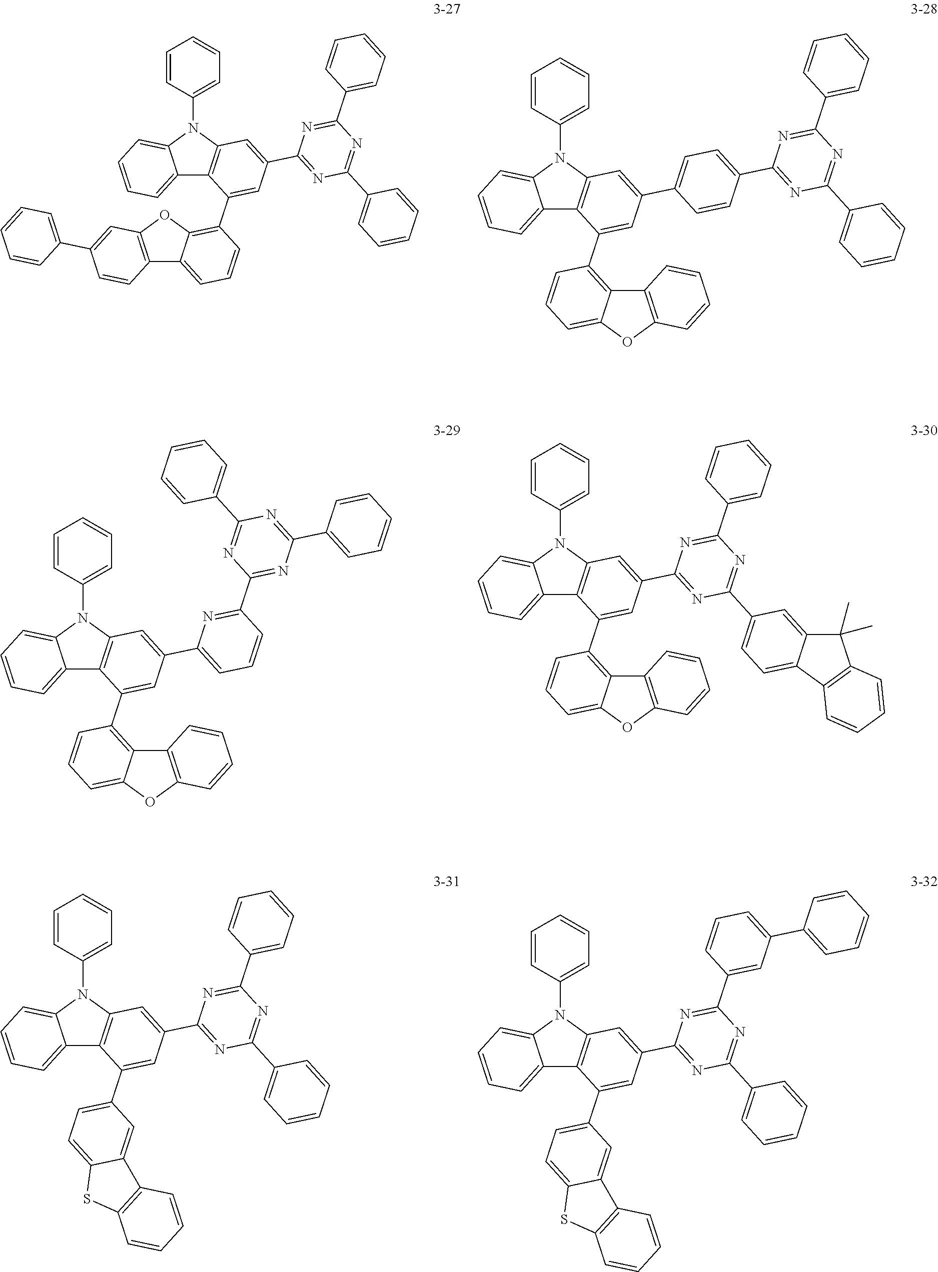

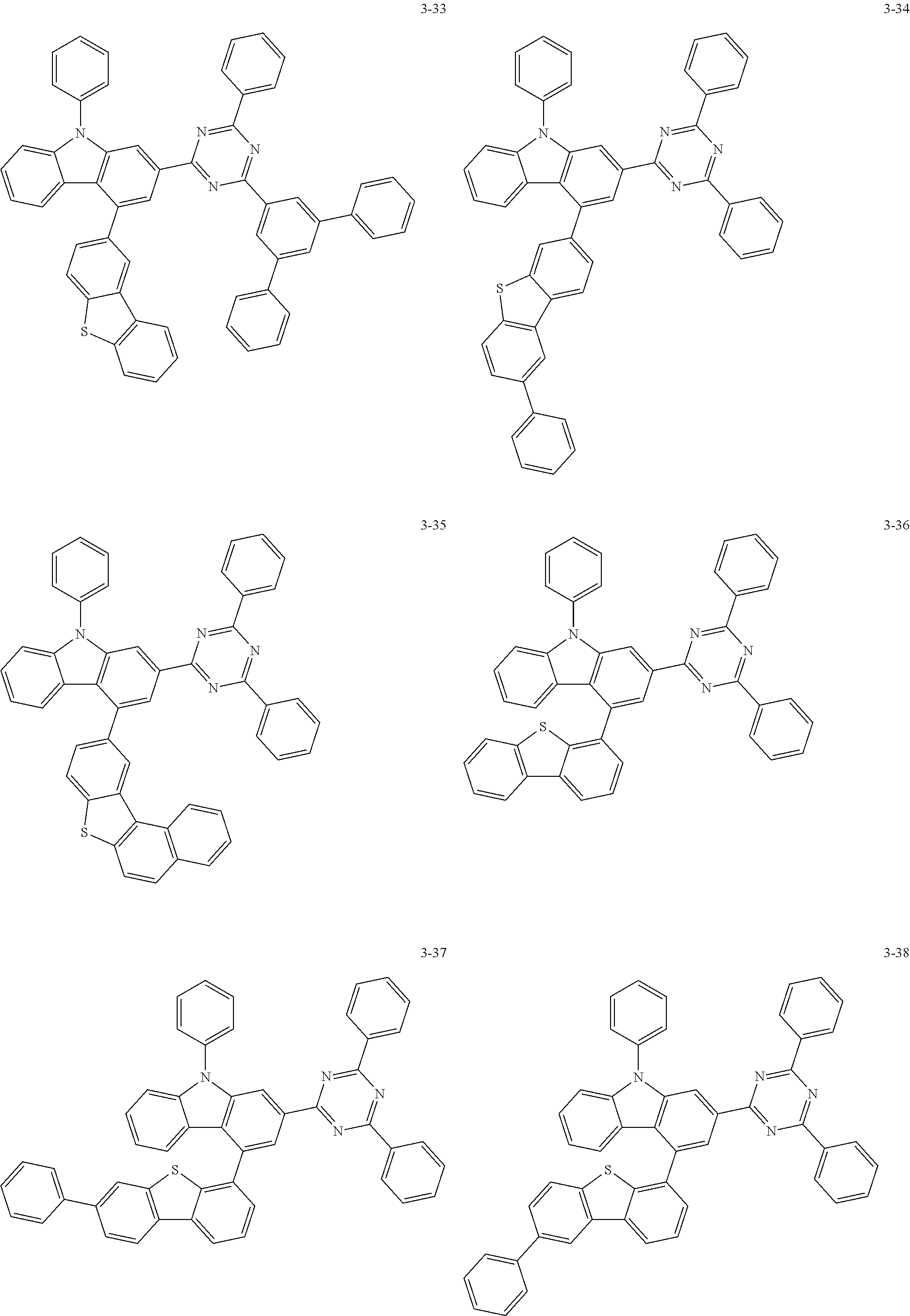

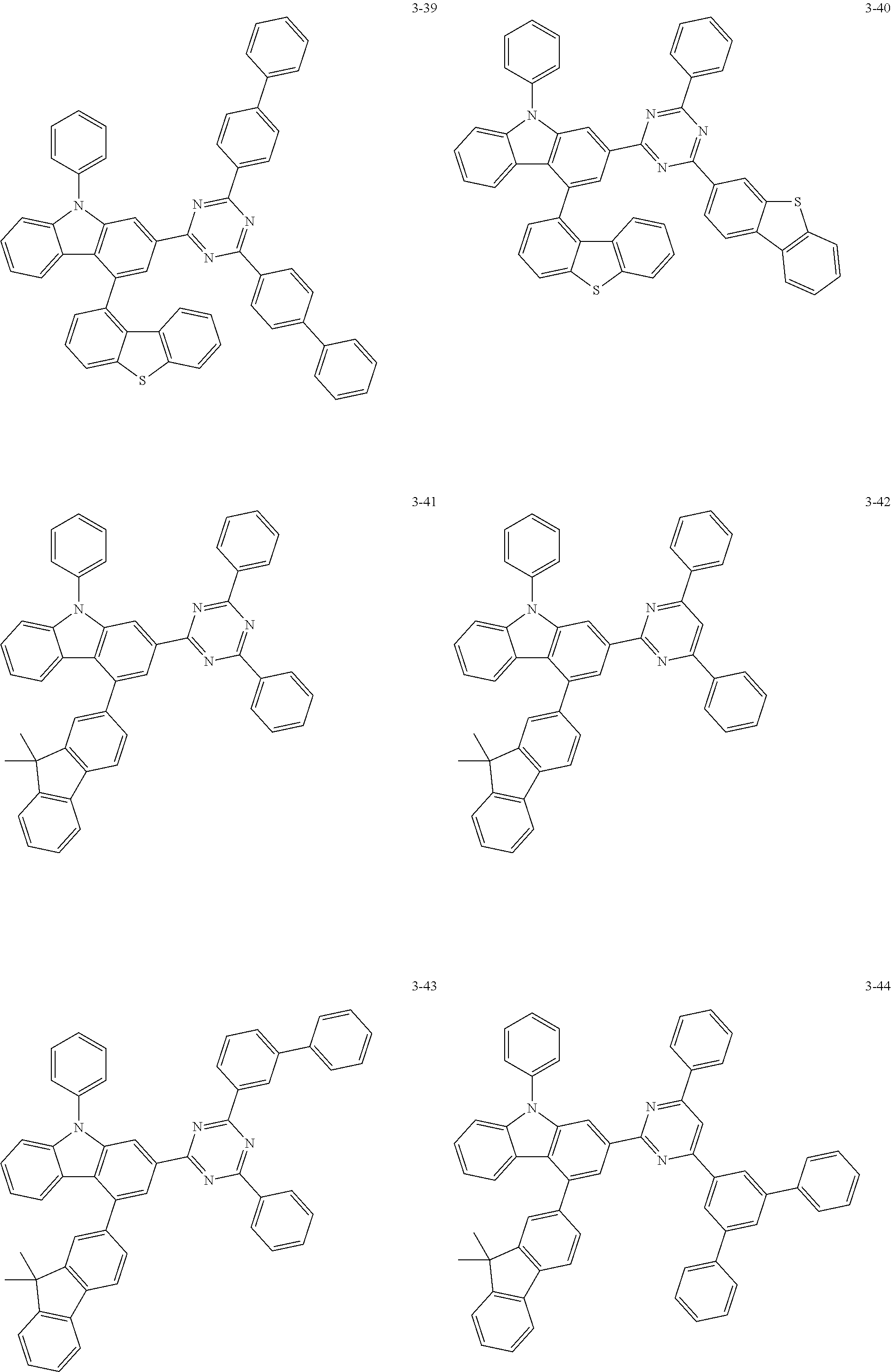

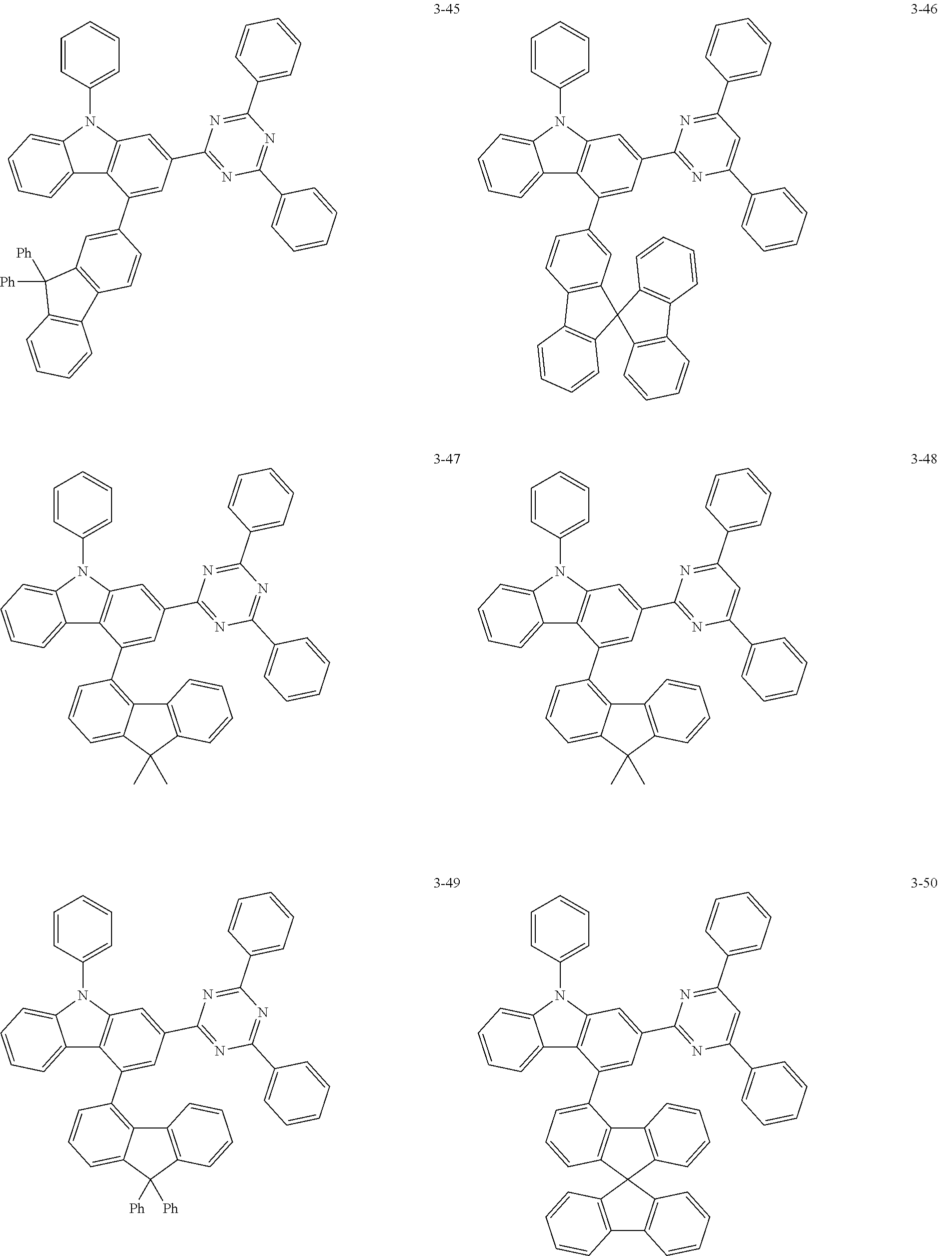

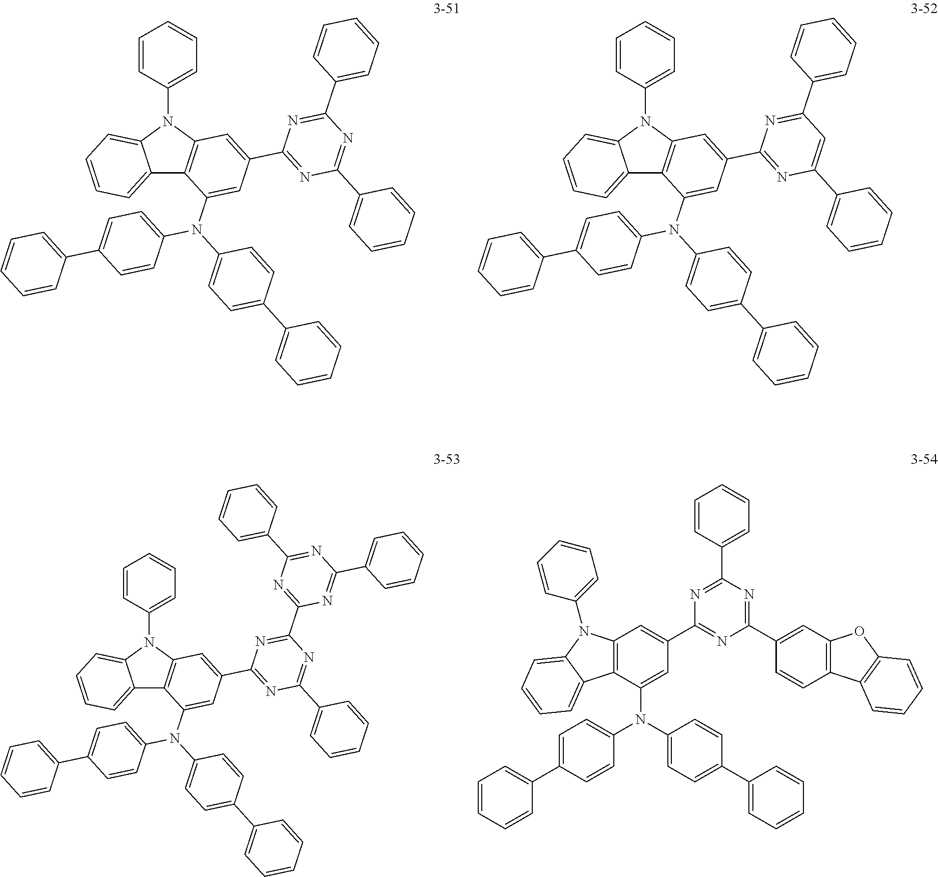 [SMILES: c1ccc(-c2ccc(N(c3ccc(-c4ccccc4)cc3)c3cc(-c4nc(-c5ccccc5)nc(-c5ccccc5)n4)cc4c3c3ccccc3n4-c3ccccc3)cc2)cc1 ; c1ccc(-c2ccc(N(c3ccc(-c4ccccc4)cc3)c3cc(-c4nc(-c5ccccc5)cc(-c5ccccc5)n4)cc4c3c3ccccc3n4-c3ccccc3)cc2)cc1 ; c1ccc(-c2ccc(N(c3ccc(-c4ccccc4)cc3)c3cc(-c4nc(-c5ccccc5)nc(-c5nc(-c6ccccc6)nc(-c6ccccc6)n5)n4)cc4c3c3ccccc3n4-c3ccccc3)cc2)cc1 ; c1ccc(-c2ccc(N(c3ccc(-c4ccccc4)cc3)c3cc(-c4nc(-c5ccccc5)nc(-c5ccc6c(c5)oc5ccccc56)n4)cc4c3c3ccccc3n4-c3ccccc3)cc2)cc1]

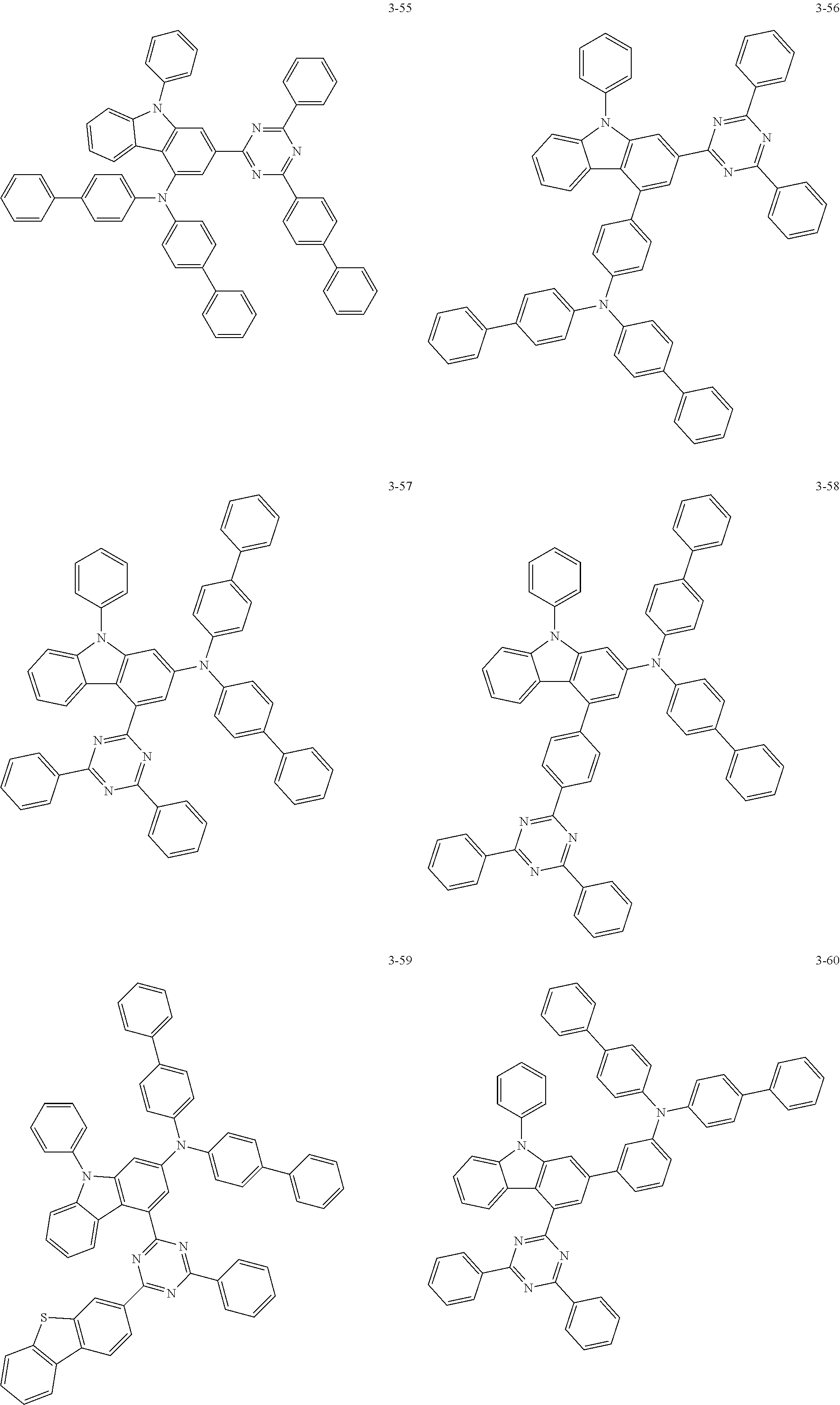

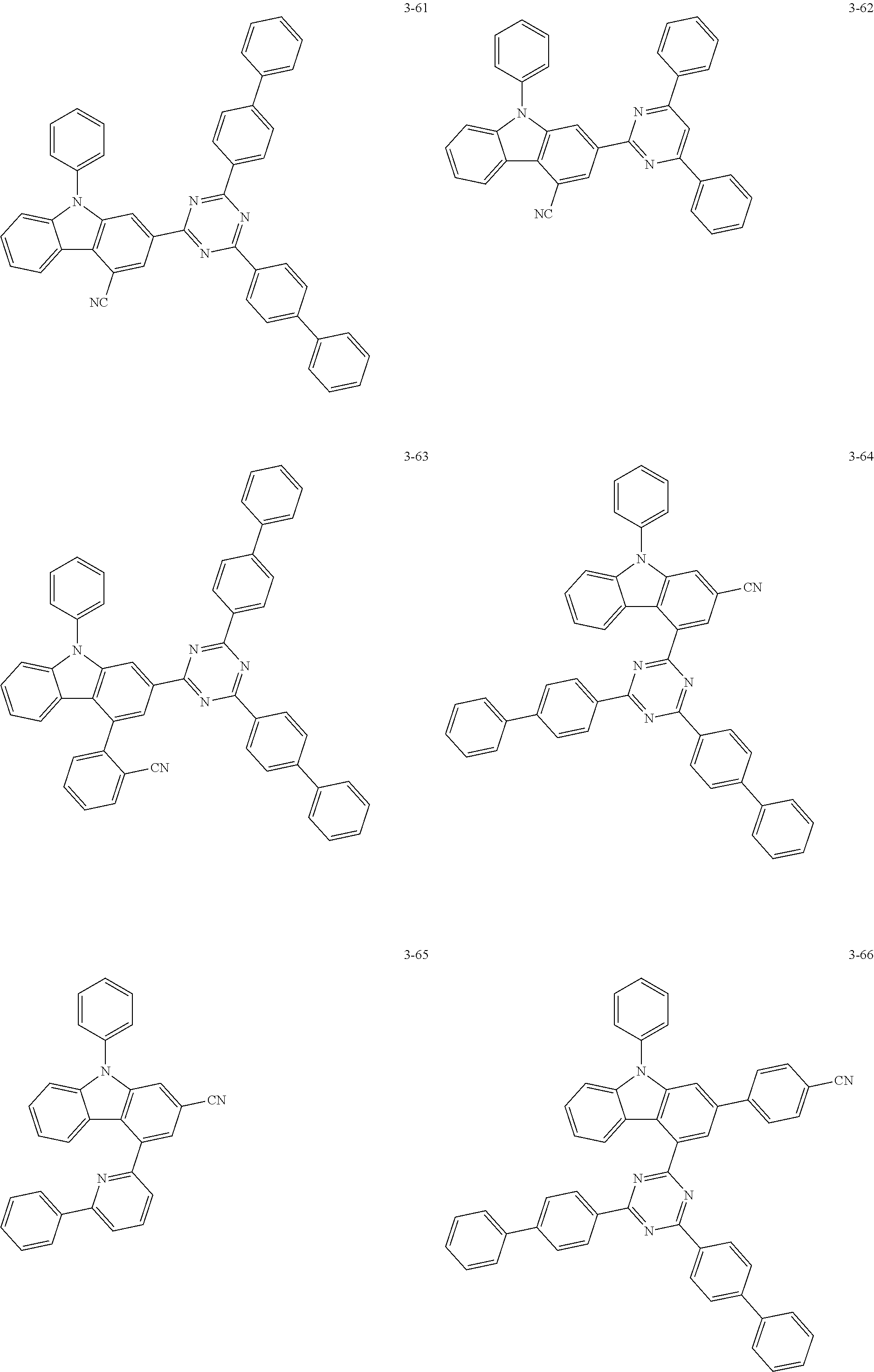

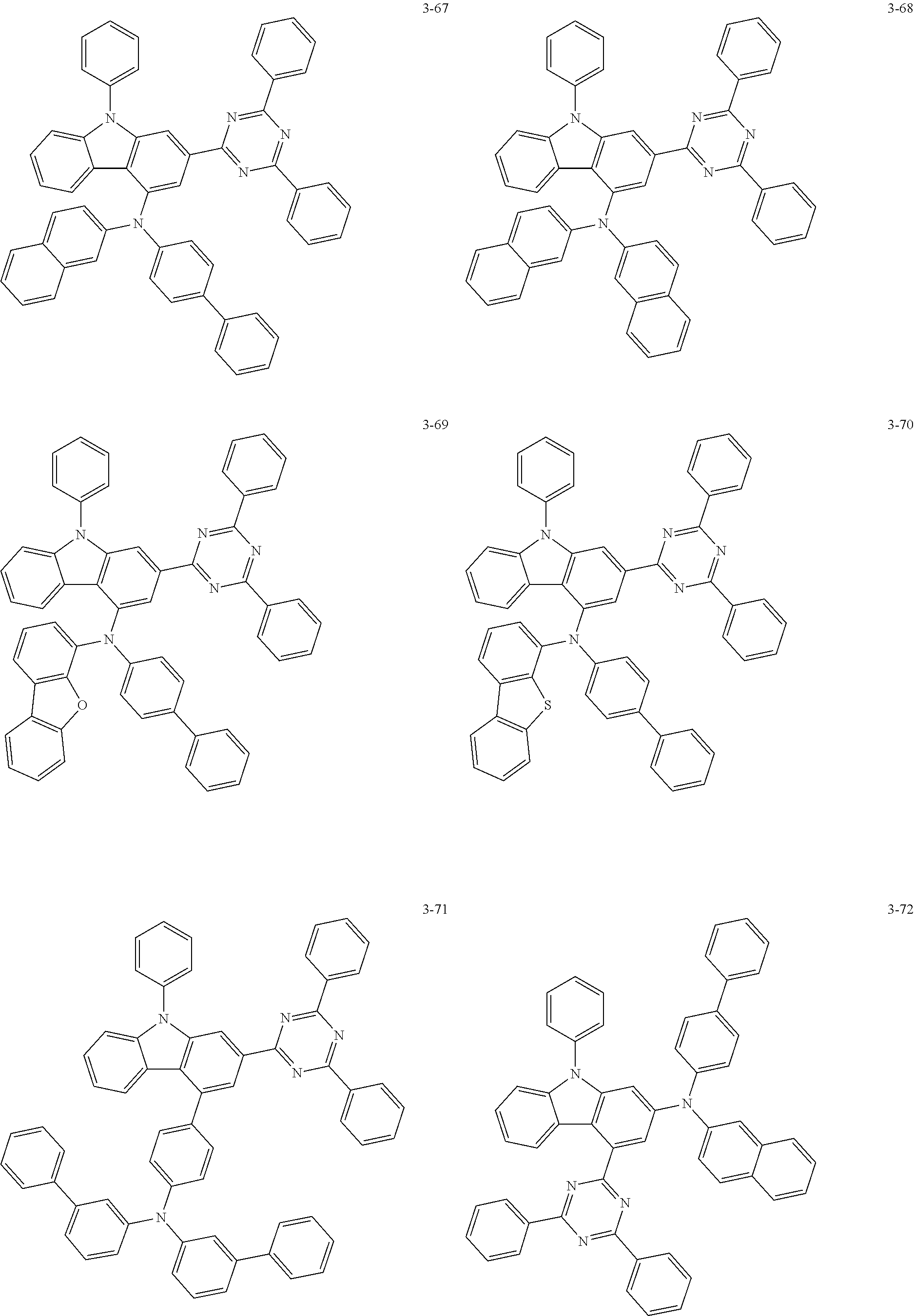

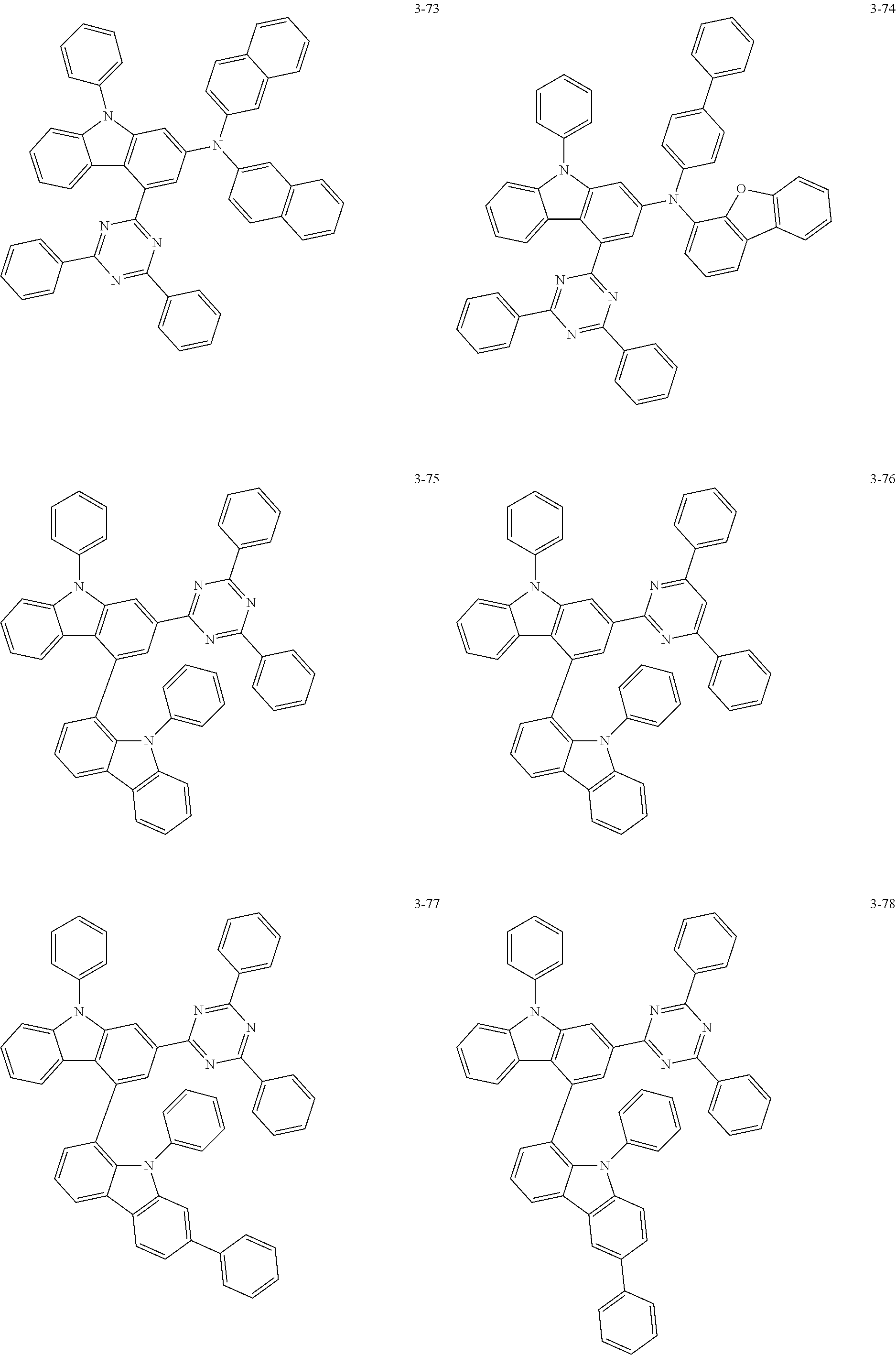

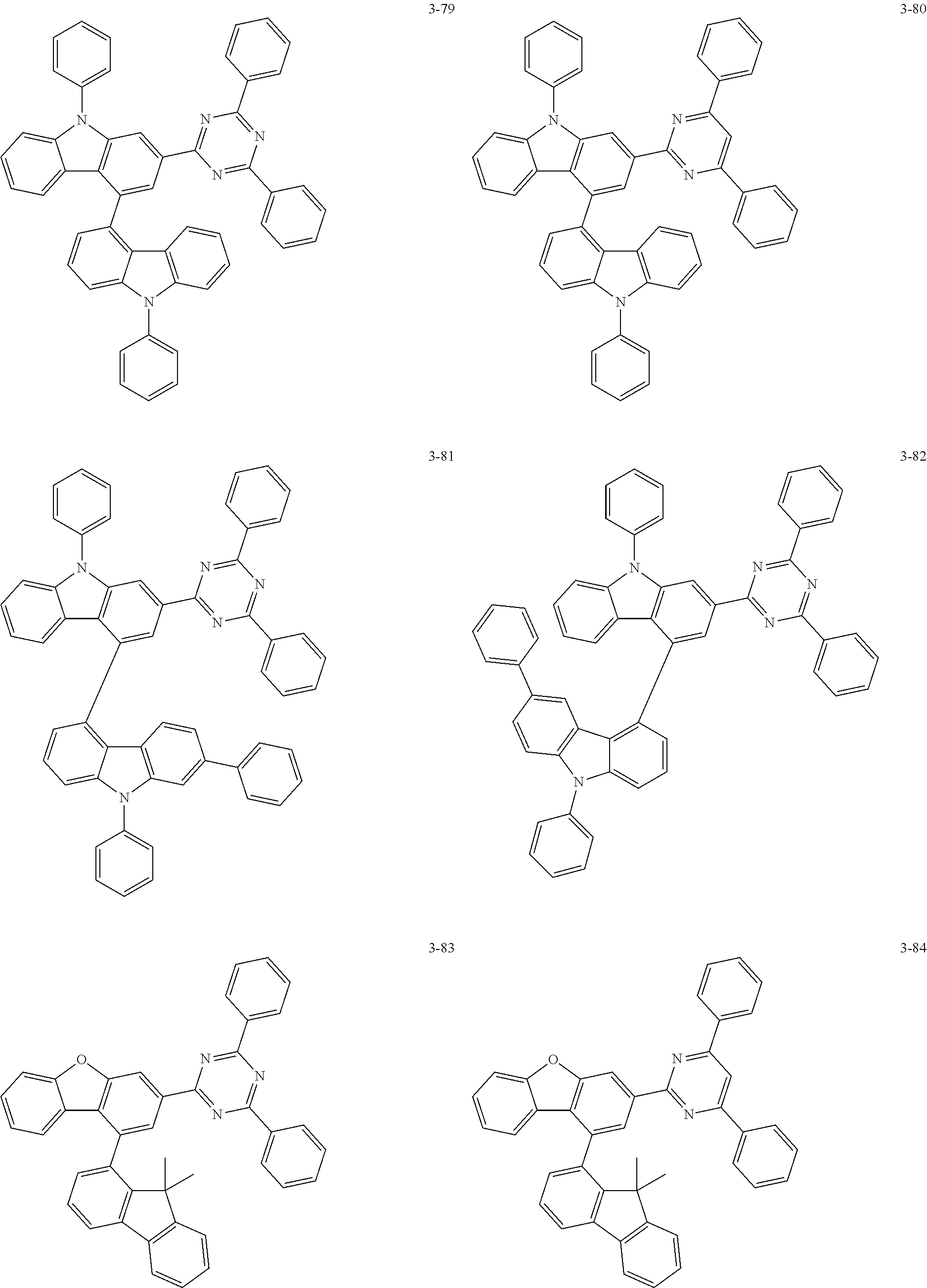

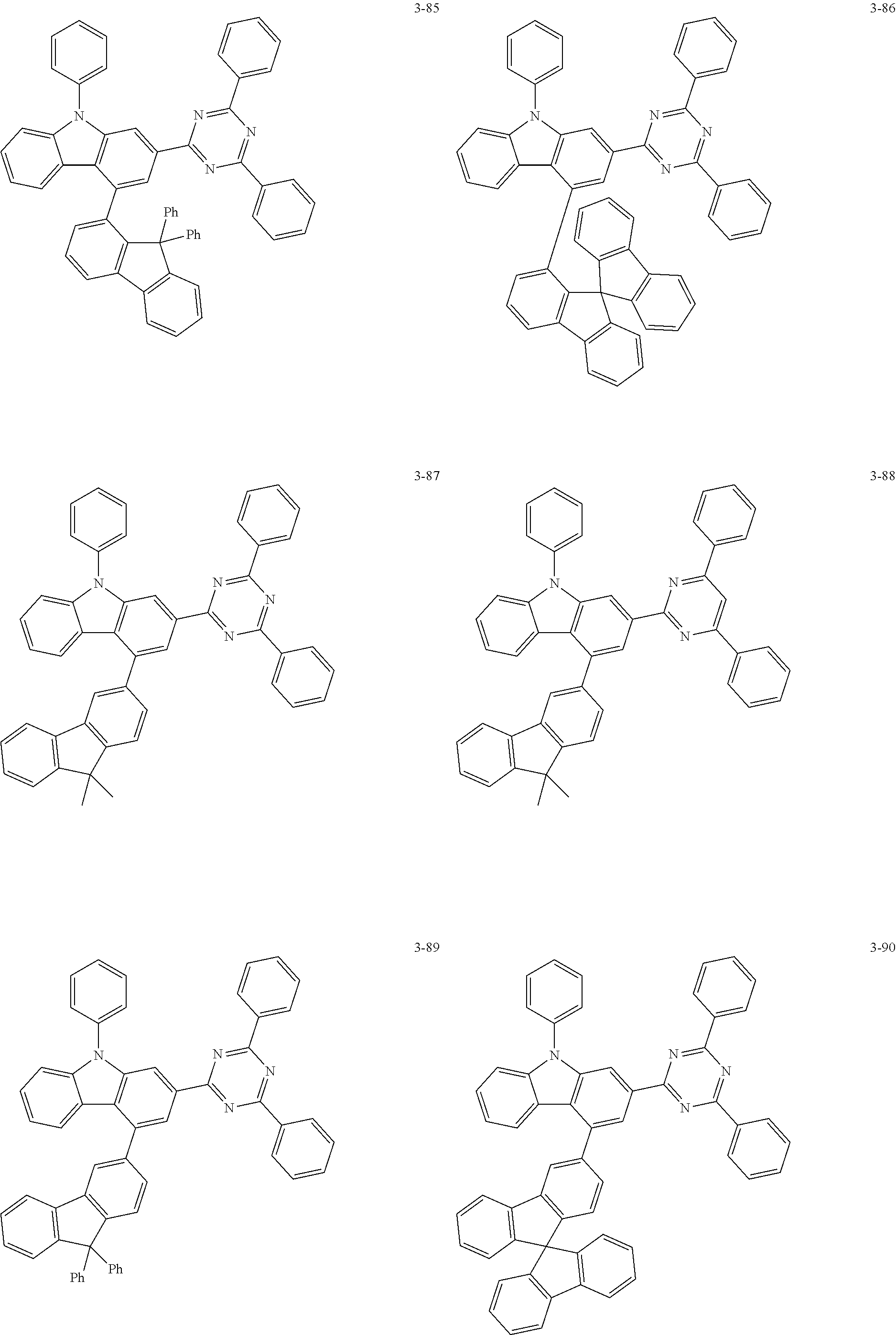

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the compound has a high glass transition temperature (Tg), and has excellent thermal stability. Such an increase in the thermal stability becomes an important factor providing driving stability to a device.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound represented by Chemical Formula 1.

In one embodiment of the present application, the first electrode may be an anode, and the second electrode may be a cathode.

In another embodiment, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the compound represented by Chemical Formula 1 may be used as a material of the green organic light emitting device.

In one embodiment of the present application, the organic light emitting device may be a red organic light emitting device, and the compound represented by Chemical Formula 1 may be used as a material of the red organic light emitting device.

In one embodiment of the present application, the organic light emitting device may be a blue organic light emitting device, and the heterocyclic compound according to Chemical Formula 1 may be used as a light emitting layer material of the blue organic light emitting device.

In one embodiment of the present application, the organic light emitting device may be a green organic light emitting device, and the compound represented by Chemical Formula 1 may be used as a light emitting layer material of the green organic light emitting device.

In one embodiment of the present application, the organic light emitting device may be a red organic light emitting device, and the compound represented by Chemical Formula 1 may be used as a light emitting layer material of the red organic light emitting device.

Specific details on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, or may also be formed in a multilayer structure in which two or more organic material layers are laminated. For example, the organic light emitting device according to one embodiment of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise a smaller number of organic material layers.

In the organic light emitting device according to one embodiment of the present application, the organic material layer comprising the heterocyclic compound represented by Chemical Formula 1 further comprises a heterocyclic compound represented by the following Chemical Formula 2.

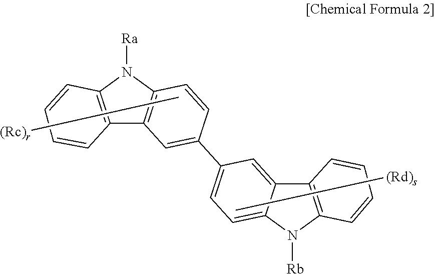

In Chemical Formula 2,

Rc and Rd are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiR₁₀R₁₁R₁₂; —P(═O) R₁₀R₁₁; and an amine group unsubstituted or substituted with a substituted or unsubstituted alkyl group, a substituted or unsubstituted aryl group or a substituted or unsubstituted heteroaryl group, or two or more groups adjacent to each other bond to each other to form a substituted or unsubstituted aromatic hydrocarbon ring or a substituted or unsubstituted heteroring,

R₁₀, R₁₁ and R₁₂ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group, Ra and Rb are the same as or different from each other, and each independently a substituted or unsubstituted aryl group; or a substituted or unsubstituted heteroaryl group, and r and s are an integer of 0 to 7.

In the organic light emitting device according to one embodiment of the present application, Rc and Rd of Chemical Formula 2 may be hydrogen.

In the organic light emitting device according to one embodiment of the present application, Ra and Rb of Chemical Formula 2 are the same as or different from each other, and may be each independently a substituted or unsubstituted aryl group; or a substituted or unsubstituted heteroaryl group.

In the organic light emitting device according to another embodiment, Ra and Rb of Chemical Formula 2 are the same as or different from each other, and may be each independently a substituted or unsubstituted C6 to C40 aryl group; or a substituted or unsubstituted C6 to C40 heteroaryl group.

In the organic light emitting device according to another embodiment, Ra and Rb of Chemical Formula 2 are the same as or different from each other, and may be each independently a C6 to C40 aryl group unsubstituted or substituted with one or more substituents selected from the group consisting of a C1 to C40 alkyl group, a C6 to C40 aryl group, —CN and —SiR₁₀R₁₁R₁₂; or a C2 to C40 heteroaryl group unsubstituted or substituted with one or more substituents selected from the group consisting of a C6 to C40 aryl group and a C2 to C40 heteroaryl group.

In the organic light emitting device according to another embodiment, Ra and Rb of Chemical Formula 2 are the same as or different from each other, and may be each independently a C6 to C40 aryl group unsubstituted or substituted with one or more substituents selected from the group consisting of a C1 to C40 alkyl group, a C6 to C40 aryl group, —CN and —SiR₁₀R₁₁R₁₂.

In the organic light emitting device according to another embodiment, Ra and Rb of Chemical Formula 2 are the same as or different from each other, and may be each independently a phenyl group unsubstituted or substituted with a phenyl group, —CN or —SiR₁₀R₁₁R₁₂; a biphenyl group unsubstituted or substituted with a phenyl group; a naphthyl group; a fluorenyl group unsubstituted or substituted with a methyl group or a phenyl group; a spirobifluorenyl group; or a triphenylenyl group.

In the organic light emitting device according to one embodiment of the present application, R₁₀, R₁₁ and R₁₂ of Chemical Formula 2 may be a phenyl group.

When comprising the compound of Chemical Formula 1 and the compound of Chemical Formula 2 in an organic material layer of an organic light emitting device, more superior efficiency and lifetime effects are obtained. Such results may lead to a forecast that an exciplex phenomenon occurs when comprising the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may decrease, which resultantly helps with enhancement in the lifetime.

In one embodiment of the present application, the heterocyclic compound represented by Chemical Formula 2 may be any one selected from among the following compounds.

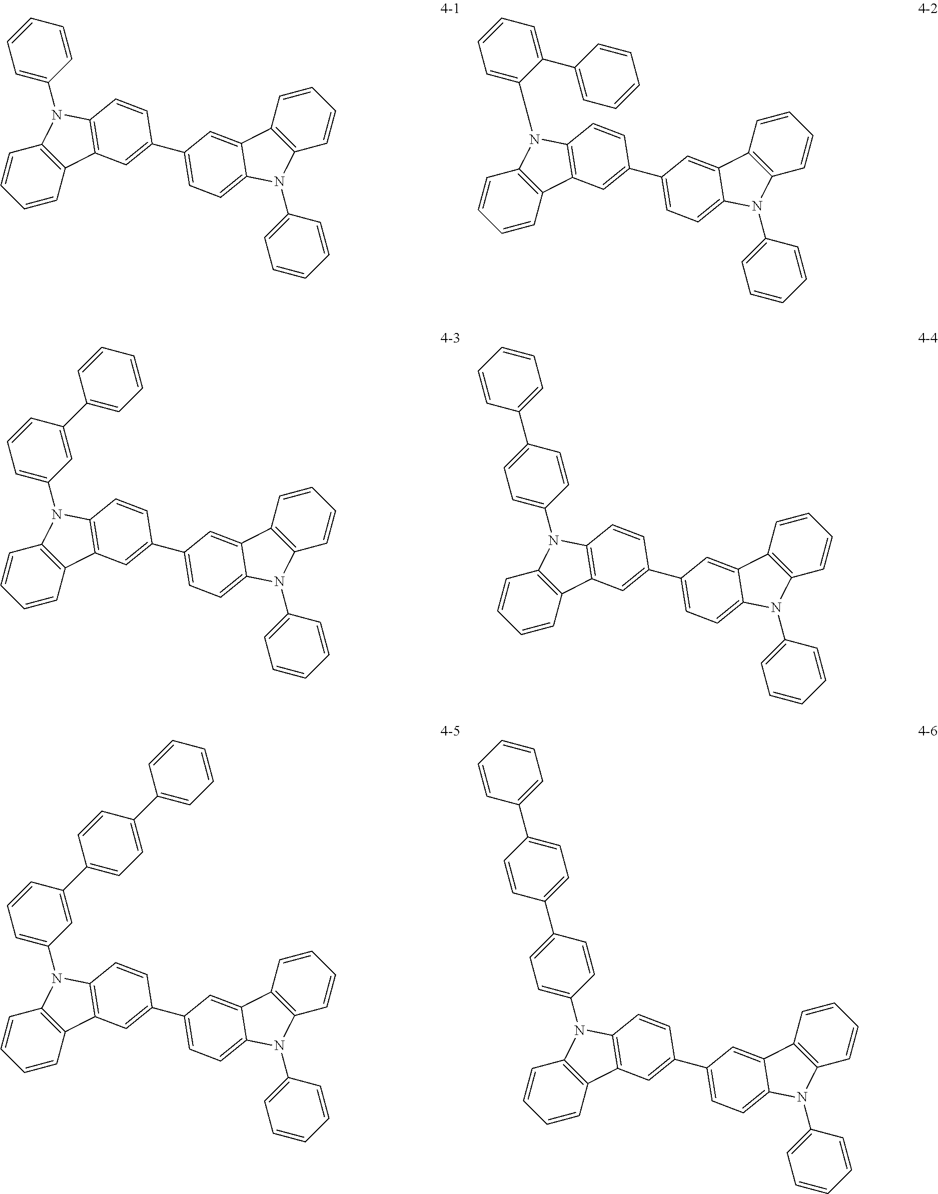 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

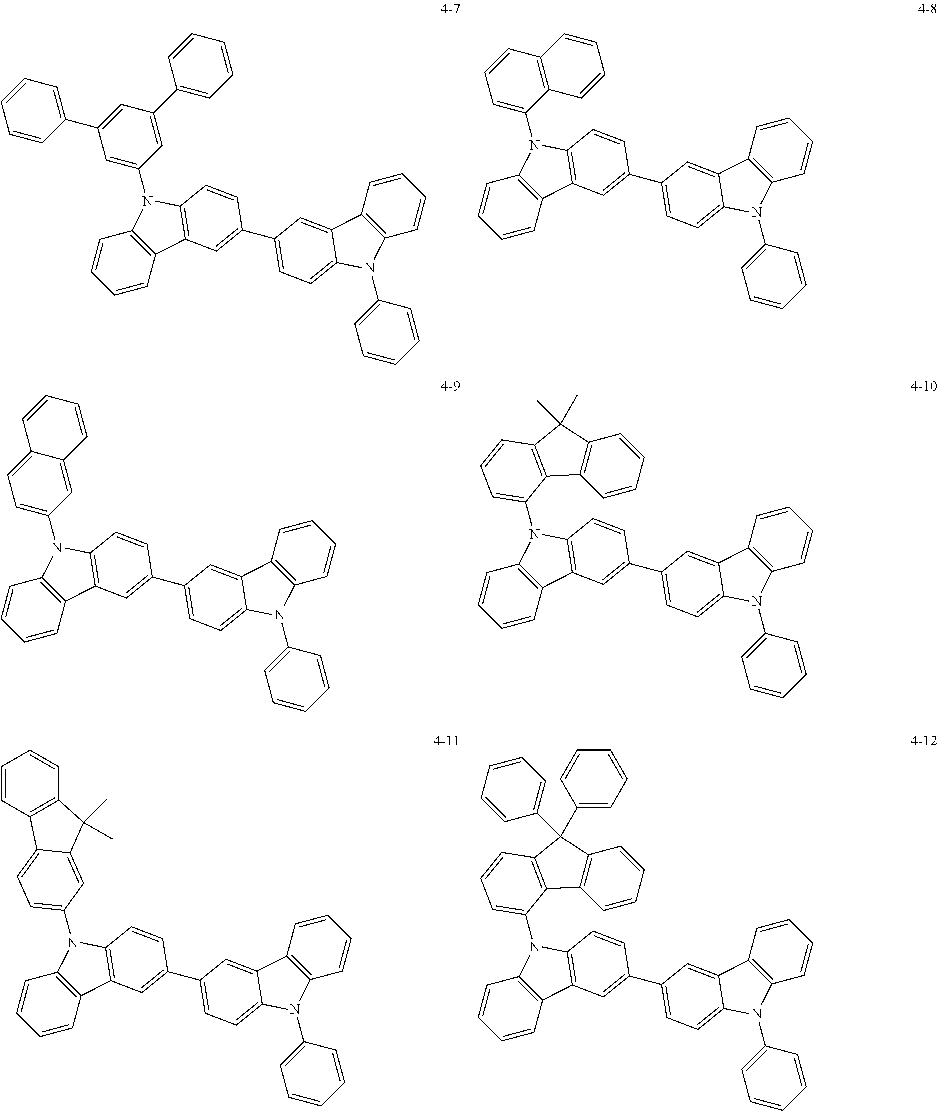 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

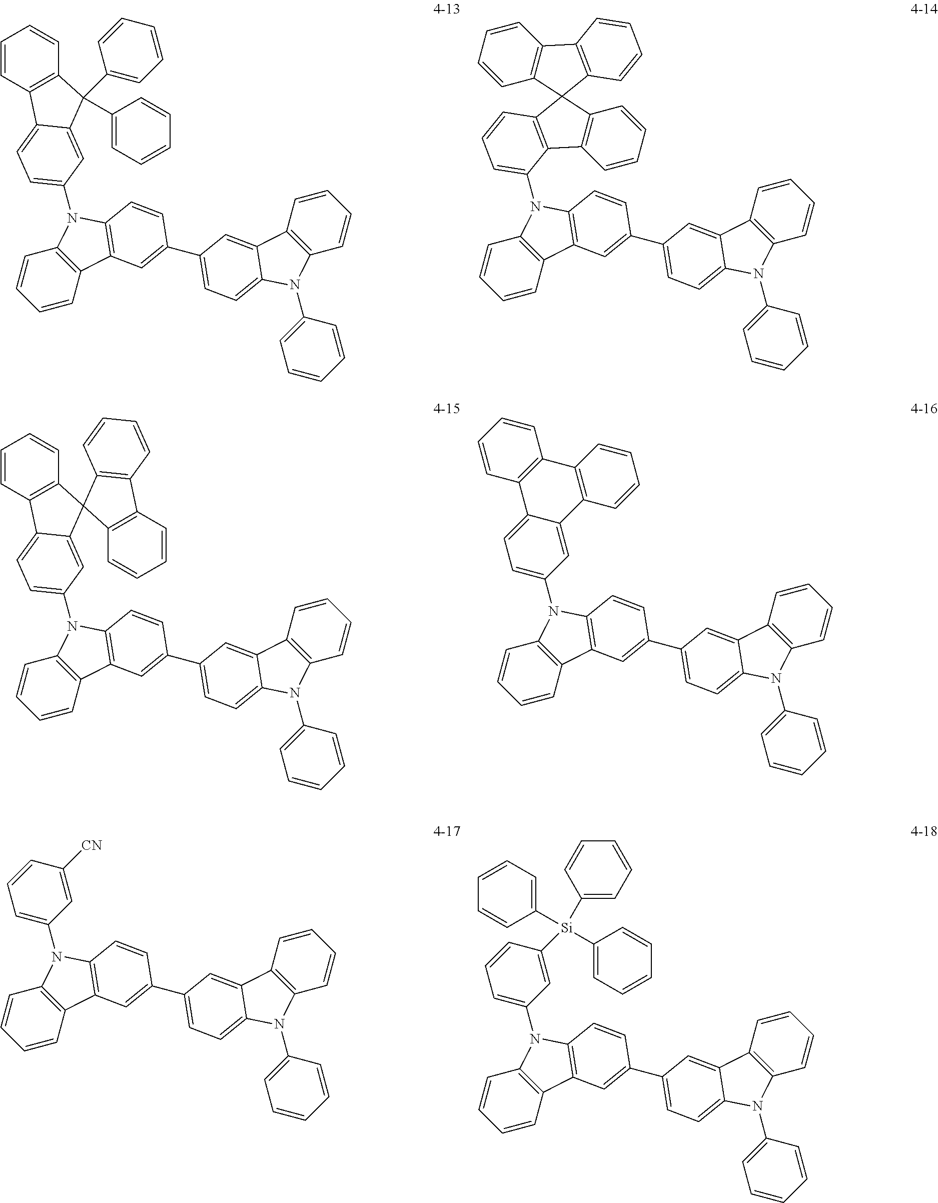

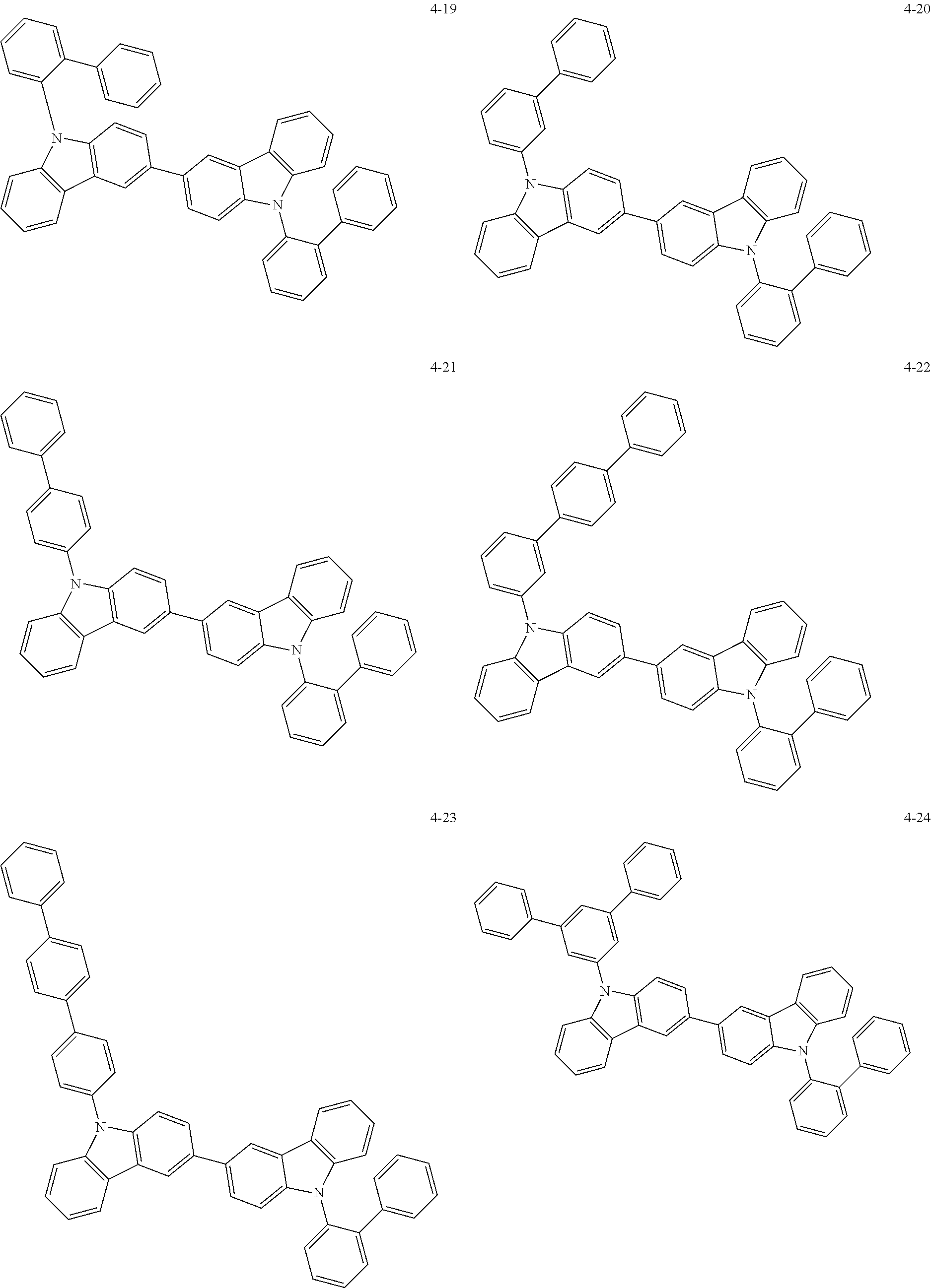

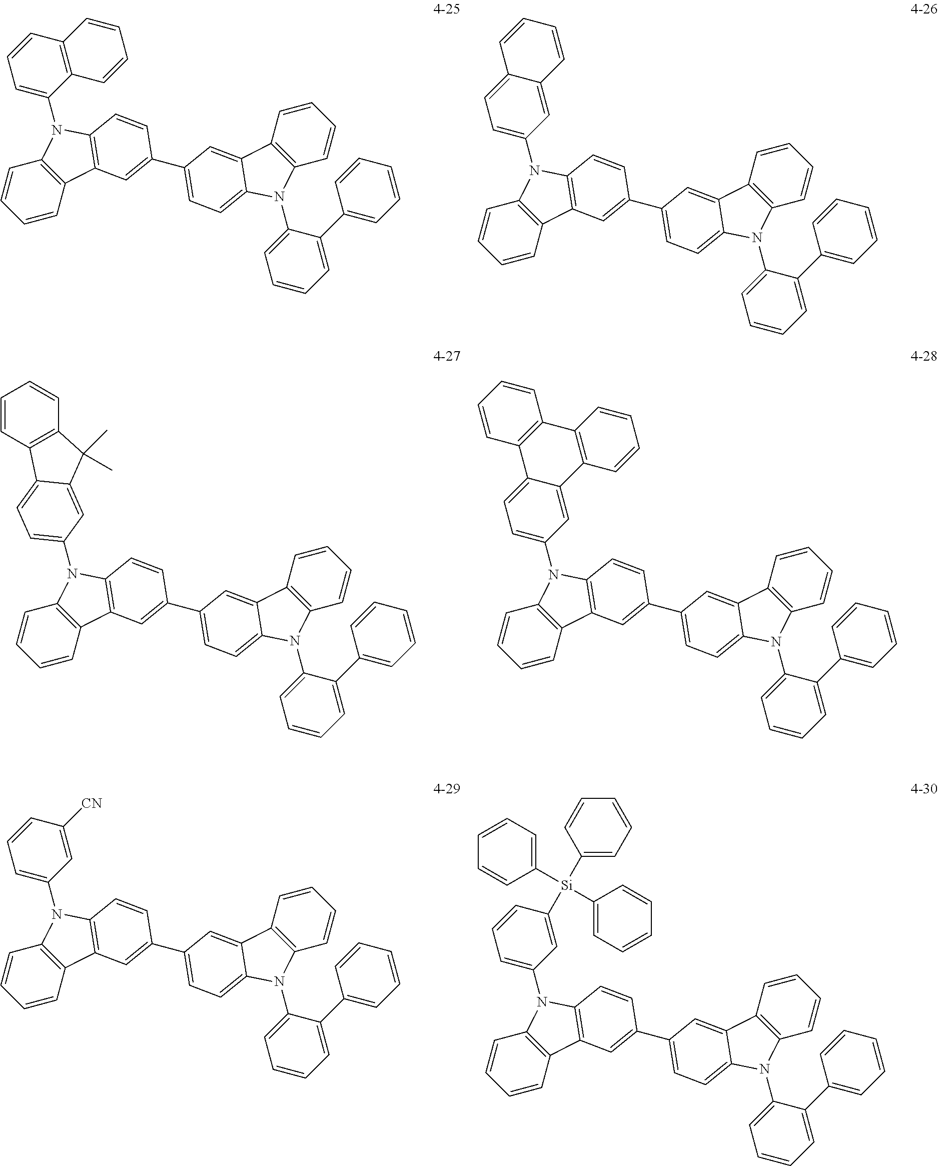

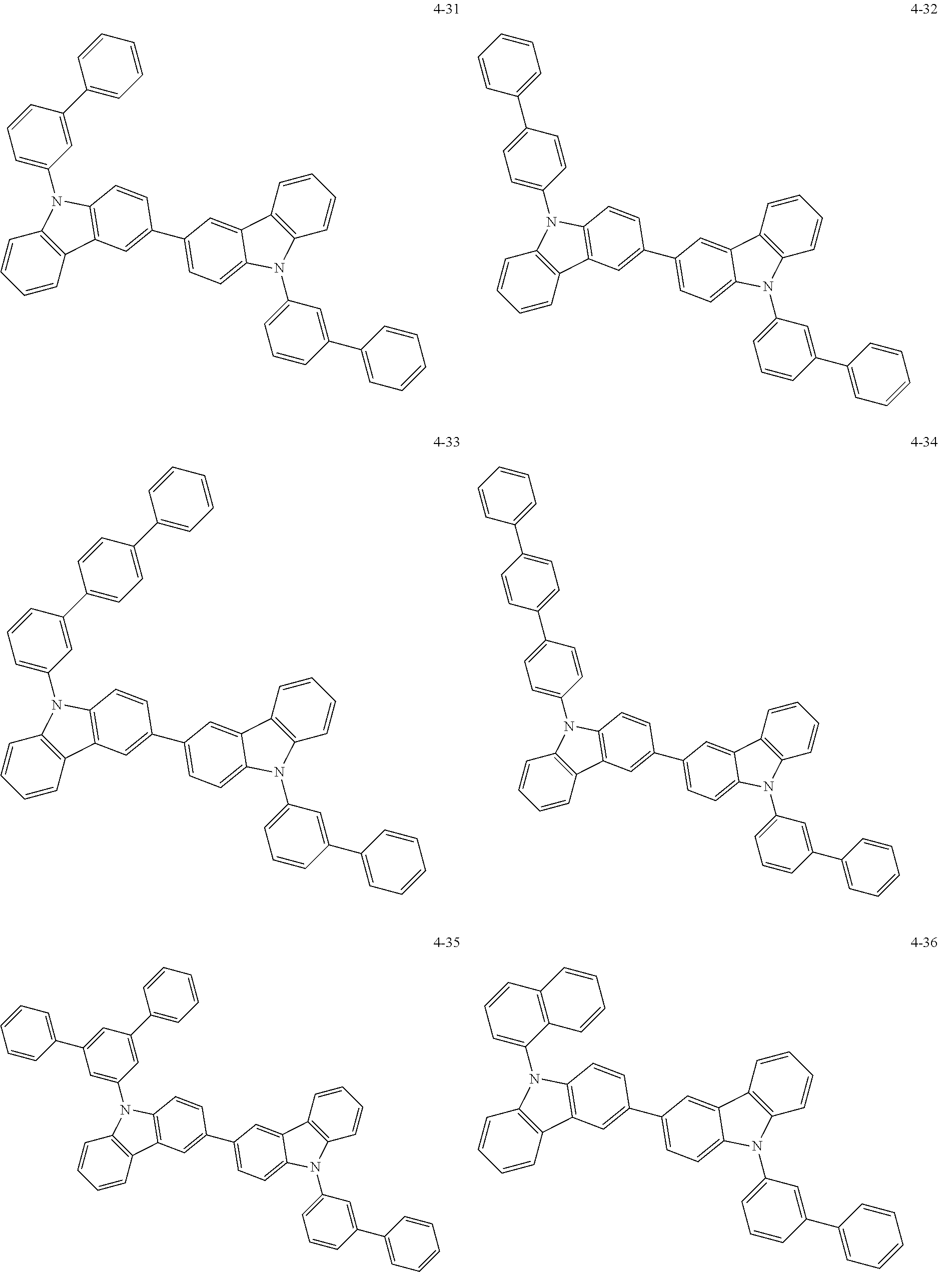

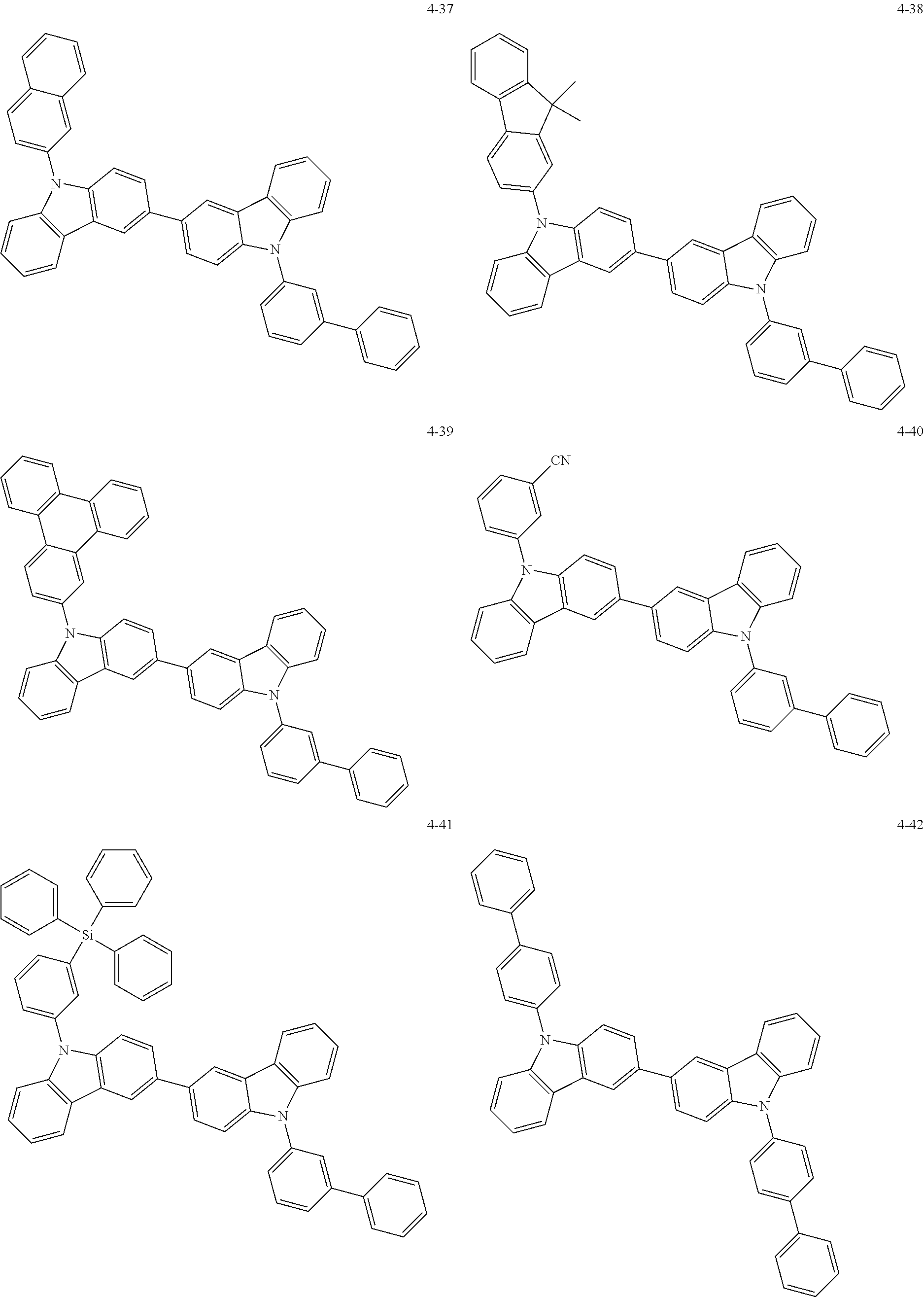

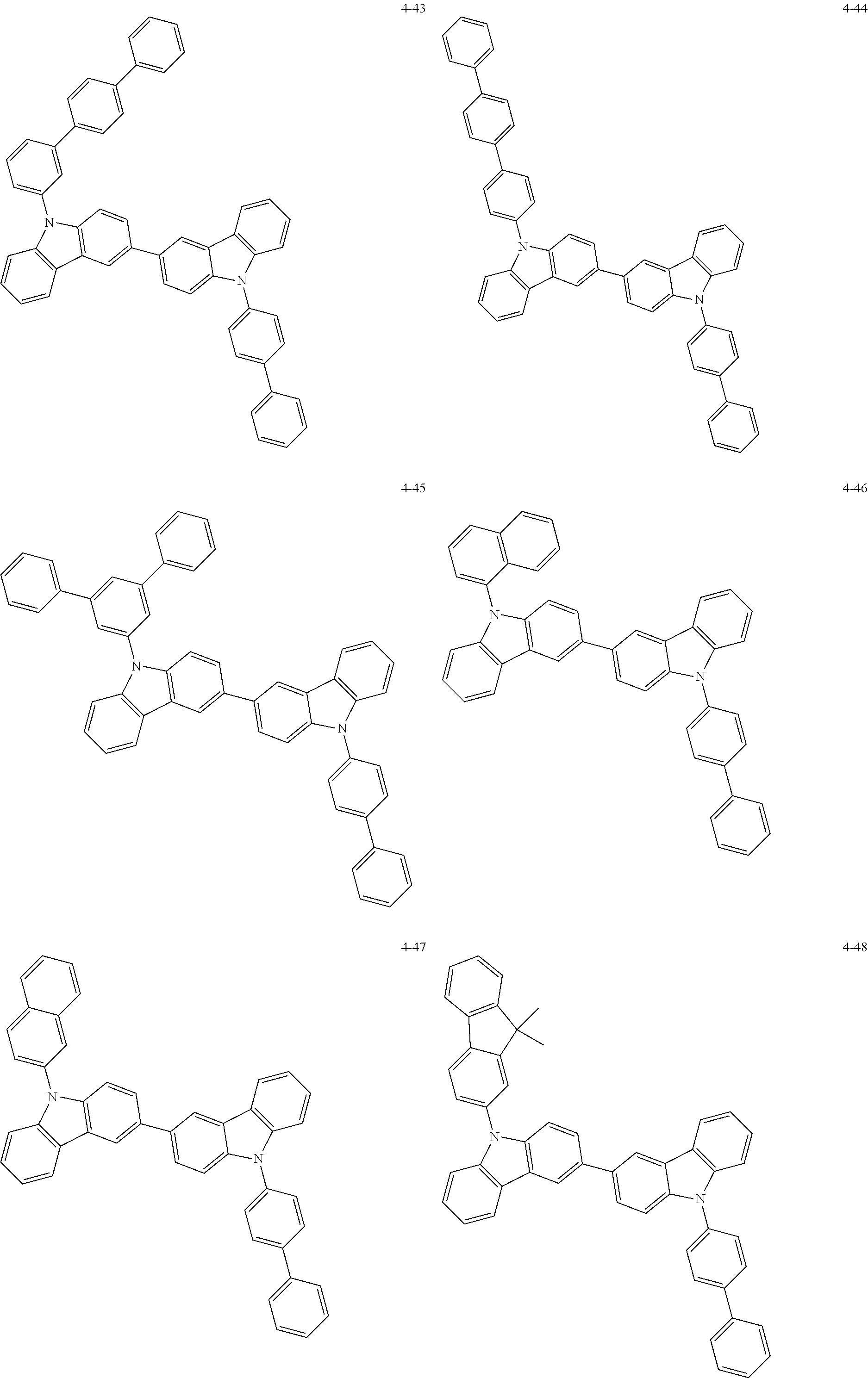

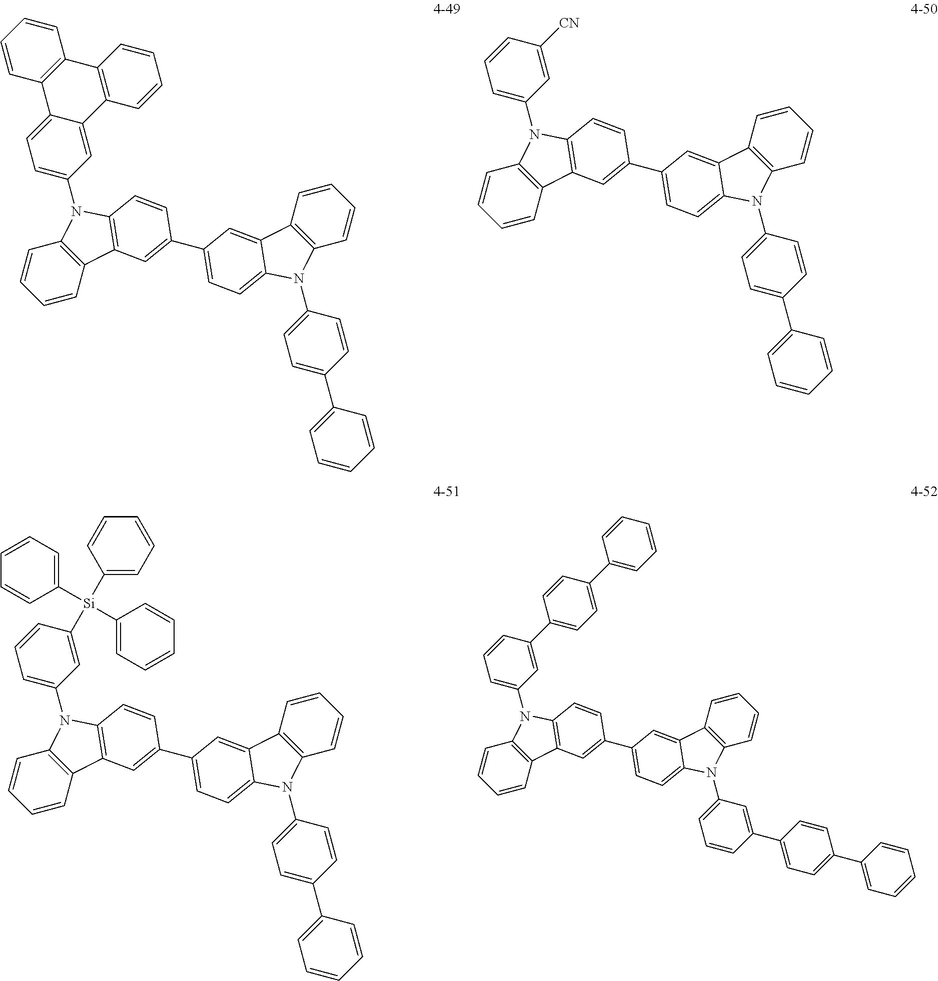 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

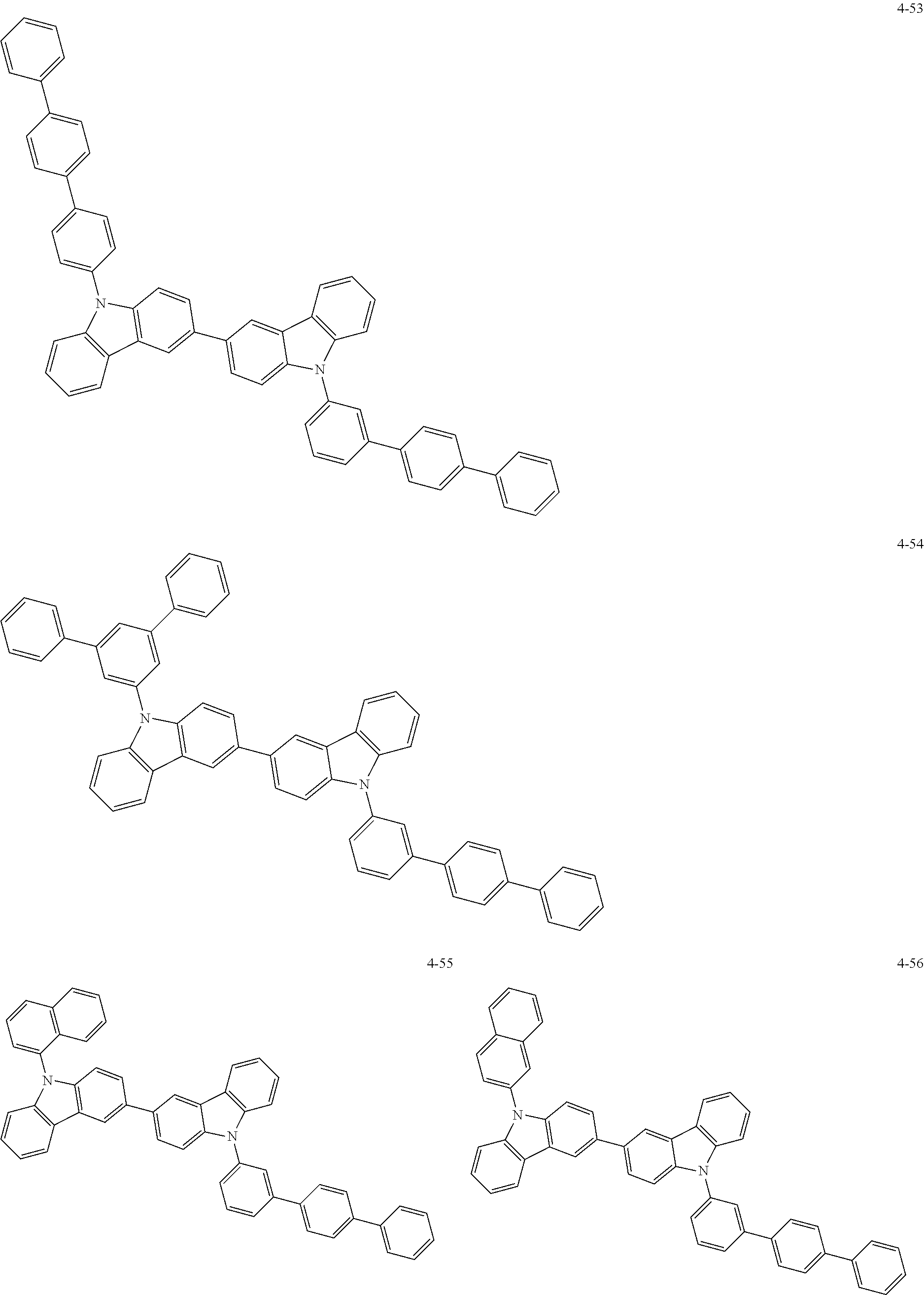 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

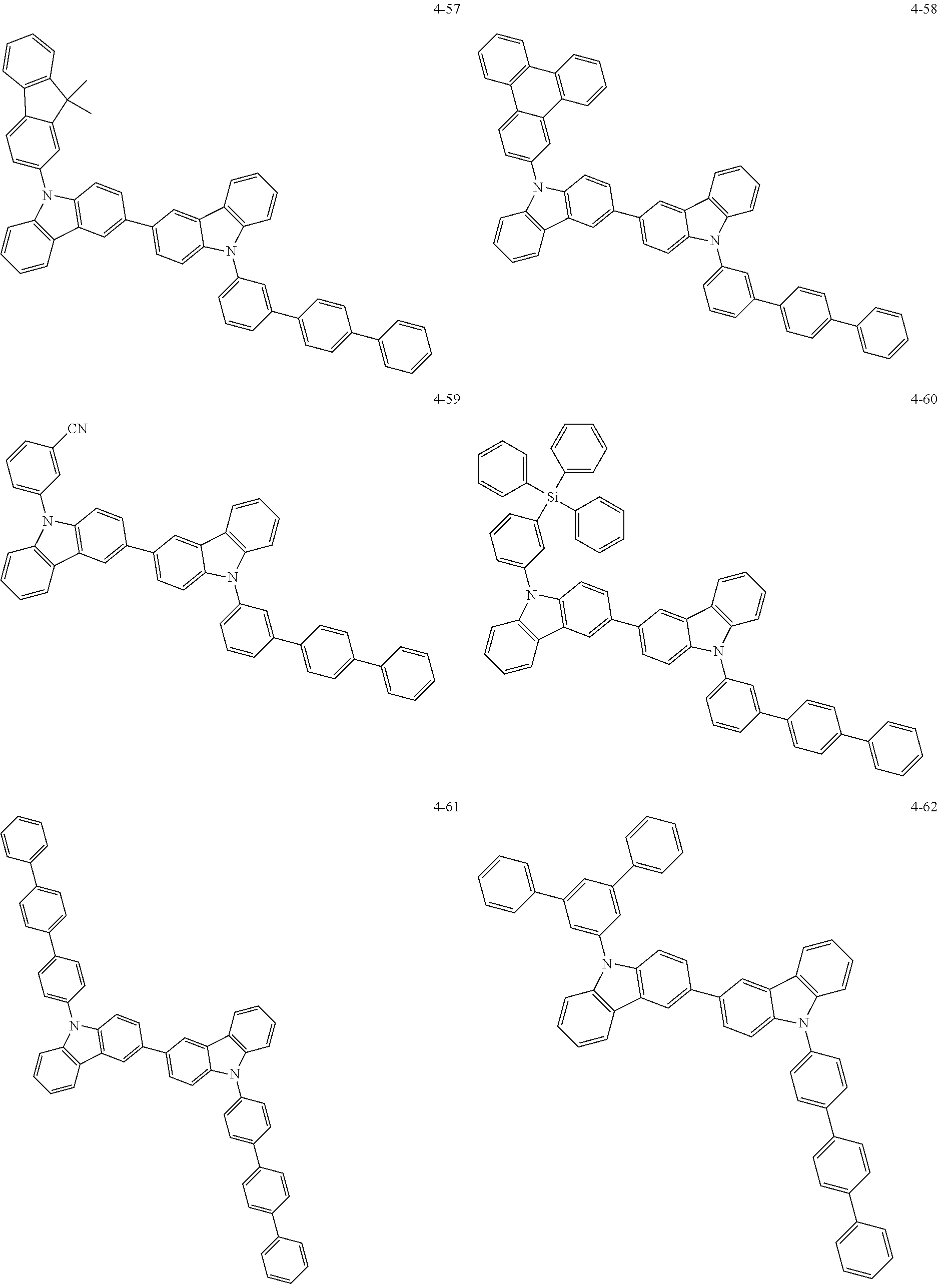

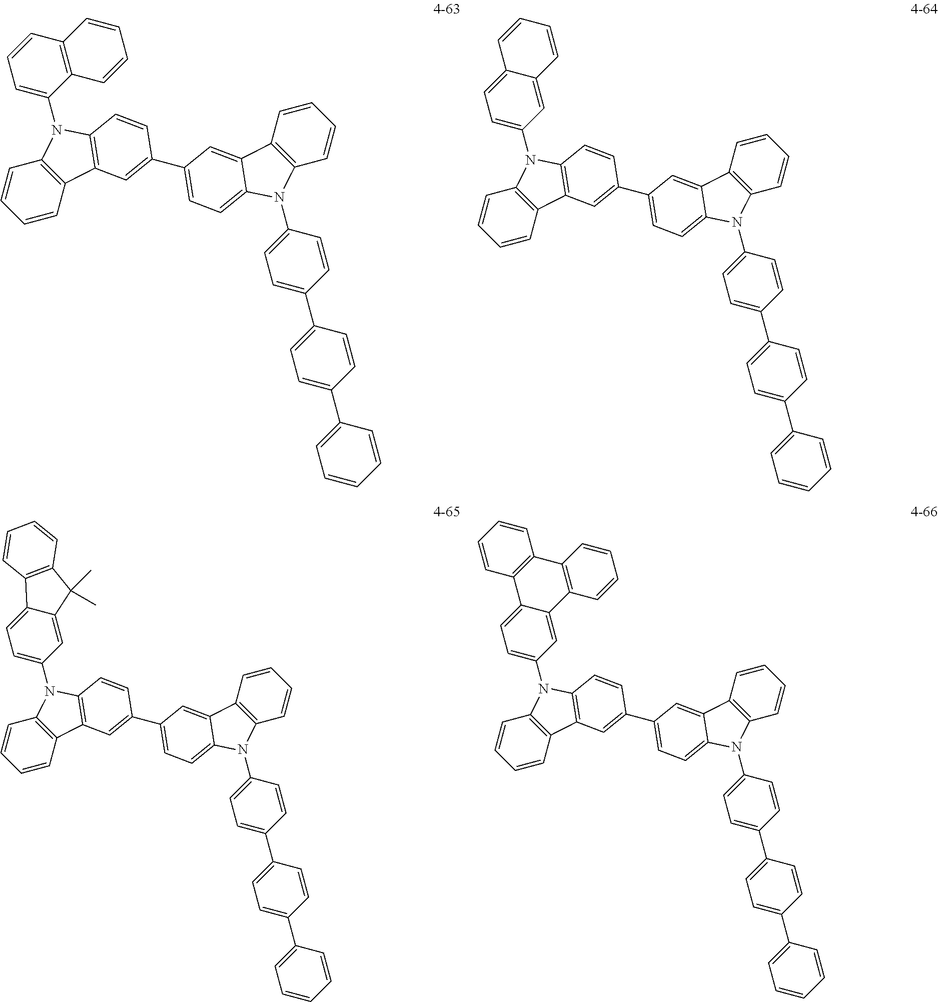 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

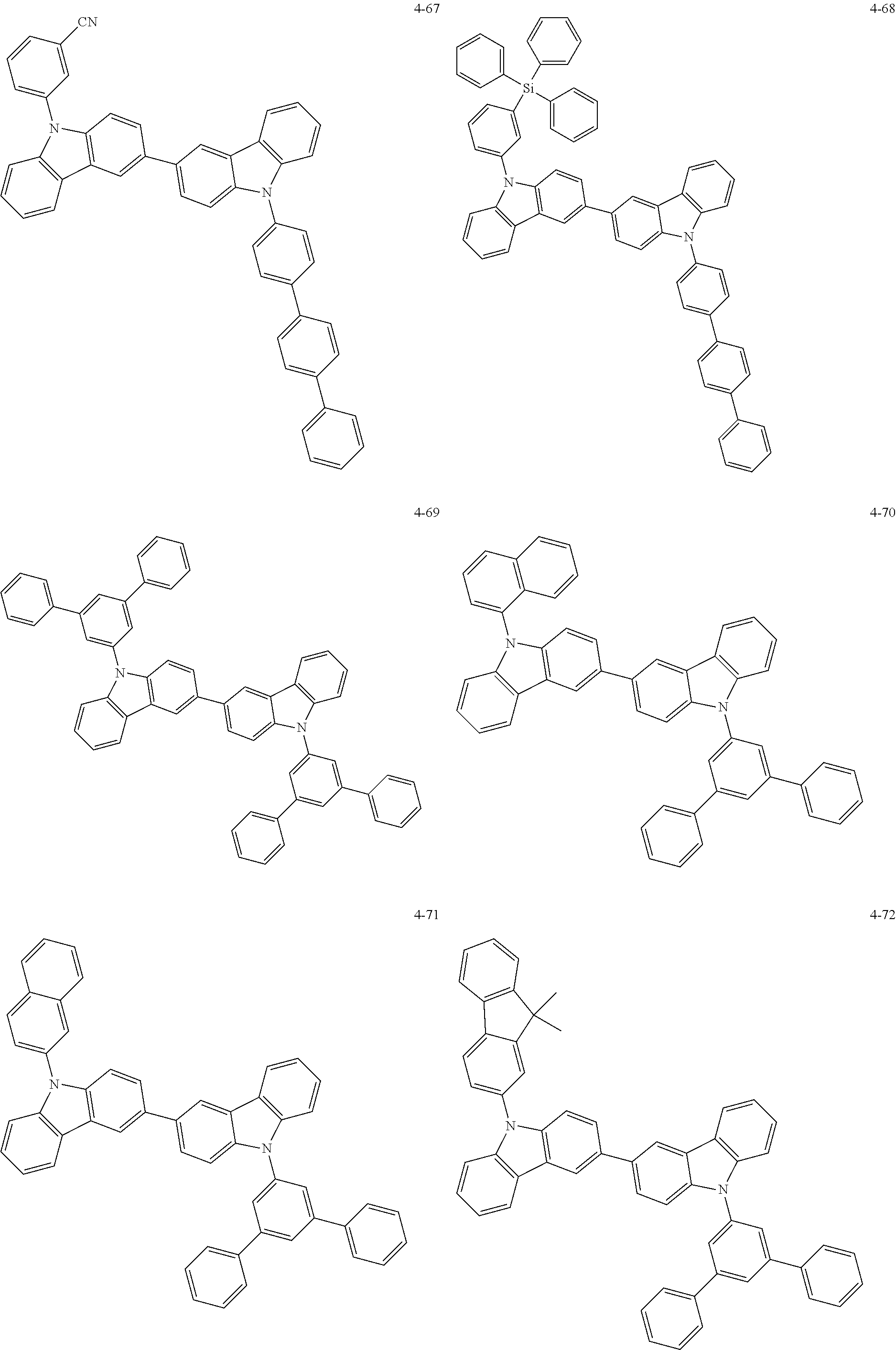

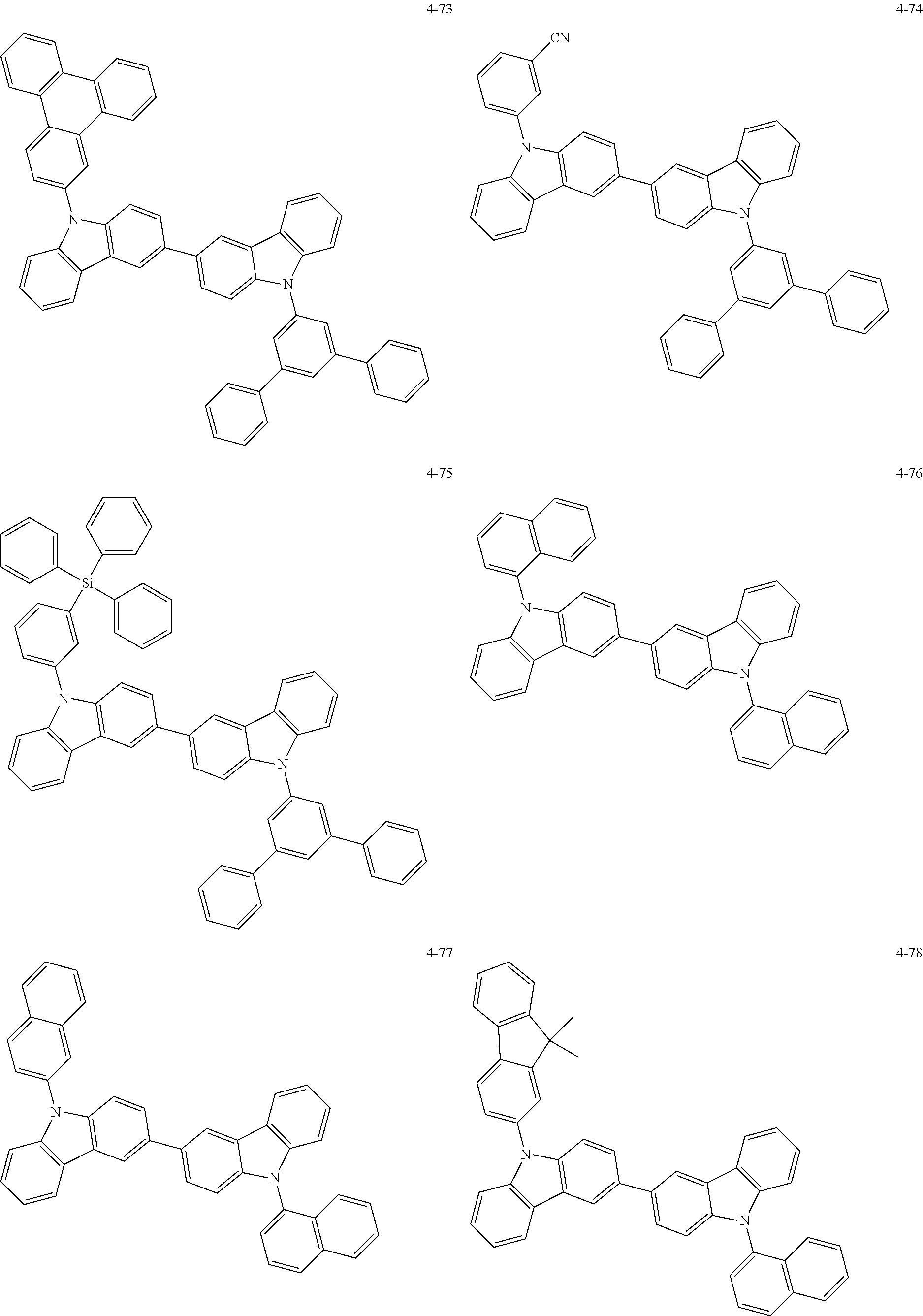

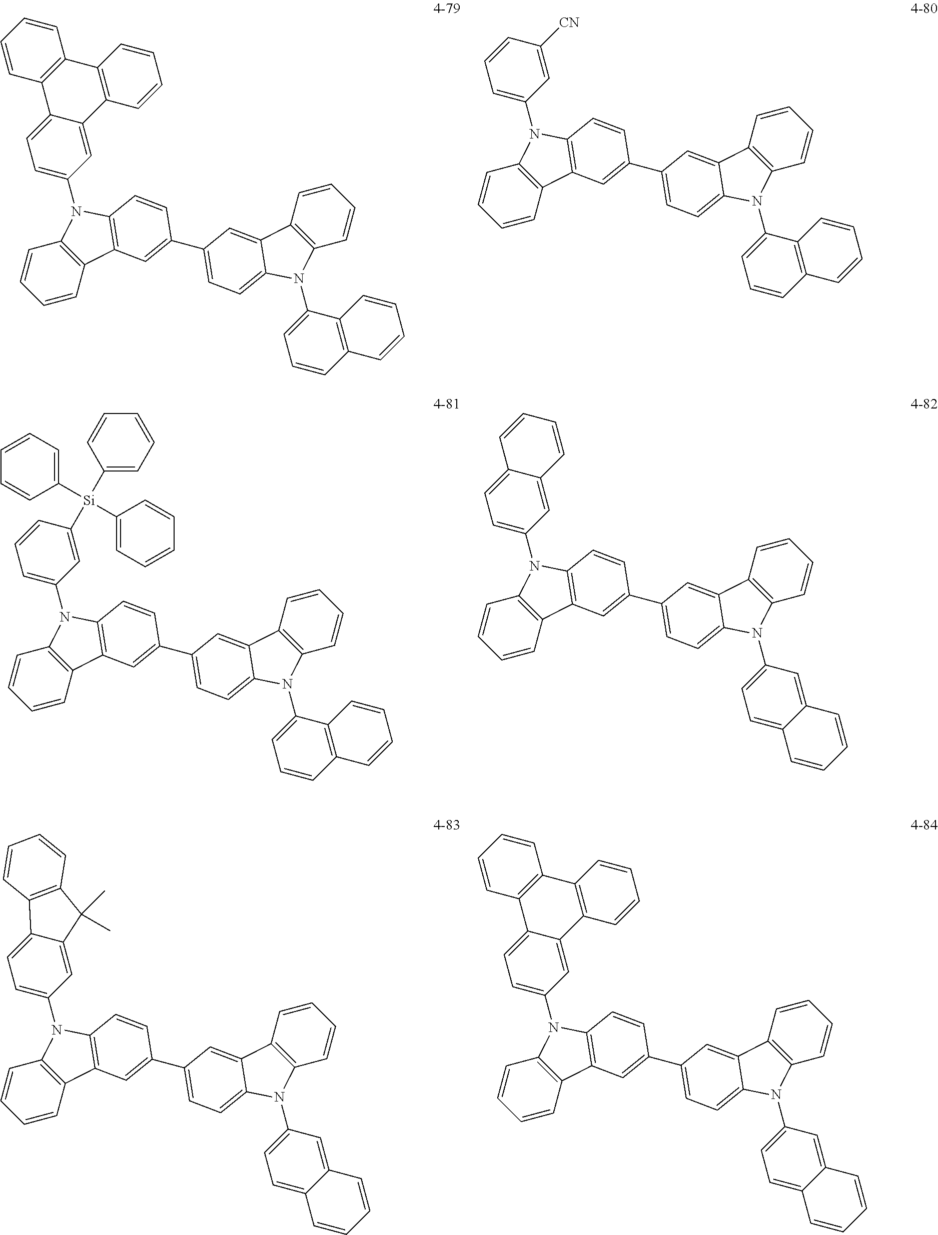

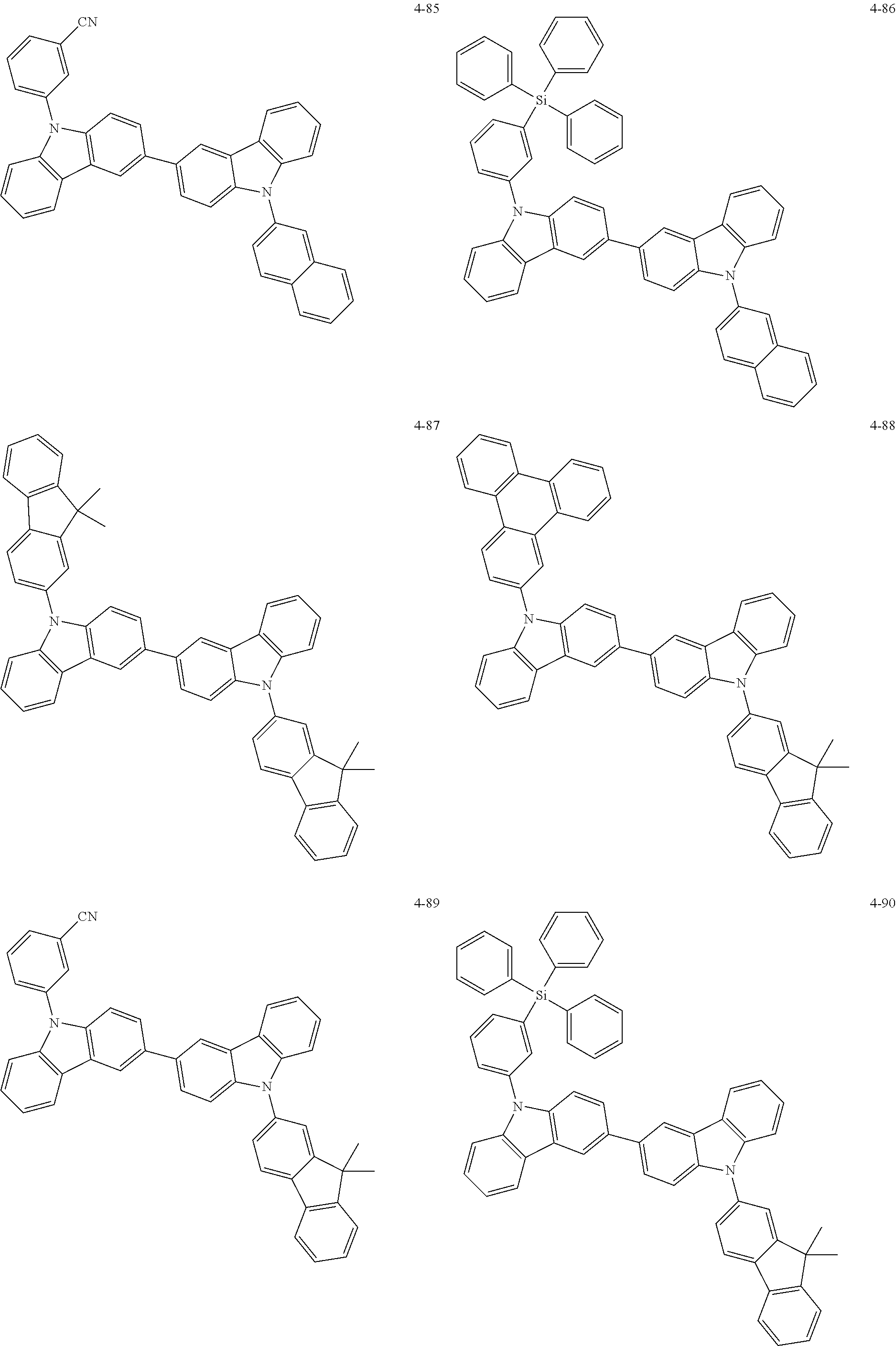

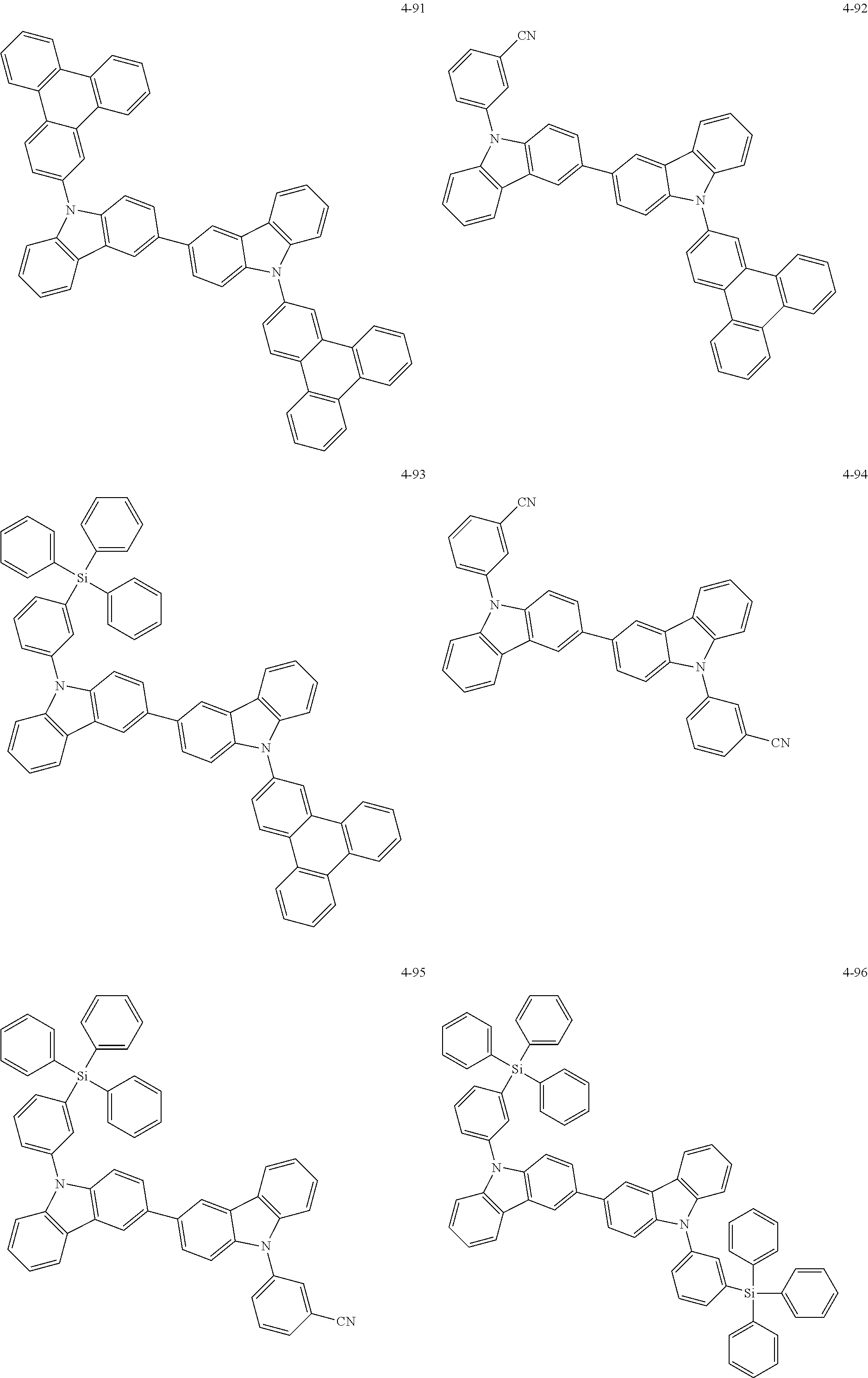

In addition, another embodiment of the present application provides a composition for an organic material layer of an organic light emitting device, the composition comprising the heterocyclic compound represented by Chemical Formula 1 and the heterocyclic compound represented by Chemical Formula 2.

Specific details on the heterocyclic compound represented by Chemical Formula 1 and the heterocyclic compound represented by Chemical Formula 2 are the same as the descriptions provided above.

In the composition, the heterocyclic compound represented by Chemical Formula 1:the heterocyclic compound represented by Chemical Formula 2 may have a weight ratio of 1:10 to 10:1, 1:8 to 8:1, 1:5 to 5:1, or 1:2 to 2:1, however, the weight ratio is not limited thereto.

The composition may be used when forming an organic material of an organic light emitting device, and particularly, may be more preferably used when forming a host of a light emitting layer.

In one embodiment of the present application, the organic material layer comprises the heterocyclic compound represented by Chemical Formula 1 and the heterocyclic compound represented by Chemical Formula 2, and a phosphorescent dopant may be used therewith.

In one embodiment of the present application, the organic material layer comprises the heterocyclic compound represented by Chemical Formula 1 and the heterocyclic compound represented by Chemical Formula 2, and an iridium-based dopant may be used therewith.

As a material of the phosphorescent dopant, those known in the art may be used.

For example, phosphorescent dopant materials represented by LL′MX′, LL′L″M, LMX′X″, L2MX′ and L3M may be used, however, the scope of the present disclosure is not limited to these examples.

Herein, L, L′, L″, X′ and X″ are bidentate ligands different from each other, and M is a metal forming an octahedral complex.

M may comprise iridium, platinum, osmium and the like.

L is an anionic bidentate ligand coordinated to M as the iridium-based dopant by sp2 carbon and heteroatom, and X may function to trap electrons or holes. Nonlimiting examples of L may comprise 2-(1-naphthyl)benzoxazole, (2-phenylbenzoxazole), (2-phenylbenzothiazole), (7,8-benzoquinoline), (thiophene group pyrizine), phenylpyridine, benzothiophene group pyrizine, 3-methoxy-2-phenylpyridine, tolylpyridine and the like. Nonlimiting examples of X′ and X″ may comprise acetylacetonate (acac), hexafluoroacetylacetonate, salicylidene, picolinate, 8-hydroxyquinolinate and the like.

More specific examples of the phosphorescent dopant are described below, however, the phosphorescent dopant is not limited to these examples.

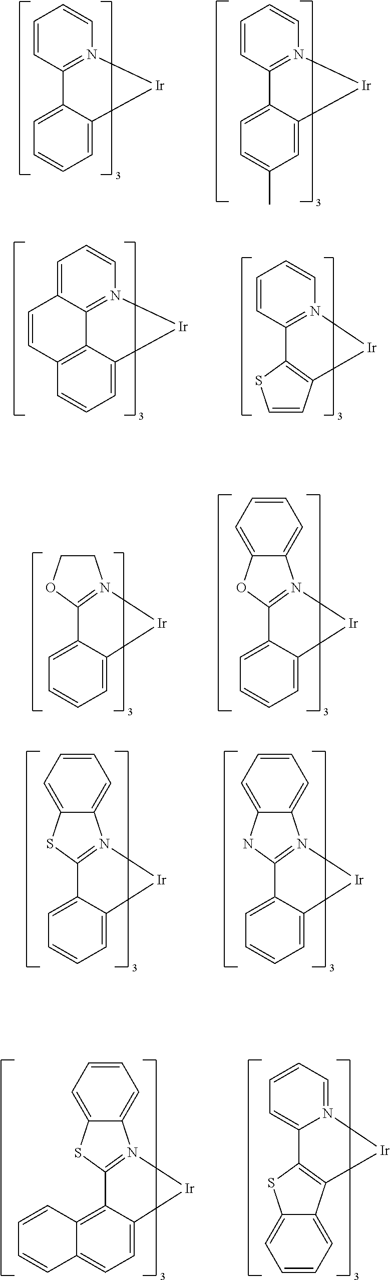

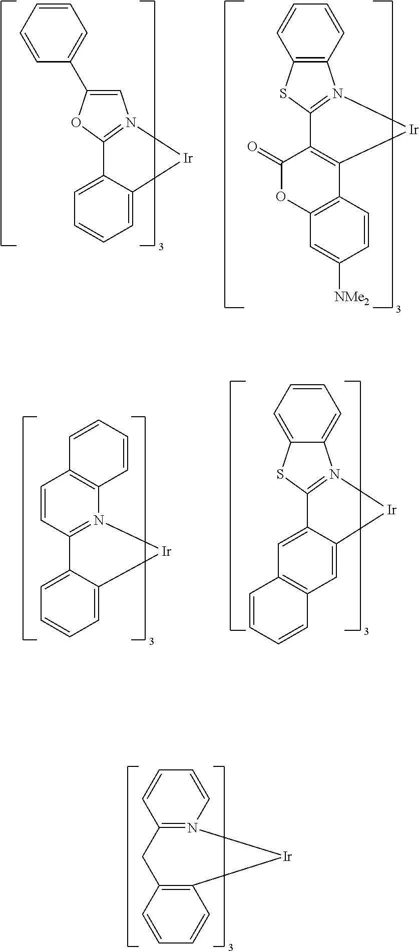

In one embodiment of the present application, as the iridium-based dopant, Ir(ppy)₃ may be used as a green phosphorescent dopant.

In one embodiment of the present application, the content of the dopant may be from 1% to 15%, preferably from 3% to 10% and more preferably from 5% to 10% based on the whole light emitting layer.

In the organic light emitting device of the present disclosure, the organic material layer comprises an electron injection layer or an electron transfer layer, and the electron injection layer or the electron transfer layer may comprise the heterocyclic compound.

In another organic light emitting device, the organic material layer comprises an electron blocking layer or a hole blocking layer, and the electron blocking layer or the hole blocking layer may comprise the heterocyclic compound.

In another organic light emitting device, the organic material layer comprises an electron transfer layer, a light emitting layer or a hole blocking layer, and the electron transfer layer, the light emitting layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

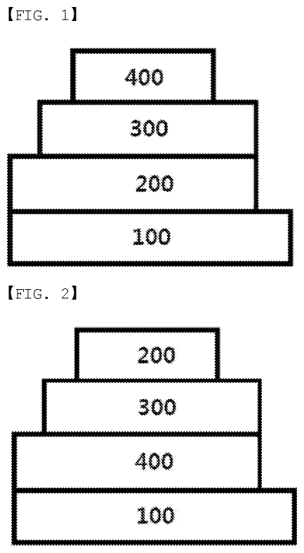

FIGS. 1 to 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

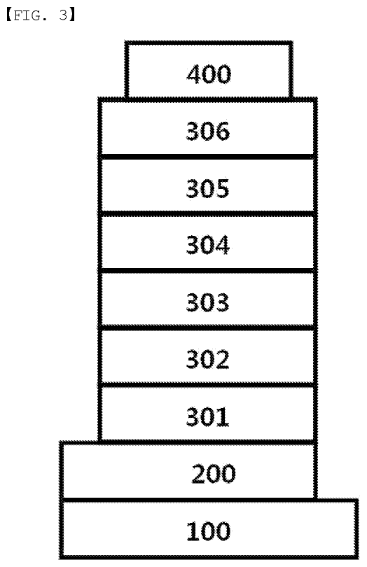

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, other layers except the light emitting layer may not be included, and other necessary functional layers may be further included.

One embodiment of the present application provides a method for manufacturing an organic light emitting device, the method comprising preparing a substrate; forming a first electrode on the substrate; forming one or more organic material layers on the first electrode; and forming a second electrode on the organic material layer, wherein the forming of organic material layers comprises forming one or more organic material layers using the composition for an organic material layer according to one embodiment of the present application.

In the method for manufacturing an organic light emitting device according to one embodiment of the present application, the forming of organic material layers is forming using a method of thermal vacuum deposition after pre-mixing the heterocyclic compound of Chemical Formula 1 and the heterocyclic compound of Chemical Formula 2.

The premixing means mixing materials of the heterocyclic compound of Chemical Formula 1 and the heterocyclic compound of Chemical Formula 2 in advance in one source of supply before depositing on an organic material layer.

The pre-mixed material may be referred to as the composition for an organic material layer according to one embodiment of the present application.

The organic material layer comprising Chemical Formula 1 may further comprise other materials as necessary.

The organic material layer comprising both Chemical Formula 1 and Chemical Formula 2 may further comprise other materials as necessary.

In the organic light emitting device according to one embodiment of the present application, materials other than the compound of Chemical Formula 1 or Chemical Formula 2 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected, and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### Preparation Example

### <Preparation Example 1> Preparation of Compound 1-1

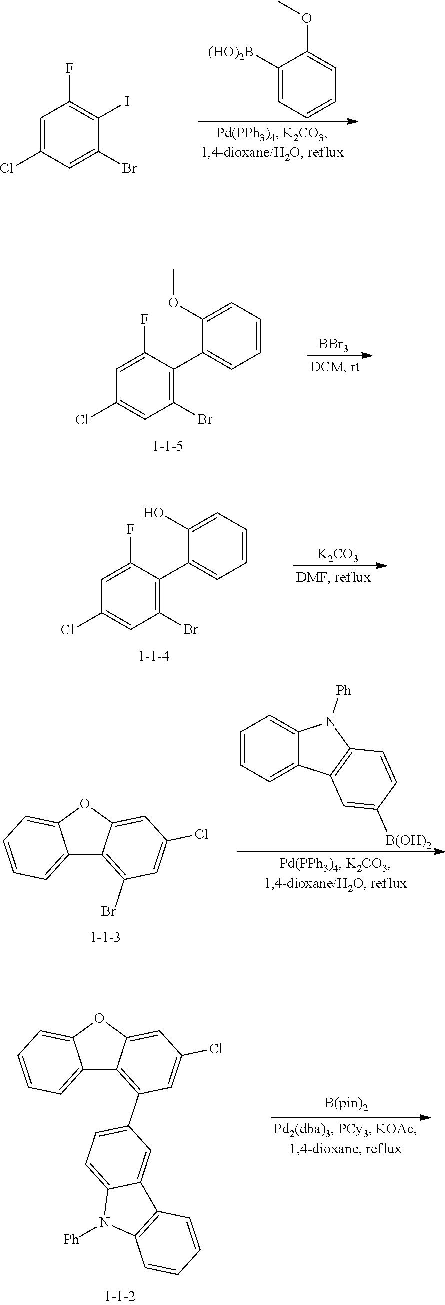

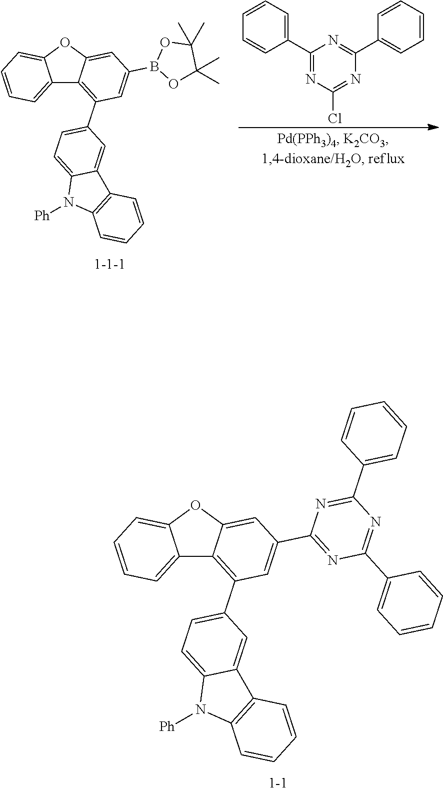

1) Preparation of Compound 1-1-5

After dissolving 1-bromo-5-chloro-3-fluoro-2-iodobenzene (200.0 g, 596.4 mM), (2-methoxyphenyl)boronic acid (82.4 g, 542.2 mM), Pd(PPh)₄ (31.3 g, 27.1 mM) and K₂CO₃ (150.0 g, 1084.4 mM) in 1,4-dioxane/H₂O (1 L/200 mL), the result was refluxed for 24 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified using column chromatography (DCM:Hex=1:10) to obtain target Compound 1-1-5 (137 g, 80%).

2) Preparation of Compound 1-1-4

After dissolving Compound 1-1-5 (82 g, 259.8 mM) and BBr₃ (49 mL, 519.7 mM) in DCM (800 mL), the result was refluxed for 1 hour. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:1) to obtain target Compound 1-1-4 (65.3 g, 83%).

3) Preparation of Compound 1-1-3

After dissolving Compound 1-1-4 (65.3 g, 216.5 mM) and K₂CO₃ (59.9 g, 433.1 mM) in dimethylformamide (DMF) (300 mL), the result was refluxed for 4 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:5), and recrystallized with methanol to obtain target Compound 1-1-3 (54.8 g, 90%).

4) Preparation of Compound 1-1-2

After dissolving Compound 1-1-3 (54.0 g, 191.8 mM), (9-phenyl-9H-carbazol-3-yl)boronic acid (60.6 g, 211.0 mM), Pd(PPh)₄ (11.1 g, 9.6 mM) and K₂CO₃ (53.0 g, 383.6 mM) in 1,4-dioxane/H₂O (300 mL/60 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:10), and recrystallized with methanol to obtain target Compound 1-1-2 (70.6 g, 83%).

5) Preparation of Compound 1-1-1

After dissolving Compound 1-1-2 (70.0 g, 157.7 mM), bis(pinacolato)diboron (60.1 g, 236.6 mM), Pd₂(dba)₃ (14.4 g, 15.8 mM), PCy₃ (8.8 g, 31.5 mM) and KOAc (46.4 g, 473.1 mM) in 1,4-dioxane (700 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3) to obtain target Compound 1-1-1 (50.6 g, 60%).

6) Preparation of Compound 1-1

After dissolving Compound 1-1-1 (50.0 g, 93.4 mM), 2-chloro-4,6-diphenyl-1,3,5-triazine (27.5 g, 102.7 mM), Pd(PPh)₄ (5.4 g, 4.7 mM) and K₂CO₃ (25.8 g, 186.8 mM) in 1,4-dioxane/H₂O (300 mL/60 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:5), and recrystallized with methanol to obtain target Compound 1-1 (36.5 g, 60%).

Target Compound A was synthesized in the same manner as in the preparation of Preparation Example 1 except that Intermediate A of the following Table 1 was used instead of (9-phenyl-9H-carbazol-3-yl)boronic acid, and Intermediate B of the following Table 1 was used instead of 2-chloro-4,6-diphenyl-1,3,5-triazine.

TABLE 1

| Com- | | | | |
|---|---|---|---|---|
| pound | | | | |
| No. | Intermediate A | Intermediate B | Target Compound A | Yield |
| 1-2 | 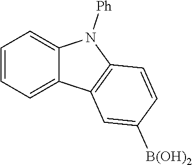 [SMILES: OB(O)c1ccc2c(c1)c1ccccc1n2-c1ccccc1] | 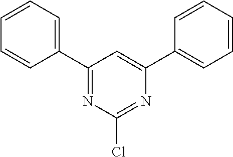 [SMILES: Clc1nc(-c2ccccc2)cc(-c2ccccc2)n1] | 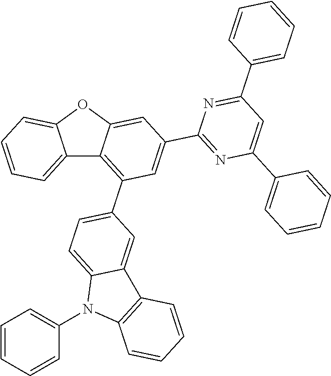 [SMILES: c1ccc(-c2cc(-c3ccccc3)nc(-c3cc(-c4ccc5c(c4)c4ccccc4n5-c4ccccc4)c4c(c3)oc3ccccc34)n2)cc1] | 34% |
| 1-12 | | 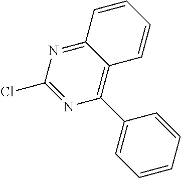 [SMILES: Clc1nc(-c2ccccc2)c2ccccc2n1] | 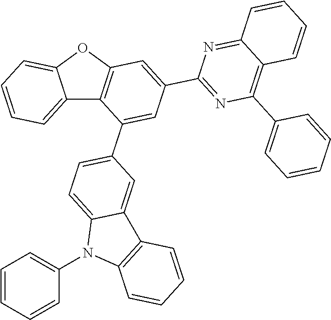 [SMILES: c1ccc(-c2nc(-c3cc(-c4ccc5c(c4)c4ccccc4n5-c4ccccc4)c4c(c3)oc3ccccc34)nc3ccccc23)cc1] | 32% |
| 1-14 | | 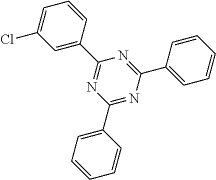 [SMILES: Clc1cccc(-c2nc(-c3ccccc3)nc(-c3ccccc3)n2)c1] | 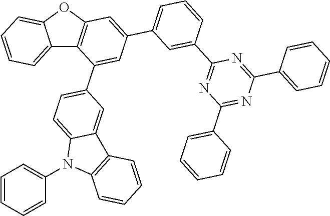 [SMILES: c1ccc(-c2nc(-c3ccccc3)nc(-c3cccc(-c4cc(-c5ccc6c(c5)c5ccccc5n6-c5ccccc5)c5c(c4)oc4ccccc45)c3)n2)cc1] | 36% |
| 1-23 | | 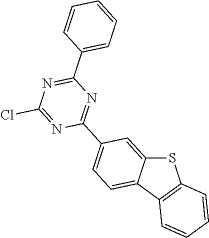 [SMILES: Clc1nc(-c2ccccc2)nc(-c2ccc3c(c2)sc2ccccc23)n1] | 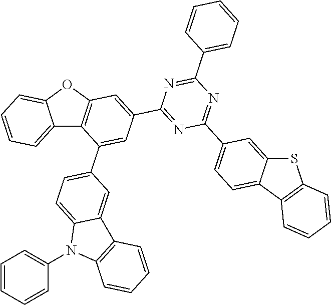 [SMILES: c1ccc(-c2nc(-c3cc(-c4ccc5c(c4)c4ccccc4n5-c4ccccc4)c4c(c3)oc3ccccc34)nc(-c3ccc4c(c3)sc3ccccc34)n2)cc1] | 33% |
| 1-29 | | 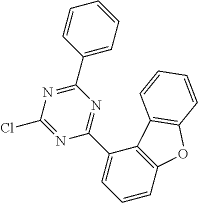 [SMILES: Clc1nc(-c2ccccc2)nc(-c2cccc3oc4ccccc4c23)n1] | 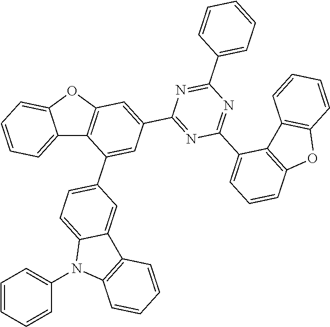 [SMILES: c1ccc(-c2nc(-c3cc(-c4ccc5c(c4)c4ccccc4n5-c4ccccc4)c4c(c3)oc3ccccc34)nc(-c3cccc4oc5ccccc5c34)n2)cc1] | 35% |
| 1-34 | | 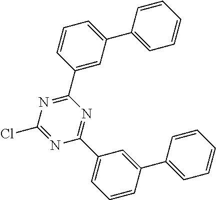 [SMILES: Clc1nc(-c2cccc(-c3ccccc3)c2)nc(-c2cccc(-c3ccccc3)c2)n1] | 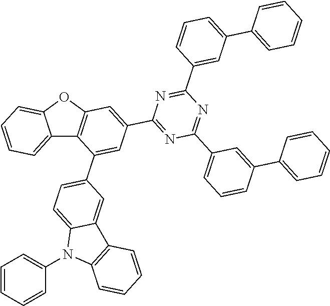 [SMILES: c1ccc(-c2cccc(-c3nc(-c4cccc(-c5ccccc5)c4)nc(-c4cc(-c5ccc6c(c5)c5ccccc5n6-c5ccccc5)c5c(c4)oc4ccccc45)n3)c2)cc1] | 34% |
| 1-61 | 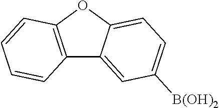 [SMILES: OB(O)c1ccc2oc3ccccc3c2c1] | 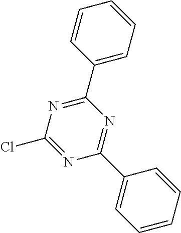 [SMILES: Clc1nc(-c2ccccc2)nc(-c2ccccc2)n1] | 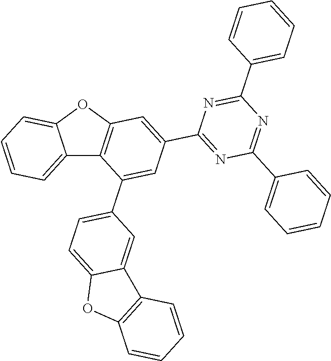 [SMILES: c1ccc(-c2nc(-c3ccccc3)nc(-c3cc(-c4ccc5oc6ccccc6c5c4)c4c(c3)oc3ccccc34)n2)cc1] | 32% |
| 1-76 | 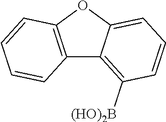 [SMILES: OB(O)c1cccc2oc3ccccc3c12] | | 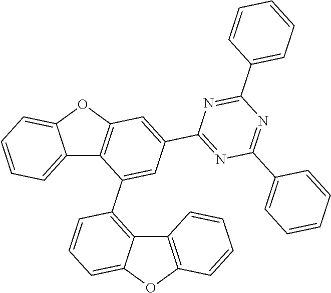 [SMILES: c1ccc(-c2nc(-c3ccccc3)nc(-c3cc(-c4cccc5oc6ccccc6c45)c4c(c3)oc3ccccc34)n2)cc1] | 36% |
| 1-81 | 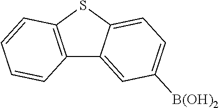 [SMILES: OB(O)c1ccc2sc3ccccc3c2c1] | | 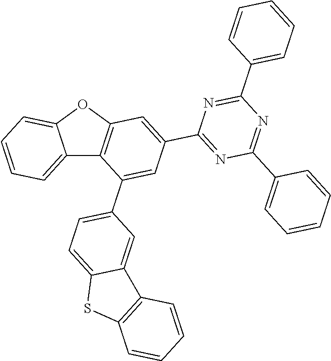 [SMILES: c1ccc(-c2nc(-c3ccccc3)nc(-c3cc(-c4ccc5sc6ccccc6c5c4)c4c(c3)oc3ccccc34)n2)cc1] | 33% |
| 1-101 | 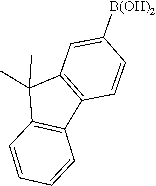 [SMILES: CC1(C)c2ccccc2-c2ccc(B(O)O)cc21] | | 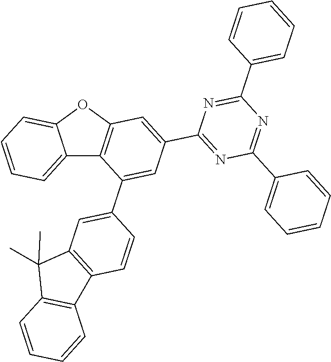 [SMILES: CC1(C)c2ccccc2-c2ccc(-c3cc(-c4nc(-c5ccccc5)nc(-c5ccccc5)n4)cc4oc5ccccc5c34)cc21] | 37% |
| 1-115 | 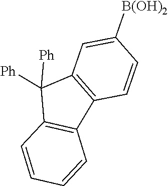 [SMILES: OB(O)c1ccc2c(c1)C(c1ccccc1)(c1ccccc1)c1ccccc1-2] | | 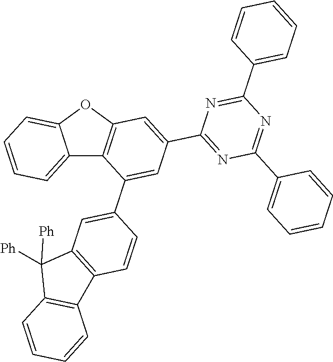 [SMILES: c1ccc(-c2nc(-c3ccccc3)nc(-c3cc(-c4ccc5c(c4)C(c4ccccc4)(c4ccccc4)c4ccccc4-5)c4c(c3)oc3ccccc34)n2)cc1] | 36% |
| 1-117 | 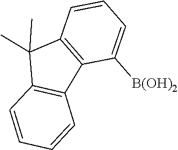 [SMILES: CC1(C)c2ccccc2-c2c(B(O)O)cccc21] | | 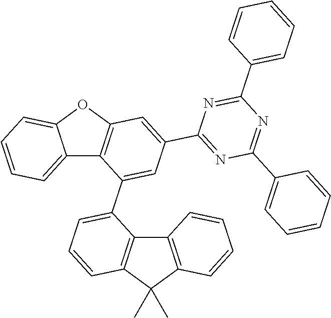 [SMILES: CC1(C)c2ccccc2-c2c(-c3cc(-c4nc(-c5ccccc5)nc(-c5ccccc5)n4)cc4oc5ccccc5c34)cccc21] | 34% |

### <Preparation Example 2> Preparation of Compound 1-121

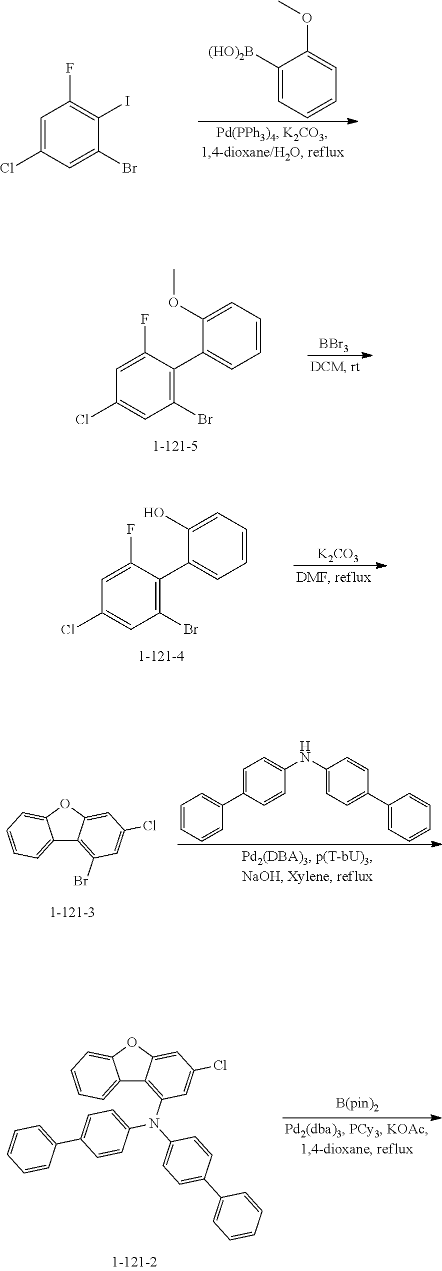

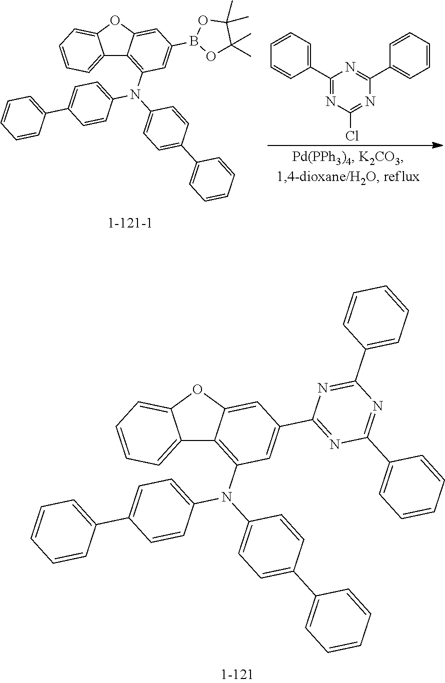

1) Preparation of Compound 1-121-2

After dissolving Compound 1-121-3 (11.0 g, 39.1 mM), di([1,1′-biphenyl]-4-yl)amine (11.4 g, 35.5 mM), Pd₂(dba)₃ (1.6 g, 1.8 mM), P(t-Bu)₃ (1.6 mL, 3.6 mM) and NaOH (2.8 g, 71.0 mM) in 1,4-dioxane (200 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-121-2 (15.8 g, 85%).

2) Preparation of Compound 1-121-1

After dissolving Compound 1-121-2 (15.0 g, 28.7 mM), bis(pinacolato)diboron (10.9 g, 43.1 mM), Pd₂ (dba)₃ (2.6 g, 2.9 mM), PCy₃ (1.6 g, 5.7 mM) and KOAc (8.4 g, 86.1 mM) in 1,4-dioxane (300 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-121-1 (15.7 g, 89%).

3) Preparation of Compound 1-121

After dissolving Compound 1-121-1 (15.0 g, 24.4 mM), 2-chloro-4,6-diphenyl-1,3,5-triazine (7.2 g, 26.8 mM), Pd(PPh)₄ (1.4 g, 1.2 mM) and K₂CO₃ (6.7 g, 48.8 mM) in 1,4-dioxane/H₂O (200 mL/40 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-121 (14.3 g, 82%).

Target Compound A was synthesized in the same manner as in the preparation of Preparation Example 2 except that Intermediate A of the following Table 2 was used instead of di([1,1′-biphenyl]-4-yl)amine, and Intermediate B of the following Table 2 was used instead of 2-chloro-4,6-diphenyl-1,3,5-triazine.

TABLE 2

| Com- | | | | |
|---|---|---|---|---|
| pound | | | | |
| No. | Intermediate A | Intermediate B | Target Compound A | Yield |
| 1-135 | 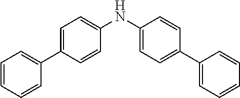 [SMILES: c1ccc(-c2ccc(Nc3ccc(-c4ccccc4)cc3)cc2)cc1] | 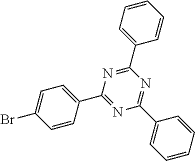 [SMILES: Brc1ccc(-c2nc(-c3ccccc3)nc(-c3ccccc3)n2)cc1] | 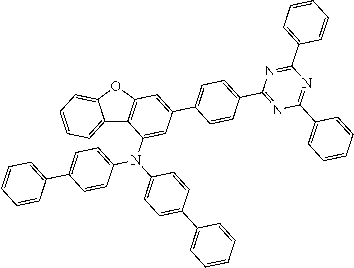 [SMILES: c1ccc(-c2ccc(N(c3ccc(-c4ccccc4)cc3)c3cc(-c4ccc(-c5nc(-c6ccccc6)nc(-c6ccccc6)n5)cc4)cc4oc5ccccc5c34)cc2)cc1] | 36% |

### <Preparation Example 3> Preparation of Compound 1-172

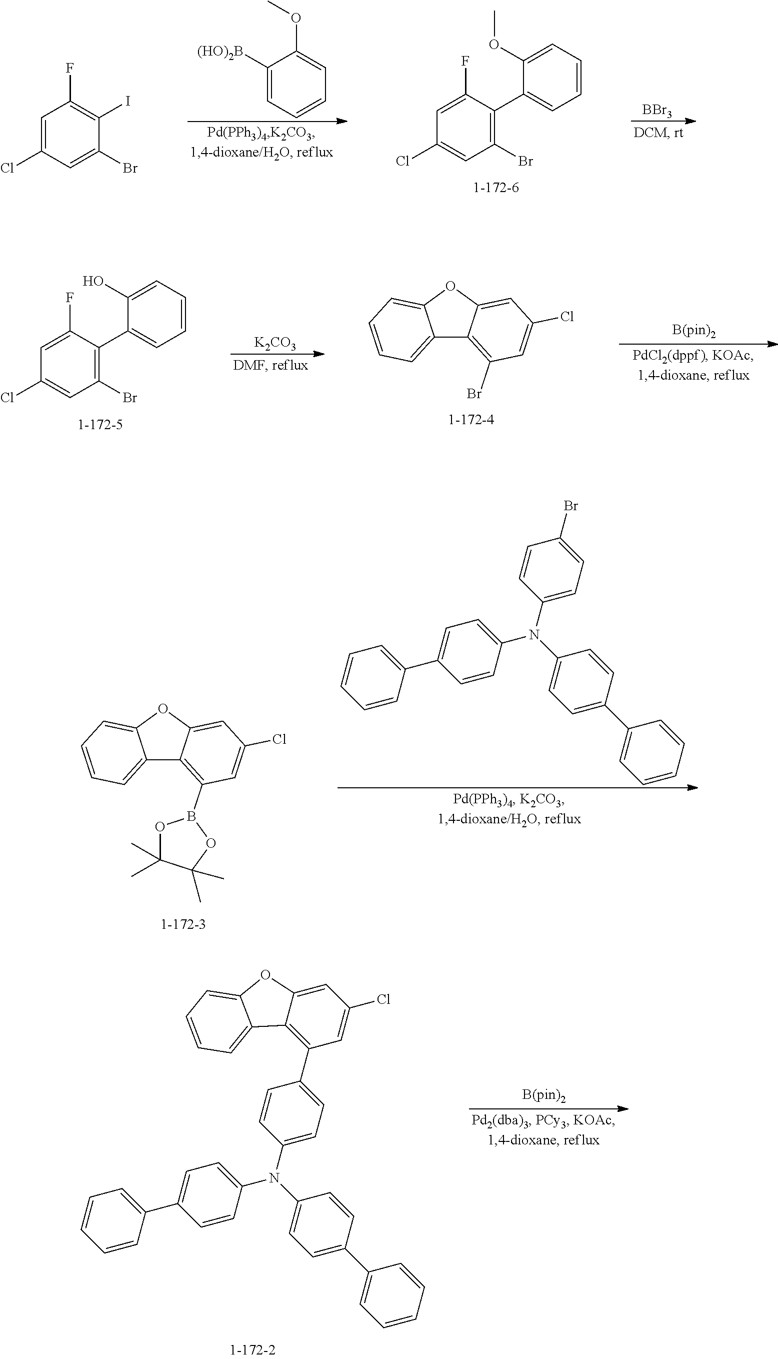

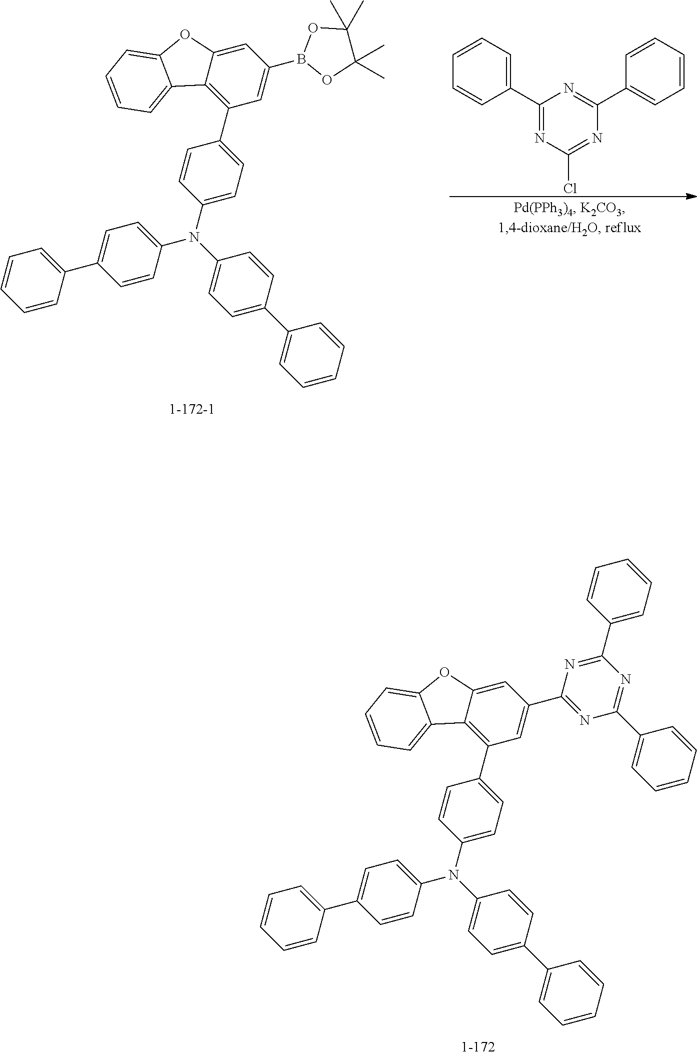 [SMILES: CC1(C)OB(c2cc(-c3ccc(N(c4ccc(-c5ccccc5)cc4)c4ccc(-c5ccccc5)cc4)cc3)c3c(c2)oc2ccccc23)OC1(C)C ; Clc1nc(-c2ccccc2)nc(-c2ccccc2)n1 ; c1ccc(-c2ccc(N(c3ccc(-c4ccccc4)cc3)c3ccc(-c4cc(-c5nc(-c6ccccc6)nc(-c6ccccc6)n5)cc5oc6ccccc6c45)cc3)cc2)cc1]

1) Preparation of Compound 1-172-3

After dissolving Compound 1-172-4 (15.0 g, 53.3 mM), bis(pinacolato)diboron (20.3 g, 80.0 mM), PdCl₂(dppf) (3.9 g, 5.3 mM) and KOAc (15.7 g, 159.9 mM) in 1,4-dioxane (200 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-172-3 (14.9 g, 85%).

2) Preparation of Compound 1-172-2

After dissolving Compound 1-172-3 (14.0 g, 42.6 mM), N-([1,1′-biphenyl]-4-yl)-N-(4-bromophenyl)-[1,1′-biphenyl]-4-amine (20.3 g, 42.6 mM), Pd(PPh)₄ (2.5 g, 2.1 mM) and K₂CO₃ (11.8 g, 85.2 mM) in 1,4-dioxane/H₂O (500 mL/100 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:5), and recrystallized with methanol to obtain target Compound 1-172-2 (20.9 g, 82%).

3) Preparation of Compound 1-172-1

After dissolving Compound 1-172-2 (20.0 g, 33.4 mM), bis(pinacolato)diboron (12.7 g, 50.1 mM), Pd₂(dba)₃ (3.1 g, 3.3 mM), PCy₃ (1.9 g, 6.7 mM) and KOAc (9.8 g, 100.2 mM) in 1,4-dioxane (200 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-172-1 (20.5 g, 89%).

4) Preparation of Compound 1-172

After dissolving Compound 1-172-1 (20.0 g, 29.0 mM), 2-chloro-4,6-diphenyl-1,3,5-triazine (7.8 g, 29.0 mM), Pd(PPh)₄ (1.7 g, 1.5 mM) and K₂CO₃ (8.0 g, 58.0 mM) in 1,4-dioxane/H₂O (300 mL/60 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-172 (18.9 g, 82%).

Target Compound A was synthesized in the same manner as in the preparation of Preparation Example 3 except that Intermediate A of the following Table 3 was used instead of N-([1,1′-biphenyl]-4-yl)-N-(4-bromophenyl)-[1,1′-biphenyl]-4-amine, and Intermediate B of the following Table 3 was used instead of 2-chloro-4,6-diphenyl-1,3,5-triazine.

TABLE 3

| Compound | | | | |
|---|---|---|---|---|
| No. | Intermediate A | Intermediate B | Target Compound A | Yield |
| 1-173 | 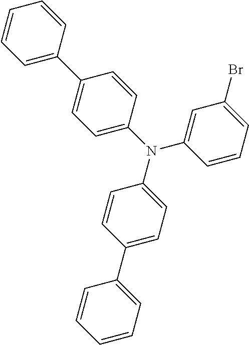 [SMILES: Brc1cccc(N(c2ccc(-c3ccccc3)cc2)c2ccc(-c3ccccc3)cc2)c1] | | 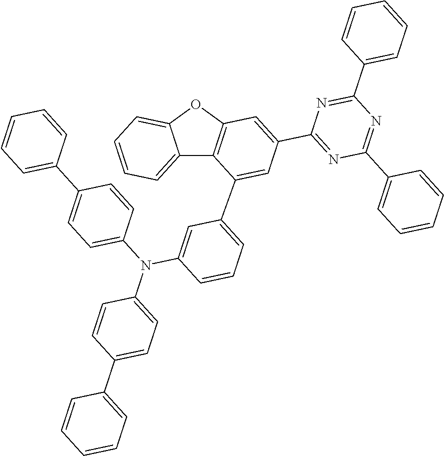 [SMILES: c1ccc(-c2ccc(N(c3ccc(-c4ccccc4)cc3)c3cccc(-c4cc(-c5nc(-c6ccccc6)nc(-c6ccccc6)n5)cc5oc6ccccc6c45)c3)cc2)cc1] | 36% |
| 1-192 | 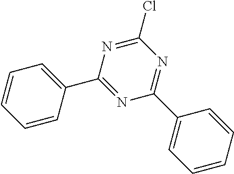 [SMILES: Clc1nc(-c2ccccc2)nc(-c2ccccc2)n1] | 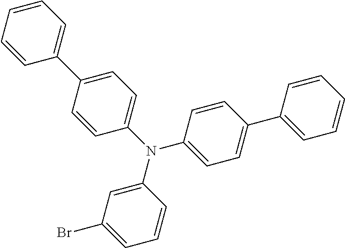 [SMILES: Brc1cccc(N(c2ccc(-c3ccccc3)cc2)c2ccc(-c3ccccc3)cc2)c1] | 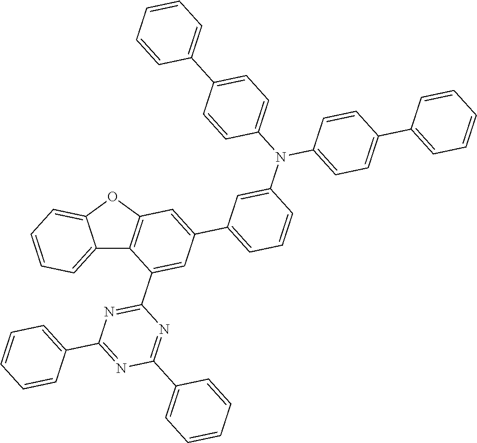 [SMILES: c1ccc(-c2ccc(N(c3ccc(-c4ccccc4)cc3)c3cccc(-c4cc(-c5nc(-c6ccccc6)nc(-c6ccccc6)n5)c5c(c4)oc4ccccc45)c3)cc2)cc1] | 36% |
| 1-202 | 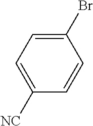 | 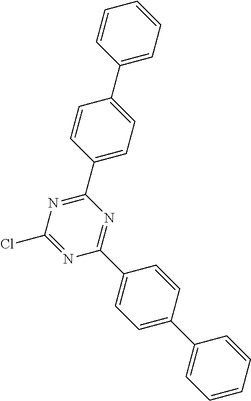 [SMILES: Clc1nc(-c2ccc(-c3ccccc3)cc2)nc(-c2ccc(-c3ccccc3)cc2)n1] | 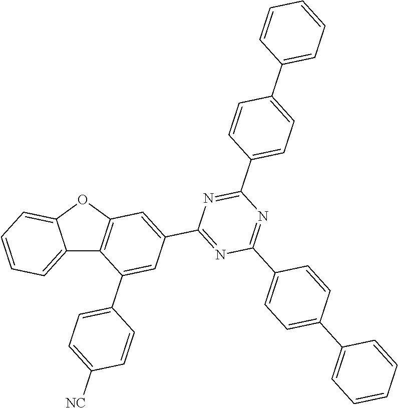 [SMILES: N#Cc1ccc(-c2cc(-c3nc(-c4ccc(-c5ccccc5)cc4)nc(-c4ccc(-c5ccccc5)cc4)n3)cc3oc4ccccc4c23)cc1] | 33% |
| 1-212 | 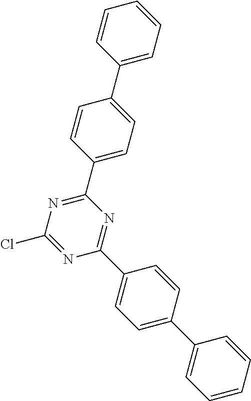 [SMILES: Clc1nc(-c2ccc(-c3ccccc3)cc2)nc(-c2ccc(-c3ccccc3)cc2)n1] | 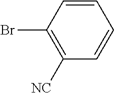 | 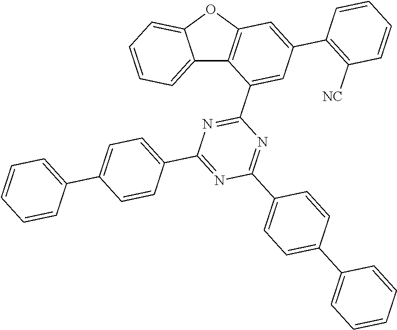 [SMILES: N#Cc1ccccc1-c1cc(-c2nc(-c3ccc(-c4ccccc4)cc3)nc(-c3ccc(-c4ccccc4)cc3)n2)c2c(c1)oc1ccccc12] | 37% |

### <Preparation Example 4> Preparation of Compound 1-178

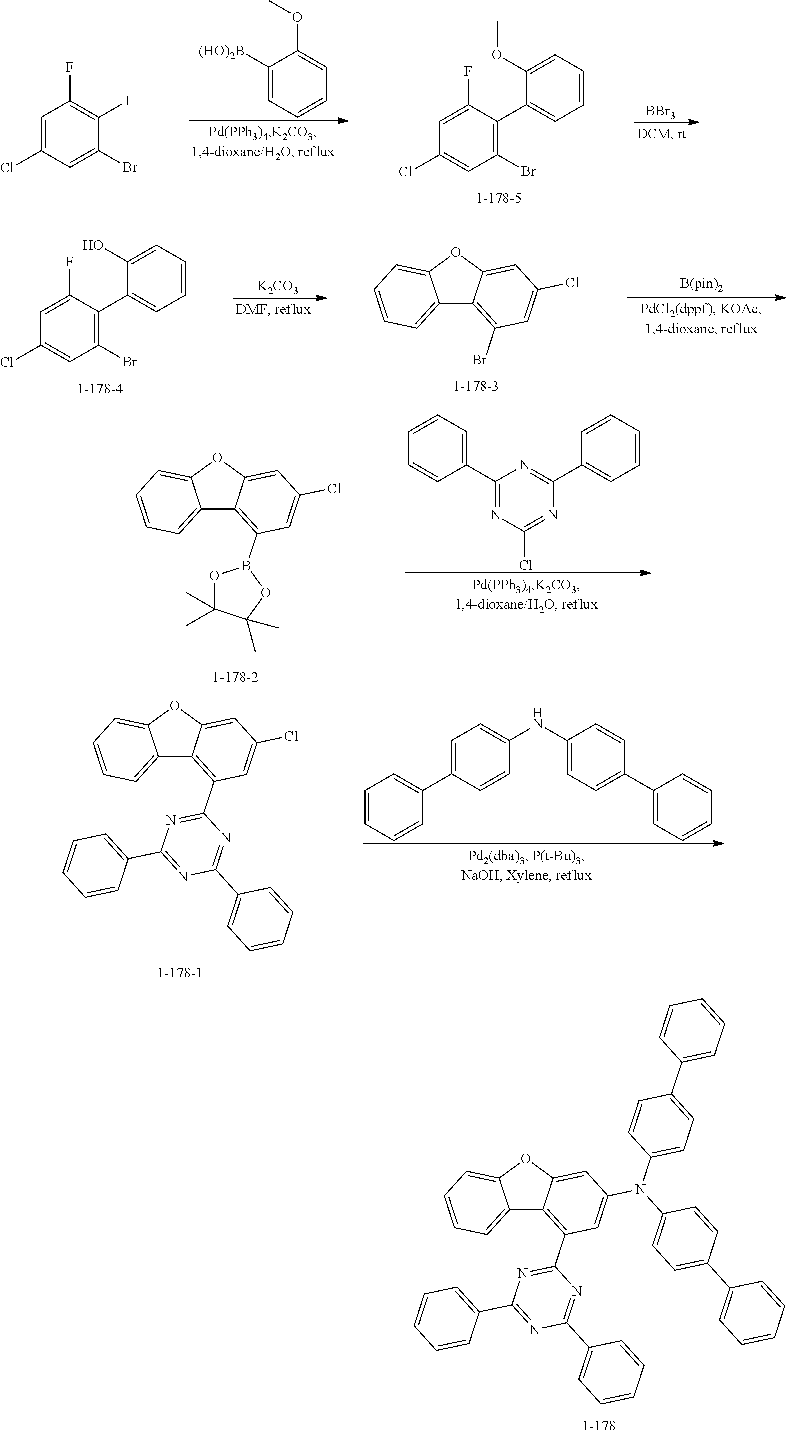

1) Preparation of Compound 1-178

After dissolving Compound 1-178-1 (10.0 g, 23.0 mM), di([1,1′-biphenyl]-4-yl)amine (6.7 g, 20.9 mM), Pd₂(dba)₃ (1.0 g, 1.0 mM), P(t-Bu)₃ (1.0 mL, 2.1 mM) and NaOH (1.7 g, 41.8 mM) in 1,4-dioxane (200 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-178 (12.9 g, 86%).

Target Compound A was synthesized in the same manner as in the preparation of Preparation Example 4 except that Intermediate A of the following Table 4 was used instead of 2-chloro-4,6-diphenyl-1,3,5-triazine, and Intermediate B of the following Table 4 was used instead of di([1,1′-biphenyl]-4-yl)amine.

TABLE 4

| Compound | | | | |
|---|---|---|---|---|
| No. | Intermediate A | Intermediate B | Target Compound A | Yield |
| 1-188 | 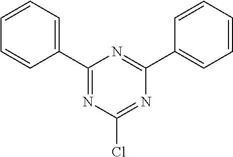 [SMILES: Clc1nc(-c2ccccc2)nc(-c2ccccc2)n1] | 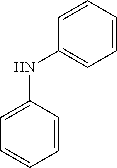 [SMILES: c1ccc(Nc2ccccc2)cc1] | 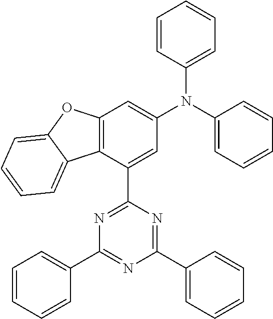 [SMILES: c1ccc(-c2nc(-c3ccccc3)nc(-c3cc(N(c4ccccc4)c4ccccc4)cc4oc5ccccc5c34)n2)cc1] | 36% |

### <Preparation Example 5> Preparation of Compound 1-197

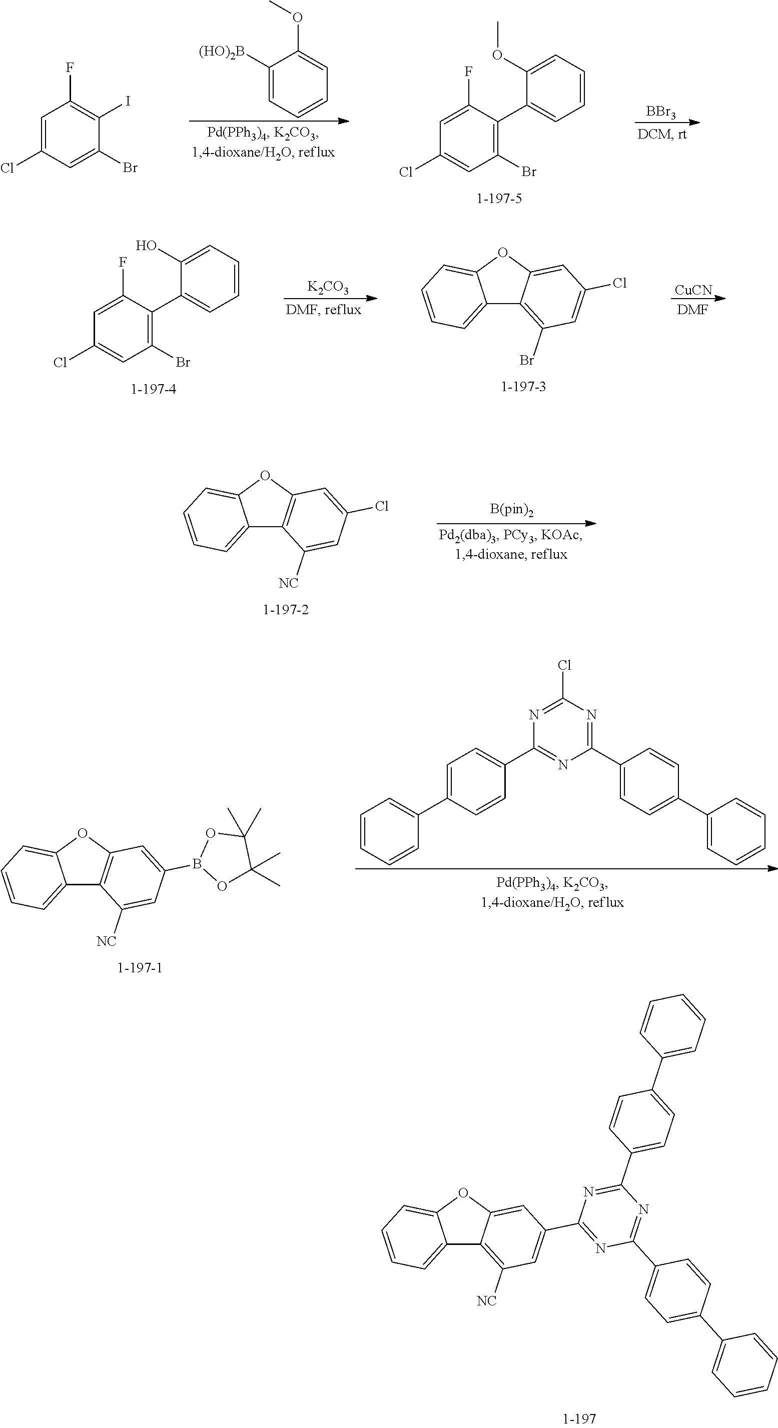

1) Preparation of Compound 1-197-2

After dissolving Compound 1-197-3 (30 g, 131.8 mM) and copper(I) cyanide (23.6 g, 263.6 mM) in DMF (300 mL), the result was refluxed for 24 hours. After the reaction was completed, the copper (I) cyanide was filtered, and the result was extracted by introducing distilled water and DCM thereto at room temperature. After drying the organic layer with MgSO₄, the solvent was removed using a rotary evaporator. The reaction material was purified using column chromatography (DCM:Hex=1:3), and recrystallized with methanol to obtain target Compound 1-197-2 (24.9 g, 83%).

2) Preparation of Compound 1-197-1

After dissolving Compound 1-197-2 (24.0 g, 105.4 mM), bis(pinacolato)diboron (53.5 g, 210.8 mM), Pd₂(dba)₃ (4.9 g, 5.3 mM), PCy₃ (5.9 g, 21.1 mM) and KOAc (31.0 g, 316.2 mM) in 1,4-dioxane (200 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-197-1 (30.0 g, 89%).

3) Preparation of Compound 1-197

After dissolving Compound 1-197-1 (15 g, 47.0 mM), 2-chloro-4,6-diphenyl-1,3,5-triazine (19.7 g, 47.0 mM), Pd(PPh)₄ (2.7 g, 2.4 mM) and K₂CO₃ (13.0 g, 94.0 mM) in 1,4-dioxane/H₂O (300 mL/60 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 1-197 (22.2 g, 82%).

Target Compound A was synthesized in the same manner as in the preparation of Preparation Example 5 except that Intermediate A of the following Table 5 was used instead of copper(I) cyanide, and Intermediate B of the following Table 5 was used instead of 2,4-di([1,1′-biphenyl]-4-yl)-6-chloro-1,3,5-triazine.

TABLE 5

| Compound | | | | |
|---|---|---|---|---|
| No. | Intermediate A | Intermediate B | Target Compound A | Yield |
| 1-200 | CuCN | 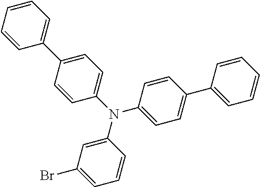 [SMILES: Brc1cccc(N(c2ccc(-c3ccccc3)cc2)c2ccc(-c3ccccc3)cc2)c1] | 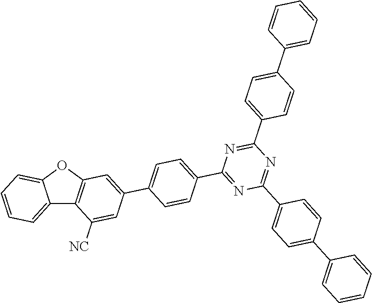 [SMILES: N#Cc1cc(-c2ccc(-c3nc(-c4ccc(-c5ccccc5)cc4)nc(-c4ccc(-c5ccccc5)cc4)n3)cc2)cc2oc3ccccc3c12] | 36% |

### <Preparation Example 6> Preparation of Compound 1-205

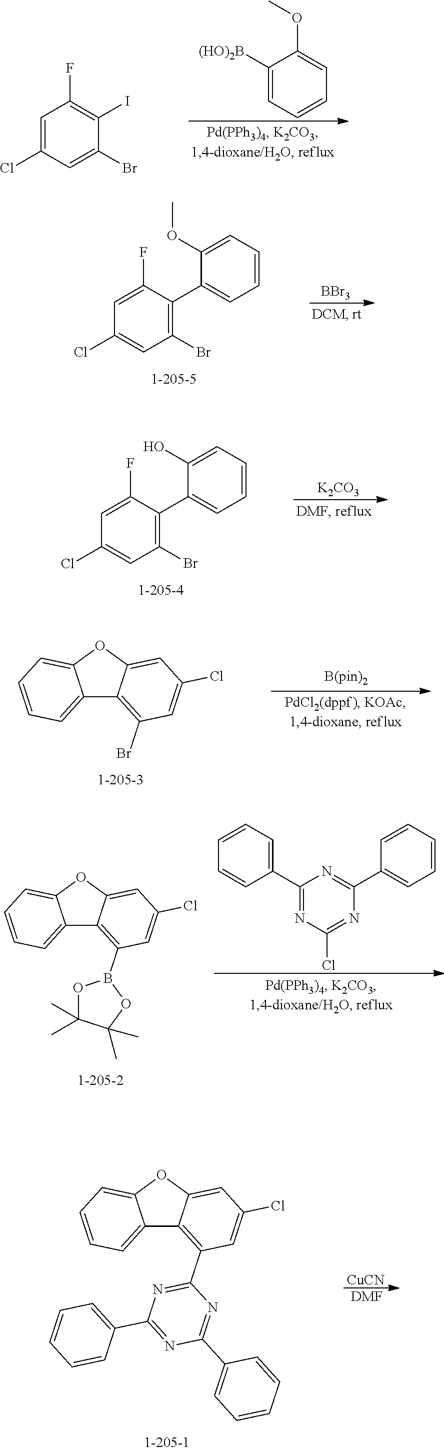

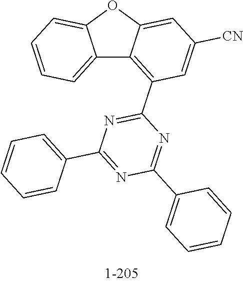 [SMILES: N#Cc1cc(-c2nc(-c3ccccc3)nc(-c3ccccc3)n2)c2c(c1)oc1ccccc12]

1) Preparation of Compound 1-205

After dissolving Compound 1-205-1 (30.0 g, 131.8 mM) and copper(I) cyanide (23.6 g, 263.6 mM) in DMF (300 mL), the result was refluxed for 24 hours. After the reaction was completed, the copper(I) cyanide was filtered, and the result was extracted by introducing distilled water and DCM thereto at room temperature. After drying the organic layer with MgSO₄, the solvent was removed using a rotary evaporator. The reaction material was purified using column chromatography (DCM:Hex=1:3), and recrystallized with methanol to obtain target Compound 1-205 (24.9 g, 83%).

Target Compound A was synthesized in the same manner as in the preparation of Preparation Example 6 except that Intermediate A of the following Table 6 was used instead of 2-chloro-4,6-diphenyl-1,3,5-triazine, and Intermediate B of the following Table 6 was used instead of copper(I) cyanide.

TABLE 6

| Compound | | | | |
|---|---|---|---|---|
| No. | Intermediate A | Intermediate B | Target Compound A | Yield |
| 1-208 | 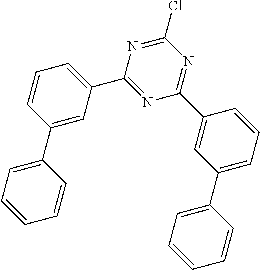 [SMILES: Clc1nc(-c2cccc(-c3ccccc3)c2)nc(-c2cccc(-c3ccccc3)c2)n1] | CuCN | 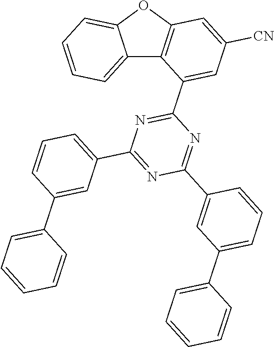 [SMILES: N#Cc1cc(-c2nc(-c3cccc(-c4ccccc4)c3)nc(-c3cccc(-c4ccccc4)c3)n2)c2c(c1)oc1ccccc12] | 33% |

### <Preparation Example 7> Preparation of Compound 2-1-3

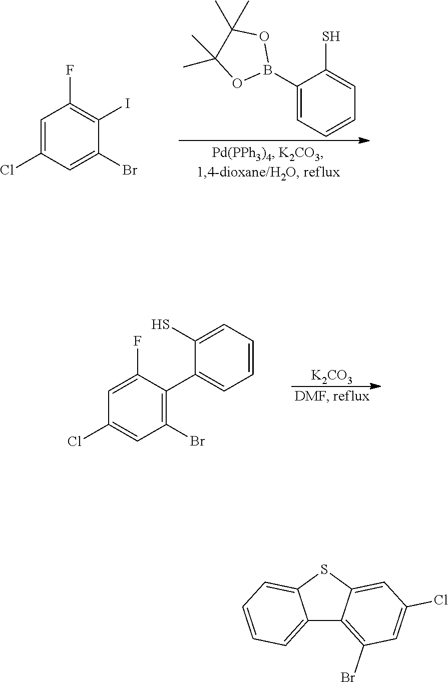

1) Preparation of Compound 2-1-4

After dissolving 1-bromo-5-chloro-3-fluoro-2-iodobenzene (20.0 g, 59.6 mM), 2-(4,4,5,5-tetramethyl-1,3,2-dioxaborolan-2-yl)benzenethiol (12.8 g, 54.2 mM), Pd(PPh)₄ (3.1 g, 2.7 mM) and K₂CO₃ (15.0 g, 108.4 mM) in 1,4-dioxane/H₂O (200 mL/40 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:10) to obtain target Compound 2-1-4 (13.8 g, 80%).

2) Preparation of Compound 2-1-3

After dissolving Compound 2-1-4 (6.9 g, 21.6 mM) and K₂CO₃ (59.9 g, 43.3 mM) in DMF (60 mL), the result was refluxed for 4 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:5), and recrystallized with methanol to obtain target Compound 2-1-3 (5.8 g, 90%).

Target Compound A was synthesized in the same manner as in the preparation of Preparation Example 1 except that Compound 2-1-3 was used instead of Compound 1-1-3, Intermediate A of the following Table 7 was used instead of (9-phenyl-9H-carbazol-3-yl)boronic acid, and Intermediate B of the following Table 7 was used instead of 2-chloro-4,6-diphenyl-1,3,5-triazine.

TABLE 7

| Com- | | | | |
|---|---|---|---|---|
| pound | | | | |
| No. | Intermediate A | Intermediate B | Target Compound A | Yield |
| 2-1 | 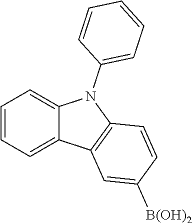 [SMILES: OB(O)c1ccc2c(c1)c1ccccc1n2-c1ccccc1] | 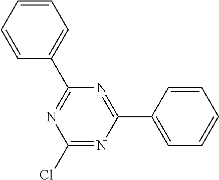 [SMILES: Clc1nc(-c2ccccc2)nc(-c2ccccc2)n1] | 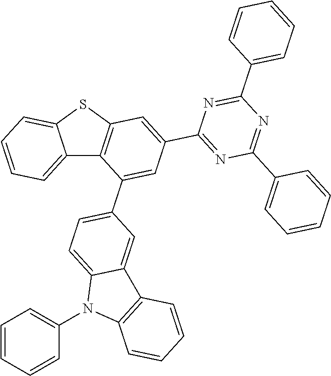 [SMILES: c1ccc(-c2nc(-c3ccccc3)nc(-c3cc(-c4ccc5c(c4)c4ccccc4n5-c4ccccc4)c4c(c3)sc3ccccc34)n2)cc1] | 34% |
| 2-2 | | 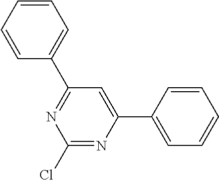 [SMILES: Clc1nc(-c2ccccc2)cc(-c2ccccc2)n1] | 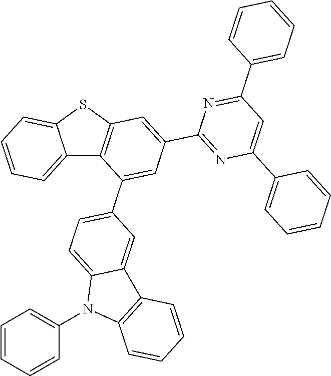 [SMILES: c1ccc(-c2cc(-c3ccccc3)nc(-c3cc(-c4ccc5c(c4)c4ccccc4n5-c4ccccc4)c4c(c3)sc3ccccc34)n2)cc1] | 35% |
| 2-9 | | 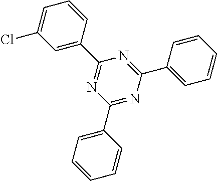 [SMILES: Clc1cccc(-c2nc(-c3ccccc3)nc(-c3ccccc3)n2)c1] | 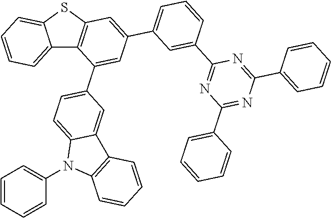 [SMILES: c1ccc(-c2nc(-c3ccccc3)nc(-c3cccc(-c4cc(-c5ccc6c(c5)c5ccccc5n6-c5ccccc5)c5c(c4)sc4ccccc45)c3)n2)cc1] | 36% |
| 2-26 | 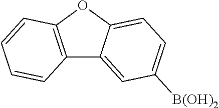 [SMILES: OB(O)c1ccc2oc3ccccc3c2c1] | 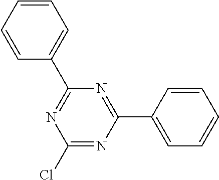 [SMILES: Clc1nc(-c2ccccc2)nc(-c2ccccc2)n1] | 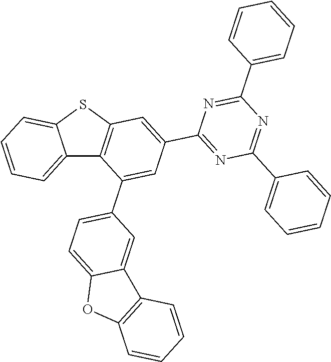 [SMILES: c1ccc(-c2nc(-c3ccccc3)nc(-c3cc(-c4ccc5oc6ccccc6c5c4)c4c(c3)sc3ccccc34)n2)cc1] | 32% |
| 2-40 | 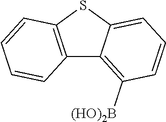 [SMILES: OB(O)c1cccc2sc3ccccc3c12] | 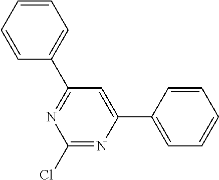 [SMILES: Clc1nc(-c2ccccc2)cc(-c2ccccc2)n1] | 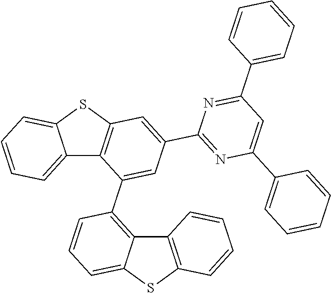 [SMILES: c1ccc(-c2cc(-c3ccccc3)nc(-c3cc(-c4cccc5sc6ccccc6c45)c4c(c3)sc3ccccc34)n2)cc1] | 32% |
| 2-41 | 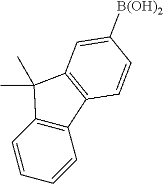 [SMILES: CC1(C)c2ccccc2-c2ccc(B(O)O)cc21] | 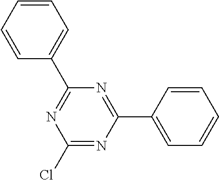 [SMILES: Clc1nc(-c2ccccc2)nc(-c2ccccc2)n1] | 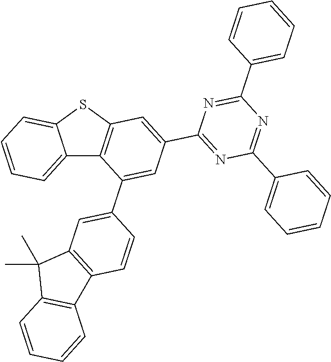 [SMILES: CC1(C)c2ccccc2-c2ccc(-c3cc(-c4nc(-c5ccccc5)nc(-c5ccccc5)n4)cc4sc5ccccc5c34)cc21] | 33% |
| 2-50 | 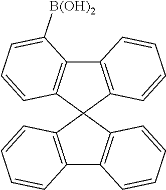 [SMILES: OB(O)c1cccc2c1-c1ccccc1C21c2ccccc2-c2ccccc21] | | 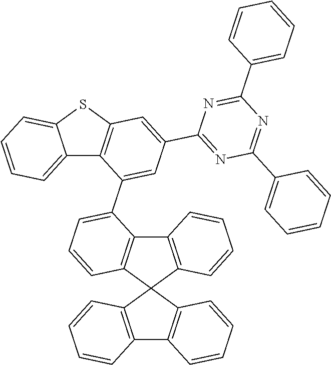 [SMILES: c1ccc(-c2nc(-c3ccccc3)nc(-c3cc(-c4cccc5c4-c4ccccc4C54c5ccccc5-c5ccccc54)c4c(c3)sc3ccccc34)n2)cc1] | 35% |

Target Compound A was synthesized in the same manner as in the preparation of Preparation Example 2 except that Compound 2-1-3 of Preparation Example 7 was used instead of Compound 1-121-3, Intermediate A of the following Table 8 was used instead of di([1,1′-biphenyl]-4-yl)amine, and Intermediate B of the following Table 8 was used instead of 2-chloro-4,6-diphenyl-1,3,5-triazine.

TABLE 8

| Com- | | | | |
|---|---|---|---|---|
| pound | | | | |
| No. | Intermediate A | Intermediate B | Target Compound A | Yield |
| 2-51 | 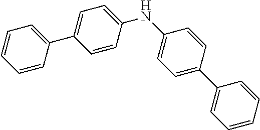 [SMILES: c1ccc(-c2ccc(Nc3ccc(-c4ccccc4)cc3)cc2)cc1] | 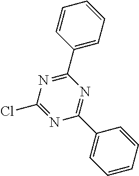 [SMILES: Clc1nc(-c2ccccc2)nc(-c2ccccc2)n1] | 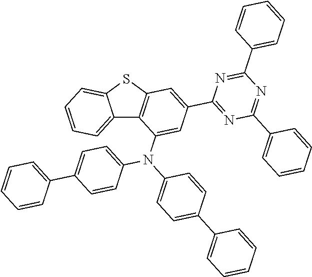 [SMILES: c1ccc(-c2ccc(N(c3ccc(-c4ccccc4)cc3)c3cc(-c4nc(-c5ccccc5)nc(-c5ccccc5)n4)cc4sc5ccccc5c34)cc2)cc1] | 35% |

Target Compound A was synthesized in the same manner as in the preparation of Preparation Example 3 except that Compound 2-1-3 of Preparation Example 7 was used instead of Compound 1-172-4, Intermediate A of the following Table 9 was used instead of N-([1,1′-biphenyl]-4-yl)-N-(4-bromophenyl)-[1,1′-biphenyl]-4-amine, and Intermediate B of the following Table 9 was used instead of 2-chloro-4,6-diphenyl-1,3,5-triazine.

TABLE 9

| Com- | | | | |
|---|---|---|---|---|
| pound | | | | |
| No. | Intermediate A | Intermediate B | Target Compound A | Yield |
| 2-56 | 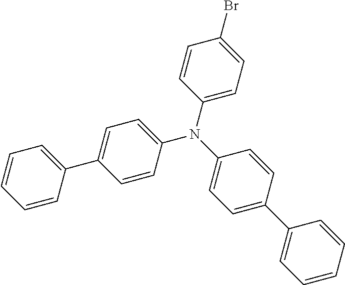 [SMILES: Brc1ccc(N(c2ccc(-c3ccccc3)cc2)c2ccc(-c3ccccc3)cc2)cc1] | 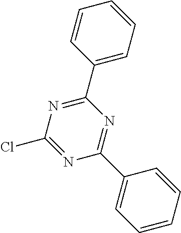 [SMILES: Clc1nc(-c2ccccc2)nc(-c2ccccc2)n1] | 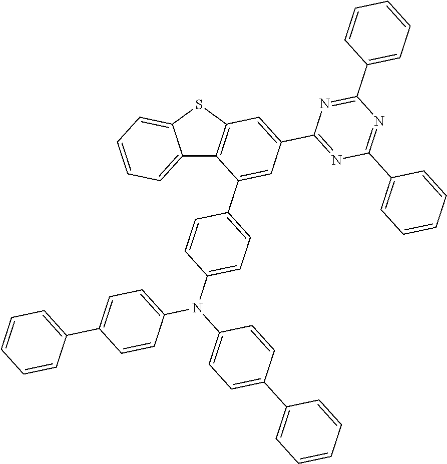 [SMILES: c1ccc(-c2ccc(N(c3ccc(-c4ccccc4)cc3)c3ccc(-c4cc(-c5nc(-c6ccccc6)nc(-c6ccccc6)n5)cc5sc6ccccc6c45)cc3)cc2)cc1] | 32% |
| 2-60 | 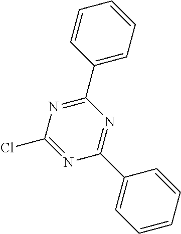 [SMILES: Clc1nc(-c2ccccc2)nc(-c2ccccc2)n1] | 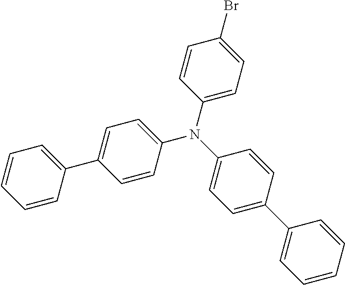 [SMILES: Brc1ccc(N(c2ccc(-c3ccccc3)cc2)c2ccc(-c3ccccc3)cc2)cc1] | 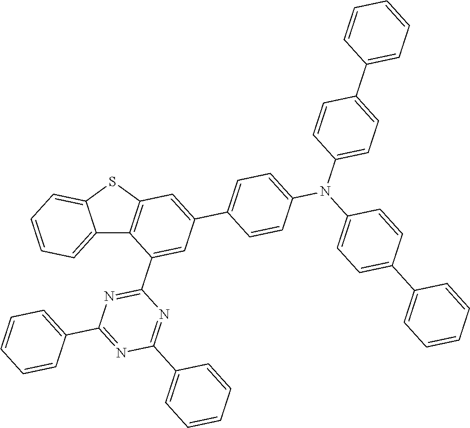 [SMILES: c1ccc(-c2ccc(N(c3ccc(-c4ccccc4)cc3)c3ccc(-c4cc(-c5nc(-c6ccccc6)nc(-c6ccccc6)n5)c5c(c4)sc4ccccc45)cc3)cc2)cc1] | 34% |
| 2-63 | 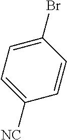 | 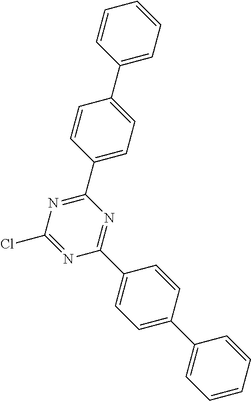 [SMILES: Clc1nc(-c2ccc(-c3ccccc3)cc2)nc(-c2ccc(-c3ccccc3)cc2)n1] | 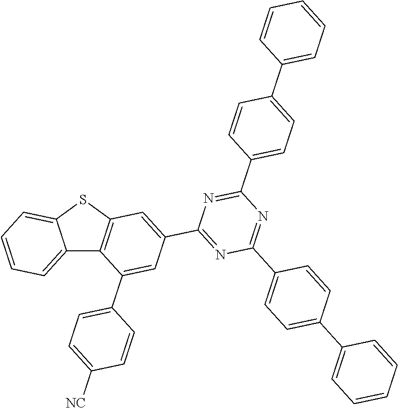 [SMILES: N#Cc1ccc(-c2cc(-c3nc(-c4ccc(-c5ccccc5)cc4)nc(-c4ccc(-c5ccccc5)cc4)n3)cc3sc4ccccc4c23)cc1] | 36% |

Target Compound A was synthesized in the same manner as in the preparation of Preparation Example 4 except that Compound 2-1-3 of Preparation Example 7 was used instead of Compound 1-178-3, Intermediate A of the following Table 10 was used instead of 2-chloro-4,6-diphenyl-1,3,5-triazine, and Intermediate B of the following Table 10 was used instead of di([1,1′-biphenyl]-4-yl)amine.

TABLE 10

| Compound | | | | |
|---|---|---|---|---|
| No. | Intermediate A | Intermediate B | Target Compound A | Yield |
| 2-57 | 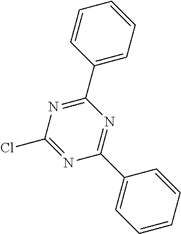 [SMILES: Clc1nc(-c2ccccc2)nc(-c2ccccc2)n1] | 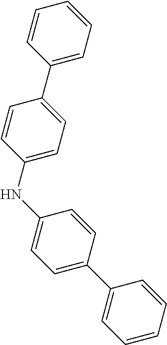 [SMILES: c1ccc(-c2ccc(Nc3ccc(-c4ccccc4)cc3)cc2)cc1] | 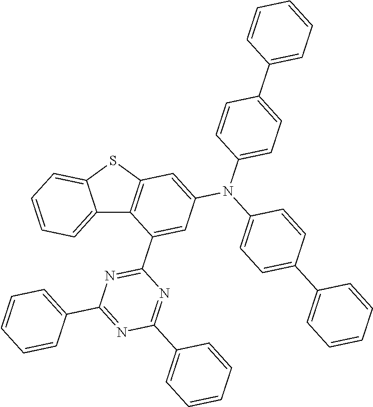 [SMILES: c1ccc(-c2ccc(N(c3ccc(-c4ccccc4)cc3)c3cc(-c4nc(-c5ccccc5)nc(-c5ccccc5)n4)c4c(c3)sc3ccccc34)cc2)cc1] | 33% |

Target Compound A was synthesized in the same manner as in the preparation of Preparation Example 5 except that Compound 2-1-3 of Preparation Example 7 was used instead of Compound 1-197-3, Intermediate A of the following Table 11 was used instead of copper(I) cyanide, and Intermediate B of the following Table 11 was used instead of 2,4-di([1,1′-biphenyl]-4-yl)-6-chloro-1,3,5-triazine.

TABLE 11

| Com- | | | | |
|---|---|---|---|---|
| pound | | | | |
| No. | Intermediate A | Intermediate B | Target Compound A | Yield |
| 2-61 | CuCN | 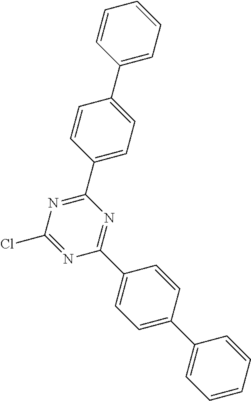 [SMILES: Clc1nc(-c2ccc(-c3ccccc3)cc2)nc(-c2ccc(-c3ccccc3)cc2)n1] | 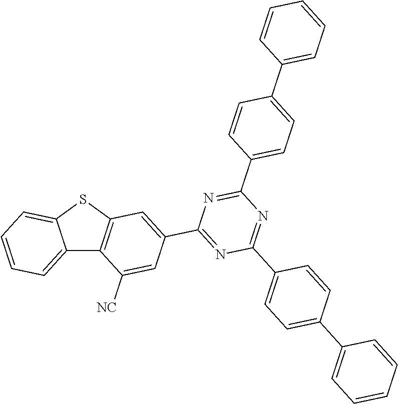 [SMILES: N#Cc1cc(-c2nc(-c3ccc(-c4ccccc4)cc3)nc(-c3ccc(-c4ccccc4)cc3)n2)cc2sc3ccccc3c12] | 34% |

Target Compound A was synthesized in the same manner as in the preparation of Preparation Example 6 except that Compound 2-1-3 of Preparation Example 7 was used instead of Compound 1-205-3, Intermediate A of the following Table 12 was used instead of 2-chloro-4,6-diphenyl-1,3,5-triazine, and Intermediate B of the following Table 12 was used instead of copper (I) cyanide.

TABLE 12

| Com- | | | | |
|---|---|---|---|---|
| pound | | | | |
| No. | Intermediate A | Intermediate B | Target Compound A | Yield |
| 2-64 | 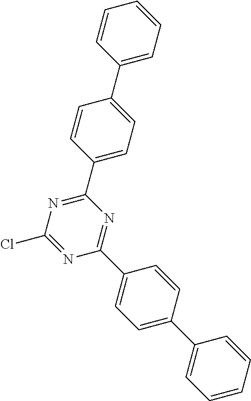 [SMILES: Clc1nc(-c2ccc(-c3ccccc3)cc2)nc(-c2ccc(-c3ccccc3)cc2)n1] | CuCN | 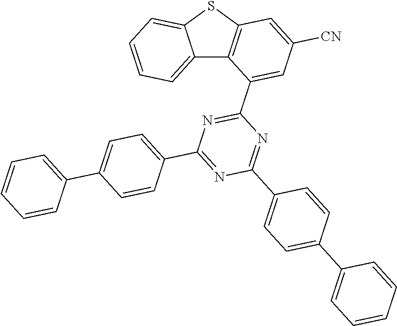 [SMILES: N#Cc1cc(-c2nc(-c3ccc(-c4ccccc4)cc3)nc(-c3ccc(-c4ccccc4)cc3)n2)c2c(c1)sc1ccccc12] | 33% |

### <Preparation Example 8> Preparation of Compound 3-1-3

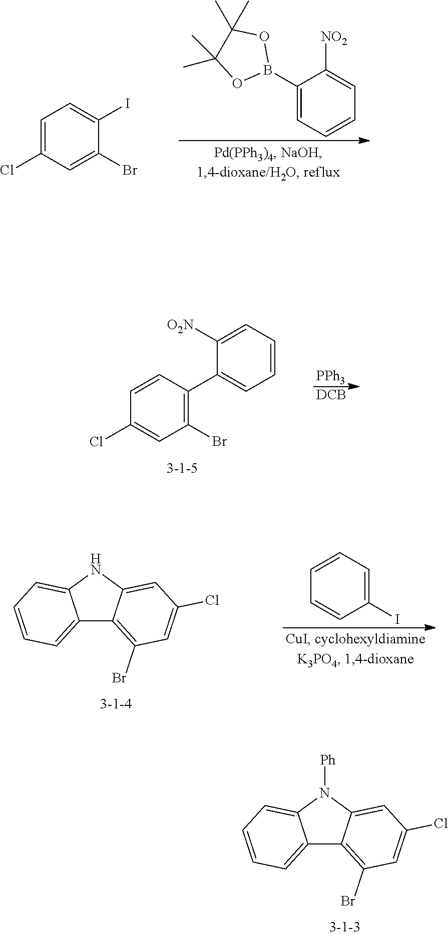

1) Preparation of Compound 3-1-5

After dissolving 2-bromo-4-chloro-1-iodobenzene (20.0 g, 63.0 mM), 4,4,5,5-tetramethyl-2-(2-nitrophenyl)-1,3,2-dioxaborolane (15.7 g, 63.0 mM), Pd(PPh)₄ (3.6 g, 3.2 mM) and K₂CO₃ (17.4 g, 126.0 mM) in 1,4-dioxane/H₂O (200 mL/40 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 3-1-5 (16.7 g, 85%).

2) Preparation of Compound 3-1-4

After dissolving Compound 3-1-5 (16.0 g, 51.2 mM) and PPh₃ (33.6 g, 128.0 mM) in DCB (130 mL), the result was refluxed for 24 hours. After the reaction was completed, DCB was removed using a rotary evaporator, and the result was extracted by introducing distilled water and DCM thereto at room temperature. After drying the organic layer with MgSO₄, the solvent was removed using a rotary evaporator. The reaction material was purified using column chromatography (DCM:Hex=1:10) to obtain target Compound 3-1-4 (11.5 g, 80%).

3) Preparation of Compound 3-1-3

After dissolving Compound 3-1-4 (11.0 g, 39.2 mM), iodobenzene (11.0 mL, 98.0 mM), CuI (7.5 g, 39.2 mM), trans-1,2-diaminocyclohexane (5.2 mL, 39.2 mM) and K₃PO₄ (16.6 g, 78.4 mM) in 1,4-dioxane (100 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:6), and recrystallized with methanol to obtain target Compound 3-1-3 (12.0 g, 86%).

Target Compound A was synthesized in the same manner as in the preparation of Preparation Example 1 except that Compound 3-1-3 of Preparation Example 8 was used instead of Compound 1-1-3, Intermediate A of the following Table 13 was used instead of (9-phenyl-9H-carbazol-3-yl)boronic acid, and Intermediate B of the following Table 13 was used instead of 2-chloro-4,6-diphenyl-1,3,5-triazine.

TABLE 13

| Compound | | | | Total |
|---|---|---|---|---|
| No. | Intermediate A | Intermediate B | Target Compound A | Yield |
| 3-1 | 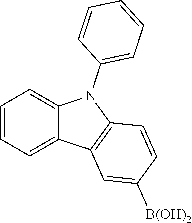 [SMILES: OB(O)c1ccc2c(c1)c1ccccc1n2-c1ccccc1] | 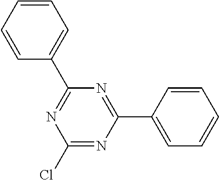 [SMILES: Clc1nc(-c2ccccc2)nc(-c2ccccc2)n1] | 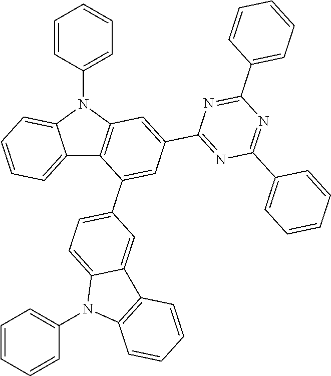 [SMILES: c1ccc(-c2nc(-c3ccccc3)nc(-c3cc(-c4ccc5c(c4)c4ccccc4n5-c4ccccc4)c4c5ccccc5n(-c5ccccc5)c4c3)n2)cc1] | 38% |
| 3-2 | | 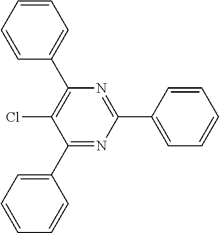 [SMILES: Clc1c(-c2ccccc2)nc(-c2ccccc2)nc1-c1ccccc1] | 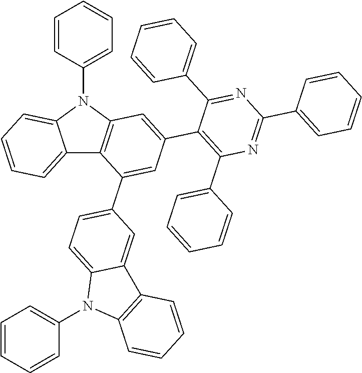 [SMILES: c1ccc(-c2nc(-c3ccccc3)c(-c3cc(-c4ccc5c(c4)c4ccccc4n5-c4ccccc4)c4c5ccccc5n(-c5ccccc5)c4c3)c(-c3ccccc3)n2)cc1] | 33% |
| 3-7 | | 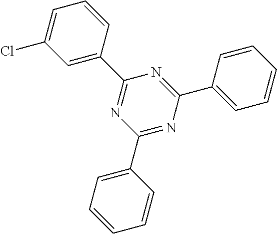 [SMILES: Clc1cccc(-c2nc(-c3ccccc3)nc(-c3ccccc3)n2)c1] | 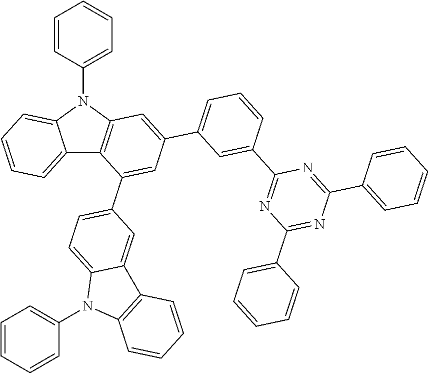 [SMILES: c1ccc(-c2nc(-c3ccccc3)nc(-c3cccc(-c4cc(-c5ccc6c(c5)c5ccccc5n6-c5ccccc5)c5c6ccccc6n(-c6ccccc6)c5c4)c3)n2)cc1] | 34% |
| 3-9 | | 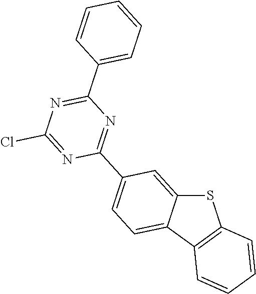 [SMILES: Clc1nc(-c2ccccc2)nc(-c2ccc3c(c2)sc2ccccc23)n1] | 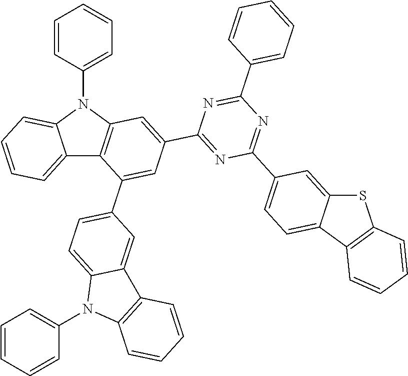 [SMILES: c1ccc(-c2nc(-c3ccc4c(c3)sc3ccccc34)nc(-c3cc(-c4ccc5c(c4)c4ccccc4n5-c4ccccc4)c4c5ccccc5n(-c5ccccc5)c4c3)n2)cc1] | 34% |
| 3-11 | | 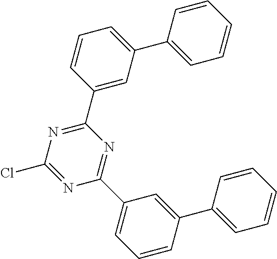 [SMILES: Clc1nc(-c2cccc(-c3ccccc3)c2)nc(-c2cccc(-c3ccccc3)c2)n1] | 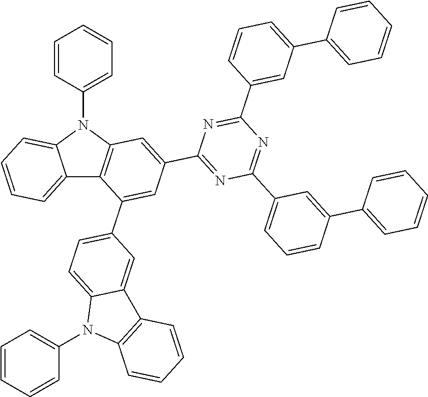 [SMILES: c1ccc(-c2cccc(-c3nc(-c4cccc(-c5ccccc5)c4)nc(-c4cc(-c5ccc6c(c5)c5ccccc5n6-c5ccccc5)c5c6ccccc6n(-c6ccccc6)c5c4)n3)c2)cc1] | 36% |
| 3-21 | 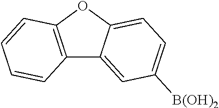 [SMILES: OB(O)c1ccc2oc3ccccc3c2c1] | 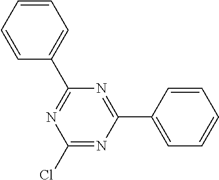 [SMILES: Clc1nc(-c2ccccc2)nc(-c2ccccc2)n1] | 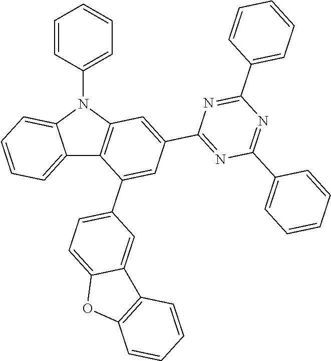 [SMILES: c1ccc(-c2nc(-c3ccccc3)nc(-c3cc(-c4ccc5oc6ccccc6c5c4)c4c5ccccc5n(-c5ccccc5)c4c3)n2)cc1] | 35% |
| 3-39 | 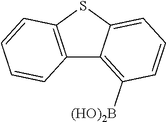 [SMILES: OB(O)c1cccc2sc3ccccc3c12] | 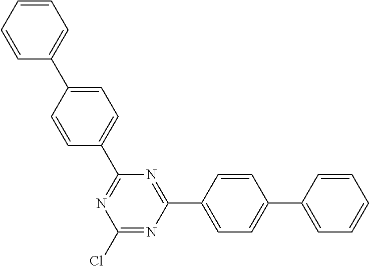 [SMILES: Clc1nc(-c2ccc(-c3ccccc3)cc2)nc(-c2ccc(-c3ccccc3)cc2)n1] | 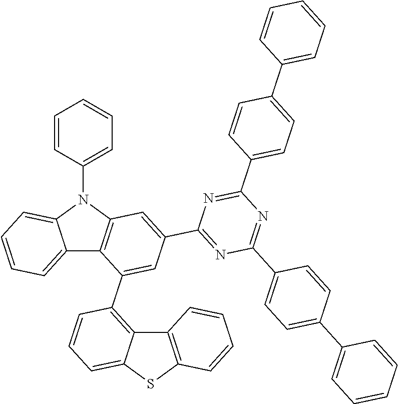 [SMILES: c1ccc(-c2ccc(-c3nc(-c4ccc(-c5ccccc5)cc4)nc(-c4cc(-c5cccc6sc7ccccc7c56)c5c6ccccc6n(-c6ccccc6)c5c4)n3)cc2)cc1] | 37% |
| 3-41 | 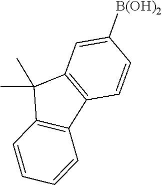 [SMILES: CC1(C)c2ccccc2-c2ccc(B(O)O)cc21] | 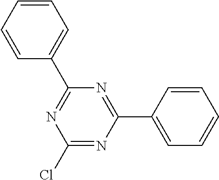 [SMILES: Clc1nc(-c2ccccc2)nc(-c2ccccc2)n1] | 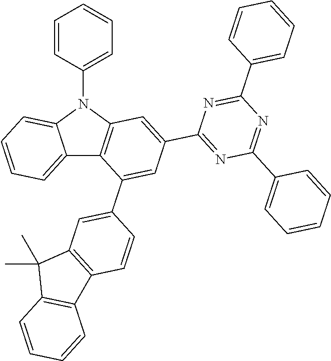 [SMILES: CC1(C)c2ccccc2-c2ccc(-c3cc(-c4nc(-c5ccccc5)nc(-c5ccccc5)n4)cc4c3c3ccccc3n4-c3ccccc3)cc21] | 33% |
| 3-49 | 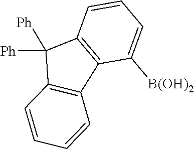 [SMILES: OB(O)c1cccc2c1-c1ccccc1C2(c1ccccc1)c1ccccc1] | | 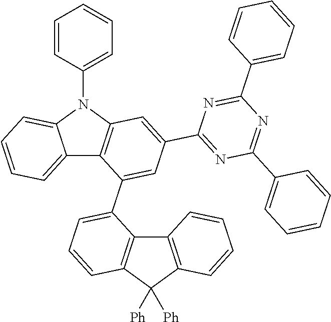 [SMILES: c1ccc(-c2nc(-c3ccccc3)nc(-c3cc(-c4cccc5c4-c4ccccc4C5(c4ccccc4)c4ccccc4)c4c5ccccc5n(-c5ccccc5)c4c3)n2)cc1] | 36% |

Target Compound A was synthesized in the same manner as in the preparation of Preparation Example 2 except that Compound 3-1-3 of Preparation Example 8 was used instead of Compound 1-121-3, Intermediate A of the following Table 14 was used instead of di([1,1′-biphenyl]-4-yl)amine, and Intermediate B of the following Table 14 was used instead of 2-chloro-4,6-diphenyl-1,3,5-triazine.

TABLE 14

| Com- | | | | |
|---|---|---|---|---|
| pound | | | | |
| No. | Intermediate A | Intermediate B | Target Compound A | Yield |
| 3-51 | 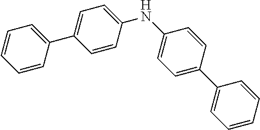 [SMILES: c1ccc(-c2ccc(Nc3ccc(-c4ccccc4)cc3)cc2)cc1] | 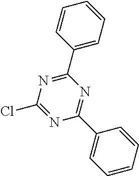 [SMILES: Clc1nc(-c2ccccc2)nc(-c2ccccc2)n1] | 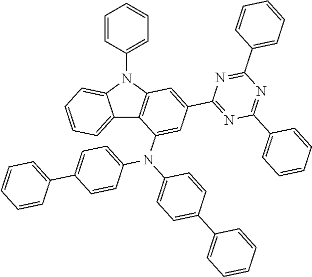 [SMILES: c1ccc(-c2ccc(N(c3ccc(-c4ccccc4)cc3)c3cc(-c4nc(-c5ccccc5)nc(-c5ccccc5)n4)cc4c3c3ccccc3n4-c3ccccc3)cc2)cc1] | 35% |

Target Compound A was synthesized in the same manner as in the preparation of Preparation Example 3 except that Compound 3-1-3 of Preparation Example 8 was used instead of Compound 1-172-4, Intermediate A of the following Table 15 was used instead of N-([1,1′-biphenyl]-4-yl)-N-(4-bromophenyl)-[1,1′-biphenyl]-4-amine, and Intermediate B of the following Table 15 was used instead of 2-chloro-4,6-diphenyl-1,3,5-triazine.

TABLE 15

| Com- | | | | |
|---|---|---|---|---|
| pound | | | | |
| No. | Intermediate A | Intermediate B | Target Compound A | Yield |
| 3-56 | 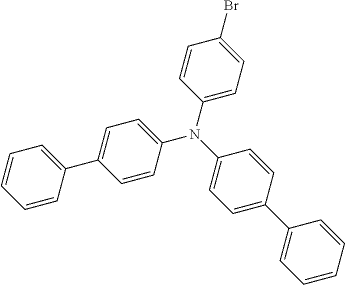 [SMILES: Brc1ccc(N(c2ccc(-c3ccccc3)cc2)c2ccc(-c3ccccc3)cc2)cc1] | 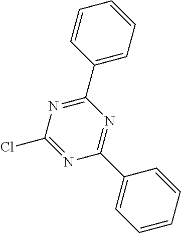 [SMILES: Clc1nc(-c2ccccc2)nc(-c2ccccc2)n1] | 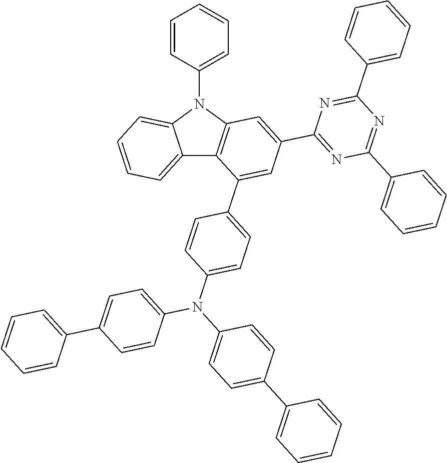 [SMILES: c1ccc(-c2ccc(N(c3ccc(-c4ccccc4)cc3)c3ccc(-c4cc(-c5nc(-c6ccccc6)nc(-c6ccccc6)n5)cc5c4c4ccccc4n5-c4ccccc4)cc3)cc2)cc1] | 35% |
| 3-60 | 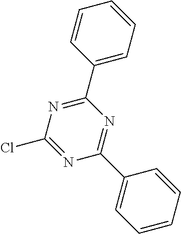 [SMILES: Clc1nc(-c2ccccc2)nc(-c2ccccc2)n1] | 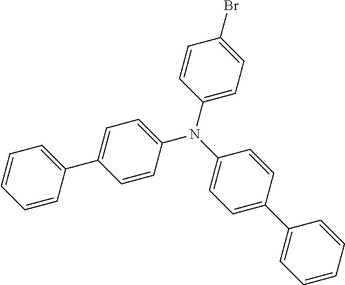 [SMILES: Brc1ccc(N(c2ccc(-c3ccccc3)cc2)c2ccc(-c3ccccc3)cc2)cc1] | 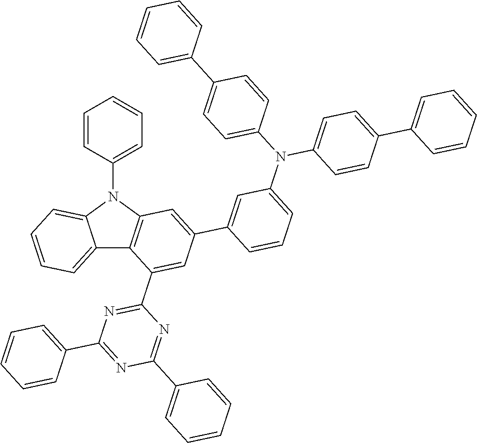 [SMILES: c1ccc(-c2ccc(N(c3ccc(-c4ccccc4)cc3)c3cccc(-c4cc(-c5nc(-c6ccccc6)nc(-c6ccccc6)n5)c5c6ccccc6n(-c6ccccc6)c5c4)c3)cc2)cc1] | 34% |
| 3-63 | 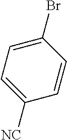 | 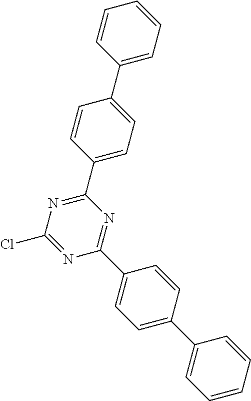 [SMILES: Clc1nc(-c2ccc(-c3ccccc3)cc2)nc(-c2ccc(-c3ccccc3)cc2)n1] | 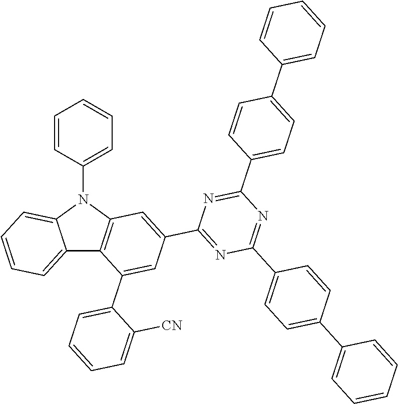 [SMILES: N#Cc1ccccc1-c1cc(-c2nc(-c3ccc(-c4ccccc4)cc3)nc(-c3ccc(-c4ccccc4)cc3)n2)cc2c1c1ccccc1n2-c1ccccc1] | 32% |

Target Compound A was synthesized in the same manner as in the preparation of Preparation Example 4 except that Compound 3-1-3 of Preparation Example 8 was used instead of Compound 1-178-3, Intermediate A of the following Table 16 was used instead of 2-chloro-4,6-diphenyl-1,3,5-triazine, and Intermediate B of the following Table 16 was used instead of di([1,1′-biphenyl]-4-yl)amine.

TABLE 16

| Compound | | | | |
|---|---|---|---|---|
| No. | Intermediate A | Intermediate B | Target Compound A | Yield |
| 3-57 | 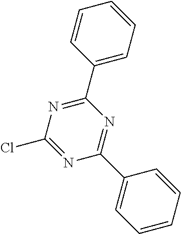 [SMILES: Clc1nc(-c2ccccc2)nc(-c2ccccc2)n1] | 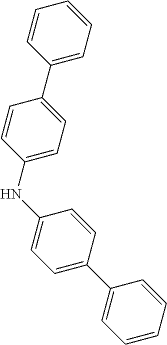 [SMILES: c1ccc(-c2ccc(Nc3ccc(-c4ccccc4)cc3)cc2)cc1] | 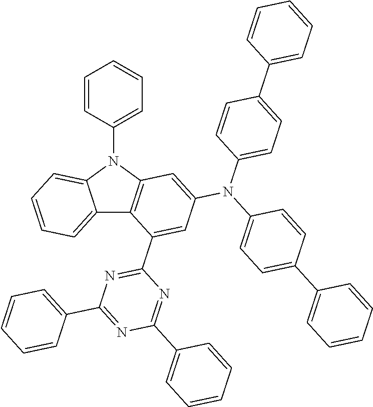 [SMILES: c1ccc(-c2ccc(N(c3ccc(-c4ccccc4)cc3)c3cc(-c4nc(-c5ccccc5)nc(-c5ccccc5)n4)c4c5ccccc5n(-c5ccccc5)c4c3)cc2)cc1] | 34% |

Target Compound A was synthesized in the same manner as in the preparation of Preparation Example 5 except that Compound 3-1-3 of Preparation Example 8 was used instead of Compound 1-197-3, Intermediate A of the following Table 17 was used instead of copper(I) cyanide, and Intermediate B of the following Table 17 was used instead of 2,4-di([1,1′-biphenyl]-4-yl)-6-chloro-1,3,5-triazine. PGP-A

TABLE 17

| Com- | | | | |
|---|---|---|---|---|
| pound | | | | |
| No. | Intermediate A | Intermediate B | Target Compound A | Yield |
| 3-61 | CuCN | 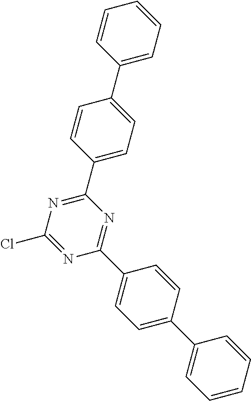 [SMILES: Clc1nc(-c2ccc(-c3ccccc3)cc2)nc(-c2ccc(-c3ccccc3)cc2)n1] | 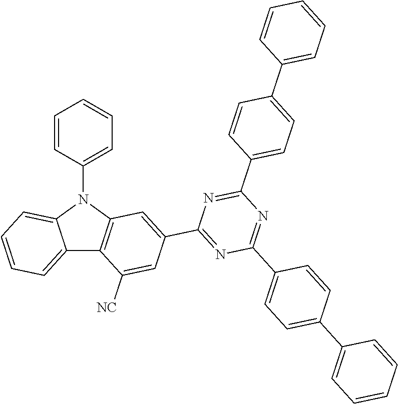 [SMILES: N#Cc1cc(-c2nc(-c3ccc(-c4ccccc4)cc3)nc(-c3ccc(-c4ccccc4)cc3)n2)cc2c1c1ccccc1n2-c1ccccc1] | 35% |

Target Compound A was synthesized in the same manner as in the preparation of Preparation Example 6 except that Compound 3-1-3 of Preparation Example 8 was used instead of Compound 1-205-3, Intermediate A of the following Table 18 was used instead of 2-chloro-4,6-diphenyl-1,3,5-triazine, and Intermediate B of the following Table 18 was used instead of copper (I) cyanide.

TABLE 18

| Com- | | | | |
|---|---|---|---|---|
| pound | | | | |
| No. | Intermediate A | Intermediate B | target Compound A | Yield |
| 3-64 | 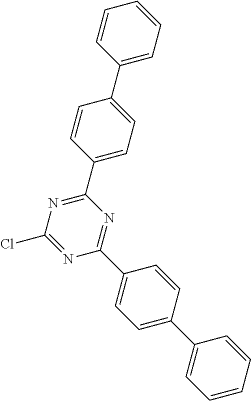 [SMILES: Clc1nc(-c2ccc(-c3ccccc3)cc2)nc(-c2ccc(-c3ccccc3)cc2)n1] | CuCN | 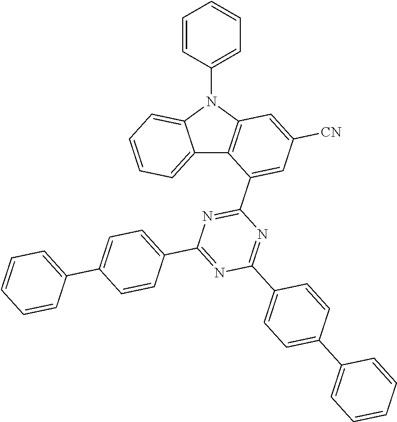 [SMILES: N#Cc1cc(-c2nc(-c3ccc(-c4ccccc4)cc3)nc(-c3ccc(-c4ccccc4)cc3)n2)c2c3ccccc3n(-c3ccccc3)c2c1] | 33% |

### <Preparation Example 9> Synthesis of Compound 4-3

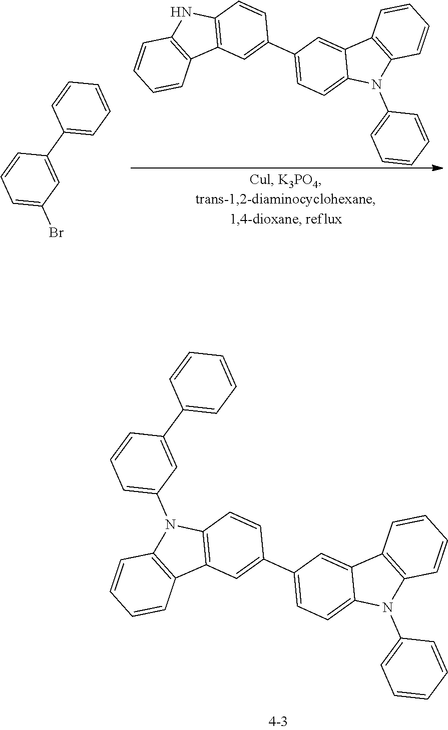

1) Preparation of Compound 4-3

After dissolving 3-bromo-1,1′-biphenyl (3.7 g, 15.8 mM), 9-phenyl-9H,9′H-3,3′-bicarbazole (6.5 g, 15.8 mM), CuI (3.0 g, 15.8 mM), trans-1,2-diaminocyclohexane (1.9 mL, 15.8 mM) and K₃PO₄ (3.3 g, 31.6 mM) in 1,4-oxane (100 mL), the result was refluxed for 24 hours. After the reaction was completed, the result was extracted by introducing distilled water and DCM thereto at room temperature, and after drying the organic layer with MgSO₄, the solvent was removed using a rotary evaporator. The reaction material was purified using column chromatography (DCM:Hex=1:3), and recrystallized with methanol to obtain target Compound 4-3 (7.5 g, 85%).

Target Compound A was synthesized in the same manner as in the preparation of Preparation Example 9 except that Intermediate A of the following Table 19 was used instead of 3-bromo-1,1′-biphenyl, and Intermediate B of the following Table 19 was used instead of 9-phenyl-9H, 9′H-3,3′-bicarbazole.

TABLE 19

| Com- | | | | |
|---|---|---|---|---|
| pound | | | | Total |
| No. | Intermediate A | Intermediate B | Target Compound A | Yield |
| 4-4 | 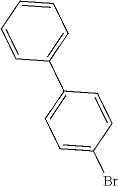 [SMILES: Brc1ccc(-c2ccccc2)cc1] | 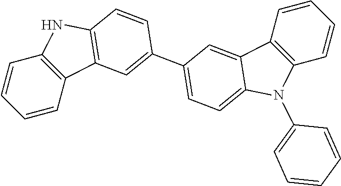 [SMILES: c1ccc(-n2c3ccccc3c3cc(-c4ccc5[nH]c6ccccc6c5c4)ccc32)cc1] | 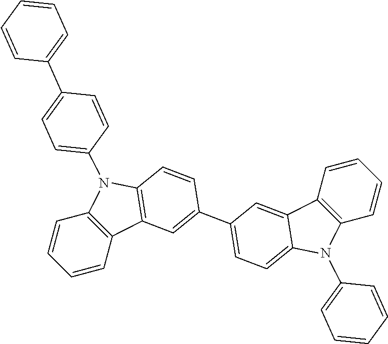 [SMILES: c1ccc(-c2ccc(-n3c4ccccc4c4cc(-c5ccc6c(c5)c5ccccc5n6-c5ccccc5)ccc43)cc2)cc1] | 83% |
| 4-7 | 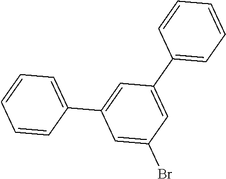 [SMILES: Brc1cc(-c2ccccc2)cc(-c2ccccc2)c1] | | 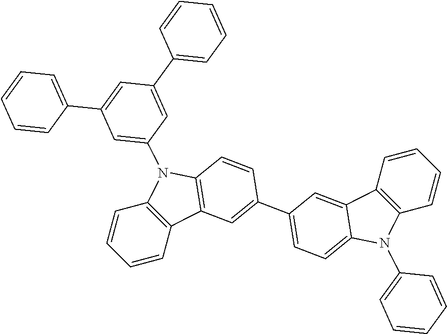 [SMILES: c1ccc(-c2cc(-c3ccccc3)cc(-n3c4ccccc4c4cc(-c5ccc6c(c5)c5ccccc5n6-c5ccccc5)ccc43)c2)cc1] | 84% |
| 4-31 | 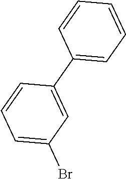 [SMILES: Brc1cccc(-c2ccccc2)c1] | 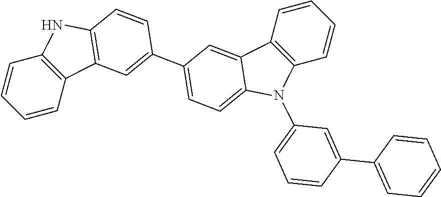 [SMILES: c1ccc(-c2cccc(-n3c4ccccc4c4cc(-c5ccc6[nH]c7ccccc7c6c5)ccc43)c2)cc1] | 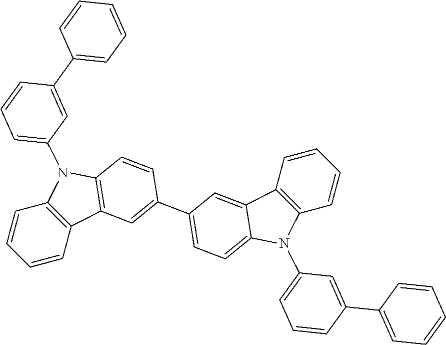 [SMILES: c1ccc(-c2cccc(-n3c4ccccc4c4cc(-c5ccc6c(c5)c5ccccc5n6-c5cccc(-c6ccccc6)c5)ccc43)c2)cc1] | 81% |
| 4-32 | 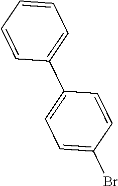 [SMILES: Brc1ccc(-c2ccccc2)cc1] | | 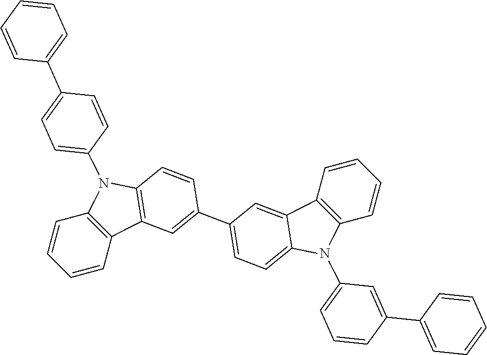 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 80% |
| 4-42 | 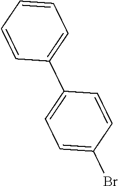 [SMILES: Brc1ccc(-c2ccccc2)cc1] | 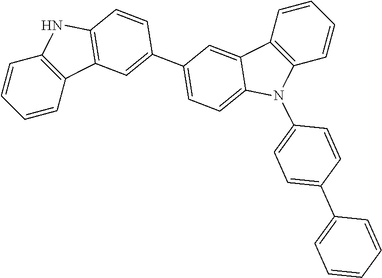 [SMILES: c1ccc(-c2ccc(-n3c4ccccc4c4cc(-c5ccc6[nH]c7ccccc7c6c5)ccc43)cc2)cc1] | 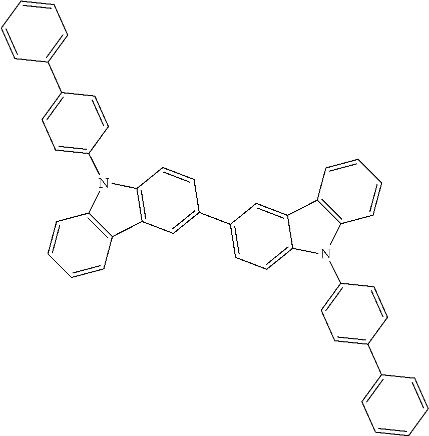 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 82% |

Compounds other than the compounds described in Table 1 to Table 19 were also prepared in the same manner as in the methods described in the preparation examples provided above.

The following Table 20 and Table 21 present 1H NMR data and FD-MS data of the synthesized compounds, and through the following data, syntheses of target compounds may be identified.

TABLE 20

| Compound | |
|---|---|
| No. | ¹H NMR (CDCl₃, 200 Mz) |
| 1-1 | δ = 8.28(4H, d), 8.18(1H, d), 8.12(1H, d), 8.00(1H, d), |
| | 7.89(1H, d), 7.77(1H, s), 7.41-7.66(19H, m) |
| 1-2 | δ = 8.23(1H, s), 8.18(1H, d), 8.12(1H, d), 8.00(1H, d), |
| | 7.89(1H, d), 7.77(1H, s), 7.79(4H, d), 7.32-7.66(19H, |
| | m) |
| 1-12 | δ = 8.56(1H, d), 8.24(1H, d), 8.18(1H, d), 8.12(1H, d), |
| | 8.00(1H, d), 7.89(1H, d), 7.77(1H, s), 7.22-7.70(24H, |
| | m) |
| 1-14 | δ = 8.28(4H, d), 8.24(1H, d), 8.18(1H, d), 8.12(1H, d), |
| | 8.11(1H, d), 8.08(1H, s), 7.89(1H, d), 7.82(1H, s), |
| | 7.77(1H, s), 7.32-7.70(22H, m) |
| 1-23 | δ = 8.45(1H, d), 8.28(2H, d), 8.18(1H, d), 8.12(1H, d), |
| | 8.00(1H, d), 7.89(1H, d), 7.77(1H, s), 7.29-7.66(22H, |
| | m) |
| 1-29 | δ = 8.28(2H, d), 8.18(1H, d), 8.12(1H, d), 8.00(1H, d), |
| | 7.89(2H, d), 7.77(1H, s), 7.75(1H, d), 7.29-7.66 (21H, |
| | m) |
| 1-34 | δ = 8.24(2H, d), 8.18(1H, d), 8.12(1H, d), 8.00(1H, d), |
| | 7.89(1H, d), 7.77(1H, s), 7.29-7.70(29H, m) |
| 1-61 | δ = 8.28(4H, d), 7.89(2H, d), 7.81(1H, d), 7.32- |
| | 7.72(16H, m) |
| 1-76 | δ = 8.28(4H, d), 7.89(2H, d), 7.75(1H, d), 7.32- |
| | 7.66(16H, m) |
| 1-81 | δ = 8.45(1H, d), 8.28(4H, d), 8.00(2H, d), 7.98(1H, d), |
| | 7.89(1H, d), 7.86(1H, d), 7.32-7.66(13H, m) |
| 1-101 | δ = 8.28(4H, d), 7.93(1H, d), 7.89(1H, d), 7.87(1H, d), |
| | 7.77(1H, s), 7.28-7.66(15H, m), 1.72(6H, s) |
| 1-115 | δ = 8.28(4H, d), 7.93(1H, d), 7.89(1H, d), 7.87(1H, d), |
| | 7.77(1H, s), 7.28-7.66(21H, m), 7.11(4H, d) |
| 1-117 | δ = 8.28(4H, d), 7.89(1H, d), 7.87(1H, d), 7.28- |
| | 7.66(17H, m). 1.72(6H, s) |
| 1-188 | δ = 8.28(4H, d), 7.89(1H, d), 7.66(1H, d), 7.38- |
| | 7.54(23H, m) 7.00(1H, s), 6.69(4H, d) |
| 1-135 | δ = 8.28(4H, d), 7.89(1H, d), 7.85(2H, d), 7.66(1H, d), |
| | 7.38-7.54(23H, m), 7.32(1H, t), 7.25(2H, d), 7.00(1H, |
| | s), 6.69(4H, d) |
| 1-172 | δ = 8.28(4H, d), 7.89(1H, d), 7.66(1H, d), 7.32- |
| | 7.60(26H, m), 6.69(6H, d) |
| 1-173 | δ = 8.28(4H, d), 7.89(1H, d), 7.66(1H, d), 7.32- |
| | 7.60(25H, m), 6.89(1H, s), 7.88(1H, d), 6.69(4H, d), |
| | 6.59(1H, d) |
| 1-178 | δ = 8.28(4H, d), 7.89(1H, d), 7.66(1H, d), 7.32- |
| | 7.66(24H, m), 6.69(4H, d) |
| 1-188 | δ = 8.28(4H, d), 7.89(1H, d), 7.66(1H, d), 7.38- |
| | 7.51(10H, m), 7.20(4H, d), 6.81(2H, t), 6.63(4H, d) |
| 1-192 | δ = 8.28(4H, d), 7.89(1H, d), 7.66(1H, d), 7.32- |
| | 7.60(25H, m), 6.89(1H, s), 7.88(1H, d), 6.69(4H, d), |
| | 6.59(1H, d) |
| 1-197 | δ = 7.92(1H, s), 7.89(1H, d), 7.85(4H, d), 7.66(1H, d), |
| | 7.60(1H, s), 7.25-7.52(16H, m) |
| 1-200 | δ = 7.92(1H, s), 7.89(1H, d), 7.85(6H, d), 7.66(1H, d), |
| | 7.60(1H, s), 7.25-7.52(18H, m) |
| 1-202 | δ = 7.89(1H, d), 7.85(4H, d), 7.84(2H, d), 7.82(2H, d), |
| | 7.66 (1H, d), 7.25-7.52(18H, m) |
| 1-205 | δ = 7.89(1H, d), 7.85(4H, d), 7.66 (1H, d), 7.63(1H, s), |
| | 7.60(1H, s), 7.25-7.52(16H, m) |
| 1-208 | δ = 7.97(1H, d), 7.89(1H, d), 7.85 (4H, d), 7.79(1H, d), |
| | 7.25-7.70(21H, m) |
| 1-212 | δ = 8.24(2H, d), 7.89(1H, d), 7.32-7.70(21H, m) |
| 2-1 | δ = 8.45(1H, d), 8.28(4H, d), 8.18(1H, d), 8.12(1H, d), |
| | 8.04(1H, s), 8.00(1H, d), 7.98(1H, d), 7.77(2H, s), |
| | 7.41-7.63(15H, m), 7.29(1H, t) |
| 2-2 | δ = 8.45(1H, d), 8.23(1H, s), 8.18(1H, d), 8.12(1H, d), |
| | 8.04(1H, s), 8.00(1H, d), 7.98(1H, d), 7.79(4H, d), |
| | 7.77(2H, s), 7.41-7.63(15H, m), 7.29(1H, t) |
| 2-9 | δ = 8.45(1H, d), 8.28(4H, d), 8.24(1H, d), 8.18(1H, d), |
| | 8.12(1H, d), 8.04(1H, s), 8.00(1H, d), 7.98(1H, d), |
| | 7.77(2H, s), 7.70(1H, s), 7.41-7.63(17H, m), 7.29(1H, |
| | t) |
| 2-26 | δ = 8.45(1H, d), 8.28(4H, d), 8.04(1H, s), 7.98(1H, d), |
| | 7.89(1H, d), 7.81(1H, d), 7.77(1H, s), 7.72(1H, d), |
| | 7.71(1H, s), 7.66 (1H, d), 7.32-7.52(10H, m) |
| 2-40 | δ = 8.45(2H, d), 8.23(1H, s), 8.04(1H, s), 7.98(2H, d), |
| | 7.94(1H, d), 7.82(1H, d), 7.79(4H, d), 7.77(1H, s), |
| | 7.41-7.79(11H, m) |
| 2-41 | δ = 8.45(1H, d), 8.28(4H, d), 8.04(1H, s), 7.98(1H, d), |
| | 7.93(1H, d), 7.87(1H, d), 7.77(2H, s), 7.63(1H, d), |
| | 7.38-7.55(10H, m), 1.28(1H, t) 1.72(6H, s) |
| 2-50 | δ = 8.45(1H, d), 8.28(4H, d), 8.04(1H, s), 7.98(1H, d), |
| | 7.87(1H, d), 7.77(1H, s), 7.75(2H, d), 7.63(1H, d), |
| | 7.16-7.55(19H, m) |
| 2-51 | δ = 8.45(1H, d), 8.28(4H, d), 7.98(1H, d), 7.41- |
| | 7.54(23H, m) 7.17(1H, s), 6.69(4H, d) |
| 2-56 | δ = 8.45(1H, d), 8.28(4H, d), 8.04(1H, s), 7.98(1H, d), |
| | 7.77(1H, s), 7.41-7.54(24H, m), 6.69(6H, d) |
| 2-57 | δ = 8.45(1H, d), 8.28(4H, d), 7.98(1H, d), 7.41- |
| | 7.54(24H, m), 7.17(1H, s), 7.02(1H, s), 7.69(1H, d), |
| | 6.69(4H, d) |
| 2-60 | δ = 8.45(1H, d), 8.28(4H, d), 8.04(1H, s), 7.98(1H, d), |
| | 7.77(1H, s), 7.41-7.54(24H, m), 6.69(6H, d) |
| 2-61 | δ = 8.45(1H, d), 8.36(1H, s), 7.98(1H, d), 7.85(4H, d), |
| | 7.80(1H, s), 7.41-7.52(12H, m), 7.25(4H, d) |
| 2-63 | δ = 8.45(1H, d), 8.36(1H, s), 7.98(1H, d), 7.85(4H, d), |
| | 7.84(2H, d), 7.82(2H, d), 7.77 (1H, s), 7.41-7.52(12H, |
| | m), 7.25(4H, d) |
| 2-64 | δ = 8.45(1H, d), 8.07(1H, s), 7.98(1H, d), 7.85(4H, d), |
| | 7.80(1H, s), 7.41-7.52(12H, m), 7.25(4H, d) |
| 3-1 | δ = 8.55(1H, d), 8.04(1H, d), 7.98(1H, d), 8.00(1H, d), |
| | 7.94(1H, d), 7.77(1H, s), 7.25-7.63(23H, m) |
| 3-2 | δ = 8.55(1H, d), 8.28(2H, d), 8.18(1H, d), 8.12(1H, d), |
| | 8.00(1H, d), 7.94(1H, d), 7.79(4H, d), 7.77(1H, s), |
| | 7.25-7.63(26H, m) |
| 3-7 | δ = 8.55(1H, d), 8.28(4H, d), 8.24(1H, d), 8.18(1H, d), |
| | 8.12(1H, d), 8.00(1H, d), 7.94(1H, d), 7.77(1H, s), |
| | 7.70(1H, s), 7.25-7.63(25H, m) |
| 3-9 | δ = 8.55(1H, d), 8.45(1H, d), 8.28(2H, d), 8.18(1H, d), |
| | 8.12(1H, d), 8.11(1H, d), 8.08(1H, s), 8.00(1H, d), |
| | 7.98(1H, d), 7.94(1H, d), 7.82(1H, d), 7.77(1H, s), |
| | 7.25-7.63(22H, m) |
| 3-11 | δ = 8.55(1H, d), 8.24(2H, d), 8.18(1H, d), 8.12(1H, d), |
| | 8.00(1H, d), 7.94(1H, d), 7.77(1H, s), 7.70(2H, s), |
| | 7.25-7.58(31H, m) |
| 3-21 | δ = 8.55(1H, d), 8.28(4H, d), 7.94(1H, d), 7.89(1H, d), |
| | 7.81(1H, d), 7.25-7.71(20H, m) |
| 3-39 | δ = 8.55(1H, d), 8.45(1H, d), 7.98(1H, d), 7.94(2H, d), |
| | 7.85(4H, d), 7.82(1H, d), 7.25-7.58(26H, m) |
| 3-41 | δ = 8.55(1H, d), 8.28(4H, d), 7.94(1H, d), 7.93(1H, d), |
| | 7.87(1H, d), 7.77(1H, s), 7.28-7.63(19H, m), 1.72(6H, |
| | s) |
| 3-49 | δ = 8.55(1H, d), 8.28(4H, d), 7.94(1H, d), 7.87(1H, d), |
| | 7.25-7.63(27H, m), 7.11(4H, d) |
| 3-51 | δ = 8.55(1H, d), 8.28(4H, d), 7.94(1H, d), 7.41- |
| | 7.58(27H, m), 6.98(1H, s), 6.69(4H, d), 6.41(1H, s) |
| 3-56 | δ = 8.55(1H, d), 8.28(4H, d), 7.94(1H, d), 7.41- |
| | 7.58(31H, m), 6.69(6H, d) |
| 3-57 | δ = 8.55(1H, d), 8.28(4H, d), 7.94(1H, d), 7.41- |
| | 7.58(27H, m), 6.69(4H, d), 6.63(1H, s), 6.42(1H, s) |
| 3-60 | δ = 8.55(1H, d), 8.28(4H, d), 7.94(1H, d), 7.41- |
| | 7.58(30H, m), 6.89(1H, s), 6.88(1H, d), 6.69(4H, d), |
| | 6.59(1H, d) |
| 3-61 | δ = 8.55(1H, d), 7.94(1H, d), 7.90(1H, s), 7.85(4H, d), |
| | 7.25-7.58 (22H, m) |
| 3-63 | δ = 8.55(1H, d), 7.97(1H, d), 7.94(1H, d), 7.85(4H, d), |
| | 7.79(1H, t), 7.70(1H, d), 7.25-7.59 (24H, m) |
| 3-64 | δ = 8.55(1H, d), 7.94(1H, d), 7.85(4H, d), 7.25- |
| | 7.61(23H, m) |
| 4-3 | δ = 8.55(1H, d), 8.30(1H, d), 8.21-8.13(3H, m), 7.99- |
| | 7.89(3H, m), 7.77-7.35(17H, m), 7.20-7.16(2H, m) |
| 4-4 | δ = 8.55(1H, d), 8.30(1H, d), 8.19-8.13(2H, m), 7.99- |
| | 7.89(8H, m), 7.77-7.75(3H, m), 7.62-7.35(11H, m), |
| | 7.20-7.16(2H, m) |
| 4-7 | δ = 8.55(1H, d), 8.31-8.30(3H, d), 8.19-8.13(2H, m), |
| | 7.99-7.89(5H, m), 7.77-7.75(5H, m), 7.62-7.35(14H, m), |
| | 7.20-7.16(2H, m) |
| 4-31 | δ = 8.55(1H, d), 8.30(1H, d), 8.21-8.13(4H, m), 7.99- |
| | 7.89(4H, m), 7.77-7.35(20H, m), 7.20-7.16(2H, m) |
| 4-32 | δ = 8.55(1H, d), 8.30(1H, d), 8.21-8.13(3H, m), 7.99- |
| | 7.89(8H, m), 7.77-7.35(17H, m), 7.20-7.16(2H, m) |

TABLE 21

| Compound | FD-MS | Compound | FD-MS |
|---|---|---|---|
| 1-1 | m/z = 640.23 (C₄₅H₂₈N₄O = 640.73) | 1-2 | m/z = 639.23 (C₄₆H₂₉N₃O = 639.74) |
| 1-12 | m/z = 677.25 (C₄₉H₃₁N₃O = 677.79) | 1-14 | m/z = 716.26 (C₅₁H₃₂N₄O = 716.83) |
| 1-23 | m/z = 746.21 (C₅₁H₃₀N₄OS = 746.88) | 1-29 | m/z = 730.24 (C₅₁H₃₀N₄O = 730.81) |
| 1-34 | m/z = 792.29 (C₅₇H₃₆N₄O = 792.92) | 1-61 | m/z = 565.18 (C₃₉H₂₃N₃O₂ = 565.62) |
| 1-76 | m/z = 565.18 (C₃₉H₂₃N₃O₂ = 565.62) | 1-81 | m/z = 581.16 (C₃₉H₂₃N₃OS = 581.68) |
| 1-101 | m/z = 591.23 (C₄₂H₂₉N₃O = 591.70) | 1-115 | m/z = 715.26 (C₅₂H₃₃N₃O = 715.84) |
| 1-117 | m/z = 591.23 (C₄₂H₂₉N₃O = 591.70) | 1-121 | m/z = 718.27 (C₅₁H₃₄N₄O = 718.84) |
| 1-135 | m/z = 794.30 (C₅₇H₃₈N₄O = 794.94) | 1-172 | m/z = 794.30 (C₅₇H₃₈N₄O = 794.94) |
| 1-173 | m/z = 794.30 (C₅₇H₃₈N₄O = 794.94) | 1-178 | m/z = 718.84 (C₅₁H₃₄N₄O = 718.27) |
| 1-188 | m/z = 565.21 (C₃₉H₂₆N₄O = 566.65) | 1-192 | m/z = 794.30 (C₅₇H₃₈N₄O = 794.94) |
| 1-197 | m/z = 576.20 (C₄₀H₂₄N₄O = 576.64) | 1-200 | m/z = 652.23 (C₄₆H₂₈N₄O = 652.74) |
| 1-202 | m/z = 652.23 (C₄₆H₂₈N₄O = 652.74) | 1-205 | m/z = 576.20 (C₄₀H₂₄N₄O = 576.64) |
| 1-208 | m/z = 576.20 (C₄₀H₂₄N₄O = 576.64) | 1-212 | m/z = 652.23 (C₄₆H₂₈N₄O = 652.74) |
| 2-1 | m/z = 656.20 (C₄₅H₂₈N₄S = 656.80) | 2-2 | m/z = 655.21 (C₄₆H₂₉N₃S = 655.81) |
| 2-9 | m/z = 732.23 (C₅₁H₃2N₄S = 732.89) | 2-26 | m/z = 581.16 (C₃₉H₂₃N₃OS = 581.68) |
| 2-40 | m/z = 596.14 (C₄₀H₂₄N₂O₂ = 596.76) | 2-41 | m/z = 607.21 (C₄₂H₂₉N₃S = 607.76) |
| 2-50 | m/z = 729.22 (C₅₁H₃₁N₃S = 729.89) | 2-51 | m/z = 734.25 (C₅₁H₃₄N₄S = 734.91) |
| 2-56 | m/z = 811.00 (C₅₇H₃₈N₄S = 811.28) | 2-57 | m/z = 734.25 (C₅₁H₃₄N₄S = 734.91) |
| 2-60 | m/z = 811.00 (C₅₇H₃₈N₄S = 811.28) | 2-61 | m/z = 592.17 (C₄₀H₂₄N₄S = 592.71) |
| 2-63 | m/z = 668.20 (C₄₆H₂₈N4S = 668.81) | 2-64 | m/z = 592.17 (C₄₀H₂₄N₄S = 592.71) |
| 3-1 | m/z = 715.27 (C₅₁H₃₃N₅ = 715.84) | 3-2 | m/z = 790.31 (C₅₈H₃₈N₄ = 790.95) |
| 3-7 | m/z = 791.30 (C₅₇H₃₇N₅ = 791.94) | 3-9 | m/z = 821.26 (C₅₇H₃₅N₅S = 821.99) |
| 3-11 | m/z = 867.34 (C₆₃H₄₁N₅ = 868.03) | 3-21 | m/z = 640.23 (C₄₅H₂₈N₄O = 640.73) |
| 3-39 | m/z = 808.27 (C₅₇H₃₆N₄S = 808.99) | 3-41 | m/z = 666.28 (C₄₈H₃₄N₄ = 666.81) |
| 3-49 | m/z = 790.31 (C₅₈H₃₈N₄ = 790.95) | 3-51 | m/z = 793.32 (C₅₇H₃₉N₅ = 793.95) |
| 3-56 | m/z = 869.35 (C₆₃H₄₃N₅ = 870.05) | 3-57 | m/z = 793.32 (C₅₇H₃₉N₅ = 793.95) |
| 3-60 | m/z = 869.35 (C₆₃H₄₃N₅ = 870.05) | 3-61 | m/z = 651.24 (C₄₆H₂₉N₅ = 651.76) |
| 3-63 | m/z = 727.27 (C₅₂H₃₃N₅ = 727.85) | 3-64 | m/z = 651.24 (C₄₆H₂₉N₅ = 651.76) |
| 4-3 | m/z = 560.23 (C₄₂H₂₈N₂ = 560.70) | 4-4 | m/z = 560.23 (C₄₂H₂₈N₂ = 560.70) |
| 4-7 | m/z = 636.26 (C₄₈H₃₂N₂ = 636.80) | 4-31 | m/z = 636.26 (C₄₈H₃₂N₂ = 636.80) |
| 4-32 | m/z = 636.26 (C₄₈H₃₂N₂ = 636.80) | | |

### <Experimental Example 1>—Manufacture of Organic Light Emitting Device

A glass substrate on which indium tin oxide (ITO) was coated as a thin film to a thickness of 1500 Å was cleaned with distilled water ultrasonic waves. After the cleaning with distilled water was finished, the substrate was ultrasonic cleaned with solvents such as acetone, methanol and isopropyl alcohol, then dried, and ultraviolet ozone (UVO) treatment was conducted for 5 minutes using UV in an ultraviolet (UV) cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 400 Å using the compound described in Chemical Formula 1 as a host, and Ir(ppy)₃ was deposited as a green phosphorescent dopant by 7% doping with respect to the deposited thickness of the light emitting layer. After that, bathocuproine (BCP) was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁶ torr to 10⁻⁸ torr for each material to be used in the OLED manufacture.

For the organic electroluminescent devices manufactured as above, electroluminescent (EL) properties were measured using M7000 manufactured by McScience Inc., and with the measurement results, T90 was measured when standard luminance was 6,000 cd/m² using a lifetime measurement system (M6000) manufactured by McScience Inc.

Results of measuring driving voltage, light emission efficiency, color coordinate (CIE) and lifetime of the organic light emitting devices manufactured according to the present disclosure are as shown in the following Table 22.

TABLE 22

| | Light | | | | |
|---|---|---|---|---|---|
| | Emitting | Driving | | Color | Life- |
| | Layer | Voltage | Efficiency | Coordinate | time |
| | Compound | (V) | (cd/A) | (x, y) | (T₉₀) |
| Example 1 | 1-1 | 4.11 | 75.2 | (0.247, | 227 |
| | | | | 0.727) | |
| Example 2 | 1-2 | 4.35 | 75.7 | (0.251, | 221 |
| | | | | 0.714) | |
| Example 3 | 1-12 | 4.42 | 72.2 | (0.231, | 211 |
| | | | | 0.711) | |
| Example 4 | 1-14 | 4.15 | 74.2 | (0.241, | 221 |
| | | | | 0.714) | |
| Example 5 | 1-23 | 4.33 | 76.9 | (0.242, | 239 |
| | | | | 0.713) | |
| Example 6 | 1-29 | 4.40 | 76.8 | (0.231, | 249 |
| | | | | 0.711) | |
| Example 7 | 1-34 | 4.41 | 76.8 | (0.241, | 285 |
| | | | | 0.711) | |
| Example 8 | 1-61 | 4.31 | 79.5 | (0.231, | 331 |
| | | | | 0.711) | |
| Example 9 | 1-76 | 4.13 | 79.2 | (0.247, | 355 |
| | | | | 0.727) | |
| Example 10 | 1-81 | 4.33 | 77.2 | (0.246, | 297 |
| | | | | 0.717) | |
| Example 11 | 1-101 | 4.37 | 77.2 | (0.247, | 293 |
| | | | | 0.726) | |
| Example 12 | 1-115 | 4.46 | 76.1 | (0.251, | 290 |
| | | | | 0.717) | |
| Example 13 | 1-117 | 4.39 | 76.9 | (0.231, | 293 |
| | | | | 0.714) | |
| Example 14 | 1-121 | 4.32 | 71.5 | (0.251, | 210 |
| | | | | 0.713) | |
| Example 15 | 1-135 | 4.66 | 71.1 | (0.248, | 212 |
| | | | | 0.711) | |
| Example 16 | 1-172 | 4.45 | 72.8 | (0.251, | 238 |
| | | | | 0.714) | |
| Example 17 | 1-173 | 4.69 | 75.2 | (0.243, | 224 |
| | | | | 0.714) | |
| Example 18 | 1-178 | 4.66 | 71.2 | (0.251, | 231 |
| | | | | 0.714) | |
| Example 19 | 1-188 | 4.64 | 70.5 | (0.247, | 210 |
| | | | | 0.727) | |
| Example 20 | 1-192 | 4.50 | 71.9 | (0.251, | 201 |
| | | | | 0.714) | |
| Example 21 | 1-197 | 4.45 | 72.8 | (0.251, | 176 |
| | | | | 0.713) | |
| Example 22 | 1-200 | 4.66 | 71.1 | (0.241, | 175 |
| | | | | 0.715) | |
| Example 23 | 1-202 | 4.41 | 68.4 | (0.246, | 176 |
| | | | | 0.717) | |
| Example 24 | 1-205 | 4.38 | 76.4 | (0.241, | 171 |
| | | | | 0.711) | |
| Example 25 | 1-208 | 4.69 | 69.2 | (0.231, | 166 |
| | | | | 0.712) | |
| Example 26 | 1-212 | 4.66 | 71.1 | (0.248, | 164 |
| | | | | 0.711) | |
| Example 26 | 2-1 | 5.23 | 59.0 | (0.247, | 211 |
| | | | | 0.727) | |
| Example 25 | 2-2 | 5.43 | 57.1 | (0.241, | 200 |
| | | | | 0.715) | |
| Example 26 | 2-9 | 5.55 | 55.4 | (0.246, | 196 |
| | | | | 0.717) | |
| Example 27 | 2-26 | 4.63 | 71.1 | (0.241, | 283 |
| | | | | 0.711) | |
| Example 28 | 2-40 | 5.55 | 69.2 | (0.243, | 270 |
| | | | | 0.714) | |
| Example 29 | 2-41 | 5.60 | 61.2 | (0.233, | 254 |
| | | | | 0.715) | |
| Example 30 | 2-50 | 5.62 | 59.5 | (0.246, | 251 |
| | | | | 0.717) | |
| Example 31 | 2-51 | 5.23 | 57.2 | (0.241, | 167 |
| | | | | 0.710) | |
| Example 32 | 2-56 | 5.41 | 56.2 | (0.247, | 166 |
| | | | | 0.727) | |
| Example 33 | 2-57 | 5.59 | 57.9 | (0.248, | 168 |
| | | | | 0.711) | |
| Example 34 | 2-60 | 5.62 | 55.2 | (0.243, | 165 |
| | | | | 0.714) | |
| Example 35 | 2-61 | 4.71 | 53.4 | (0.233, | 152 |
| | | | | 0.701) | |
| Example 36 | 2-63 | 4.75 | 53.6 | (0.243, | 150 |
| | | | | 0.716) | |
| Example 37 | 2-64 | 4.79 | 54.1 | (0.241, | 148 |
| | | | | 0.711) | |
| Example 38 | 3-1 | 5.50 | 53.4 | (0.242, | 177 |
| | | | | 0.713) | |
| Example 39 | 3-2 | 4.49 | 54.1 | (0.241, | 172 |
| | | | | 0.710) | |
| Example 40 | 3-7 | 5.57 | 53.7 | (0.231, | 175 |
| | | | | 0.711) | |
| Example 41 | 3-9 | 4.45 | 56.9 | (0.233, | 188 |
| | | | | 0.701) | |
| Example 42 | 3-11 | 4.33 | 57.2 | (0.247, | 191 |
| | | | | 0.727) | |
| Example 43 | 3-21 | 5.69 | 63.1 | (0.246, | 246 |
| | | | | 0.717) | |
| Example 44 | 3-39 | 5.62 | 61.2 | (0.243, | 235 |
| | | | | 0.716) | |
| Example 45 | 3-41 | 5.21 | 58.2 | (0.241, | 215 |
| | | | | 0.711) | |
| Example 46 | 3-49 | 5.35 | 59.4 | (0.231, | 217 |
| | | | | 0.713) | |
| Example 47 | 3-51 | 4.75 | 51.2 | (0.254, | 139 |
| | | | | 0.724) | |
| Example 44 | 3-56 | 4.81 | 55.9 | (0.243, | 134 |
| | | | | 0.693) | |
| Example 49 | 3-57 | 4.73 | 54.3 | (0.234, | 132 |
| | | | | 0.714) | |
| Example 50 | 3-60 | 4.73 | 52.2 | (0.251, | 131 |
| | | | | 0.714) | |
| Example 51 | 3-61 | 5.33 | 54.9 | (0.233, | 118 |
| | | | | 0.701) | |
| Example 52 | 3-63 | 5.62 | 56.4 | (0.243, | 115 |
| | | | | 0.716) | |
| Example 53 | 3-64 | 5.74 | 55.2 | (0.251, | 113 |
| | | | | 0.724) | |
| Comparative | Ref. 1 | 5.14 | 48.9 | (0.246, | 47 |
| Example 1 | | | | 0.717) | |
| Comparative | Ref. 2 | 5.62 | 46.0 | (0.243, | 45 |
| Example 2 | | | | 0.690) | |
| Comparative | Ref. 3 | 5.54 | 45.9 | (0.246, | 33 |
| Example 3 | | | | 0.686) | |
| Comparative | Ref. 4 | 5.64 | 43.9 | (0.236, | 41 |
| Example 4 | | | | 0.696) | |
| Comparative | Ref. 5 | 5.26 | 47.6 | (0.255, | 35 |
| Example 5 | | | | 0.698) | |
| Comparative | Ref. 6 | 5.10 | 44.7 | (0.244, | 30 |
| Example 6 | | | | 0.684) | |
| Comparative | Ref. 7 | 5.33 | 49.2 | (0.247, | 93 |
| Example 7 | | | | 0.727) | |
| Comparative | Ref. 8 | 5.66 | 45.8 | (0.233, | 68 |
| Example 8 | | | | 0.701) | |
| Comparative | Ref. 9 | 5.31 | 45.7 | (0.243, | 77 |
| Example 9 | | | | 0.693) | |
| Comparative | Ref. 10 | 5.43 | 46.3 | (0.253, | 32 |
| Example 10 | | | | 0.691) | |
| Comparative | Ref. 11 | 5.37 | 48.6 | (0.247, | 85 |
| Example 11 | | | | 0.725) | |
| Comparative | Ref. 12 | 5.35 | 47.3 | (0.255, | 81 |
| Example 12 | | | | 0.724) | |
| Comparative | Ref. 13 | 5.50 | 44.7 | (0.236, | 62 |
| Example 13 | | | | 0.717) | |
| Comparative | Ref. 14 | 5.11 | 44.2 | (0.255, | 74 |
| Example 14 | | | | 0.690) | |
| Comparative | 4-3 | 4.83 | 50.9 | (0.233, | 91 |
| Example 15 | | | | 0.703) | |
| Comparative | 4-4 | 4.69 | 69.2 | (0.231, | 96 |
| Example 16 | | | | 0.712) | |
| Comparative | 4-7 | 5.21 | 57.0 | (0.247, | 85 |
| Example 17 | | | | 0.727) | |
| Comparative | 4-31 | 4.75 | 51.2 | (0.254, | 79 |
| Example 18 | | | | 0.724) | |
| Comparative | 4-32 | 4.48 | 70.2 | (0.241, | 86 |
| Example 19 | | | | 0.714) | |

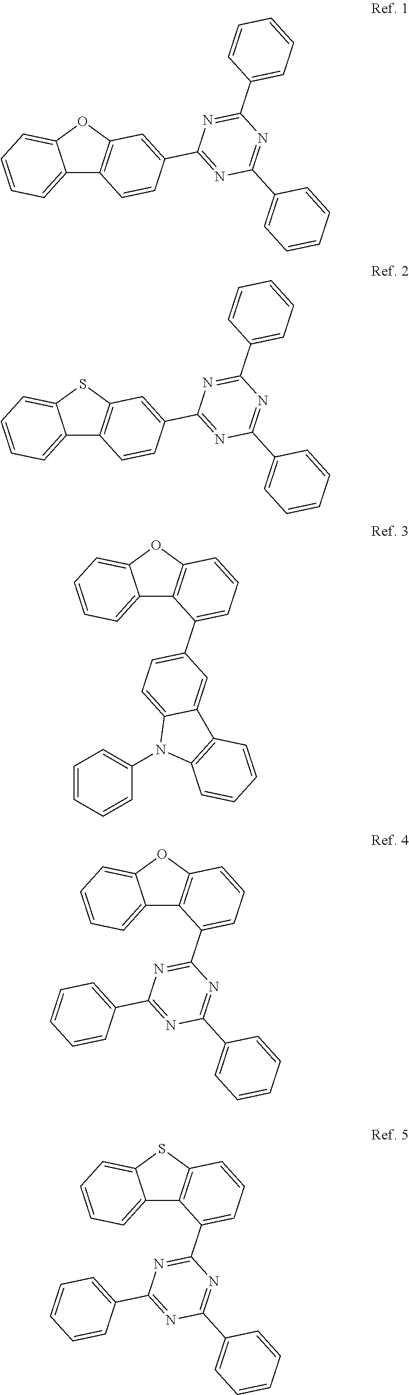

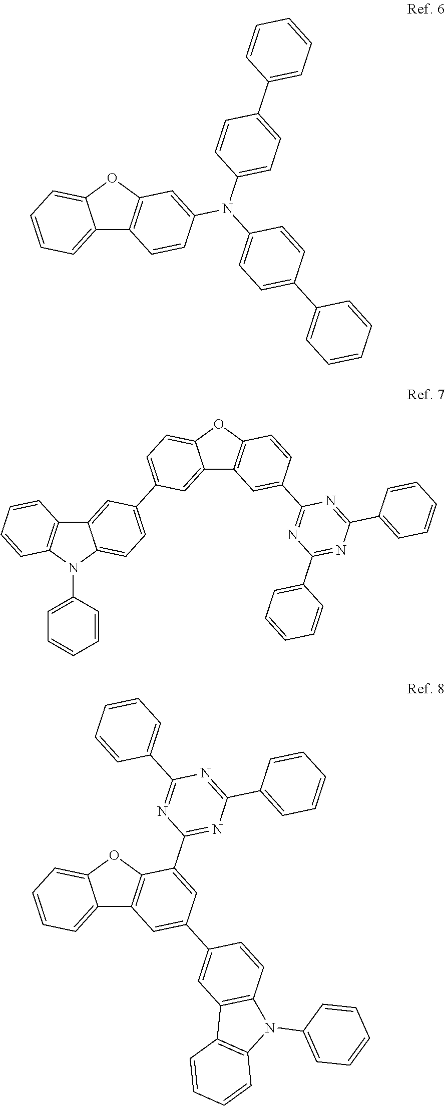 [SMILES: c1ccc(-c2ccc(N(c3ccc(-c4ccccc4)cc3)c3ccc4c(c3)oc3ccccc34)cc2)cc1 ; c1ccc(-c2nc(-c3ccccc3)nc(-c3ccc4oc5ccc(-c6ccc7c(c6)c6ccccc6n7-c6ccccc6)cc5c4c3)n2)cc1 ; c1ccc(-c2nc(-c3ccccc3)nc(-c3cc(-c4ccc5c(c4)c4ccccc4n5-c4ccccc4)cc4c3oc3ccccc34)n2)cc1]

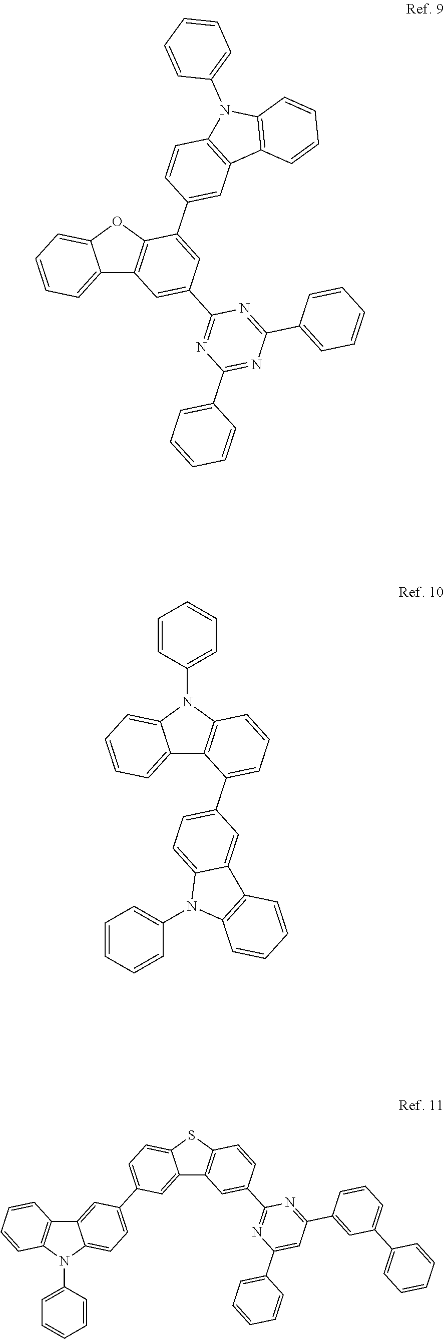 [SMILES: c1ccc(-c2nc(-c3ccccc3)nc(-c3cc(-c4ccc5c(c4)c4ccccc4n5-c4ccccc4)c4oc5ccccc5c4c3)n2)cc1 ; c1ccc(-n2c3ccccc3c3cc(-c4cccc5c4c4ccccc4n5-c4ccccc4)ccc32)cc1 ; c1ccc(-c2cccc(-c3cc(-c4ccccc4)nc(-c4ccc5sc6ccc(-c7ccc8c(c7)c7ccccc7n8-c7ccccc7)cc6c5c4)n3)c2)cc1]

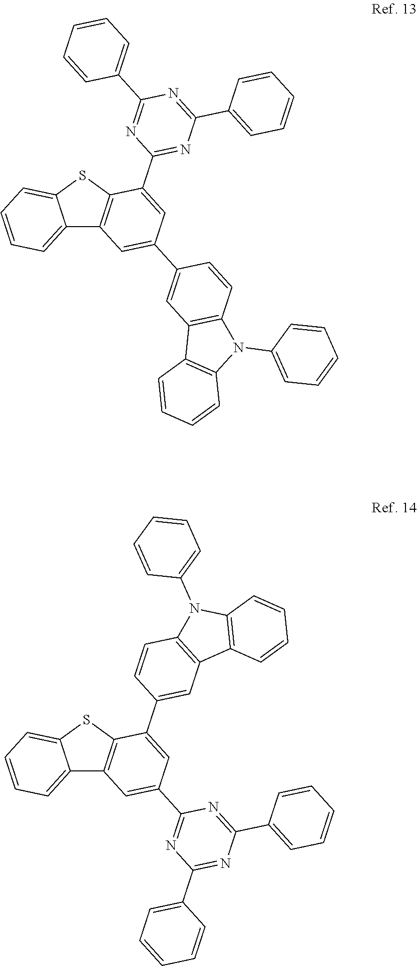 [SMILES: c1ccc(-c2nc(-c3ccccc3)nc(-c3cc(-c4ccc5c(c4)c4ccccc4n5-c4ccccc4)cc4c3sc3ccccc34)n2)cc1 ; c1ccc(-c2nc(-c3ccccc3)nc(-c3cc(-c4ccc5c(c4)c4ccccc4n5-c4ccccc4)c4sc5ccccc5c4c3)n2)cc1]

### <Experimental Example 2>—Manufacture of Organic Light Emitting Device

A glass substrate on which indium tin oxide (ITO) was coated as a thin film to a thickness of 1500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 400 Å in one source of supply after pre-mixing one type of the compound described in Chemical Formula 1 and one type of the compound described in Chemical Formula 2 as a host, and Ir(ppy)₃ was deposited as a green phosphorescent dopant by 7% doping with respect to the deposited thickness of the light emitting layer. After that, BCP was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic electroluminescent device was manufactured.

Meanwhile, all the organic compounds required to manufacture the OLED were vacuum sublimation purified under 10⁻⁶ torr to 10⁻⁸ torr for each material to be used in the OLED manufacture.

For the organic electroluminescent devices manufactured as above, electroluminescent (EL) properties were measured using M7000 manufactured by McScience Inc., and with the measurement results, T₉₀ was measured when standard luminance was 6,000 cd/m² using a lifetime measurement system (M6000) manufactured by McScience Inc.

Results of measuring driving voltage, light emission efficiency, color coordinate (CIE) and lifetime of the organic light emitting devices manufactured according to the present disclosure are as shown in the following Table 23.

TABLE 23

| | Light | | | | | |
|---|---|---|---|---|---|---|
| | Emitting | | Driving | | Color | |
| | Layer | | Voltage | Efficiency | Coordinate | Lifetime |
| | Compound | Ratio | (V) | (cd/A) | (x, y) | (T₉₀) |
| Example 50 | 1-34: | 1:8 | 4.73 | 54.2 | (0.233, | 377 |
| | 4-3 | | | | 0.714) | |
| Example 51 | | 1:5 | 4.71 | 57.2 | (0.243, | 385 |
| | | | | | 0.714) | |
| Example 52 | | 1:2 | 4.35 | 79.2 | (0.241, | 498 |
| | | | | | 0.714) | |
| Example 53 | | 1:1 | 4.41 | 75.8 | (0.231, | 492 |
| | | | | | 0.711) | |
| Example 54 | | 2:1 | 4.67 | 71.2 | (0.251, | 443 |
| | | | | | 0.714) | |
| Example 55 | | 5:1 | 4.32 | 68.3 | (0.241, | 375 |
| | | | | | 0.711) | |
| Example 56 | | 8:1 | 4.21 | 67.0 | (0.247, | 362 |
| | | | | | 0.727) | |
| Example 57 | 1-152: | 1:2 | 4.36 | 75.2 | (0.247, | 439 |
| | 4-4 | | | | 0.729) | |
| Example 58 | | 1:1 | 4.44 | 73.2 | (0.231, | 423 |
| | | | | | 0.711) | |
| Example 59 | | 2:1 | 4.67 | 71.5 | (0.247, | 410 |
| | | | | | 0.729) | |
| Example 60 | 1-182: | 1:2 | 4.38 | 76.3 | (0.251, | 427 |
| | 4-7 | | | | 0.714) | |
| Example 61 | | 1:1 | 4.49 | 72.5 | (0.241, | 412 |
| | | | | | 0.714) | |
| Example 62 | | 2:1 | 4.69 | 69.1 | (0.231, | 403 |
| | | | | | 0.711) | |
| Example 63 | 1-101: | 1:2 | 4.34 | 72.8 | (0.233, | 518 |
| | 4-31 | | | | 0.711) | |
| Example 64 | | 1:1 | 4.43 | 70.7 | (0.241, | 515 |
| | | | | | 0.711) | |
| Example 65 | | 2:1 | 4.58 | 69.3 | (0.243, | 508 |
| | | | | | 0.714) | |
| Example 66 | 1-81: | 1:2 | 4.33 | 73.2 | (0.247, | 521 |
| | 4-31 | | | | 0.723) | |
| Example 67 | | 1:1 | 4.45 | 70.5 | (0.241, | 481 |
| | | | | | 0.712) | |
| Example 68 | | 2:1 | 4.61 | 68.4 | (0.231, | 443 |
| | | | | | 0.716) | |
| Example 69 | | 1:2 | 4.30 | 75.2 | (0.247, | 578 |
| | | | | | 0.729) | |
| Example 70 | 1-76: | 1:1 | 4.48 | 70.9 | (0.241, | 447 |
| | 4-32 | | | | 0.718) | |
| Example 71 | | 2:1 | 4.69 | 69.2 | (0.231, | 426 |
| | | | | | 0.717) | |
| Example 72 | | 1:2 | 4.33 | 74.2 | (0.241, | 482 |
| | | | | | 0.714) | |
| Example 73 | 2-26: | 1:1 | 4.42 | 72.2 | (0.231, | 453 |
| | 4-32 | | | | 0.711) | |
| Example 74 | | 2:1 | 4.66 | 71.2 | (0.251, | 429 |
| | | | | | 0.714) | |
| Example 75 | | 1:2 | 4.38 | 76.4 | (0.241, | 452 |
| | | | | | 0.711) | |
| Example 76 | 3-21: | 1:1 | 4.45 | 72.8 | (0.251, | 443 |
| | 4-32 | | | | 0.714) | |
| Example 77 | | 2:1 | 4.66 | 71.1 | (0.241, | 428 |
| | | | | | 0.711) | |
| Comparative | | 1:2 | 4.82 | 56.4 | (0.241, | 379 |
| Example 20 | | | | | 0.714) | |
| Comparative | Ref. 7: | 1:1 | 4.71 | 54.8 | (0.233, | 370 |
| Example 21 | 4-32 | | | | 0.711) | |
| Comparative | | 2:1 | 4.75 | 54.1 | (0.231, | 366 |
| Example 22 | | | | | 0.729) | |

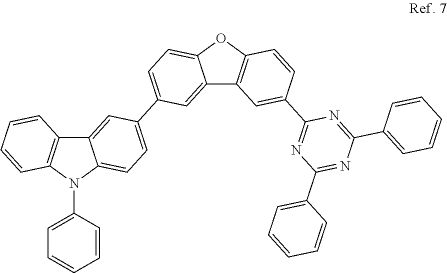 [SMILES: c1ccc(-c2nc(-c3ccccc3)nc(-c3ccc4oc5ccc(-c6ccc7c(c6)c6ccccc6n7-c6ccccc6)cc5c4c3)n2)cc1]

As can be seen from the results of Table 22, the organic electroluminescent device using the light emitting layer material of the organic electroluminescent device of the present disclosure had lower driving voltage, and significantly improved lifetime as well as having enhanced light emission efficiency compared to Comparative Examples 1 to 14.

Based on the results of Tables 22 and 23, more superior efficiency and lifetime effects were obtained when comprising both the compound of Chemical Formula 1 and the compound of Chemical Formula 2. Such results may lead to a forecast that an exciplex phenomenon occurs when comprising the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may decrease, which resultantly helps with enhancement in the lifetime. In the present disclosure, it was identified that excellent device properties were obtained when using the heterocyclic compound of Chemical Formula 2 to have a donor role and the compound of Chemical Formula 1 to have an acceptor role as a light emitting layer host.

It was identified that, when there was no substituent of any one of -(L1)m-N-Het and -(L2)p-(Z1)q of Chemical Formula 1 of the present application as in the compounds of Comparative Examples 1 to 6 and 10, a balance between holes and electrons was broken in the light emitting layer, and a lifetime was reduced.

The compounds of Comparative Examples 7 to 9 and 11 to 14 are different from the compounds of the present disclosure in the position of substitution, and in the compounds of Comparative Examples 7 to 9 and 11 to 14, the HOMO orbital is delocalized side by side from dibenzofuran to carbazole, and it was identified that this increased hole mobility compared to the HOMO orbital being delocalized vertically from dibenzofuran to carbazole as in the compounds of the present application breaking a balance between holes and electrons in the light emitting layer, and as a result, a lifetime was reduced.

1. A heterocyclic compound represented by the following Chemical Formula 1: 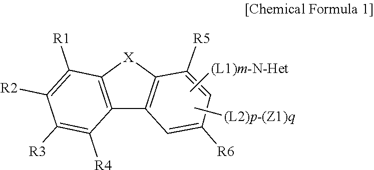
wherein, in Chemical Formula 1,
N-Het is a monocyclic or polycyclic heterocyclic group substituted or unsubstituted and comprising one or more Ns;
L1 and L2 are the same as or different from each other, and each independently a direct bond; a substituted or unsubstituted arylene group; or a substituted or unsubstituted heteroarylene group;
Z1 is selected from the group consisting of deuterium; halogen; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —P(═O)RR′; —SiRR′R″; and a substituted or unsubstituted amine group;
X is O; S; or NR7;
R7 is a substituted or unsubstituted alkyl group; a substituted or unsubstituted aryl group; or a substituted or unsubstituted heteroaryl group;
R1 to R6 are the same as or different from each other, and each independently selected from the group consisting of hydrogen; deuterium; halogen; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —P(═O)RR′; —SiRR′R″; and a substituted or unsubstituted amine group, or two or more groups adjacent to each other bond to each other to form a substituted or unsubstituted aliphatic or aromatic hydrocarbon ring or a substituted or unsubstituted heteroring;
R, R′ and R″ are the same as or different from each other, and each independently a substituted or unsubstituted alkyl group; a substituted or unsubstituted aryl group; or a substituted or unsubstituted heteroaryl group;
m and p are an integer of 0 to 3; and
q is an integer of 1 to 6.

2. The heterocyclic compound of claim 1, wherein Chemical Formula 1 is represented by the following Chemical Formula 3 or Chemical Formula 4: 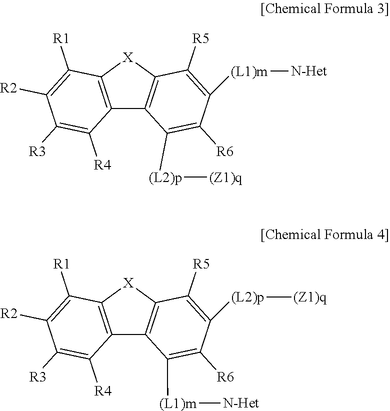
in Chemical Formulae 3 and 4,
R1 to R6, L1, L2, Z1, N-Het, X, m, p and q have the same definitions as in Chemical Formula 1.

3. The heterocyclic compound of claim 1, wherein the “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R′; —P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted; and
R, R′ and R″ have the same definitions as in Chemical Formula 1.

4. The heterocyclic compound of claim 1, wherein Z1 is —CN; or a substituted or unsubstituted amine group, or represented by the following Chemical Formula 1-1: 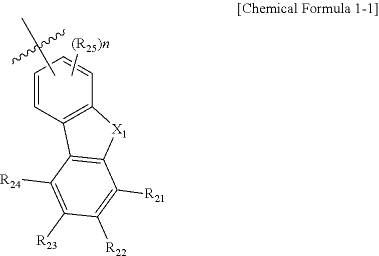
in Chemical Formula 1-1, [symbol]
means a site linked to L2 of Chemical Formula 1;
X₁ is O; S; NR₃₁; or CR₃₂R₃₃;
R₂₁ to R₂₅ are the same as or different from each other, and each independently selected from the group consisting of hydrogen; deuterium; halogen: —CN; a substituted or unsubstituted alkyl group; a substituted or unsubstituted aryl group; and a substituted or unsubstituted heteroaryl group, or two or more groups adjacent to each other bond to each other to form a substituted or unsubstituted aromatic ring;
n is an integer of 0 to 3; and
R₃₁ to R₃₃ are the same as or different from each other, and each independently selected from the group consisting of a substituted or unsubstituted alkyl group; a substituted or unsubstituted aryl group; and a substituted or unsubstituted heteroaryl group, or two or more groups adjacent to each other bond to each other to form a substituted or unsubstituted aromatic ring.

5. The heterocyclic compound of claim 1, wherein the N-Het is a monocyclic or polycyclic C2 to C60 heterocyclic group unsubstituted or substituted with one or more substituents selected from the group consisting of a C6 to C60 aryl group and a C2 to C60 heteroaryl group, and comprising one or more Ns.

6. The heterocyclic compound of claim 1, wherein R1 to R6 are hydrogen.

7. The heterocyclic compound of claim 1, wherein Chemical Formula 1 is represented by any one of the following compounds:

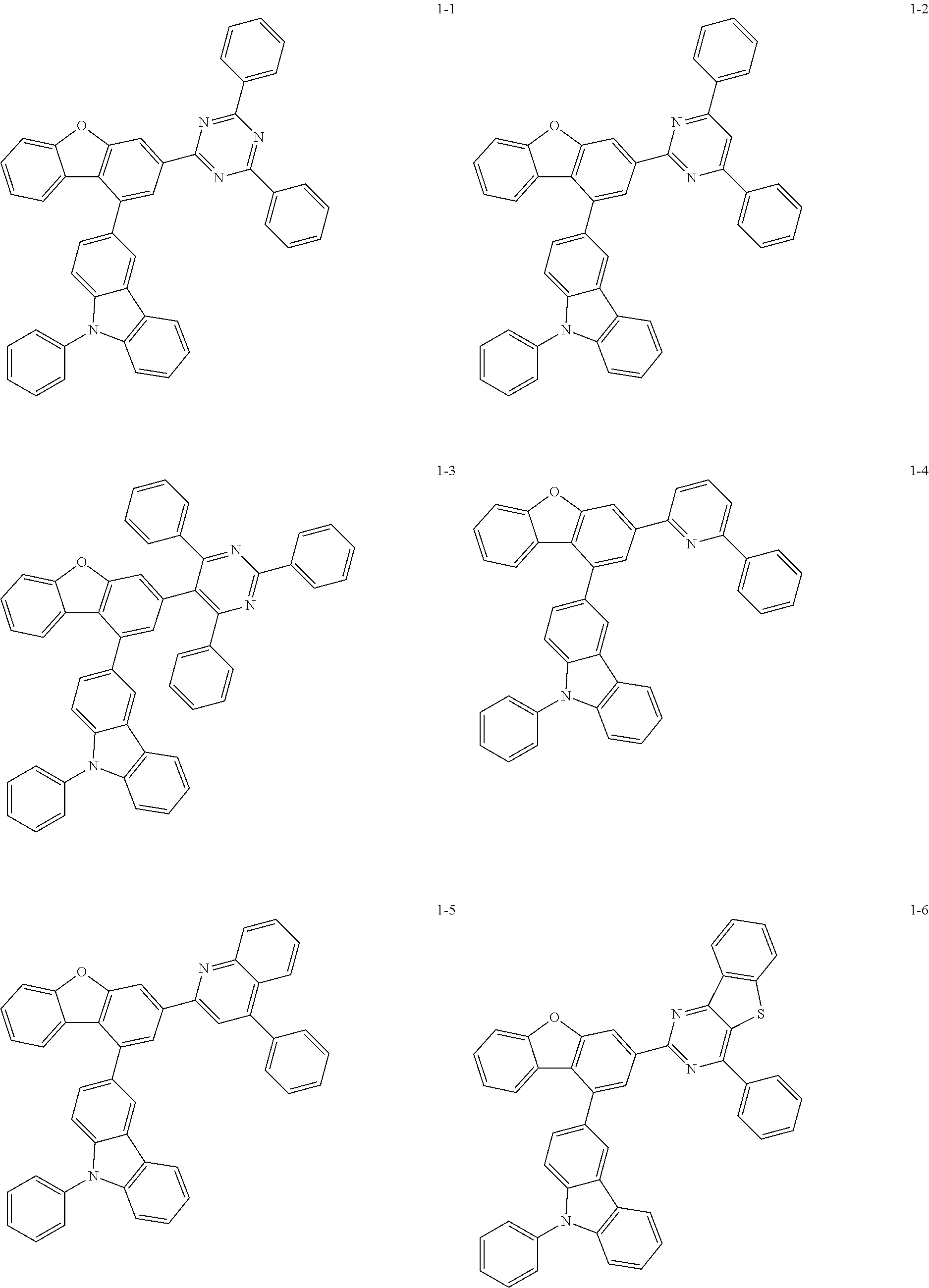

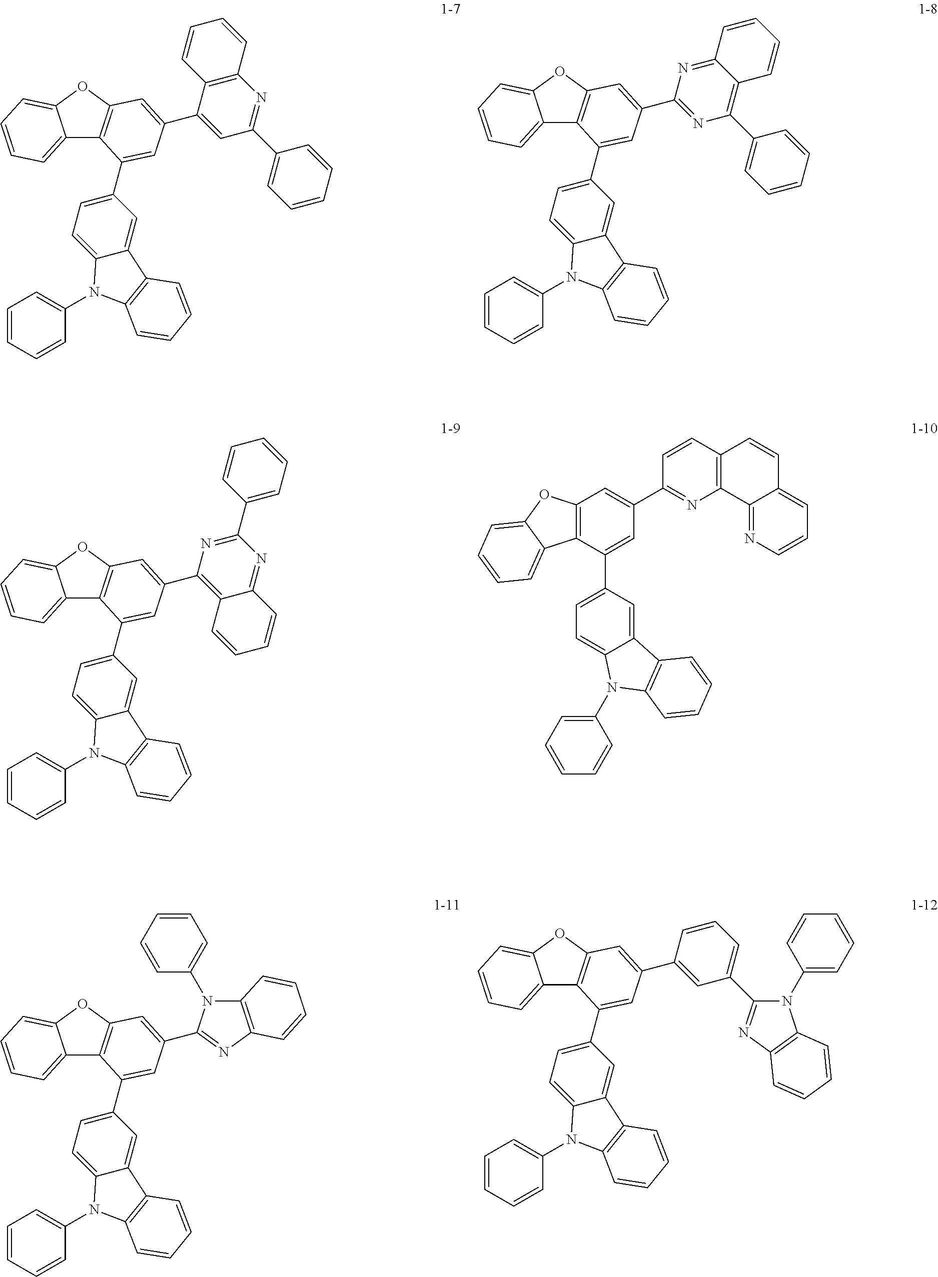

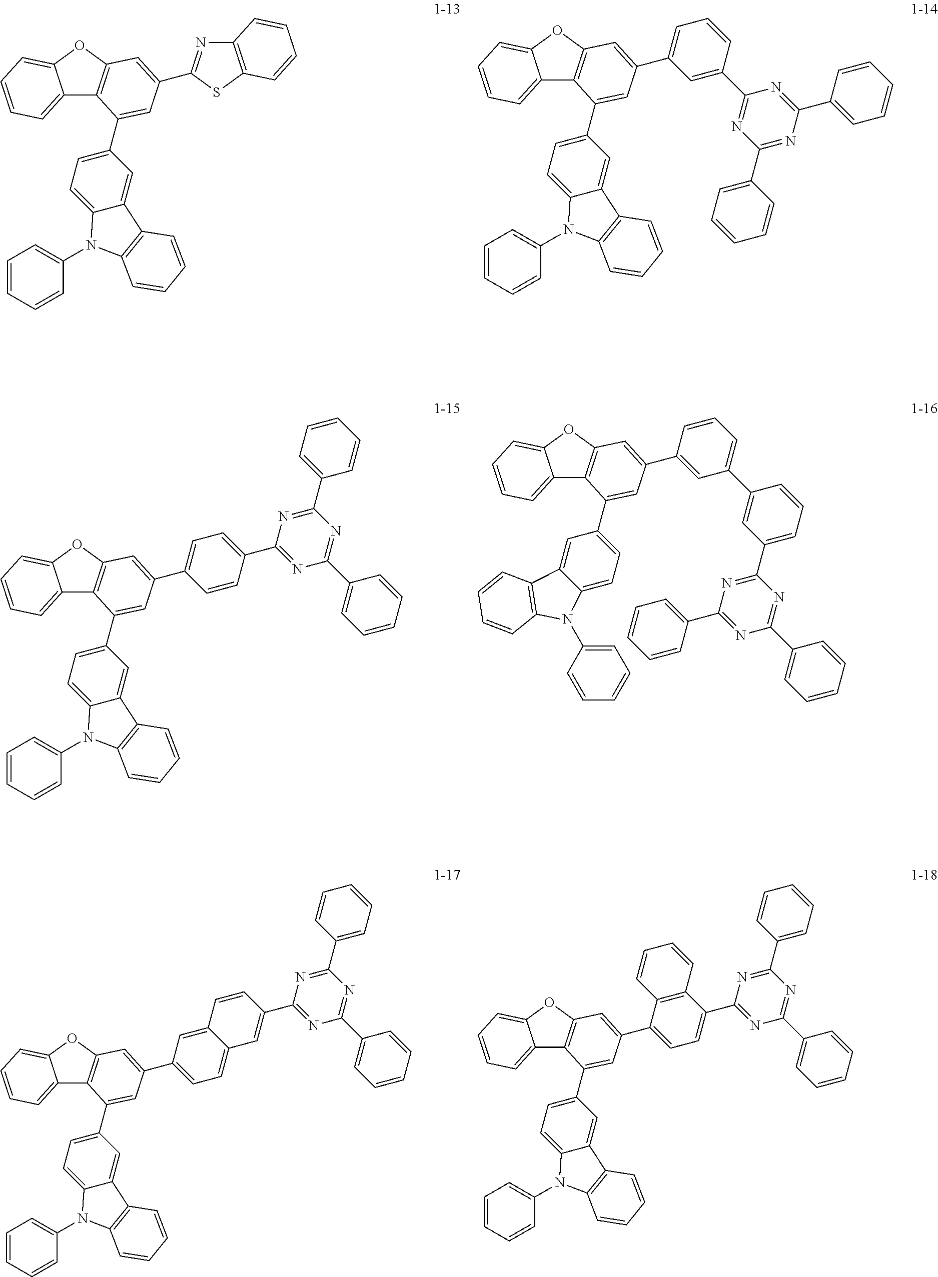

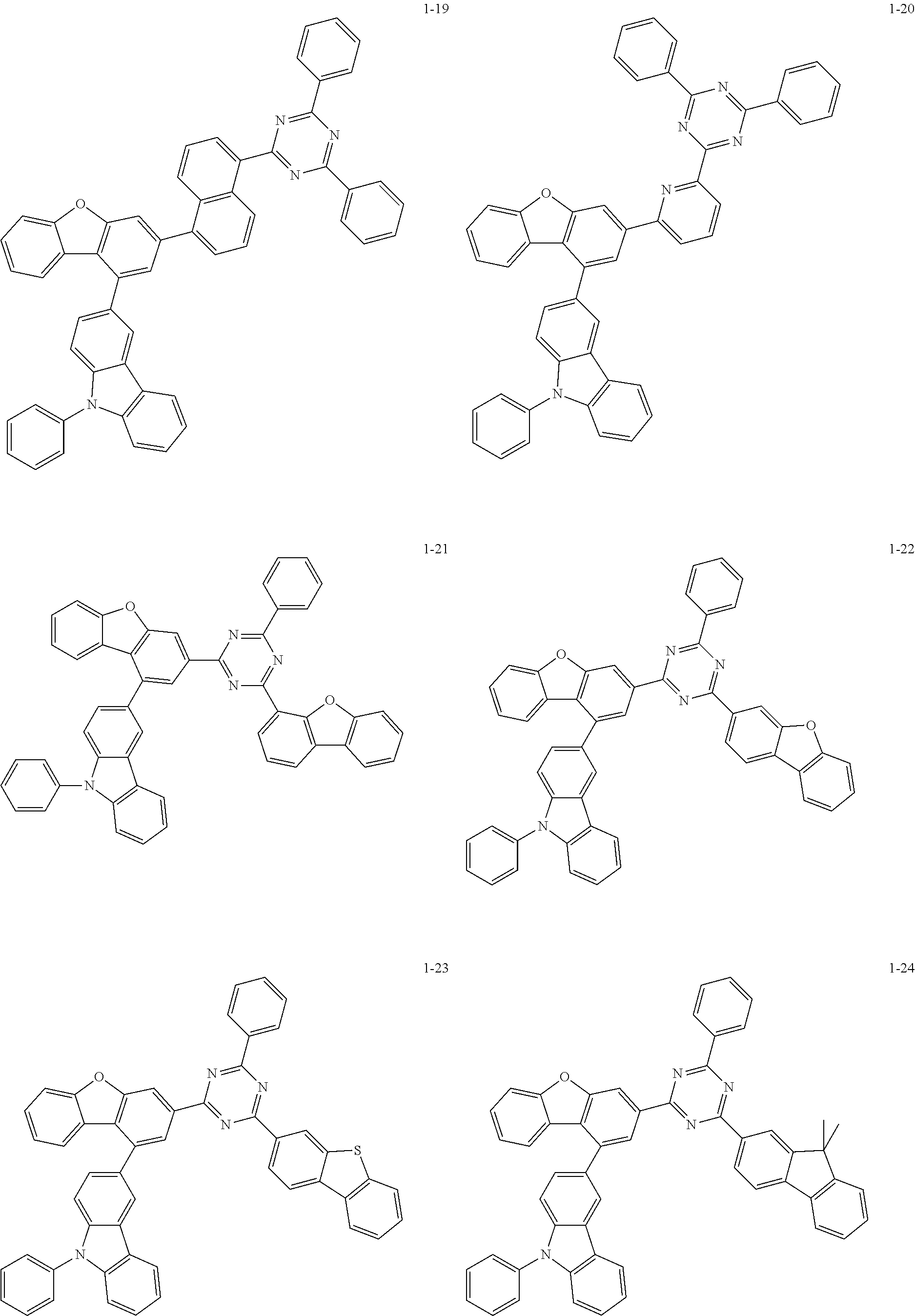

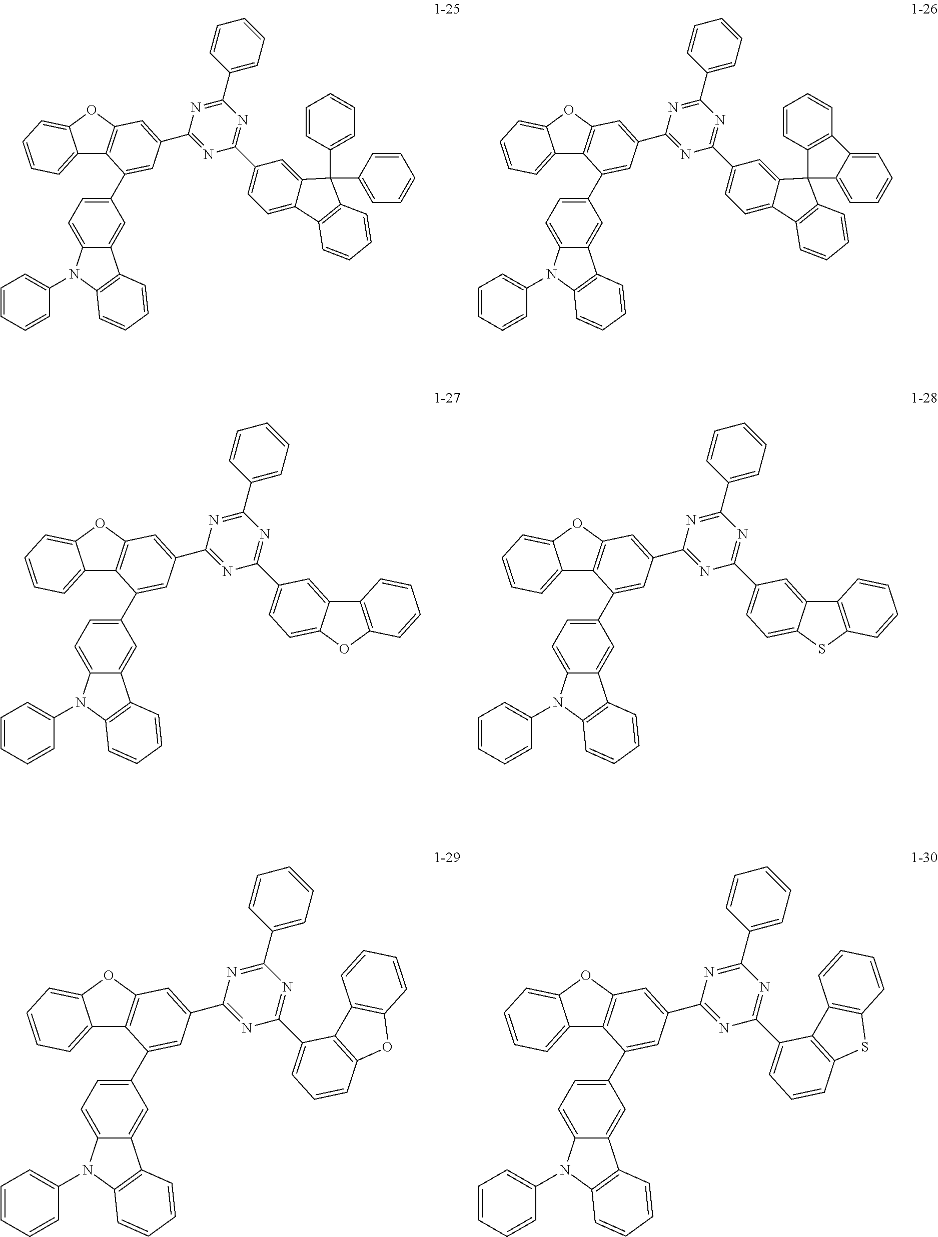

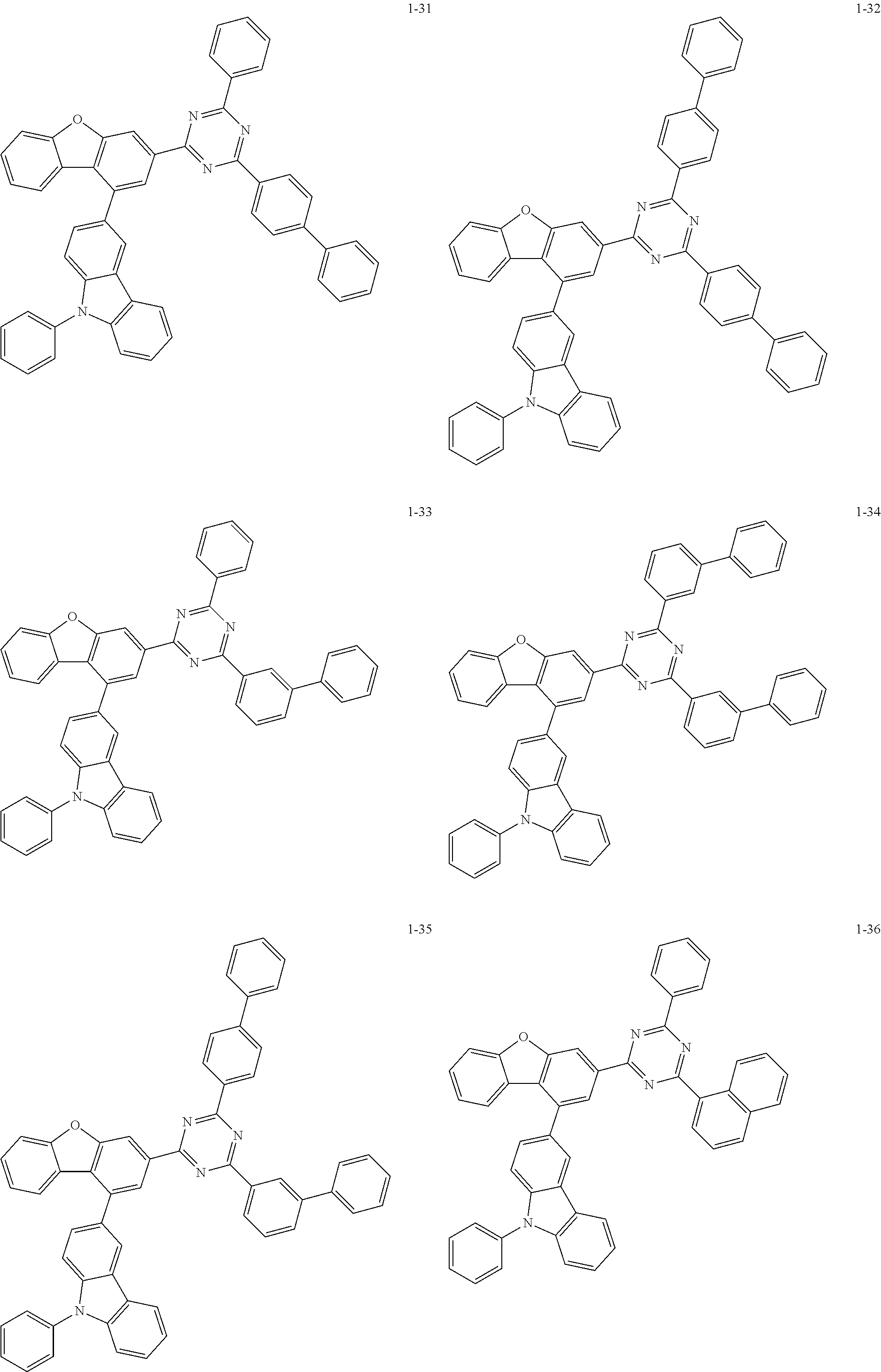

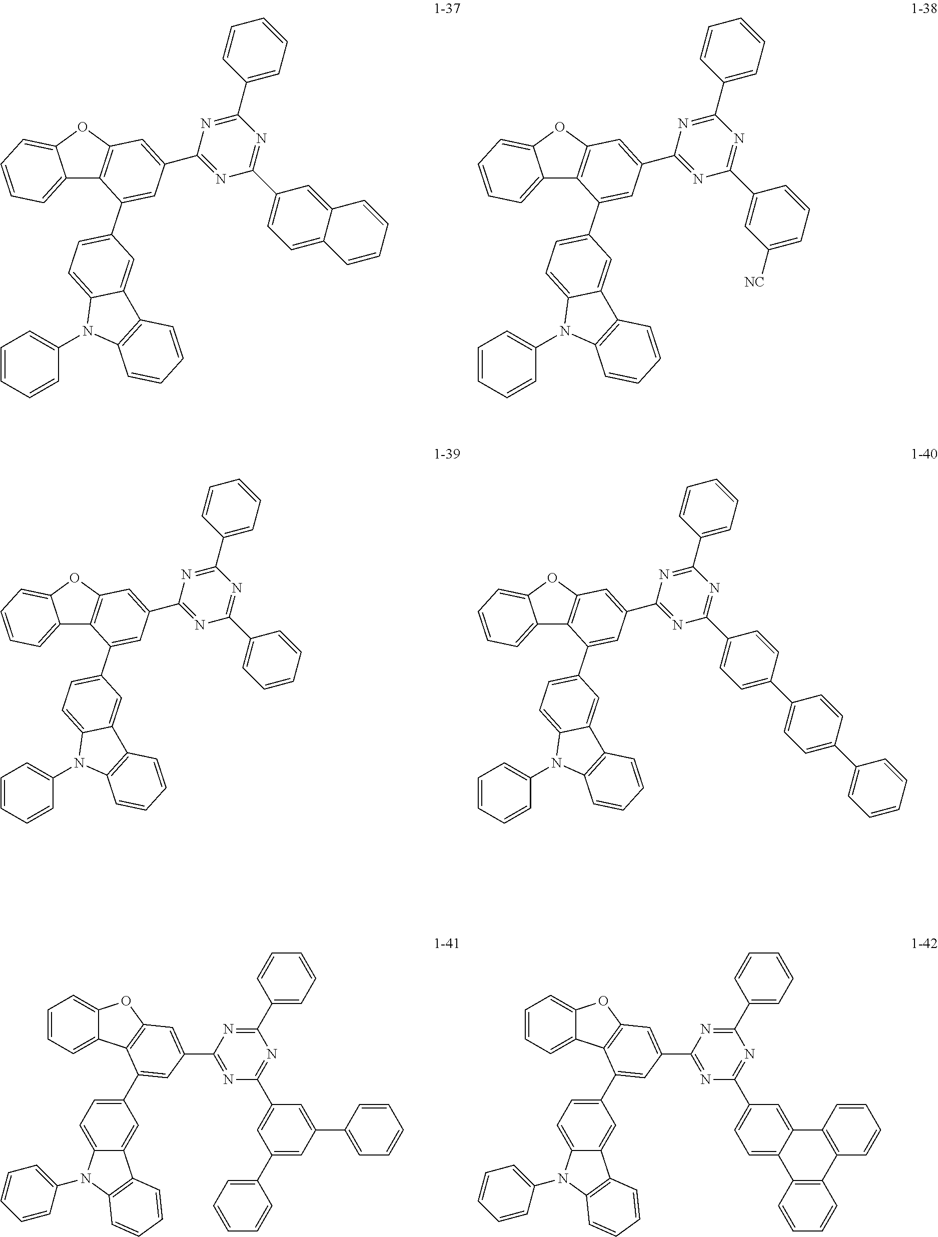

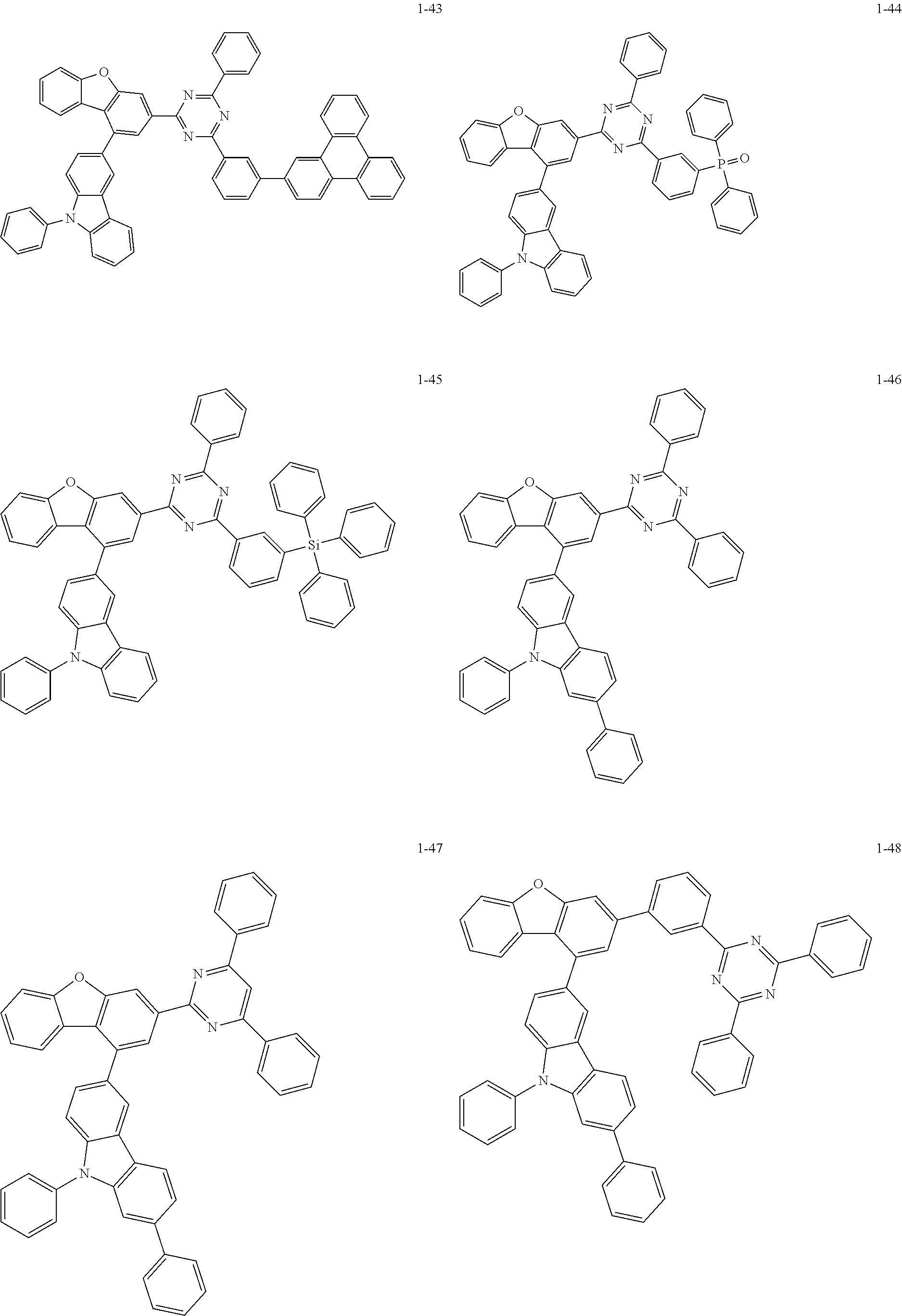

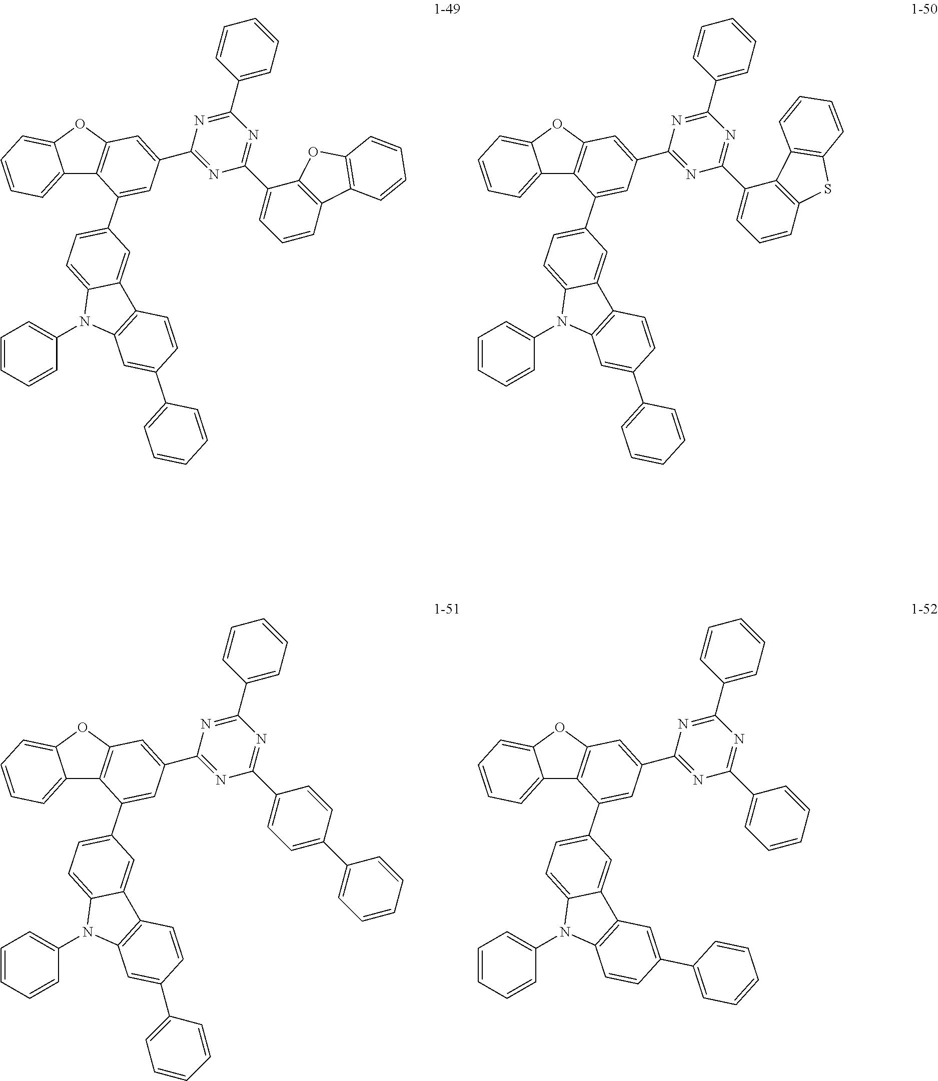 [SMILES: c1ccc(-c2ccc3c4cc(-c5cc(-c6nc(-c7ccccc7)nc(-c7cccc8c7oc7ccccc78)n6)cc6oc7ccccc7c56)ccc4n(-c4ccccc4)c3c2)cc1 ; c1ccc(-c2ccc3c4cc(-c5cc(-c6nc(-c7ccccc7)nc(-c7cccc8sc9ccccc9c78)n6)cc6oc7ccccc7c56)ccc4n(-c4ccccc4)c3c2)cc1 ; c1ccc(-c2ccc(-c3nc(-c4ccccc4)nc(-c4cc(-c5ccc6c(c5)c5ccc(-c7ccccc7)cc5n6-c5ccccc5)c5c(c4)oc4ccccc45)n3)cc2)cc1 ; c1ccc(-c2ccc3c(c2)c2cc(-c4cc(-c5nc(-c6ccccc6)nc(-c6ccccc6)n5)cc5oc6ccccc6c45)ccc2n3-c2ccccc2)cc1]

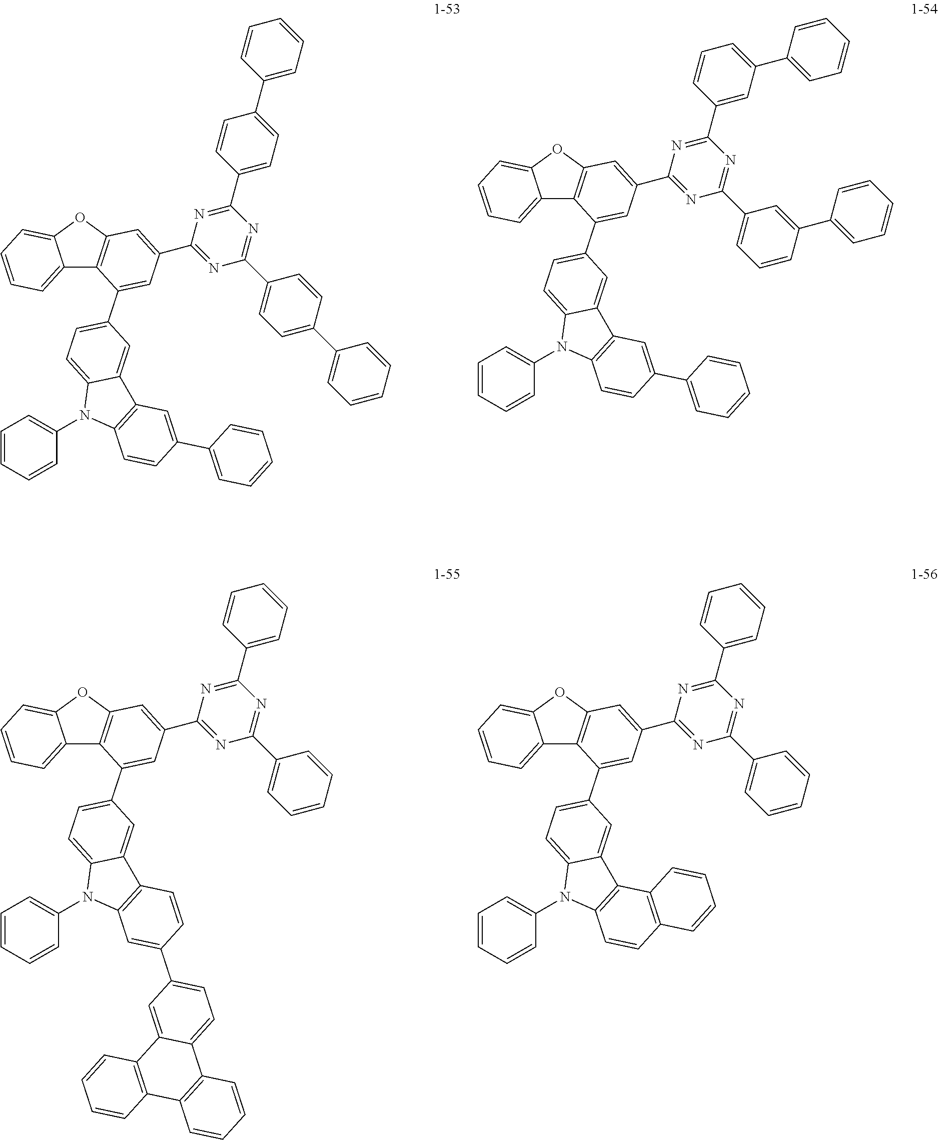 [SMILES: c1ccc(-c2ccc(-c3nc(-c4ccc(-c5ccccc5)cc4)nc(-c4cc(-c5ccc6c(c5)c5cc(-c7ccccc7)ccc5n6-c5ccccc5)c5c(c4)oc4ccccc45)n3)cc2)cc1 ; c1ccc(-c2cccc(-c3nc(-c4cccc(-c5ccccc5)c4)nc(-c4cc(-c5ccc6c(c5)c5cc(-c7ccccc7)ccc5n6-c5ccccc5)c5c(c4)oc4ccccc45)n3)c2)cc1 ; c1ccc(-c2nc(-c3ccccc3)nc(-c3cc(-c4ccc5c(c4)c4ccc(-c6ccc7c8ccccc8c8ccccc8c7c6)cc4n5-c4ccccc4)c4c(c3)oc3ccccc34)n2)cc1 ; c1ccc(-c2nc(-c3ccccc3)nc(-c3cc(-c4ccc5c(c4)c4c6ccccc6ccc4n5-c4ccccc4)c4c(c3)oc3ccccc34)n2)cc1]

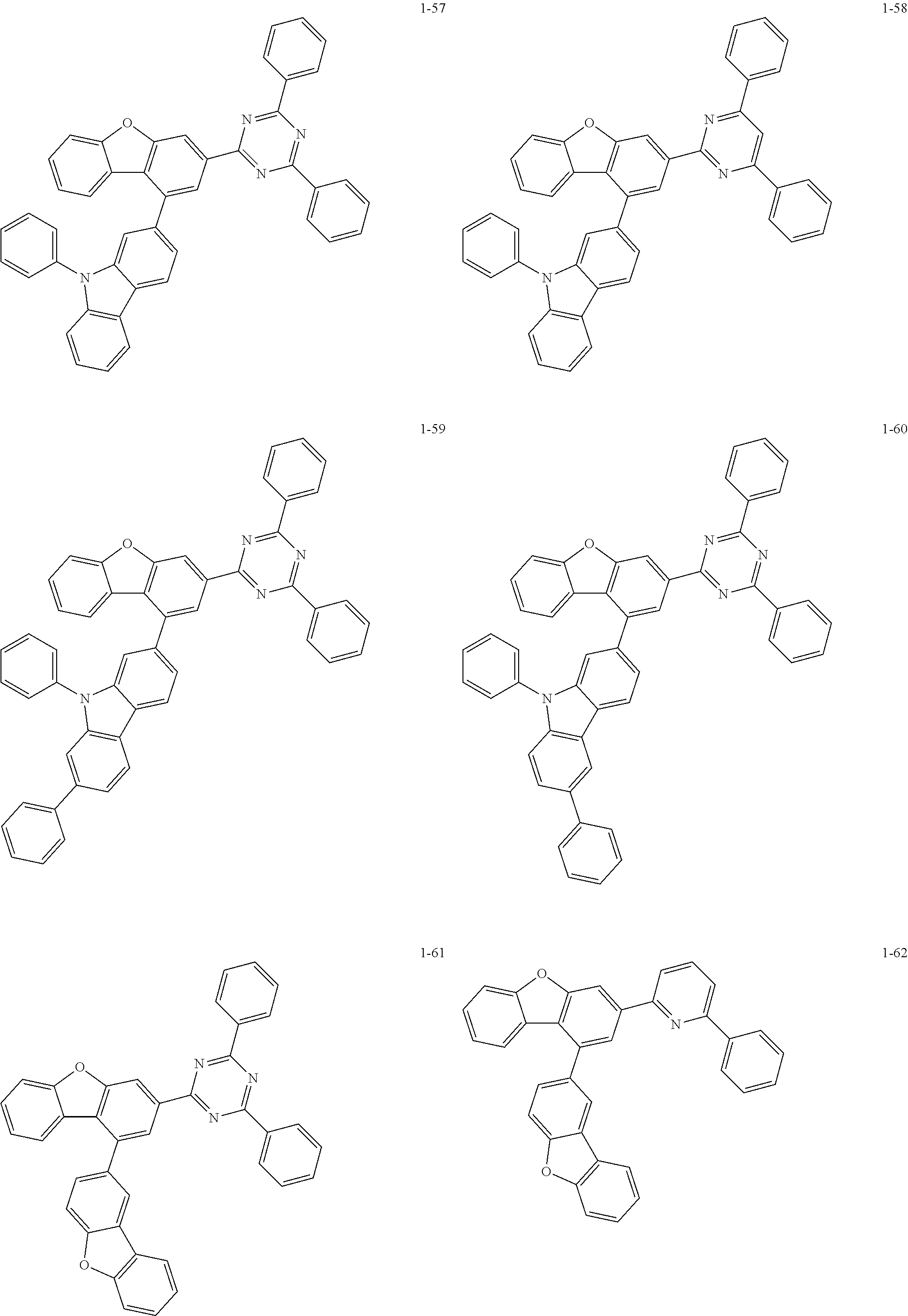

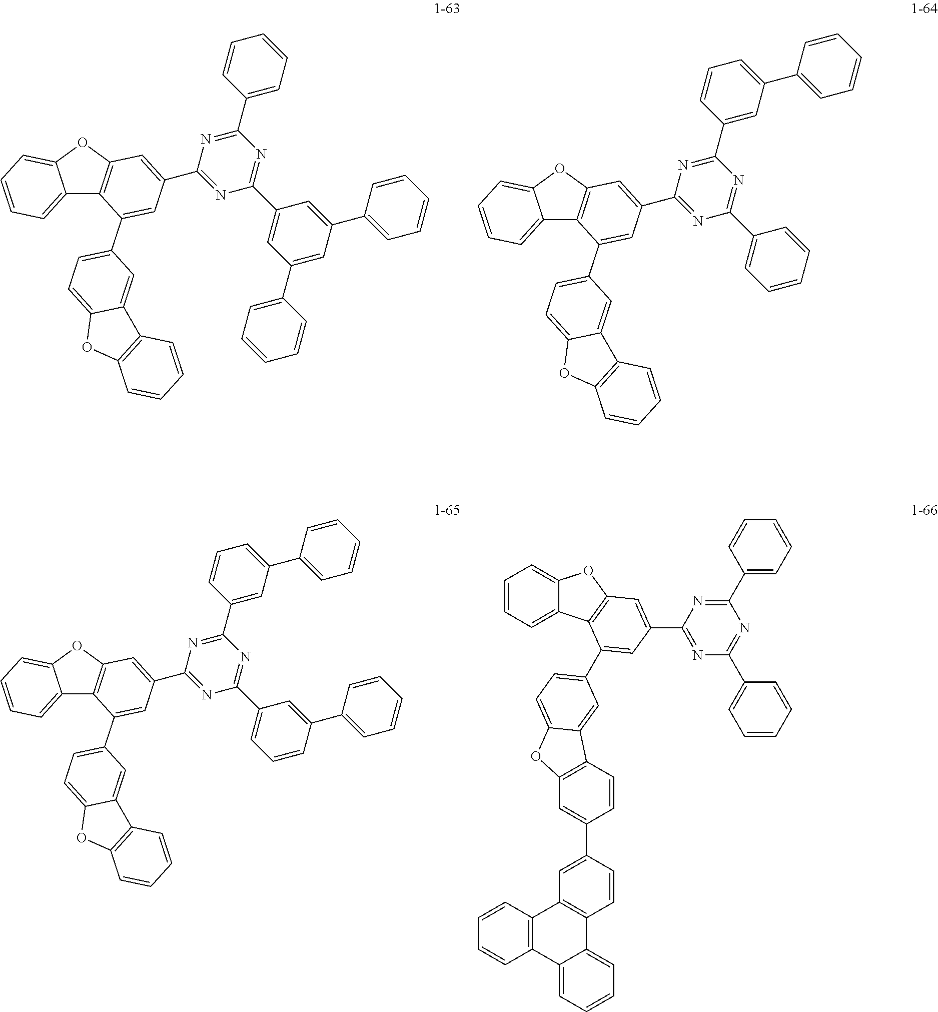 [SMILES: c1ccc(-c2cc(-c3ccccc3)cc(-c3nc(-c4ccccc4)nc(-c4cc(-c5ccc6oc7ccccc7c6c5)c5c(c4)oc4ccccc45)n3)c2)cc1 ; c1ccc(-c2cccc(-c3nc(-c4ccccc4)nc(-c4cc(-c5ccc6oc7ccccc7c6c5)c5c(c4)oc4ccccc45)n3)c2)cc1 ; c1ccc(-c2cccc(-c3nc(-c4cccc(-c5ccccc5)c4)nc(-c4cc(-c5ccc6oc7ccccc7c6c5)c5c(c4)oc4ccccc45)n3)c2)cc1 ; c1ccc(-c2nc(-c3ccccc3)nc(-c3cc(-c4ccc5oc6cc(-c7ccc8c9ccccc9c9ccccc9c8c7)ccc6c5c4)c4c(c3)oc3ccccc34)n2)cc1]

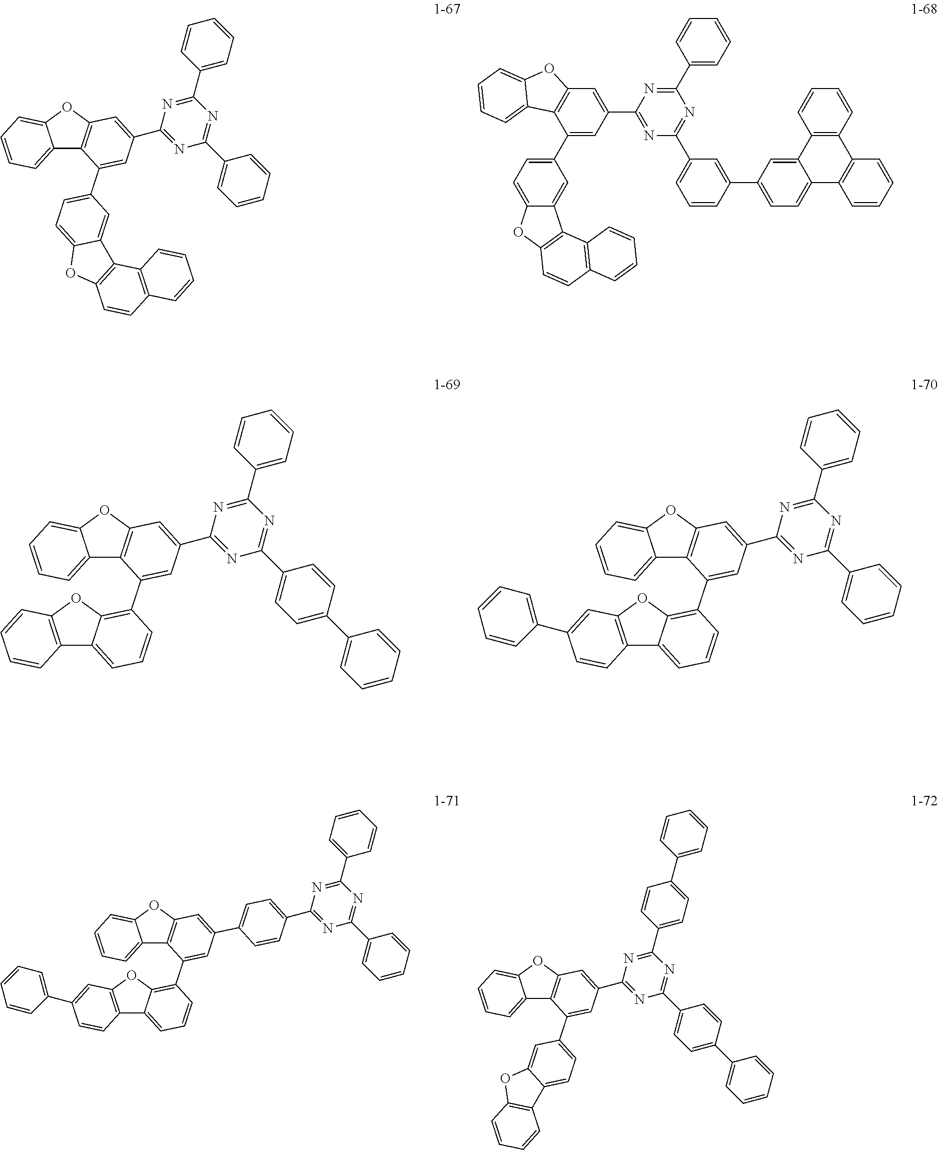

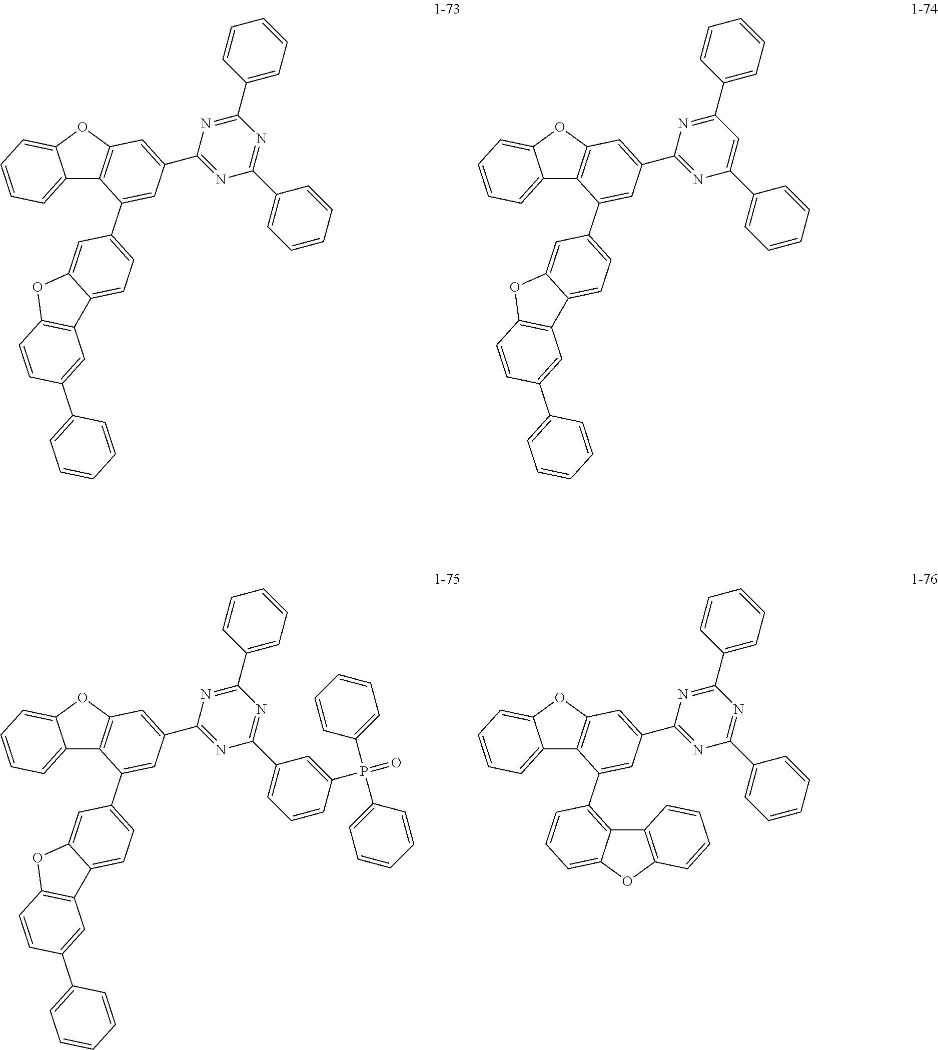 [SMILES: c1ccc(-c2ccc3oc4cc(-c5cc(-c6nc(-c7ccccc7)nc(-c7ccccc7)n6)cc6oc7ccccc7c56)ccc4c3c2)cc1 ; c1ccc(-c2ccc3oc4cc(-c5cc(-c6nc(-c7ccccc7)cc(-c7ccccc7)n6)cc6oc7ccccc7c56)ccc4c3c2)cc1 ; O=P(c1ccccc1)(c1ccccc1)c1cccc(-c2nc(-c3ccccc3)nc(-c3cc(-c4ccc5c(c4)oc4ccc(-c6ccccc6)cc45)c4c(c3)oc3ccccc34)n2)c1 ; c1ccc(-c2nc(-c3ccccc3)nc(-c3cc(-c4cccc5oc6ccccc6c45)c4c(c3)oc3ccccc34)n2)cc1]

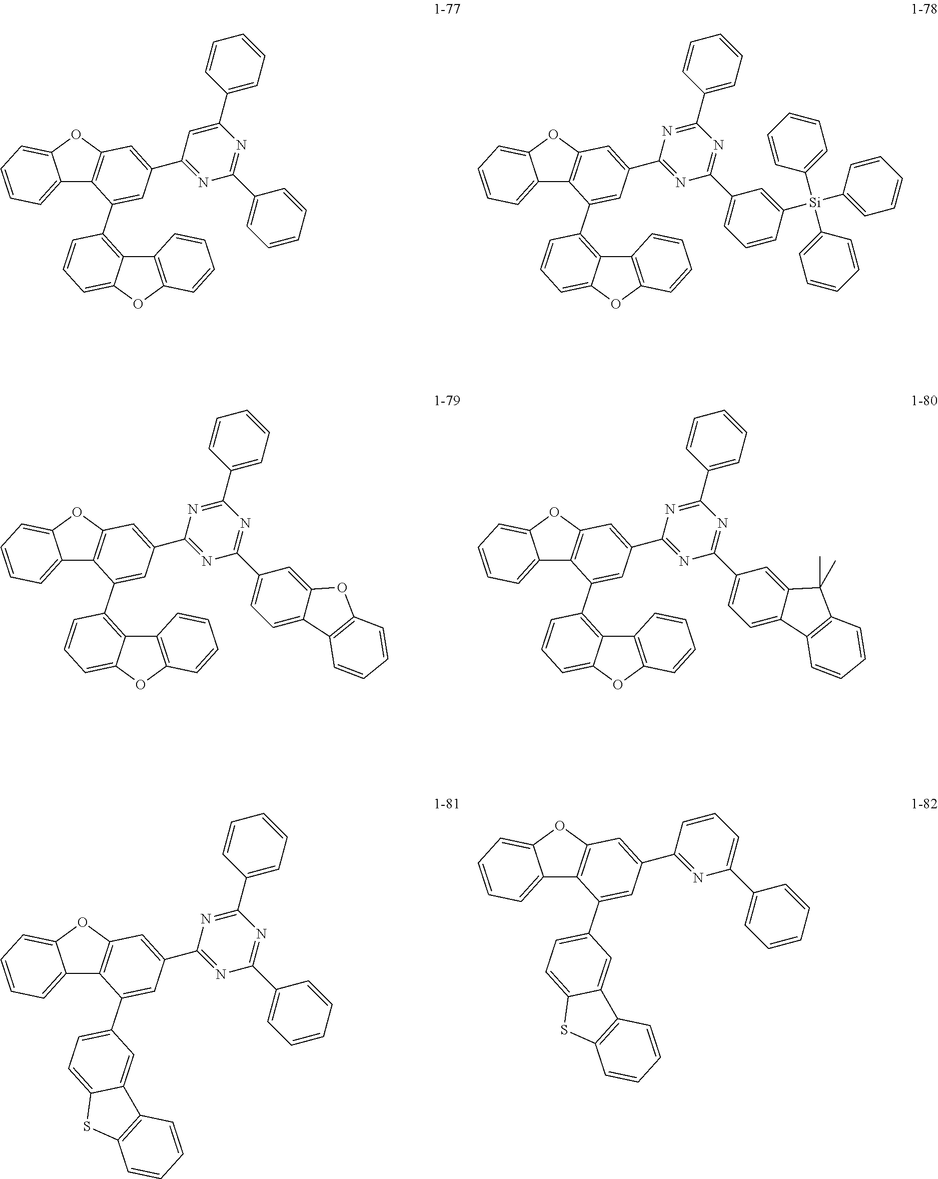

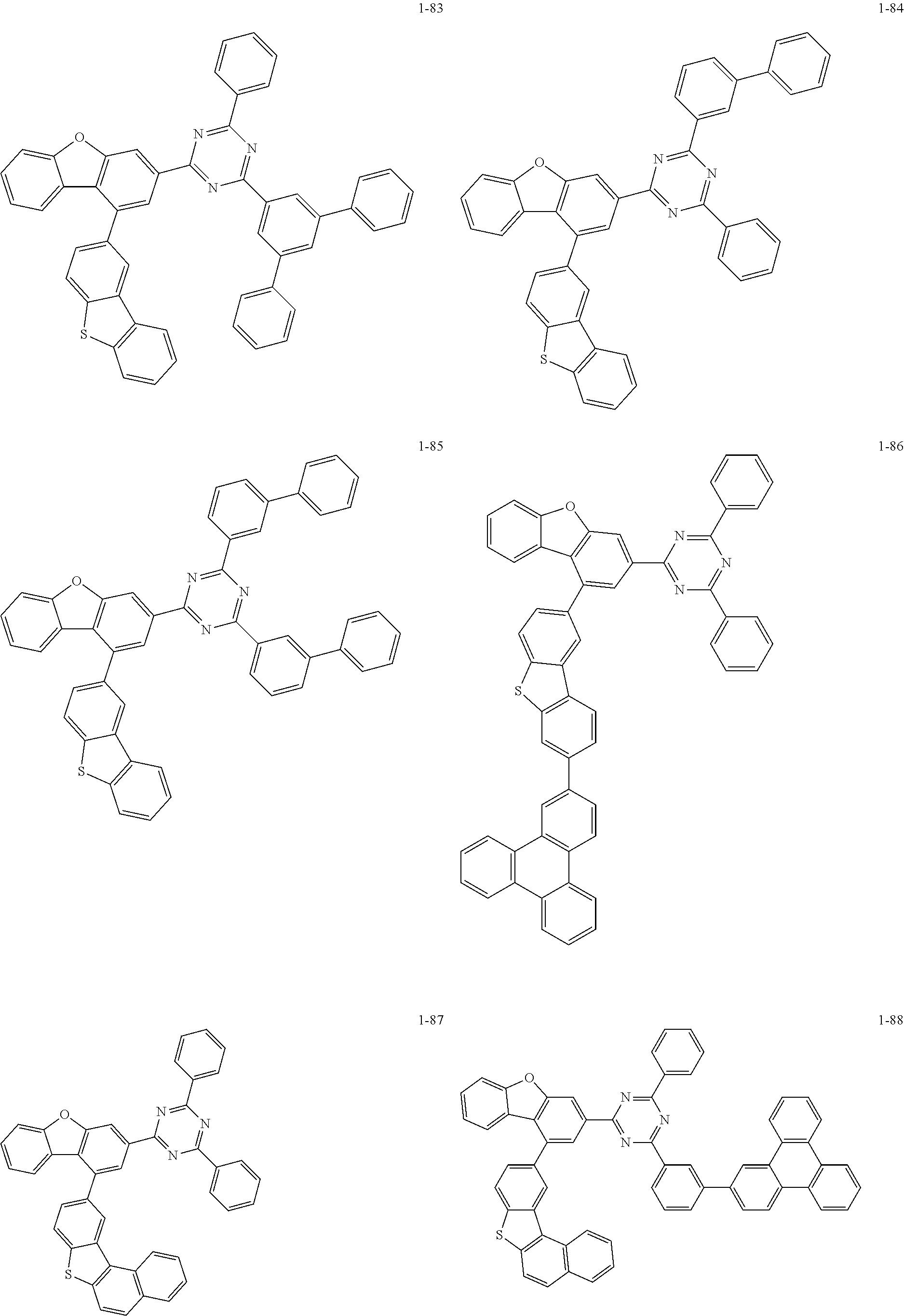

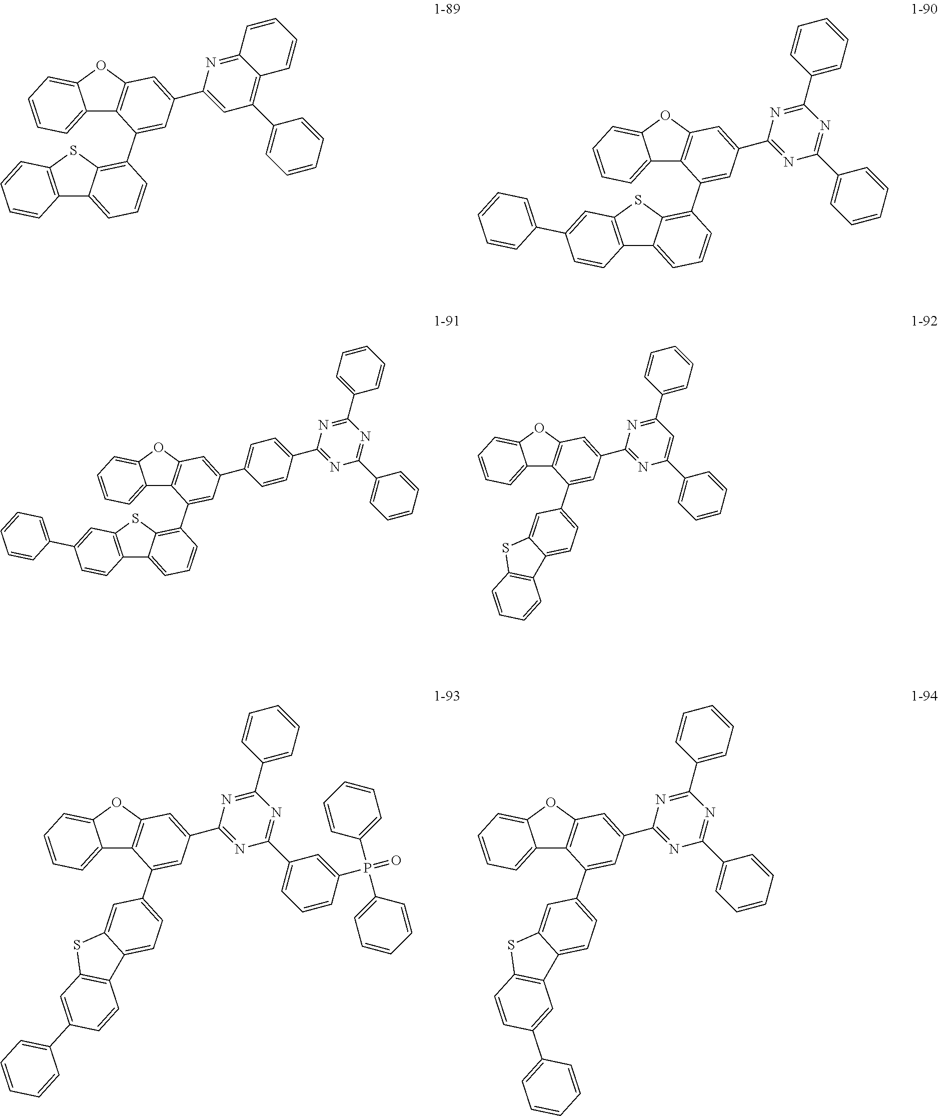

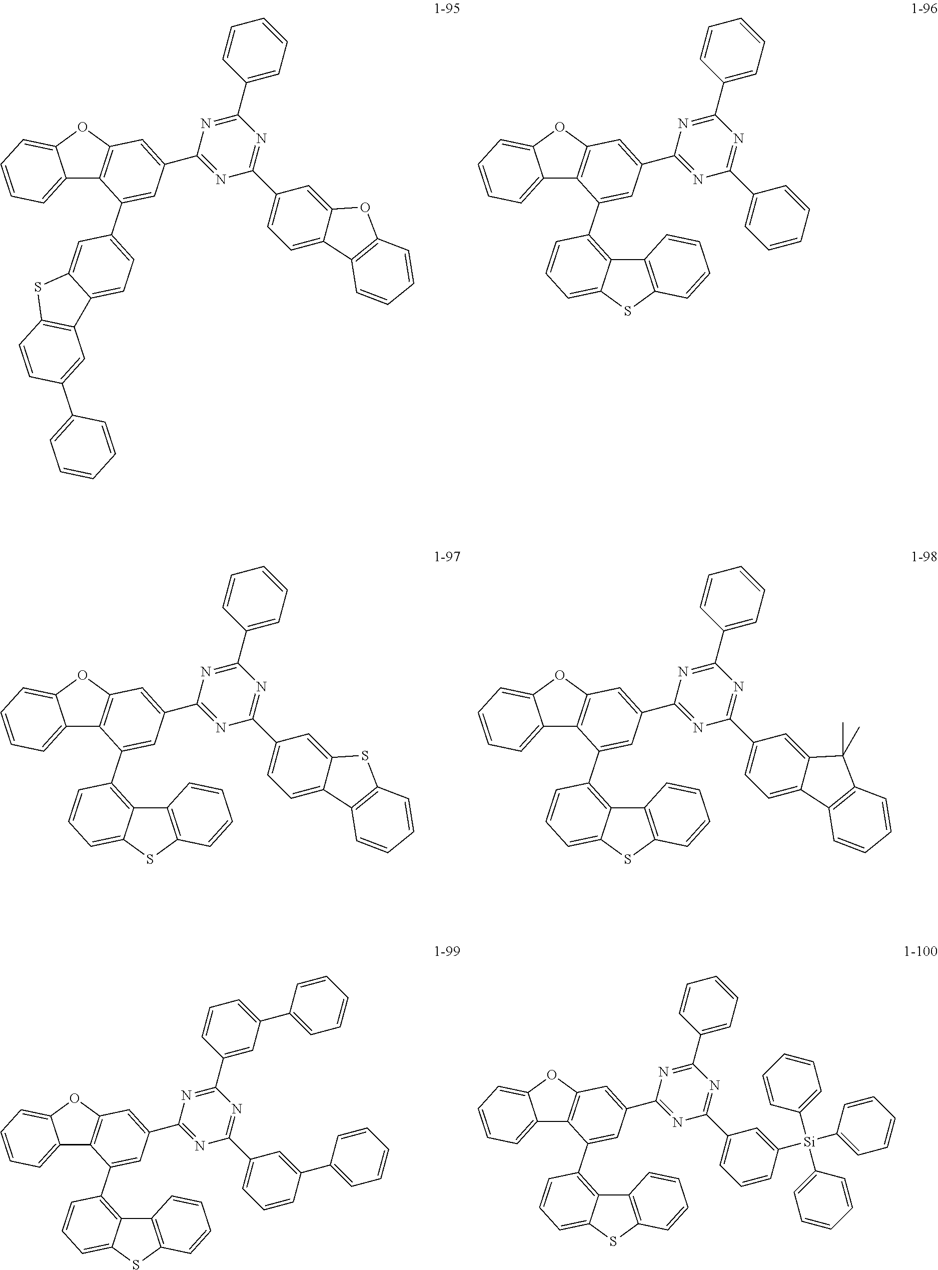

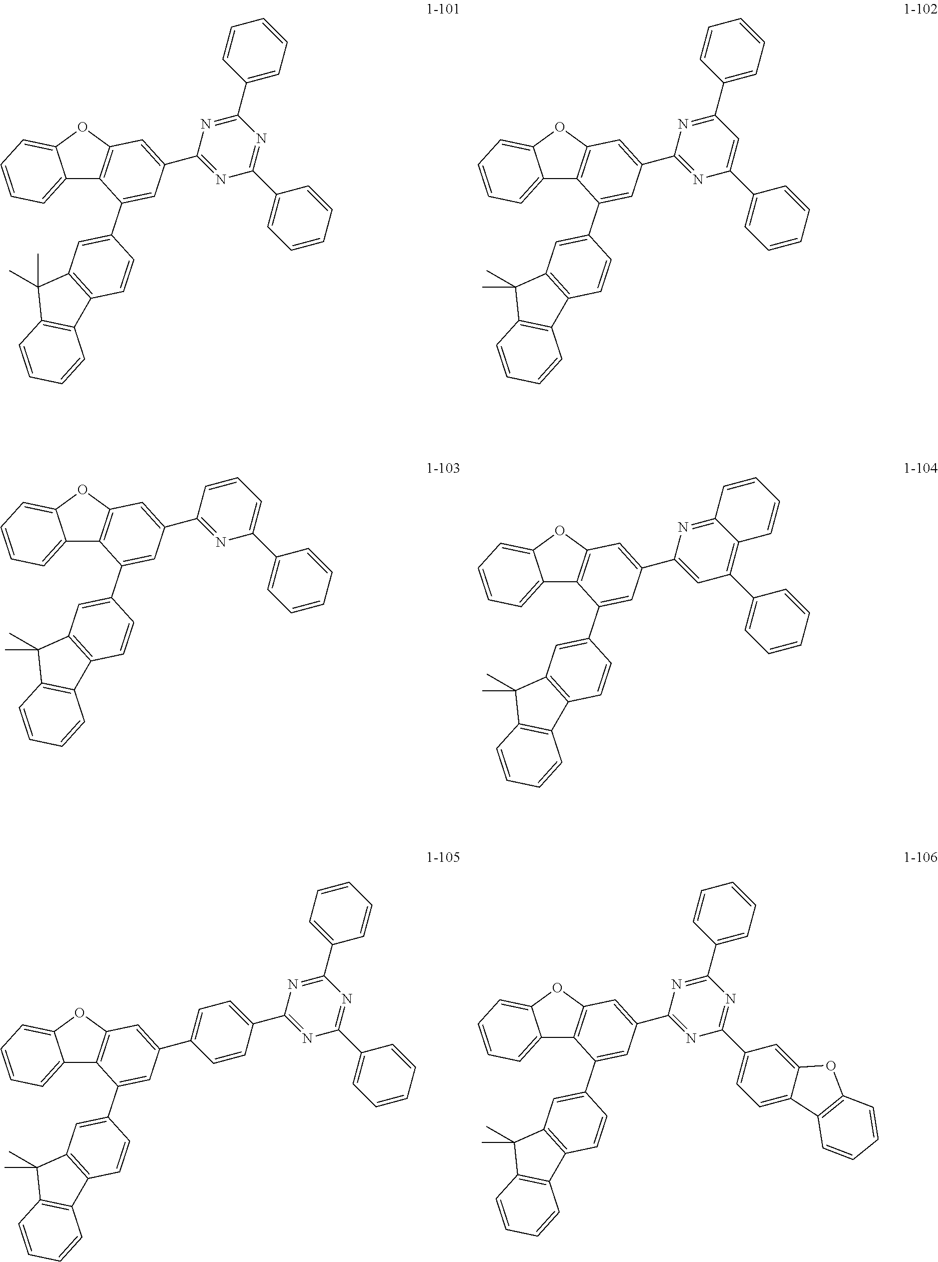 [SMILES: CC1(C)c2ccccc2-c2ccc(-c3cc(-c4nc(-c5ccccc5)nc(-c5ccccc5)n4)cc4oc5ccccc5c34)cc21 ; CC1(C)c2ccccc2-c2ccc(-c3cc(-c4nc(-c5ccccc5)cc(-c5ccccc5)n4)cc4oc5ccccc5c34)cc21 ; CC1(C)c2ccccc2-c2ccc(-c3cc(-c4cccc(-c5ccccc5)n4)cc4oc5ccccc5c34)cc21 ; CC1(C)c2ccccc2-c2ccc(-c3cc(-c4cc(-c5ccccc5)c5ccccc5n4)cc4oc5ccccc5c34)cc21 ; CC1(C)c2ccccc2-c2ccc(-c3cc(-c4ccc(-c5nc(-c6ccccc6)nc(-c6ccccc6)n5)cc4)cc4oc5ccccc5c34)cc21 ; CC1(C)c2ccccc2-c2ccc(-c3cc(-c4nc(-c5ccccc5)nc(-c5ccc6c(c5)oc5ccccc56)n4)cc4oc5ccccc5c34)cc21]

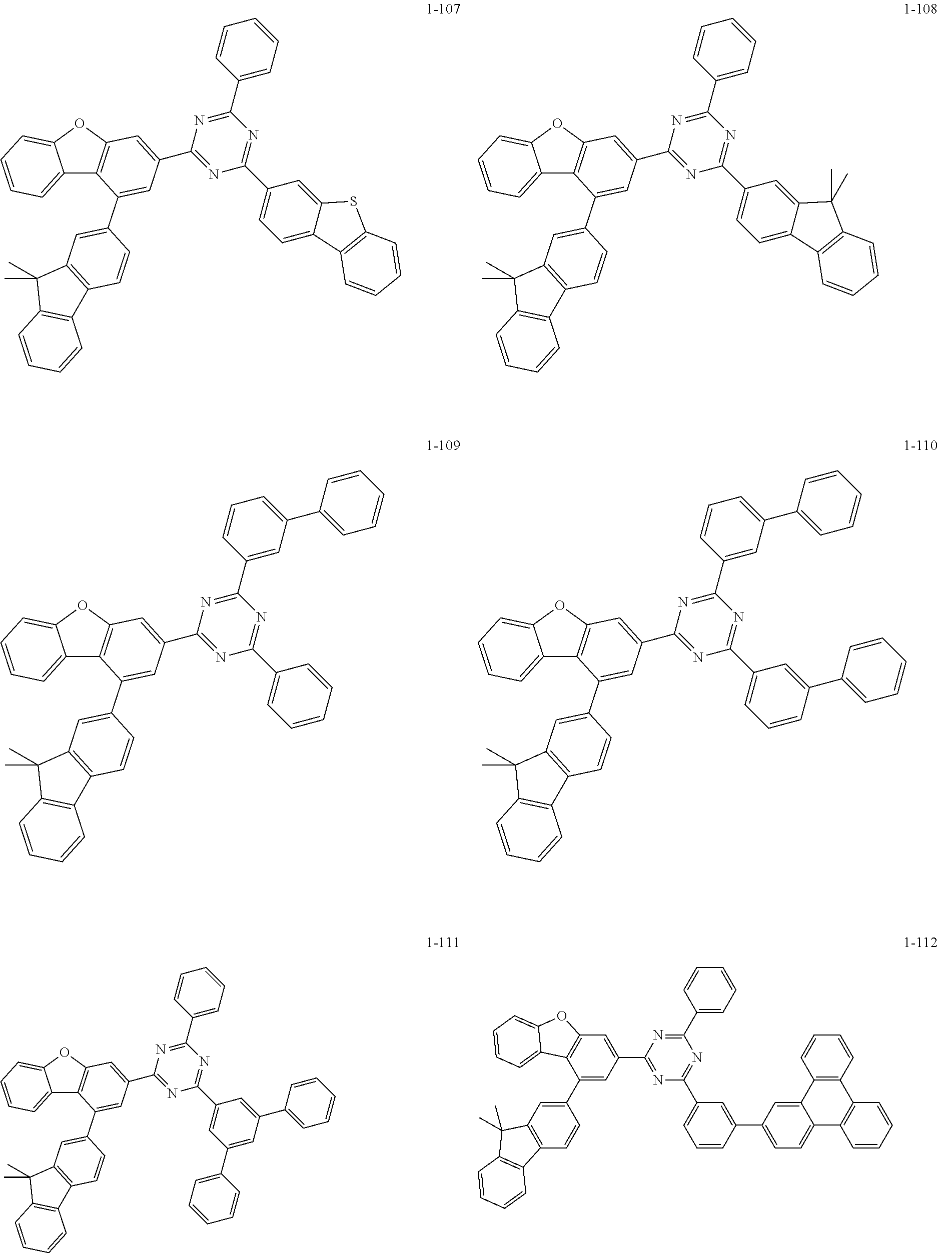

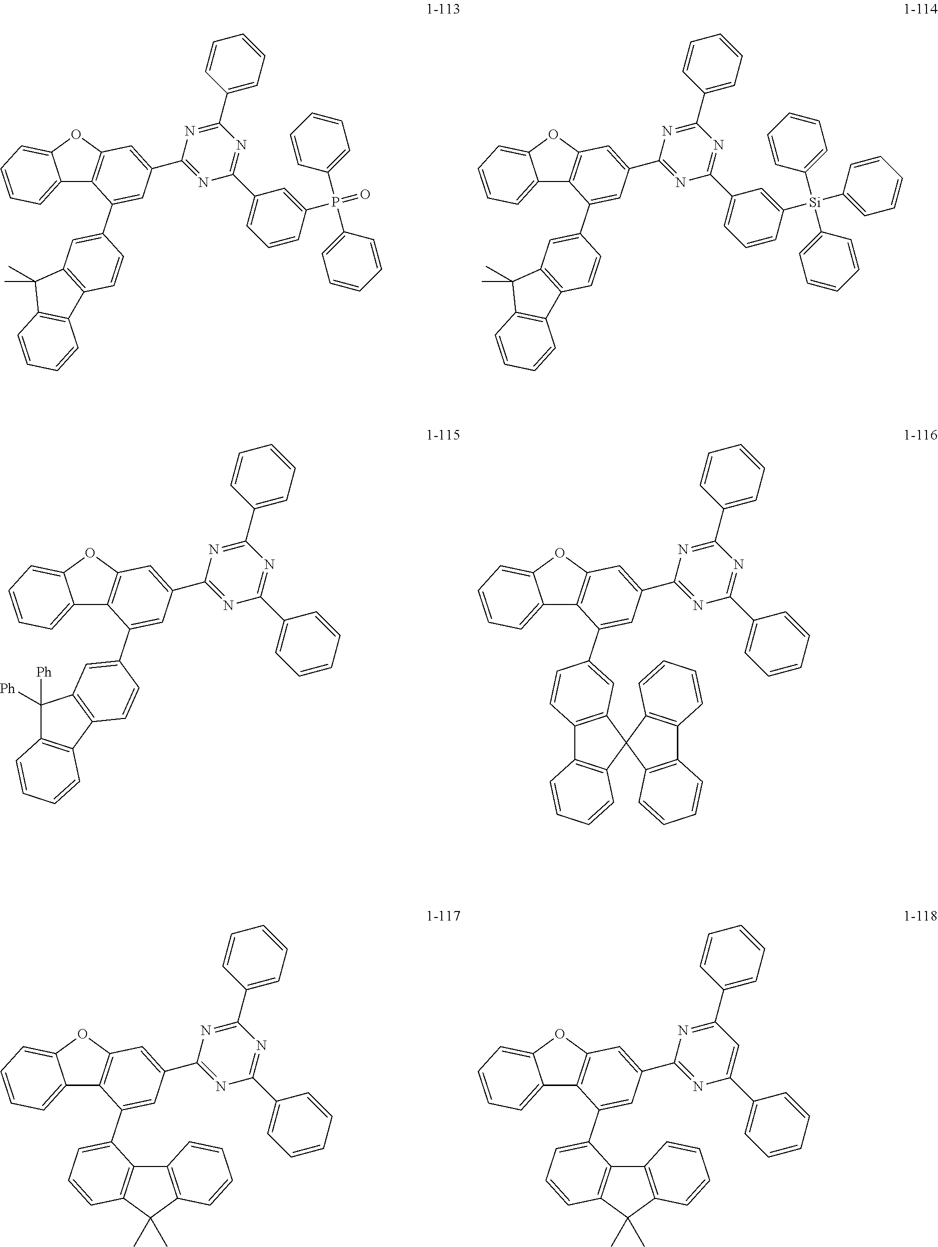

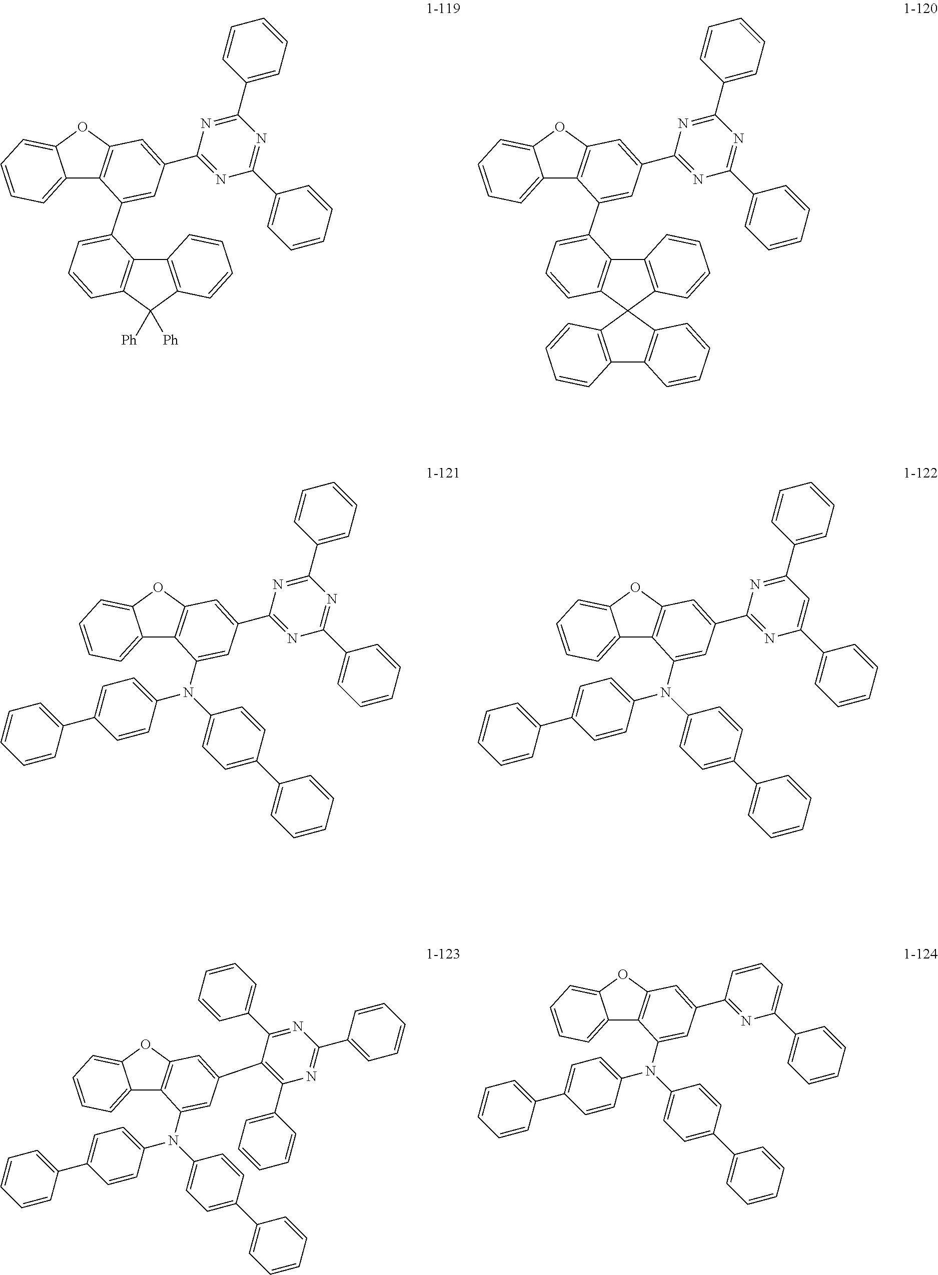

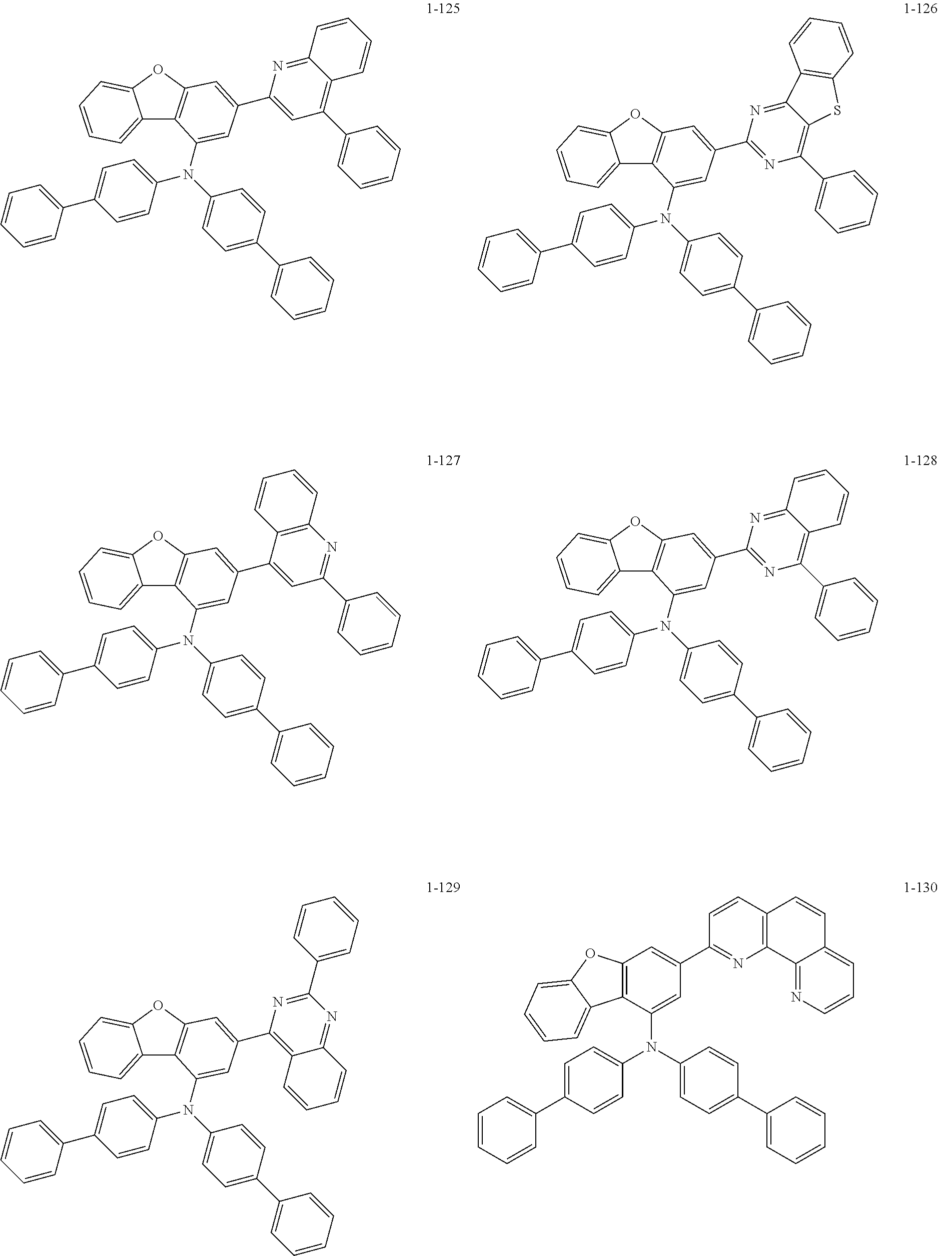

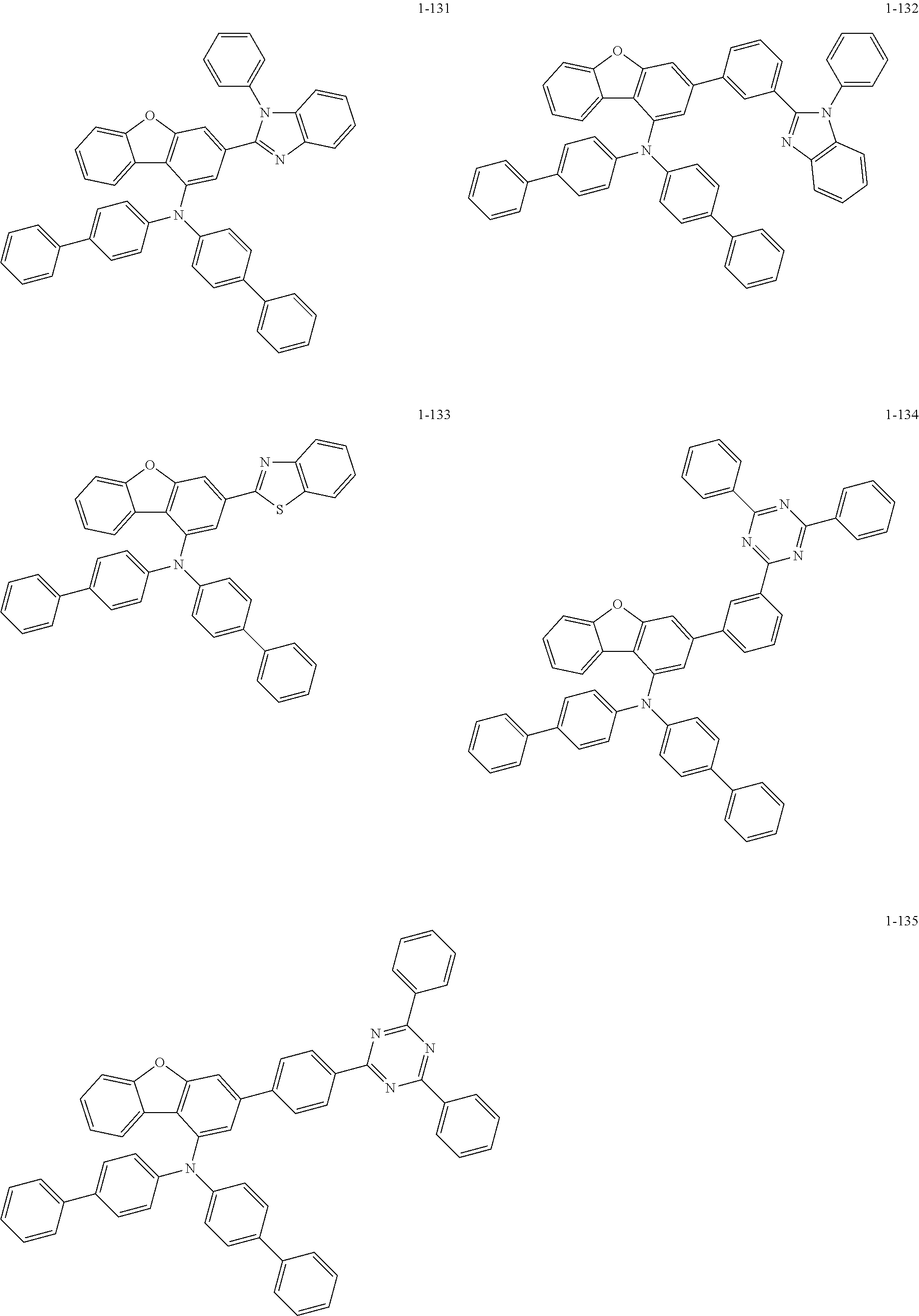 [SMILES: c1ccc(-c2ccc(N(c3ccc(-c4ccccc4)cc3)c3cc(-c4nc5ccccc5n4-c4ccccc4)cc4oc5ccccc5c34)cc2)cc1 ; c1ccc(-c2ccc(N(c3ccc(-c4ccccc4)cc3)c3cc(-c4cccc(-c5nc6ccccc6n5-c5ccccc5)c4)cc4oc5ccccc5c34)cc2)cc1 ; c1ccc(-c2ccc(N(c3ccc(-c4ccccc4)cc3)c3cc(-c4nc5ccccc5s4)cc4oc5ccccc5c34)cc2)cc1 ; c1ccc(-c2ccc(N(c3ccc(-c4ccccc4)cc3)c3cc(-c4cccc(-c5nc(-c6ccccc6)nc(-c6ccccc6)n5)c4)cc4oc5ccccc5c34)cc2)cc1 ; c1ccc(-c2ccc(N(c3ccc(-c4ccccc4)cc3)c3cc(-c4ccc(-c5nc(-c6ccccc6)nc(-c6ccccc6)n5)cc4)cc4oc5ccccc5c34)cc2)cc1]

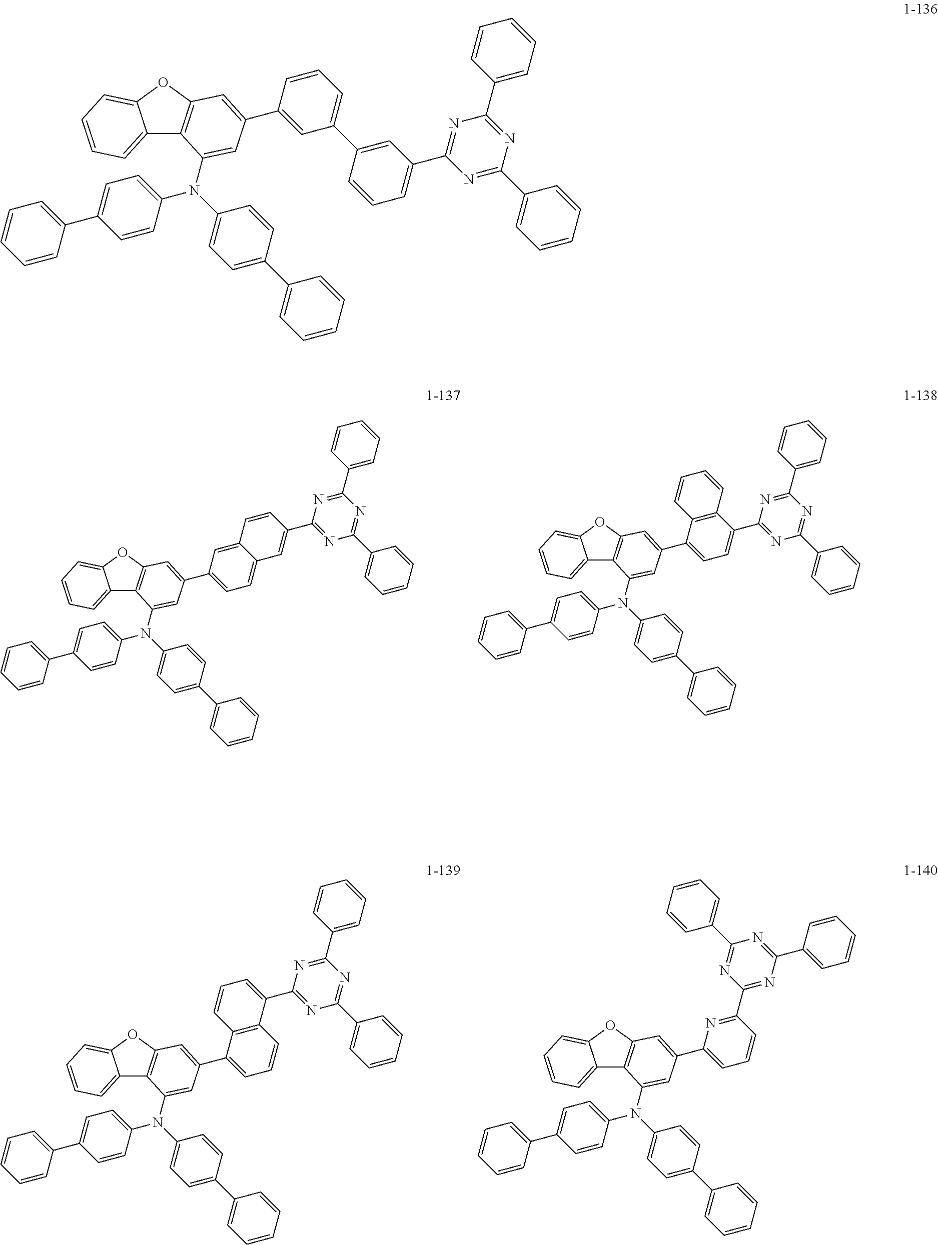

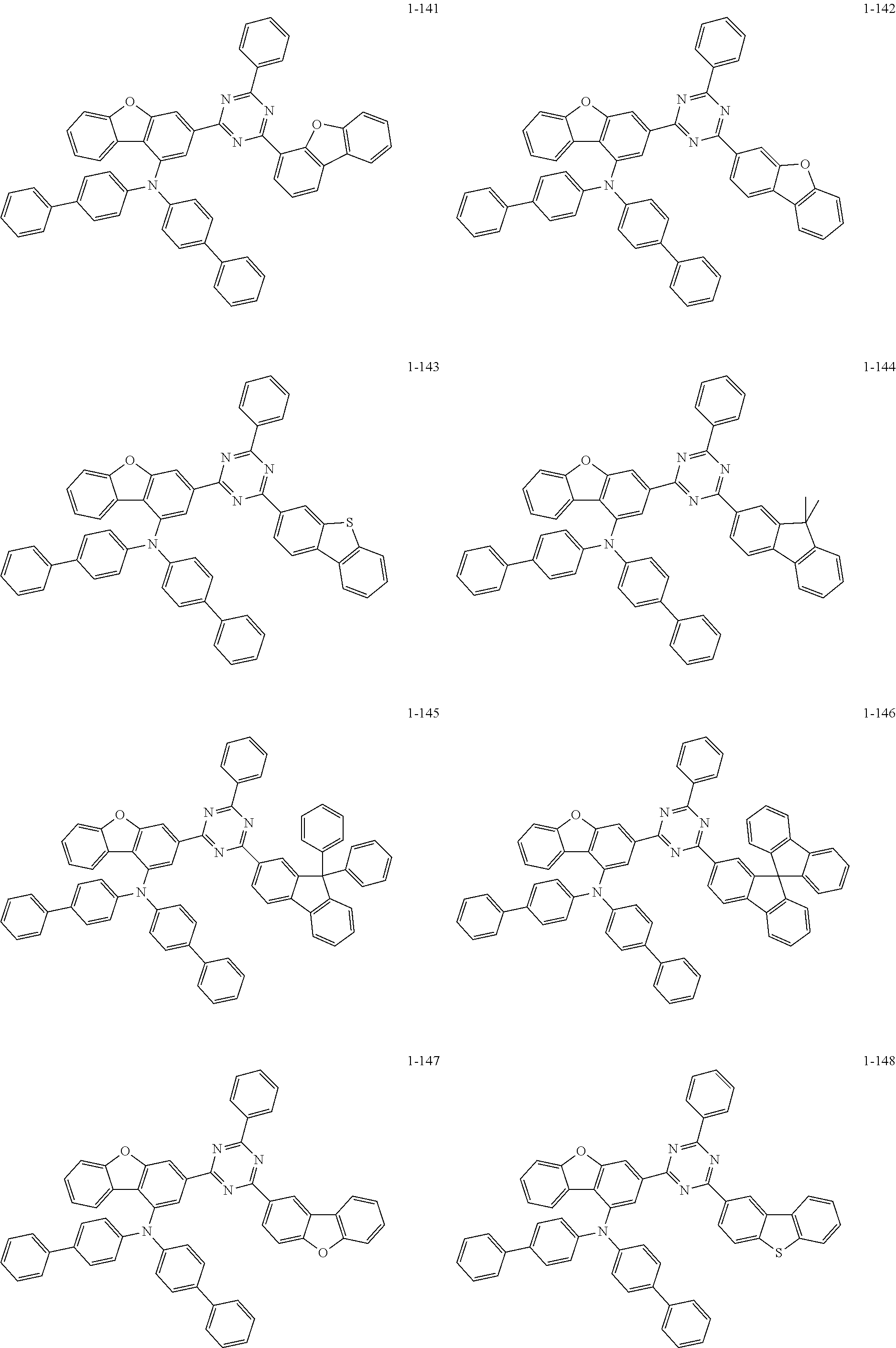

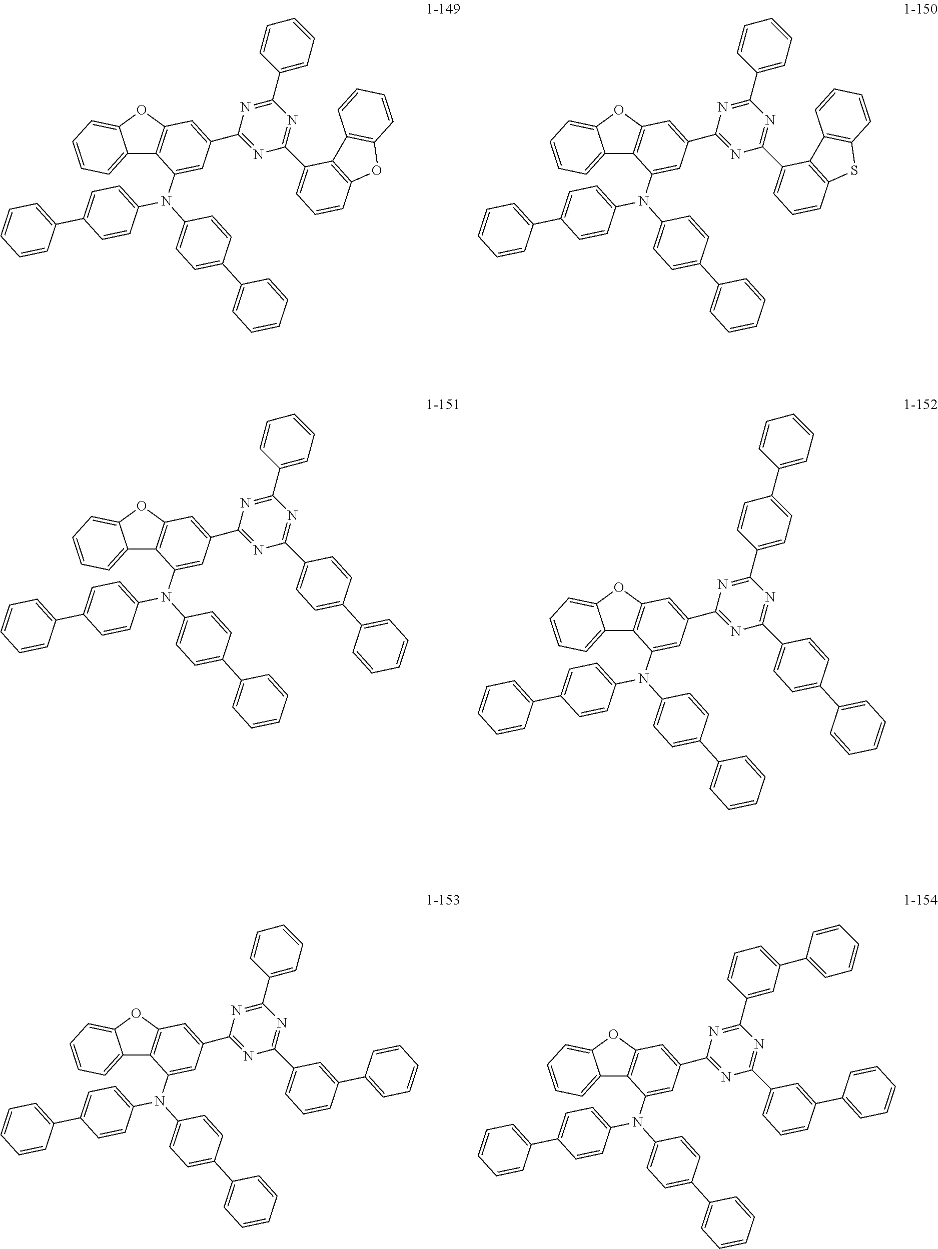

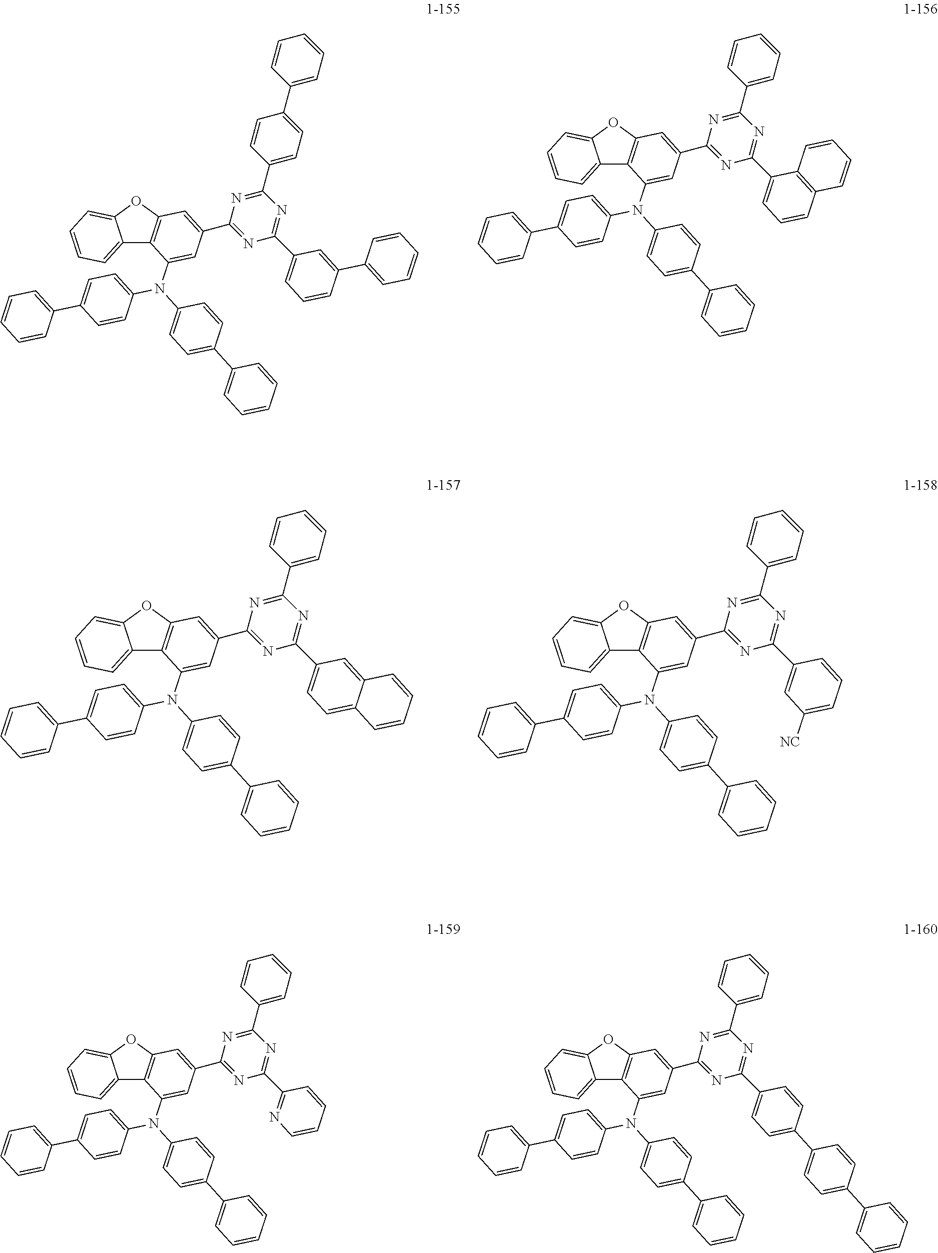

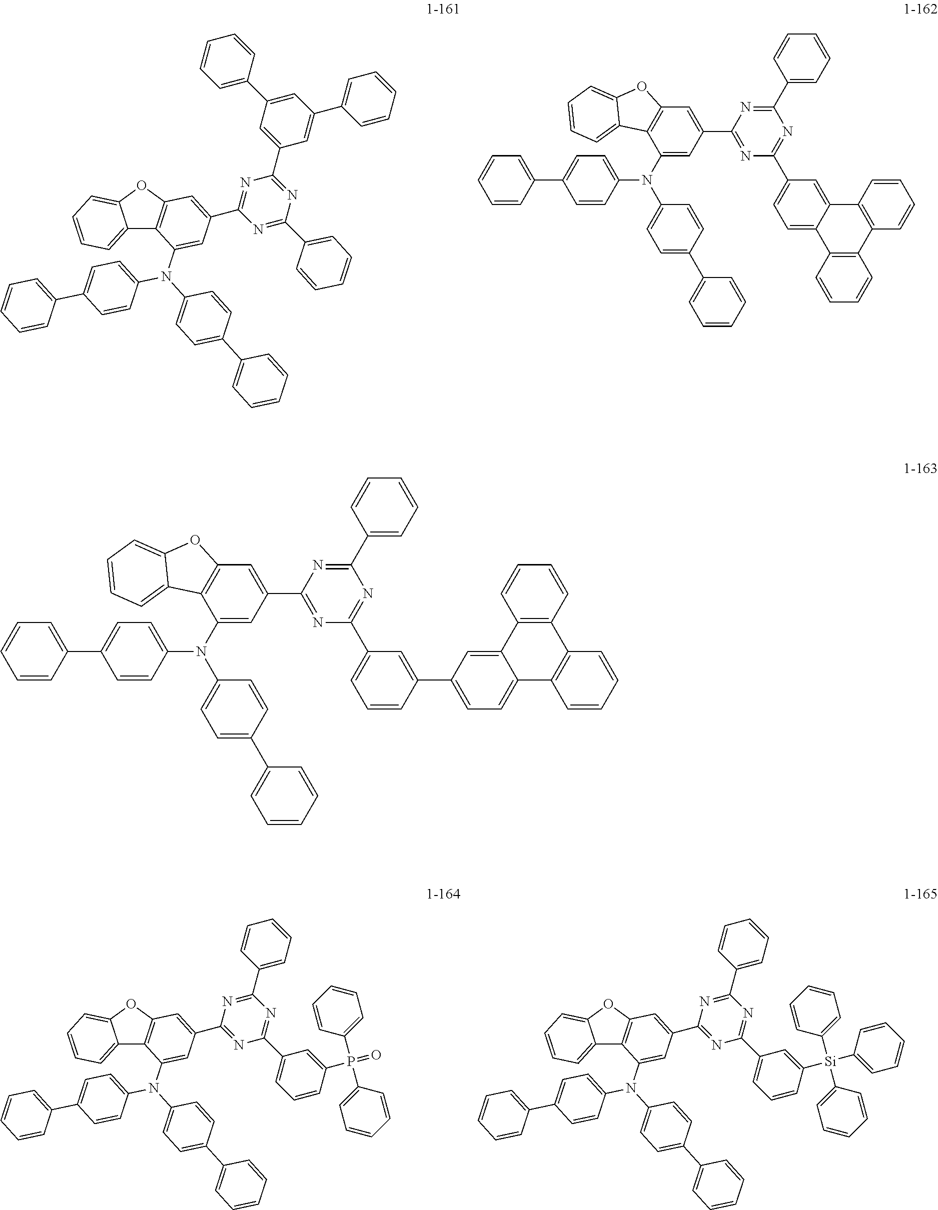

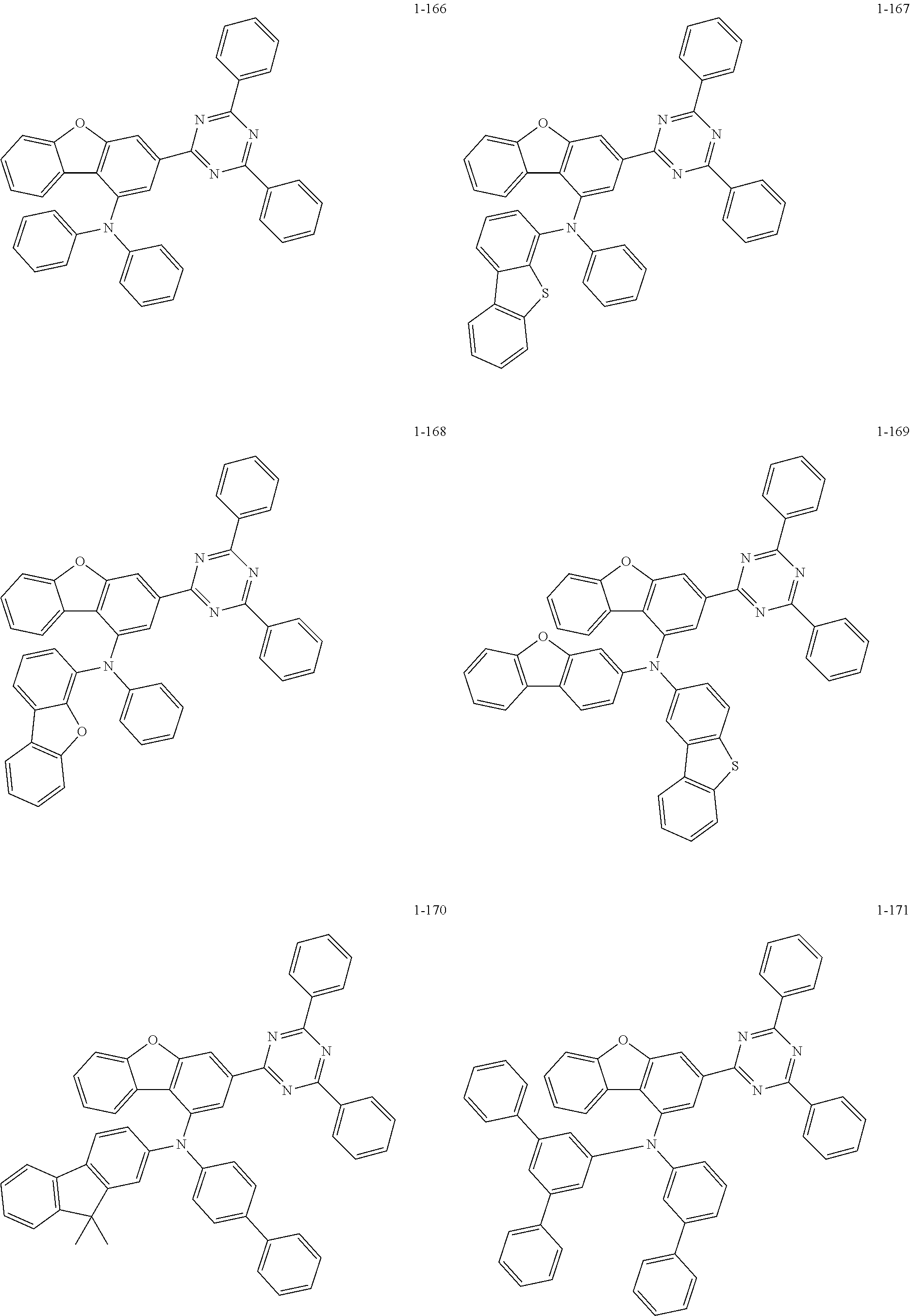

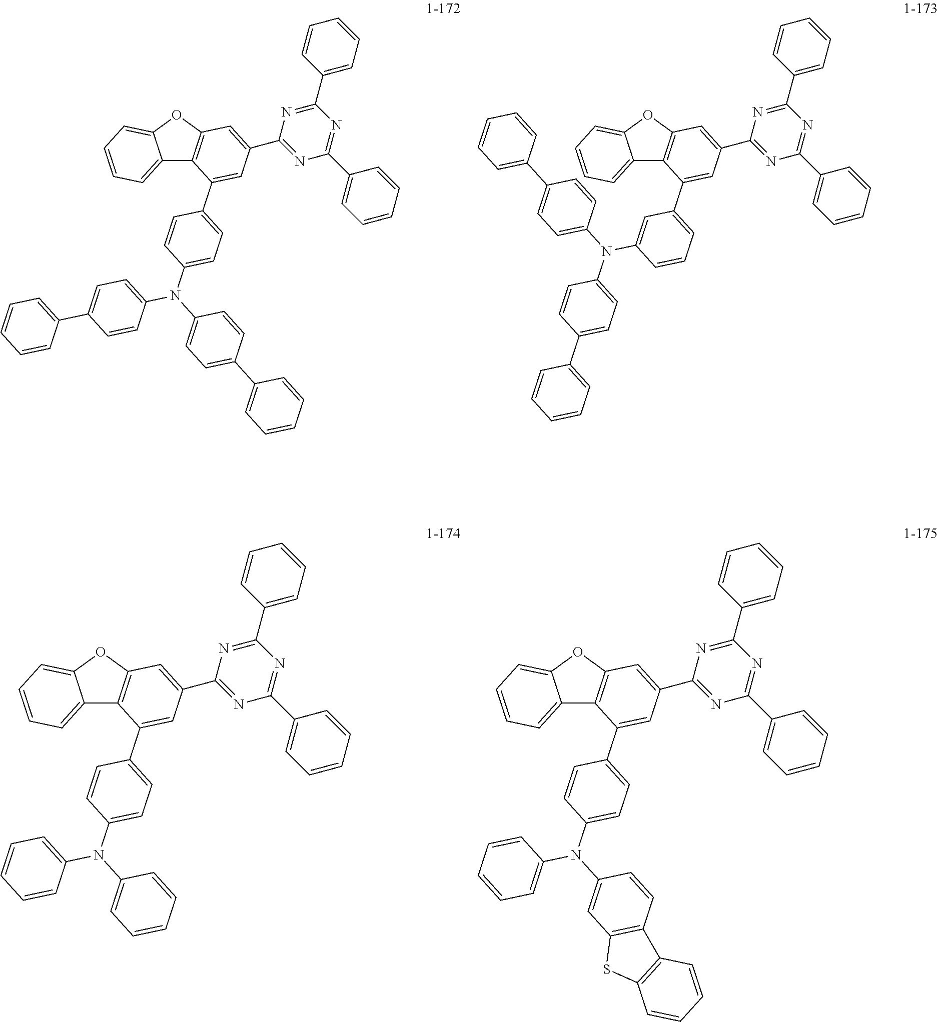 [SMILES: c1ccc(-c2ccc(N(c3ccc(-c4ccccc4)cc3)c3ccc(-c4cc(-c5nc(-c6ccccc6)nc(-c6ccccc6)n5)cc5oc6ccccc6c45)cc3)cc2)cc1 ; c1ccc(-c2ccc(N(c3ccc(-c4ccccc4)cc3)c3cccc(-c4cc(-c5nc(-c6ccccc6)nc(-c6ccccc6)n5)cc5oc6ccccc6c45)c3)cc2)cc1 ; c1ccc(-c2nc(-c3ccccc3)nc(-c3cc(-c4ccc(N(c5ccccc5)c5ccccc5)cc4)c4c(c3)oc3ccccc34)n2)cc1 ; c1ccc(-c2nc(-c3ccccc3)nc(-c3cc(-c4ccc(N(c5ccccc5)c5ccc6c(c5)sc5ccccc56)cc4)c4c(c3)oc3ccccc34)n2)cc1]

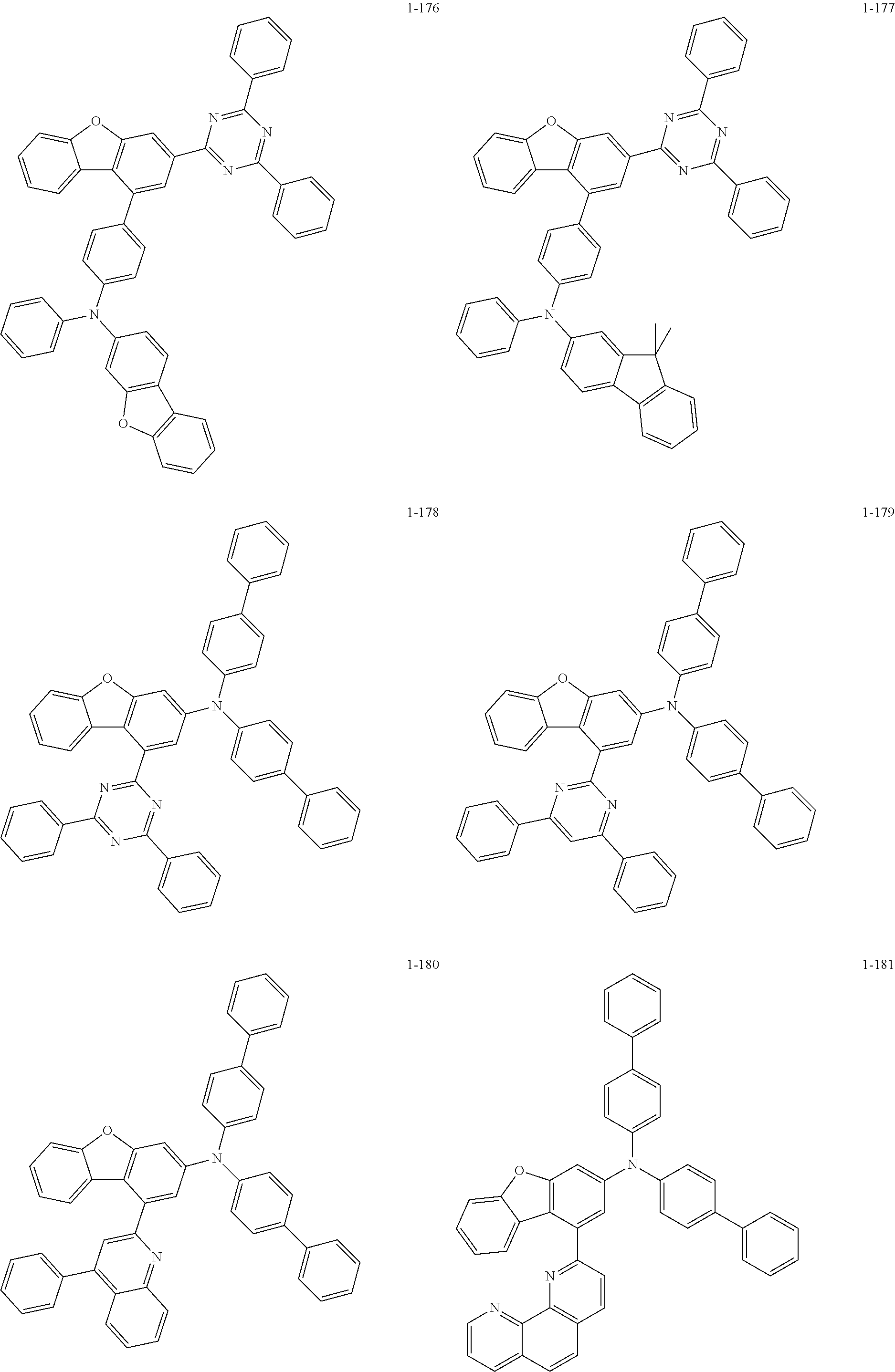

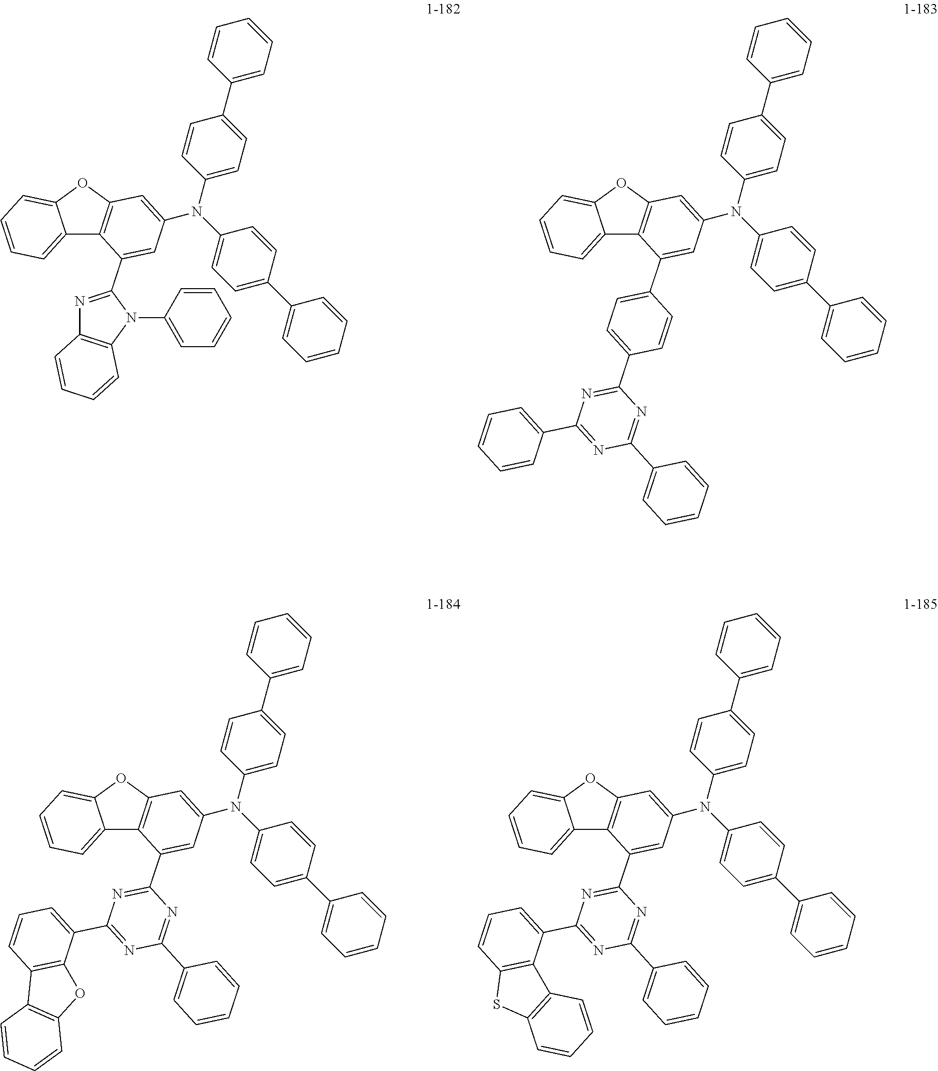 [SMILES: c1ccc(-c2ccc(N(c3ccc(-c4ccccc4)cc3)c3cc(-c4nc5ccccc5n4-c4ccccc4)c4c(c3)oc3ccccc34)cc2)cc1 ; c1ccc(-c2ccc(N(c3ccc(-c4ccccc4)cc3)c3cc(-c4ccc(-c5nc(-c6ccccc6)nc(-c6ccccc6)n5)cc4)c4c(c3)oc3ccccc34)cc2)cc1 ; c1ccc(-c2ccc(N(c3ccc(-c4ccccc4)cc3)c3cc(-c4nc(-c5ccccc5)nc(-c5cccc6c5oc5ccccc56)n4)c4c(c3)oc3ccccc34)cc2)cc1 ; c1ccc(-c2ccc(N(c3ccc(-c4ccccc4)cc3)c3cc(-c4nc(-c5ccccc5)nc(-c5cccc6sc7ccccc7c56)n4)c4c(c3)oc3ccccc34)cc2)cc1]

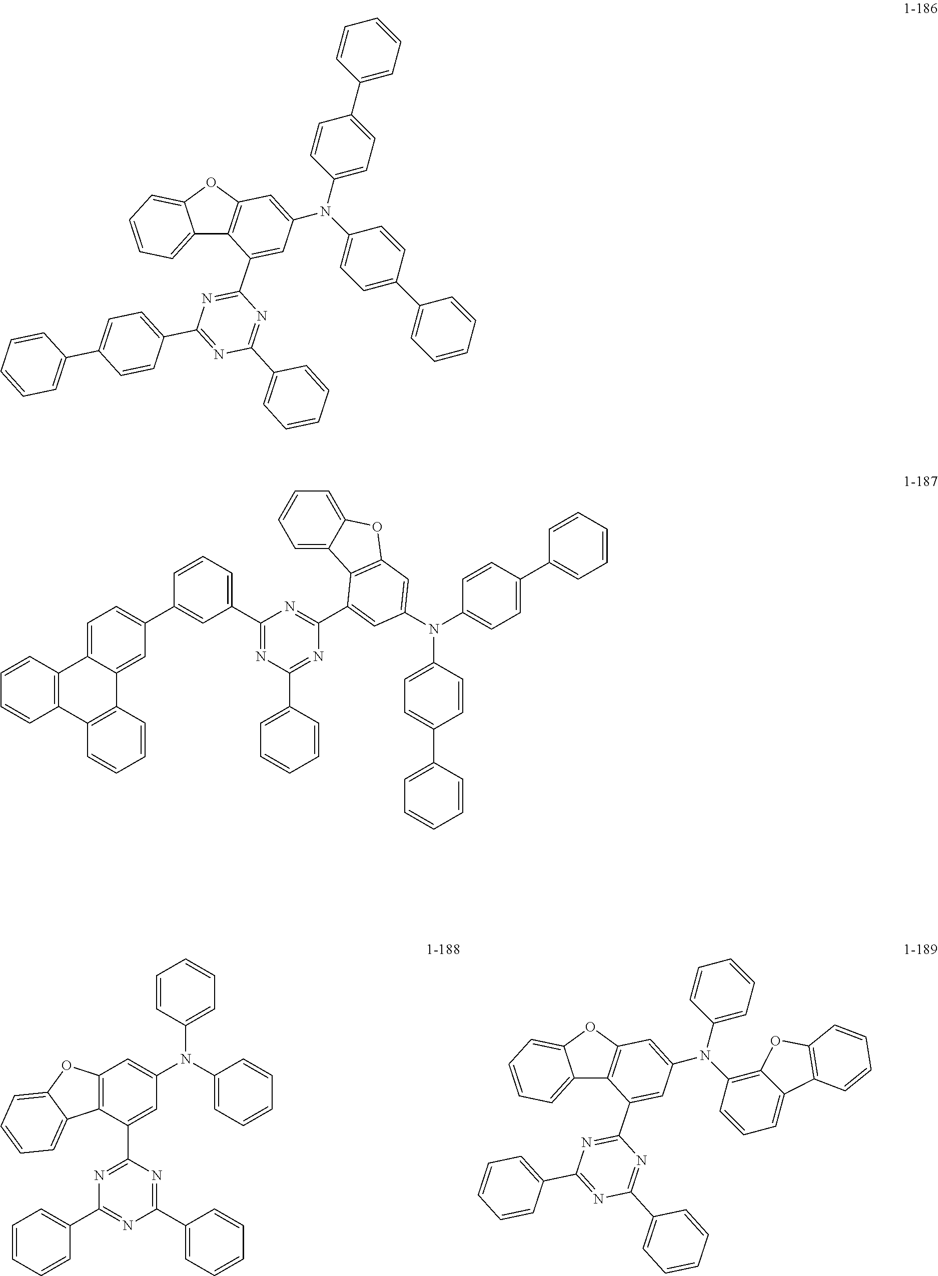 [SMILES: c1ccc(-c2ccc(-c3nc(-c4ccccc4)nc(-c4cc(N(c5ccc(-c6ccccc6)cc5)c5ccc(-c6ccccc6)cc5)cc5oc6ccccc6c45)n3)cc2)cc1 ; c1ccc(-c2ccc(N(c3ccc(-c4ccccc4)cc3)c3cc(-c4nc(-c5ccccc5)nc(-c5cccc(-c6ccc7c8ccccc8c8ccccc8c7c6)c5)n4)c4c(c3)oc3ccccc34)cc2)cc1 ; c1ccc(-c2nc(-c3ccccc3)nc(-c3cc(N(c4ccccc4)c4ccccc4)cc4oc5ccccc5c34)n2)cc1 ; c1ccc(-c2nc(-c3ccccc3)nc(-c3cc(N(c4ccccc4)c4cccc5c4oc4ccccc45)cc4oc5ccccc5c34)n2)cc1]

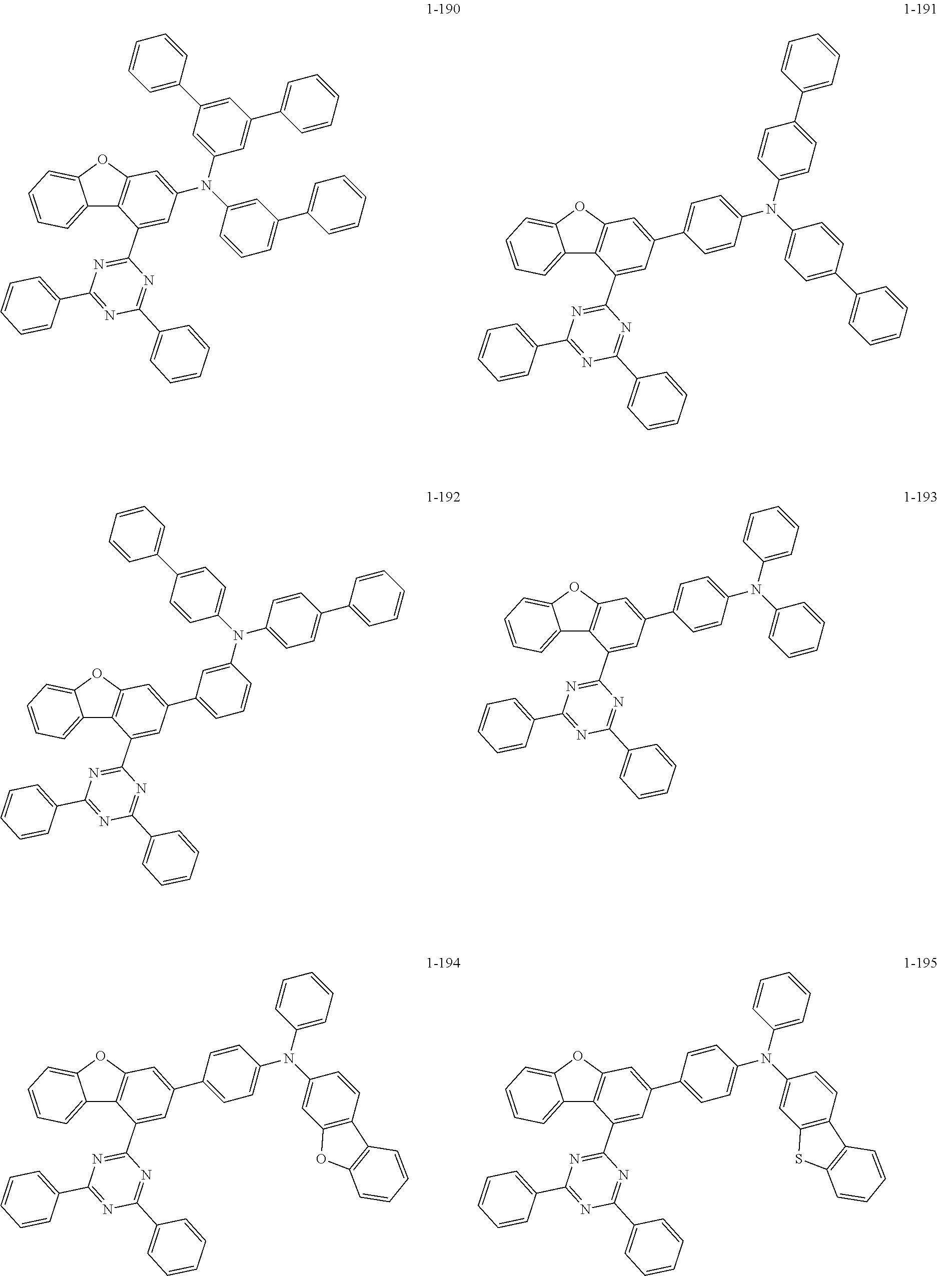

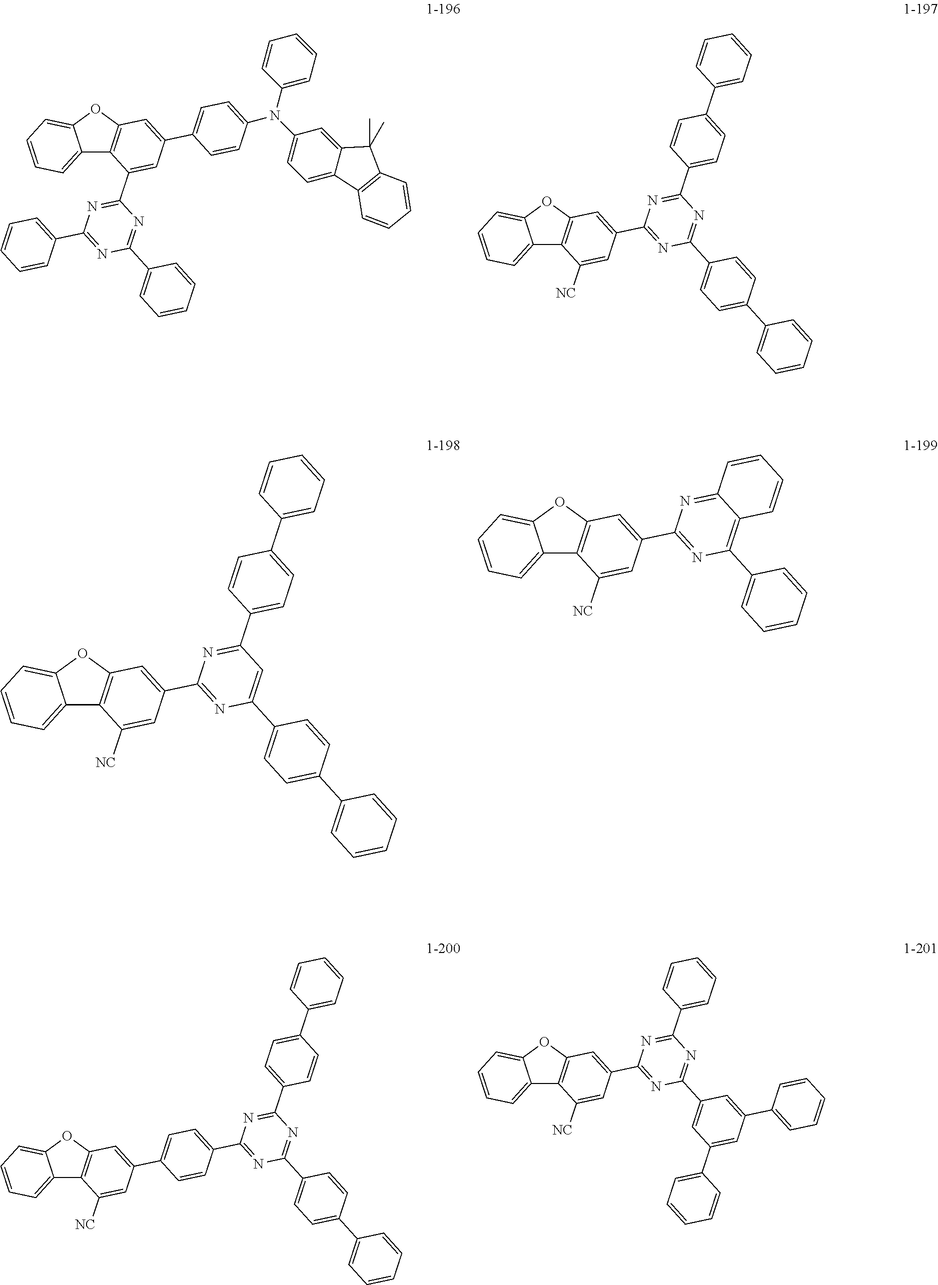 [SMILES: CC1(C)c2ccccc2-c2ccc(N(c3ccccc3)c3ccc(-c4cc(-c5nc(-c6ccccc6)nc(-c6ccccc6)n5)c5c(c4)oc4ccccc45)cc3)cc21 ; N#Cc1cc(-c2nc(-c3ccc(-c4ccccc4)cc3)nc(-c3ccc(-c4ccccc4)cc3)n2)cc2oc3ccccc3c12 ; N#Cc1cc(-c2nc(-c3ccc(-c4ccccc4)cc3)cc(-c3ccc(-c4ccccc4)cc3)n2)cc2oc3ccccc3c12 ; N#Cc1cc(-c2nc(-c3ccccc3)c3ccccc3n2)cc2oc3ccccc3c12 ; N#Cc1cc(-c2ccc(-c3nc(-c4ccc(-c5ccccc5)cc4)nc(-c4ccc(-c5ccccc5)cc4)n3)cc2)cc2oc3ccccc3c12 ; N#Cc1cc(-c2nc(-c3ccccc3)nc(-c3cc(-c4ccccc4)cc(-c4ccccc4)c3)n2)cc2oc3ccccc3c12]

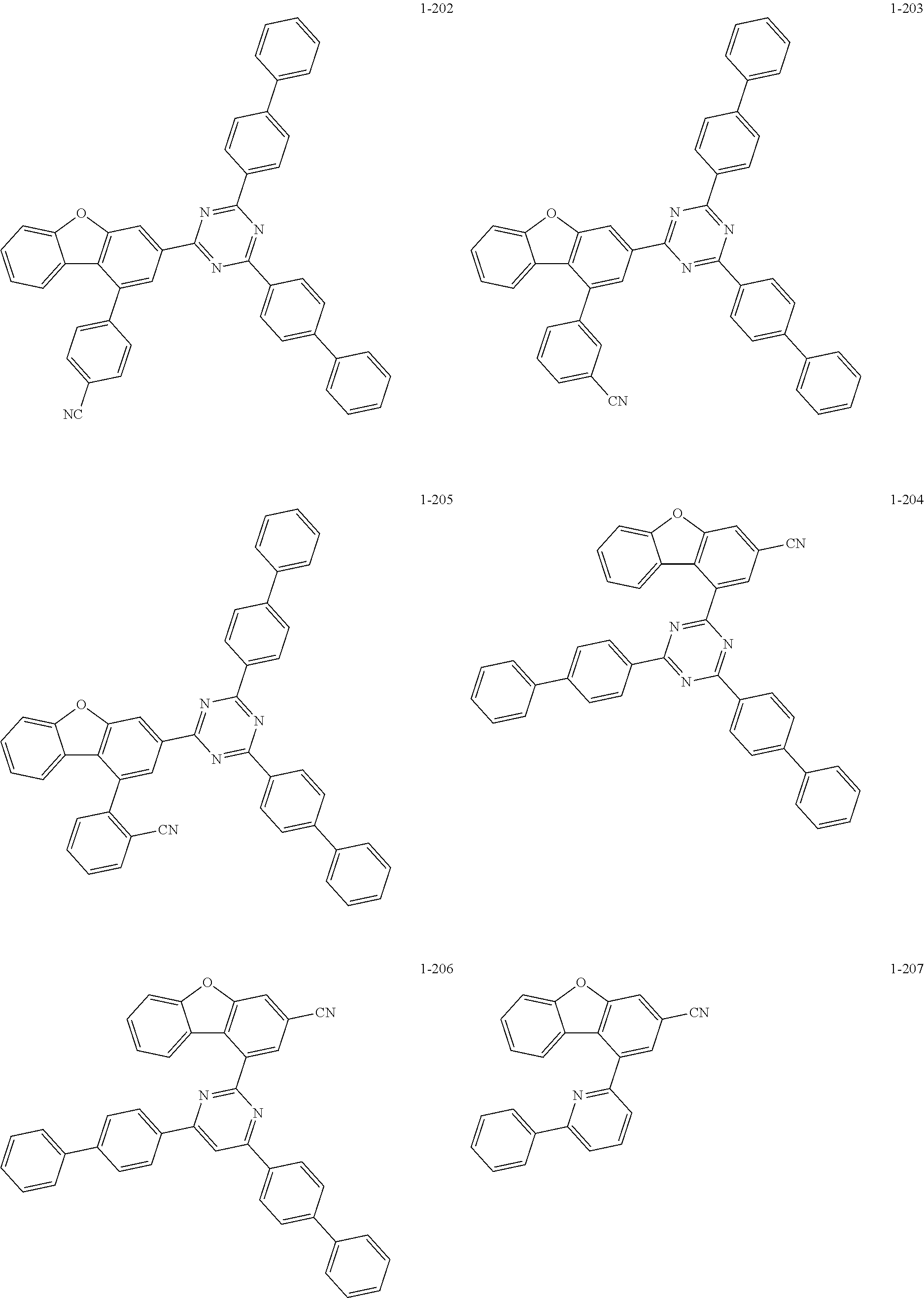 [SMILES: N#Cc1ccc(-c2cc(-c3nc(-c4ccc(-c5ccccc5)cc4)nc(-c4ccc(-c5ccccc5)cc4)n3)cc3oc4ccccc4c23)cc1 ; N#Cc1cccc(-c2cc(-c3nc(-c4ccc(-c5ccccc5)cc4)nc(-c4ccc(-c5ccccc5)cc4)n3)cc3oc4ccccc4c23)c1 ; N#Cc1ccccc1-c1cc(-c2nc(-c3ccc(-c4ccccc4)cc3)nc(-c3ccc(-c4ccccc4)cc3)n2)cc2oc3ccccc3c12 ; N#Cc1cc(-c2nc(-c3ccc(-c4ccccc4)cc3)nc(-c3ccc(-c4ccccc4)cc3)n2)c2c(c1)oc1ccccc12 ; N#Cc1cc(-c2nc(-c3ccc(-c4ccccc4)cc3)cc(-c3ccc(-c4ccccc4)cc3)n2)c2c(c1)oc1ccccc12 ; N#Cc1cc(-c2cccc(-c3ccccc3)n2)c2c(c1)oc1ccccc12]

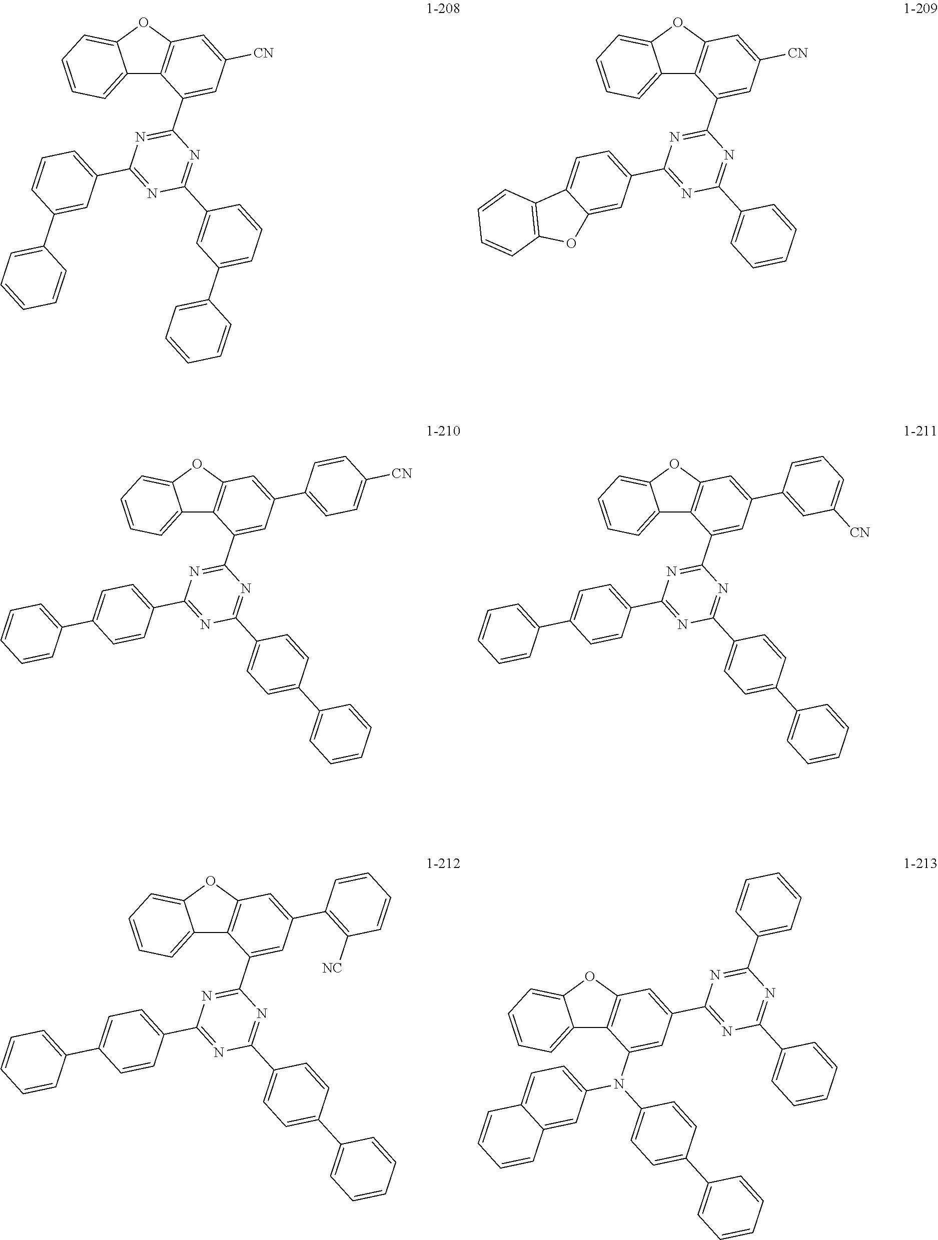

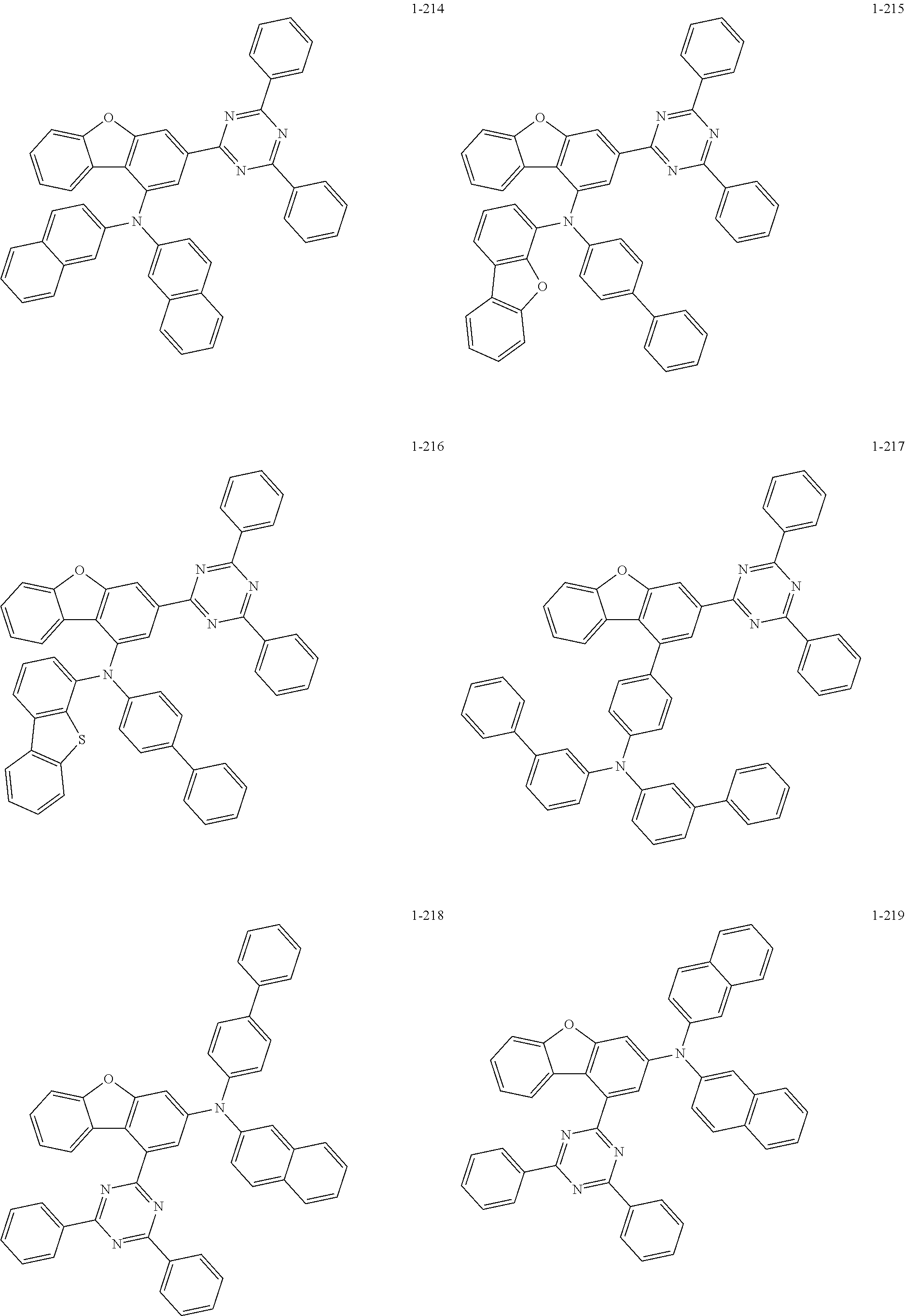

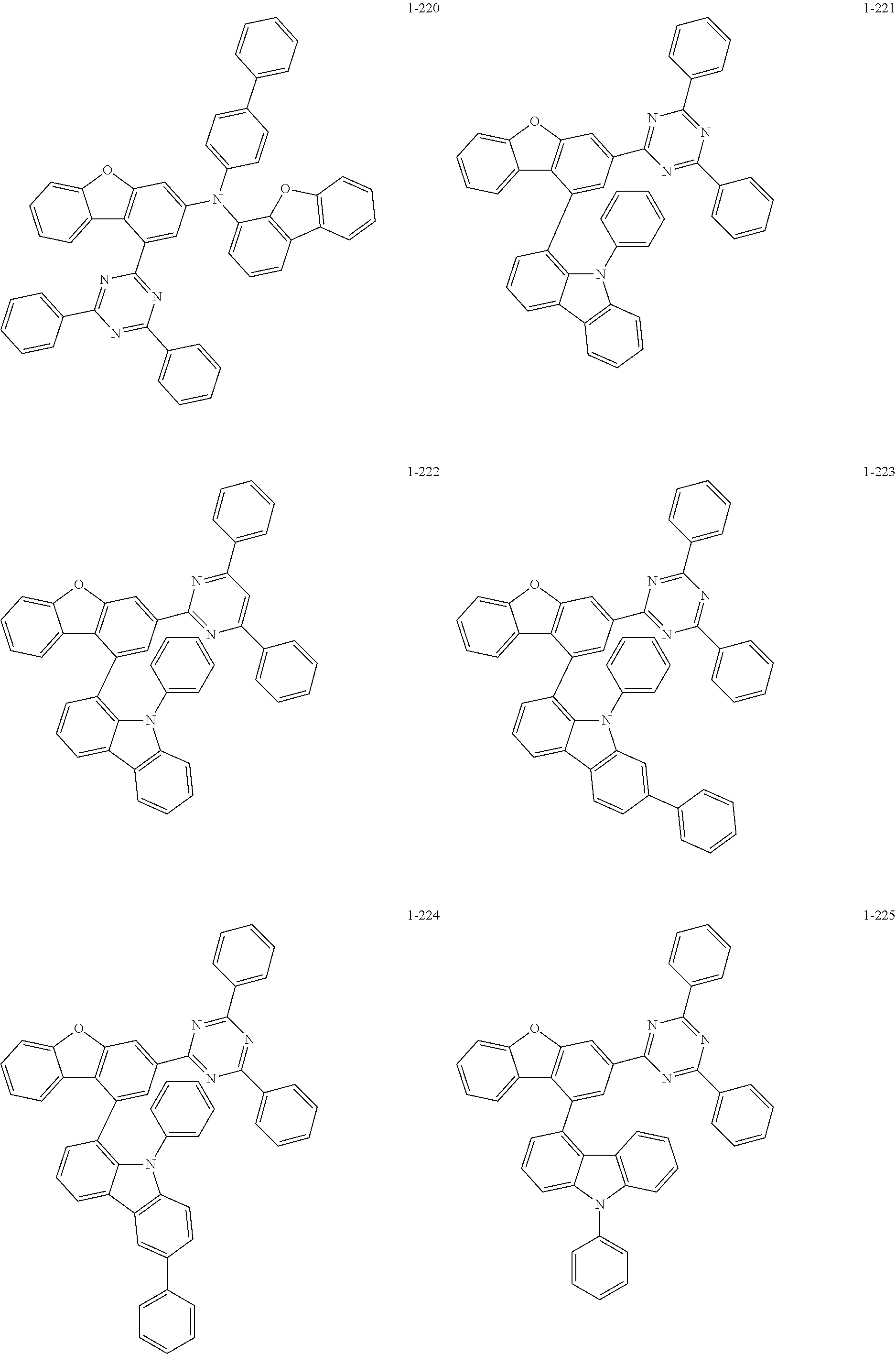

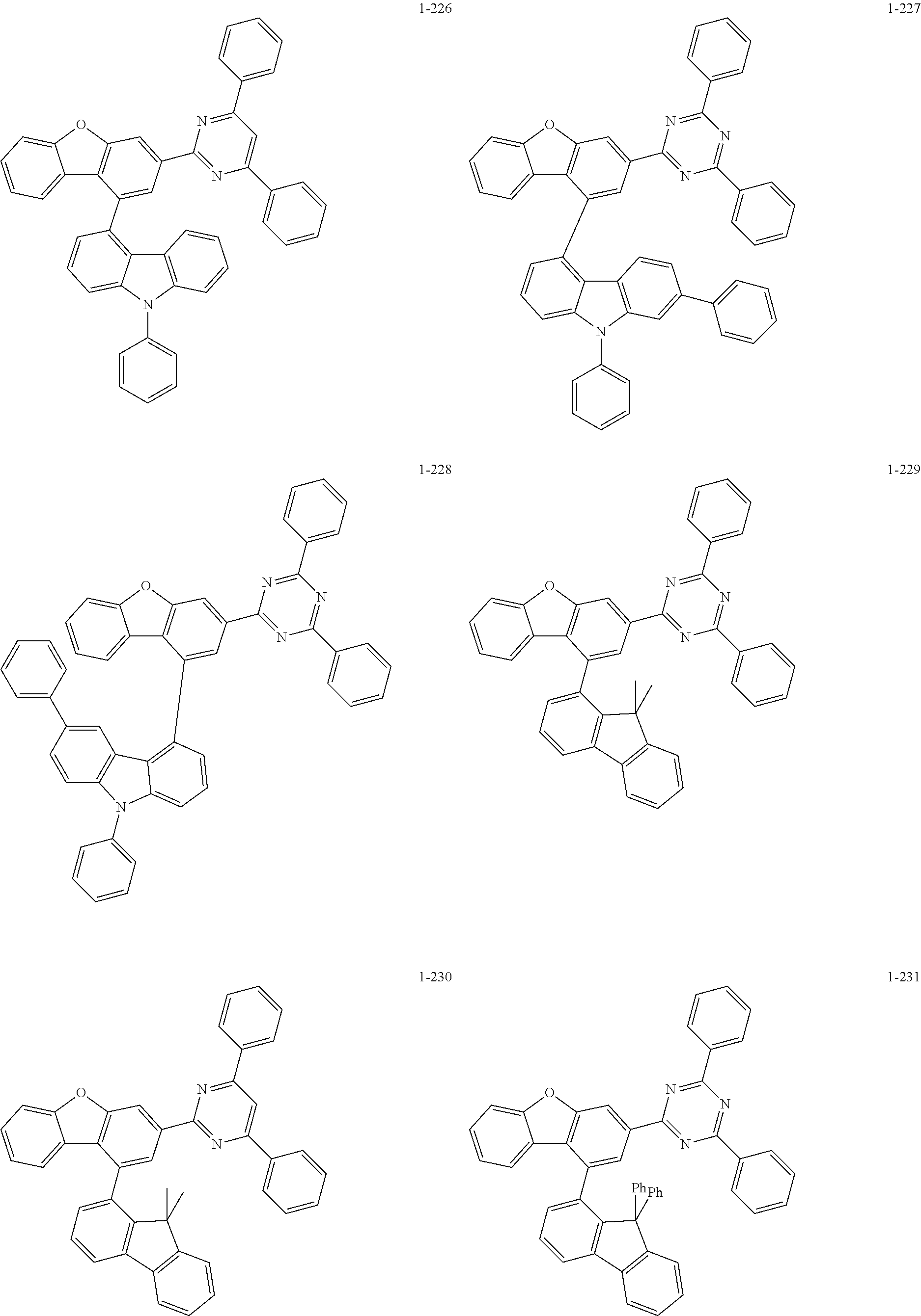

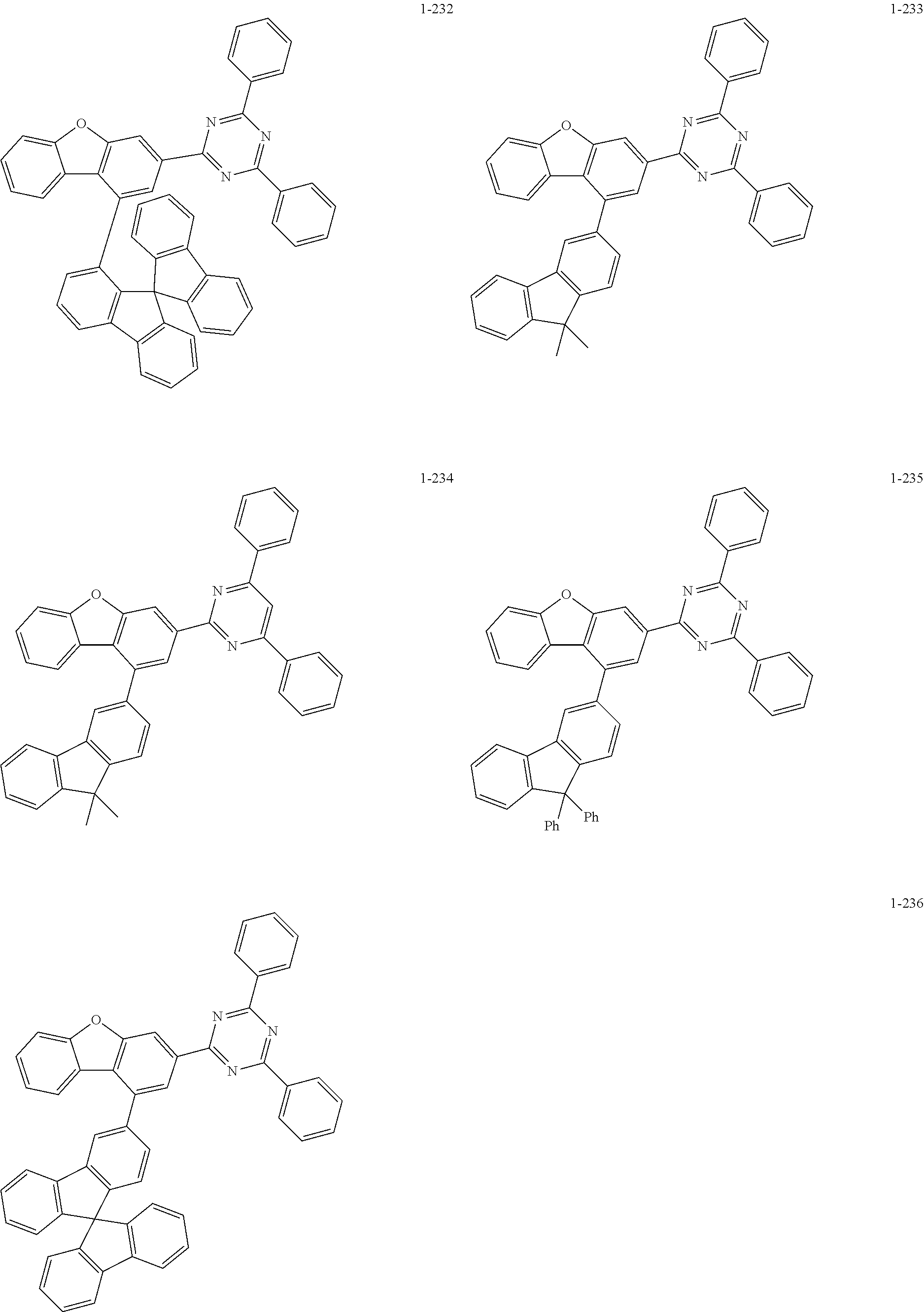 [SMILES: c1ccc(-c2nc(-c3ccccc3)nc(-c3cc(-c4cccc5c4C4(c6ccccc6-c6ccccc64)c4ccccc4-5)c4c(c3)oc3ccccc34)n2)cc1 ; CC1(C)c2ccccc2-c2cc(-c3cc(-c4nc(-c5ccccc5)nc(-c5ccccc5)n4)cc4oc5ccccc5c34)ccc21 ; CC1(C)c2ccccc2-c2cc(-c3cc(-c4nc(-c5ccccc5)cc(-c5ccccc5)n4)cc4oc5ccccc5c34)ccc21 ; c1ccc(-c2nc(-c3ccccc3)nc(-c3cc(-c4ccc5c(c4)-c4ccccc4C5(c4ccccc4)c4ccccc4)c4c(c3)oc3ccccc34)n2)cc1 ; c1ccc(-c2nc(-c3ccccc3)nc(-c3cc(-c4ccc5c(c4)-c4ccccc4C54c5ccccc5-c5ccccc54)c4c(c3)oc3ccccc34)n2)cc1]

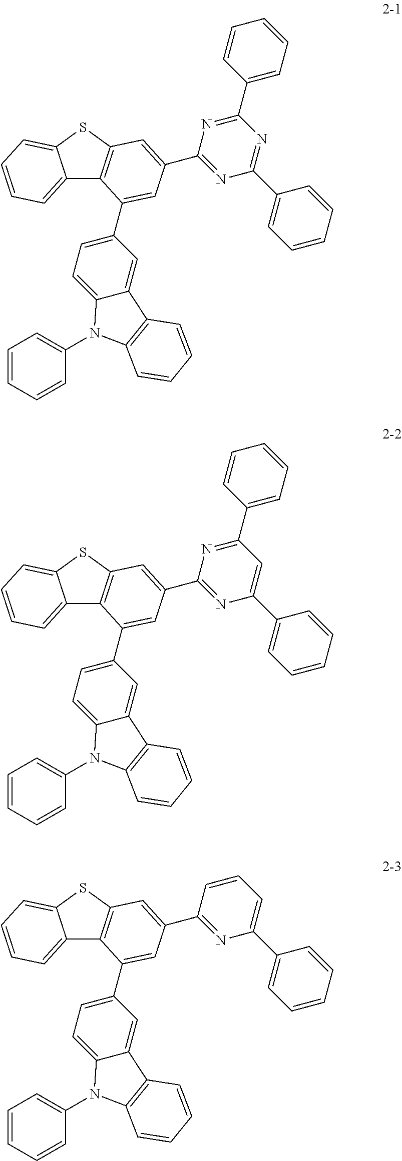 [SMILES: c1ccc(-c2nc(-c3ccccc3)nc(-c3cc(-c4ccc5c(c4)c4ccccc4n5-c4ccccc4)c4c(c3)sc3ccccc34)n2)cc1 ; c1ccc(-c2cc(-c3ccccc3)nc(-c3cc(-c4ccc5c(c4)c4ccccc4n5-c4ccccc4)c4c(c3)sc3ccccc34)n2)cc1 ; c1ccc(-c2cccc(-c3cc(-c4ccc5c(c4)c4ccccc4n5-c4ccccc4)c4c(c3)sc3ccccc34)n2)cc1]

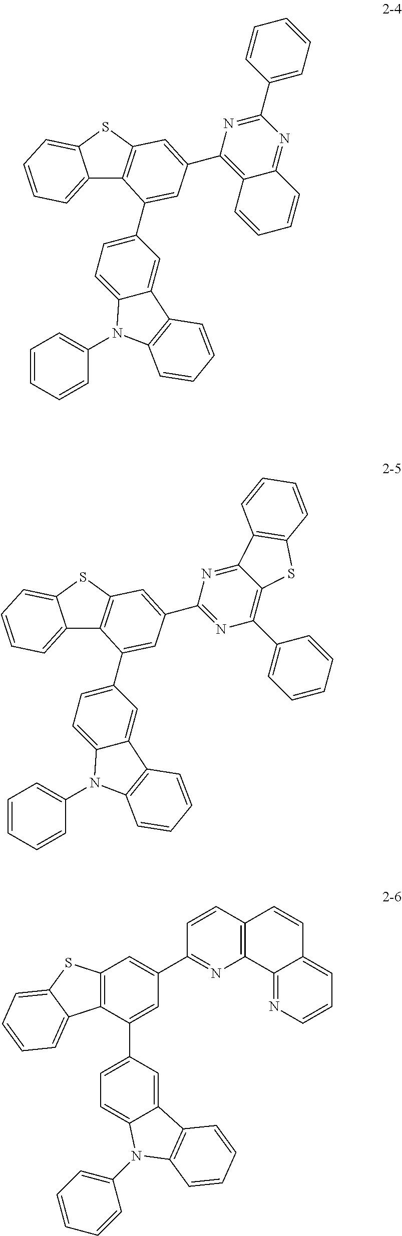 [SMILES: c1ccc(-c2nc(-c3cc(-c4ccc5c(c4)c4ccccc4n5-c4ccccc4)c4c(c3)sc3ccccc34)c3ccccc3n2)cc1 ; c1ccc(-c2nc(-c3cc(-c4ccc5c(c4)c4ccccc4n5-c4ccccc4)c4c(c3)sc3ccccc34)nc3c2sc2ccccc23)cc1 ; c1ccc(-n2c3ccccc3c3cc(-c4cc(-c5ccc6ccc7cccnc7c6n5)cc5sc6ccccc6c45)ccc32)cc1]

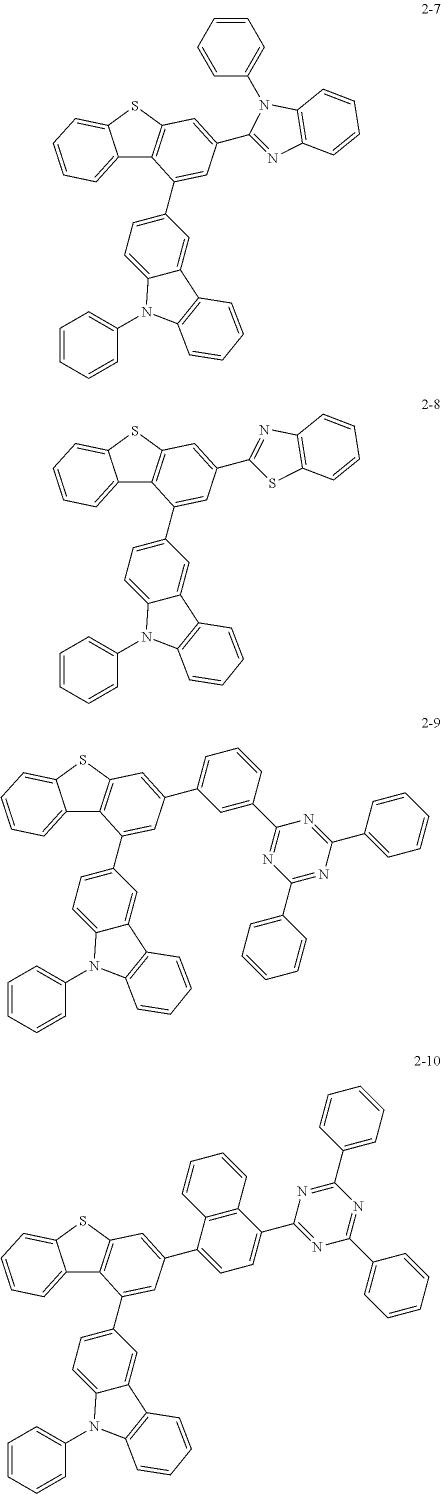 [SMILES: c1ccc(-n2c(-c3cc(-c4ccc5c(c4)c4ccccc4n5-c4ccccc4)c4c(c3)sc3ccccc34)nc3ccccc32)cc1 ; c1ccc(-n2c3ccccc3c3cc(-c4cc(-c5nc6ccccc6s5)cc5sc6ccccc6c45)ccc32)cc1 ; c1ccc(-c2nc(-c3ccccc3)nc(-c3cccc(-c4cc(-c5ccc6c(c5)c5ccccc5n6-c5ccccc5)c5c(c4)sc4ccccc45)c3)n2)cc1 ; c1ccc(-c2nc(-c3ccccc3)nc(-c3ccc(-c4cc(-c5ccc6c(c5)c5ccccc5n6-c5ccccc5)c5c(c4)sc4ccccc45)c4ccccc34)n2)cc1]

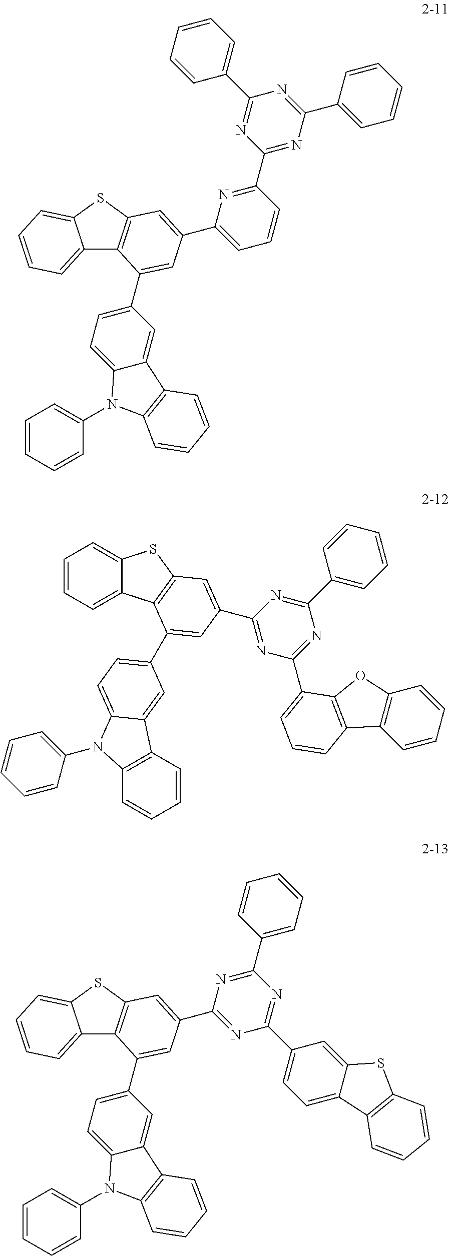 [SMILES: c1ccc(-c2nc(-c3ccccc3)nc(-c3cccc(-c4cc(-c5ccc6c(c5)c5ccccc5n6-c5ccccc5)c5c(c4)sc4ccccc45)n3)n2)cc1 ; c1ccc(-c2nc(-c3cc(-c4ccc5c(c4)c4ccccc4n5-c4ccccc4)c4c(c3)sc3ccccc34)nc(-c3cccc4c3oc3ccccc34)n2)cc1 ; c1ccc(-c2nc(-c3ccc4c(c3)sc3ccccc34)nc(-c3cc(-c4ccc5c(c4)c4ccccc4n5-c4ccccc4)c4c(c3)sc3ccccc34)n2)cc1]

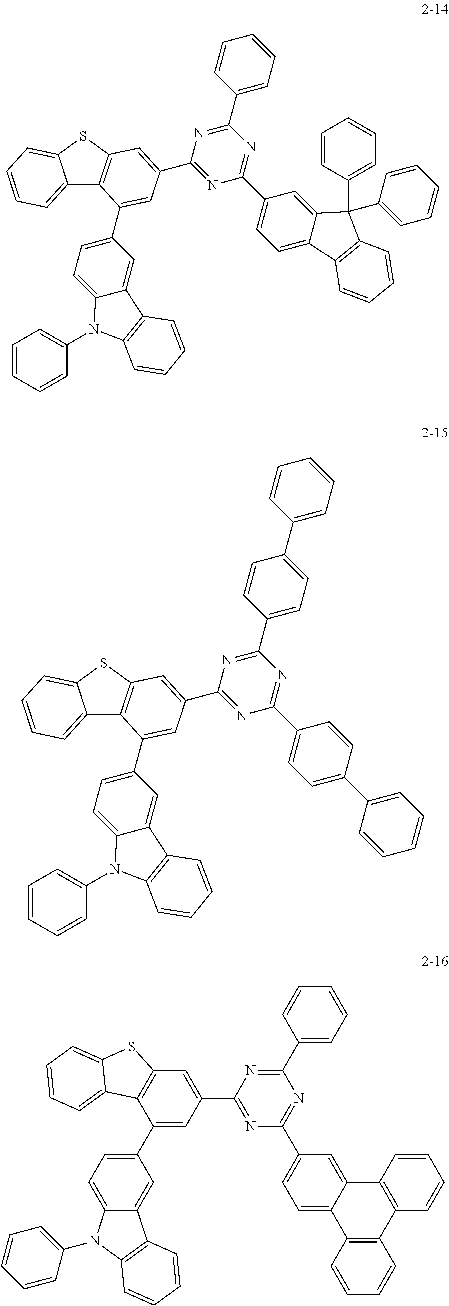 [SMILES: c1ccc(-c2nc(-c3ccc4c(c3)C(c3ccccc3)(c3ccccc3)c3ccccc3-4)nc(-c3cc(-c4ccc5c(c4)c4ccccc4n5-c4ccccc4)c4c(c3)sc3ccccc34)n2)cc1 ; c1ccc(-c2ccc(-c3nc(-c4ccc(-c5ccccc5)cc4)nc(-c4cc(-c5ccc6c(c5)c5ccccc5n6-c5ccccc5)c5c(c4)sc4ccccc45)n3)cc2)cc1 ; c1ccc(-c2nc(-c3cc(-c4ccc5c(c4)c4ccccc4n5-c4ccccc4)c4c(c3)sc3ccccc34)nc(-c3ccc4c5ccccc5c5ccccc5c4c3)n2)cc1]

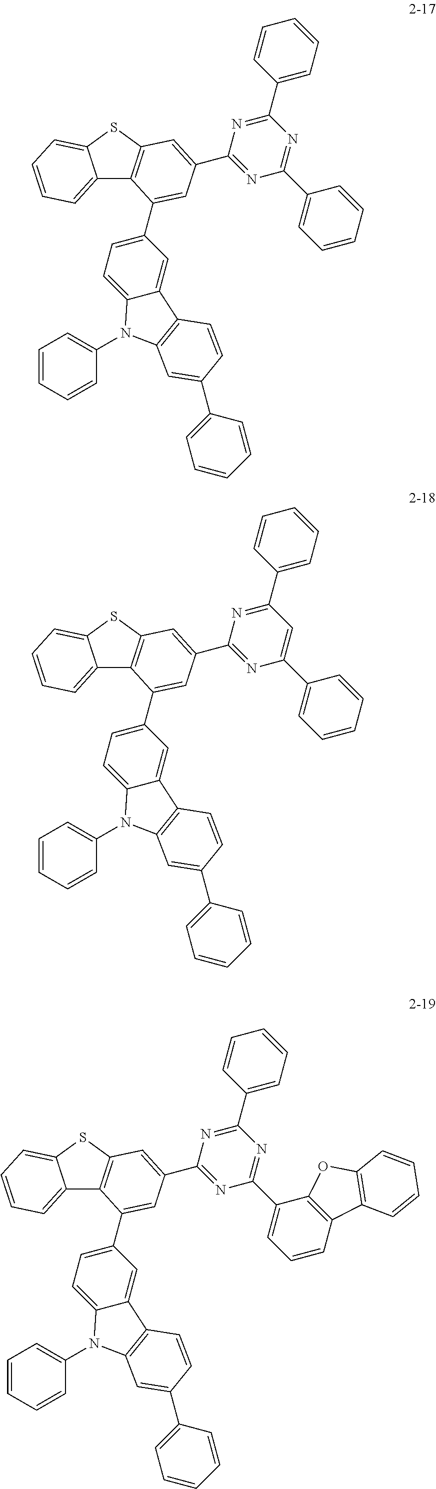 [SMILES: c1ccc(-c2ccc3c4cc(-c5cc(-c6nc(-c7ccccc7)nc(-c7ccccc7)n6)cc6sc7ccccc7c56)ccc4n(-c4ccccc4)c3c2)cc1 ; c1ccc(-c2ccc3c4cc(-c5cc(-c6nc(-c7ccccc7)cc(-c7ccccc7)n6)cc6sc7ccccc7c56)ccc4n(-c4ccccc4)c3c2)cc1 ; c1ccc(-c2ccc3c4cc(-c5cc(-c6nc(-c7ccccc7)nc(-c7cccc8c7oc7ccccc78)n6)cc6sc7ccccc7c56)ccc4n(-c4ccccc4)c3c2)cc1]

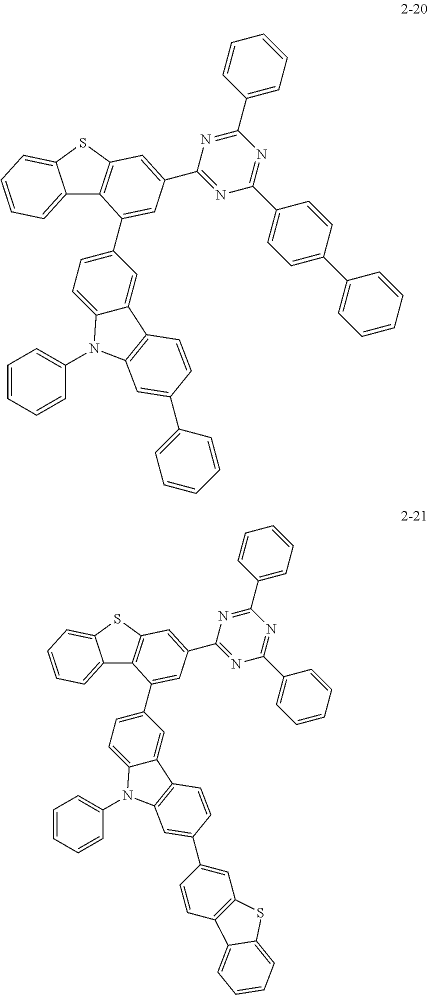 [SMILES: c1ccc(-c2ccc(-c3nc(-c4ccccc4)nc(-c4cc(-c5ccc6c(c5)c5ccc(-c7ccccc7)cc5n6-c5ccccc5)c5c(c4)sc4ccccc45)n3)cc2)cc1 ; c1ccc(-c2nc(-c3ccccc3)nc(-c3cc(-c4ccc5c(c4)c4ccc(-c6ccc7c(c6)sc6ccccc67)cc4n5-c4ccccc4)c4c(c3)sc3ccccc34)n2)cc1]

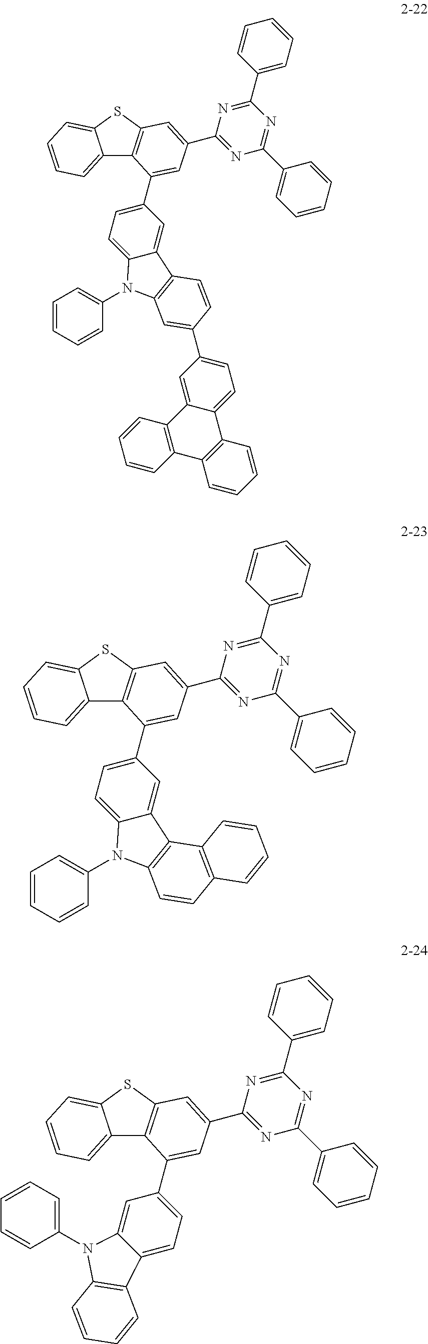 [SMILES: c1ccc(-c2nc(-c3ccccc3)nc(-c3cc(-c4ccc5c(c4)c4ccc(-c6ccc7c8ccccc8c8ccccc8c7c6)cc4n5-c4ccccc4)c4c(c3)sc3ccccc34)n2)cc1 ; c1ccc(-c2nc(-c3ccccc3)nc(-c3cc(-c4ccc5c(c4)c4c6ccccc6ccc4n5-c4ccccc4)c4c(c3)sc3ccccc34)n2)cc1 ; c1ccc(-c2nc(-c3ccccc3)nc(-c3cc(-c4ccc5c6ccccc6n(-c6ccccc6)c5c4)c4c(c3)sc3ccccc34)n2)cc1]

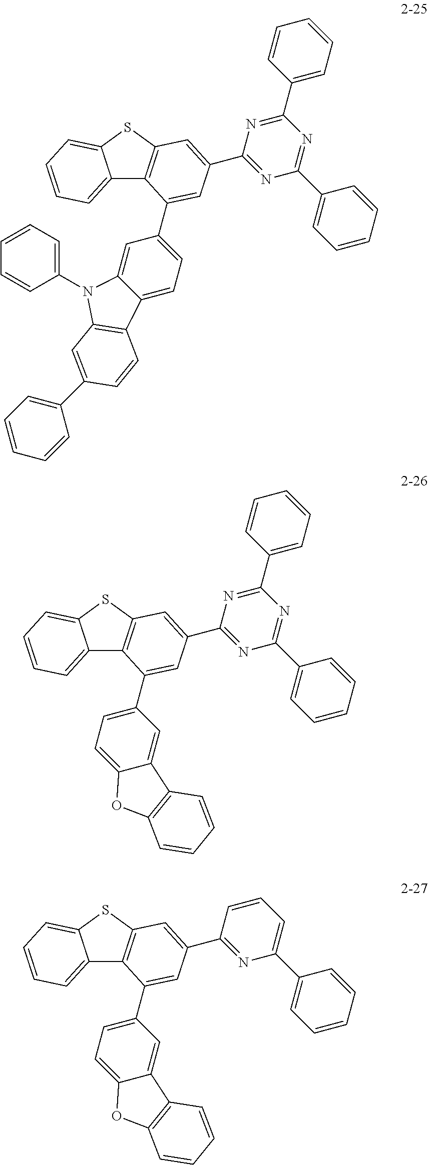 [SMILES: c1ccc(-c2ccc3c4ccc(-c5cc(-c6nc(-c7ccccc7)nc(-c7ccccc7)n6)cc6sc7ccccc7c56)cc4n(-c4ccccc4)c3c2)cc1 ; c1ccc(-c2nc(-c3ccccc3)nc(-c3cc(-c4ccc5oc6ccccc6c5c4)c4c(c3)sc3ccccc34)n2)cc1 ; c1ccc(-c2cccc(-c3cc(-c4ccc5oc6ccccc6c5c4)c4c(c3)sc3ccccc34)n2)cc1]

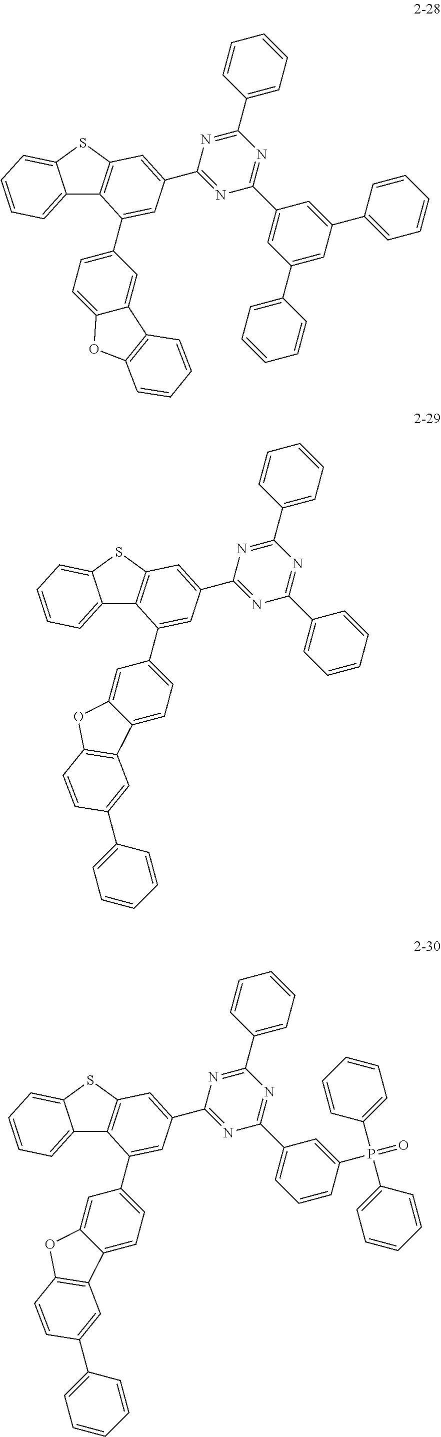 [SMILES: c1ccc(-c2cc(-c3ccccc3)cc(-c3nc(-c4ccccc4)nc(-c4cc(-c5ccc6oc7ccccc7c6c5)c5c(c4)sc4ccccc45)n3)c2)cc1 ; c1ccc(-c2ccc3oc4cc(-c5cc(-c6nc(-c7ccccc7)nc(-c7ccccc7)n6)cc6sc7ccccc7c56)ccc4c3c2)cc1 ; O=P(c1ccccc1)(c1ccccc1)c1cccc(-c2nc(-c3ccccc3)nc(-c3cc(-c4ccc5c(c4)oc4ccc(-c6ccccc6)cc45)c4c(c3)sc3ccccc34)n2)c1]

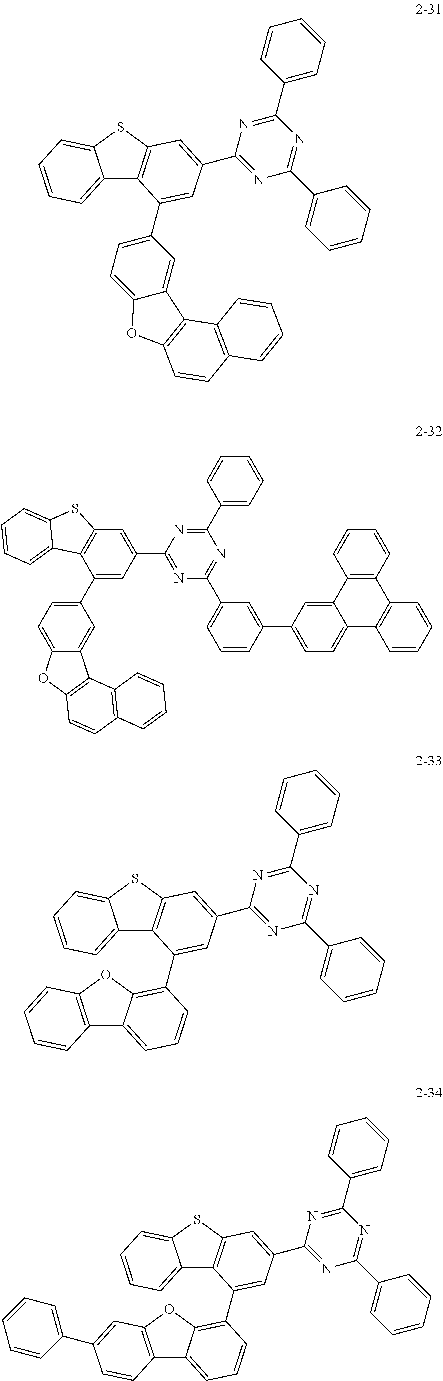 [SMILES: c1ccc(-c2nc(-c3ccccc3)nc(-c3cc(-c4ccc5oc6ccc7ccccc7c6c5c4)c4c(c3)sc3ccccc34)n2)cc1 ; c1ccc(-c2nc(-c3cccc(-c4ccc5c6ccccc6c6ccccc6c5c4)c3)nc(-c3cc(-c4ccc5oc6ccc7ccccc7c6c5c4)c4c(c3)sc3ccccc34)n2)cc1 ; c1ccc(-c2nc(-c3ccccc3)nc(-c3cc(-c4cccc5c4oc4ccccc45)c4c(c3)sc3ccccc34)n2)cc1 ; c1ccc(-c2ccc3c(c2)oc2c(-c4cc(-c5nc(-c6ccccc6)nc(-c6ccccc6)n5)cc5sc6ccccc6c45)cccc23)cc1]

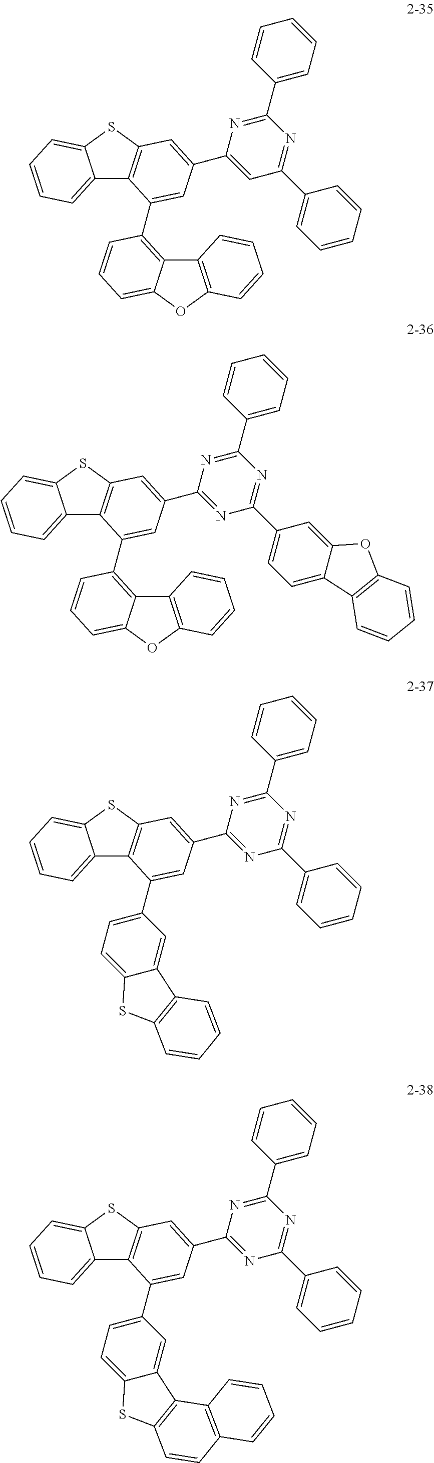 [SMILES: c1ccc(-c2cc(-c3cc(-c4cccc5oc6ccccc6c45)c4c(c3)sc3ccccc34)nc(-c3ccccc3)n2)cc1 ; c1ccc(-c2nc(-c3ccc4c(c3)oc3ccccc34)nc(-c3cc(-c4cccc5oc6ccccc6c45)c4c(c3)sc3ccccc34)n2)cc1 ; c1ccc(-c2nc(-c3ccccc3)nc(-c3cc(-c4ccc5sc6ccccc6c5c4)c4c(c3)sc3ccccc34)n2)cc1 ; c1ccc(-c2nc(-c3ccccc3)nc(-c3cc(-c4ccc5sc6ccc7ccccc7c6c5c4)c4c(c3)sc3ccccc34)n2)cc1]

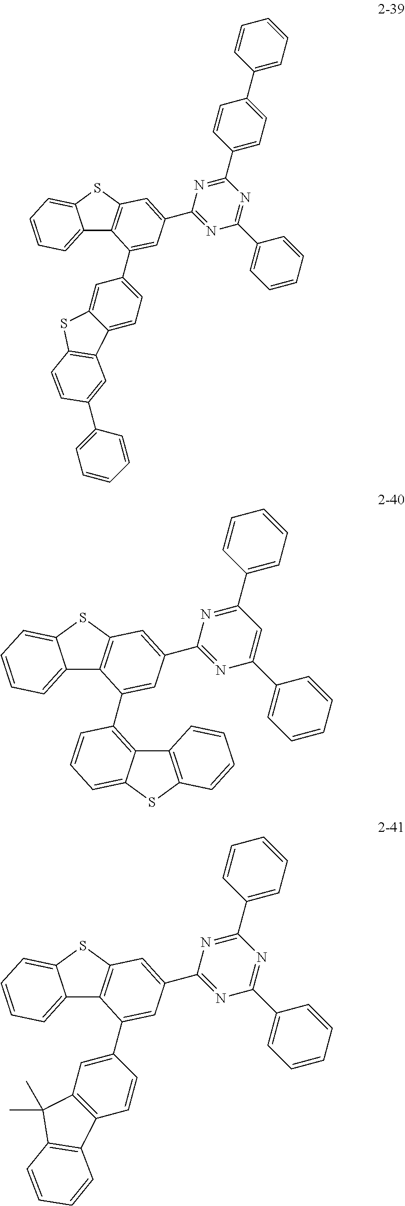 [SMILES: c1ccc(-c2ccc(-c3nc(-c4ccccc4)nc(-c4cc(-c5ccc6c(c5)sc5ccc(-c7ccccc7)cc56)c5c(c4)sc4ccccc45)n3)cc2)cc1 ; c1ccc(-c2cc(-c3ccccc3)nc(-c3cc(-c4cccc5sc6ccccc6c45)c4c(c3)sc3ccccc34)n2)cc1 ; CC1(C)c2ccccc2-c2ccc(-c3cc(-c4nc(-c5ccccc5)nc(-c5ccccc5)n4)cc4sc5ccccc5c34)cc21]

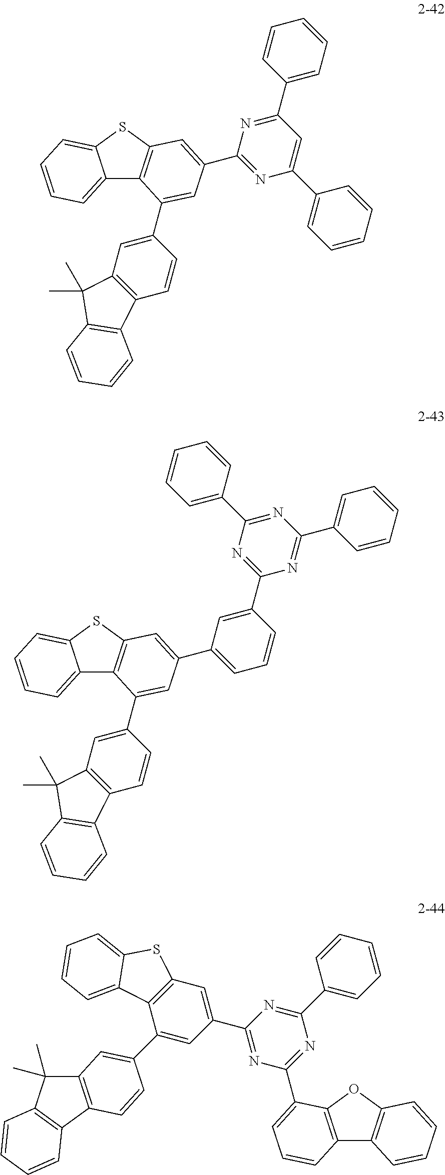 [SMILES: CC1(C)c2ccccc2-c2ccc(-c3cc(-c4nc(-c5ccccc5)cc(-c5ccccc5)n4)cc4sc5ccccc5c34)cc21 ; CC1(C)c2ccccc2-c2ccc(-c3cc(-c4cccc(-c5nc(-c6ccccc6)nc(-c6ccccc6)n5)c4)cc4sc5ccccc5c34)cc21 ; CC1(C)c2ccccc2-c2ccc(-c3cc(-c4nc(-c5ccccc5)nc(-c5cccc6c5oc5ccccc56)n4)cc4sc5ccccc5c34)cc21]

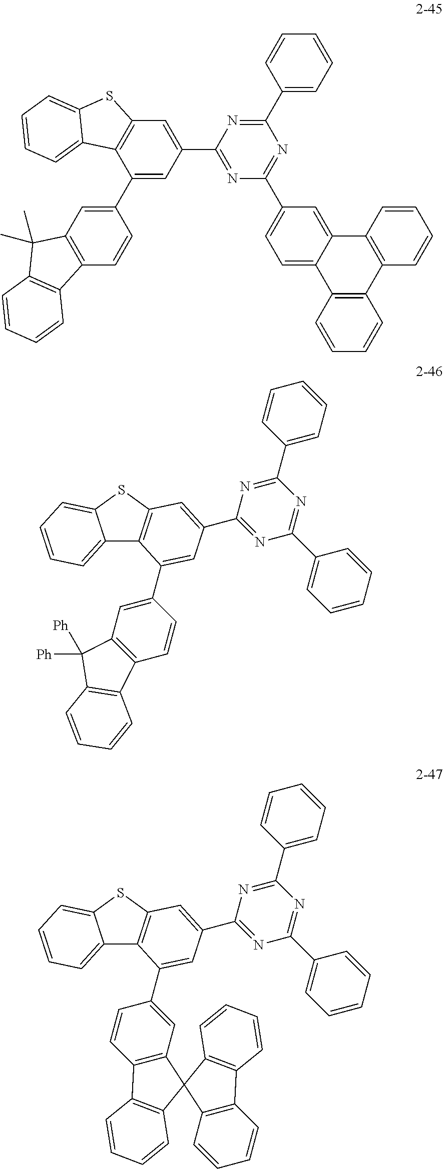

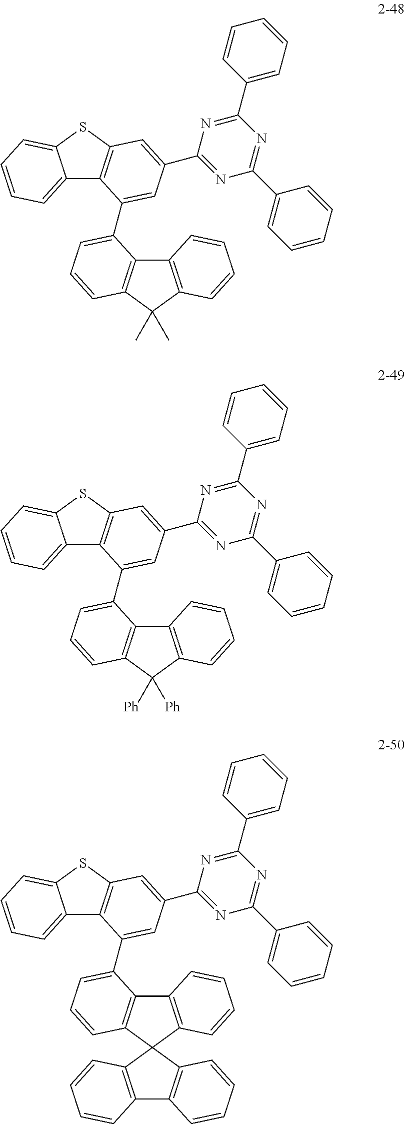 [SMILES: CC1(C)c2ccccc2-c2c(-c3cc(-c4nc(-c5ccccc5)nc(-c5ccccc5)n4)cc4sc5ccccc5c34)cccc21 ; c1ccc(-c2nc(-c3ccccc3)nc(-c3cc(-c4cccc5c4-c4ccccc4C5(c4ccccc4)c4ccccc4)c4c(c3)sc3ccccc34)n2)cc1 ; c1ccc(-c2nc(-c3ccccc3)nc(-c3cc(-c4cccc5c4-c4ccccc4C54c5ccccc5-c5ccccc54)c4c(c3)sc3ccccc34)n2)cc1]

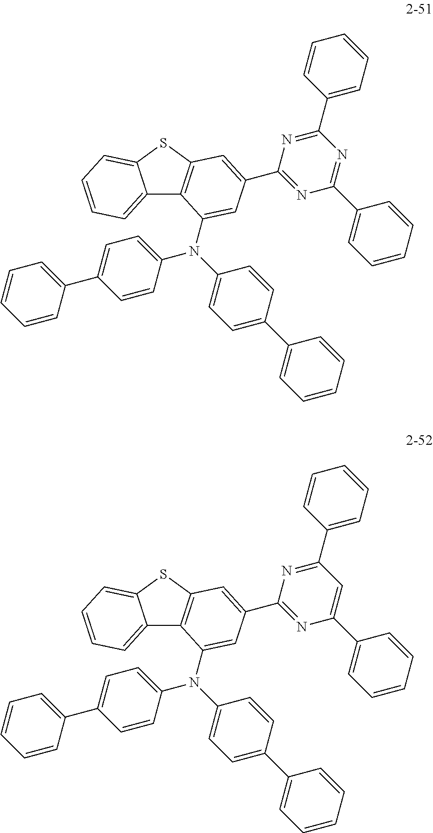 [SMILES: c1ccc(-c2ccc(N(c3ccc(-c4ccccc4)cc3)c3cc(-c4nc(-c5ccccc5)nc(-c5ccccc5)n4)cc4sc5ccccc5c34)cc2)cc1 ; c1ccc(-c2ccc(N(c3ccc(-c4ccccc4)cc3)c3cc(-c4nc(-c5ccccc5)cc(-c5ccccc5)n4)cc4sc5ccccc5c34)cc2)cc1]

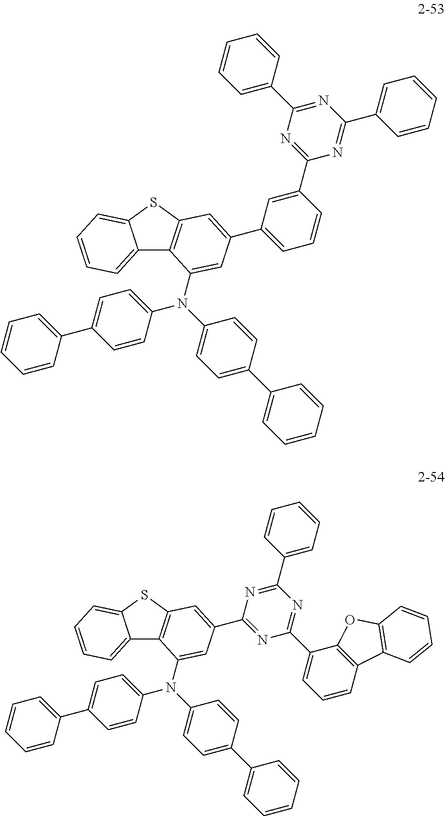 [SMILES: c1ccc(-c2ccc(N(c3ccc(-c4ccccc4)cc3)c3cc(-c4cccc(-c5nc(-c6ccccc6)nc(-c6ccccc6)n5)c4)cc4sc5ccccc5c34)cc2)cc1 ; c1ccc(-c2ccc(N(c3ccc(-c4ccccc4)cc3)c3cc(-c4nc(-c5ccccc5)nc(-c5cccc6c5oc5ccccc56)n4)cc4sc5ccccc5c34)cc2)cc1]

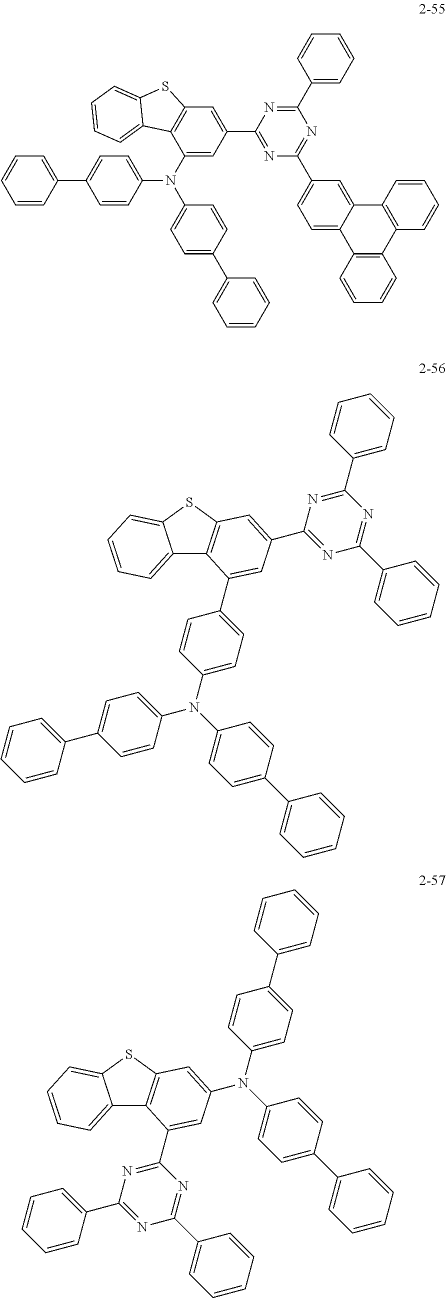

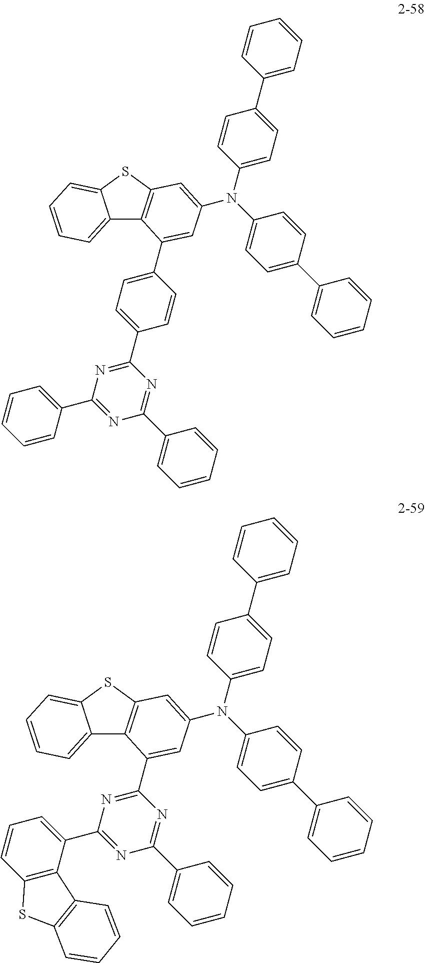 [SMILES: c1ccc(-c2ccc(N(c3ccc(-c4ccccc4)cc3)c3cc(-c4ccc(-c5nc(-c6ccccc6)nc(-c6ccccc6)n5)cc4)c4c(c3)sc3ccccc34)cc2)cc1 ; c1ccc(-c2ccc(N(c3ccc(-c4ccccc4)cc3)c3cc(-c4nc(-c5ccccc5)nc(-c5cccc6sc7ccccc7c56)n4)c4c(c3)sc3ccccc34)cc2)cc1]

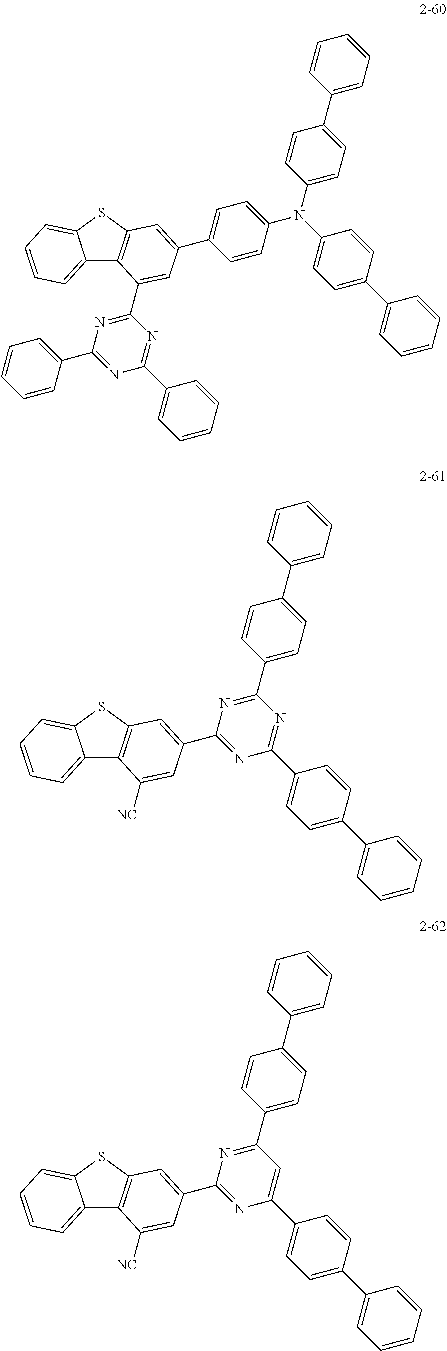 [SMILES: c1ccc(-c2ccc(N(c3ccc(-c4ccccc4)cc3)c3ccc(-c4cc(-c5nc(-c6ccccc6)nc(-c6ccccc6)n5)c5c(c4)sc4ccccc45)cc3)cc2)cc1 ; N#Cc1cc(-c2nc(-c3ccc(-c4ccccc4)cc3)nc(-c3ccc(-c4ccccc4)cc3)n2)cc2sc3ccccc3c12 ; N#Cc1cc(-c2nc(-c3ccc(-c4ccccc4)cc3)cc(-c3ccc(-c4ccccc4)cc3)n2)cc2sc3ccccc3c12]

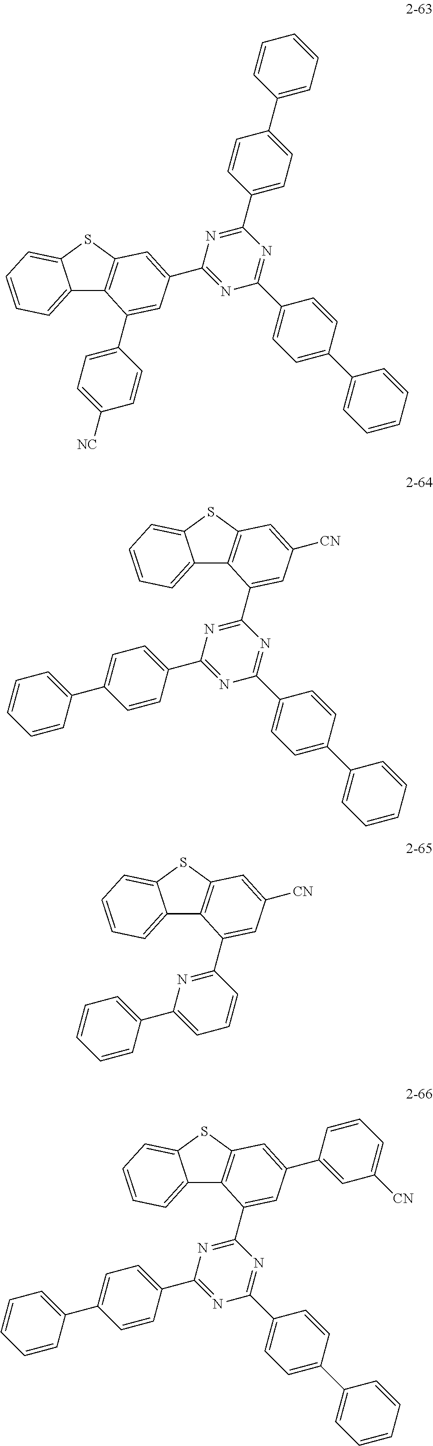 [SMILES: N#Cc1ccc(-c2cc(-c3nc(-c4ccc(-c5ccccc5)cc4)nc(-c4ccc(-c5ccccc5)cc4)n3)cc3sc4ccccc4c23)cc1 ; N#Cc1cc(-c2nc(-c3ccc(-c4ccccc4)cc3)nc(-c3ccc(-c4ccccc4)cc3)n2)c2c(c1)sc1ccccc12 ; N#Cc1cc(-c2cccc(-c3ccccc3)n2)c2c(c1)sc1ccccc12 ; N#Cc1cccc(-c2cc(-c3nc(-c4ccc(-c5ccccc5)cc4)nc(-c4ccc(-c5ccccc5)cc4)n3)c3c(c2)sc2ccccc23)c1]

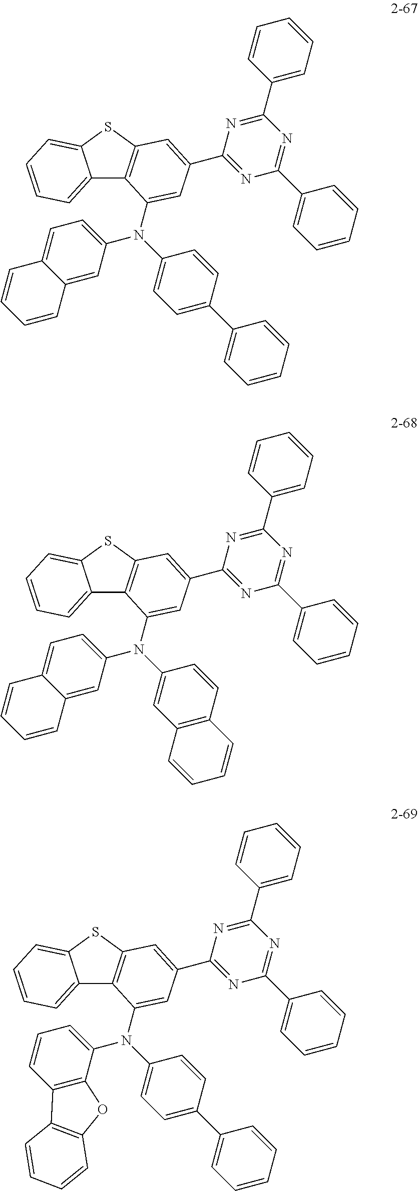 [SMILES: c1ccc(-c2ccc(N(c3ccc4ccccc4c3)c3cc(-c4nc(-c5ccccc5)nc(-c5ccccc5)n4)cc4sc5ccccc5c34)cc2)cc1 ; c1ccc(-c2nc(-c3ccccc3)nc(-c3cc(N(c4ccc5ccccc5c4)c4ccc5ccccc5c4)c4c(c3)sc3ccccc34)n2)cc1 ; c1ccc(-c2ccc(N(c3cccc4c3oc3ccccc34)c3cc(-c4nc(-c5ccccc5)nc(-c5ccccc5)n4)cc4sc5ccccc5c34)cc2)cc1]

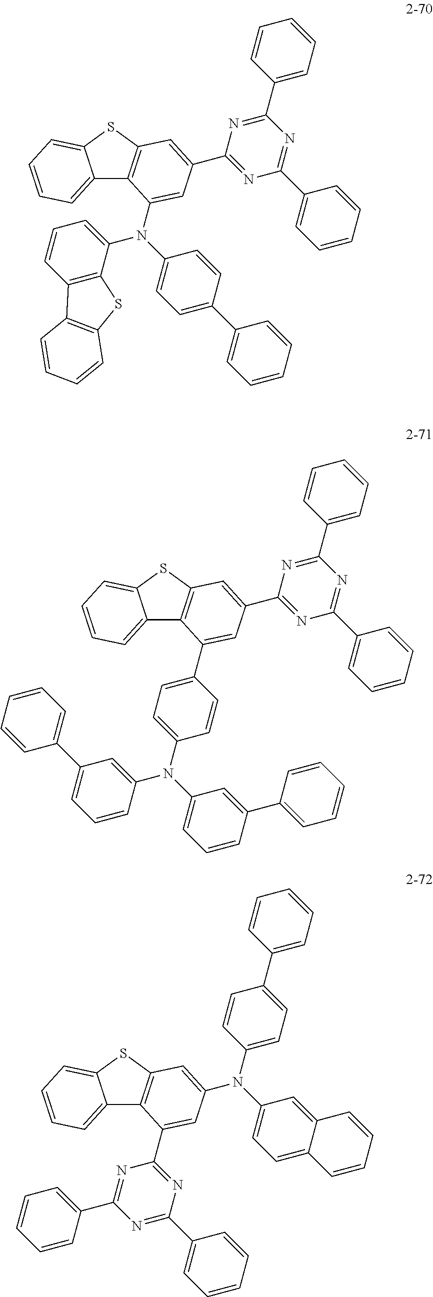 [SMILES: c1ccc(-c2ccc(N(c3cccc4c3sc3ccccc34)c3cc(-c4nc(-c5ccccc5)nc(-c5ccccc5)n4)cc4sc5ccccc5c34)cc2)cc1 ; c1ccc(-c2cccc(N(c3ccc(-c4cc(-c5nc(-c6ccccc6)nc(-c6ccccc6)n5)cc5sc6ccccc6c45)cc3)c3cccc(-c4ccccc4)c3)c2)cc1 ; c1ccc(-c2ccc(N(c3ccc4ccccc4c3)c3cc(-c4nc(-c5ccccc5)nc(-c5ccccc5)n4)c4c(c3)sc3ccccc34)cc2)cc1]

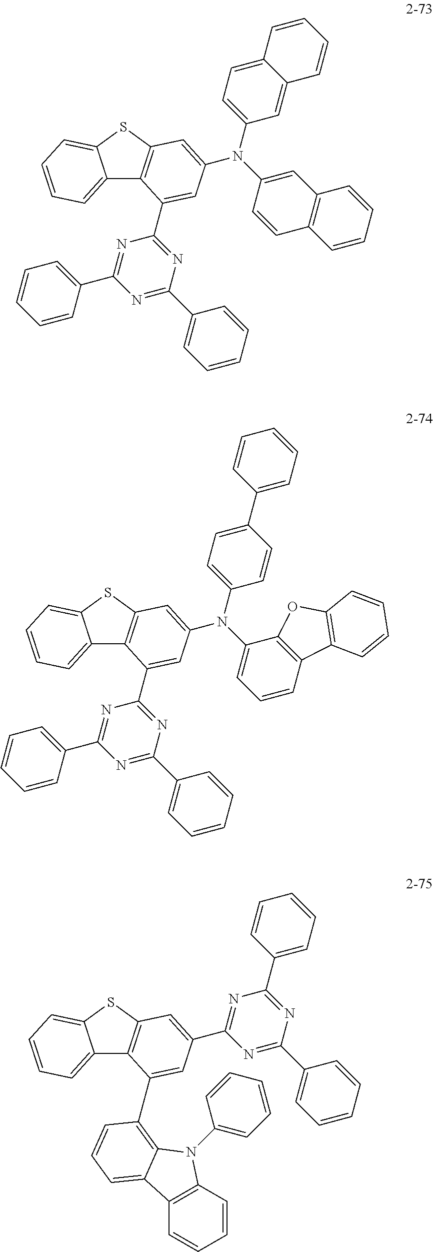 [SMILES: c1ccc(-c2nc(-c3ccccc3)nc(-c3cc(N(c4ccc5ccccc5c4)c4ccc5ccccc5c4)cc4sc5ccccc5c34)n2)cc1 ; c1ccc(-c2ccc(N(c3cc(-c4nc(-c5ccccc5)nc(-c5ccccc5)n4)c4c(c3)sc3ccccc34)c3cccc4c3oc3ccccc34)cc2)cc1 ; c1ccc(-c2nc(-c3ccccc3)nc(-c3cc(-c4cccc5c6ccccc6n(-c6ccccc6)c45)c4c(c3)sc3ccccc34)n2)cc1]

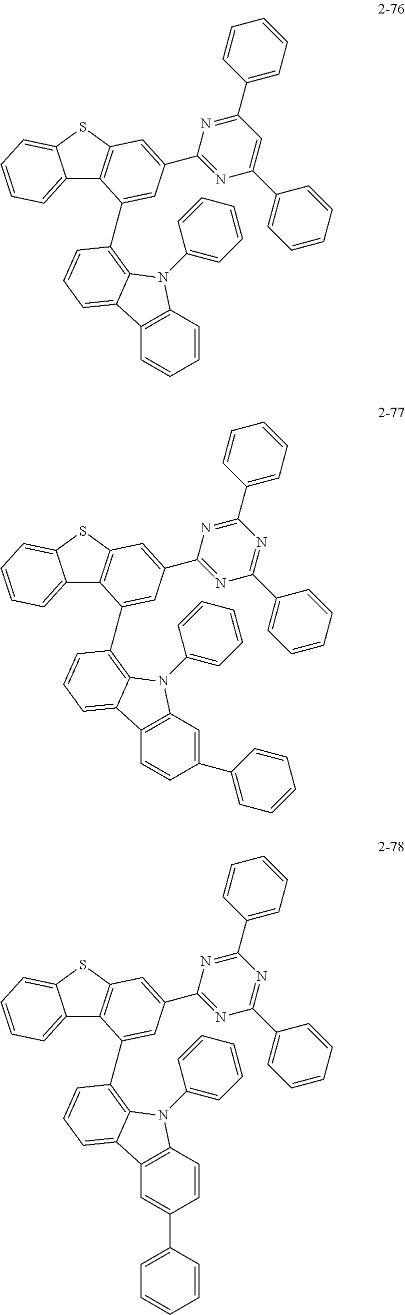

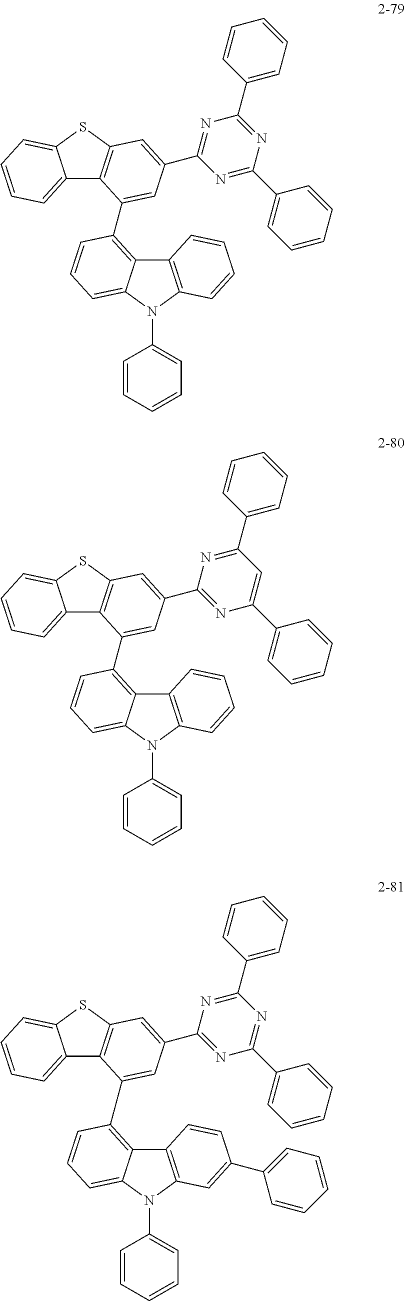 [SMILES: c1ccc(-c2nc(-c3ccccc3)nc(-c3cc(-c4cccc5c4c4ccccc4n5-c4ccccc4)c4c(c3)sc3ccccc34)n2)cc1 ; c1ccc(-c2cc(-c3ccccc3)nc(-c3cc(-c4cccc5c4c4ccccc4n5-c4ccccc4)c4c(c3)sc3ccccc34)n2)cc1 ; c1ccc(-c2ccc3c4c(-c5cc(-c6nc(-c7ccccc7)nc(-c7ccccc7)n6)cc6sc7ccccc7c56)cccc4n(-c4ccccc4)c3c2)cc1]

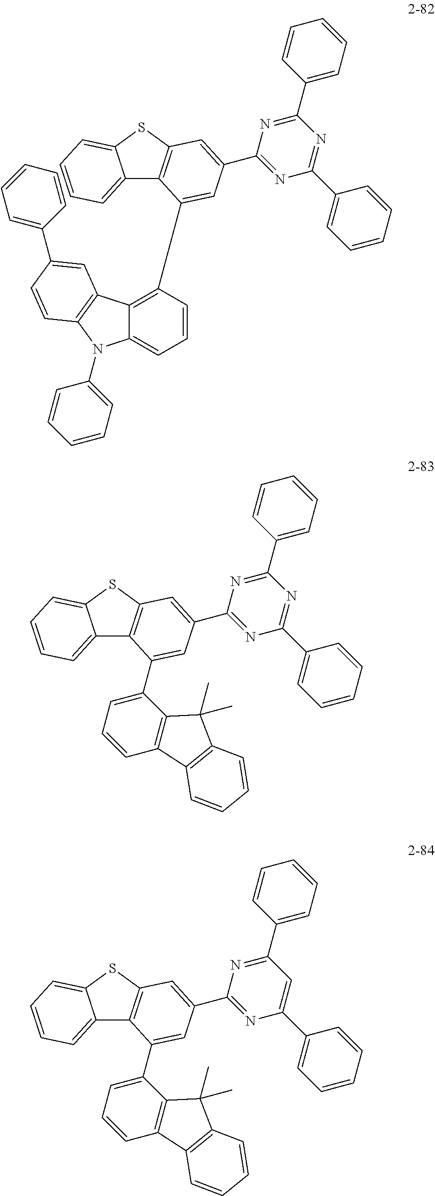 [SMILES: c1ccc(-c2ccc3c(c2)c2c(-c4cc(-c5nc(-c6ccccc6)nc(-c6ccccc6)n5)cc5sc6ccccc6c45)cccc2n3-c2ccccc2)cc1 ; CC1(C)c2ccccc2-c2cccc(-c3cc(-c4nc(-c5ccccc5)nc(-c5ccccc5)n4)cc4sc5ccccc5c34)c21 ; CC1(C)c2ccccc2-c2cccc(-c3cc(-c4nc(-c5ccccc5)cc(-c5ccccc5)n4)cc4sc5ccccc5c34)c21]

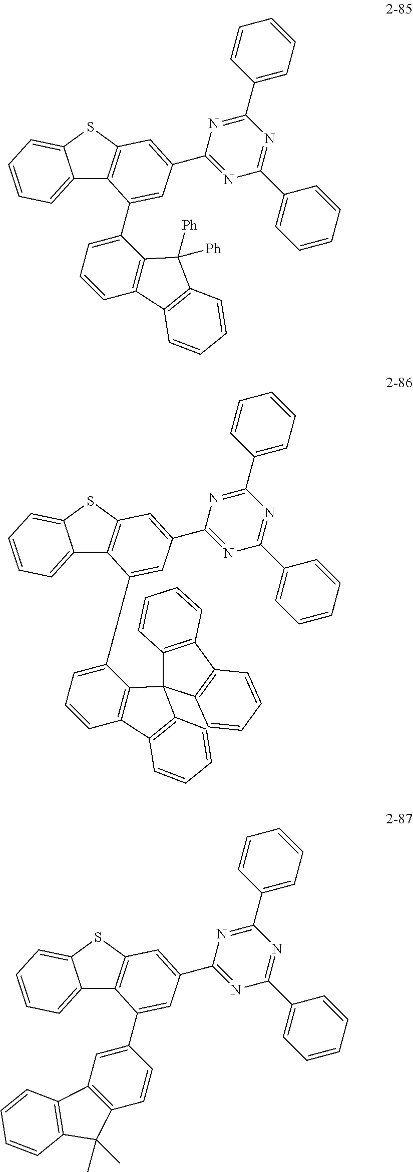 [SMILES: c1ccc(-c2nc(-c3ccccc3)nc(-c3cc(-c4cccc5c4C(c4ccccc4)(c4ccccc4)c4ccccc4-5)c4c(c3)sc3ccccc34)n2)cc1 ; c1ccc(-c2nc(-c3ccccc3)nc(-c3cc(-c4cccc5c4C4(c6ccccc6-c6ccccc64)c4ccccc4-5)c4c(c3)sc3ccccc34)n2)cc1 ; CC1(C)c2ccccc2-c2cc(-c3cc(-c4nc(-c5ccccc5)nc(-c5ccccc5)n4)cc4sc5ccccc5c34)ccc21]

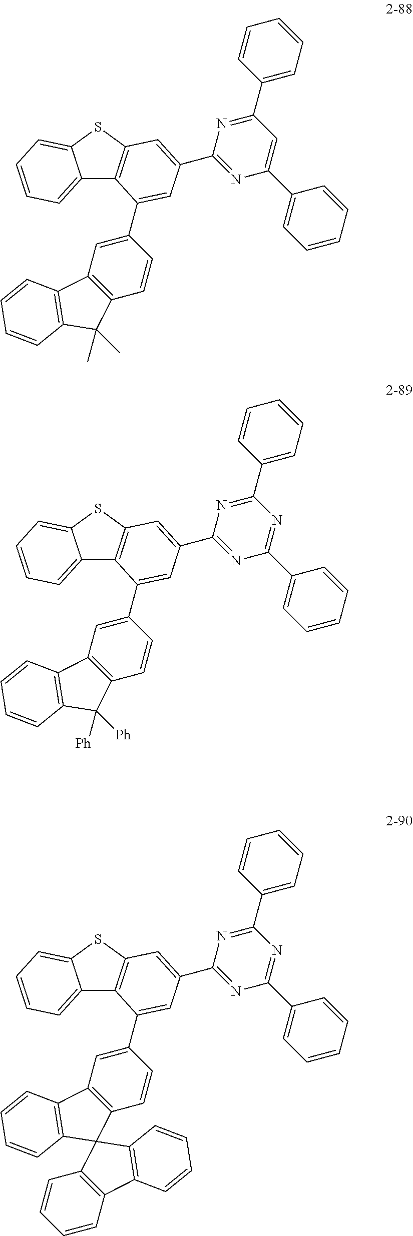 [SMILES: CC1(C)c2ccccc2-c2cc(-c3cc(-c4nc(-c5ccccc5)cc(-c5ccccc5)n4)cc4sc5ccccc5c34)ccc21 ; c1ccc(-c2nc(-c3ccccc3)nc(-c3cc(-c4ccc5c(c4)-c4ccccc4C5(c4ccccc4)c4ccccc4)c4c(c3)sc3ccccc34)n2)cc1 ; c1ccc(-c2nc(-c3ccccc3)nc(-c3cc(-c4ccc5c(c4)-c4ccccc4C54c5ccccc5-c5ccccc54)c4c(c3)sc3ccccc34)n2)cc1]

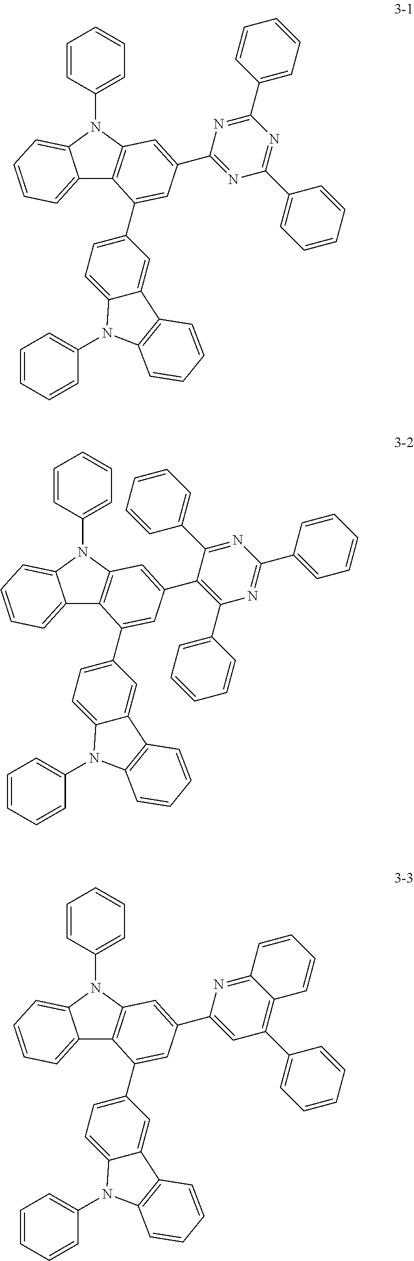 [SMILES: c1ccc(-c2nc(-c3ccccc3)nc(-c3cc(-c4ccc5c(c4)c4ccccc4n5-c4ccccc4)c4c5ccccc5n(-c5ccccc5)c4c3)n2)cc1 ; c1ccc(-c2nc(-c3ccccc3)c(-c3cc(-c4ccc5c(c4)c4ccccc4n5-c4ccccc4)c4c5ccccc5n(-c5ccccc5)c4c3)c(-c3ccccc3)n2)cc1 ; c1ccc(-c2cc(-c3cc(-c4ccc5c(c4)c4ccccc4n5-c4ccccc4)c4c5ccccc5n(-c5ccccc5)c4c3)nc3ccccc23)cc1]

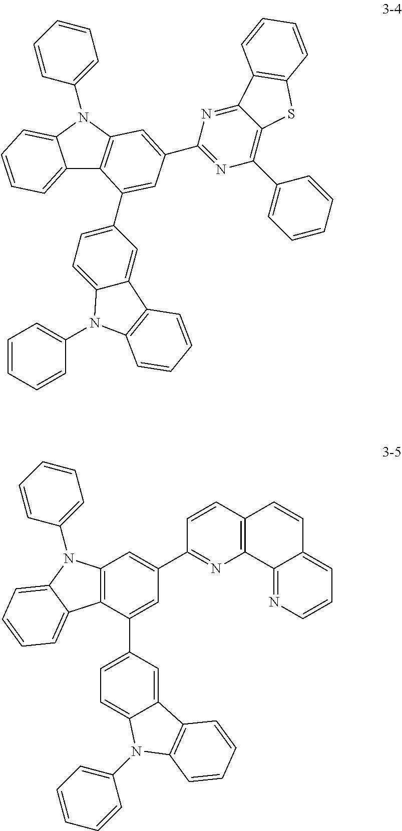 [SMILES: c1ccc(-c2nc(-c3cc(-c4ccc5c(c4)c4ccccc4n5-c4ccccc4)c4c5ccccc5n(-c5ccccc5)c4c3)nc3c2sc2ccccc23)cc1 ; c1ccc(-n2c3ccccc3c3cc(-c4cc(-c5ccc6ccc7cccnc7c6n5)cc5c4c4ccccc4n5-c4ccccc4)ccc32)cc1]

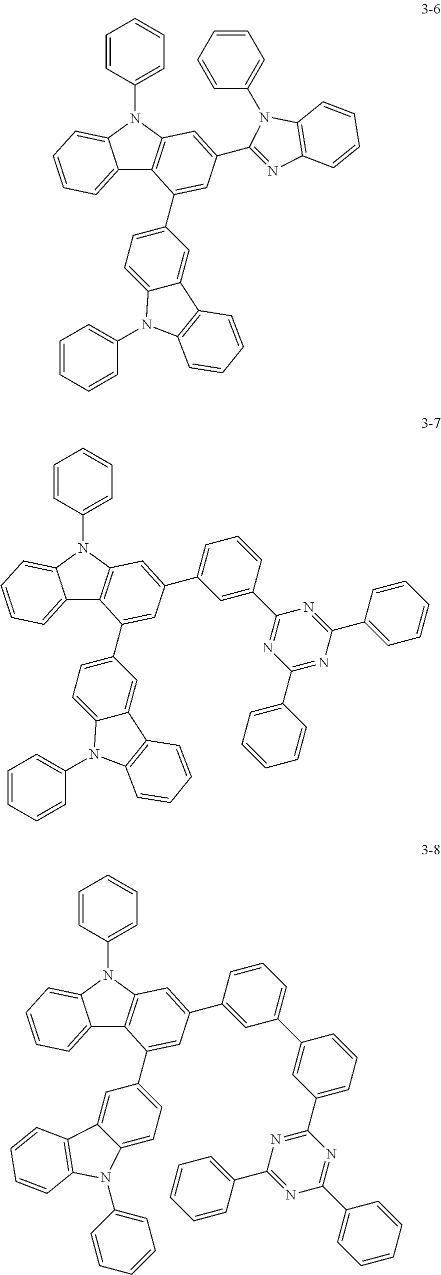 [SMILES: c1ccc(-n2c(-c3cc(-c4ccc5c(c4)c4ccccc4n5-c4ccccc4)c4c5ccccc5n(-c5ccccc5)c4c3)nc3ccccc32)cc1 ; c1ccc(-c2nc(-c3ccccc3)nc(-c3cccc(-c4cc(-c5ccc6c(c5)c5ccccc5n6-c5ccccc5)c5c6ccccc6n(-c6ccccc6)c5c4)c3)n2)cc1 ; c1ccc(-c2nc(-c3ccccc3)nc(-c3cccc(-c4cccc(-c5cc(-c6ccc7c(c6)c6ccccc6n7-c6ccccc6)c6c7ccccc7n(-c7ccccc7)c6c5)c4)c3)n2)cc1]

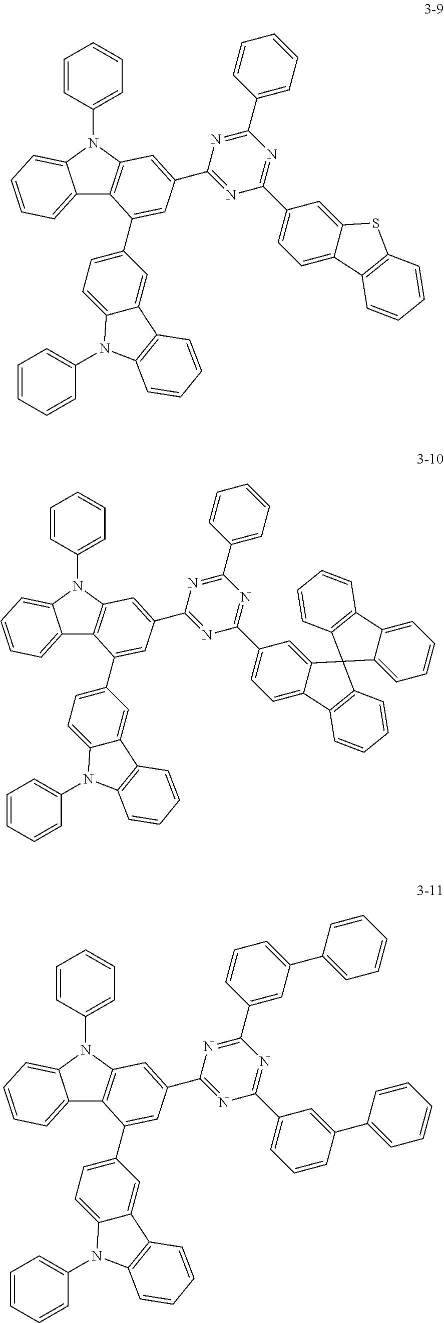 [SMILES: c1ccc(-c2nc(-c3ccc4c(c3)sc3ccccc34)nc(-c3cc(-c4ccc5c(c4)c4ccccc4n5-c4ccccc4)c4c5ccccc5n(-c5ccccc5)c4c3)n2)cc1 ; c1ccc(-c2nc(-c3ccc4c(c3)C3(c5ccccc5-c5ccccc53)c3ccccc3-4)nc(-c3cc(-c4ccc5c(c4)c4ccccc4n5-c4ccccc4)c4c5ccccc5n(-c5ccccc5)c4c3)n2)cc1 ; c1ccc(-c2cccc(-c3nc(-c4cccc(-c5ccccc5)c4)nc(-c4cc(-c5ccc6c(c5)c5ccccc5n6-c5ccccc5)c5c6ccccc6n(-c6ccccc6)c5c4)n3)c2)cc1]

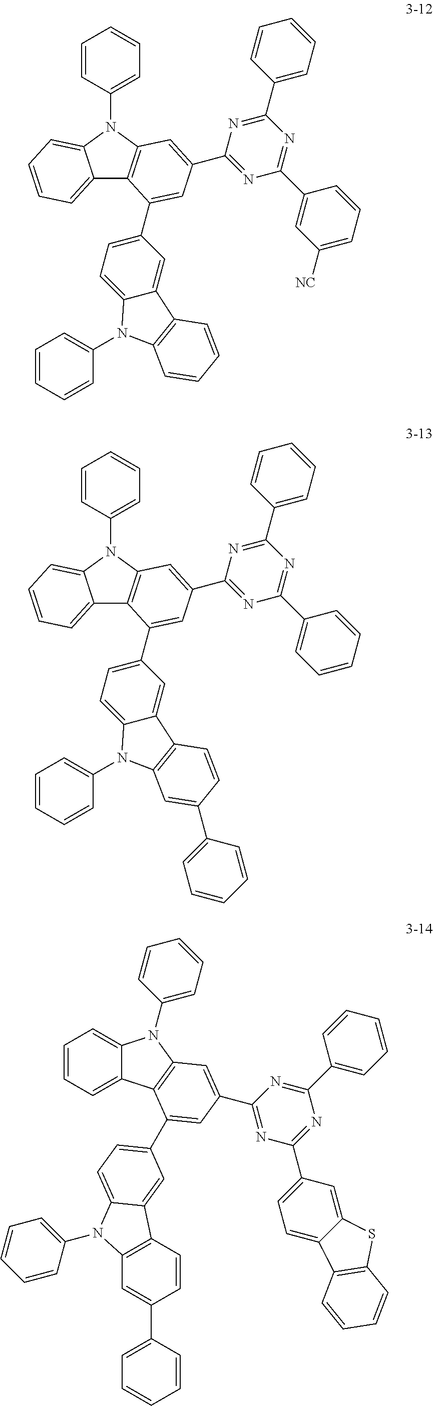

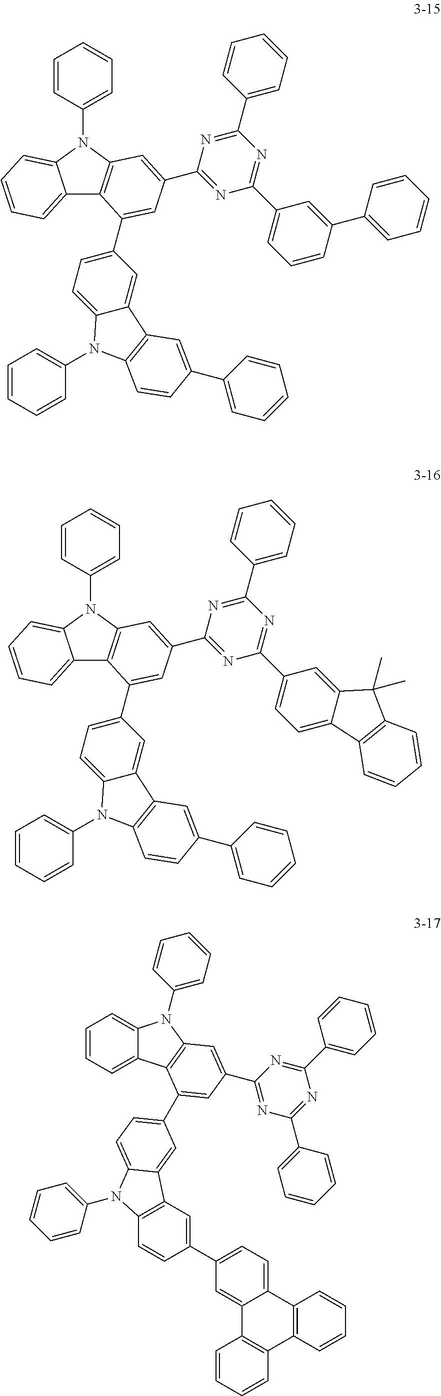 [SMILES: c1ccc(-c2cccc(-c3nc(-c4ccccc4)nc(-c4cc(-c5ccc6c(c5)c5cc(-c7ccccc7)ccc5n6-c5ccccc5)c5c6ccccc6n(-c6ccccc6)c5c4)n3)c2)cc1 ; CC1(C)c2ccccc2-c2ccc(-c3nc(-c4ccccc4)nc(-c4cc(-c5ccc6c(c5)c5cc(-c7ccccc7)ccc5n6-c5ccccc5)c5c6ccccc6n(-c6ccccc6)c5c4)n3)cc21 ; c1ccc(-c2nc(-c3ccccc3)nc(-c3cc(-c4ccc5c(c4)c4cc(-c6ccc7c8ccccc8c8ccccc8c7c6)ccc4n5-c4ccccc4)c4c5ccccc5n(-c5ccccc5)c4c3)n2)cc1]

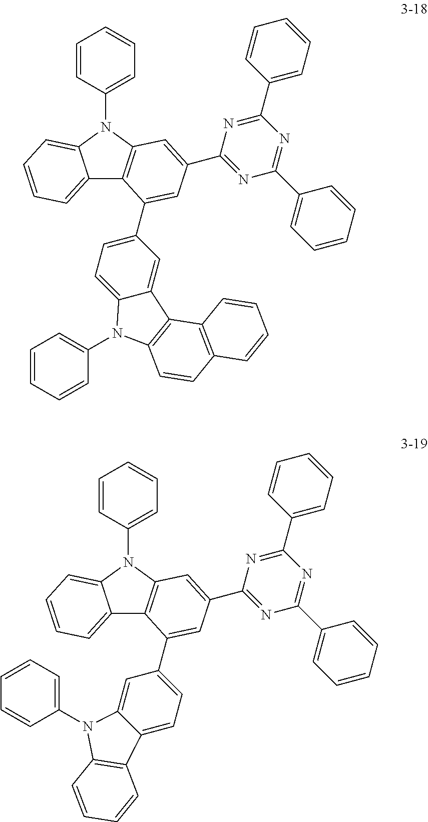 [SMILES: c1ccc(-c2nc(-c3ccccc3)nc(-c3cc(-c4ccc5c(c4)c4c6ccccc6ccc4n5-c4ccccc4)c4c5ccccc5n(-c5ccccc5)c4c3)n2)cc1 ; c1ccc(-c2nc(-c3ccccc3)nc(-c3cc(-c4ccc5c6ccccc6n(-c6ccccc6)c5c4)c4c5ccccc5n(-c5ccccc5)c4c3)n2)cc1]

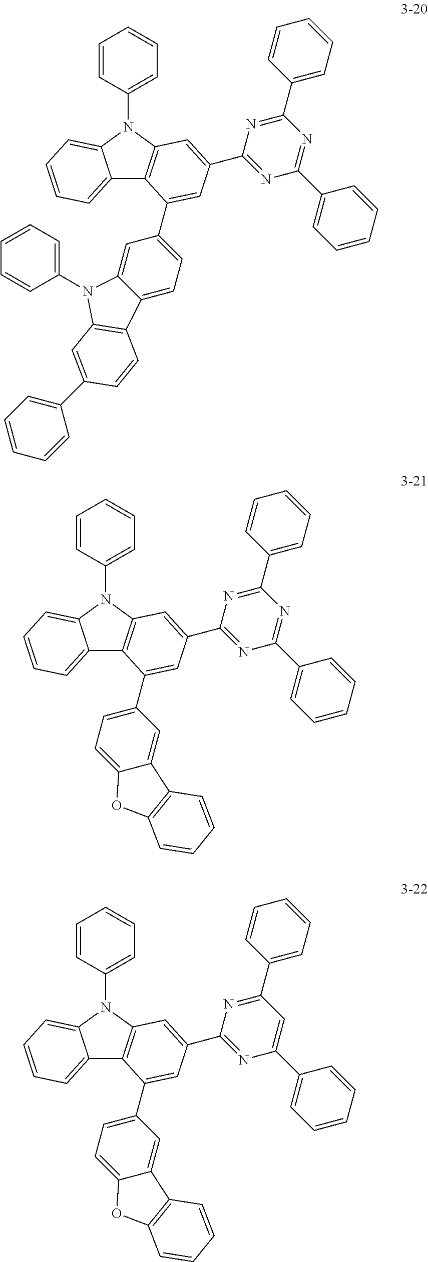 [SMILES: c1ccc(-c2ccc3c4ccc(-c5cc(-c6nc(-c7ccccc7)nc(-c7ccccc7)n6)cc6c5c5ccccc5n6-c5ccccc5)cc4n(-c4ccccc4)c3c2)cc1 ; c1ccc(-c2nc(-c3ccccc3)nc(-c3cc(-c4ccc5oc6ccccc6c5c4)c4c5ccccc5n(-c5ccccc5)c4c3)n2)cc1 ; c1ccc(-c2cc(-c3ccccc3)nc(-c3cc(-c4ccc5oc6ccccc6c5c4)c4c5ccccc5n(-c5ccccc5)c4c3)n2)cc1]

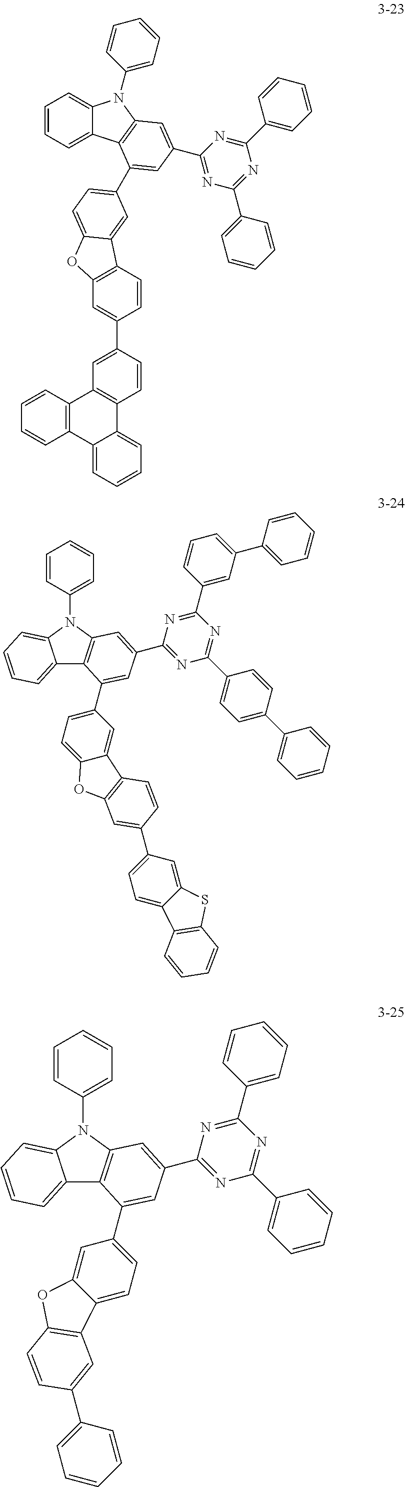 [SMILES: c1ccc(-c2nc(-c3ccccc3)nc(-c3cc(-c4ccc5oc6cc(-c7ccc8c9ccccc9c9ccccc9c8c7)ccc6c5c4)c4c5ccccc5n(-c5ccccc5)c4c3)n2)cc1 ; c1ccc(-c2ccc(-c3nc(-c4cccc(-c5ccccc5)c4)nc(-c4cc(-c5ccc6oc7cc(-c8ccc9c(c8)sc8ccccc89)ccc7c6c5)c5c6ccccc6n(-c6ccccc6)c5c4)n3)cc2)cc1 ; c1ccc(-c2ccc3oc4cc(-c5cc(-c6nc(-c7ccccc7)nc(-c7ccccc7)n6)cc6c5c5ccccc5n6-c5ccccc5)ccc4c3c2)cc1]

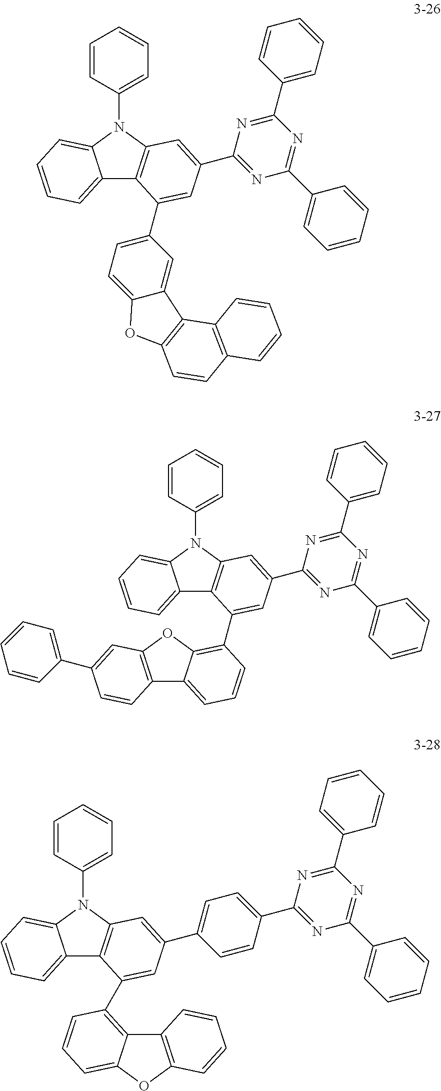 [SMILES: c1ccc(-c2nc(-c3ccccc3)nc(-c3cc(-c4ccc5oc6ccc7ccccc7c6c5c4)c4c5ccccc5n(-c5ccccc5)c4c3)n2)cc1 ; c1ccc(-c2ccc3c(c2)oc2c(-c4cc(-c5nc(-c6ccccc6)nc(-c6ccccc6)n5)cc5c4c4ccccc4n5-c4ccccc4)cccc23)cc1 ; c1ccc(-c2nc(-c3ccccc3)nc(-c3ccc(-c4cc(-c5cccc6oc7ccccc7c56)c5c6ccccc6n(-c6ccccc6)c5c4)cc3)n2)cc1]

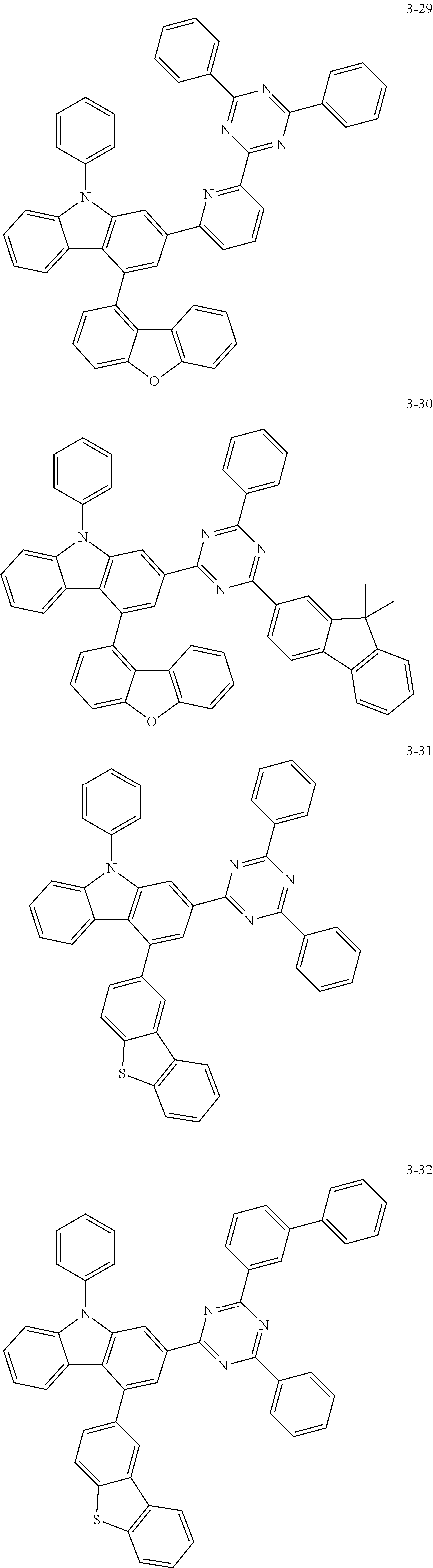 [SMILES: c1ccc(-c2nc(-c3ccccc3)nc(-c3cccc(-c4cc(-c5cccc6oc7ccccc7c56)c5c6ccccc6n(-c6ccccc6)c5c4)n3)n2)cc1 ; CC1(C)c2ccccc2-c2ccc(-c3nc(-c4ccccc4)nc(-c4cc(-c5cccc6oc7ccccc7c56)c5c6ccccc6n(-c6ccccc6)c5c4)n3)cc21 ; c1ccc(-c2nc(-c3ccccc3)nc(-c3cc(-c4ccc5sc6ccccc6c5c4)c4c5ccccc5n(-c5ccccc5)c4c3)n2)cc1 ; c1ccc(-c2cccc(-c3nc(-c4ccccc4)nc(-c4cc(-c5ccc6sc7ccccc7c6c5)c5c6ccccc6n(-c6ccccc6)c5c4)n3)c2)cc1]

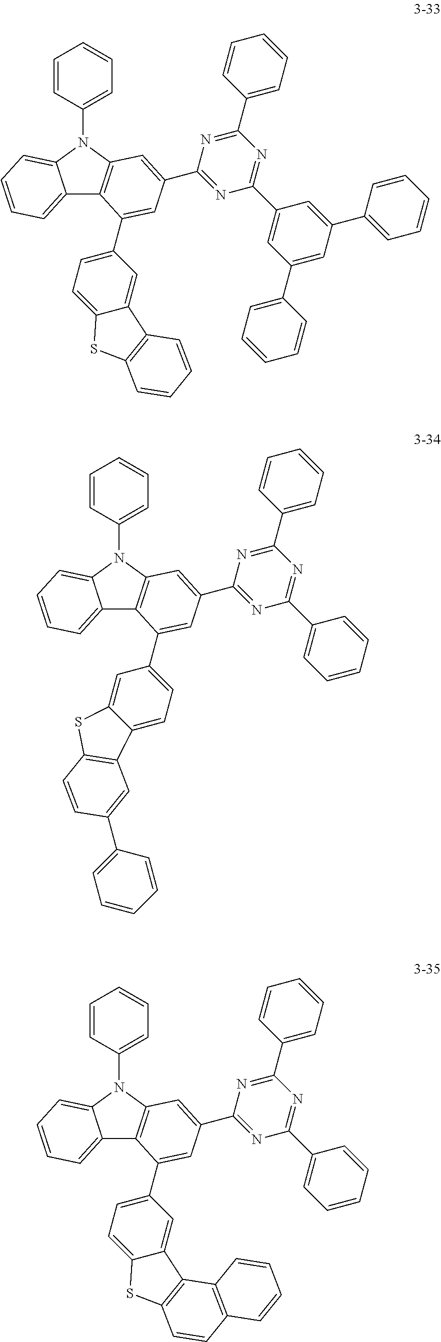 [SMILES: c1ccc(-c2cc(-c3ccccc3)cc(-c3nc(-c4ccccc4)nc(-c4cc(-c5ccc6sc7ccccc7c6c5)c5c6ccccc6n(-c6ccccc6)c5c4)n3)c2)cc1 ; c1ccc(-c2ccc3sc4cc(-c5cc(-c6nc(-c7ccccc7)nc(-c7ccccc7)n6)cc6c5c5ccccc5n6-c5ccccc5)ccc4c3c2)cc1 ; c1ccc(-c2nc(-c3ccccc3)nc(-c3cc(-c4ccc5sc6ccc7ccccc7c6c5c4)c4c5ccccc5n(-c5ccccc5)c4c3)n2)cc1]

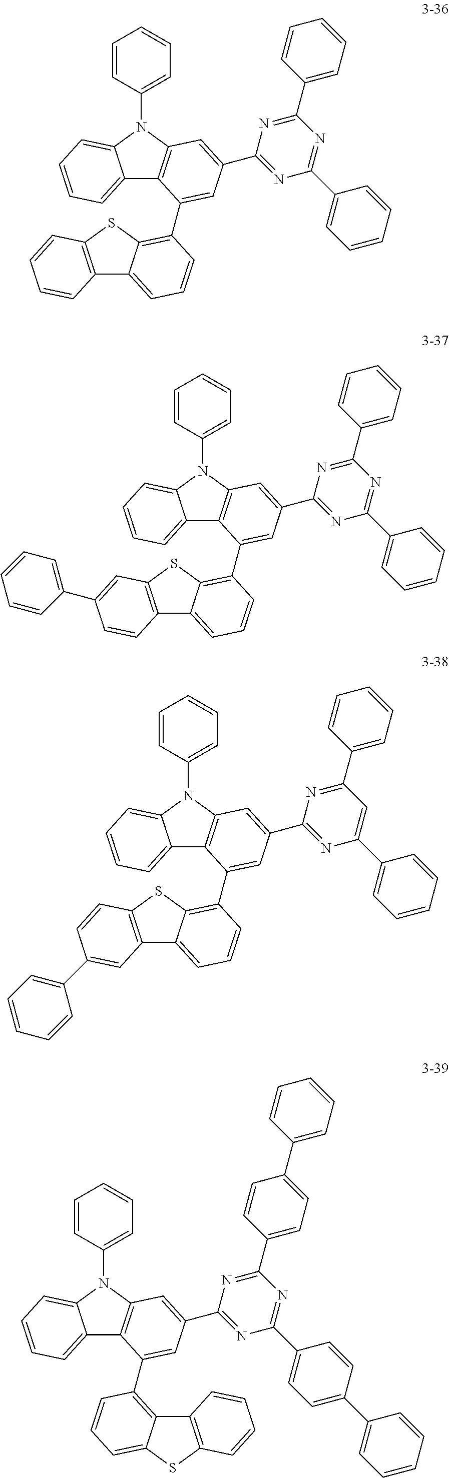 [SMILES: c1ccc(-c2nc(-c3ccccc3)nc(-c3cc(-c4cccc5c4sc4ccccc45)c4c5ccccc5n(-c5ccccc5)c4c3)n2)cc1 ; c1ccc(-c2ccc3c(c2)sc2c(-c4cc(-c5nc(-c6ccccc6)nc(-c6ccccc6)n5)cc5c4c4ccccc4n5-c4ccccc4)cccc23)cc1 ; c1ccc(-c2ccc3sc4c(-c5cc(-c6nc(-c7ccccc7)cc(-c7ccccc7)n6)cc6c5c5ccccc5n6-c5ccccc5)cccc4c3c2)cc1 ; c1ccc(-c2ccc(-c3nc(-c4ccc(-c5ccccc5)cc4)nc(-c4cc(-c5cccc6sc7ccccc7c56)c5c6ccccc6n(-c6ccccc6)c5c4)n3)cc2)cc1]

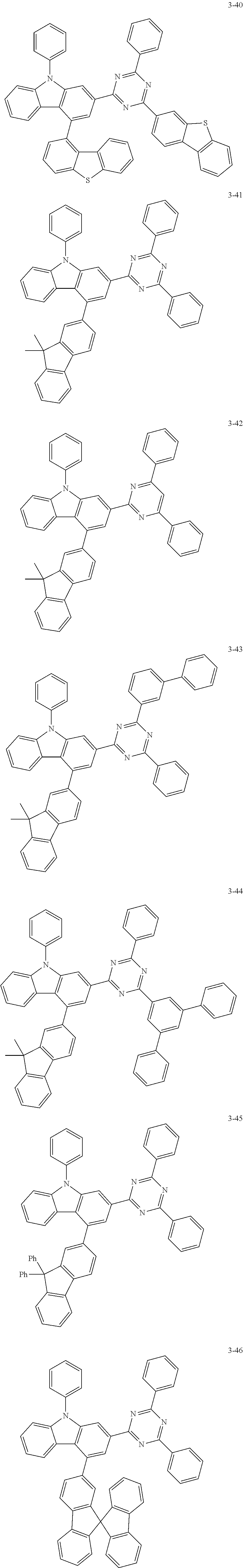

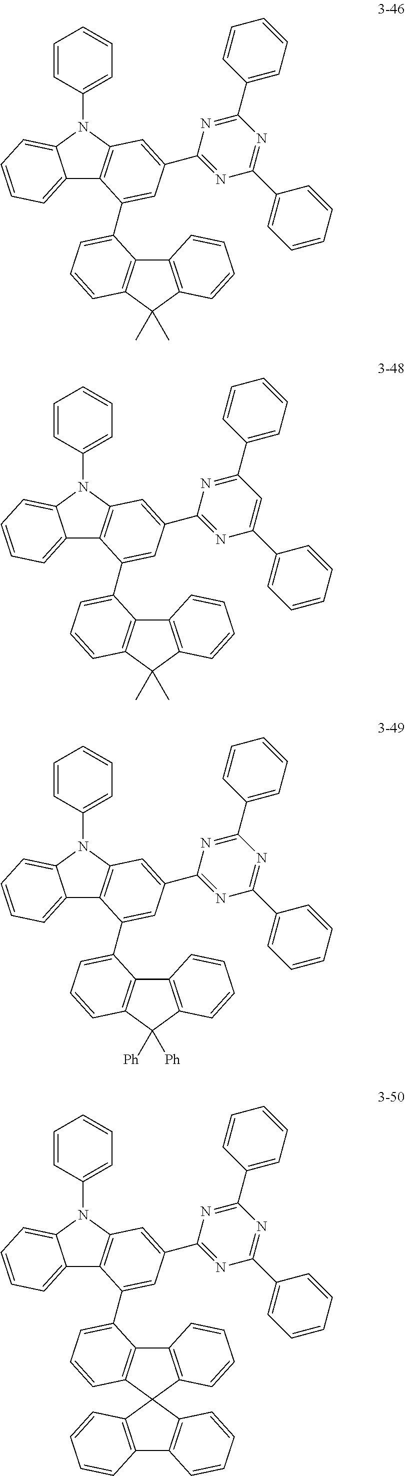 [SMILES: CC1(C)c2ccccc2-c2c(-c3cc(-c4nc(-c5ccccc5)nc(-c5ccccc5)n4)cc4c3c3ccccc3n4-c3ccccc3)cccc21 ; CC1(C)c2ccccc2-c2c(-c3cc(-c4nc(-c5ccccc5)cc(-c5ccccc5)n4)cc4c3c3ccccc3n4-c3ccccc3)cccc21 ; c1ccc(-c2nc(-c3ccccc3)nc(-c3cc(-c4cccc5c4-c4ccccc4C5(c4ccccc4)c4ccccc4)c4c5ccccc5n(-c5ccccc5)c4c3)n2)cc1 ; c1ccc(-c2nc(-c3ccccc3)nc(-c3cc(-c4cccc5c4-c4ccccc4C54c5ccccc5-c5ccccc54)c4c5ccccc5n(-c5ccccc5)c4c3)n2)cc1]

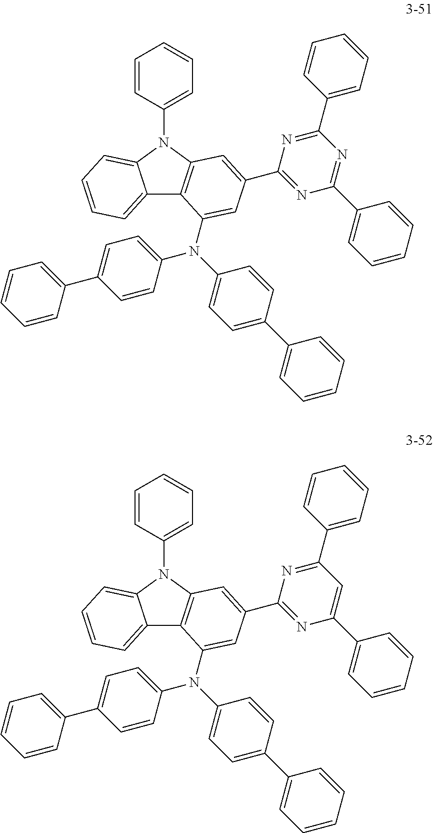 [SMILES: c1ccc(-c2ccc(N(c3ccc(-c4ccccc4)cc3)c3cc(-c4nc(-c5ccccc5)nc(-c5ccccc5)n4)cc4c3c3ccccc3n4-c3ccccc3)cc2)cc1 ; c1ccc(-c2ccc(N(c3ccc(-c4ccccc4)cc3)c3cc(-c4nc(-c5ccccc5)cc(-c5ccccc5)n4)cc4c3c3ccccc3n4-c3ccccc3)cc2)cc1]

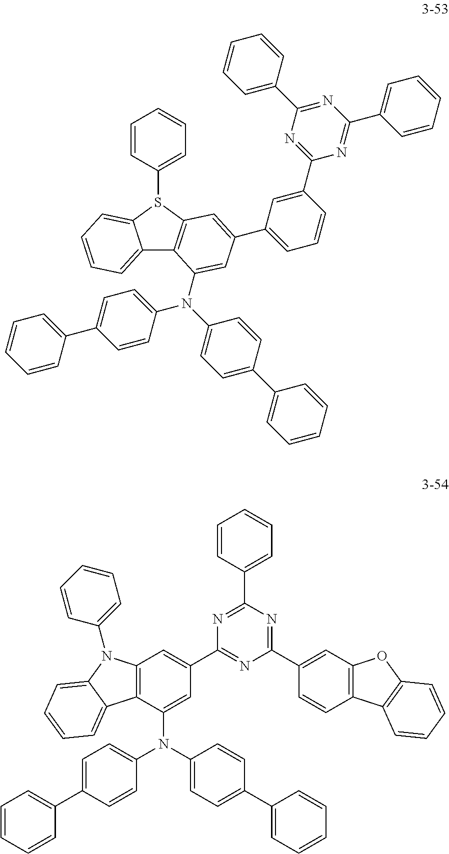

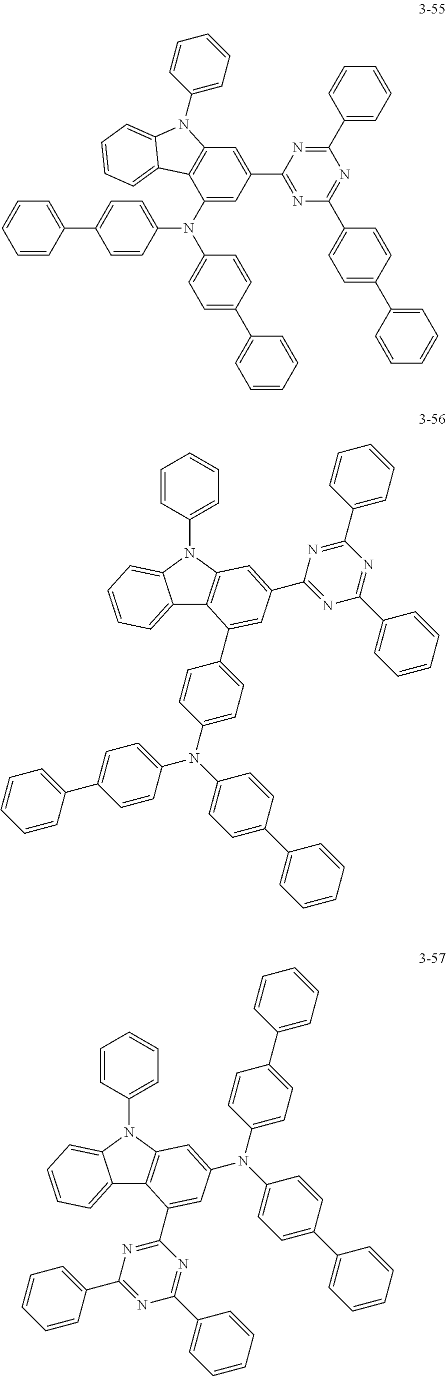 [SMILES: c1ccc(-c2ccc(-c3nc(-c4ccccc4)nc(-c4cc(N(c5ccc(-c6ccccc6)cc5)c5ccc(-c6ccccc6)cc5)c5c6ccccc6n(-c6ccccc6)c5c4)n3)cc2)cc1 ; c1ccc(-c2ccc(N(c3ccc(-c4ccccc4)cc3)c3ccc(-c4cc(-c5nc(-c6ccccc6)nc(-c6ccccc6)n5)cc5c4c4ccccc4n5-c4ccccc4)cc3)cc2)cc1 ; c1ccc(-c2ccc(N(c3ccc(-c4ccccc4)cc3)c3cc(-c4nc(-c5ccccc5)nc(-c5ccccc5)n4)c4c5ccccc5n(-c5ccccc5)c4c3)cc2)cc1]

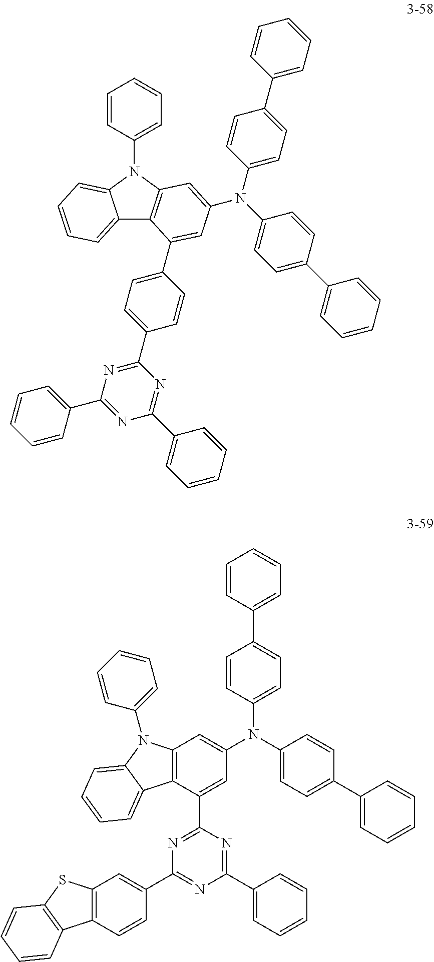 [SMILES: c1ccc(-c2ccc(N(c3ccc(-c4ccccc4)cc3)c3cc(-c4ccc(-c5nc(-c6ccccc6)nc(-c6ccccc6)n5)cc4)c4c5ccccc5n(-c5ccccc5)c4c3)cc2)cc1 ; c1ccc(-c2ccc(N(c3ccc(-c4ccccc4)cc3)c3cc(-c4nc(-c5ccccc5)nc(-c5ccc6c(c5)sc5ccccc56)n4)c4c5ccccc5n(-c5ccccc5)c4c3)cc2)cc1]

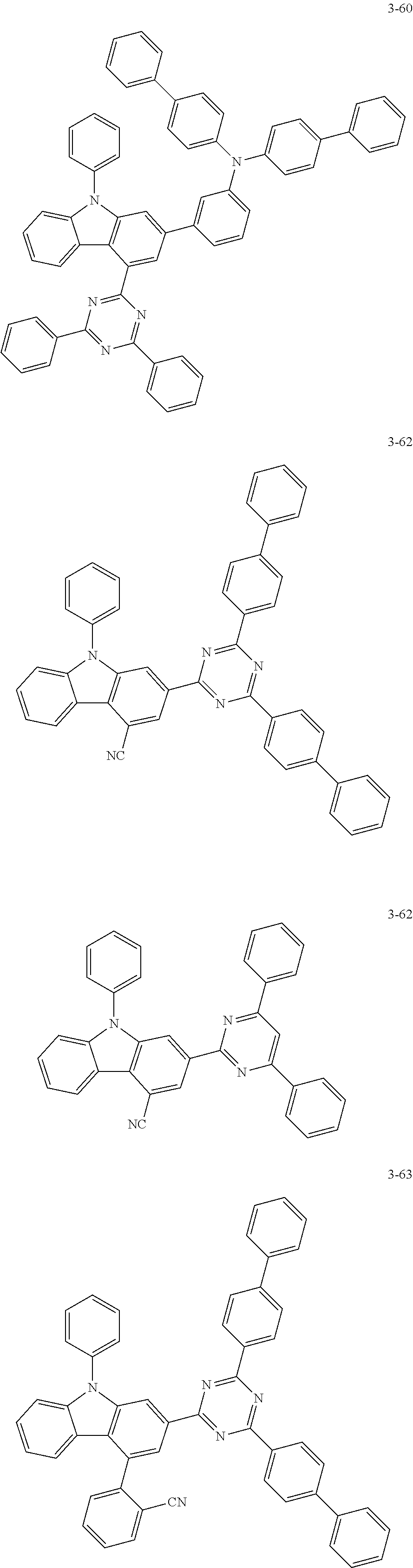 [SMILES: c1ccc(-c2ccc(N(c3ccc(-c4ccccc4)cc3)c3cccc(-c4cc(-c5nc(-c6ccccc6)nc(-c6ccccc6)n5)c5c6ccccc6n(-c6ccccc6)c5c4)c3)cc2)cc1 ; N#Cc1cc(-c2nc(-c3ccc(-c4ccccc4)cc3)nc(-c3ccc(-c4ccccc4)cc3)n2)cc2c1c1ccccc1n2-c1ccccc1 ; N#Cc1cc(-c2nc(-c3ccccc3)cc(-c3ccccc3)n2)cc2c1c1ccccc1n2-c1ccccc1 ; N#Cc1ccccc1-c1cc(-c2nc(-c3ccc(-c4ccccc4)cc3)nc(-c3ccc(-c4ccccc4)cc3)n2)cc2c1c1ccccc1n2-c1ccccc1]

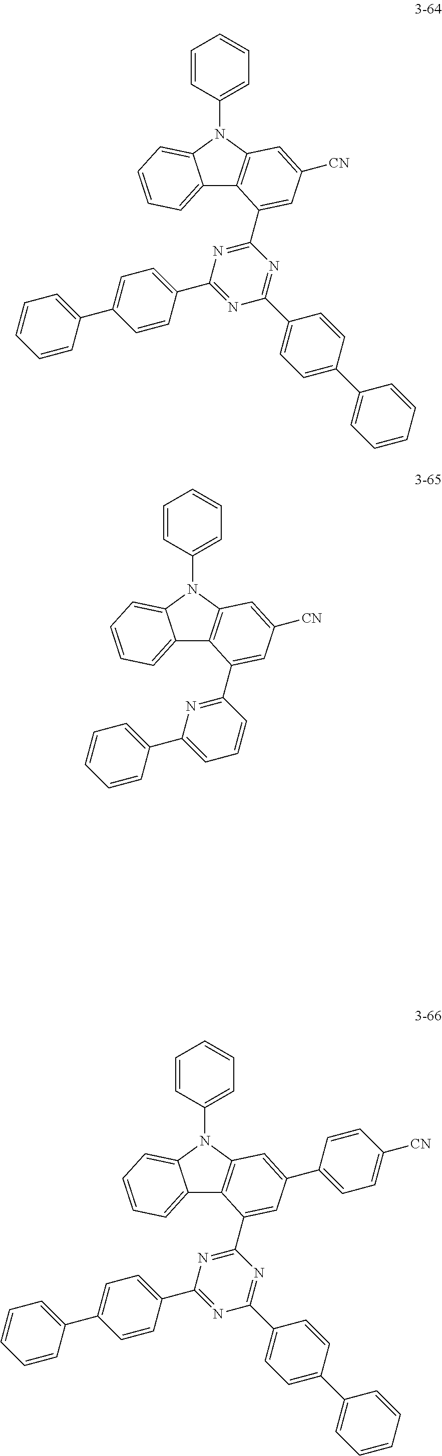 [SMILES: N#Cc1cc(-c2nc(-c3ccc(-c4ccccc4)cc3)nc(-c3ccc(-c4ccccc4)cc3)n2)c2c3ccccc3n(-c3ccccc3)c2c1 ; N#Cc1cc(-c2cccc(-c3ccccc3)n2)c2c3ccccc3n(-c3ccccc3)c2c1 ; N#Cc1ccc(-c2cc(-c3nc(-c4ccc(-c5ccccc5)cc4)nc(-c4ccc(-c5ccccc5)cc4)n3)c3c4ccccc4n(-c4ccccc4)c3c2)cc1]

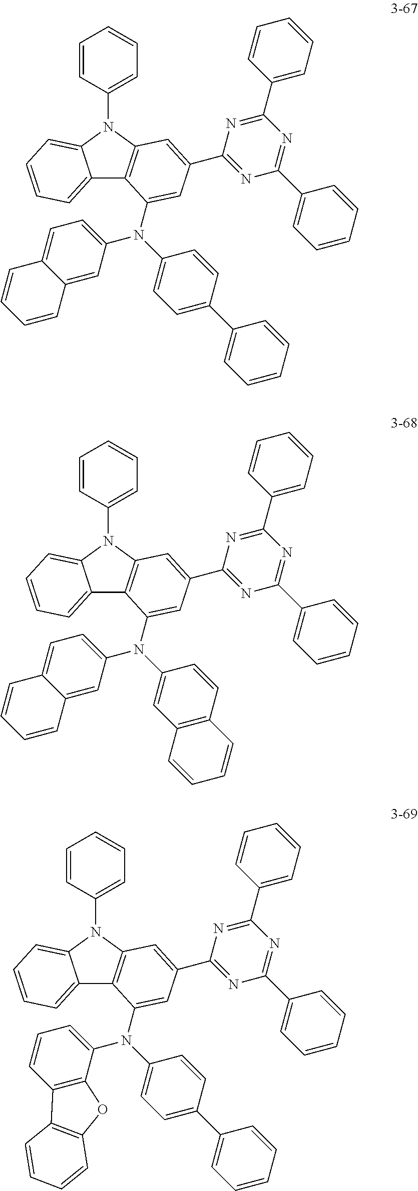 [SMILES: c1ccc(-c2ccc(N(c3ccc4ccccc4c3)c3cc(-c4nc(-c5ccccc5)nc(-c5ccccc5)n4)cc4c3c3ccccc3n4-c3ccccc3)cc2)cc1 ; c1ccc(-c2nc(-c3ccccc3)nc(-c3cc(N(c4ccc5ccccc5c4)c4ccc5ccccc5c4)c4c5ccccc5n(-c5ccccc5)c4c3)n2)cc1 ; c1ccc(-c2ccc(N(c3cccc4c3oc3ccccc34)c3cc(-c4nc(-c5ccccc5)nc(-c5ccccc5)n4)cc4c3c3ccccc3n4-c3ccccc3)cc2)cc1]

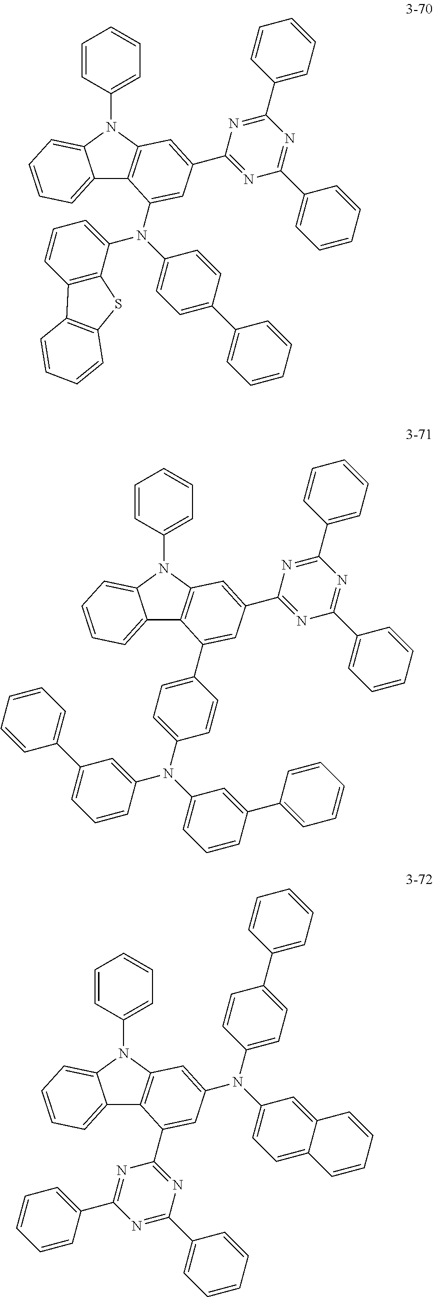 [SMILES: c1ccc(-c2ccc(N(c3cccc4c3sc3ccccc34)c3cc(-c4nc(-c5ccccc5)nc(-c5ccccc5)n4)cc4c3c3ccccc3n4-c3ccccc3)cc2)cc1 ; c1ccc(-c2cccc(N(c3ccc(-c4cc(-c5nc(-c6ccccc6)nc(-c6ccccc6)n5)cc5c4c4ccccc4n5-c4ccccc4)cc3)c3cccc(-c4ccccc4)c3)c2)cc1 ; c1ccc(-c2ccc(N(c3ccc4ccccc4c3)c3cc(-c4nc(-c5ccccc5)nc(-c5ccccc5)n4)c4c5ccccc5n(-c5ccccc5)c4c3)cc2)cc1]

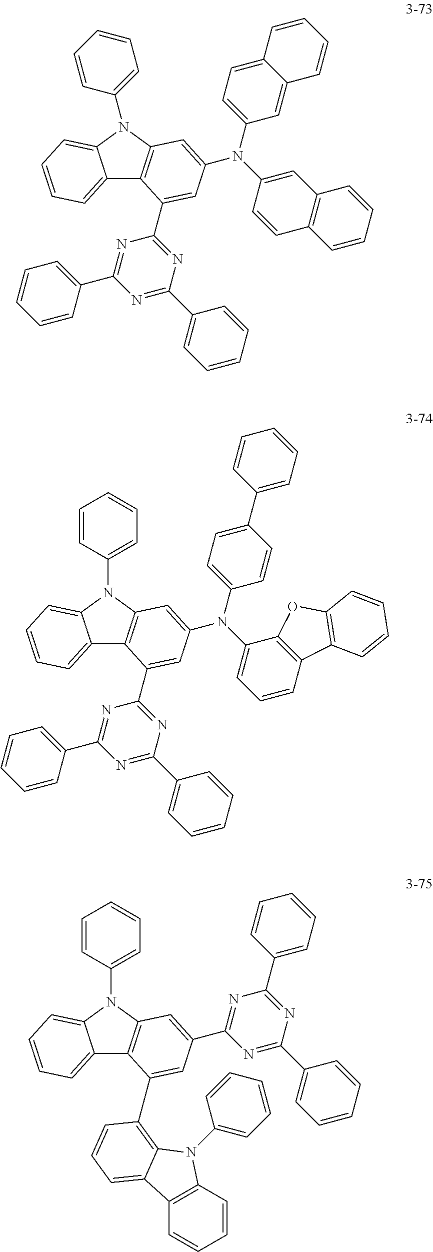 [SMILES: c1ccc(-c2nc(-c3ccccc3)nc(-c3cc(N(c4ccc5ccccc5c4)c4ccc5ccccc5c4)cc4c3c3ccccc3n4-c3ccccc3)n2)cc1 ; c1ccc(-c2ccc(N(c3cc(-c4nc(-c5ccccc5)nc(-c5ccccc5)n4)c4c5ccccc5n(-c5ccccc5)c4c3)c3cccc4c3oc3ccccc34)cc2)cc1 ; c1ccc(-c2nc(-c3ccccc3)nc(-c3cc(-c4cccc5c6ccccc6n(-c6ccccc6)c45)c4c5ccccc5n(-c5ccccc5)c4c3)n2)cc1]

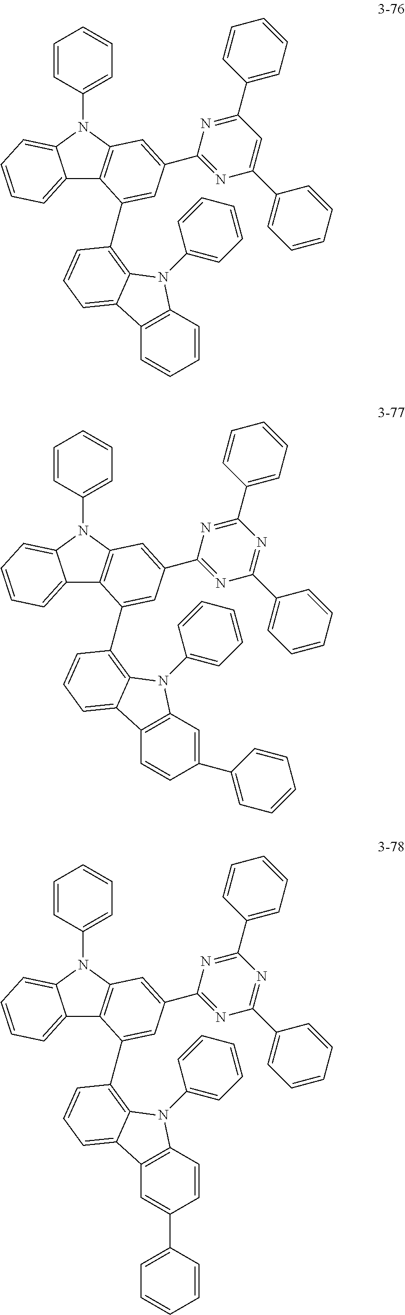

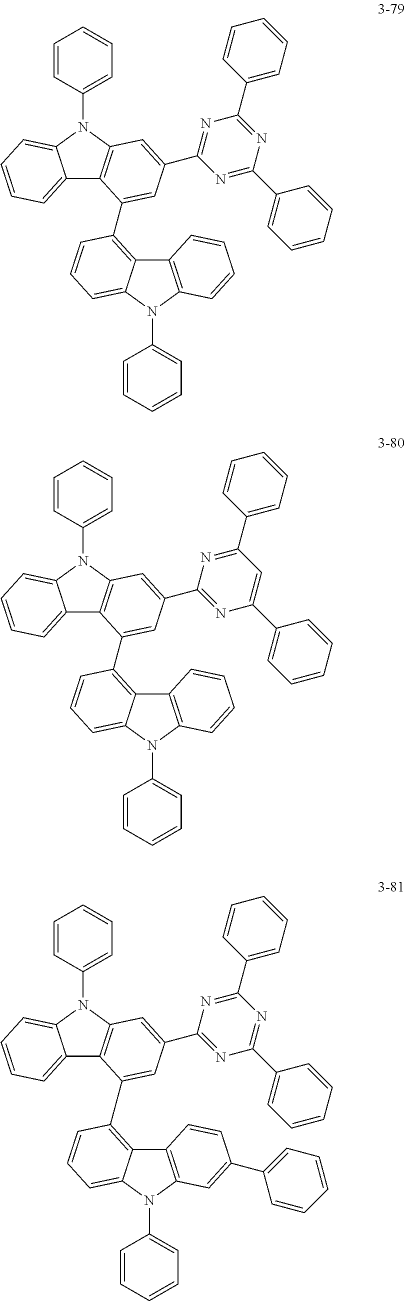 [SMILES: c1ccc(-c2nc(-c3ccccc3)nc(-c3cc(-c4cccc5c4c4ccccc4n5-c4ccccc4)c4c5ccccc5n(-c5ccccc5)c4c3)n2)cc1 ; c1ccc(-c2cc(-c3ccccc3)nc(-c3cc(-c4cccc5c4c4ccccc4n5-c4ccccc4)c4c5ccccc5n(-c5ccccc5)c4c3)n2)cc1 ; c1ccc(-c2ccc3c4c(-c5cc(-c6nc(-c7ccccc7)nc(-c7ccccc7)n6)cc6c5c5ccccc5n6-c5ccccc5)cccc4n(-c4ccccc4)c3c2)cc1]

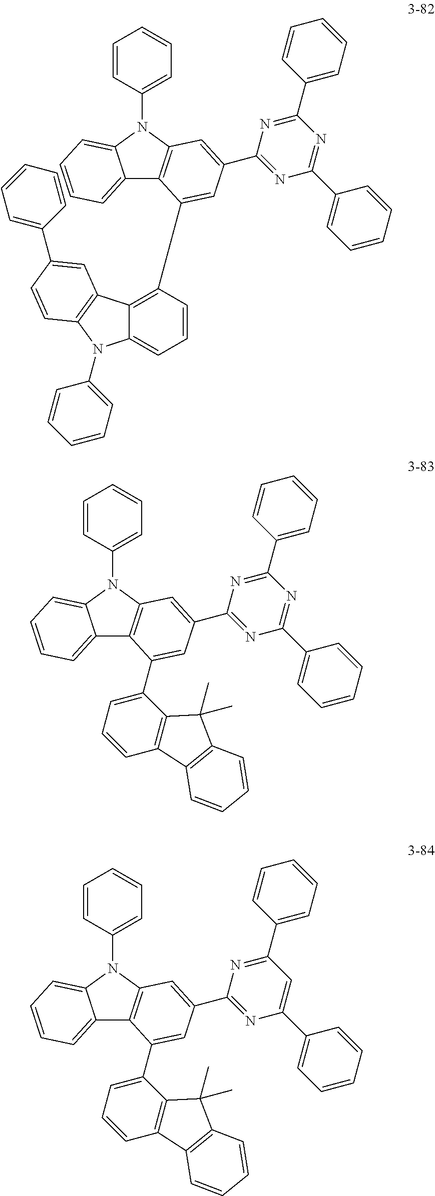 [SMILES: c1ccc(-c2ccc3c(c2)c2c(-c4cc(-c5nc(-c6ccccc6)nc(-c6ccccc6)n5)cc5c4c4ccccc4n5-c4ccccc4)cccc2n3-c2ccccc2)cc1 ; CC1(C)c2ccccc2-c2cccc(-c3cc(-c4nc(-c5ccccc5)nc(-c5ccccc5)n4)cc4c3c3ccccc3n4-c3ccccc3)c21 ; CC1(C)c2ccccc2-c2cccc(-c3cc(-c4nc(-c5ccccc5)cc(-c5ccccc5)n4)cc4c3c3ccccc3n4-c3ccccc3)c21]

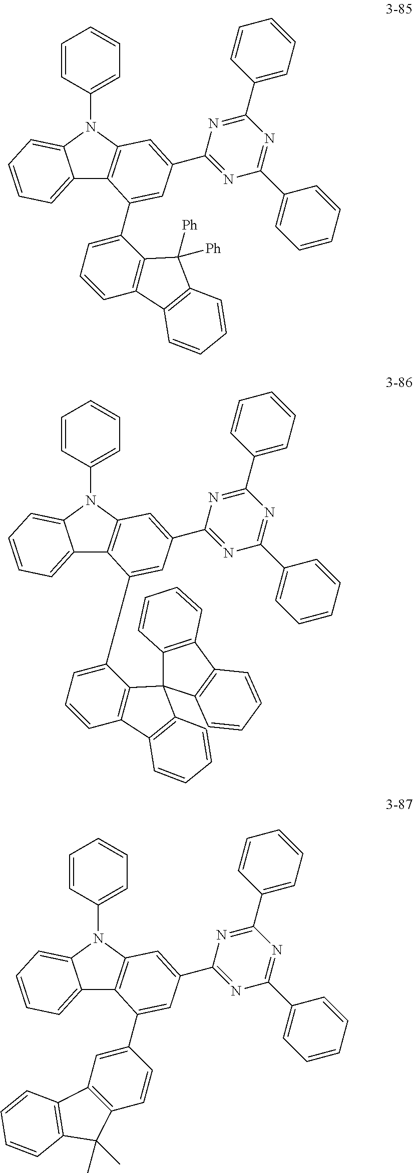 [SMILES: c1ccc(-c2nc(-c3ccccc3)nc(-c3cc(-c4cccc5c4C(c4ccccc4)(c4ccccc4)c4ccccc4-5)c4c5ccccc5n(-c5ccccc5)c4c3)n2)cc1 ; c1ccc(-c2nc(-c3ccccc3)nc(-c3cc(-c4cccc5c4C4(c6ccccc6-c6ccccc64)c4ccccc4-5)c4c5ccccc5n(-c5ccccc5)c4c3)n2)cc1 ; CC1(C)c2ccccc2-c2cc(-c3cc(-c4nc(-c5ccccc5)nc(-c5ccccc5)n4)cc4c3c3ccccc3n4-c3ccccc3)ccc21]

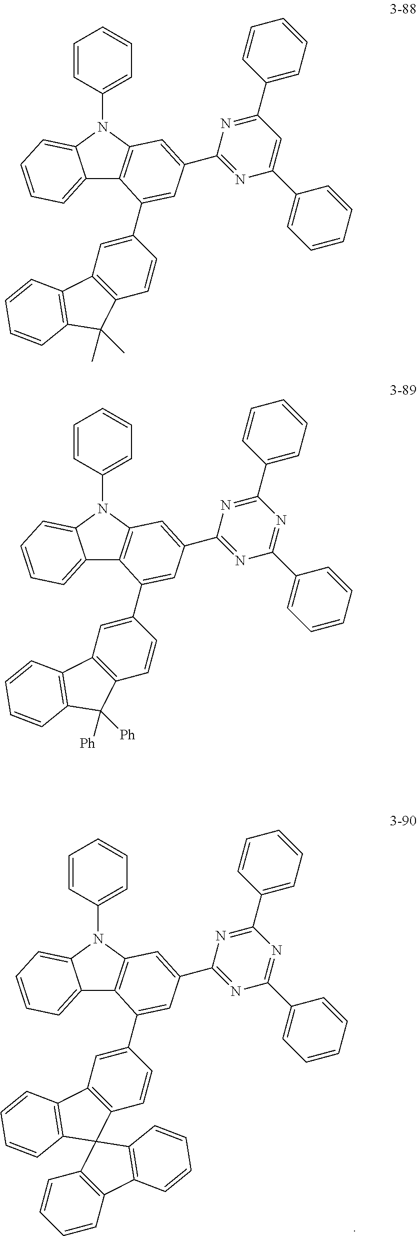 [SMILES: CC1(C)c2ccccc2-c2cc(-c3cc(-c4nc(-c5ccccc5)cc(-c5ccccc5)n4)cc4c3c3ccccc3n4-c3ccccc3)ccc21 ; c1ccc(-c2nc(-c3ccccc3)nc(-c3cc(-c4ccc5c(c4)-c4ccccc4C5(c4ccccc4)c4ccccc4)c4c5ccccc5n(-c5ccccc5)c4c3)n2)cc1 ; c1ccc(-c2nc(-c3ccccc3)nc(-c3cc(-c4ccc5c(c4)-c4ccccc4C54c5ccccc5-c5ccccc54)c4c5ccccc5n(-c5ccccc5)c4c3)n2)cc1]

8. An organic light emitting device comprising:
a first electrode;
a second electrode provided opposite to the first electrode; and
one or more organic material layers provided between the first electrode and the second electrode,
wherein one or more layers of the organic material layers comprise the heterocyclic compound of claim 1.

9. The organic light emitting device of claim 8, wherein the organic material layer comprising the heterocyclic compound further comprises a heterocyclic compound represented by the following Chemical Formula 2: 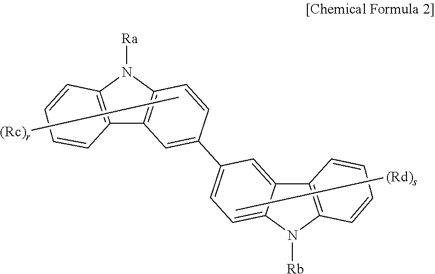
in Chemical Formula 2,
Rc and Rd are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiR₁₀R₁₁R₁₂; —P(═O)R₁₀R₁₁; and an amine group unsubstituted or substituted with a substituted or unsubstituted alkyl group, a substituted or unsubstituted aryl group or a substituted or unsubstituted heteroaryl group, or two or more groups adjacent to each other bond to each other to form a substituted or unsubstituted aromatic hydrocarbon ring or a substituted or unsubstituted heteroring;
R₁₀, R₁₁ and R₁₂ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group;
Ra and Rb are the same as or different from each other, and each independently a substituted or unsubstituted aryl group; or a substituted or unsubstituted heteroaryl group; and
r and s are an integer of 0 to 7.

10. The organic light emitting device of claim 9, wherein the heterocyclic compound represented by Chemical Formula 2 is any one selected from among the following compounds:

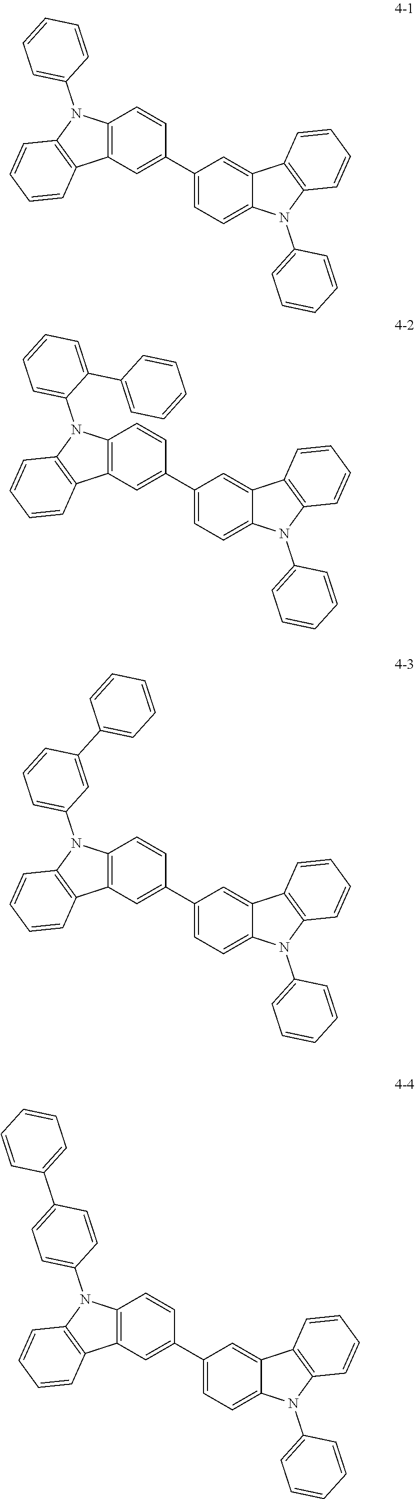 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

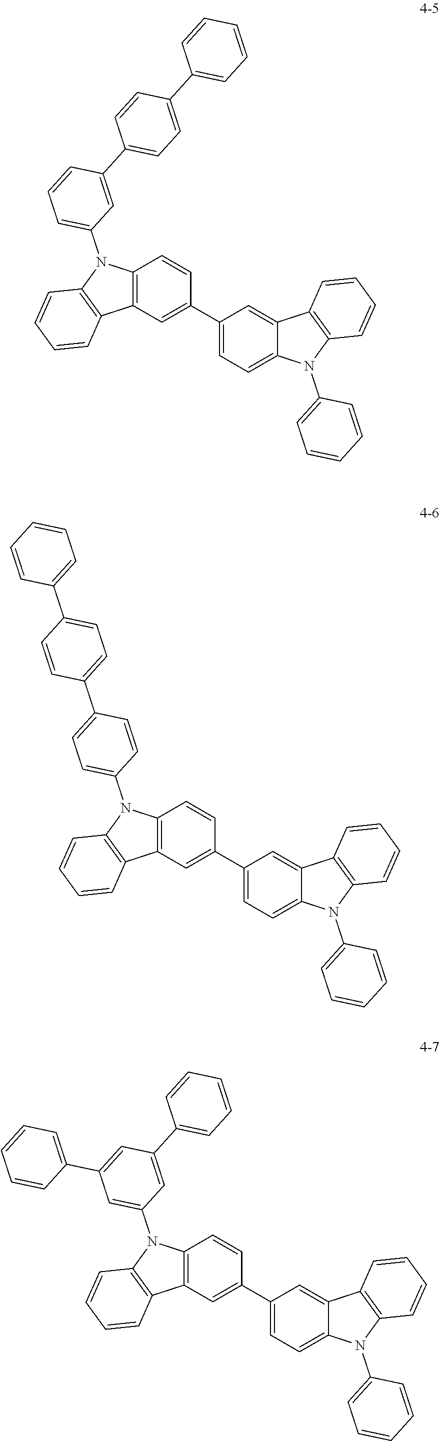 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

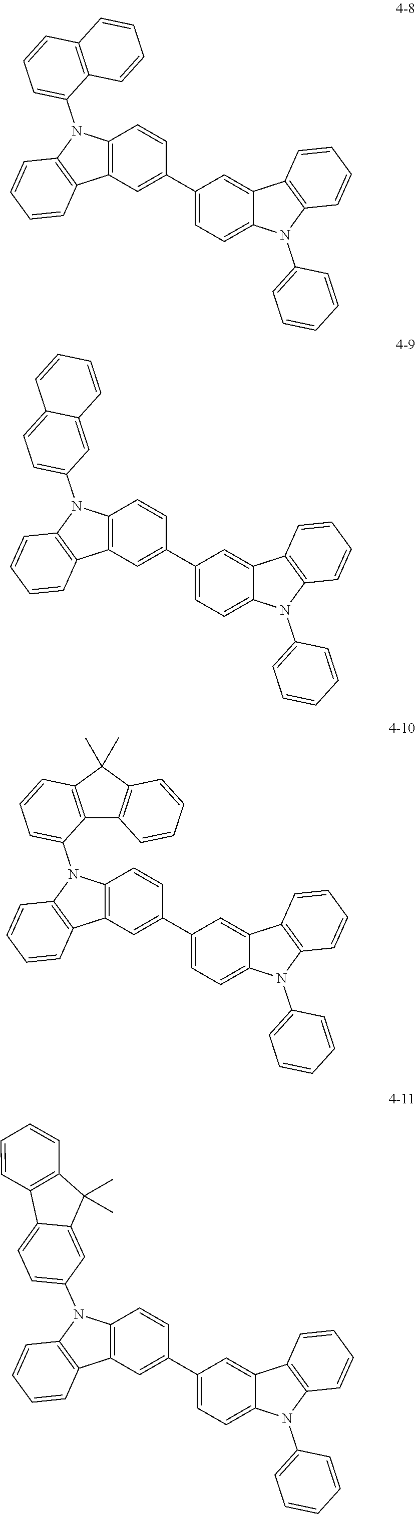 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21]

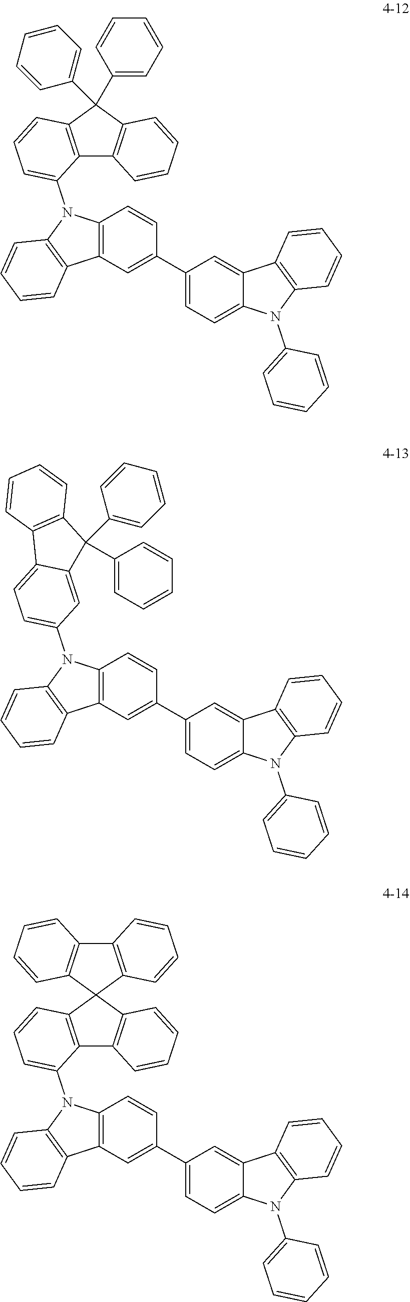 [SMILES: c1ccc(-n2c3ccccc3c3cc(-c4ccc5c(c4)c4ccccc4n5-c4cccc5c4-c4ccccc4C5(c4ccccc4)c4ccccc4)ccc32)cc1 ; c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1]

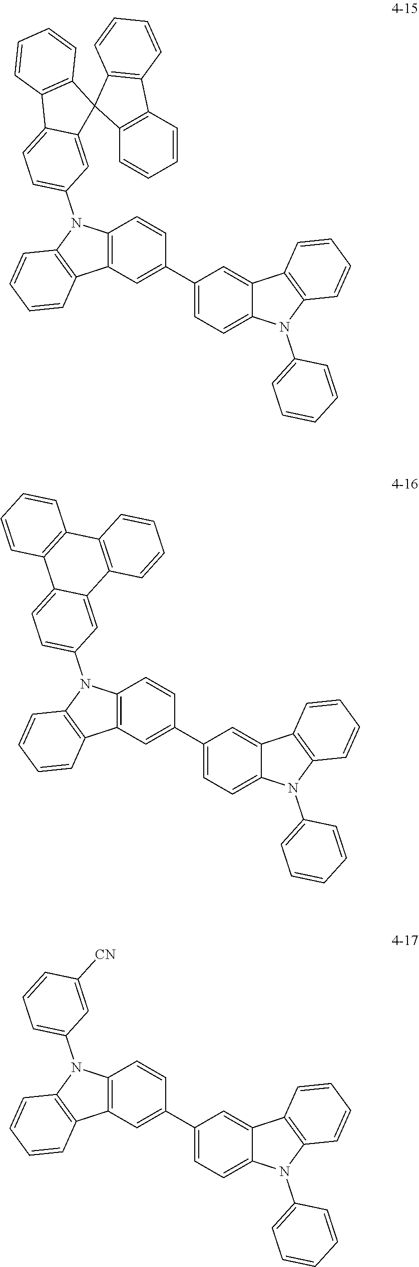 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1]

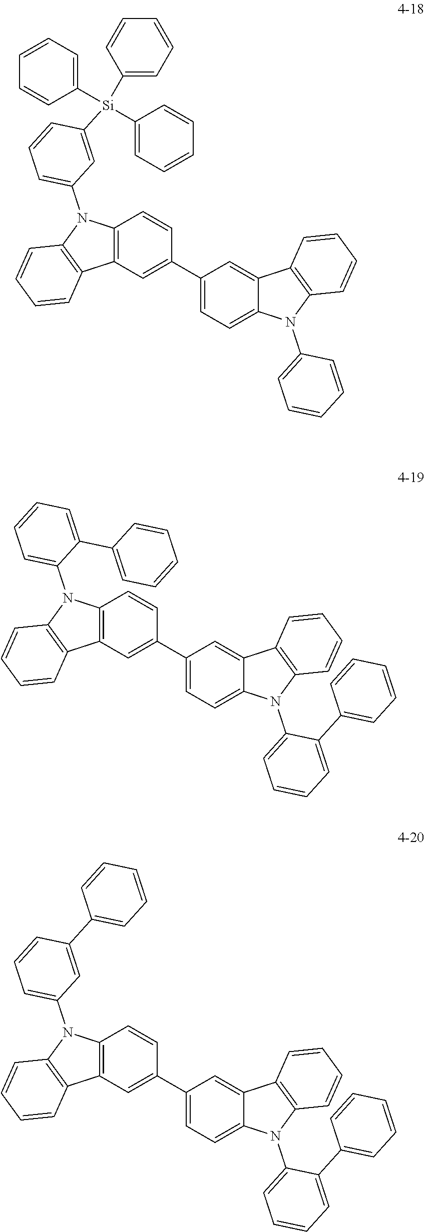 [SMILES: c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1]

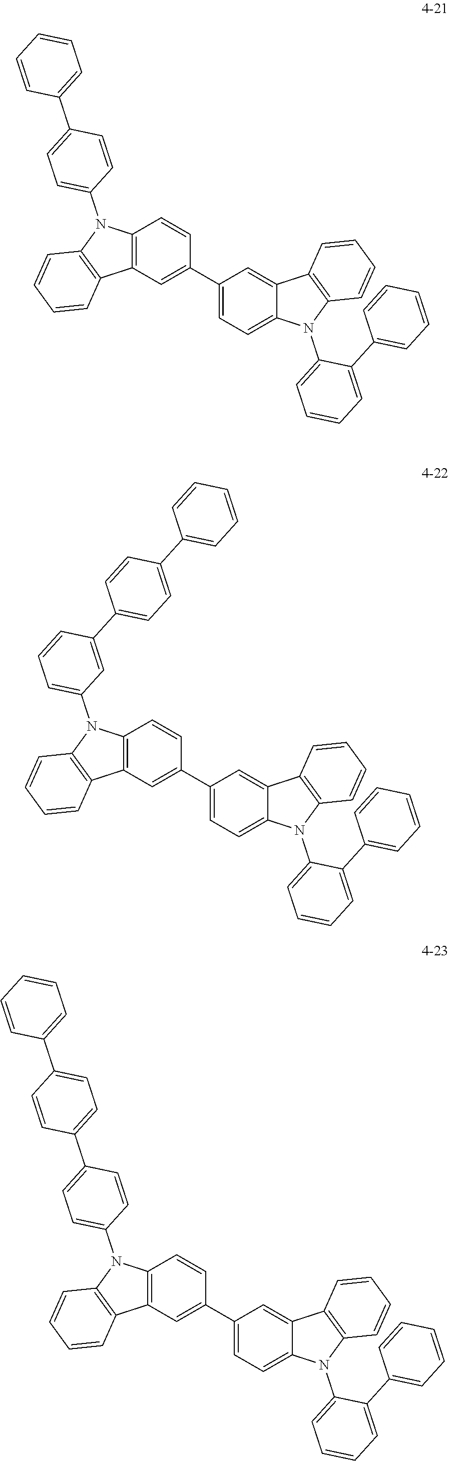 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1]

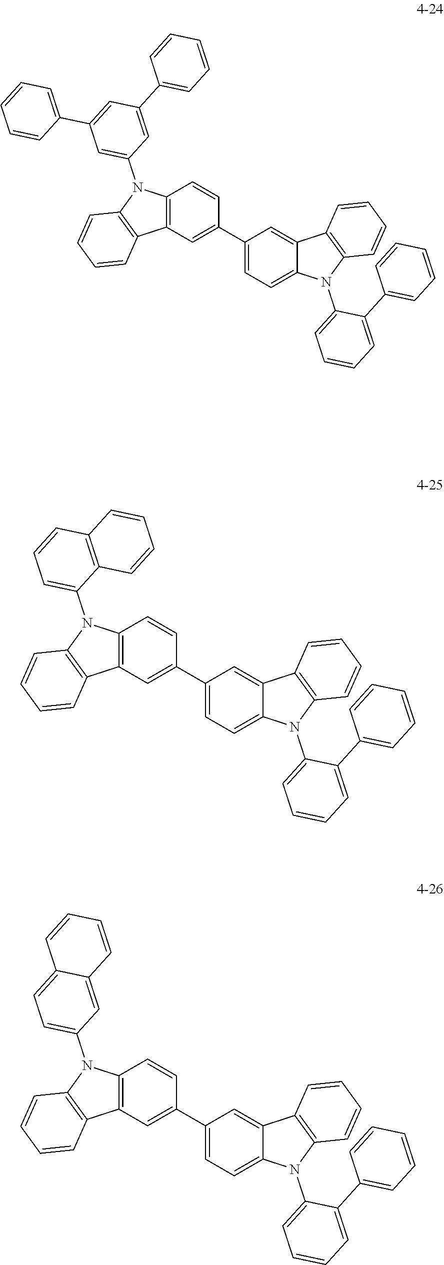 [SMILES: c1ccc(-c2cc(-c3ccccc3)cc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4cccc5ccccc45)ccc32)cc1 ; c1ccc(-c2ccccc2-n2c3ccccc3c3cc(-c4ccc5c(c4)c4ccccc4n5-c4ccc5ccccc5c4)ccc32)cc1]

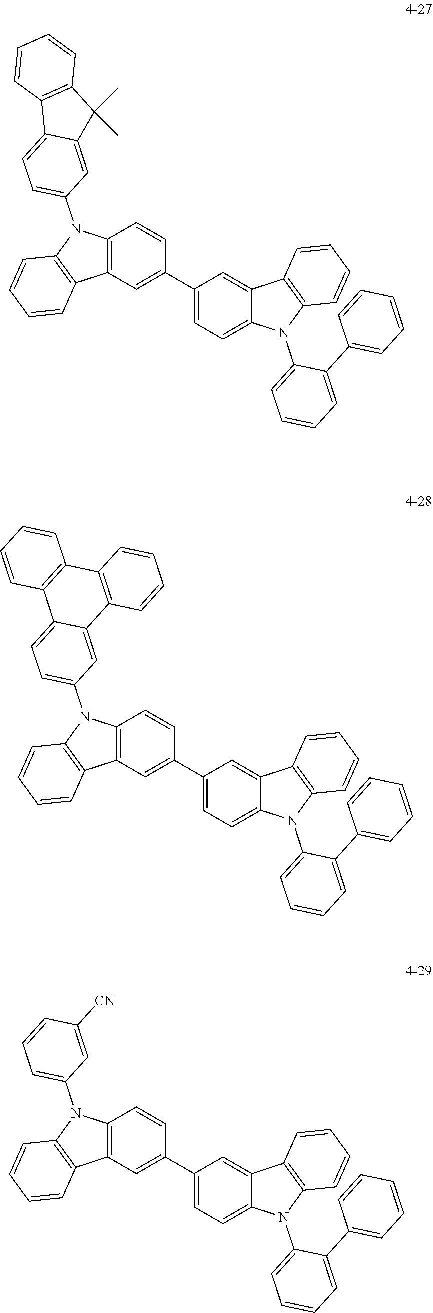

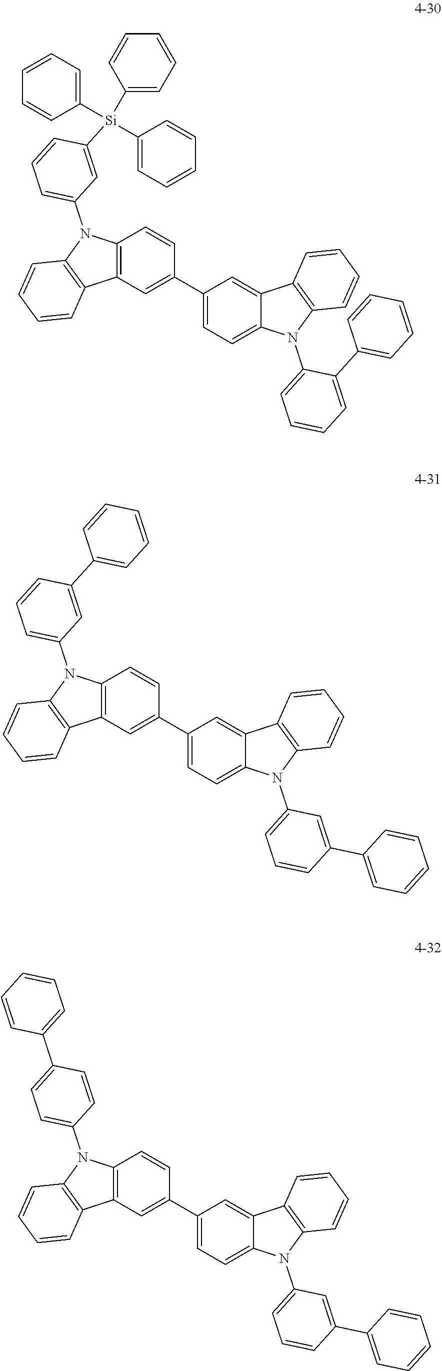

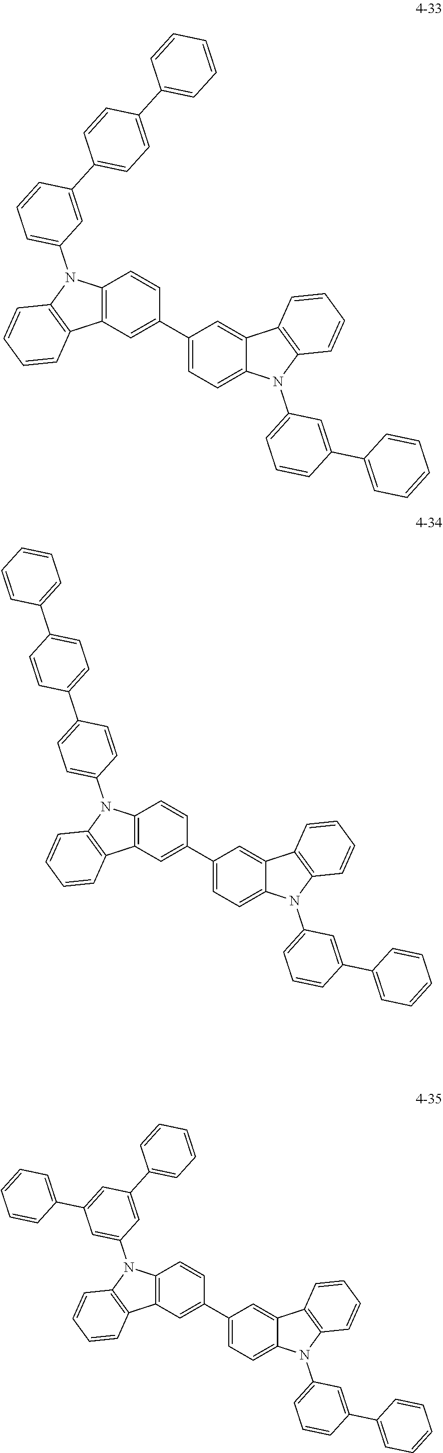

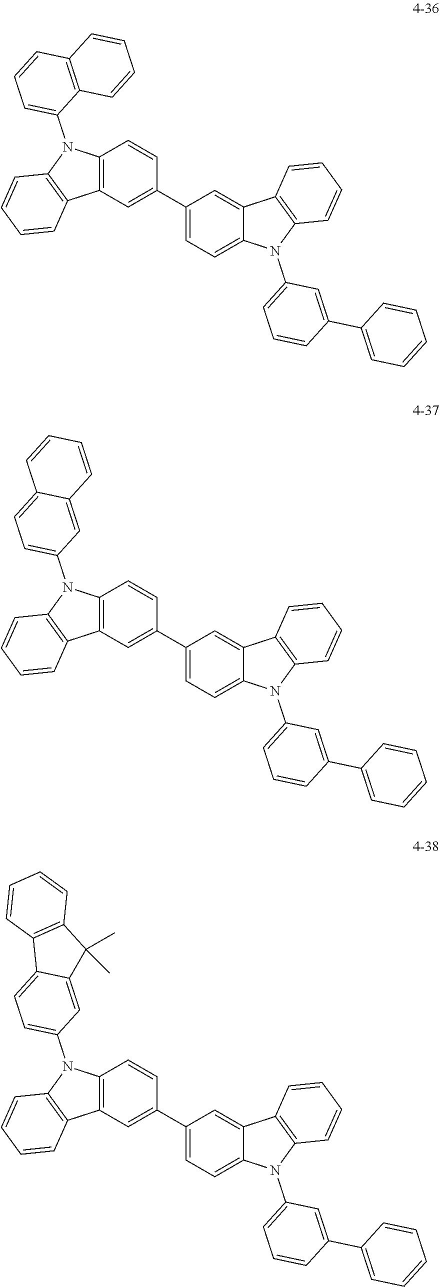

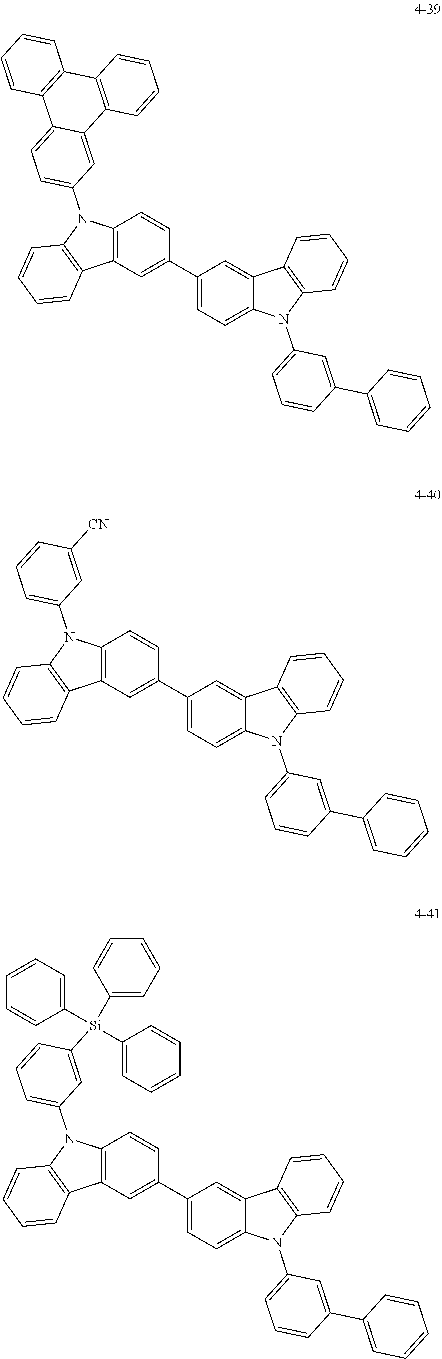

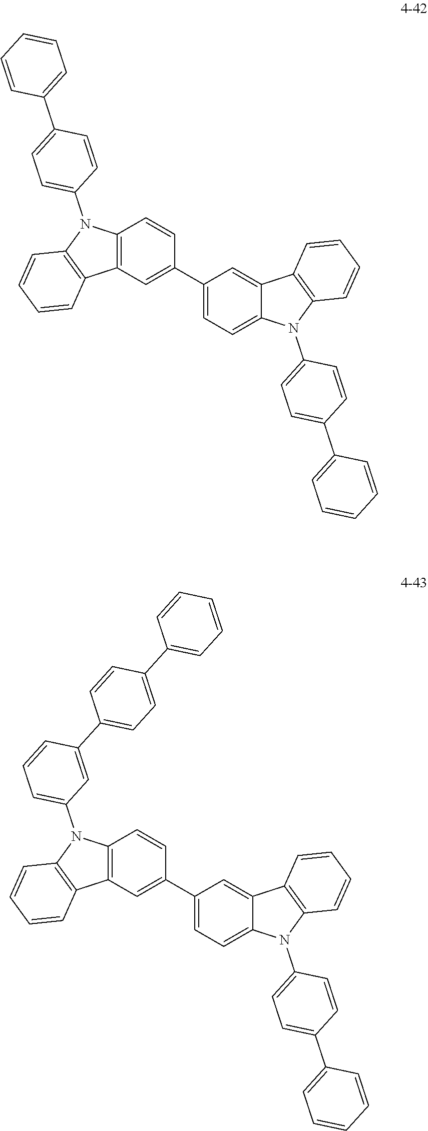

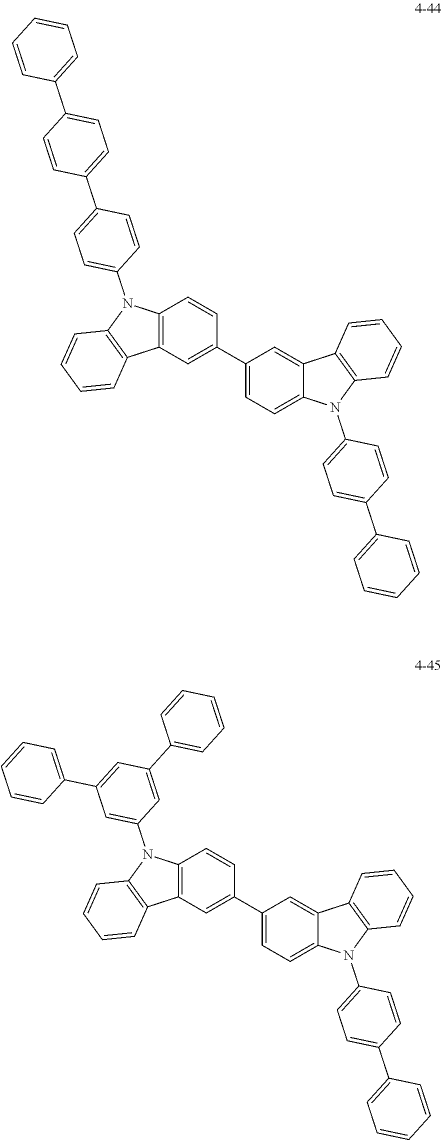

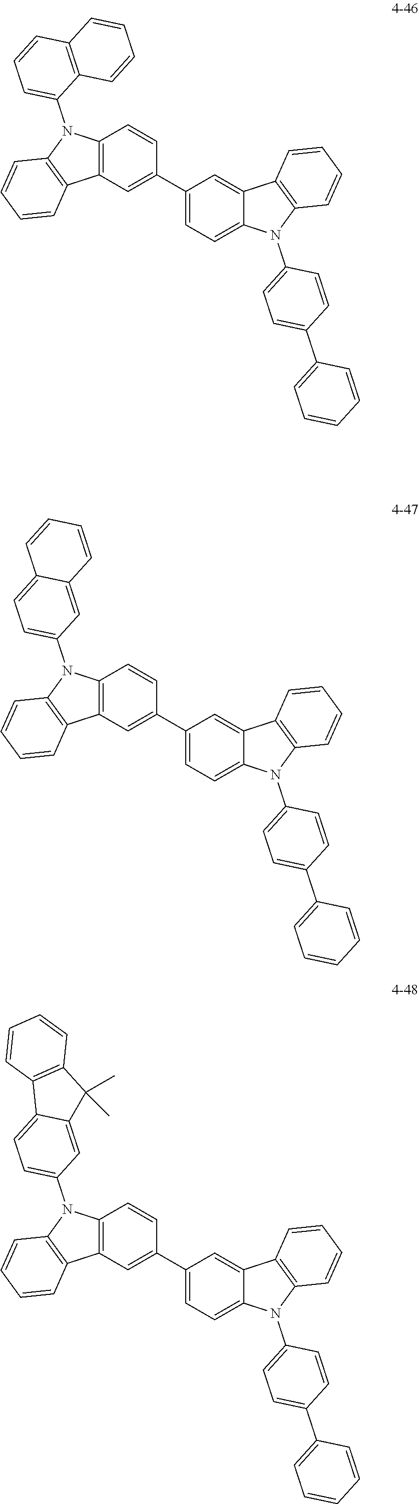

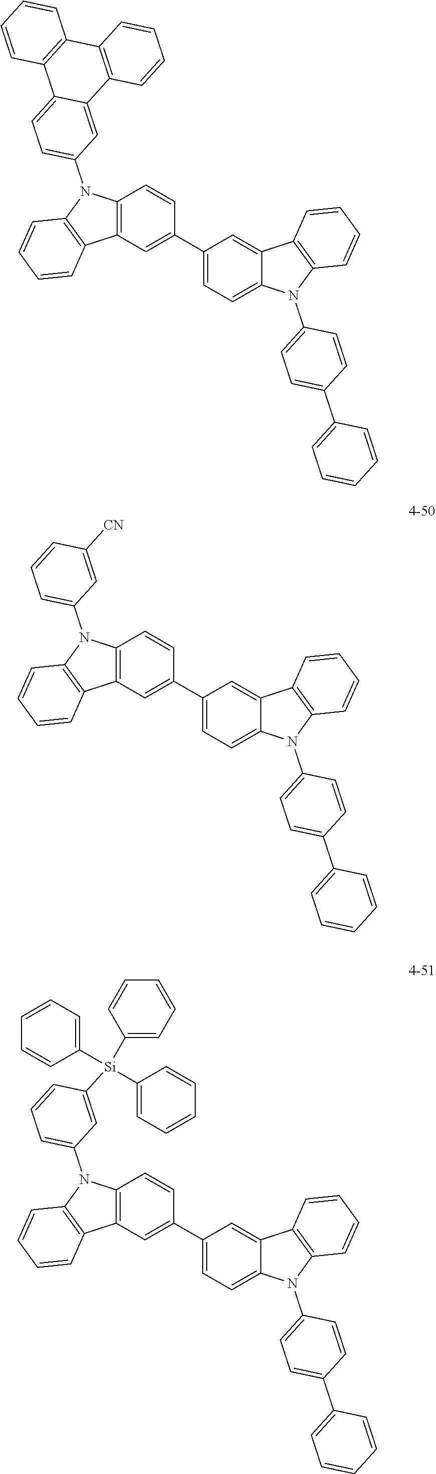

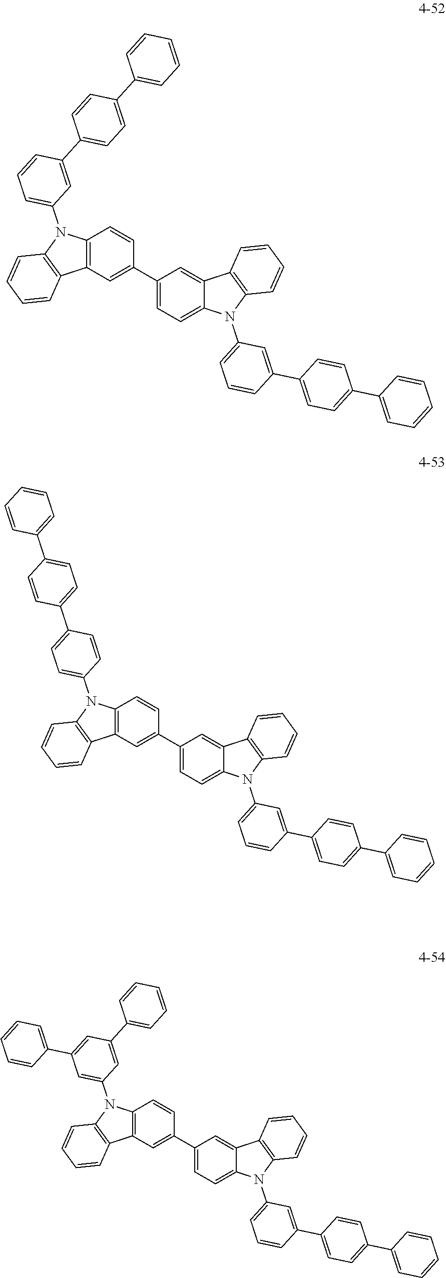

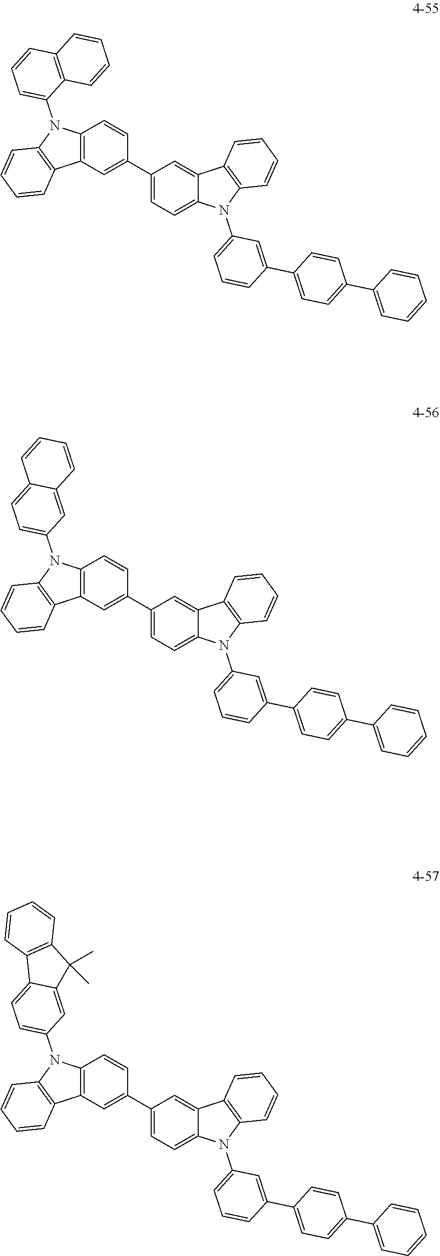 [SMILES: c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1 ; CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21]

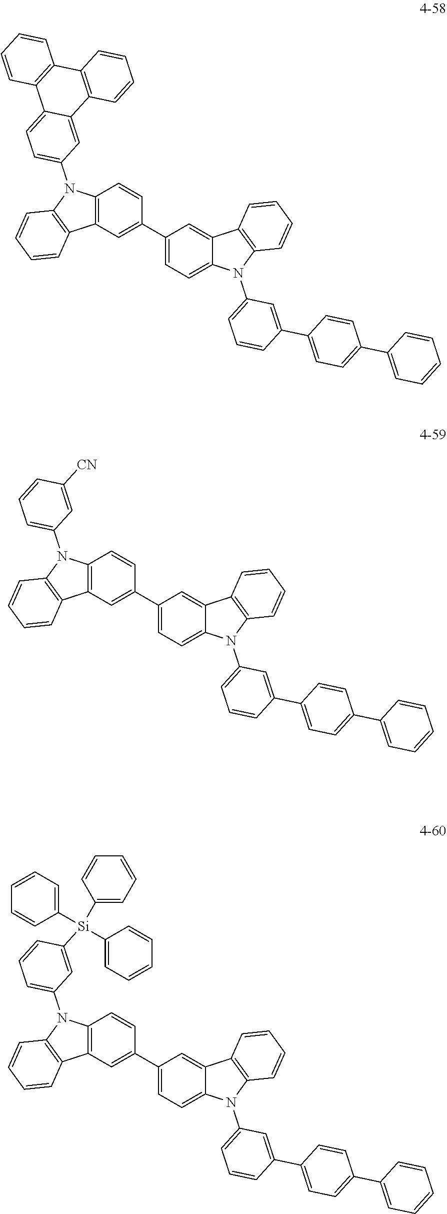 [SMILES: c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1 ; N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1]

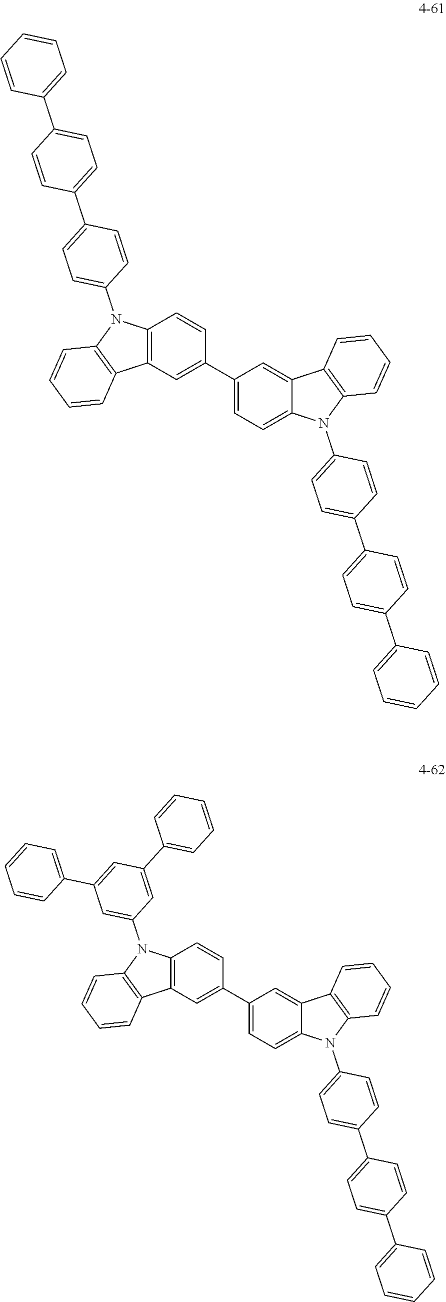 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1]

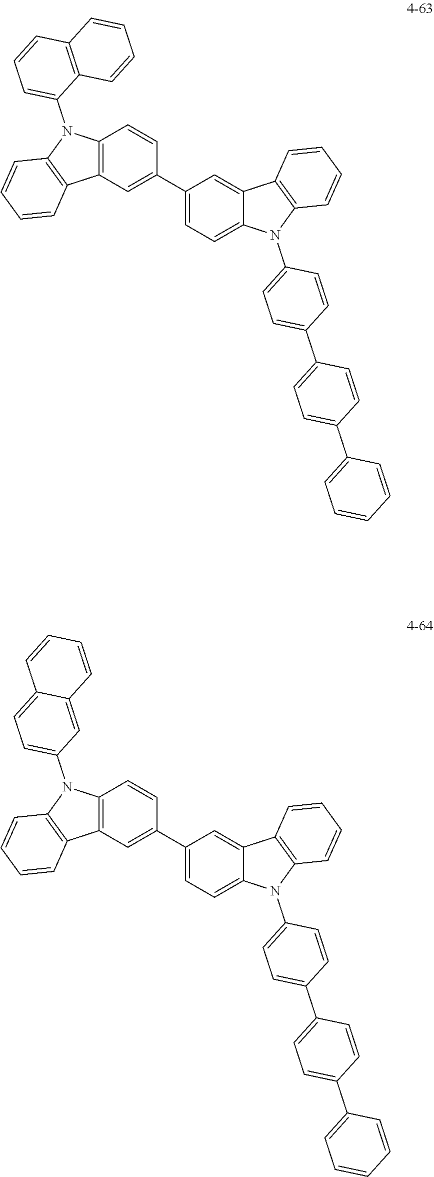 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

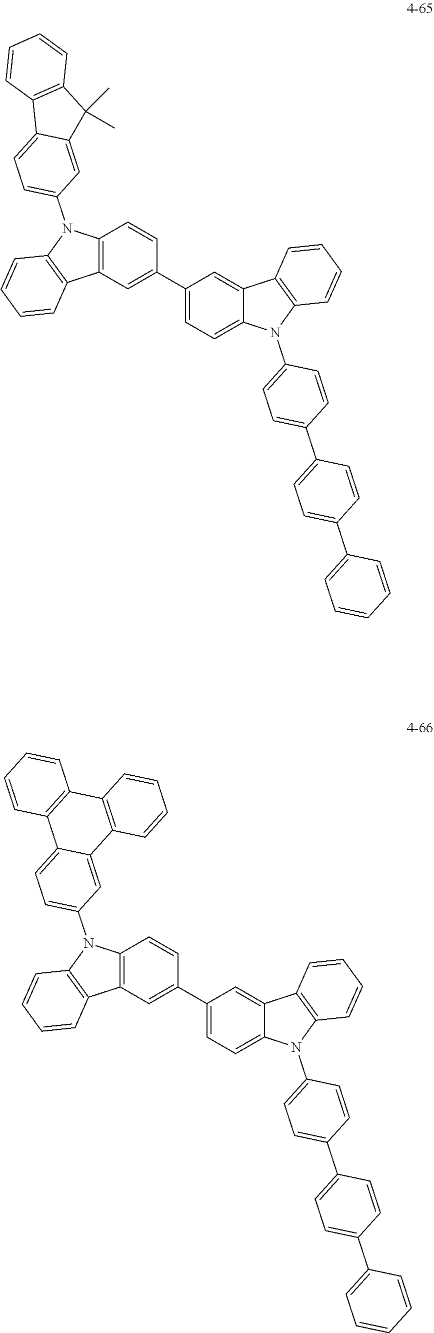 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

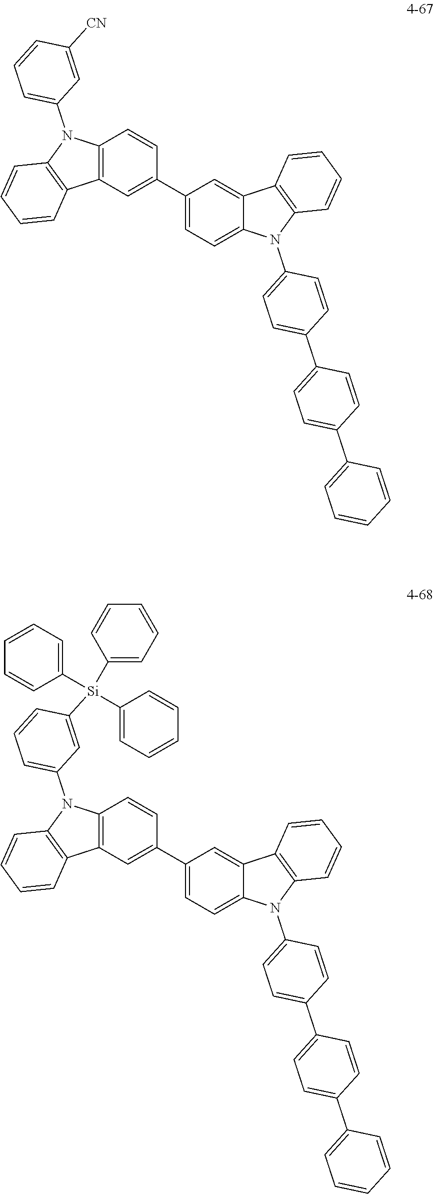 [SMILES: N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

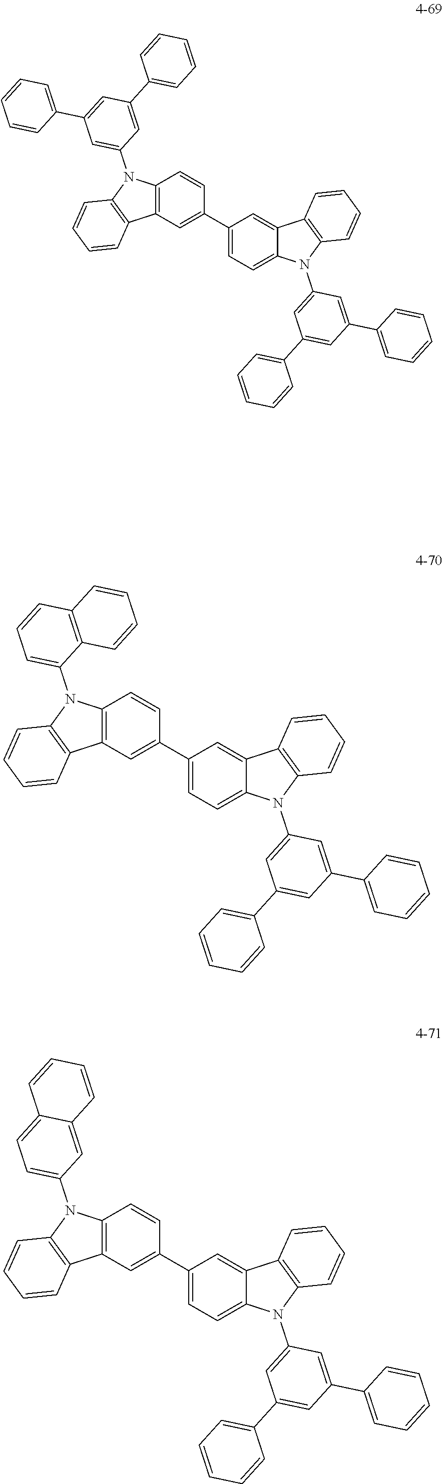

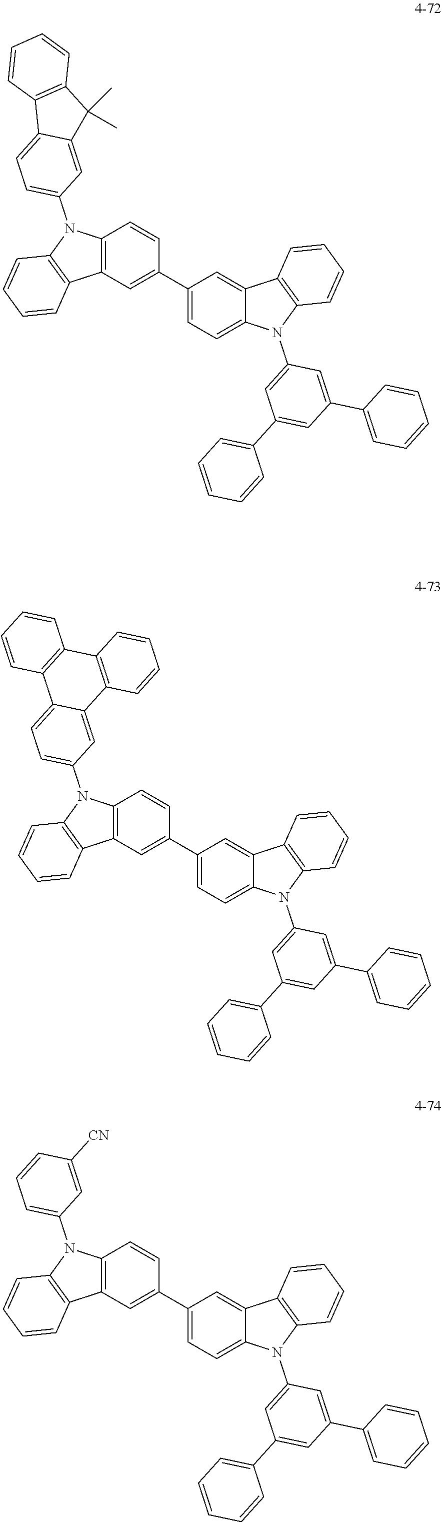

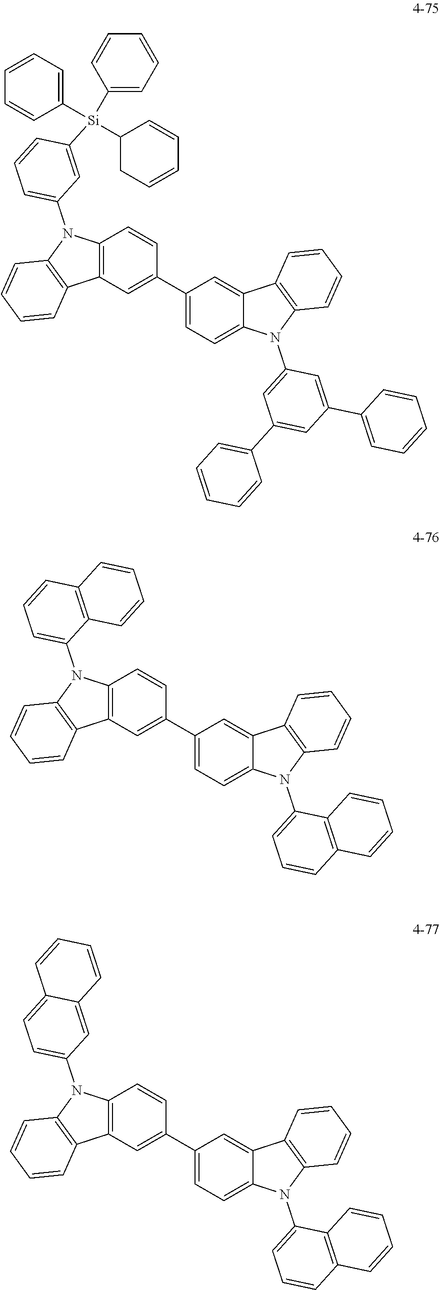 [SMILES: C1=CCC([Si](c2ccccc2)(c2ccccc2)c2cccc(-n3c4ccccc4c4cc(-c5ccc6c(c5)c5ccccc5n6-c5cc(-c6ccccc6)cc(-c6ccccc6)c5)ccc43)c2)C=C1 ; c1ccc2c(-n3c4ccccc4c4cc(-c5ccc6c(c5)c5ccccc5n6-c5cccc6ccccc56)ccc43)cccc2c1 ; c1ccc2cc(-n3c4ccccc4c4cc(-c5ccc6c(c5)c5ccccc5n6-c5cccc6ccccc56)ccc43)ccc2c1]

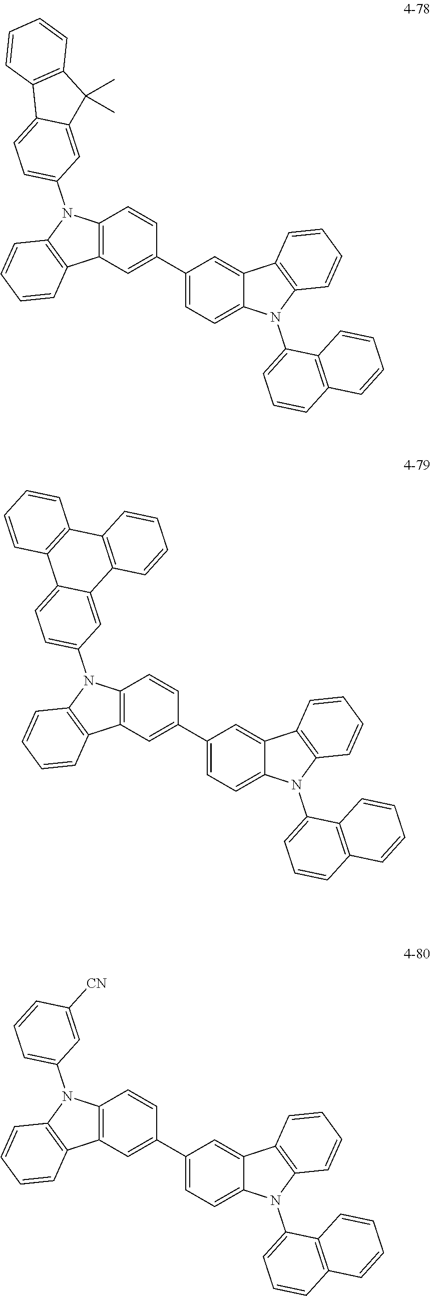

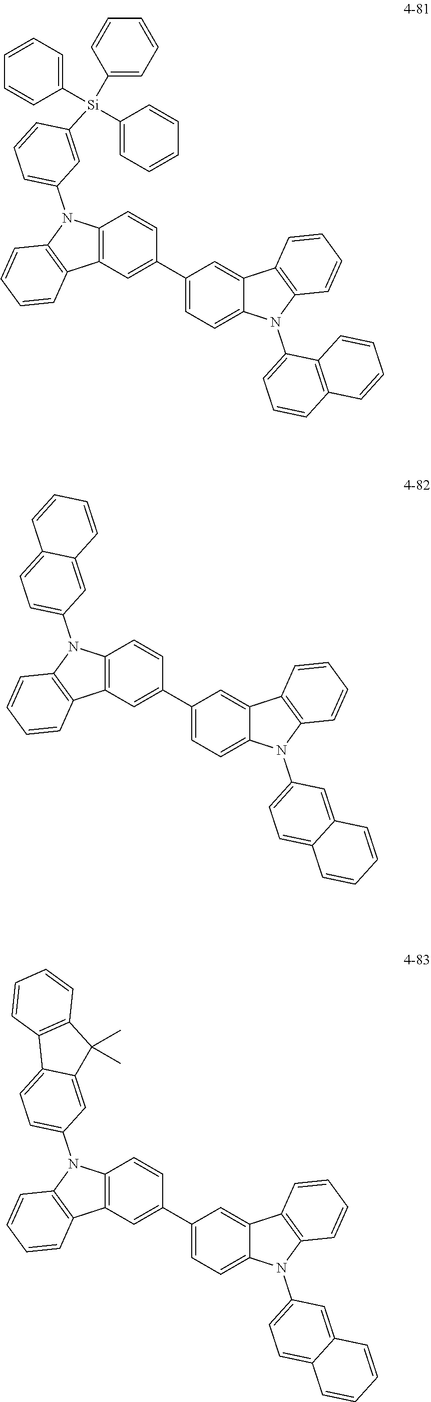

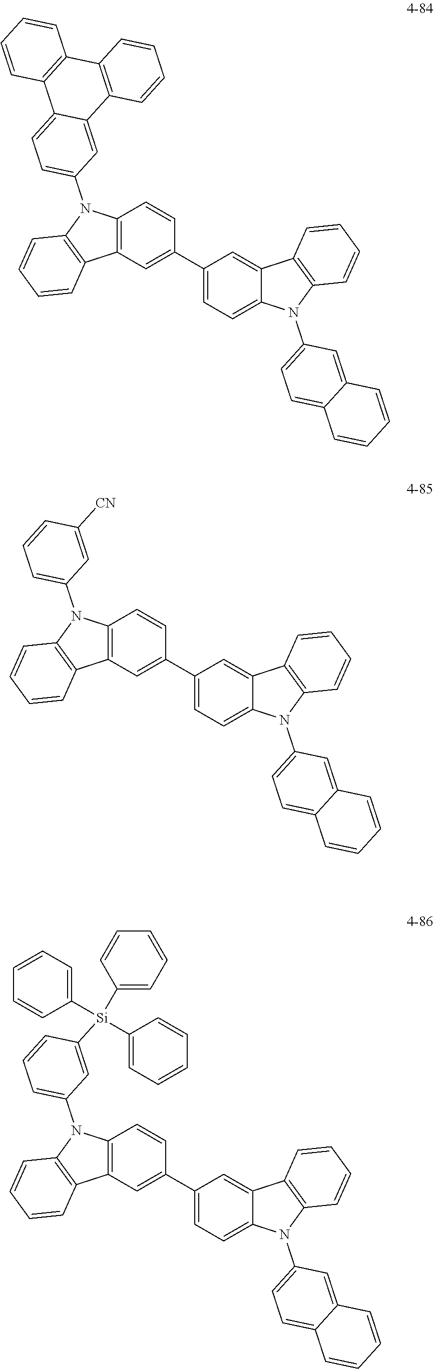

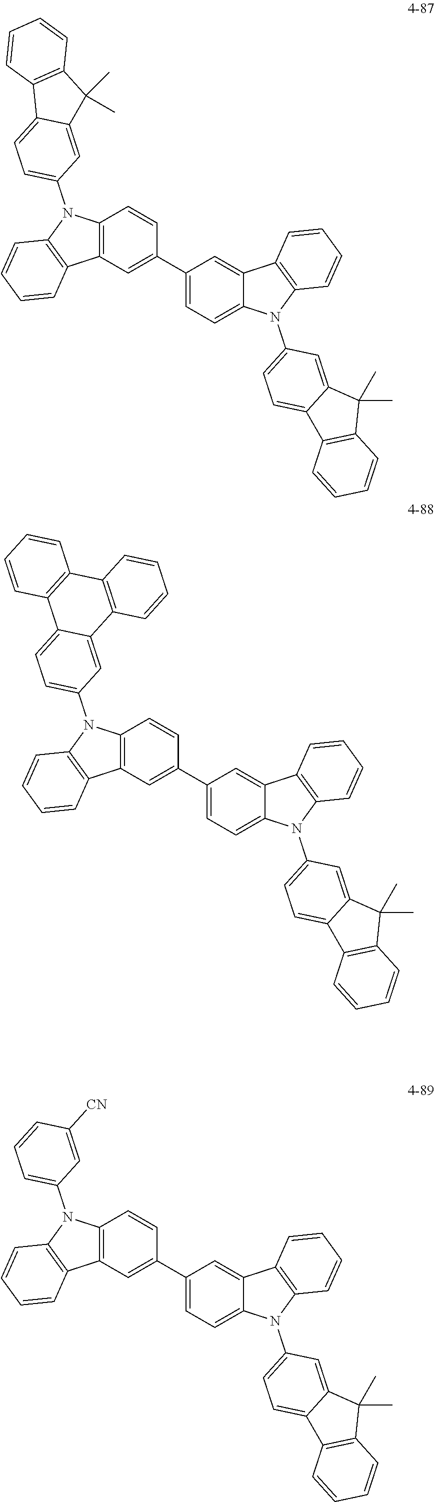 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21]

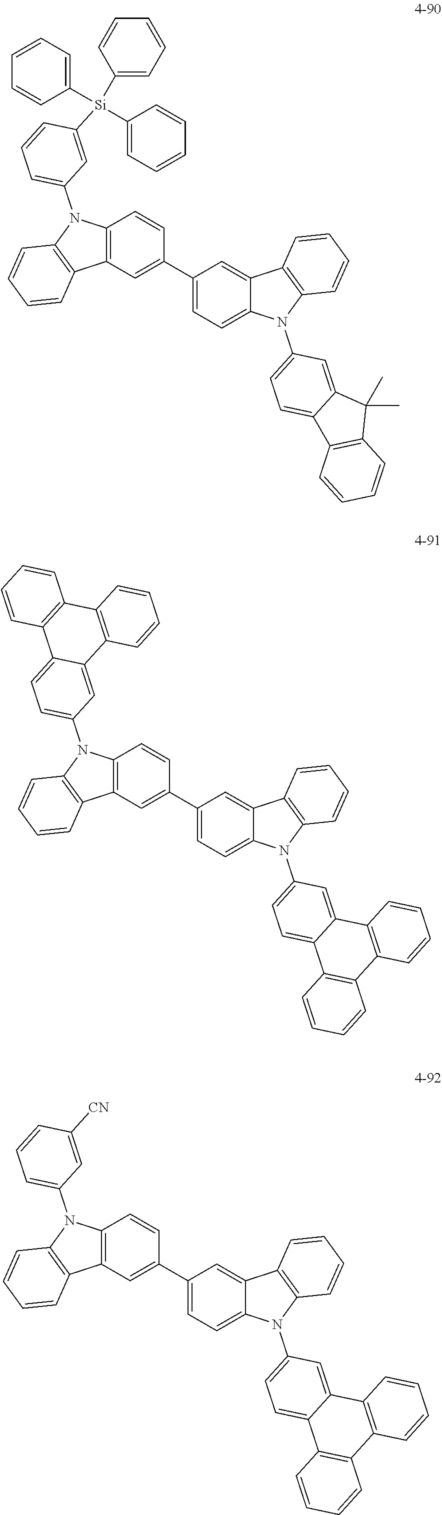 [SMILES: CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21 ; c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1]

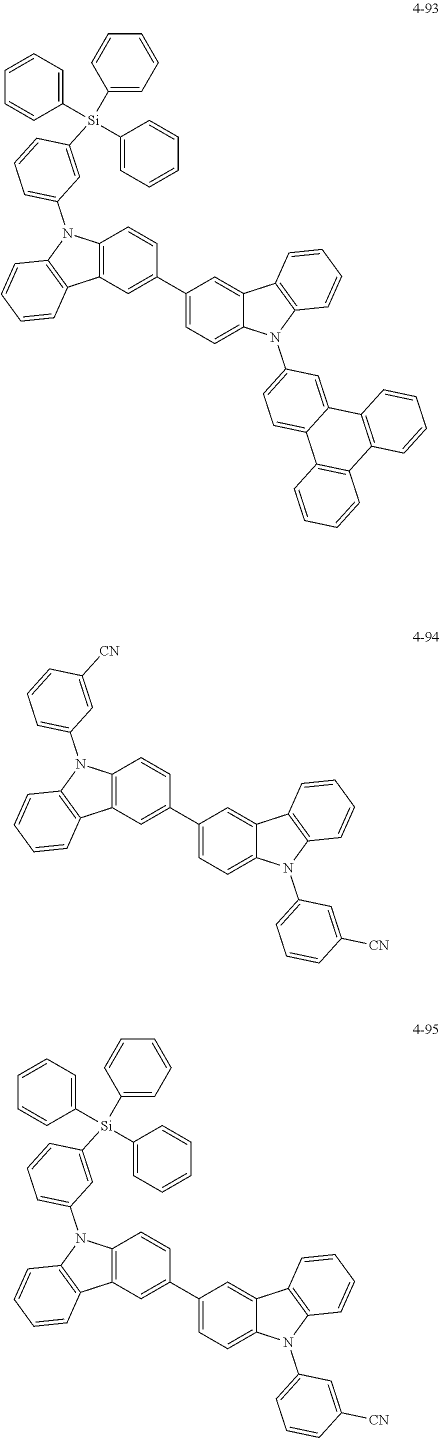 [SMILES: c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1 ; N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1]

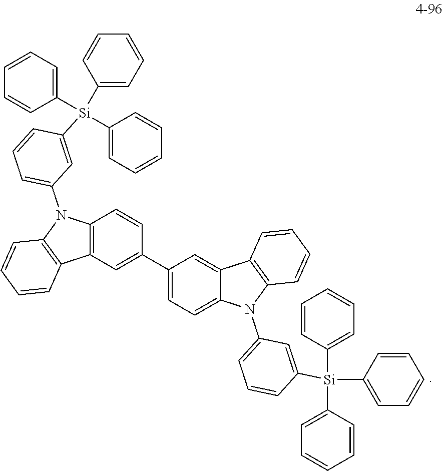 [SMILES: c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

11. The organic light emitting device of claim 9, wherein Rc and Rd are hydrogen.

12. The organic light emitting device of claim 9, wherein Ra and Rb are the same as or different from each other, and each independently a substituted or unsubstituted C6 to C40 aryl group.

13. The organic light emitting device of claim 8, wherein the organic material layer comprises a light emitting layer, and the light emitting layer comprises the heterocyclic compound.

14. The organic light emitting device of claim 8, wherein the organic material layer comprises a light emitting layer, the light emitting layer comprises a host material, and the host material comprises the heterocyclic compound.

15. The organic light emitting device of claim 8, further comprising one, two or more layers selected from the group consisting of a light emitting layer, a hole injection layer, a hole transfer layer, an electron injection layer, an electron transfer layer, an electron blocking layer and a hole blocking layer.

16. A composition for an organic material layer of an organic light emitting device, the composition comprising:
the heterocyclic compound of claim 1; and
a heterocyclic compound represented by the following Chemical Formula 2: 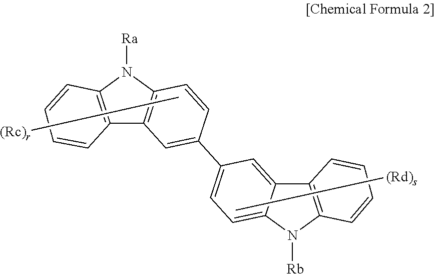
wherein, in Chemical Formula 2,
Rc and Rd are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiR₁₀R₁₁R₁₂; —P(═O)R₁₀R₁₁; and an amine group unsubstituted or substituted with a substituted or unsubstituted alkyl group, a substituted or unsubstituted aryl group or a substituted or unsubstituted heteroaryl group, or two or more groups adjacent to each other bond to each other to form a substituted or unsubstituted aromatic hydrocarbon ring or a substituted or unsubstituted heteroring;
R₁₀, R₁₁ and R₁₂ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group;
Ra and Rb are the same as or different from each other, and each independently a substituted or unsubstituted aryl group; or a substituted or unsubstituted heteroaryl group; and
r and s are an integer of 0 to 7.

17. The composition for an organic material layer of an organic light emitting device of claim 16, wherein, in the composition, the heterocyclic compound:the heterocyclic compound represented by Chemical Formula 2 have a weight ratio of 1:10 to 10:1.

18. A method for manufacturing an organic light emitting device, the method comprising:
preparing a substrate;
forming a first electrode on the substrate;
forming one or more organic material layers on the first electrode; and
forming a second electrode on the organic material layer,
wherein the forming of organic material layers comprises forming one or more organic material layers using the composition for an organic material layer of claim 16.

19. The method for manufacturing an organic light emitting device of claim 18, wherein the forming of organic material layers is forming using a thermal vacuum deposition method after pre-mixing the heterocyclic compound of Chemical Formula 1 and the heterocyclic compound of Chemical Formula 2.
